(19) United States
(12) Patent Application Publication
(10) Pub. No.: US 2023/0011652 A1
(43) Pub. Date: Jan. 12, 2023

(54) COMPOUNDS AND COMPOSITIONS FOR TREATING CONDITIONS ASSOCIATED WITH NLRP ACTIVITY

(71) Applicant: Novartis AG, Basel (CH)

(72) Inventors: William ROUSH, Boston, MA (US); Shankar VENKATRAMAN, Lansdale, PA (US); Shomir GHOSH, Brookline, MA (US); Dong-Ming SHEN, Edison, NJ (US); Jason KATZ, Newton, MA (US); Hans Martin SEIDEL, Concord, MA (US)

(21) Appl. No.: 17/287,834

(22) PCT Filed: Oct. 23, 2019
(86) PCT No.: PCT/US2019/057682

§ 371 Date: Apr. 22, 2021

Related U.S. Application Data
(60) Provisional application No. 62/749,712, filed on Oct. 24, 2018.
(60) Provisional application No. 62/749,725, filed on Oct. 24, 2018.
(60) Provisional application No. 62/749,730, filed on Oct. 24, 2018.
(60) Provisional application No. 62/751,301, filed on Oct. 26, 2018.
(60) Provisional application No. 62/751,380, filed on Oct. 26, 2018.
(60) Provisional application No. 62/751,386, filed on Oct. 26, 2018.
(60) Provisional application No. 62/801,930, filed on Feb. 6, 2019.
(60) Provisional application No. 62/802,637, filed on Feb. 7, 2019.
(60) Provisional application No. 62/802,654, filed on Feb. 7, 2019.
(60) Provisional application No. 62/812,517, filed on Mar. 1, 2019.
(60) Provisional application No. 62/812,794, filed on Mar. 1, 2019.
(60) Provisional application No. 62/812,813, filed on Mar. 1, 2019.

(51) Int. Cl. C07D 498/20; C07D 333/50; C07D 495/04; C07D 498/04; C07D 487/04; C07D 471/04; C07D 513/04

(52) U.S. Cl. CPC C07D 498/20; C07D 333/50; C07D 495/04; C07D 513/04; C07D 487/04; C07D 471/04; C07D 498/04

(57) ABSTRACT

In one aspect, compounds of Formula AA, or a pharmaceutically acceptable salt thereof, are featured or a pharmaceutically acceptable salt thereof, wherein the variables shown in Formula A can be as defined anywhere herein.

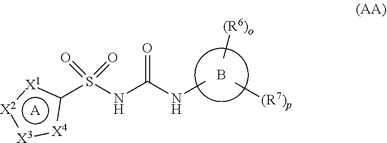

### TECHNICAL FIELD

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) contributes to the pathology and/or symptoms and/or progression of the condition, disease or disorder in a subject (e.g., a human). This disclosure also features compositions as well as other methods of using and making the same.

The present disclosure also relates to, in part, methods and compositions for treating anti-TNFα resistance in a subject with an NLRP3 antagonist. The present disclosure also relates, in part, to methods, combinations and compositions for treating TFNα related diseases and anti-TNFα resistance in a subject that include administration of an NLRP3 antagonist, an NLRP3 antagonist and an anti-TNFα agent, or a composition encompassing an NLRP3 antagonist and an anti-TNFα agent.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

NLRP3 can form a complex and has been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NLRP3.

Several patients having inflammatory or autoimmune diseases are treated with anti-TNFα agents. A subpopulation of such patients develop resistance to treatment with the anti-TNFα agents. It is desirable to develop methods for reducing a patient's resistance to anti-TNFα agents. In light of the this, it would also be desirable to provide alternative therapies for treating inflammatory or autoimmune diseases (for example NLRP3 inflammasome inhibitors) to avoid or minimise the use of anti-TNFα agents.

Intestinal bowel disease (IBD), encompassing Ulcerative Colitis (UC) and Crohn's disease (CD), are chronic diseases characterized by barrier dysfunction and uncontrolled inflammation and mucosal immune reactions in the gut. A number of inflammatory pathways have been implicated in the progression of IBD, and anti-inflammatory therapy such as tumor necrosis factor-alpha (TNF-α) blockade has shown efficacy in the clinic (Rutgeerts P et al N Engl J Med 2005; 353:2462-76). Anti-TNFα therapies, however, do not show complete efficacy, however, other cytokines such as IL-1 b, IL-6, IL-12, IL-18, IL-21, and IL-23 have been shown to drive inflammatory disease pathology in IBD (Neurath M F Nat Rev Immunol 2014; 14; 329-42). IL-1b and IL-18 are produced by the NLRP3 inflammasome in response to pathogenic danger signals, and have been shown to play a role in IBD. Anti-IL-1b therapy is efficacious in patients with IBD driven by genetic mutations in CARD8 or IL-10R (Mao L et al, J Clin Invest 2018; 238:1793-1806, Shouval D S et al, Gastroenterology 2016; 151:1100-1104), IL-18 genetic polymorphisms have been linked to UC (Kanai T et al, Curr Drug Targets 2013; 14:1392-9), and NLRP3 inflammasome inhibitors have been shown to be efficacious in murine models of IBD (Perera A P et al, Sci Rep 2018; 8:8618). Resident gut immune cells isolated from the lamina propria of IBD patients can produce IL-1b, either spontaneously or when stimulated by LPS, and this IL-1b production can be blocked by the ex vivo addition of a NLRP3 antagonist. Based on strong clinical and preclinical evidence showing that inflammasome-driven IL-1b and IL-18 play a role in IBD pathology, it is clear that NLRP3 inflammasome inhibitors could be an efficacious treatment option for UC, Crohn's disease, or subsets of IBD patients. These subsets of patients could be defined by their peripheral or gut levels of inflammasome related cytokines including IL-1b, IL-6, and IL-18, by genetic factors that pre-dispose IBD patients to having NLRP3 inflammasome activation such as mutations in genes including ATG16L1, CARD8, IL-10R, or PTPN2 (Saitoh T et al, Nature 2008; 456:264, Spalinger M R, Cell Rep 2018; 22:1835), or by other clinical rationale such as non-response to TNF therapy.

Though anti-TNF therapy is an effective treatment option for Crohn's disease, 40% of patients fail to respond. One-third of non-responsive CD patients fail to respond to anti-TNF therapy at the onset of treatment, while another third lose response to treatment over time (secondary non-response). Secondary non-response can be due to the generation of anti-drug antibodies, or a change in the immune compartment that desensitizes the patient to anti-TNF (Ben-Horin S et al, Autoimmun Rev 2014; 13:24-30, Steenholdt C et al Gut 2014; 63:919-27). Anti-TNF reduces inflammation in IBD by causing pathogenic T cell apoptosis in the intestine, therefore eliminating the T cell mediated inflammatory response (Van den Brande et al Gut 2007:56:509-17). There is increased NLRP3 expression and increased production of IL-1b in the gut of TNF-non-responsive CD patients (Leal R F et al Gut 2015; 64:233-42) compared to TNF-responsive patients, suggesting NLRP3 inflammasome pathway activation. Furthermore, there is increased expression of TNF-receptor 2 (TNF-R2), which allows for TNF-mediated proliferation of T cells (Schmitt H et al Gut 2018; 0:1-15). IL-1b signaling in the gut promotes T cell differentiation toward Th1/17 cells which can escape anti-TNF-α mediated apoptosis. It is therefore likely that NLRP3 inflammasome activation can cause non-responsiveness in CD patients to anti-TNF-α therapy by sensitizing pathogenic T cells in the gut to anti-TNF-α mediated apoptosis. Experimental data from immune cells isolated from the gut of TNF-resistant Crohn's patients show that these cells spontaneously release IL-1b, which can be inhibited by the addition of an NLRP3 antagonist. NLRP3 inflammasome antagonists—in part by blocking IL-1b secretion—would be expected to inhibit the mechanism leading to anti-TNF non-responsiveness, re-sensitizing the patient to anti-TNF therapy. In IBD patients who are naive to anti-TNF therapy, treatment with an NLRP3 antagonist would be expected to prevent primary- and secondary-non responsiveness by blocking the mechanism leading to non-response.

NLRP3 antagonists that are efficacious locally in the gut can be efficacious drugs to treat IBD; in particular in the treatment of TNF-resistant CD alone or in combination with anti-TNF therapy. Systemic inhibition of both IL-1b and TNF-α has been shown to increase the risk of opportunistic infections (Genovese M C et al, Arthritis Rheum 2004; 50:1412), therefore, only blocking the NLRP3 inflammasome at the site of inflammation would reduce the infection risk inherent in neutralizing both IL-1□ and TNF-α. NLRP3 antagonists that are potent in NLRP3-inflammasome driven cytokine secretion assays in cells, but have low permeability in vitro in a permeability assay such as an MDCK assay, have poor systemic bioavailability in a rat or mouse pharmacokinetic experiment, but high levels of compound in the colon and/or small intestine could be a useful therapeutic option for gut restricted purposes.

The present invention also provides alternative therapies for the treatment of inflammatory or autoimmune diseases, including IBD, that solves the above problems associated with anti-TNFα agents.

### SUMMARY

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling).

In some embodiments, provided herein is a compound of Formula AA

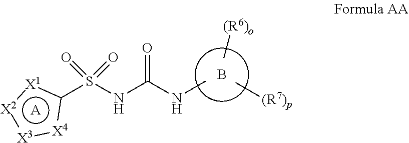

or a pharmaceutically acceptable salt thereof, wherein the variables in Formula AA can be as defined anywhere herein.

This disclosure also features compositions as well as other methods of using and making the same.

The present invention is also relates to the Applicant's discovery that inhibition of NLRP3 inflammasomes can increase a subject's sensitivity to an anti-TNFα agent or can overcome resistance to an anti-TNFα agent in a subject, or indeed provide an alternative therapy to anti-TNFα agents.

Provided herein are methods of treating a subject that include: (a) identifying a subject having a cell that has an elevated level of NLRP3 inflammasome activity and/or expression as compared to a reference level; and (b) administering to the identified subject a therapeutically effective amount of an compound of Formula I or a pharmaceutically acceptable salt, solvate, or co-crystal thereof.

Provided herein are methods for the treatment of inflammatory or autoimmune disease including IBD, such as UC and CD in a subject in need thereof, comprising administering to said subject a therapeutically effective amount a compound for Formula I or a pharmaceutically acceptable salt, solvate, or co-crystal thereof, wherein the NLRP3 antagonist is a gut-targeted NLRP3 antagonist.

Provided herein are methods of treating a subject in need thereof, that include: (a) identifying a subject having resistance to an anti-TNFα agent; and (b) administering a treatment comprising a therapeutically effective amount of a compound for Formula I, or a pharmaceutically acceptable salt, solvate, or co-crystal thereof to the identified subject.

Provided herein are methods of treating a subject in need thereof, that include: administering a treatment comprising a therapeutically effective amount of a compound for Formula I or a pharmaceutically acceptable salt, solvate, or co-crystal thereof to a subject identified as having resistance to an anti-TNFα agent.

Provided herein are methods of selecting a treatment for a subject in need thereof, that include: (a) identifying a subject having resistance to an anti-TNFα agent; and (b) selecting for the identified subject a treatment comprising a therapeutically effective amount of a compound for Formula I or a pharmaceutically acceptable salt, solvate, or co-crystal thereof.

Provided herein are methods of selecting a treatment for a subject in need thereof, that include selecting a treatment comprising a therapeutically effective amount of a compound for Formula I or a pharmaceutically acceptable salt, solvate, or co-crystal thereof for a subject identified as having resistance to an anti-TNFα agent.

In some embodiments of any of the methods described herein, the treatment further includes a therapeutically effective amount of an anti-TNFα agent, in addition to the NLRP3 antagonist.

An “antagonist” of NLRP3 includes compounds that inhibit the ability of NLRP3 to induce the production of IL-1 and/or IL-18 by directly binding to NLRP3, or by inactivating, destabilizing, altering distribution, of NLRP3 or otherwise.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating (e.g., agonizing, partially agonizing, antagonizing) NLRP3 activity are featured that include contacting NLRP3 with a chemical entity described herein (e.g., a compound described generically or specifically herein or a pharmaceutically acceptable salt thereof or compositions containing the same). Methods include in vitro methods, e.g., contacting a sample that includes one or more cells comprising NLRP3, as well as in vivo methods.

In a further aspect, methods of treatment of a disease in which NLRP3 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In a further aspect, methods of treatment are featured that include administering to a subject a chemical entity described herein (e.g., a compound described generically or specifically herein or a pharmaceutically acceptable salt thereof or compositions containing the same), wherein the chemical entity is administered in an amount effective to treat a disease in which NLRP3 signaling contributes to the pathology and/or symptoms and/or progression of the disease, thereby treating the disease.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis, osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP3 molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP3, or a pharmaceutically acceptable salt and/or hydrate and/or cocrystal thereof;) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, saturated or unsaturated, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons unless otherwise noted, such as 3 to 8 carbons, such as 3 to 7 carbons, which may be optionally substituted. Carbocyclic rings may be monocyclic or bicyclic, and when bicyclic, can be fused bicyclic, bridged bicyclic, or spirocyclic. Examples of carbocyclic rings include five-membered, six-membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. When heterocyclic rings are bicyclic or tricyclic, any two connected rings of the bicycle or tricycle may be fused bicyclic, bridged bicyclic, or spirocyclic. Examples of heterocyclic rings include five-membered, six-membered, and seven-membered heterocyclic rings.

The term “cycloalkyl” as used herein includes an nonaromatic cyclic, bicylic, fused, or spiro hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to an nonaromatic 5-8 membered monocyclic, 8-12 membered bicyclic, or 11-14 membered tricyclic ring, fused, or spiro system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocycloalkyls include five-membered, six-membered, and seven-membered heterocycloalkyl rings. Examples include piperazinyl, pyrrolidinyl, dioxanyl, morpholinyl, tetrahydrofuranyl, and the like.

The term “aryl” is intended to mean an aromatic ring radical containing 6 to 10 ring carbons. Examples include phenyl and naphthyl.

The term “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

The term “hydroxy” refers to an OH group.

The term “amino” refers to an NH₂ group.

The term “oxo” refers to O. By way of example, substitution of a CH₂ a group with oxo gives a C═O group.

As used herein, the terms “the ring A” or “A” are used interchangeably to denote

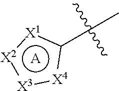

in formulas AA and AA-1, wherein the bond that is shown as being broken by the wavy line [symbol] connects A to the S(O₂)NHC(O)NH moiety of Formula AA.

As used herein, the terms “the ring B” or “B” are used interchangeably to denote

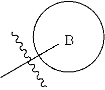

in formula AA wherein the bond that is shown as being broken by the wavy line [symbol] connects B to the NHC(O) group of Formula AA.

As used herein, the terms “the ring B” or “B” are used interchangeably to denote

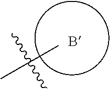

in formula AA-4 wherein the bond that is shown as being broken by the wavy line [symbol] connects B′ to the NHC(O) group of Formula AA-1.

As used herein, the term “the substituted ring B” or “the optionally substituted ring B” is used to denote

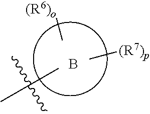

in formula AA and

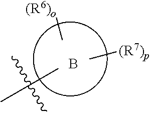

in formula AA-1, wherein the bond that is shown as being broken by the wavy line [symbol] connects B to the NHC(O) group of Formula AA and Formula AA-1.

As used herein, the recitation “S(O₂)”, alone or as part of a larger recitation, refers to the group

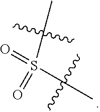 [SMILES: *S(*)(=O)=O]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The scope of the compounds disclosed herein includes tautomeric form of the compounds. Thus, by way of example, a compound that is represented as containing the moiety

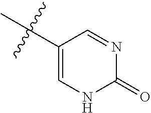 [SMILES: *c1cnc(=O)[nH]c1]

is also intended to include the tautomeric form containing the moiety

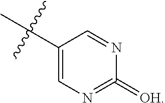

The details of one or more embodiments of the invention are set forth in the accompanying drawings and the description below. Other features and advantages of the invention will be apparent from the description and drawings, and from the claims.

As used herein, the terms “patient” or “subject” refer to a mammalian organism, preferably a human being, who is diseased with the condition (i.e. disease or disorder) of interest and who would benefit from the treatment.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition.

As used herein, the term “therapeutically effective amount” refers to an amount of the compound of the invention, e.g. tropifexor (as herein defined, e.g. in free form or as a stereoisomer, an enantiomer, a pharmaceutically acceptable salt, solvate, prodrug, ester thereof and/or an amino acid conjugate thereof), or cenicriviroc (in free form or as a pharmaceutically acceptable salt, solvate, prodrug, and/or ester thereof, e.g. in free form or as a pharmaceutically acceptable salt thereof), which is sufficient to achieve the stated effect. Accordingly, a therapeutically effective amount used for the treatment or prevention of a liver disease or disorder as hereinabove defined is an amount sufficient for the treatment or prevention of such a disease or disorder.

### DESCRIPTION OF THE DRAWINGS

FIG. 1: Expression levels of RNA encoding NLRP3 in Crohn's Disease patients who are responsive and non-responsive to infliximab.

FIG. 2: Expression levels of RNA encoding IL-1β in Crohn's Disease patients who are responsive and non-responsive to infliximab.

FIG. 3: Expression levels of RNA encoding NLRP3 in Ulcerative Colitis (UC) patients who are responsive and non-responsive to infliximab.

FIG. 4: Expression levels of RNA encoding IL-1β in Ulcerative Colitis (UC) patients who are responsive and non-responsive to infliximab.

### DETAILED DESCRIPTION

In one aspect, provided herein is a compound of Formula AA

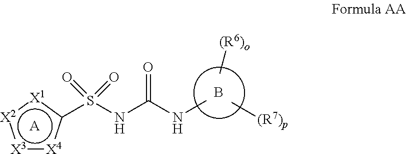

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
when X⁴ is —X⁵—X⁶, then: X¹ is N or CR¹, X² is N or CR², and X³ is N or CR³;
when X⁴ is other than —X⁵—X⁶—, then

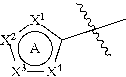

comprises at least one of CR¹, CR², CR³, and CR⁴;
when X⁴ is —X⁵—X⁶—, then

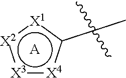

comprises at least two of CR¹, CR², CR³, CR⁵, and CR⁶;
when X⁴ is other than —X⁵—X⁶—, then

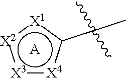

comprises at least one of CR¹, CR², CR³, and CR⁴;
when X⁴ is —X⁵—X⁶—, then

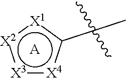

comprises at least two of CR¹, CR², CR³, CR⁵, and CR⁶;
from two to four of R¹, R², R³, and R⁴ are present or from two to five of R¹, R², R³, R⁵, and R⁶ are present; and
wherein at least two of the two to four R¹, R², R³, and R⁴ or at least two of the two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰;

R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹; or

at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;

R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
wherein any of R¹, R², R³, R⁴, R⁵, and R⁶ that are not taken together with the atoms connecting them to form a ring, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;
R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;
R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
B is a 5-10-membered heteroaryl or C₆-C₁₀ aryl ring;
o=1 or 2;
p=0, 1, 2, or 3;
R⁶ and R⁷ are each independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
provided that when B is 5-10-membered heteroaryl including from 2-3 ring nitrogen atoms, at least one R⁶ is attached to B at a position ortho to the —HNC(═O)NHS(O)₂— moiety of Formula AA;
when B is 2-pyridyl, pyrimidin-6-yl, or pyrimidin-4-yl, B is not substituted with a cyano group at a position ortho to the —HNC(═O)NHS(O)₂— moiety of Formula AA;
and
with the proviso that the compound of Formula AA is not a compound selected from the group consisting of:

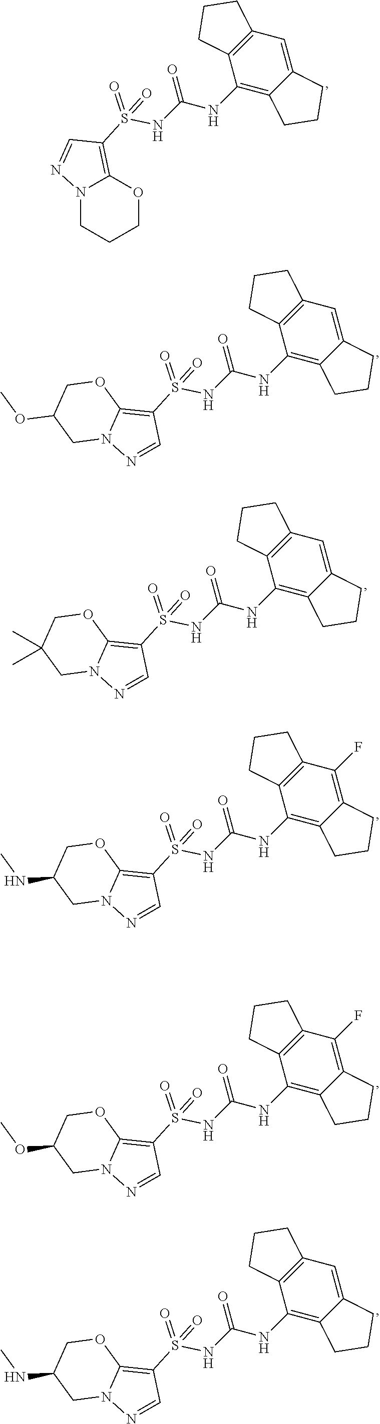 [SMILES: O=C(Nc1c2c(cc3c1CCC3)CCC2)NS(=O)(=O)c1cnn2c1OCCC2 ; COC1COc2c(S(=O)(=O)NC(=O)Nc3c4c(cc5c3CCC5)CCC4)cnn2C1 ; CC1(C)COc2c(S(=O)(=O)NC(=O)Nc3c4c(cc5c3CCC5)CCC4)cnn2C1 ; CN[C@H]1COc2c(S(=O)(=O)NC(=O)Nc3c4c(c(F)c5c3CCC5)CCC4)cnn2C1 ; CO[C@H]1COc2c(S(=O)(=O)NC(=O)Nc3c4c(c(F)c5c3CCC5)CCC4)cnn2C1 ; CN[C@H]1COc2c(S(=O)(=O)NC(=O)Nc3c4c(cc5c3CCC5)CCC4)cnn2C1]

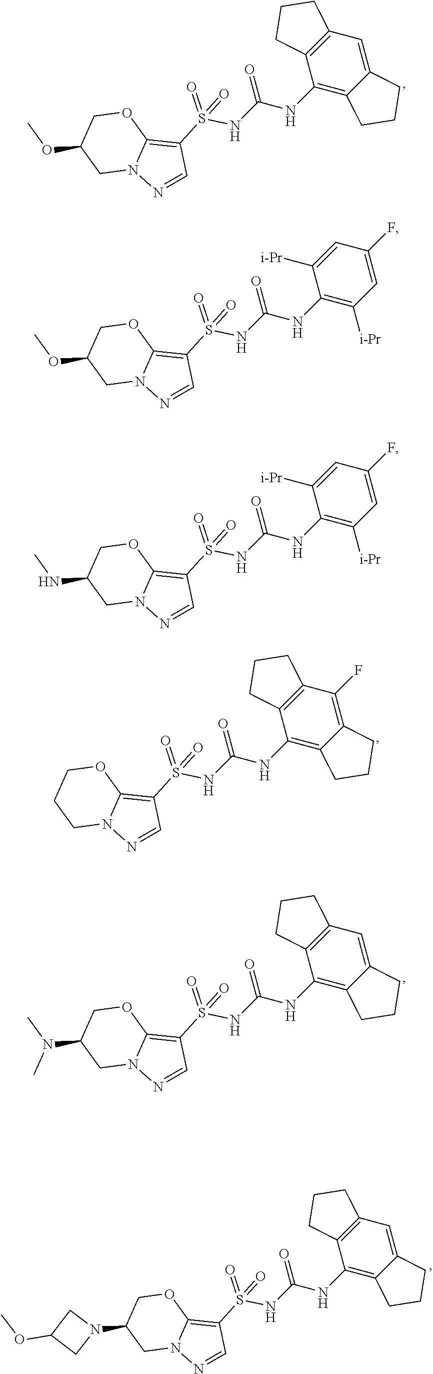 [SMILES: CO[C@H]1COc2c(S(=O)(=O)NC(=O)Nc3c4c(cc5c3CCC5)CCC4)cnn2C1 ; CO[C@H]1COc2c(S(=O)(=O)NC(=O)Nc3c(C(C)C)cc(F)cc3C(C)C)cnn2C1 ; CN[C@H]1COc2c(S(=O)(=O)NC(=O)Nc3c(C(C)C)cc(F)cc3C(C)C)cnn2C1 ; O=C(Nc1c2c(c(F)c3c1CCC3)CCC2)NS(=O)(=O)c1cnn2c1OCCC2 ; CN(C)[C@H]1COc2c(S(=O)(=O)NC(=O)Nc3c4c(cc5c3CCC5)CCC4)cnn2C1 ; COC1CN([C@H]2COc3c(S(=O)(=O)NC(=O)Nc4c5c(cc6c4CCC6)CCC5)cnn3C2)C1]

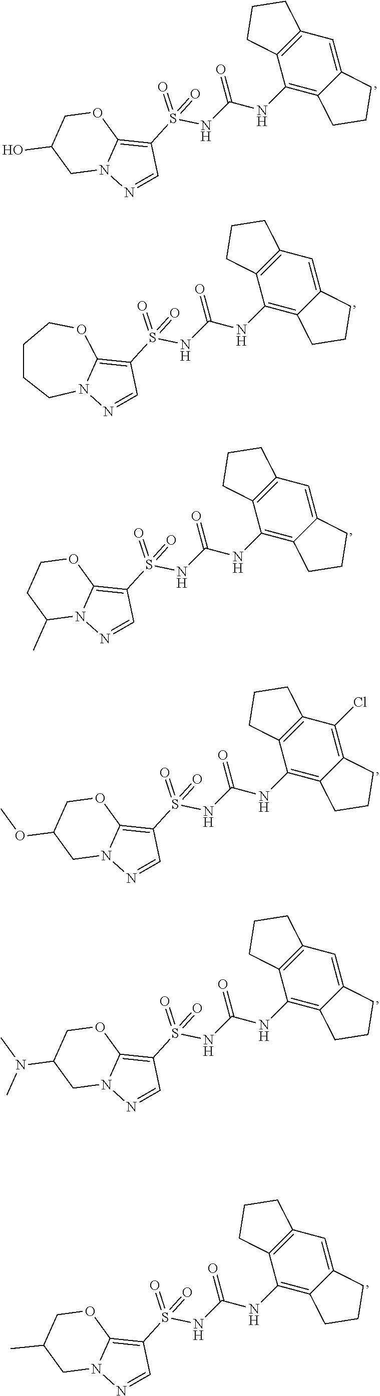 [SMILES: O=C(Nc1c2c(cc3c1CCC3)CCC2)NS(=O)(=O)c1cnn2c1OCC(O)C2 ; O=C(Nc1c2c(cc3c1CCC3)CCC2)NS(=O)(=O)c1cnn2c1OCCCC2 ; CC1CCOc2c(S(=O)(=O)NC(=O)Nc3c4c(cc5c3CCC5)CCC4)cnn21 ; COC1COc2c(S(=O)(=O)NC(=O)Nc3c4c(c(Cl)c5c3CCC5)CCC4)cnn2C1 ; CN(C)C1COc2c(S(=O)(=O)NC(=O)Nc3c4c(cc5c3CCC5)CCC4)cnn2C1 ; CC1COc2c(S(=O)(=O)NC(=O)Nc3c4c(cc5c3CCC5)CCC4)cnn2C1]

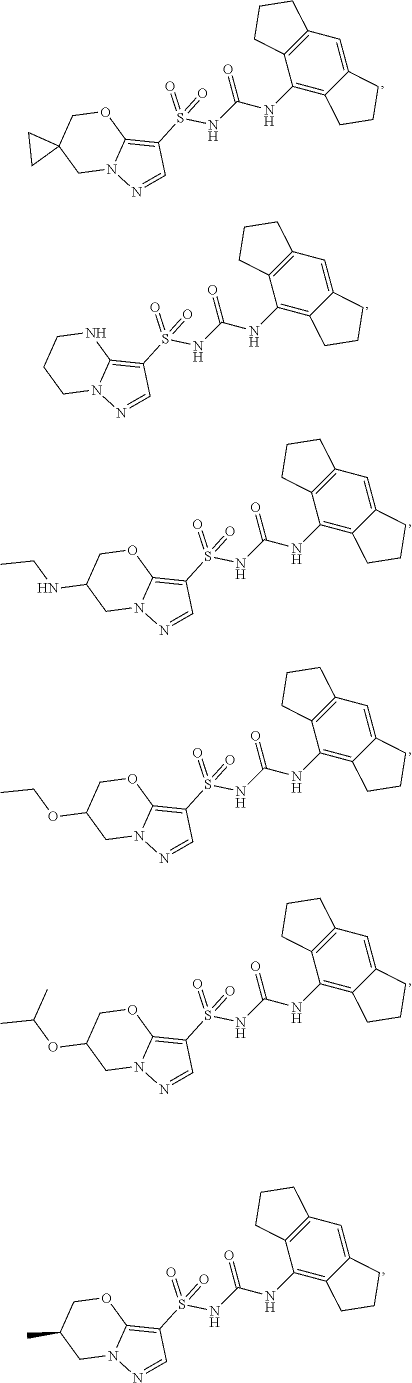 [SMILES: O=C(Nc1c2c(cc3c1CCC3)CCC2)NS(=O)(=O)c1cnn2c1OCC1(CC1)C2 ; O=C(Nc1c2c(cc3c1CCC3)CCC2)NS(=O)(=O)c1cnn2c1NCCC2 ; CCNC1COc2c(S(=O)(=O)NC(=O)Nc3c4c(cc5c3CCC5)CCC4)cnn2C1 ; CCOC1COc2c(S(=O)(=O)NC(=O)Nc3c4c(cc5c3CCC5)CCC4)cnn2C1 ; CC(C)OC1COc2c(S(=O)(=O)NC(=O)Nc3c4c(cc5c3CCC5)CCC4)cnn2C1 ; C[C@H]1COc2c(S(=O)(=O)NC(=O)Nc3c4c(cc5c3CCC5)CCC4)cnn2C1]

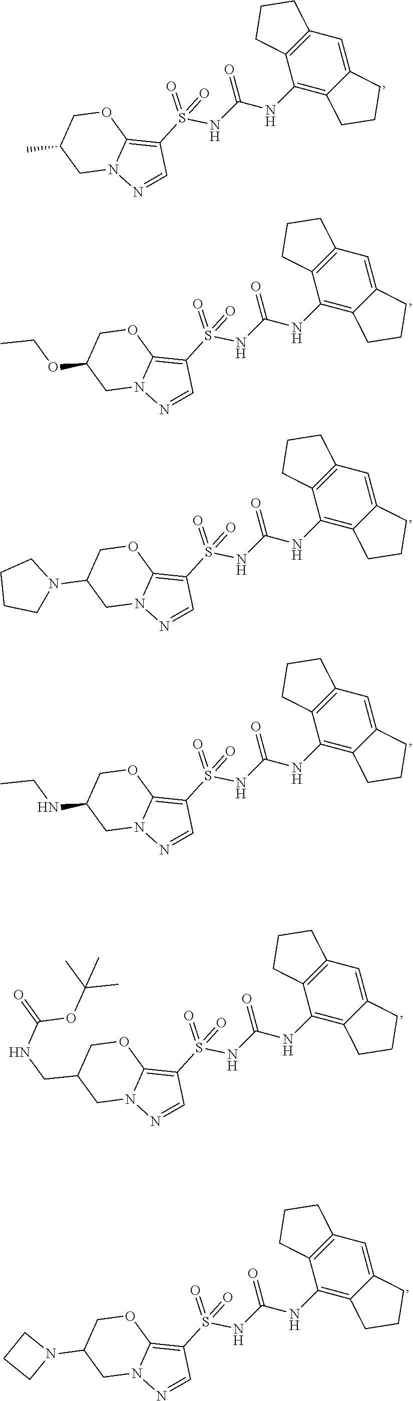 [SMILES: C[C@@H]1COc2c(S(=O)(=O)NC(=O)Nc3c4c(cc5c3CCC5)CCC4)cnn2C1 ; CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCCC1)C2 ; CCN[C@H]1COc2c(S(=O)(=O)NC(=O)Nc3c4c(cc5c3CCC5)CCC4)cnn2C1 ; CC(C)(C)OC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCC1)C2]

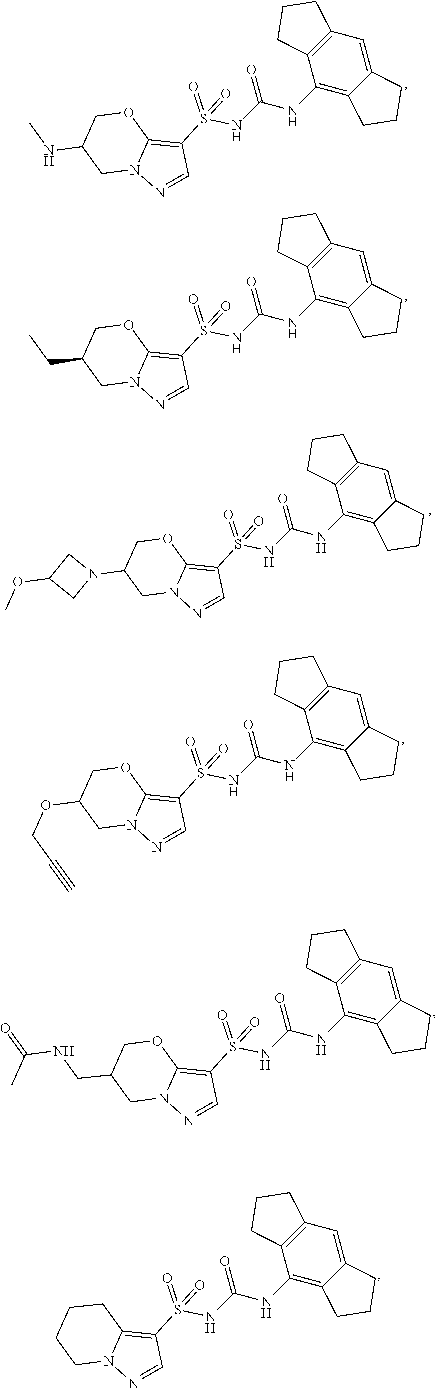 [SMILES: CNC1COc2c(S(=O)(=O)NC(=O)Nc3c4c(cc5c3CCC5)CCC4)cnn2C1 ; CC[C@H]1COc2c(S(=O)(=O)NC(=O)Nc3c4c(cc5c3CCC5)CCC4)cnn2C1 ; COC1CN(C2COc3c(S(=O)(=O)NC(=O)Nc4c5c(cc6c4CCC6)CCC5)cnn3C2)C1 ; C#CCOC1COc2c(S(=O)(=O)NC(=O)Nc3c4c(cc5c3CCC5)CCC4)cnn2C1 ; CC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1CCCC2]

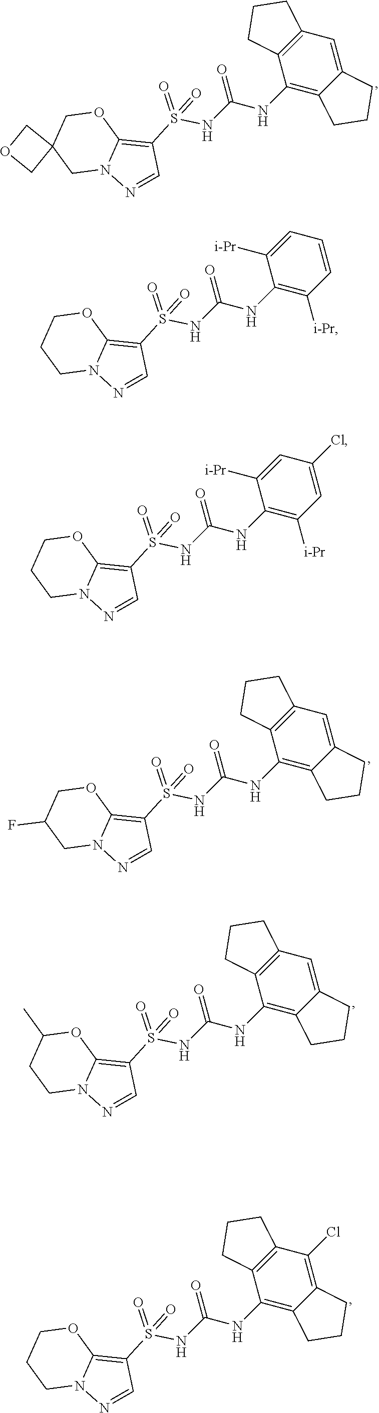 [SMILES: O=C(Nc1c2c(cc3c1CCC3)CCC2)NS(=O)(=O)c1cnn2c1OCC1(COC1)C2 ; CC(C)c1cccc(C(C)C)c1NC(=O)NS(=O)(=O)c1cnn2c1OCCC2 ; CC(C)c1cc(Cl)cc(C(C)C)c1NC(=O)NS(=O)(=O)c1cnn2c1OCCC2 ; O=C(Nc1c2c(cc3c1CCC3)CCC2)NS(=O)(=O)c1cnn2c1OCC(F)C2 ; CC1CCn2ncc(S(=O)(=O)NC(=O)Nc3c4c(cc5c3CCC5)CCC4)c2O1 ; O=C(Nc1c2c(c(Cl)c3c1CCC3)CCC2)NS(=O)(=O)c1cnn2c1OCCC2]

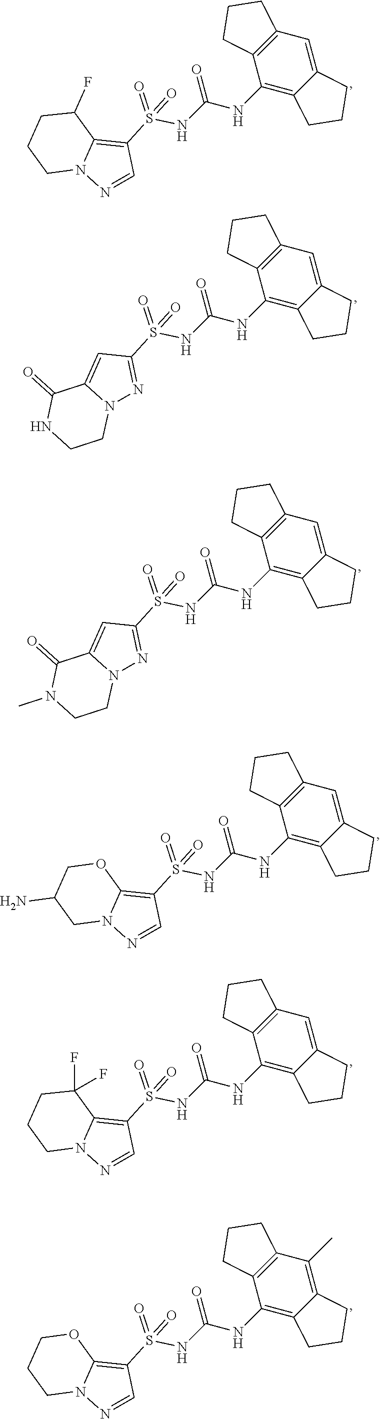 [SMILES: O=C(Nc1c2c(cc3c1CCC3)CCC2)NS(=O)(=O)c1cnn2c1C(F)CCC2 ; O=C(Nc1c2c(cc3c1CCC3)CCC2)NS(=O)(=O)c1cc2n(n1)CCNC2=O ; CN1CCn2nc(S(=O)(=O)NC(=O)Nc3c4c(cc5c3CCC5)CCC4)cc2C1=O ; NC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1C(F)(F)CCC2 ; Cc1c2c(c(NC(=O)NS(=O)(=O)c3cnn4c3OCCC4)c3c1CCC3)CCC2]

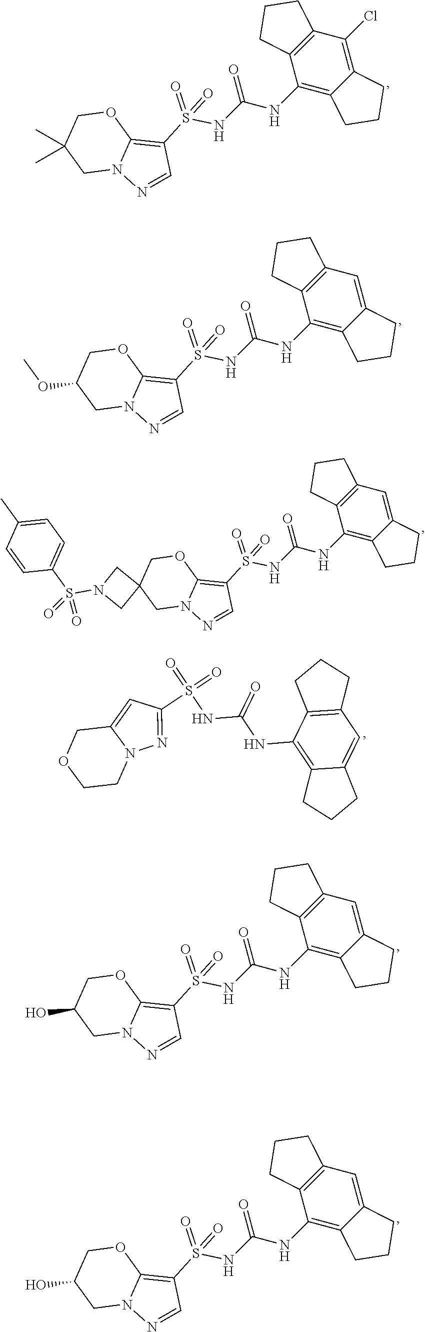 [SMILES: CC1(C)COc2c(S(=O)(=O)NC(=O)Nc3c4c(c(Cl)c5c3CCC5)CCC4)cnn2C1 ; CO[C@@H]1COc2c(S(=O)(=O)NC(=O)Nc3c4c(cc5c3CCC5)CCC4)cnn2C1 ; Cc1ccc(S(=O)(=O)N2CC3(COc4c(S(=O)(=O)NC(=O)Nc5c6c(cc7c5CCC7)CCC6)cnn4C3)C2)cc1 ; O=C(Nc1c2c(cc3c1CCC3)CCC2)NS(=O)(=O)c1cc2n(n1)CCOC2 ; O=C(Nc1c2c(cc3c1CCC3)CCC2)NS(=O)(=O)c1cnn2c1OC[C@H](O)C2 ; O=C(Nc1c2c(cc3c1CCC3)CCC2)NS(=O)(=O)c1cnn2c1OC[C@@H](O)C2]

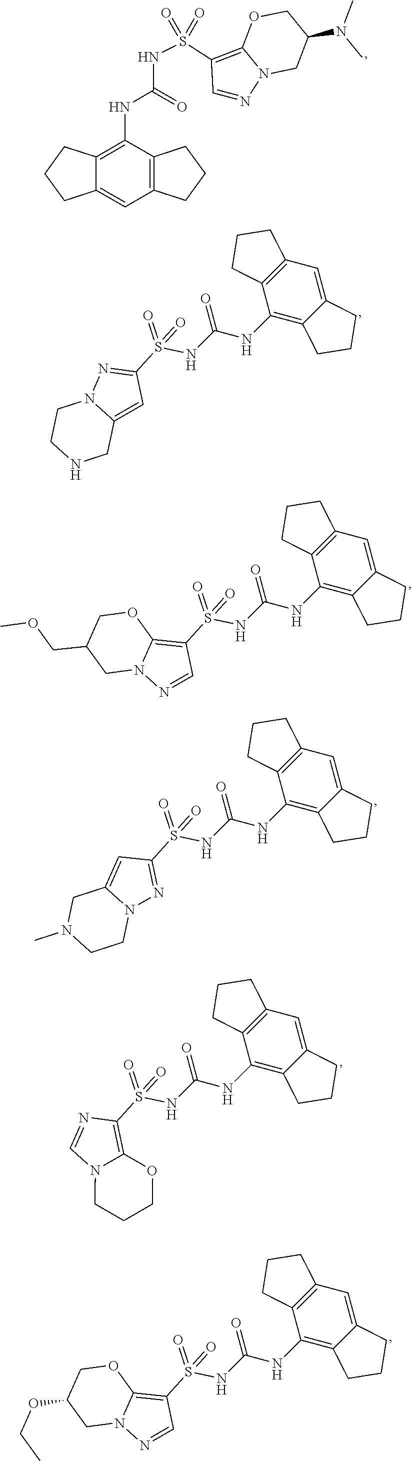

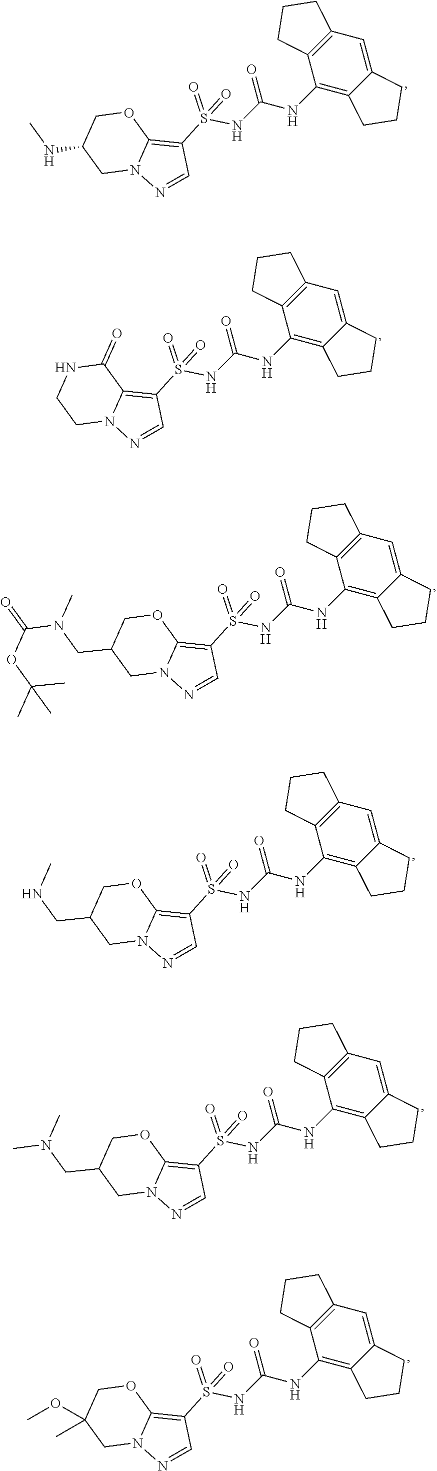 [SMILES: CN[C@@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1C(=O)NCC2 ; CN(CC1COc2c(S(=O)(=O)NC(=O)Nc3c4c(cc5c3CCC5)CCC4)cnn2C1)C(=O)OC(C)(C)C ; CNCC1COc2c(S(=O)(=O)NC(=O)Nc3c4c(cc5c3CCC5)CCC4)cnn2C1 ; CN(C)CC1COc2c(S(=O)(=O)NC(=O)Nc3c4c(cc5c3CCC5)CCC4)cnn2C1 ; COC1(C)COc2c(S(=O)(=O)NC(=O)Nc3c4c(cc5c3CCC5)CCC4)cnn2C1]

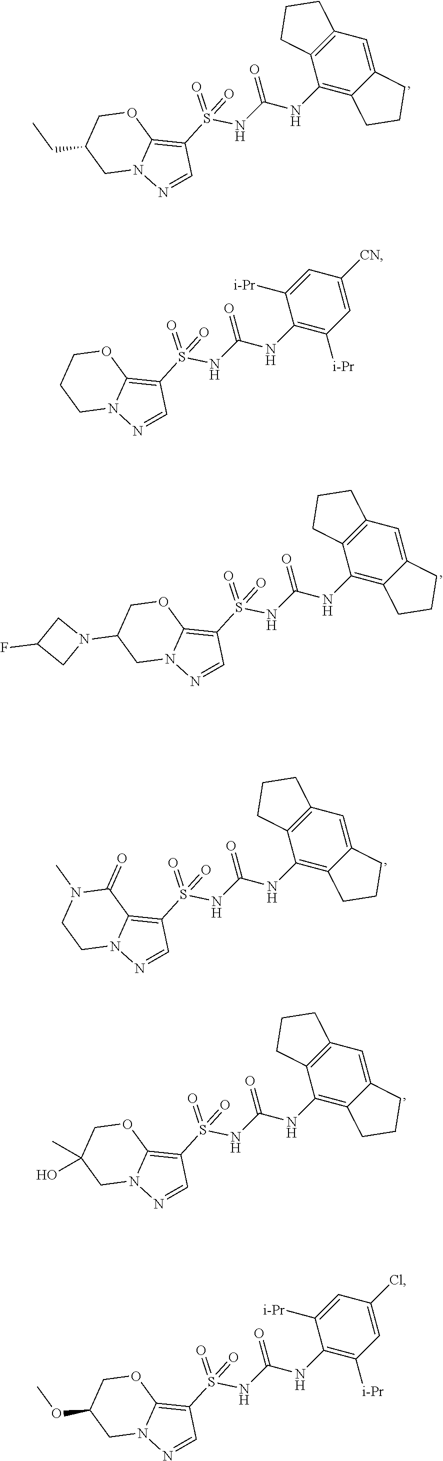 [SMILES: CC[C@@H]1COc2c(S(=O)(=O)NC(=O)Nc3c4c(cc5c3CCC5)CCC4)cnn2C1 ; CC(C)c1cc(C#N)cc(C(C)C)c1NC(=O)NS(=O)(=O)c1cnn2c1OCCC2 ; O=C(Nc1c2c(cc3c1CCC3)CCC2)NS(=O)(=O)c1cnn2c1OCC(N1CC(F)C1)C2 ; CN1CCn2ncc(S(=O)(=O)NC(=O)Nc3c4c(cc5c3CCC5)CCC4)c2C1=O ; CC1(O)COc2c(S(=O)(=O)NC(=O)Nc3c4c(cc5c3CCC5)CCC4)cnn2C1 ; CO[C@H]1COc2c(S(=O)(=O)NC(=O)Nc3c(C(C)C)cc(Cl)cc3C(C)C)cnn2C1]

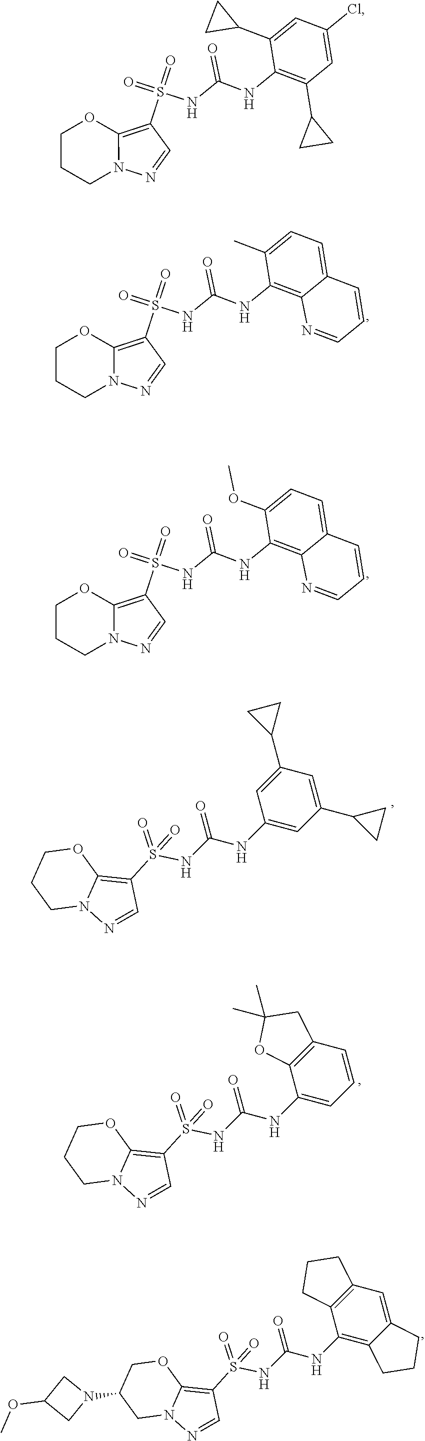 [SMILES: O=C(Nc1c(C2CC2)cc(Cl)cc1C1CC1)NS(=O)(=O)c1cnn2c1OCCC2 ; Cc1ccc2cccnc2c1NC(=O)NS(=O)(=O)c1cnn2c1OCCC2 ; COc1ccc2cccnc2c1NC(=O)NS(=O)(=O)c1cnn2c1OCCC2 ; O=C(Nc1cc(C2CC2)cc(C2CC2)c1)NS(=O)(=O)c1cnn2c1OCCC2 ; CC1(C)Cc2cccc(NC(=O)NS(=O)(=O)c3cnn4c3OCCC4)c2O1 ; COC1CN([C@@H]2COc3c(S(=O)(=O)NC(=O)Nc4c5c(cc6c4CCC6)CCC5)cnn3C2)C1]

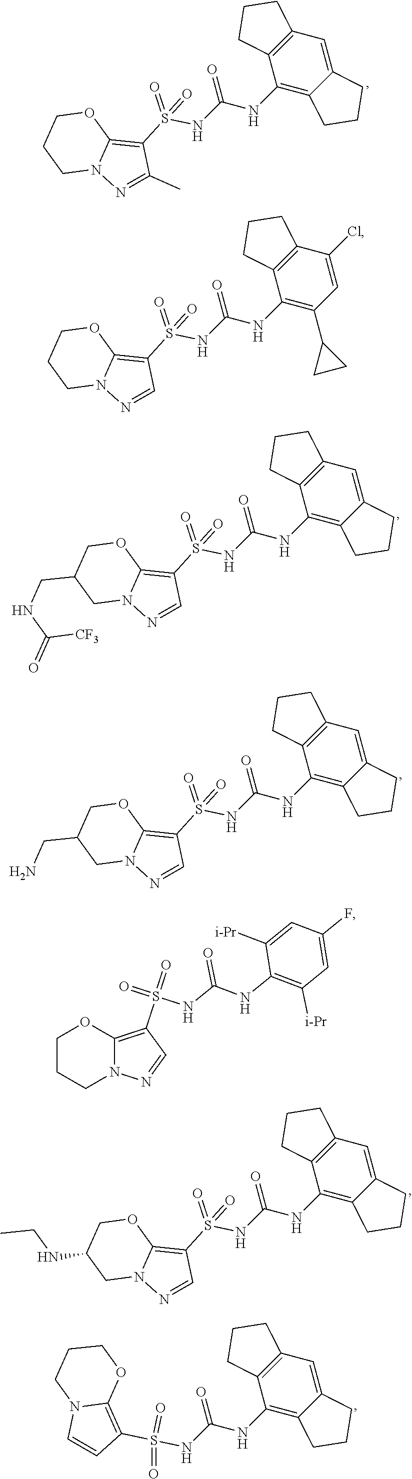

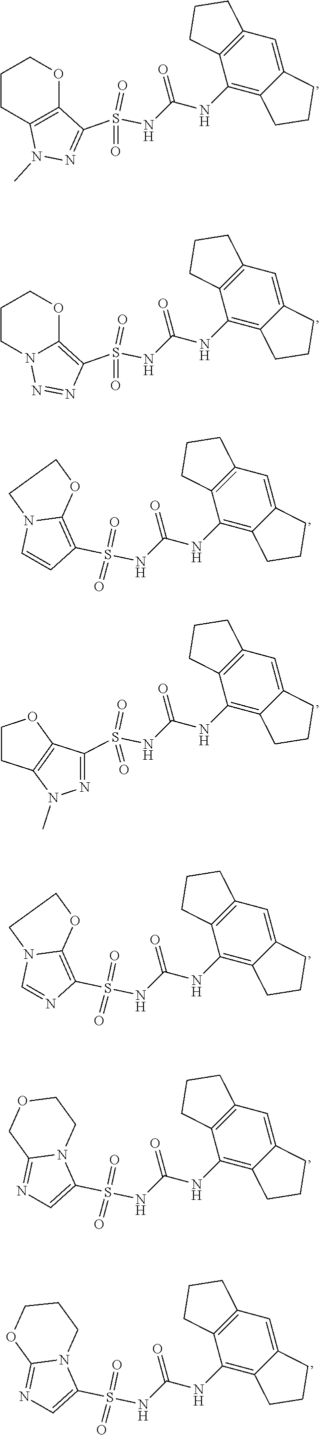

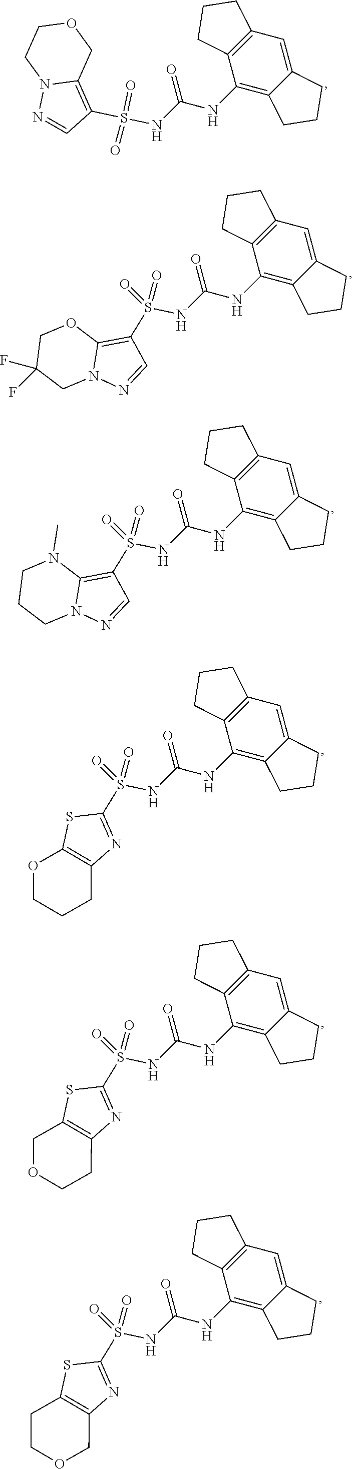 [SMILES: O=C(Nc1c2c(cc3c1CCC3)CCC2)NS(=O)(=O)c1cnn2c1COCC2 ; O=C(Nc1c2c(cc3c1CCC3)CCC2)NS(=O)(=O)c1cnn2c1OCC(F)(F)C2 ; CN1CCCn2ncc(S(=O)(=O)NC(=O)Nc3c4c(cc5c3CCC5)CCC4)c21 ; O=C(Nc1c2c(cc3c1CCC3)CCC2)NS(=O)(=O)c1nc2c(s1)OCCC2 ; O=C(Nc1c2c(cc3c1CCC3)CCC2)NS(=O)(=O)c1nc2c(s1)COCC2 ; O=C(Nc1c2c(cc3c1CCC3)CCC2)NS(=O)(=O)c1nc2c(s1)CCOC2]

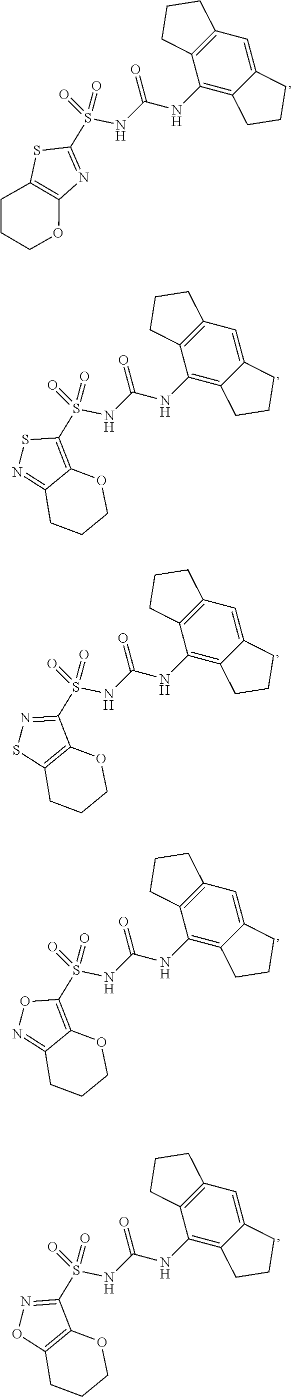 [SMILES: O=C(Nc1c2c(cc3c1CCC3)CCC2)NS(=O)(=O)c1nc2c(s1)CCCO2 ; O=C(Nc1c2c(cc3c1CCC3)CCC2)NS(=O)(=O)c1snc2c1OCCC2 ; O=C(Nc1c2c(cc3c1CCC3)CCC2)NS(=O)(=O)c1nsc2c1OCCC2 ; O=C(Nc1c2c(cc3c1CCC3)CCC2)NS(=O)(=O)c1onc2c1OCCC2 ; O=C(Nc1c2c(cc3c1CCC3)CCC2)NS(=O)(=O)c1noc2c1OCCC2]

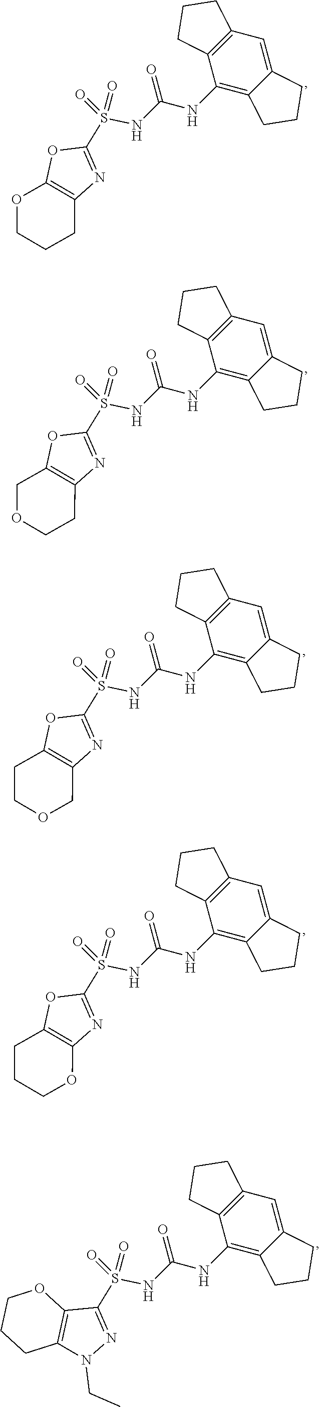 [SMILES: O=C(Nc1c2c(cc3c1CCC3)CCC2)NS(=O)(=O)c1nc2c(o1)OCCC2 ; O=C(Nc1c2c(cc3c1CCC3)CCC2)NS(=O)(=O)c1nc2c(o1)COCC2 ; O=C(Nc1c2c(cc3c1CCC3)CCC2)NS(=O)(=O)c1nc2c(o1)CCOC2 ; O=C(Nc1c2c(cc3c1CCC3)CCC2)NS(=O)(=O)c1nc2c(o1)CCCO2 ; CCn1nc(S(=O)(=O)NC(=O)Nc2c3c(cc4c2CCC4)CCC3)c2c1CCCO2]

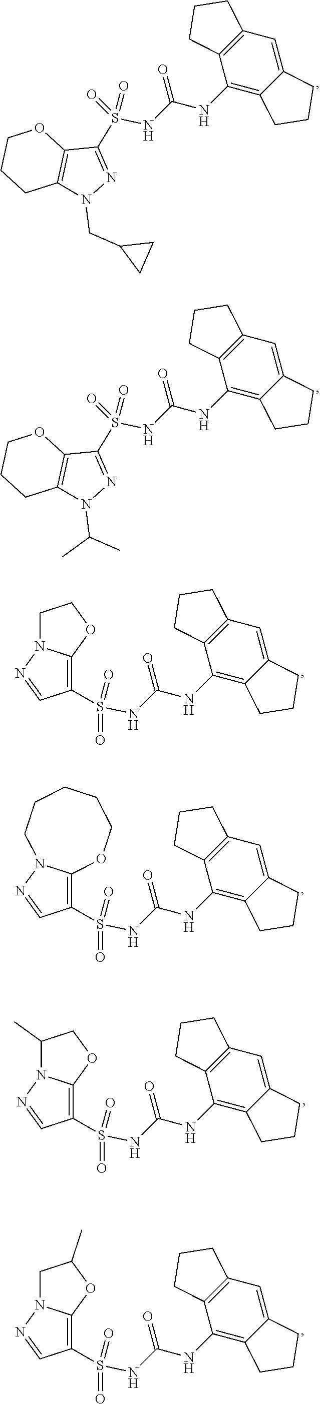 [SMILES: O=C(Nc1c2c(cc3c1CCC3)CCC2)NS(=O)(=O)c1nn(CC2CC2)c2c1OCCC2 ; CC(C)n1nc(S(=O)(=O)NC(=O)Nc2c3c(cc4c2CCC4)CCC3)c2c1CCCO2 ; O=C(Nc1c2c(cc3c1CCC3)CCC2)NS(=O)(=O)c1cnn2c1OCC2 ; O=C(Nc1c2c(cc3c1CCC3)CCC2)NS(=O)(=O)c1cnn2c1OCCCCC2 ; CC1COc2c(S(=O)(=O)NC(=O)Nc3c4c(cc5c3CCC5)CCC4)cnn21 ; CC1Cn2ncc(S(=O)(=O)NC(=O)Nc3c4c(cc5c3CCC5)CCC4)c2O1]

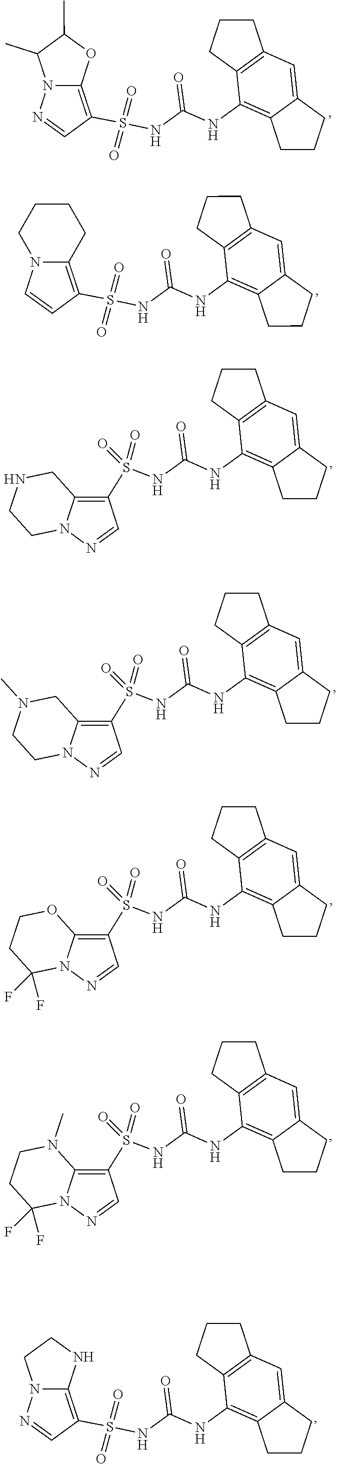

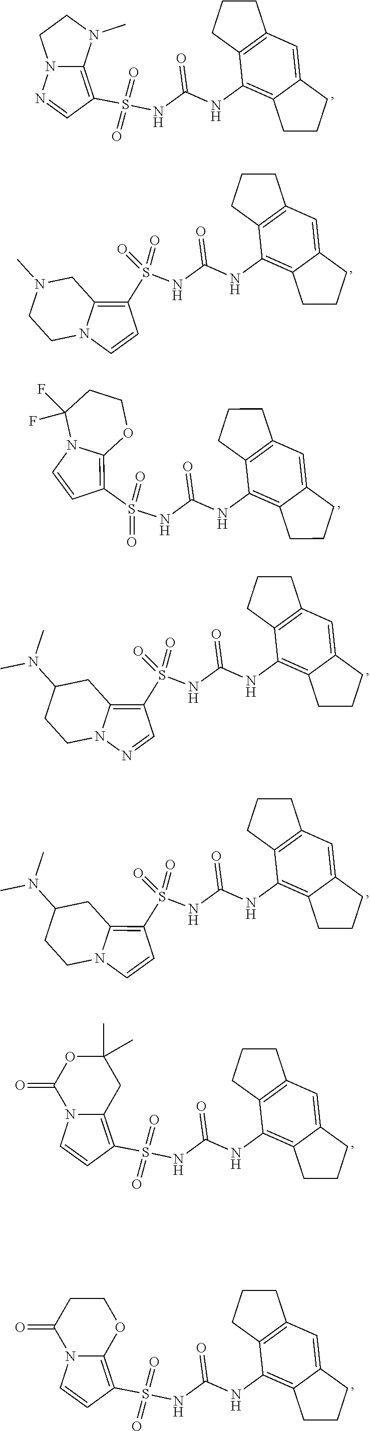

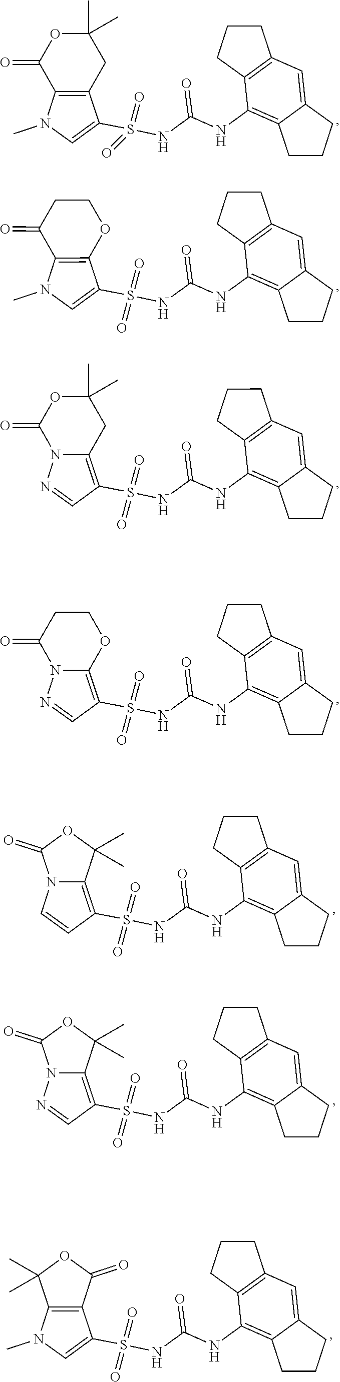

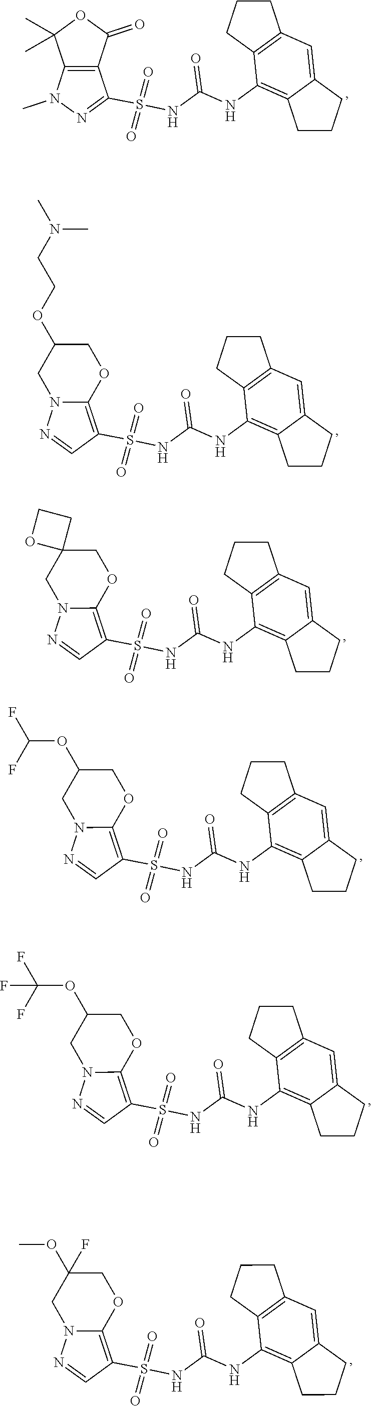 [SMILES: Cn1nc(S(=O)(=O)NC(=O)Nc2c3c(cc4c2CCC4)CCC3)c2c1C(C)(C)OC2=O ; CN(C)CCOC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC1(CCO1)C2 ; O=C(Nc1c2c(cc3c1CCC3)CCC2)NS(=O)(=O)c1cnn2c1OCC(OC(F)F)C2 ; O=C(Nc1c2c(cc3c1CCC3)CCC2)NS(=O)(=O)c1cnn2c1OCC(OC(F)(F)F)C2 ; COC1(F)COc2c(S(=O)(=O)NC(=O)Nc3c4c(cc5c3CCC5)CCC4)cnn2C1]

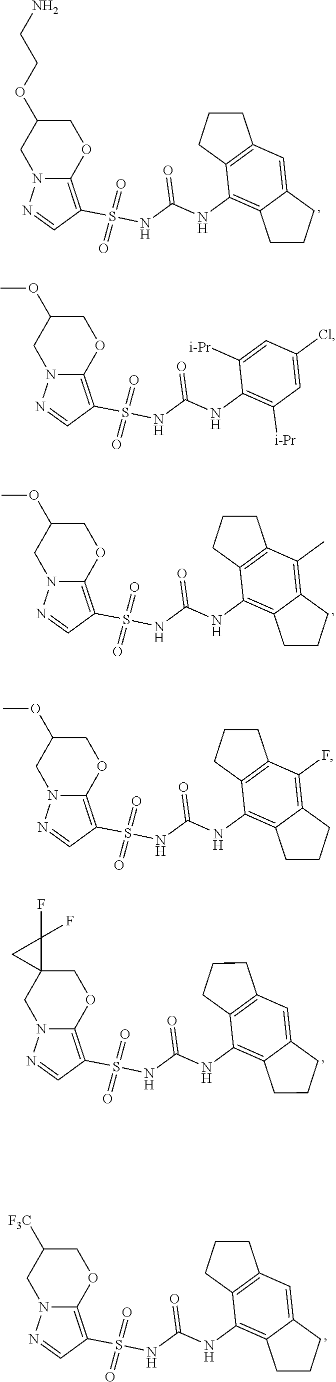 [SMILES: NCCOC1COc2c(S(=O)(=O)NC(=O)Nc3c4c(cc5c3CCC5)CCC4)cnn2C1 ; COC1COc2c(S(=O)(=O)NC(=O)Nc3c(C(C)C)cc(Cl)cc3C(C)C)cnn2C1 ; COC1COc2c(S(=O)(=O)NC(=O)Nc3c4c(c(C)c5c3CCC5)CCC4)cnn2C1 ; COC1COc2c(S(=O)(=O)NC(=O)Nc3c4c(c(F)c5c3CCC5)CCC4)cnn2C1 ; O=C(Nc1c2c(cc3c1CCC3)CCC2)NS(=O)(=O)c1cnn2c1OCC1(C2)CC1(F)F ; O=C(Nc1c2c(cc3c1CCC3)CCC2)NS(=O)(=O)c1cnn2c1OCC(C(F)(F)F)C2]

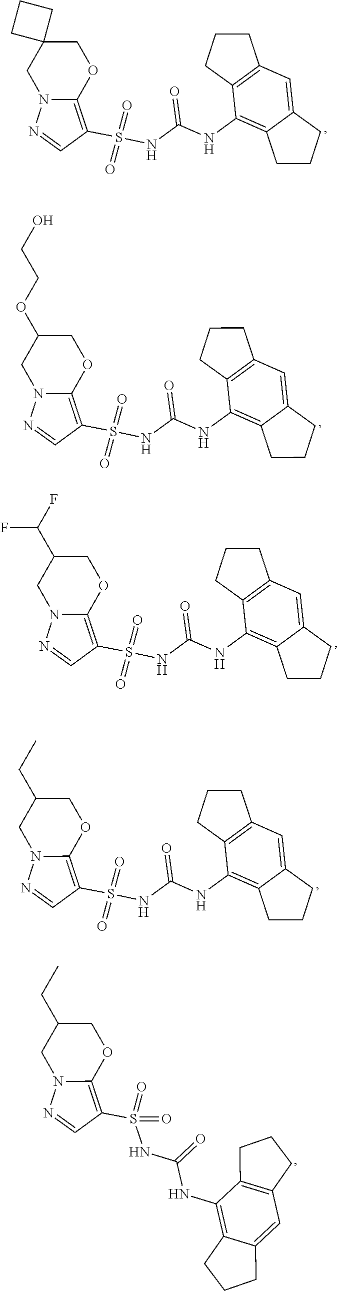 [SMILES: O=C(Nc1c2c(cc3c1CCC3)CCC2)NS(=O)(=O)c1cnn2c1OCC1(CCC1)C2 ; O=C(Nc1c2c(cc3c1CCC3)CCC2)NS(=O)(=O)c1cnn2c1OCC(OCCO)C2 ; O=C(Nc1c2c(cc3c1CCC3)CCC2)NS(=O)(=O)c1cnn2c1OCC(C(F)F)C2 ; CCC1COc2c(S(=O)(=O)NC(=O)Nc3c4c(cc5c3CCC5)CCC4)cnn2C1 ; CCC1COc2c(S(=O)(=O)NC(=O)Nc3c4c(cc5c3CCC5)CCC4)cnn2C1]

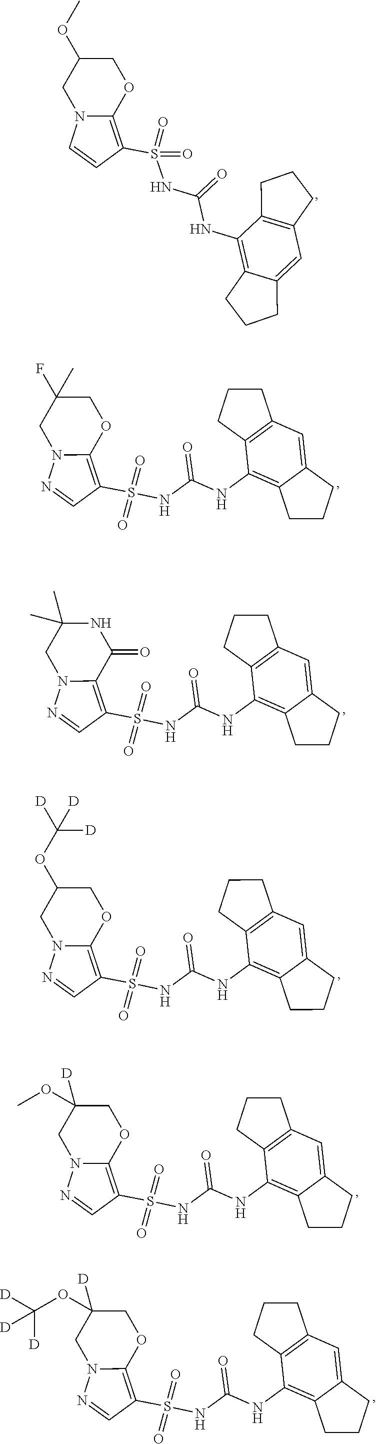 [SMILES: COC1COc2c(S(=O)(=O)NC(=O)Nc3c4c(cc5c3CCC5)CCC4)ccn2C1 ; CC1(F)COc2c(S(=O)(=O)NC(=O)Nc3c4c(cc5c3CCC5)CCC4)cnn2C1 ; CC1(C)Cn2ncc(S(=O)(=O)NC(=O)Nc3c4c(cc5c3CCC5)CCC4)c2C(=O)N1 ; [2H]C([2H])([2H])OC1COc2c(S(=O)(=O)NC(=O)Nc3c4c(cc5c3CCC5)CCC4)cnn2C1 ; [2H]C1(OC)COc2c(S(=O)(=O)NC(=O)Nc3c4c(cc5c3CCC5)CCC4)cnn2C1 ; [2H]C([2H])([2H])OC1([2H])COc2c(S(=O)(=O)NC(=O)Nc3c4c(cc5c3CCC5)CCC4)cnn2C1]

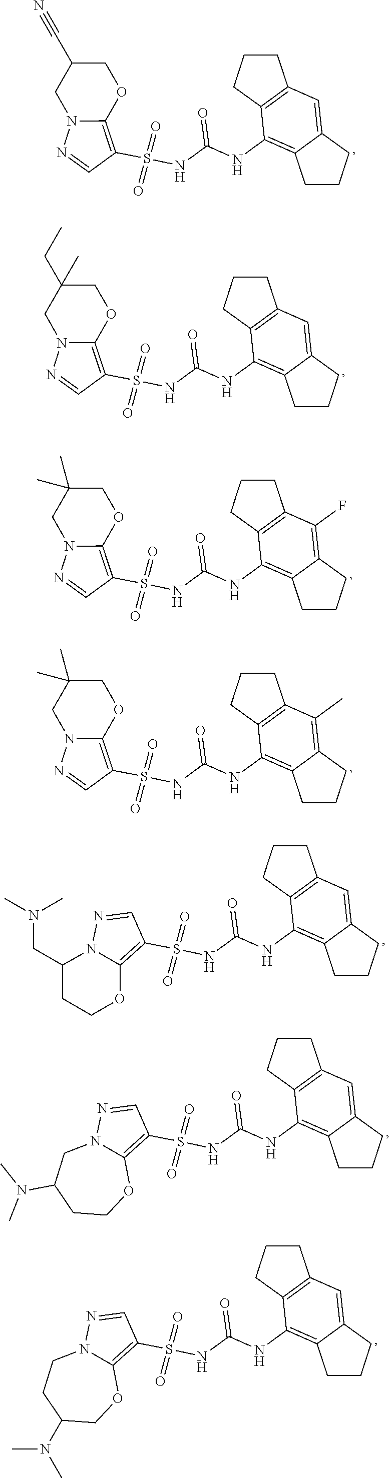

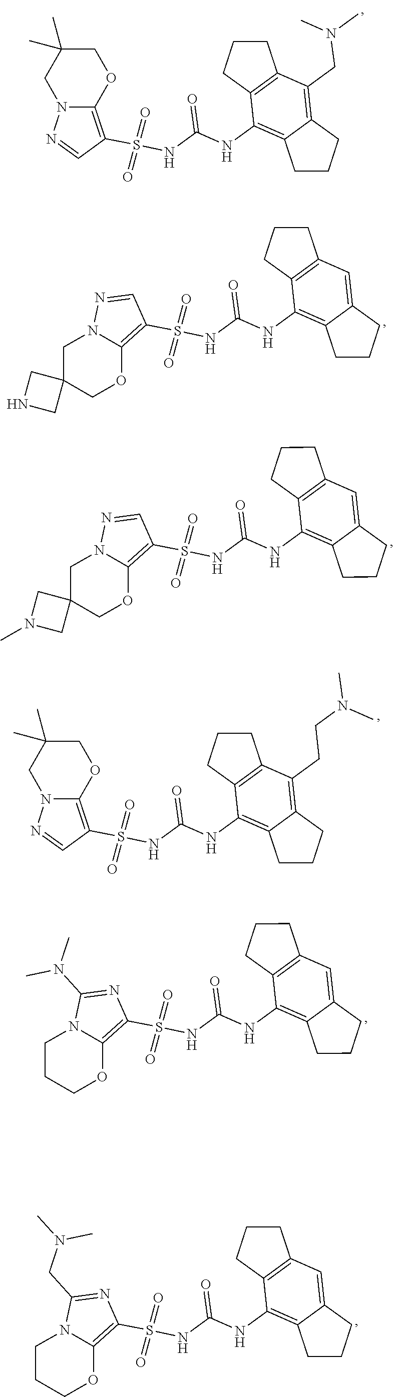

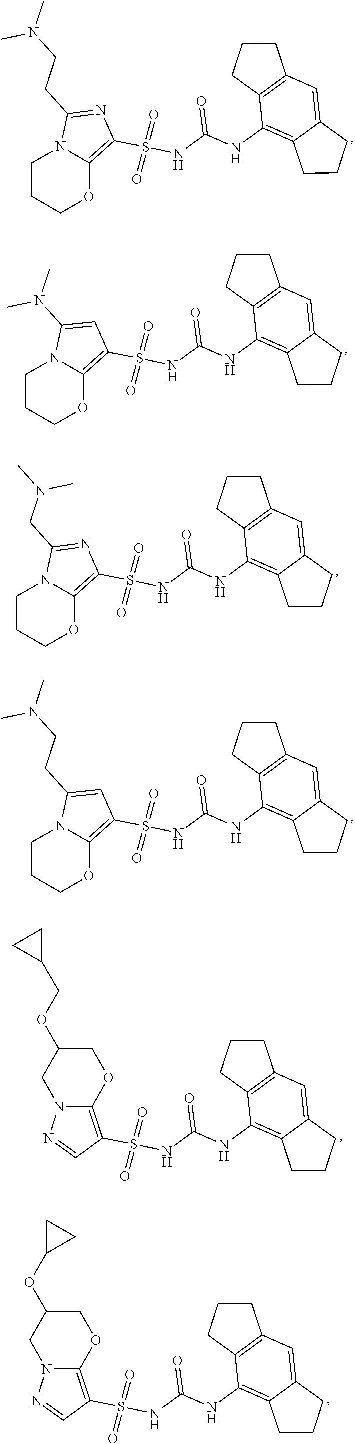

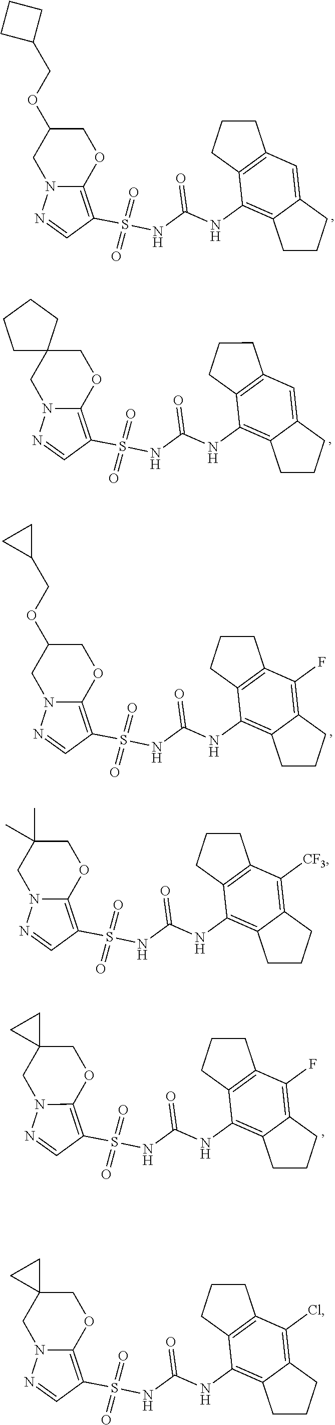 [SMILES: O=C(Nc1c2c(cc3c1CCC3)CCC2)NS(=O)(=O)c1cnn2c1OCC(OCC1CCC1)C2 ; O=C(Nc1c2c(cc3c1CCC3)CCC2)NS(=O)(=O)c1cnn2c1OCC1(CCCC1)C2 ; O=C(Nc1c2c(c(F)c3c1CCC3)CCC2)NS(=O)(=O)c1cnn2c1OCC(OCC1CC1)C2 ; CC1(C)COc2c(S(=O)(=O)NC(=O)Nc3c4c(c(C(F)(F)F)c5c3CCC5)CCC4)cnn2C1 ; O=C(Nc1c2c(c(F)c3c1CCC3)CCC2)NS(=O)(=O)c1cnn2c1OCC1(CC1)C2 ; O=C(Nc1c2c(c(Cl)c3c1CCC3)CCC2)NS(=O)(=O)c1cnn2c1OCC1(CC1)C2]

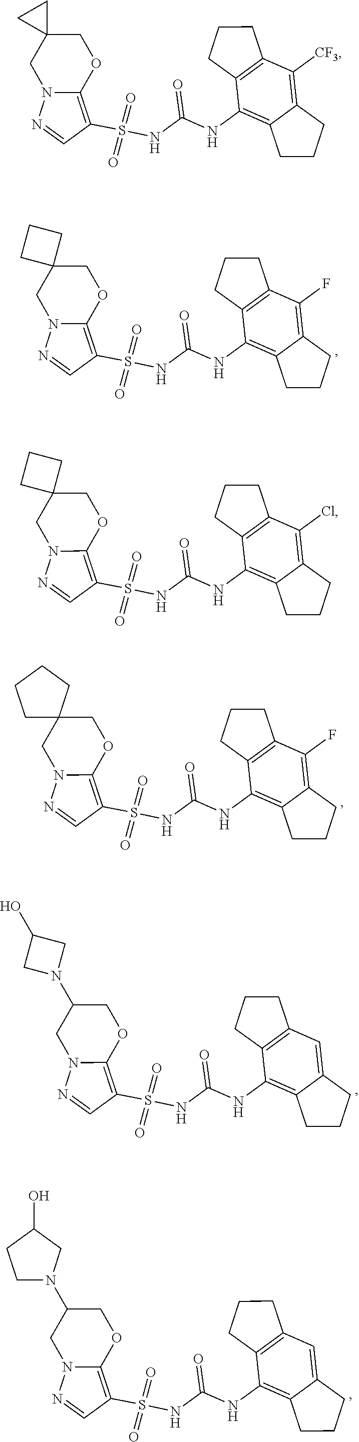 [SMILES: O=C(Nc1c2c(c(C(F)(F)F)c3c1CCC3)CCC2)NS(=O)(=O)c1cnn2c1OCC1(CC1)C2 ; O=C(Nc1c2c(c(F)c3c1CCC3)CCC2)NS(=O)(=O)c1cnn2c1OCC1(CCC1)C2 ; O=C(Nc1c2c(c(Cl)c3c1CCC3)CCC2)NS(=O)(=O)c1cnn2c1OCC1(CCC1)C2 ; O=C(Nc1c2c(c(F)c3c1CCC3)CCC2)NS(=O)(=O)c1cnn2c1OCC1(CCCC1)C2 ; O=C(Nc1c2c(cc3c1CCC3)CCC2)NS(=O)(=O)c1cnn2c1OCC(N1CC(O)C1)C2 ; O=C(Nc1c2c(cc3c1CCC3)CCC2)NS(=O)(=O)c1cnn2c1OCC(N1CCC(O)C1)C2]

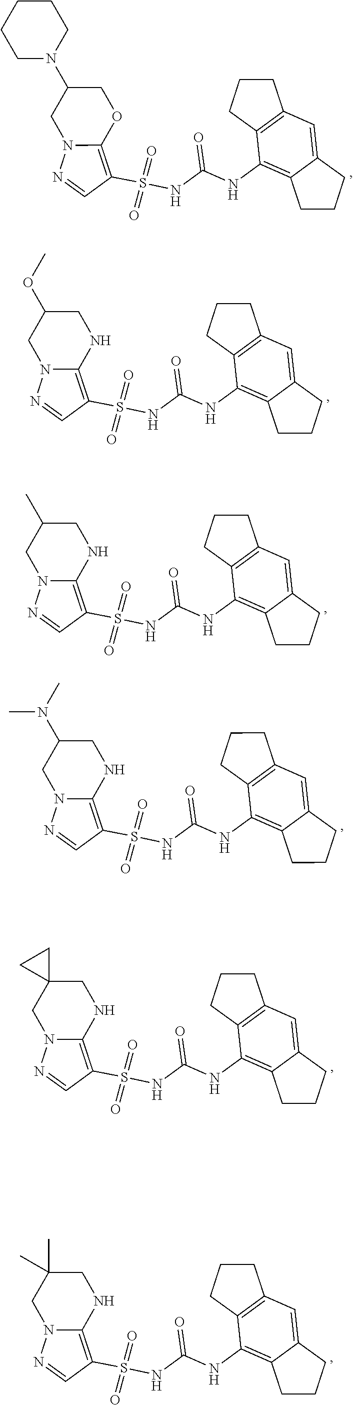 [SMILES: O=C(Nc1c2c(cc3c1CCC3)CCC2)NS(=O)(=O)c1cnn2c1OCC(N1CCCCC1)C2 ; COC1CNc2c(S(=O)(=O)NC(=O)Nc3c4c(cc5c3CCC5)CCC4)cnn2C1 ; CC1CNc2c(S(=O)(=O)NC(=O)Nc3c4c(cc5c3CCC5)CCC4)cnn2C1 ; CN(C)C1CNc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1NCC1(CC1)C2 ; CC1(C)CNc2c(S(=O)(=O)NC(=O)Nc3c4c(cc5c3CCC5)CCC4)cnn2C1]

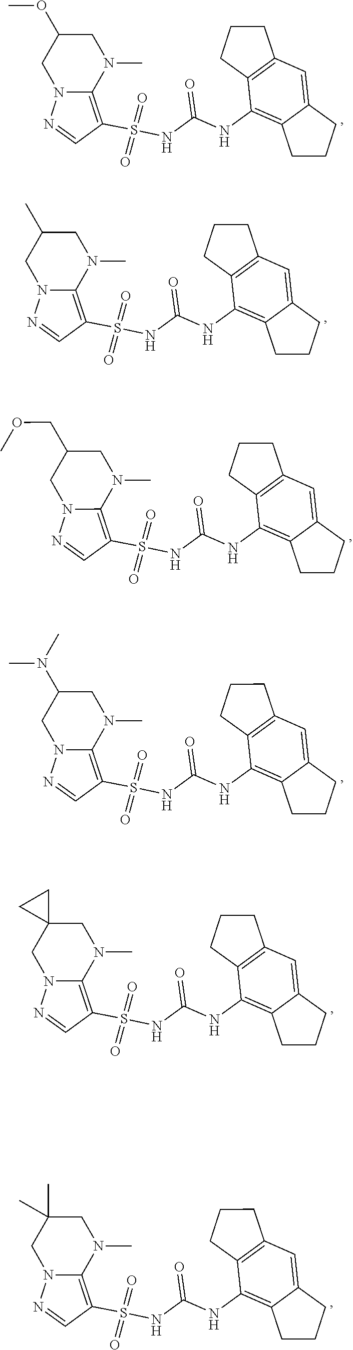 [SMILES: COC1CN(C)c2c(S(=O)(=O)NC(=O)Nc3c4c(cc5c3CCC5)CCC4)cnn2C1 ; CC1CN(C)c2c(S(=O)(=O)NC(=O)Nc3c4c(cc5c3CCC5)CCC4)cnn2C1 ; COCC1CN(C)c2c(S(=O)(=O)NC(=O)Nc3c4c(cc5c3CCC5)CCC4)cnn2C1 ; CN1CC(N(C)C)Cn2ncc(S(=O)(=O)NC(=O)Nc3c4c(cc5c3CCC5)CCC4)c21 ; CN1CC2(CC2)Cn2ncc(S(=O)(=O)NC(=O)Nc3c4c(cc5c3CCC5)CCC4)c21 ; CN1CC(C)(C)Cn2ncc(S(=O)(=O)NC(=O)Nc3c4c(cc5c3CCC5)CCC4)c21]

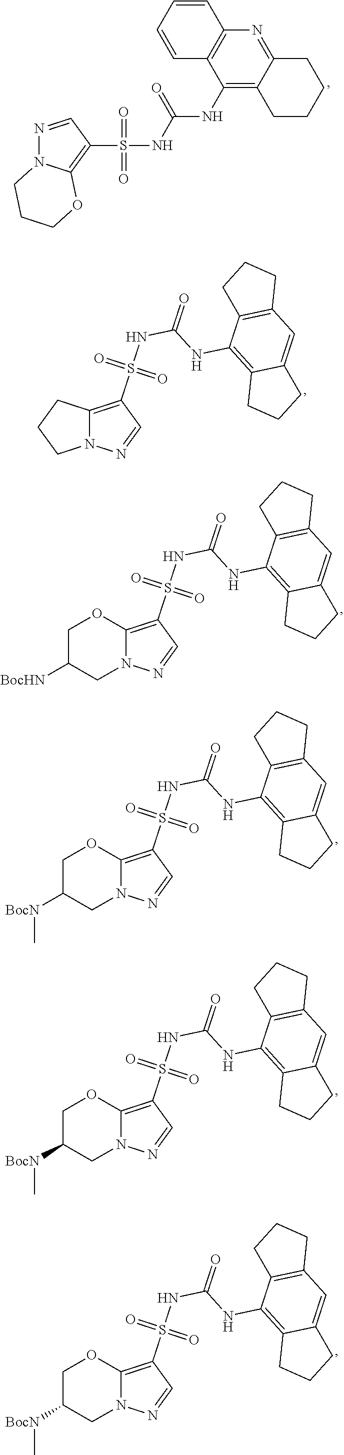 [SMILES: O=C(Nc1c2c(nc3ccccc13)CCCC2)NS(=O)(=O)c1cnn2c1OCCC2 ; O=C(Nc1c2c(cc3c1CCC3)CCC2)NS(=O)(=O)c1cnn2c1CCC2 ; CC(C)(C)OC(=O)NC1COc2c(S(=O)(=O)NC(=O)Nc3c4c(cc5c3CCC5)CCC4)cnn2C1 ; CN(C(=O)OC(C)(C)C)C1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(cc5c3CCC5)CCC4)cnn2C1 ; CN(C(=O)OC(C)(C)C)[C@@H]1COc2c(S(=O)(=O)NC(=O)Nc3c4c(cc5c3CCC5)CCC4)cnn2C1]

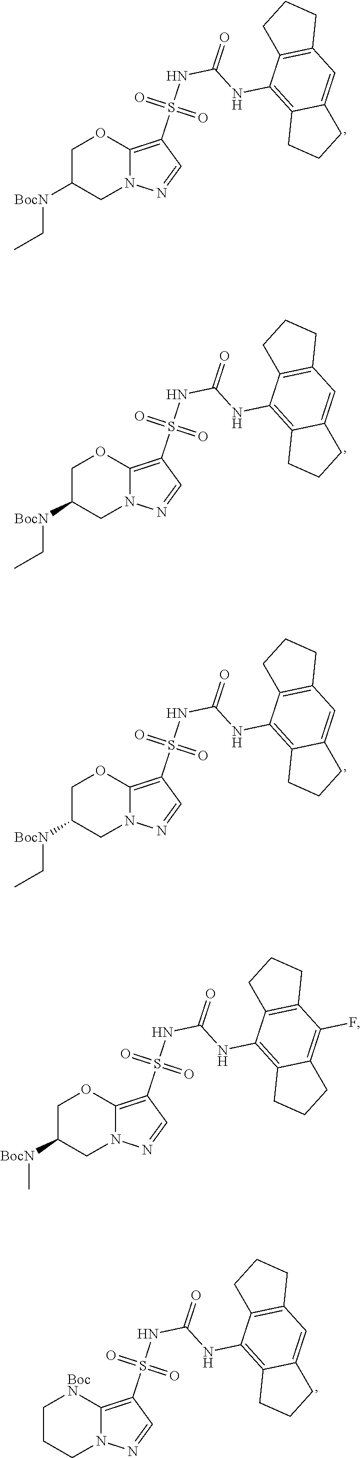 [SMILES: CCN(C(=O)OC(C)(C)C)C1COc2c(S(=O)(=O)NC(=O)Nc3c4c(cc5c3CCC5)CCC4)cnn2C1 ; CCN(C(=O)OC(C)(C)C)[C@H]1COc2c(S(=O)(=O)NC(=O)Nc3c4c(cc5c3CCC5)CCC4)cnn2C1 ; CCN(C(=O)OC(C)(C)C)[C@@H]1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(c(F)c5c3CCC5)CCC4)cnn2C1 ; CC(C)(C)OC(=O)N1CCCn2ncc(S(=O)(=O)NC(=O)Nc3c4c(cc5c3CCC5)CCC4)c21]

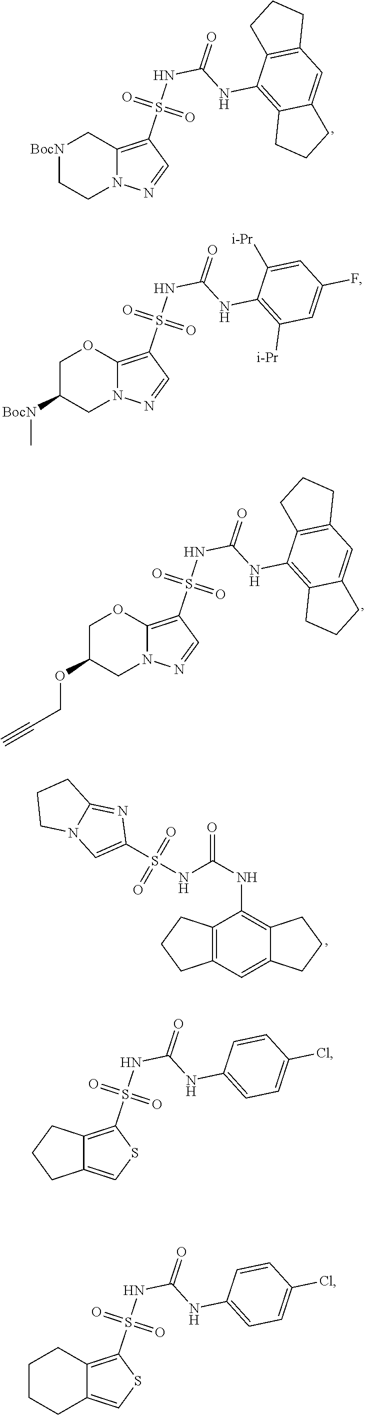 [SMILES: CC(C)(C)OC(=O)N1CCn2ncc(S(=O)(=O)NC(=O)Nc3c4c(cc5c3CCC5)CCC4)c2C1 ; CC(C)c1cc(F)cc(C(C)C)c1NC(=O)NS(=O)(=O)c1cnn2c1OC[C@H](N(C)C(=O)OC(C)(C)C)C2 ; C#CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cn2c(n1)CCC2 ; O=C(Nc1ccc(Cl)cc1)NS(=O)(=O)c1scc2c1CCC2 ; O=C(Nc1ccc(Cl)cc1)NS(=O)(=O)c1scc2c1CCCC2]

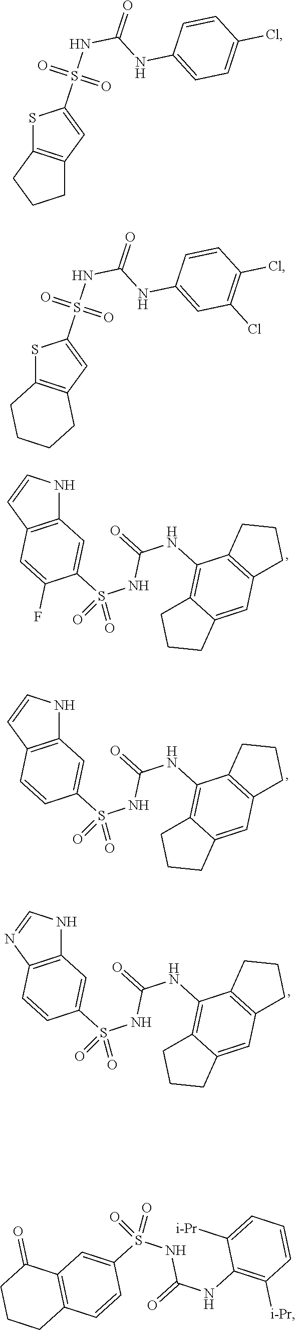

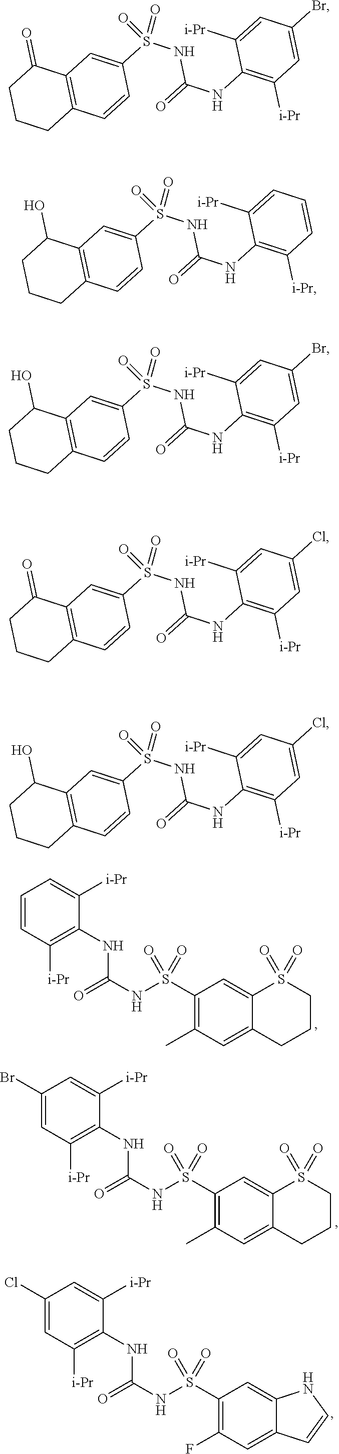

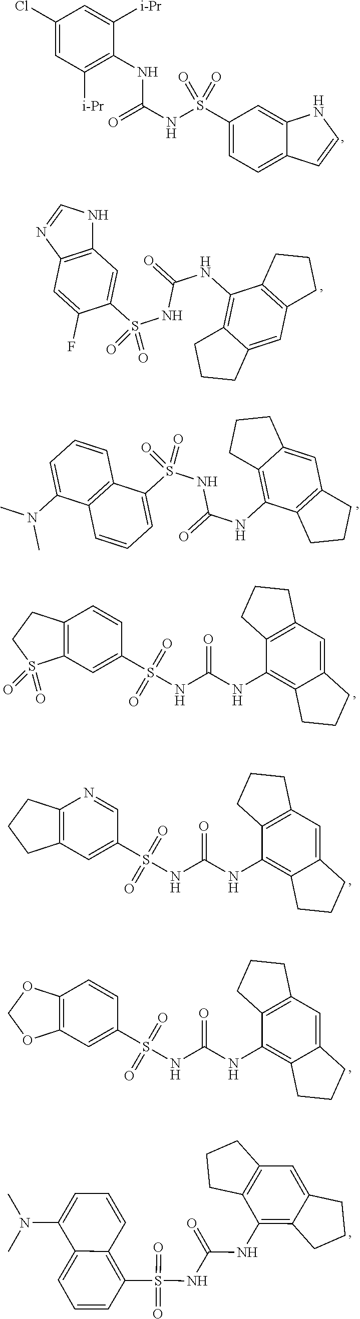

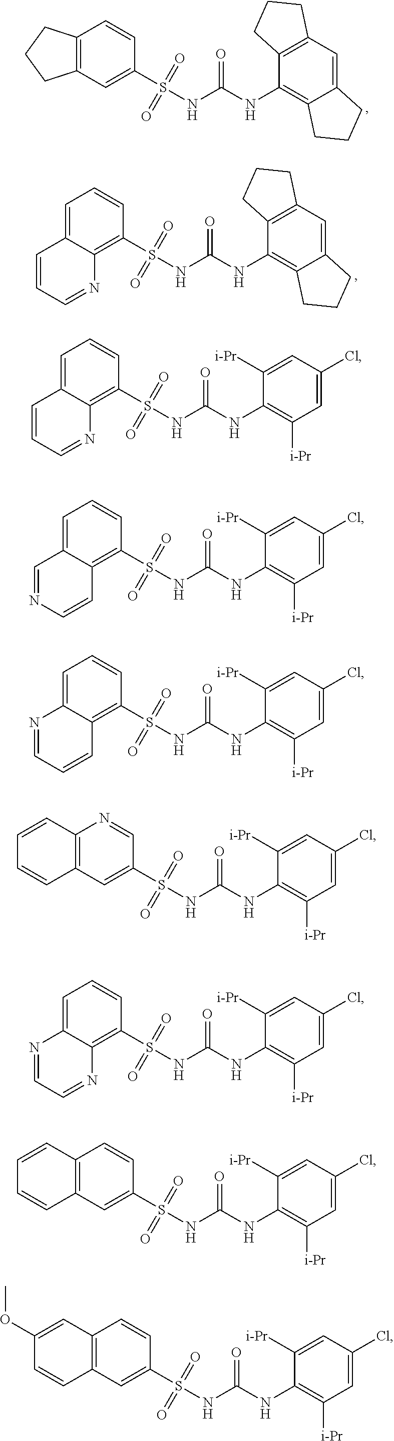

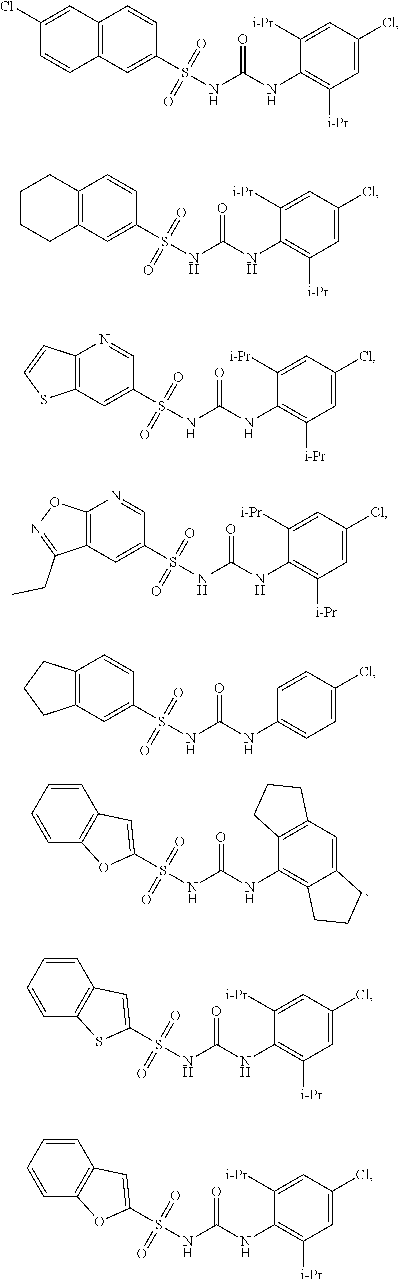

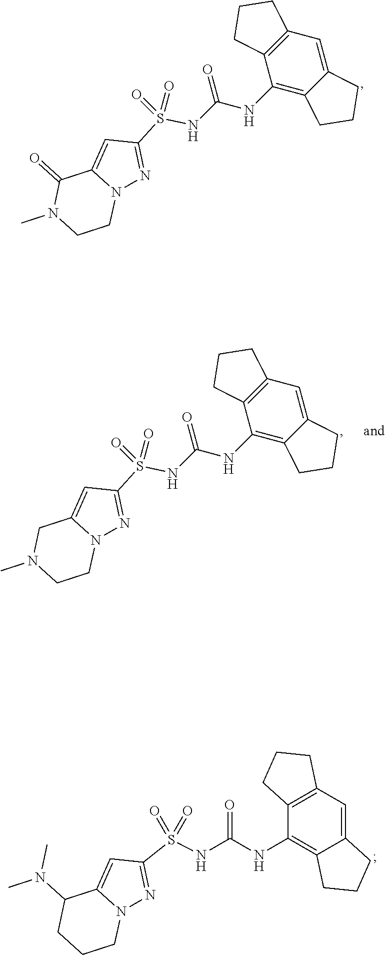

or a pharmaceutically acceptable salt thereof.

In one aspect, provided herein is a compound of Formula AA

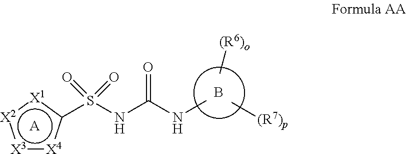

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
when X⁴ is —X⁵—X⁶, then: X¹ is N or CR¹, X² is N or CR², and X³ is N or CR³;
when X⁴ is other than —X⁵—X⁶—, then

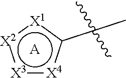

comprises at least one of CR¹, CR², CR³, and CR⁴;
when X⁴ is —X⁵—X⁶—, then

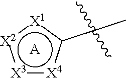

comprises at least two of CR¹, CR², CR³, CR⁵, and CR⁶;
from two to four of R¹, R², R³, and R⁴ are present or from two to five of R¹, R², R³, R⁵, and R⁶ are present; and
wherein at least two of the two to four R¹, R², R³, and R⁴ or at least of the two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰;

R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, —OC(O)NHC₆₋₁₀ aryl optionally substituted with one or more R²¹, and CONR⁸R⁹;
or at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;
R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
or at least one pair of R²¹ on adjacent atoms, taken together with the atoms connecting them, independently forms a C₄-C₁₂ cycloalkyl ring or a 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;
wherein any of R¹, R², R³, R⁴, R⁵, and R⁶ that are not taken together with the atoms connecting them to form a ring, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;
R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₁-C₆ alkynyl optionally substituted with one or more R²³;
R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
B is a 5-10-membered heteroaryl or C₆-C₁₀ aryl ring;
o=1 or 2;
p=0, 1, 2, or 3;
R⁶ and R⁷ are each independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(CO₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵,
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(CO₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
provided that when B is 5-10-membered heteroaryl with from 2-3 ring nitrogen atoms, at least one R⁶ is bonded to B at a position ortho to the —HNC(═O)NHS(O)₂— moiety of Formula AA;
when B is 2-pyridyl, pyrimidin-6-yl, or pyrimidin-4-yl, B is not substituted with a cyano group at a position ortho to the —HNC(═O)NHS(O)₂— moiety of Formula AA;
and
with the proviso that the compound of Formula AA is not a compound selected from the group consisting of:

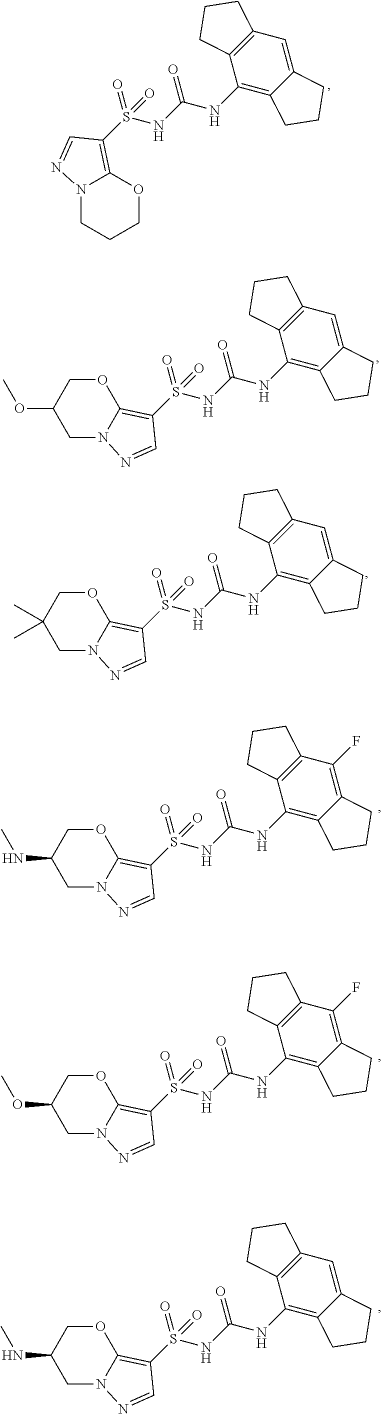 [SMILES: O=C(Nc1c2c(cc3c1CCC3)CCC2)NS(=O)(=O)c1cnn2c1OCCC2 ; COC1COc2c(S(=O)(=O)NC(=O)Nc3c4c(cc5c3CCC5)CCC4)cnn2C1 ; CC1(C)COc2c(S(=O)(=O)NC(=O)Nc3c4c(cc5c3CCC5)CCC4)cnn2C1 ; CN[C@H]1COc2c(S(=O)(=O)NC(=O)Nc3c4c(c(F)c5c3CCC5)CCC4)cnn2C1 ; CO[C@H]1COc2c(S(=O)(=O)NC(=O)Nc3c4c(c(F)c5c3CCC5)CCC4)cnn2C1 ; CN[C@H]1COc2c(S(=O)(=O)NC(=O)Nc3c4c(cc5c3CCC5)CCC4)cnn2C1]

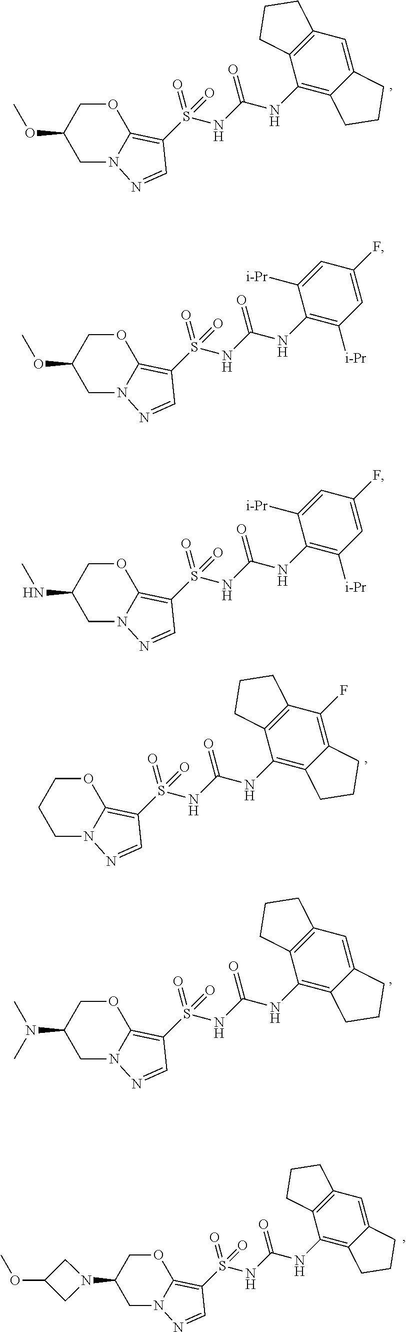 [SMILES: CO[C@H]1COc2c(S(=O)(=O)NC(=O)Nc3c4c(cc5c3CCC5)CCC4)cnn2C1 ; CO[C@H]1COc2c(S(=O)(=O)NC(=O)Nc3c(C(C)C)cc(F)cc3C(C)C)cnn2C1 ; CN[C@H]1COc2c(S(=O)(=O)NC(=O)Nc3c(C(C)C)cc(F)cc3C(C)C)cnn2C1 ; O=C(Nc1c2c(c(F)c3c1CCC3)CCC2)NS(=O)(=O)c1cnn2c1OCCC2 ; CN(C)[C@H]1COc2c(S(=O)(=O)NC(=O)Nc3c4c(cc5c3CCC5)CCC4)cnn2C1 ; COC1CN([C@H]2COc3c(S(=O)(=O)NC(=O)Nc4c5c(cc6c4CCC6)CCC5)cnn3C2)C1]

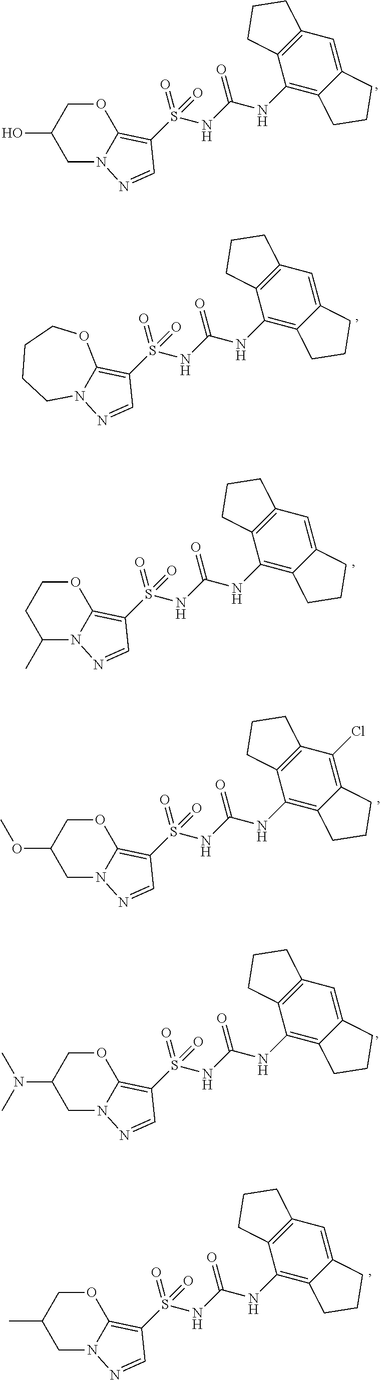 [SMILES: O=C(Nc1c2c(cc3c1CCC3)CCC2)NS(=O)(=O)c1cnn2c1OCC(O)C2 ; O=C(Nc1c2c(cc3c1CCC3)CCC2)NS(=O)(=O)c1cnn2c1OCCCC2 ; CC1CCOc2c(S(=O)(=O)NC(=O)Nc3c4c(cc5c3CCC5)CCC4)cnn21 ; COC1COc2c(S(=O)(=O)NC(=O)Nc3c4c(c(Cl)c5c3CCC5)CCC4)cnn2C1 ; CN(C)C1COc2c(S(=O)(=O)NC(=O)Nc3c4c(cc5c3CCC5)CCC4)cnn2C1 ; CC1COc2c(S(=O)(=O)NC(=O)Nc3c4c(cc5c3CCC5)CCC4)cnn2C1]

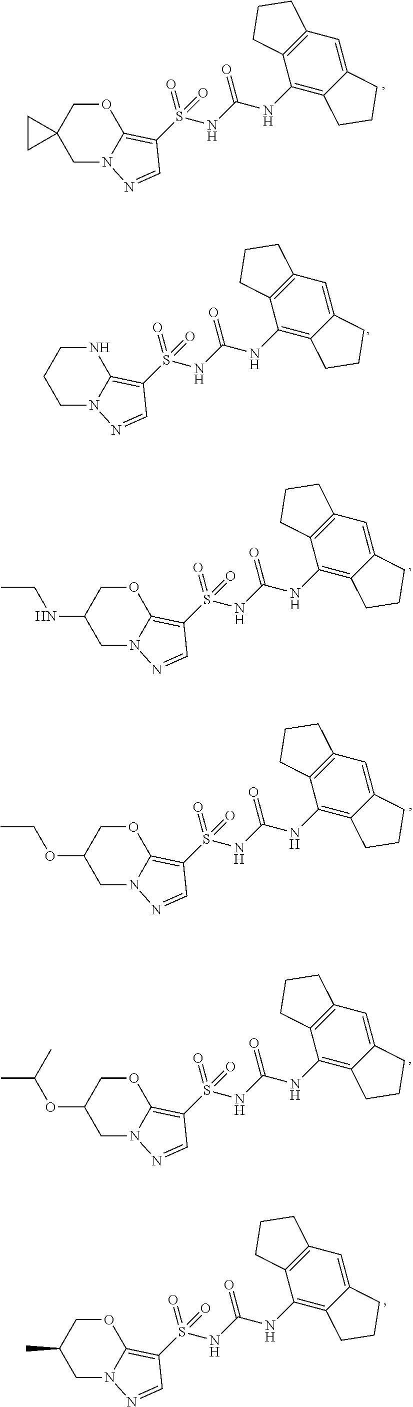 [SMILES: O=C(Nc1c2c(cc3c1CCC3)CCC2)NS(=O)(=O)c1cnn2c1OCC1(CC1)C2 ; O=C(Nc1c2c(cc3c1CCC3)CCC2)NS(=O)(=O)c1cnn2c1NCCC2 ; CCNC1COc2c(S(=O)(=O)NC(=O)Nc3c4c(cc5c3CCC5)CCC4)cnn2C1 ; CCOC1COc2c(S(=O)(=O)NC(=O)Nc3c4c(cc5c3CCC5)CCC4)cnn2C1 ; CC(C)OC1COc2c(S(=O)(=O)NC(=O)Nc3c4c(cc5c3CCC5)CCC4)cnn2C1 ; C[C@H]1COc2c(S(=O)(=O)NC(=O)Nc3c4c(cc5c3CCC5)CCC4)cnn2C1]

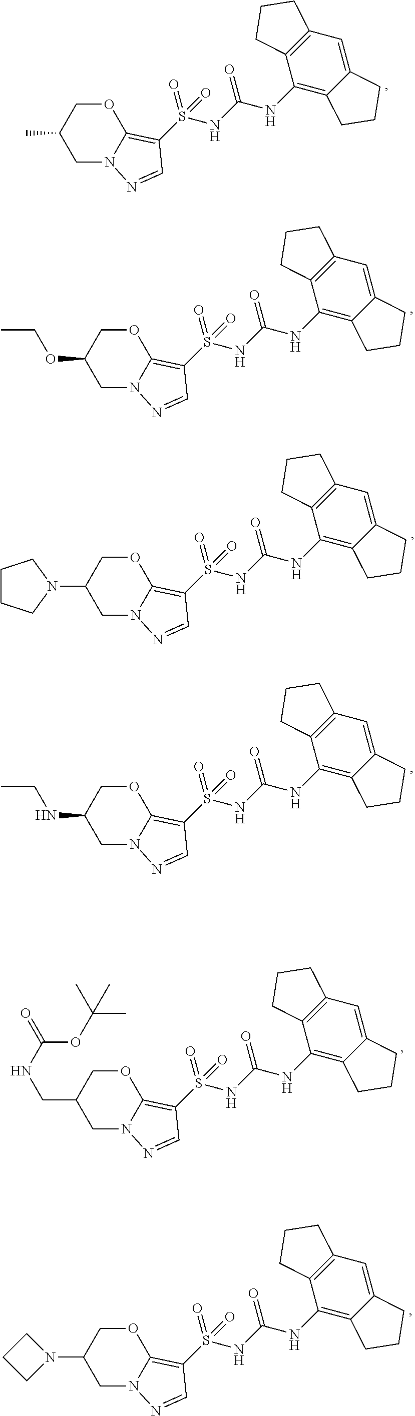 [SMILES: C[C@@H]1COc2c(S(=O)(=O)NC(=O)Nc3c4c(cc5c3CCC5)CCC4)cnn2C1 ; CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCCC1)C2 ; CCN[C@H]1COc2c(S(=O)(=O)NC(=O)Nc3c4c(cc5c3CCC5)CCC4)cnn2C1 ; CC(C)(C)OC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCC1)C2]

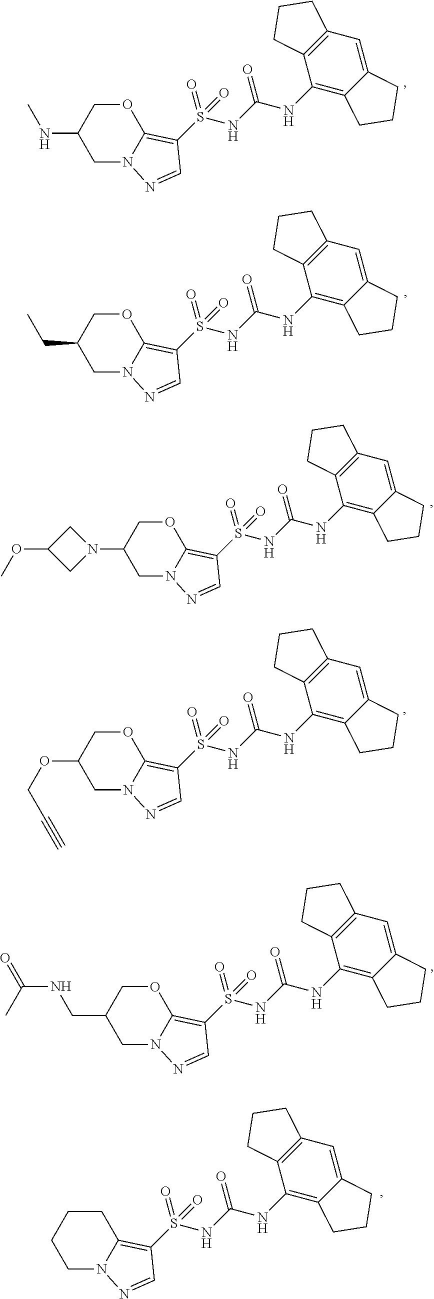 [SMILES: CNC1COc2c(S(=O)(=O)NC(=O)Nc3c4c(cc5c3CCC5)CCC4)cnn2C1 ; CC[C@H]1COc2c(S(=O)(=O)NC(=O)Nc3c4c(cc5c3CCC5)CCC4)cnn2C1 ; COC1CN(C2COc3c(S(=O)(=O)NC(=O)Nc4c5c(cc6c4CCC6)CCC5)cnn3C2)C1 ; C#CCOC1COc2c(S(=O)(=O)NC(=O)Nc3c4c(cc5c3CCC5)CCC4)cnn2C1 ; CC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1CCCC2]

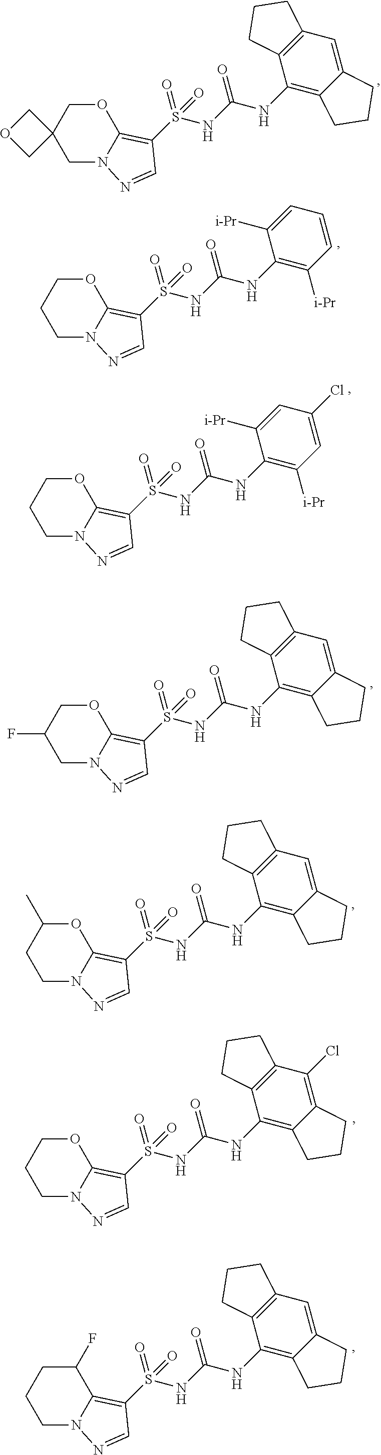

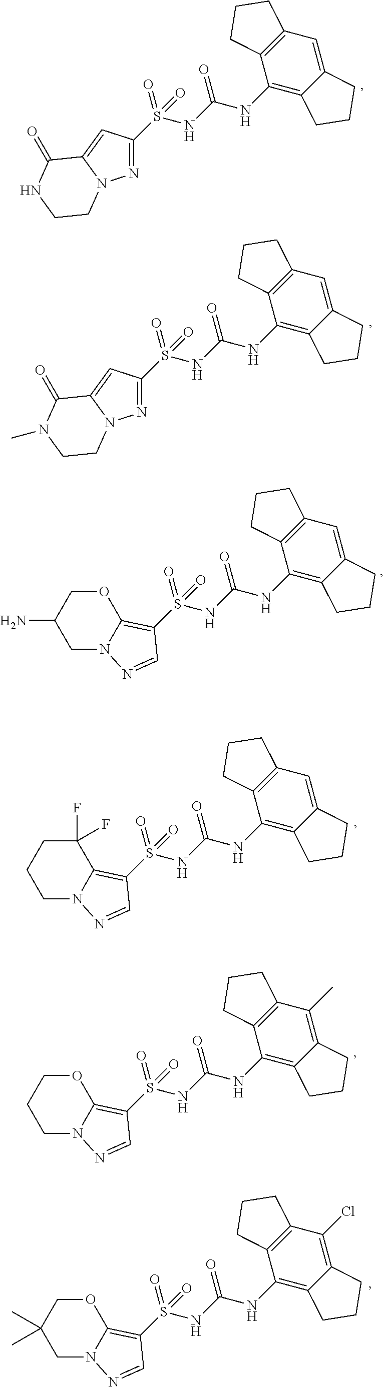 [SMILES: O=C(Nc1c2c(cc3c1CCC3)CCC2)NS(=O)(=O)c1cc2n(n1)CCNC2=O ; CN1CCn2nc(S(=O)(=O)NC(=O)Nc3c4c(cc5c3CCC5)CCC4)cc2C1=O ; NC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1C(F)(F)CCC2 ; Cc1c2c(c(NC(=O)NS(=O)(=O)c3cnn4c3OCCC4)c3c1CCC3)CCC2 ; CC1(C)COc2c(S(=O)(=O)NC(=O)Nc3c4c(c(Cl)c5c3CCC5)CCC4)cnn2C1]

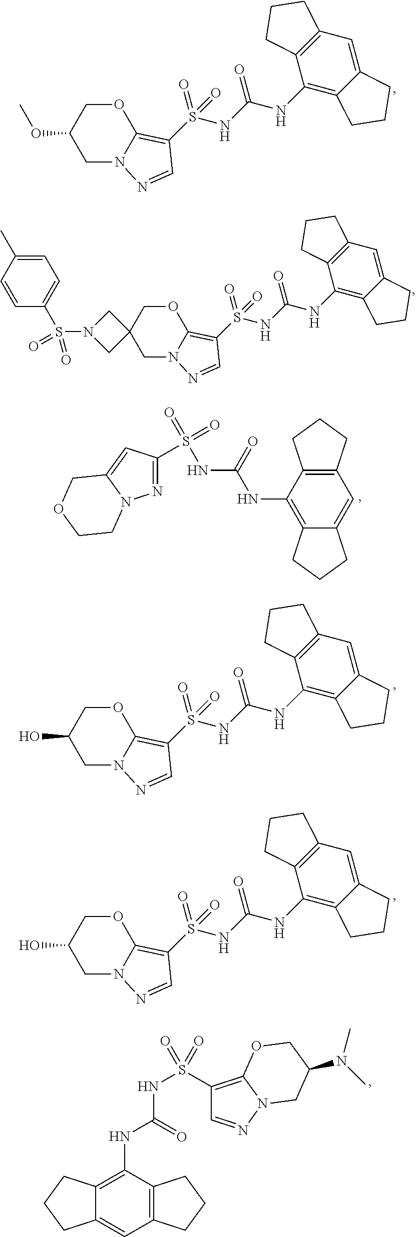

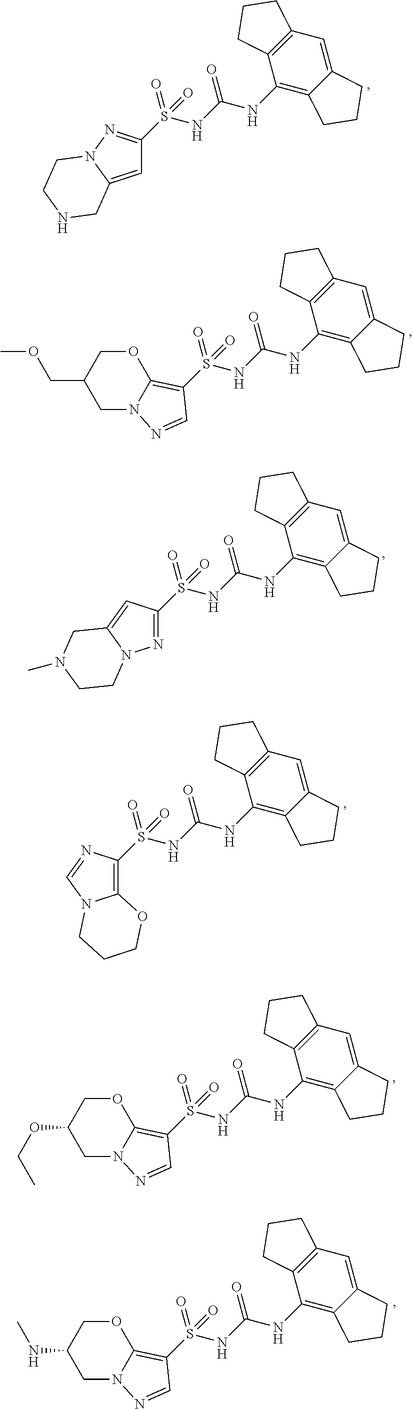 [SMILES: O=C(Nc1c2c(cc3c1CCC3)CCC2)NS(=O)(=O)c1cc2n(n1)CCNC2 ; COCC1COc2c(S(=O)(=O)NC(=O)Nc3c4c(cc5c3CCC5)CCC4)cnn2C1 ; CN1CCn2nc(S(=O)(=O)NC(=O)Nc3c4c(cc5c3CCC5)CCC4)cc2C1 ; O=C(Nc1c2c(cc3c1CCC3)CCC2)NS(=O)(=O)c1ncn2c1OCCC2 ; CCO[C@@H]1COc2c(S(=O)(=O)NC(=O)Nc3c4c(cc5c3CCC5)CCC4)cnn2C1 ; CN[C@@H]1COc2c(S(=O)(=O)NC(=O)Nc3c4c(cc5c3CCC5)CCC4)cnn2C1]

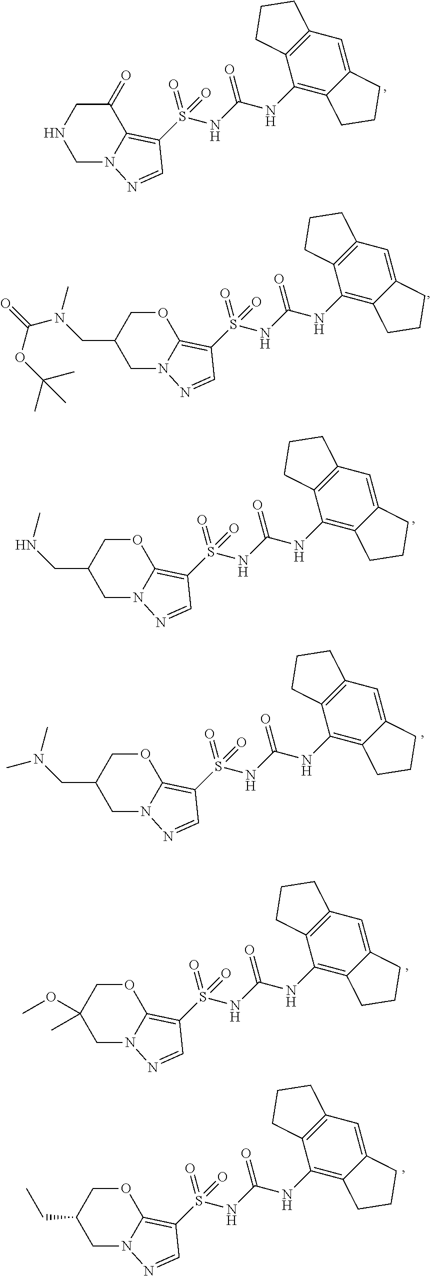 [SMILES: O=C(Nc1c2c(cc3c1CCC3)CCC2)NS(=O)(=O)c1cnn2c1C(=O)CNC2 ; CN(CC1COc2c(S(=O)(=O)NC(=O)Nc3c4c(cc5c3CCC5)CCC4)cnn2C1)C(=O)OC(C)(C)C ; CNCC1COc2c(S(=O)(=O)NC(=O)Nc3c4c(cc5c3CCC5)CCC4)cnn2C1 ; CN(C)CC1COc2c(S(=O)(=O)NC(=O)Nc3c4c(cc5c3CCC5)CCC4)cnn2C1 ; COC1(C)COc2c(S(=O)(=O)NC(=O)Nc3c4c(cc5c3CCC5)CCC4)cnn2C1 ; CC[C@@H]1COc2c(S(=O)(=O)NC(=O)Nc3c4c(cc5c3CCC5)CCC4)cnn2C1]

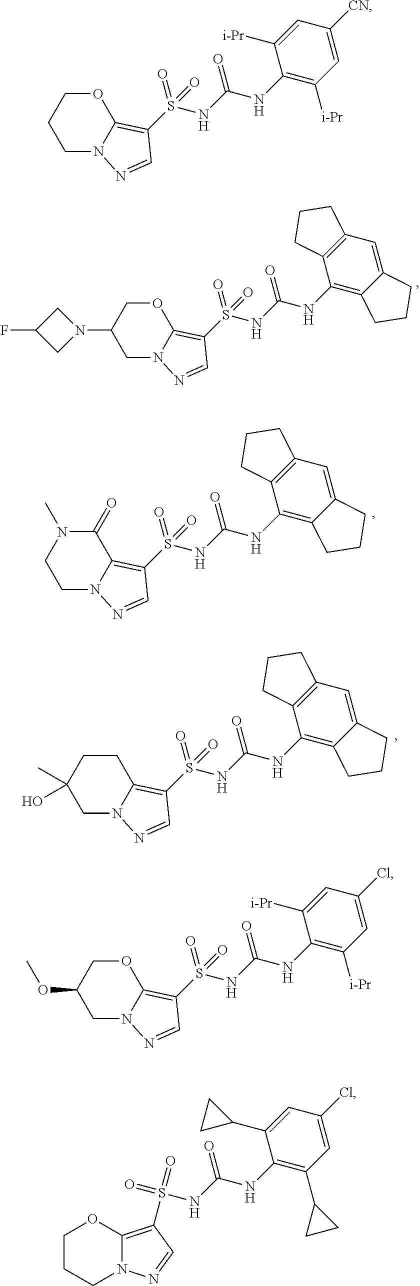 [SMILES: CC(C)c1cc(C#N)cc(C(C)C)c1NC(=O)NS(=O)(=O)c1cnn2c1OCCC2 ; O=C(Nc1c2c(cc3c1CCC3)CCC2)NS(=O)(=O)c1cnn2c1OCC(N1CC(F)C1)C2 ; CN1CCn2ncc(S(=O)(=O)NC(=O)Nc3c4c(cc5c3CCC5)CCC4)c2C1=O ; CC1(O)CCc2c(S(=O)(=O)NC(=O)Nc3c4c(cc5c3CCC5)CCC4)cnn2C1 ; CO[C@H]1COc2c(S(=O)(=O)NC(=O)Nc3c(C(C)C)cc(Cl)cc3C(C)C)cnn2C1 ; O=C(Nc1c(C2CC2)cc(Cl)cc1C1CC1)NS(=O)(=O)c1cnn2c1OCCC2]

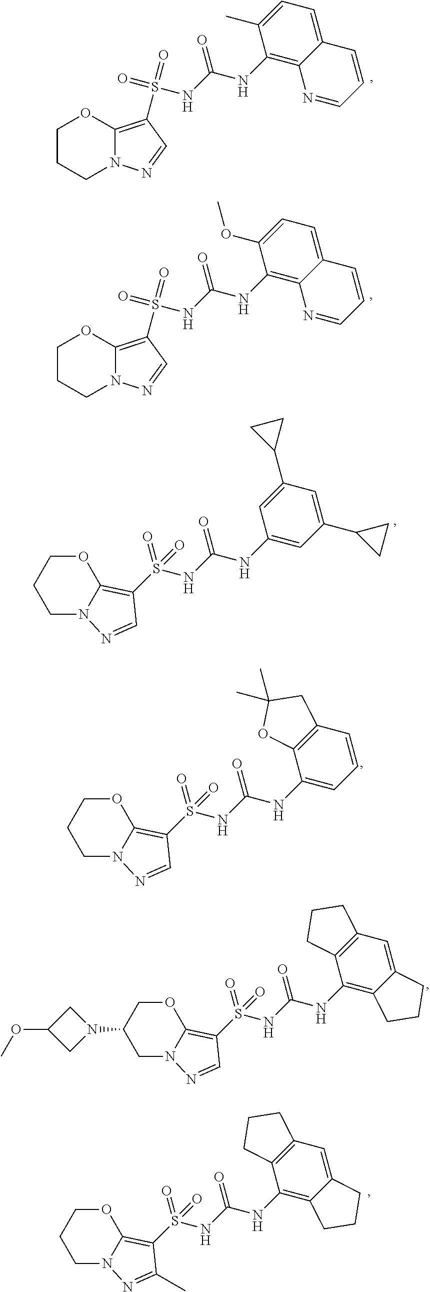 [SMILES: Cc1ccc2cccnc2c1NC(=O)NS(=O)(=O)c1cnn2c1OCCC2 ; COc1ccc2cccnc2c1NC(=O)NS(=O)(=O)c1cnn2c1OCCC2 ; O=C(Nc1cc(C2CC2)cc(C2CC2)c1)NS(=O)(=O)c1cnn2c1OCCC2 ; CC1(C)Cc2cccc(NC(=O)NS(=O)(=O)c3cnn4c3OCCC4)c2O1 ; COC1CN([C@@H]2COc3c(S(=O)(=O)NC(=O)Nc4c5c(cc6c4CCC6)CCC5)cnn3C2)C1 ; Cc1nn2c(c1S(=O)(=O)NC(=O)Nc1c3c(cc4c1CCC4)CCC3)OCCC2]

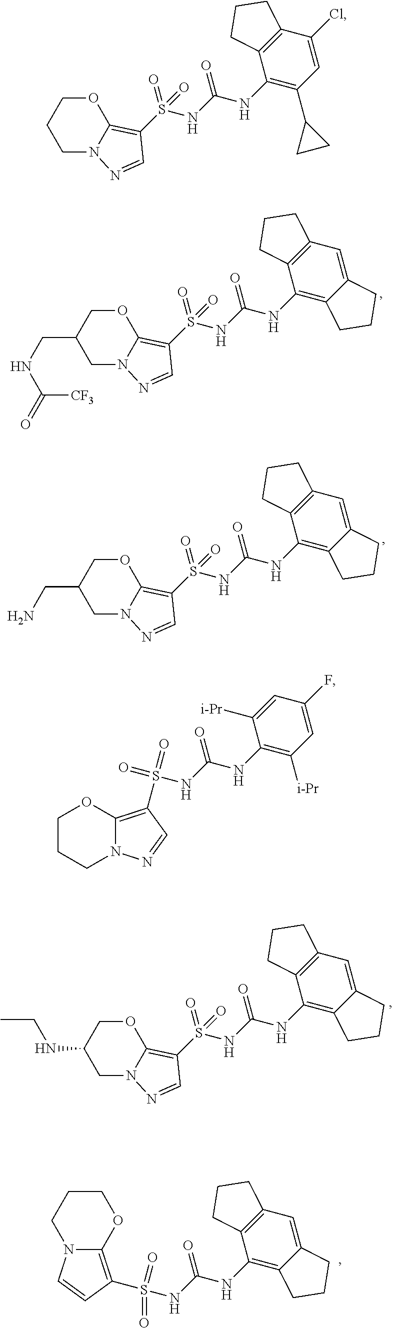 [SMILES: O=C(Nc1c(C2CC2)cc(Cl)c2c1CCC2)NS(=O)(=O)c1cnn2c1OCCC2 ; O=C(Nc1c2c(cc3c1CCC3)CCC2)NS(=O)(=O)c1cnn2c1OCC(CNC(=O)C(F)(F)F)C2 ; NCC1COc2c(S(=O)(=O)NC(=O)Nc3c4c(cc5c3CCC5)CCC4)cnn2C1 ; CC(C)c1cc(F)cc(C(C)C)c1NC(=O)NS(=O)(=O)c1cnn2c1OCCC2 ; CCN[C@@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1ccn2c1OCCC2]

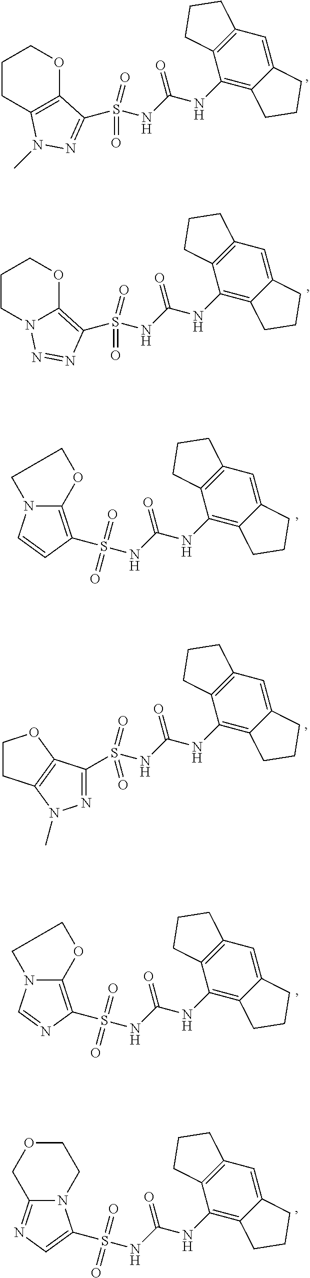 [SMILES: Cn1nc(S(=O)(=O)NC(=O)Nc2c3c(cc4c2CCC4)CCC3)c2c1CCCO2 ; O=C(Nc1c2c(cc3c1CCC3)CCC2)NS(=O)(=O)c1nnn2c1OCCC2 ; O=C(Nc1c2c(cc3c1CCC3)CCC2)NS(=O)(=O)c1ccn2c1OCC2 ; Cn1nc(S(=O)(=O)NC(=O)Nc2c3c(cc4c2CCC4)CCC3)c2c1CCO2 ; O=C(Nc1c2c(cc3c1CCC3)CCC2)NS(=O)(=O)c1ncn2c1OCC2 ; O=C(Nc1c2c(cc3c1CCC3)CCC2)NS(=O)(=O)c1cnc2n1CCOC2]

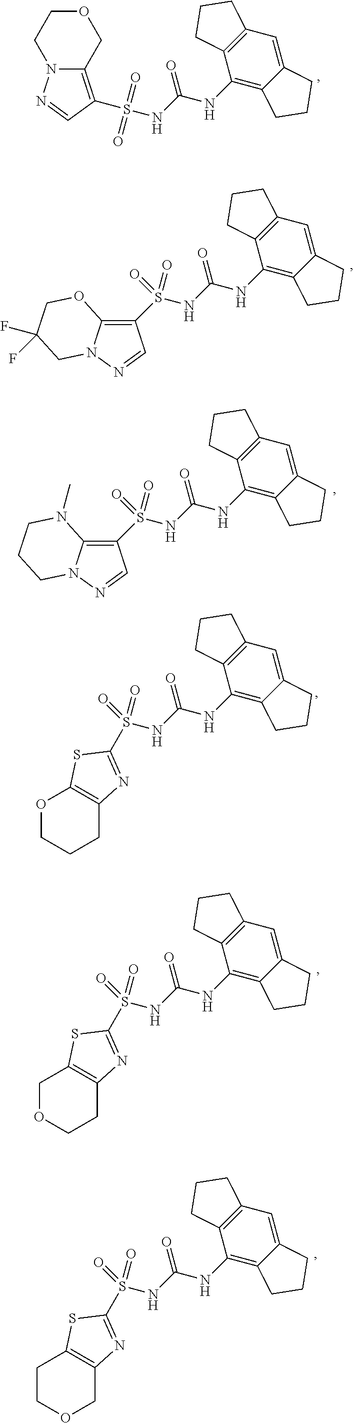 [SMILES: O=C(Nc1c2c(cc3c1CCC3)CCC2)NS(=O)(=O)c1cnn2c1COCC2 ; O=C(Nc1c2c(cc3c1CCC3)CCC2)NS(=O)(=O)c1cnn2c1OCC(F)(F)C2 ; CN1CCCn2ncc(S(=O)(=O)NC(=O)Nc3c4c(cc5c3CCC5)CCC4)c21 ; O=C(Nc1c2c(cc3c1CCC3)CCC2)NS(=O)(=O)c1nc2c(s1)OCCC2 ; O=C(Nc1c2c(cc3c1CCC3)CCC2)NS(=O)(=O)c1nc2c(s1)COCC2 ; O=C(Nc1c2c(cc3c1CCC3)CCC2)NS(=O)(=O)c1nc2c(s1)CCOC2]

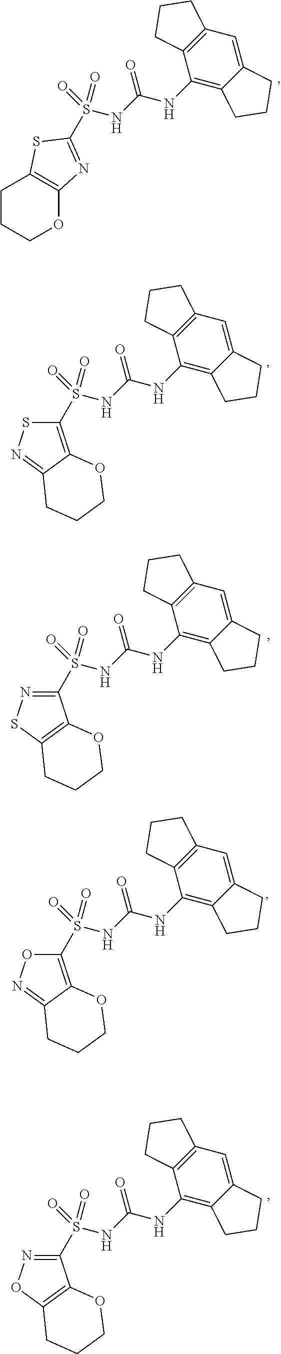 [SMILES: O=C(Nc1c2c(cc3c1CCC3)CCC2)NS(=O)(=O)c1nc2c(s1)CCCO2 ; O=C(Nc1c2c(cc3c1CCC3)CCC2)NS(=O)(=O)c1snc2c1OCCC2 ; O=C(Nc1c2c(cc3c1CCC3)CCC2)NS(=O)(=O)c1nsc2c1OCCC2 ; O=C(Nc1c2c(cc3c1CCC3)CCC2)NS(=O)(=O)c1onc2c1OCCC2 ; O=C(Nc1c2c(cc3c1CCC3)CCC2)NS(=O)(=O)c1noc2c1OCCC2]

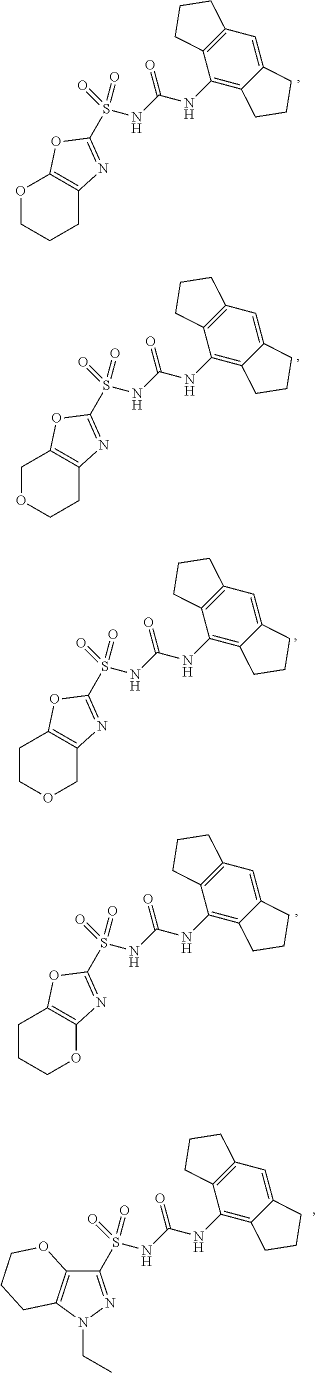 [SMILES: O=C(Nc1c2c(cc3c1CCC3)CCC2)NS(=O)(=O)c1nc2c(o1)OCCC2 ; O=C(Nc1c2c(cc3c1CCC3)CCC2)NS(=O)(=O)c1nc2c(o1)COCC2 ; O=C(Nc1c2c(cc3c1CCC3)CCC2)NS(=O)(=O)c1nc2c(o1)CCOC2 ; O=C(Nc1c2c(cc3c1CCC3)CCC2)NS(=O)(=O)c1nc2c(o1)CCCO2 ; CCn1nc(S(=O)(=O)NC(=O)Nc2c3c(cc4c2CCC4)CCC3)c2c1CCCO2]

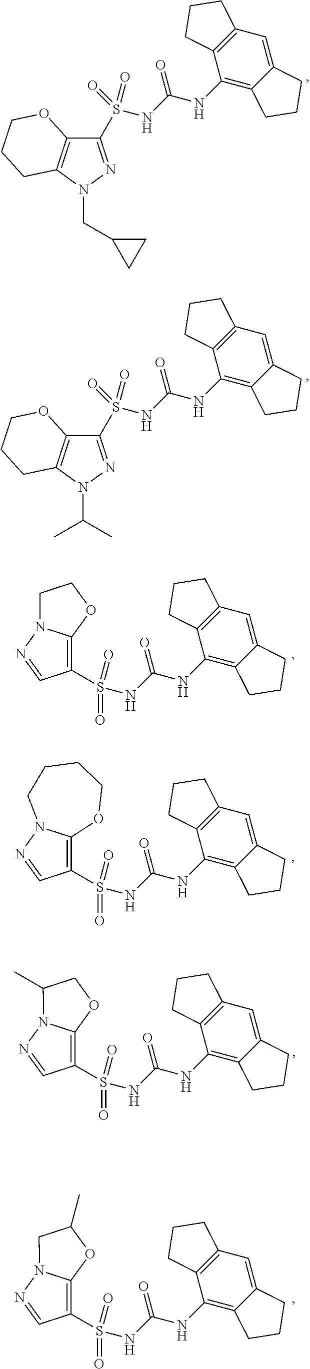 [SMILES: O=C(Nc1c2c(cc3c1CCC3)CCC2)NS(=O)(=O)c1nn(CC2CC2)c2c1OCCC2 ; CC(C)n1nc(S(=O)(=O)NC(=O)Nc2c3c(cc4c2CCC4)CCC3)c2c1CCCO2 ; O=C(Nc1c2c(cc3c1CCC3)CCC2)NS(=O)(=O)c1cnn2c1OCC2 ; O=C(Nc1c2c(cc3c1CCC3)CCC2)NS(=O)(=O)c1cnn2c1OCCCC2 ; CC1COc2c(S(=O)(=O)NC(=O)Nc3c4c(cc5c3CCC5)CCC4)cnn21 ; CC1Cn2ncc(S(=O)(=O)NC(=O)Nc3c4c(cc5c3CCC5)CCC4)c2O1]

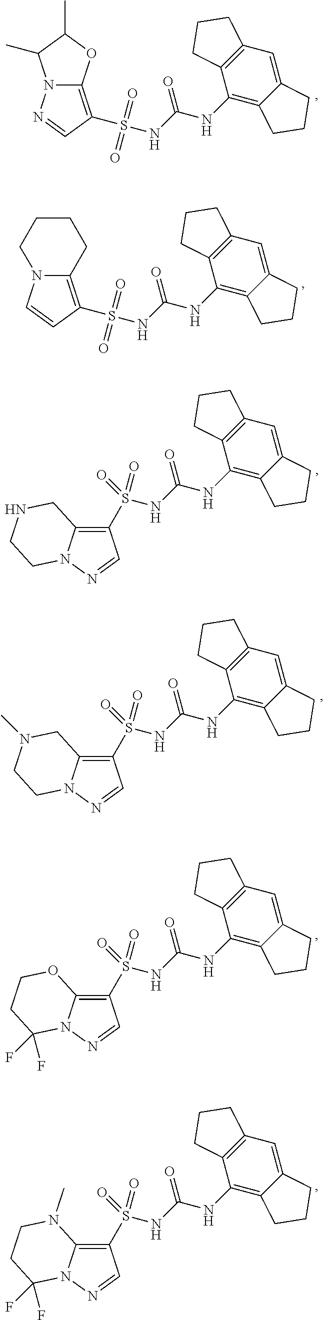 [SMILES: CC1Oc2c(S(=O)(=O)NC(=O)Nc3c4c(cc5c3CCC5)CCC4)cnn2C1C ; O=C(Nc1c2c(cc3c1CCC3)CCC2)NS(=O)(=O)c1ccn2c1CCCC2 ; O=C(Nc1c2c(cc3c1CCC3)CCC2)NS(=O)(=O)c1cnn2c1CNCC2 ; CN1CCn2ncc(S(=O)(=O)NC(=O)Nc3c4c(cc5c3CCC5)CCC4)c2C1 ; O=C(Nc1c2c(cc3c1CCC3)CCC2)NS(=O)(=O)c1cnn2c1OCCC2(F)F ; CN1CCC(F)(F)n2ncc(S(=O)(=O)NC(=O)Nc3c4c(cc5c3CCC5)CCC4)c21]

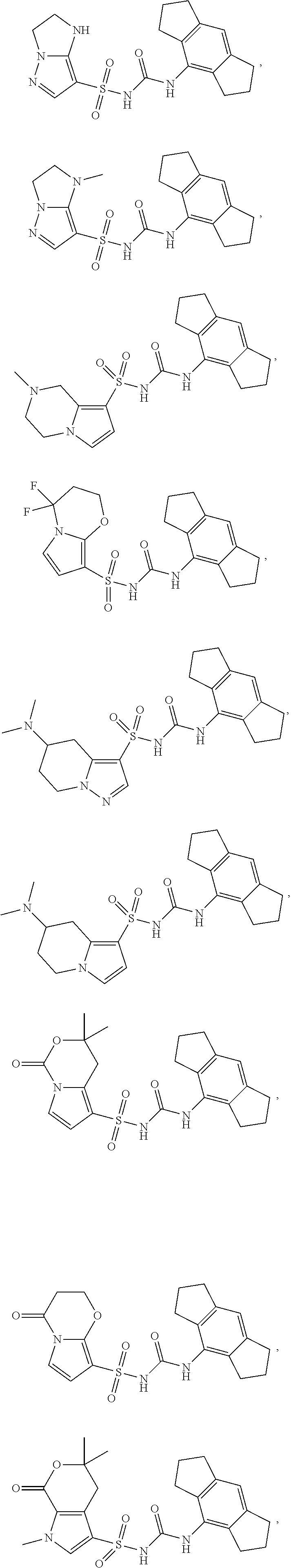

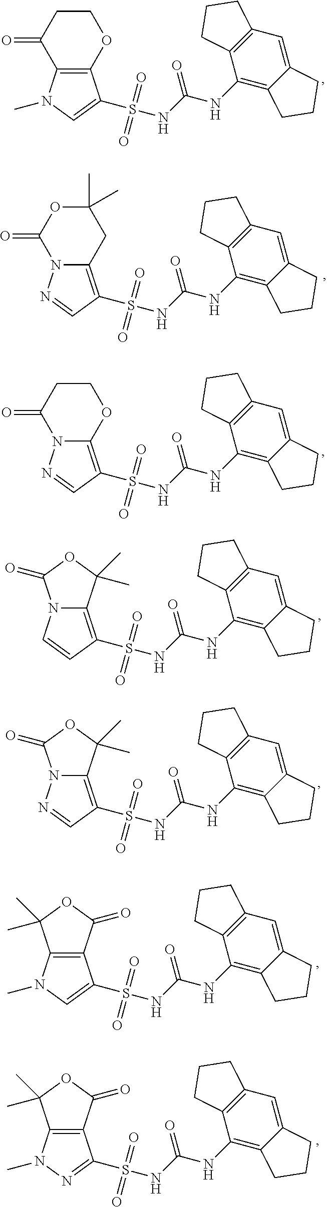

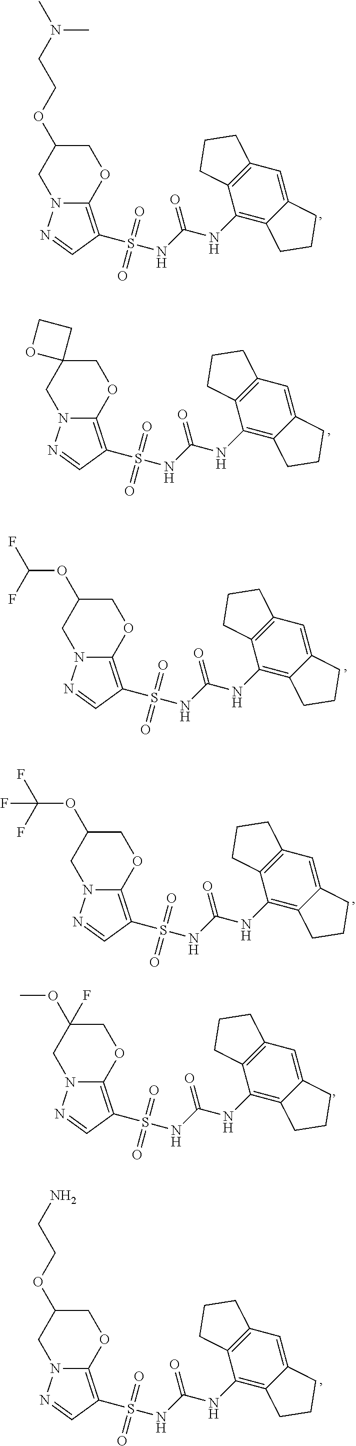 [SMILES: CN(C)CCOC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC1(CCO1)C2 ; O=C(Nc1c2c(cc3c1CCC3)CCC2)NS(=O)(=O)c1cnn2c1OCC(OC(F)F)C2 ; O=C(Nc1c2c(cc3c1CCC3)CCC2)NS(=O)(=O)c1cnn2c1OCC(OC(F)(F)F)C2 ; COC1(F)COc2c(S(=O)(=O)NC(=O)Nc3c4c(cc5c3CCC5)CCC4)cnn2C1 ; NCCOC1COc2c(S(=O)(=O)NC(=O)Nc3c4c(cc5c3CCC5)CCC4)cnn2C1]

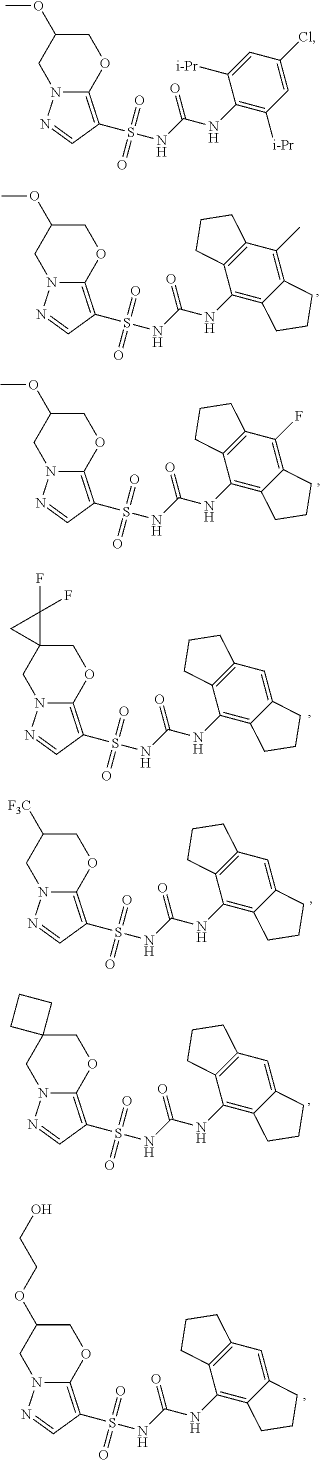

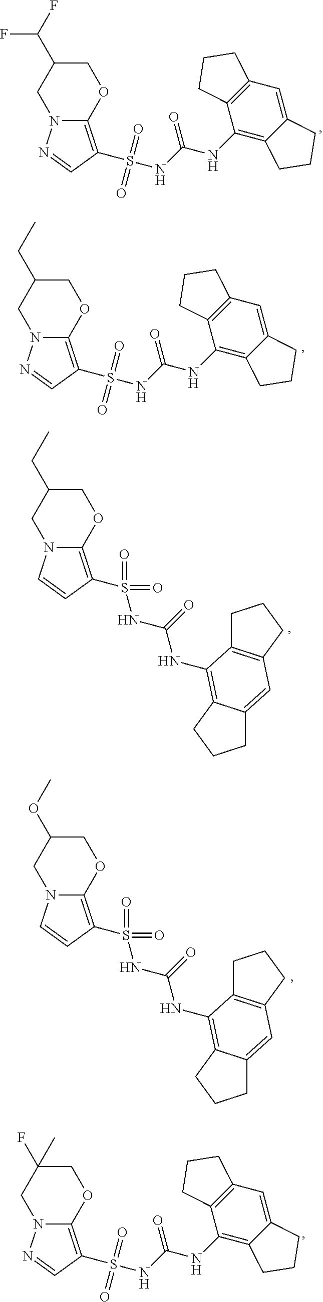

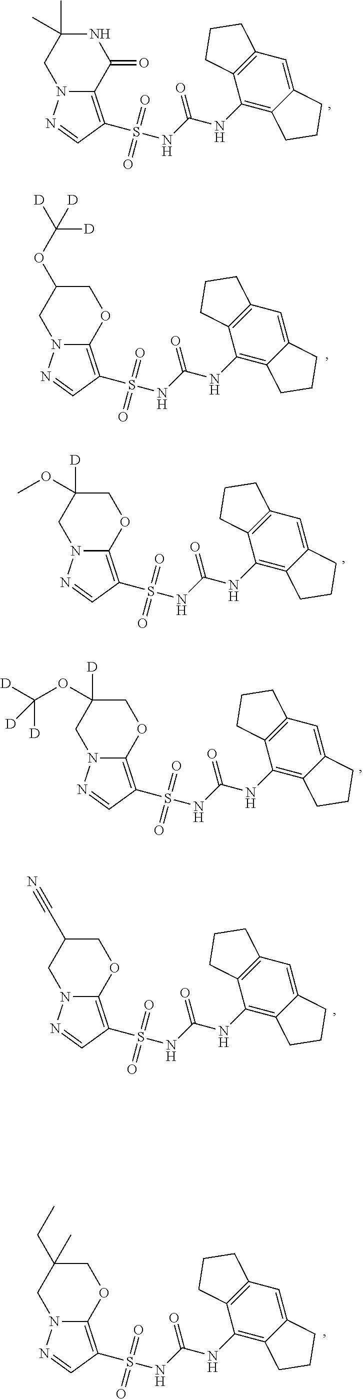 [SMILES: CC1(C)Cn2ncc(S(=O)(=O)NC(=O)Nc3c4c(cc5c3CCC5)CCC4)c2C(=O)N1 ; [2H]C([2H])([2H])OC1COc2c(S(=O)(=O)NC(=O)Nc3c4c(cc5c3CCC5)CCC4)cnn2C1 ; [2H]C1(OC)COc2c(S(=O)(=O)NC(=O)Nc3c4c(cc5c3CCC5)CCC4)cnn2C1 ; [2H]C([2H])([2H])OC1([2H])COc2c(S(=O)(=O)NC(=O)Nc3c4c(cc5c3CCC5)CCC4)cnn2C1 ; N#CC1COc2c(S(=O)(=O)NC(=O)Nc3c4c(cc5c3CCC5)CCC4)cnn2C1 ; CCC1(C)COc2c(S(=O)(=O)NC(=O)Nc3c4c(cc5c3CCC5)CCC4)cnn2C1]

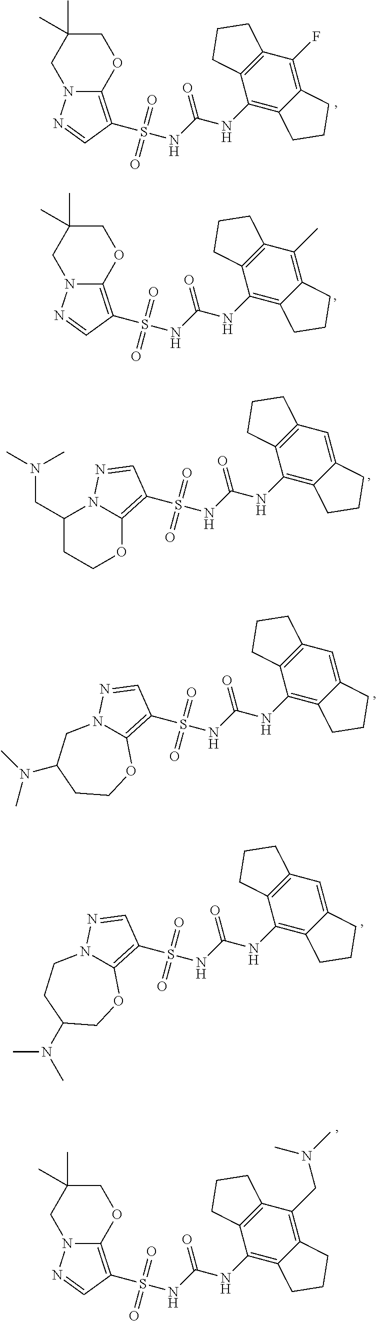

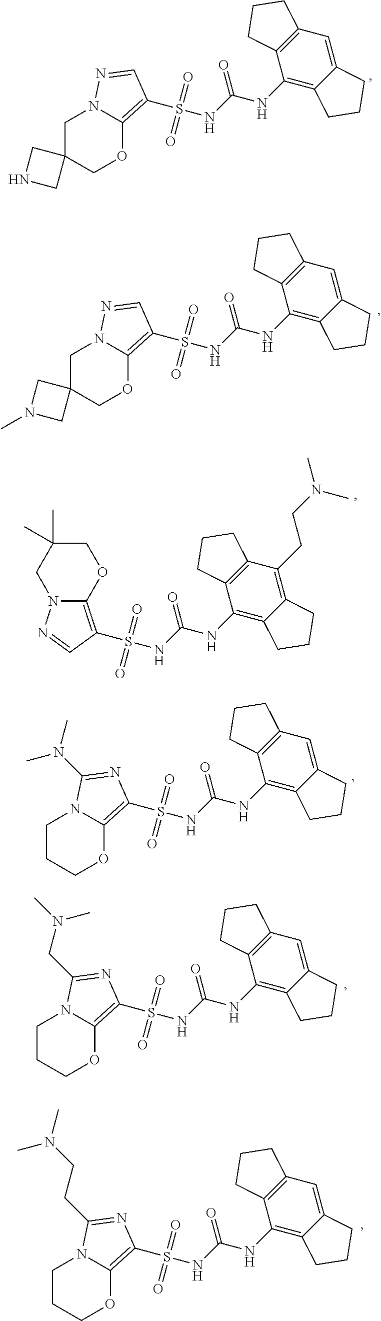

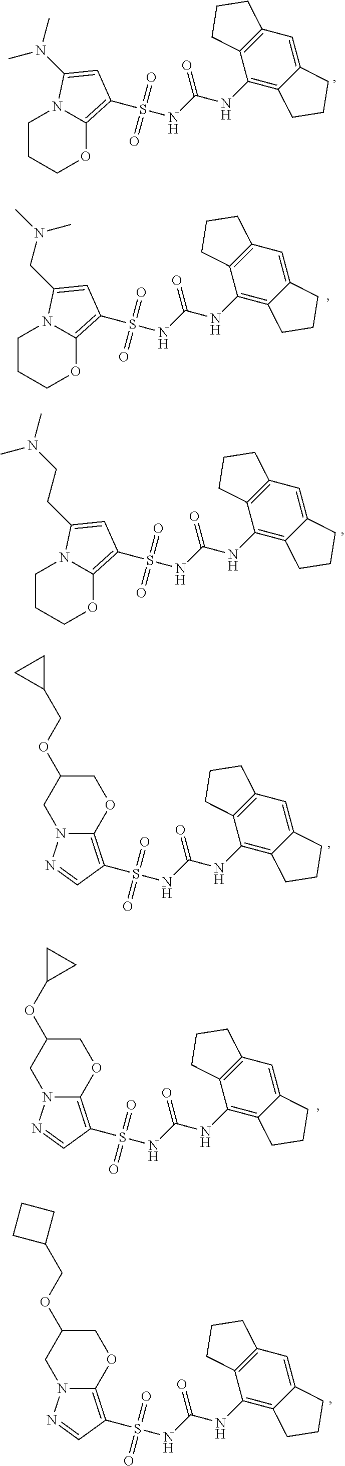

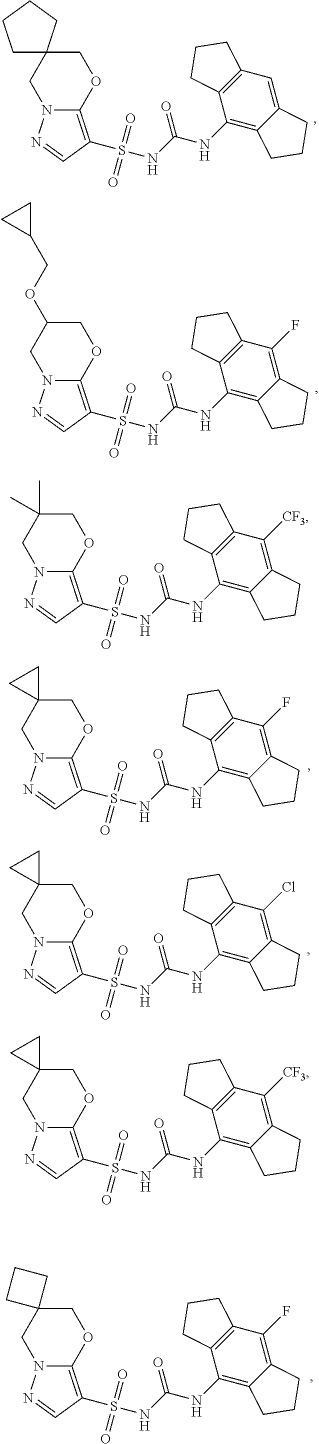

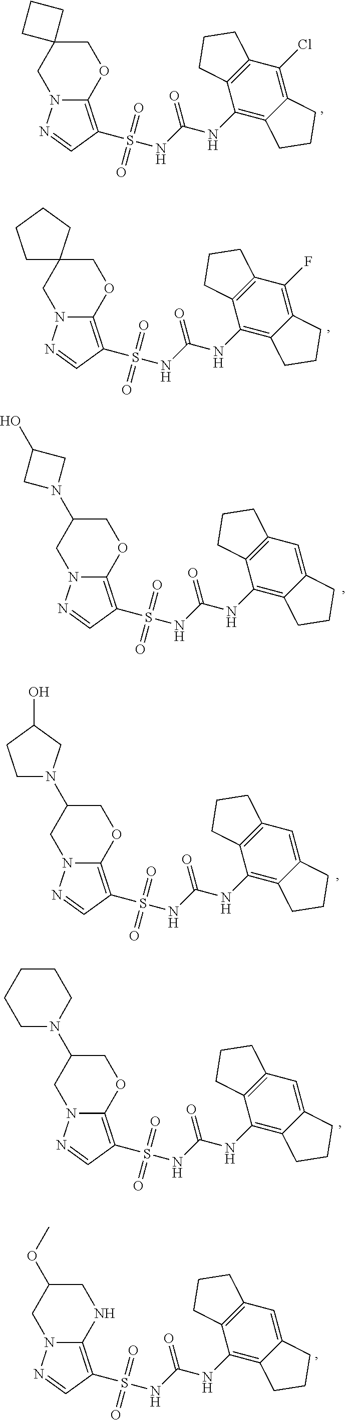 [SMILES: O=C(Nc1c2c(c(Cl)c3c1CCC3)CCC2)NS(=O)(=O)c1cnn2c1OCC1(CCC1)C2 ; O=C(Nc1c2c(c(F)c3c1CCC3)CCC2)NS(=O)(=O)c1cnn2c1OCC1(CCCC1)C2 ; O=C(Nc1c2c(cc3c1CCC3)CCC2)NS(=O)(=O)c1cnn2c1OCC(N1CC(O)C1)C2 ; O=C(Nc1c2c(cc3c1CCC3)CCC2)NS(=O)(=O)c1cnn2c1OCC(N1CCC(O)C1)C2 ; O=C(Nc1c2c(cc3c1CCC3)CCC2)NS(=O)(=O)c1cnn2c1OCC(N1CCCCC1)C2 ; COC1CNc2c(S(=O)(=O)NC(=O)Nc3c4c(cc5c3CCC5)CCC4)cnn2C1]

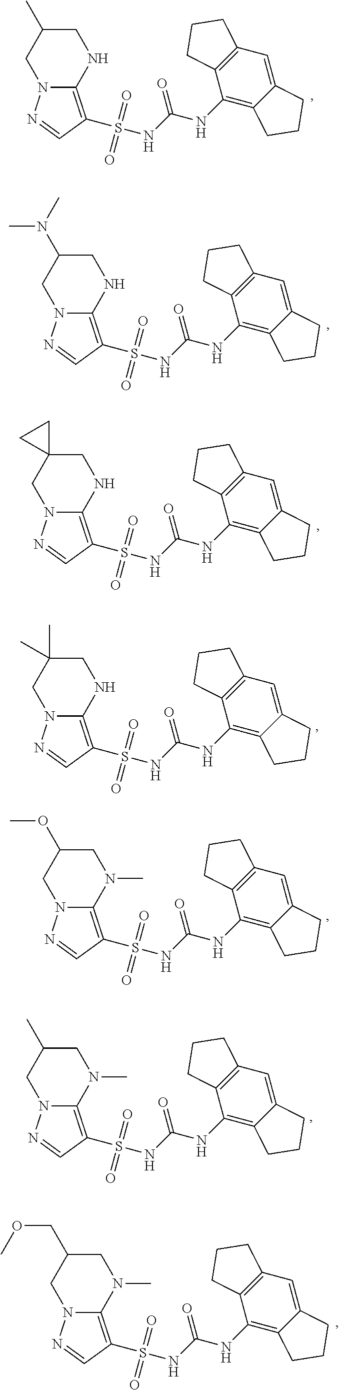

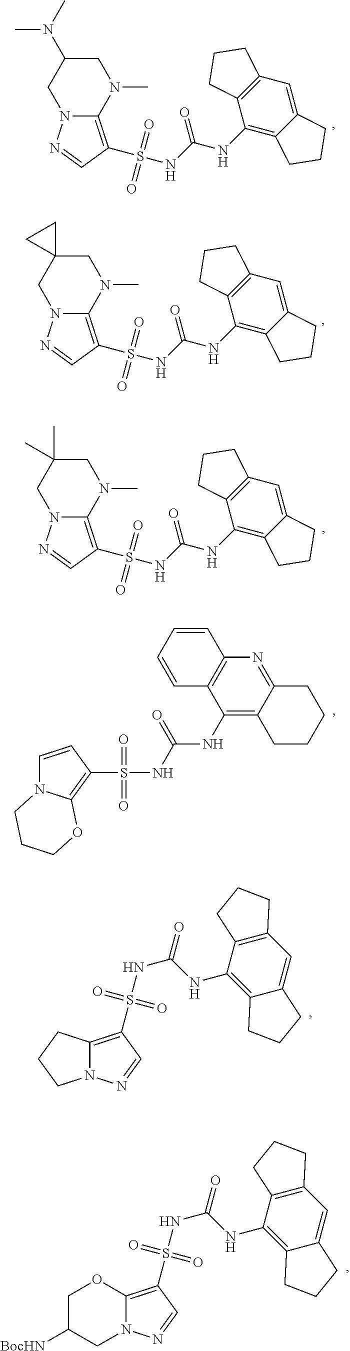 [SMILES: CN1CC(N(C)C)Cn2ncc(S(=O)(=O)NC(=O)Nc3c4c(cc5c3CCC5)CCC4)c21 ; CN1CC2(CC2)Cn2ncc(S(=O)(=O)NC(=O)Nc3c4c(cc5c3CCC5)CCC4)c21 ; CN1CC(C)(C)Cn2ncc(S(=O)(=O)NC(=O)Nc3c4c(cc5c3CCC5)CCC4)c21 ; O=C(Nc1c2c(nc3ccccc13)CCCC2)NS(=O)(=O)c1ccn2c1OCCC2 ; O=C(Nc1c2c(cc3c1CCC3)CCC2)NS(=O)(=O)c1cnn2c1CCC2 ; CC(C)(C)OC(=O)NC1COc2c(S(=O)(=O)NC(=O)Nc3c4c(cc5c3CCC5)CCC4)cnn2C1]

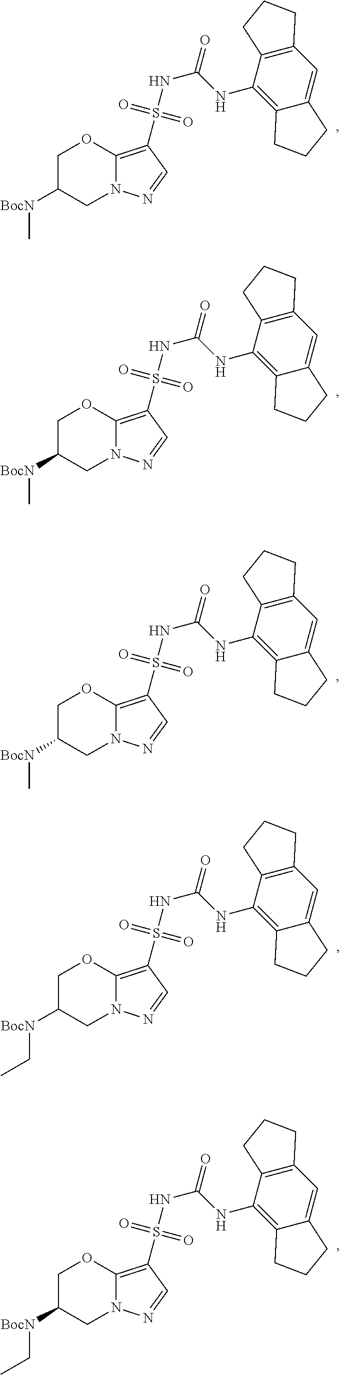 [SMILES: CN(C(=O)OC(C)(C)C)C1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(cc5c3CCC5)CCC4)cnn2C1 ; CN(C(=O)OC(C)(C)C)[C@@H]1COc2c(S(=O)(=O)NC(=O)Nc3c4c(cc5c3CCC5)CCC4)cnn2C1 ; CCN(C(=O)OC(C)(C)C)C1COc2c(S(=O)(=O)NC(=O)Nc3c4c(cc5c3CCC5)CCC4)cnn2C1 ; CCN(C(=O)OC(C)(C)C)[C@H]1COc2c(S(=O)(=O)NC(=O)Nc3c4c(cc5c3CCC5)CCC4)cnn2C1]

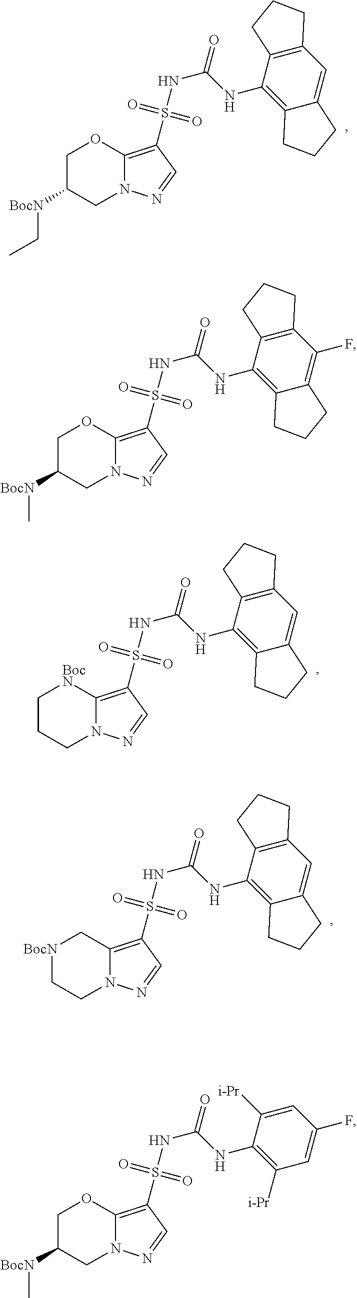 [SMILES: CCN(C(=O)OC(C)(C)C)[C@@H]1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(c(F)c5c3CCC5)CCC4)cnn2C1 ; CC(C)(C)OC(=O)N1CCCn2ncc(S(=O)(=O)NC(=O)Nc3c4c(cc5c3CCC5)CCC4)c21 ; CC(C)(C)OC(=O)N1CCn2ncc(S(=O)(=O)NC(=O)Nc3c4c(cc5c3CCC5)CCC4)c2C1 ; CC(C)c1cc(F)cc(C(C)C)c1NC(=O)NS(=O)(=O)c1cnn2c1OC[C@H](N(C)C(=O)OC(C)(C)C)C2]

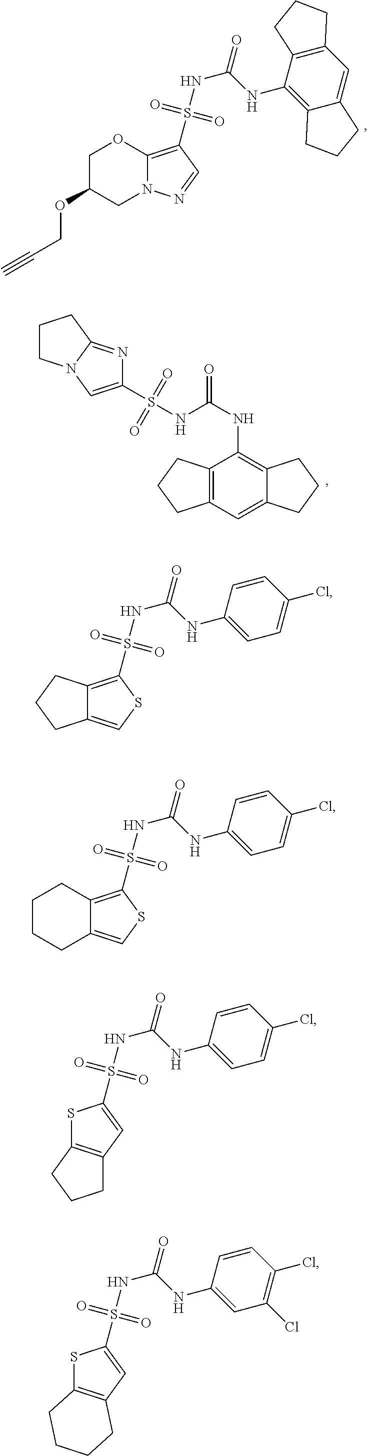 [SMILES: C#CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cn2c(n1)CCC2 ; O=C(Nc1ccc(Cl)cc1)NS(=O)(=O)c1scc2c1CCC2 ; O=C(Nc1ccc(Cl)cc1)NS(=O)(=O)c1scc2c1CCCC2 ; O=C(Nc1ccc(Cl)cc1)NS(=O)(=O)c1cc2c(s1)CCC2 ; O=C(Nc1ccc(Cl)c(Cl)c1)NS(=O)(=O)c1cc2c(s1)CCCC2]

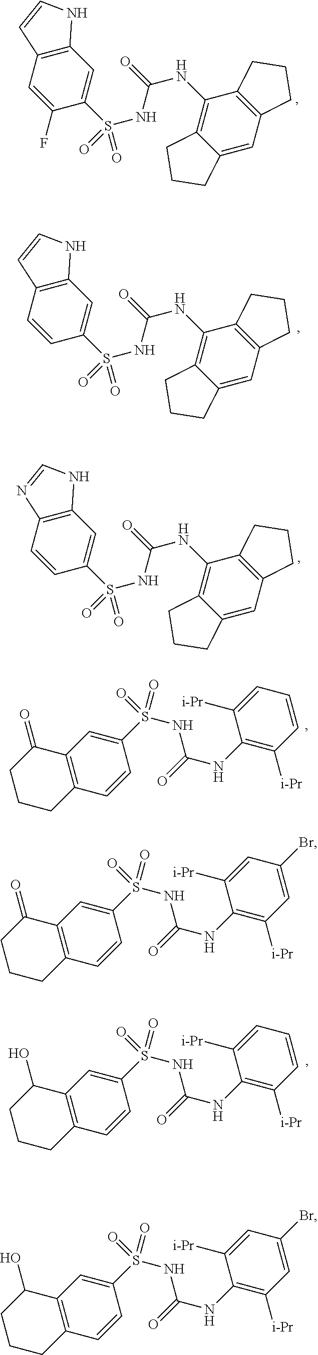

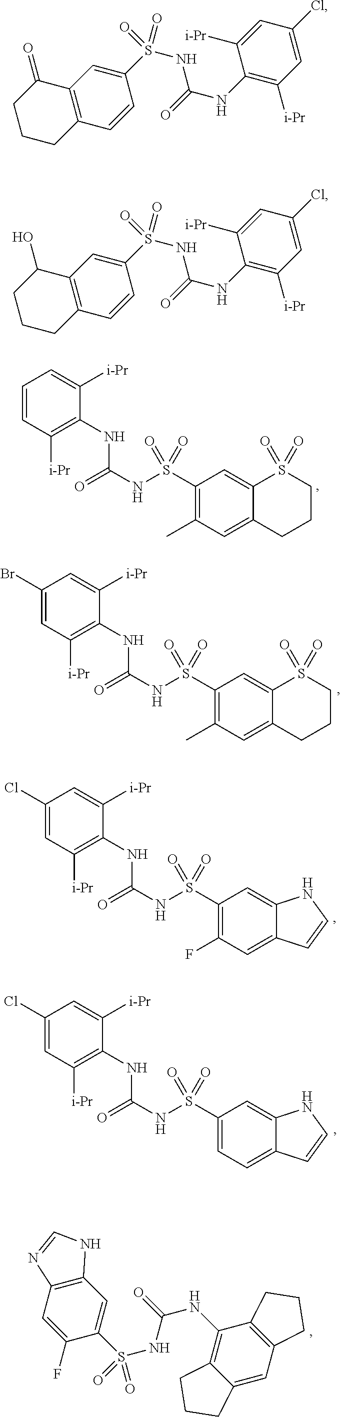

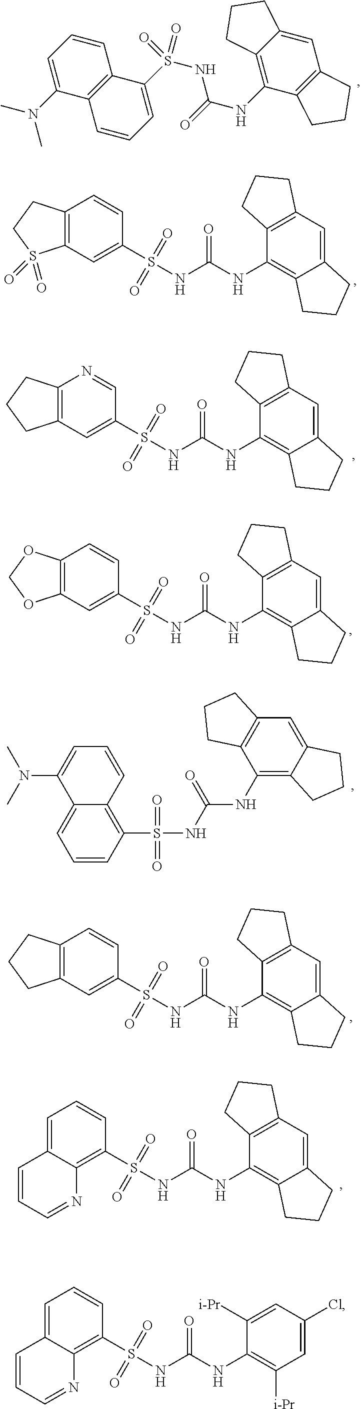

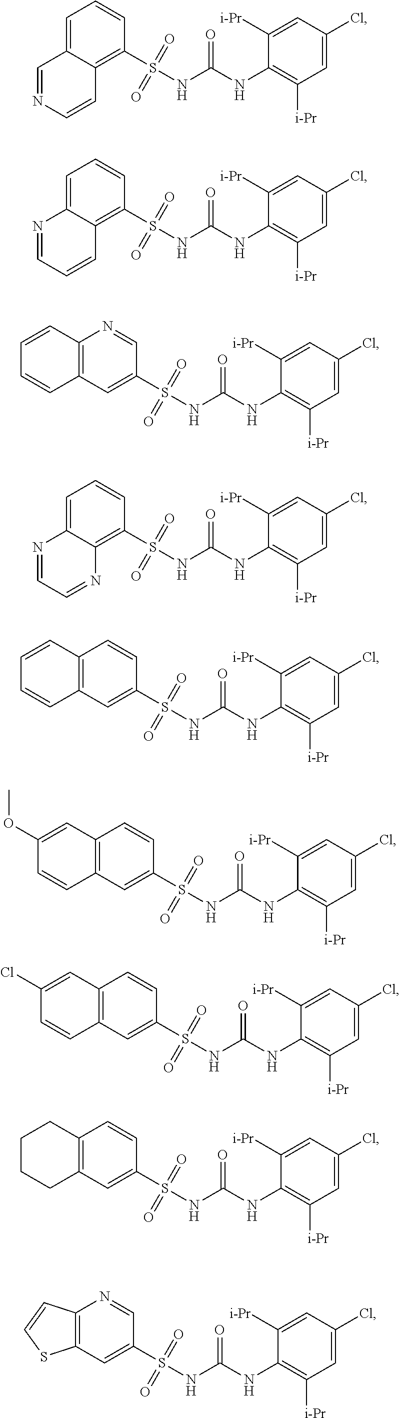

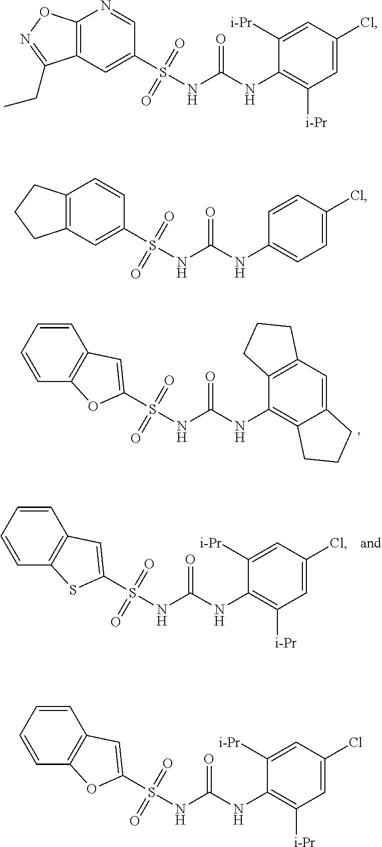 [SMILES: CCc1noc2ncc(S(=O)(=O)NC(=O)Nc3c(C(C)C)cc(Cl)cc3C(C)C)cc12 ; O=C(Nc1ccc(Cl)cc1)NS(=O)(=O)c1ccc2c(c1)CCC2 ; O=C(Nc1c2c(cc3c1CCC3)CCC2)NS(=O)(=O)c1cc2ccccc2o1 ; CC(C)c1cc(Cl)cc(C(C)C)c1NC(=O)NS(=O)(=O)c1cc2ccccc2s1 ; "and" ; CC(C)c1cc(Cl)cc(C(C)C)c1NC(=O)NS(=O)(=O)c1cc2ccccc2o1]

or a pharmaceutically acceptable salt thereof.

In one aspect, provided herein is a compound of Formula AA

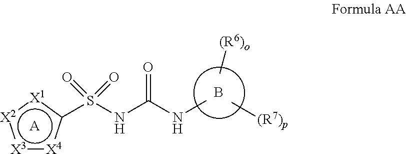

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
when X⁴ is —X⁵—X⁶, then: X¹ is N or CR¹, X² is N or CR², and X³ is N or CR³;
when X⁴ is other than —X⁵—X⁶—, then

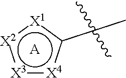

comprises at least one of CR¹, CR², CR³, and CR⁴;
when X⁴ is —X⁵—X⁶—, then

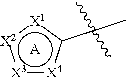

comprises at least two of CR¹, CR², CR³, CR⁵, and CR⁶;
from two to four of R¹, R², R³, and R⁴ are present or from two to five of R¹, R², R³, R⁵, and R⁶ are present; and
wherein at least two of the two to four R¹, R², R³, and R⁴ or at least of the two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰;

R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹;
or at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;
R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
wherein any of R¹, R², R³, R⁴, R⁵, and R⁶ that are not taken together with the atoms connecting them to form a ring, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;
R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;
R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
B is a 5-10-membered heteroaryl or C₆-C₁₀ aryl ring;
o=1 or 2;
p=0, 1, 2, or 3;
R⁶ and R⁷ are each independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵,
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
provided that when B is 5-10-membered heteroaryl with from 2-3 ring nitrogen atoms, at least one R⁶ is bonded to B at a position ortho to the —HNC(═O)NHS(O)₂— moiety of Formula AA;
when B is 2-pyridyl, pyrimidin-6-yl, or pyrimidin-4-yl, B is not substituted with a cyano group at a position ortho to the —HNC(═O)NHS(O)₂— moiety of Formula AA;
and
with the proviso that the compound of Formula AA is not a compound selected from the group consisting of:

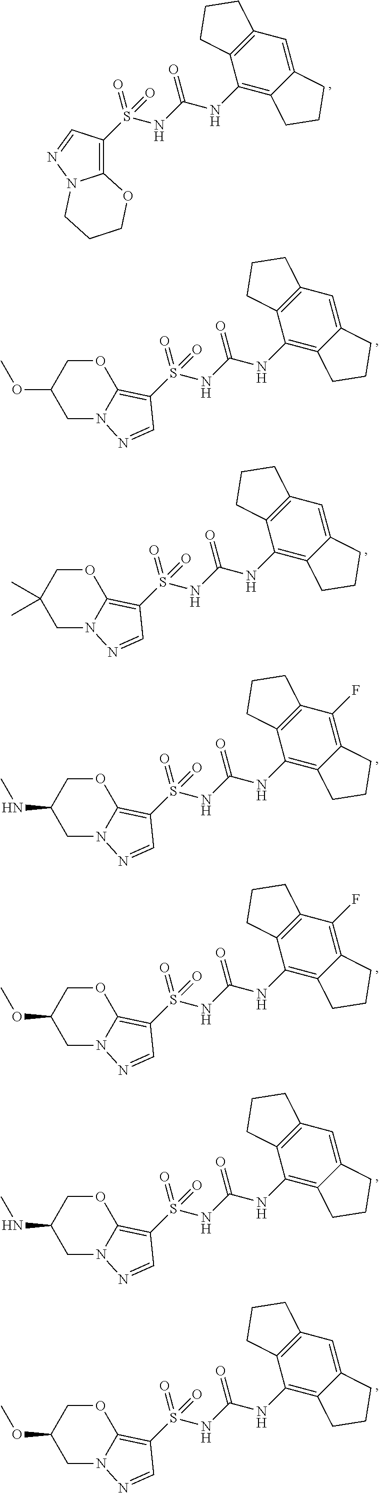

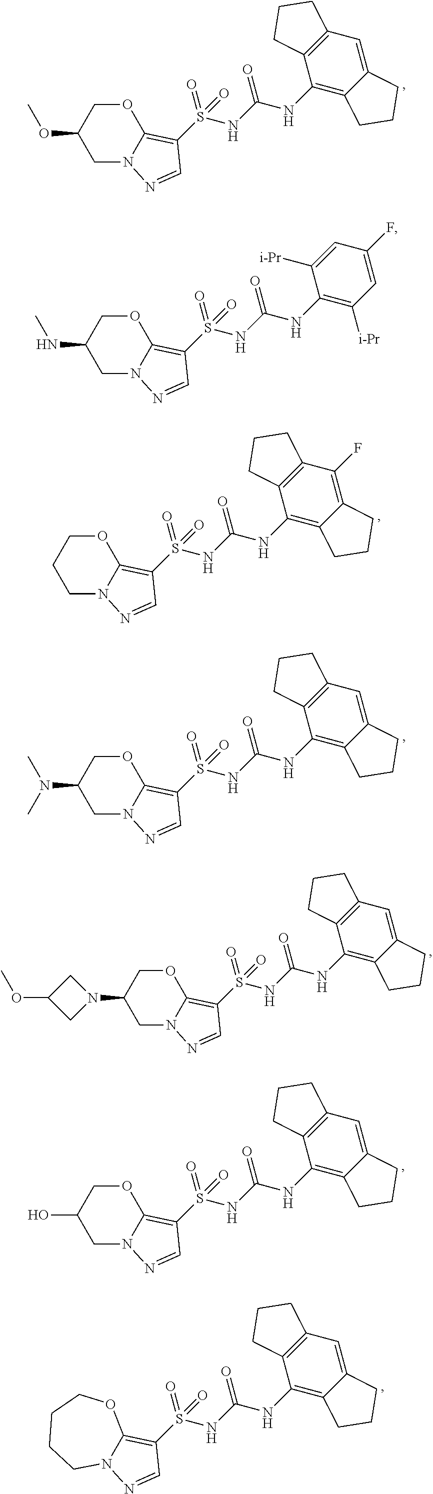

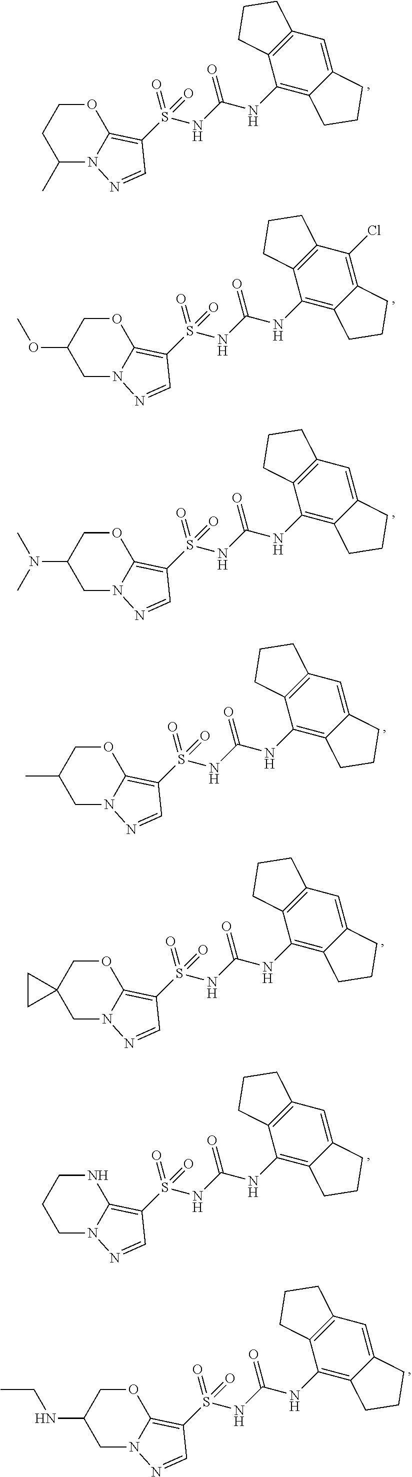

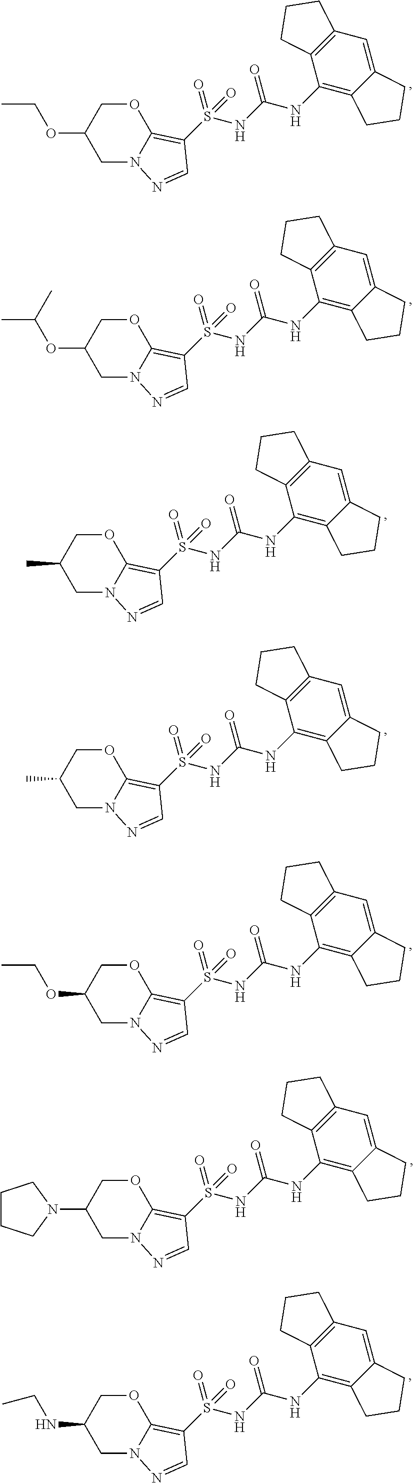

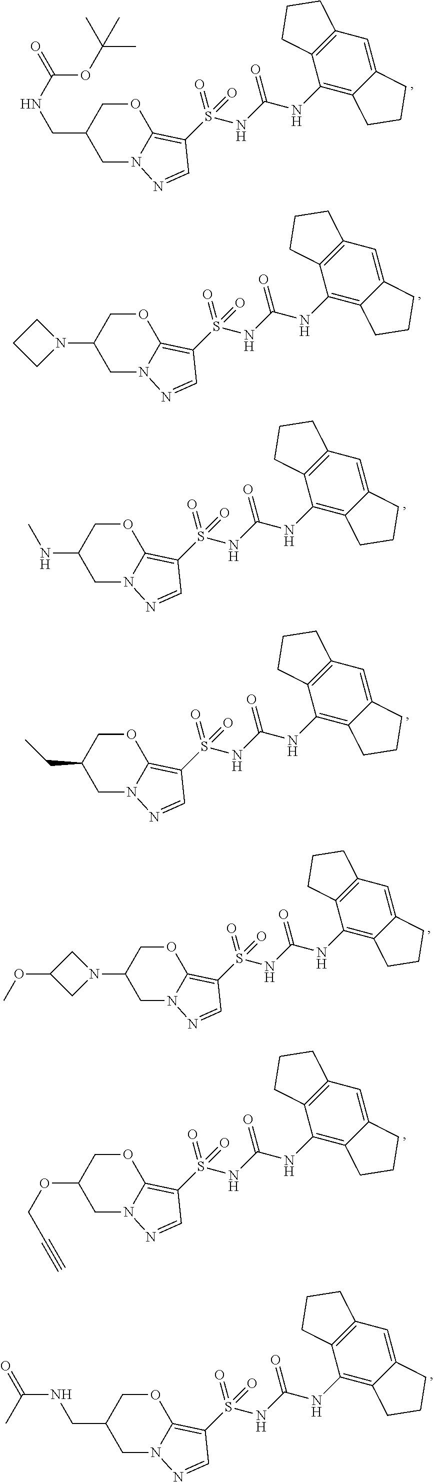

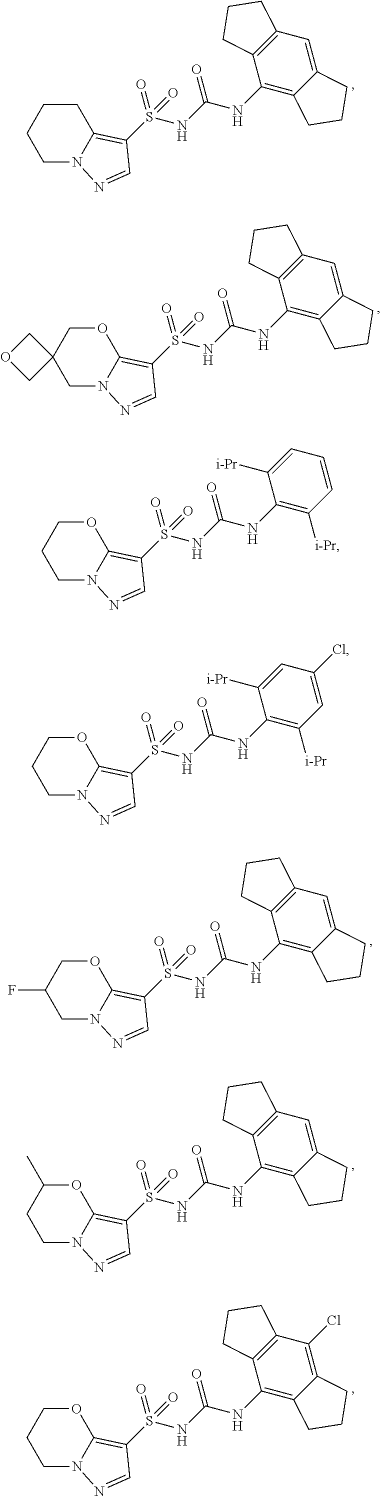

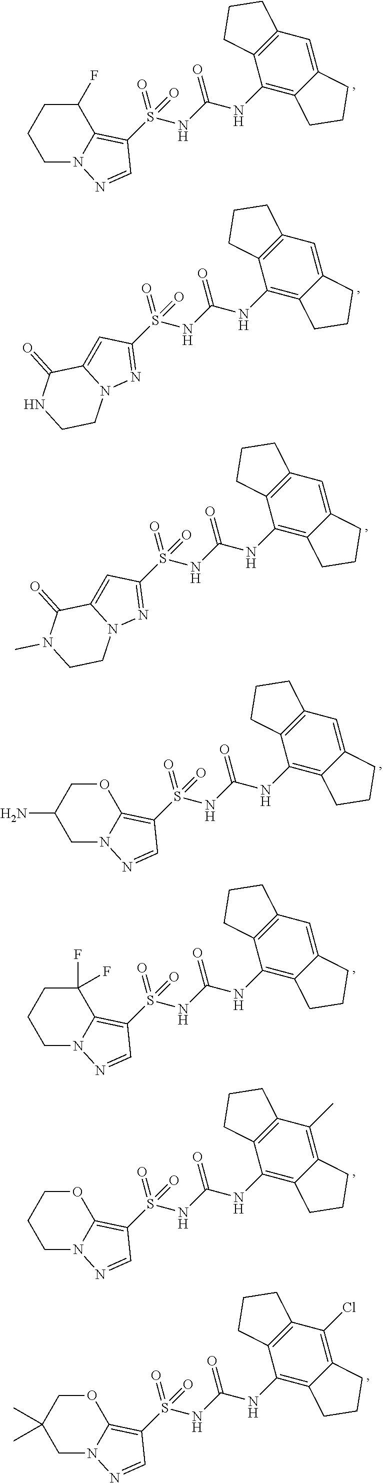

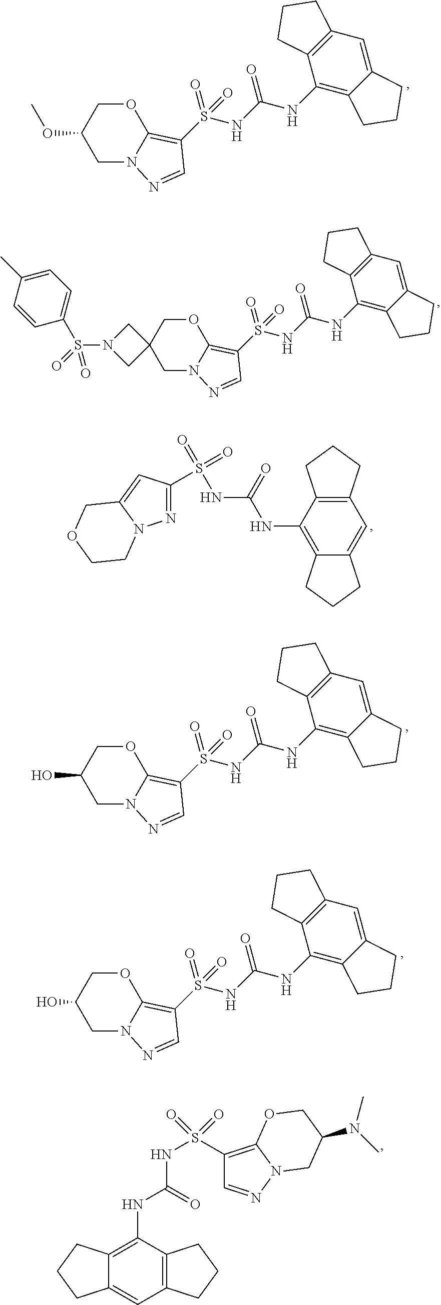

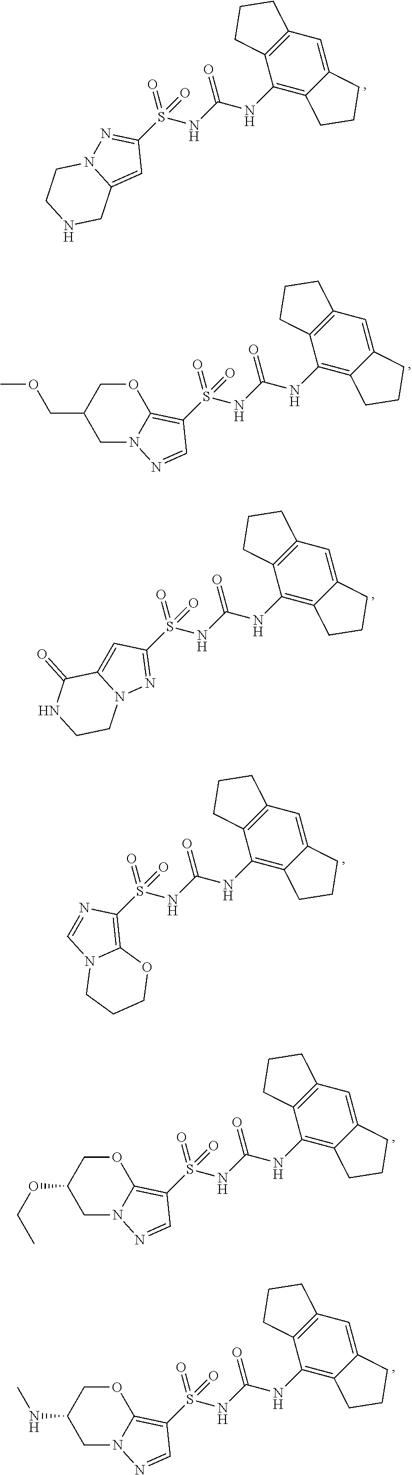 [SMILES: O=C(Nc1c2c(cc3c1CCC3)CCC2)NS(=O)(=O)c1cc2n(n1)CCNC2 ; COCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cc2n(n1)CCNC2=O ; O=C(Nc1c2c(cc3c1CCC3)CCC2)NS(=O)(=O)c1ncn2c1OCCC2 ; CCO[C@@H]1COc2c(S(=O)(=O)NC(=O)Nc3c4c(cc5c3CCC5)CCC4)cnn2C1 ; CN[C@@H]1COc2c(S(=O)(=O)NC(=O)Nc3c4c(cc5c3CCC5)CCC4)cnn2C1]

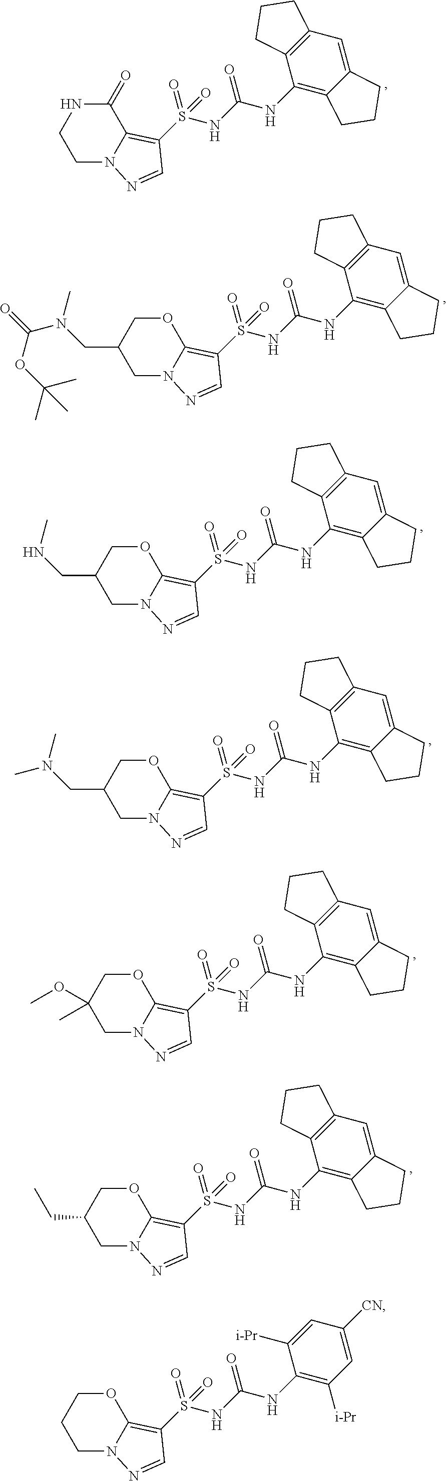

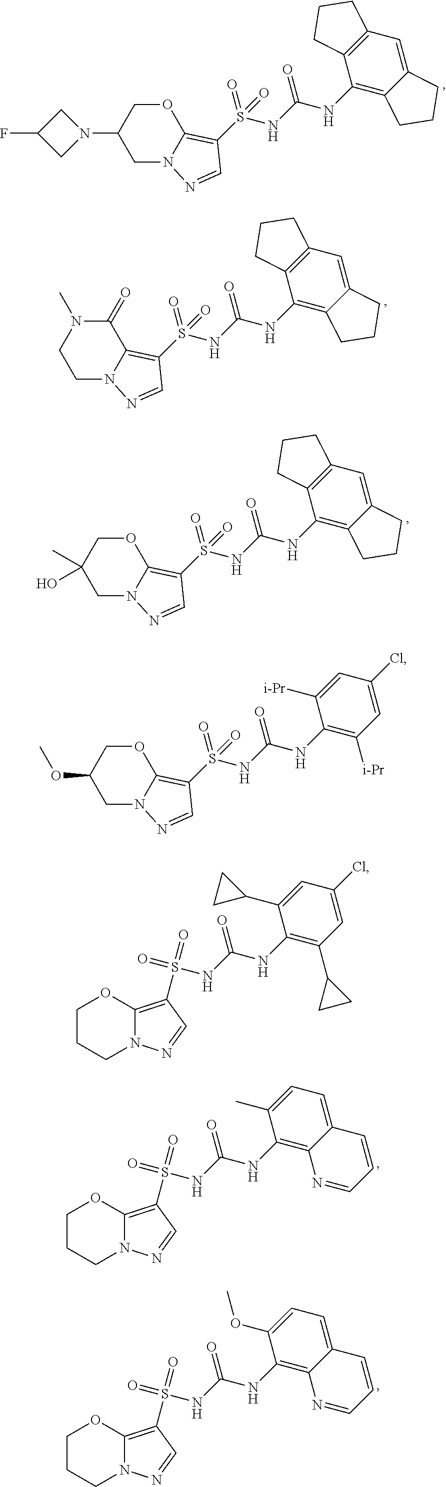

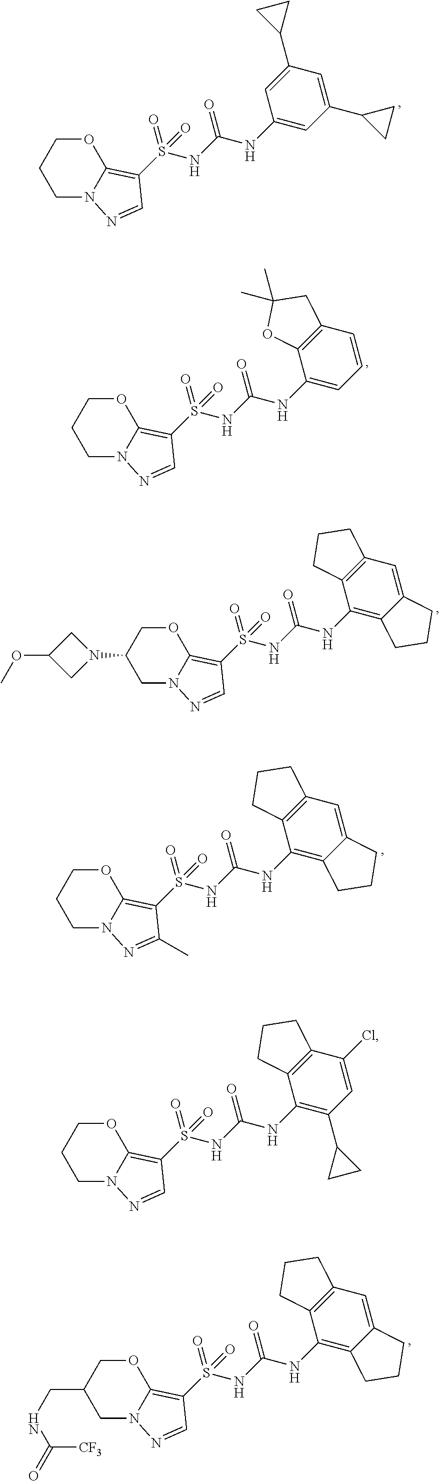 [SMILES: O=C(Nc1cc(C2CC2)cc(C2CC2)c1)NS(=O)(=O)c1cnn2c1OCCC2 ; CC1(C)Cc2cccc(NC(=O)NS(=O)(=O)c3cnn4c3OCCC4)c2O1 ; COC1CN([C@@H]2COc3c(S(=O)(=O)NC(=O)Nc4c5c(cc6c4CCC6)CCC5)cnn3C2)C1 ; Cc1nn2c(c1S(=O)(=O)NC(=O)Nc1c3c(cc4c1CCC4)CCC3)OCCC2 ; O=C(Nc1c(C2CC2)cc(Cl)c2c1CCC2)NS(=O)(=O)c1cnn2c1OCCC2 ; O=C(Nc1c2c(cc3c1CCC3)CCC2)NS(=O)(=O)c1cnn2c1OCC(CNC(=O)C(F)(F)F)C2]

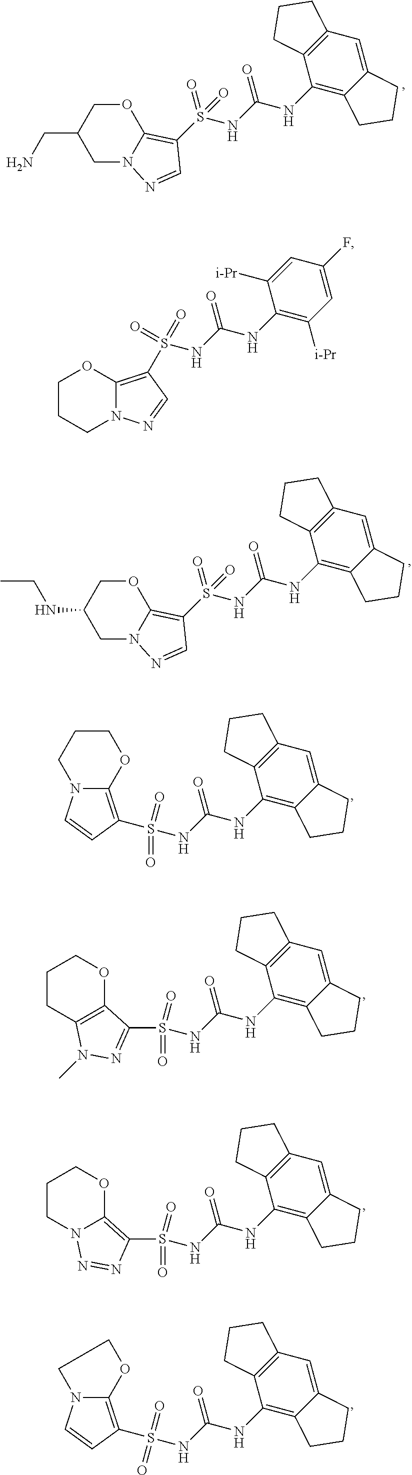

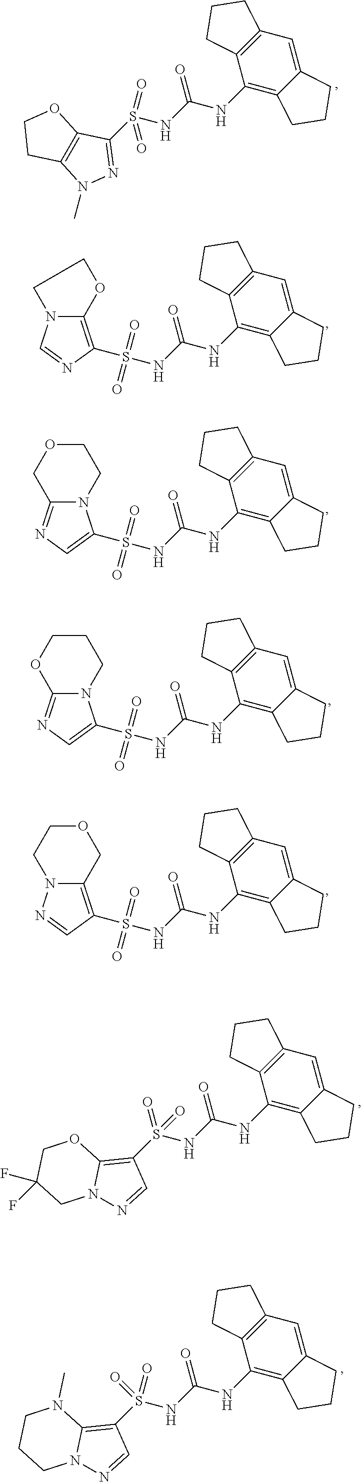

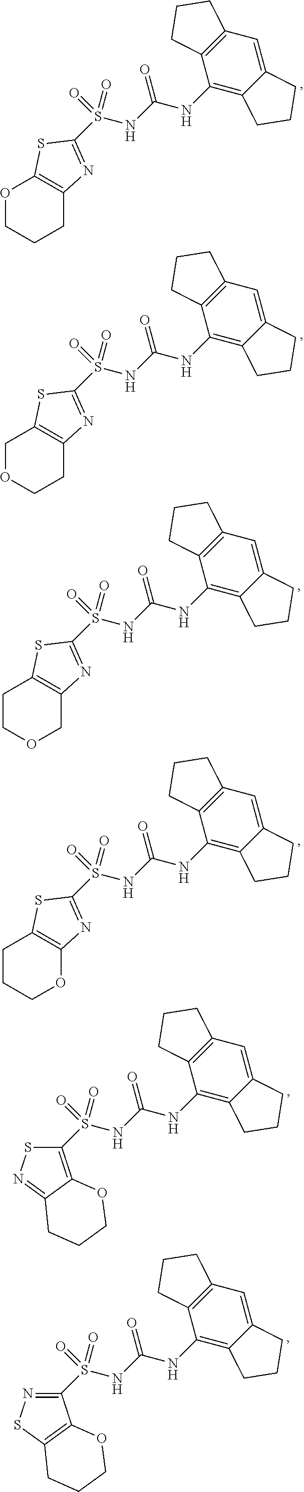 [SMILES: O=C(Nc1c2c(cc3c1CCC3)CCC2)NS(=O)(=O)c1nc2c(s1)OCCC2 ; O=C(Nc1c2c(cc3c1CCC3)CCC2)NS(=O)(=O)c1nc2c(s1)COCC2 ; O=C(Nc1c2c(cc3c1CCC3)CCC2)NS(=O)(=O)c1nc2c(s1)CCOC2 ; O=C(Nc1c2c(cc3c1CCC3)CCC2)NS(=O)(=O)c1nc2c(s1)CCCO2 ; O=C(Nc1c2c(cc3c1CCC3)CCC2)NS(=O)(=O)c1snc2c1OCCC2 ; O=C(Nc1c2c(cc3c1CCC3)CCC2)NS(=O)(=O)c1nsc2c1OCCC2]

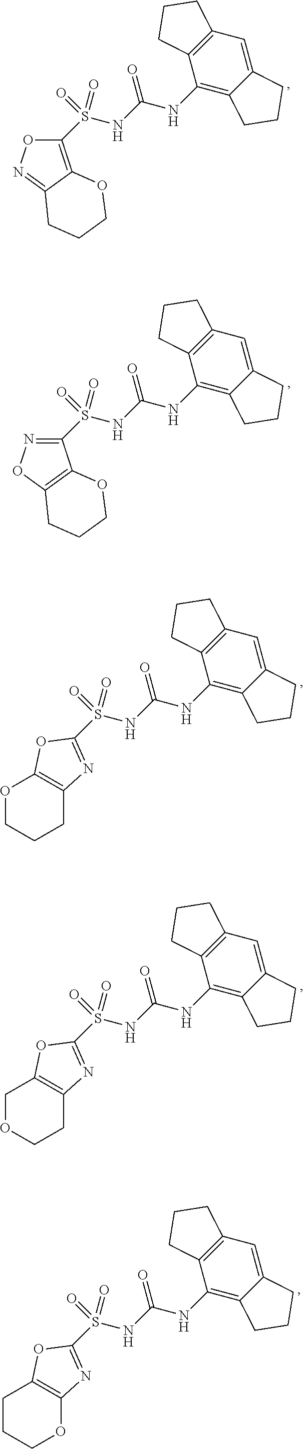 [SMILES: O=C(Nc1c2c(cc3c1CCC3)CCC2)NS(=O)(=O)c1onc2c1OCCC2 ; O=C(Nc1c2c(cc3c1CCC3)CCC2)NS(=O)(=O)c1noc2c1OCCC2 ; O=C(Nc1c2c(cc3c1CCC3)CCC2)NS(=O)(=O)c1nc2c(o1)OCCC2 ; O=C(Nc1c2c(cc3c1CCC3)CCC2)NS(=O)(=O)c1nc2c(o1)COCC2 ; O=C(Nc1c2c(cc3c1CCC3)CCC2)NS(=O)(=O)c1nc2c(o1)CCCO2]

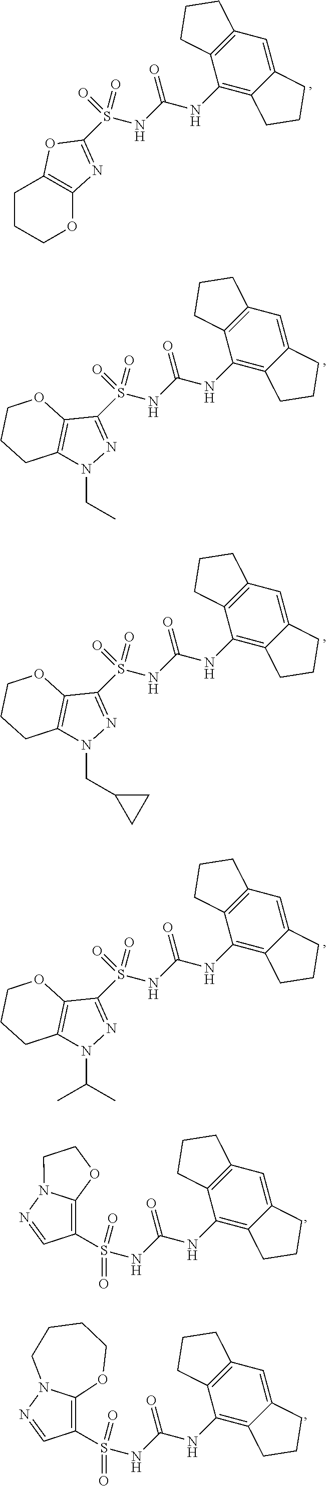

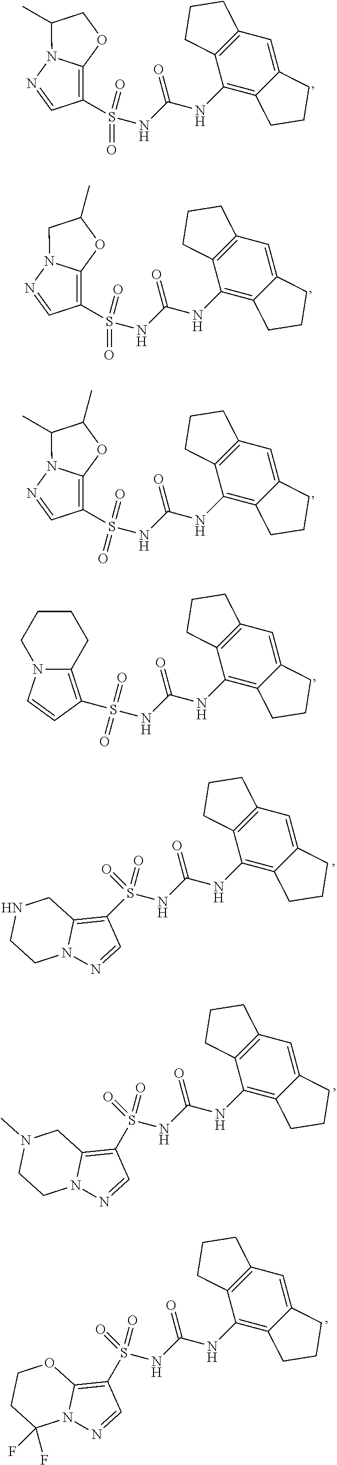

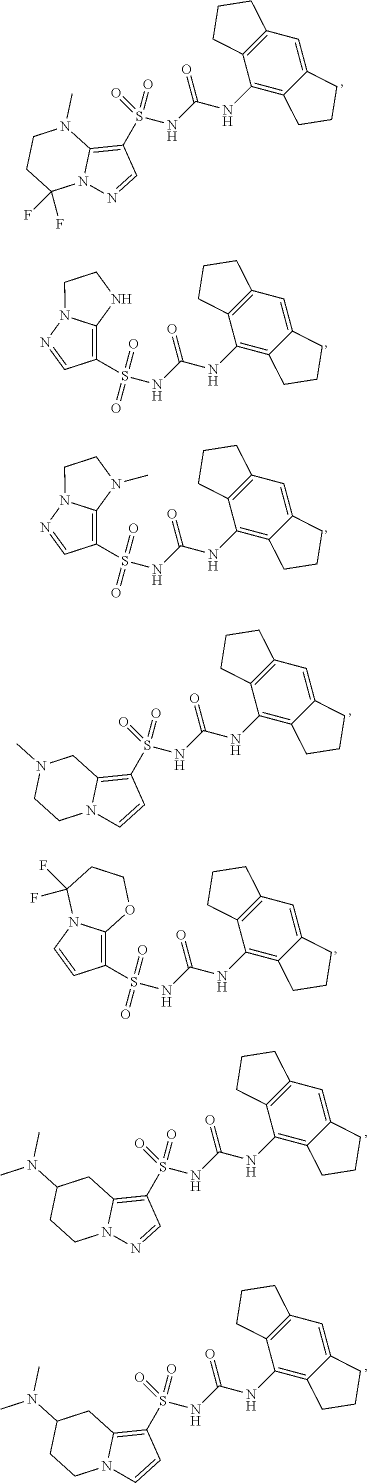

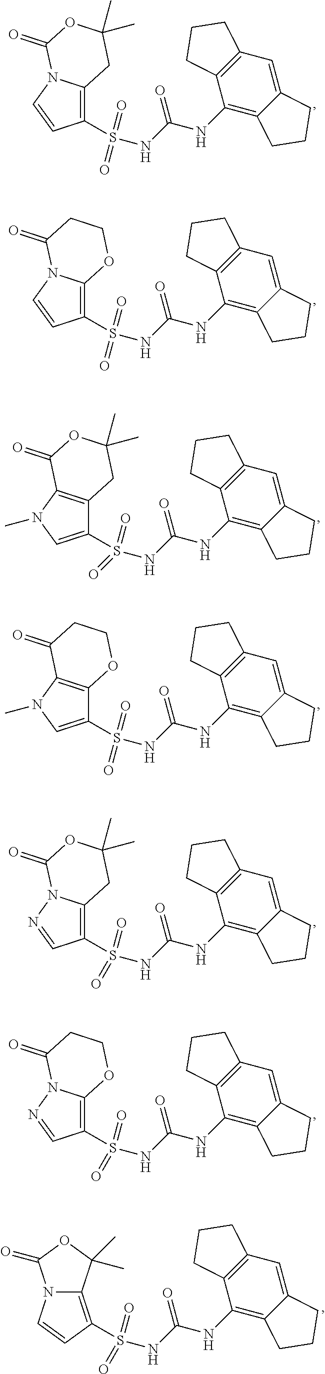

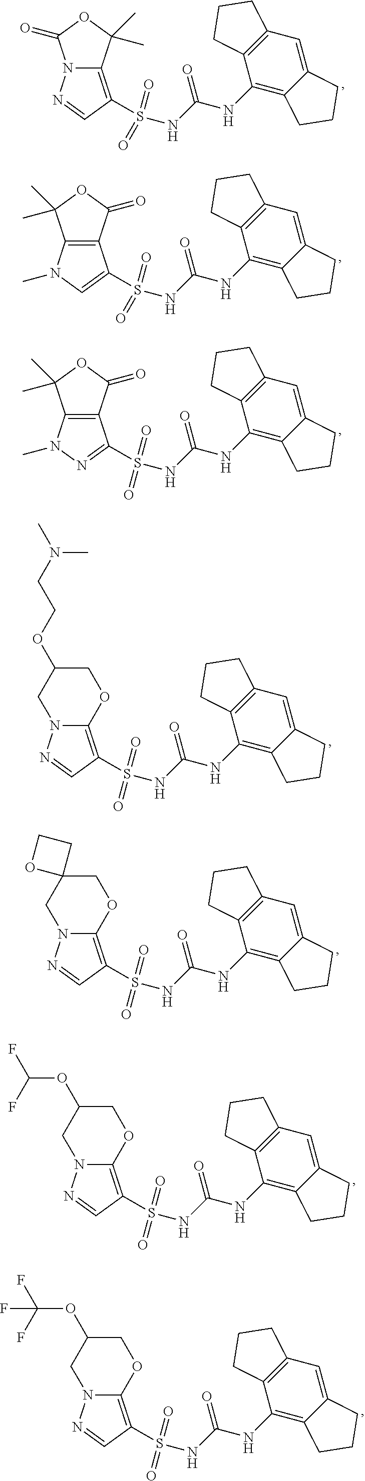

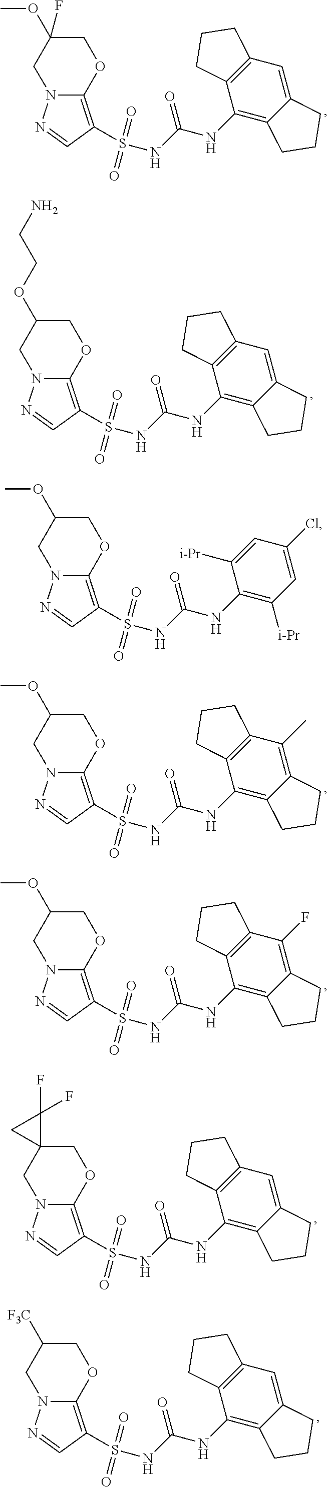

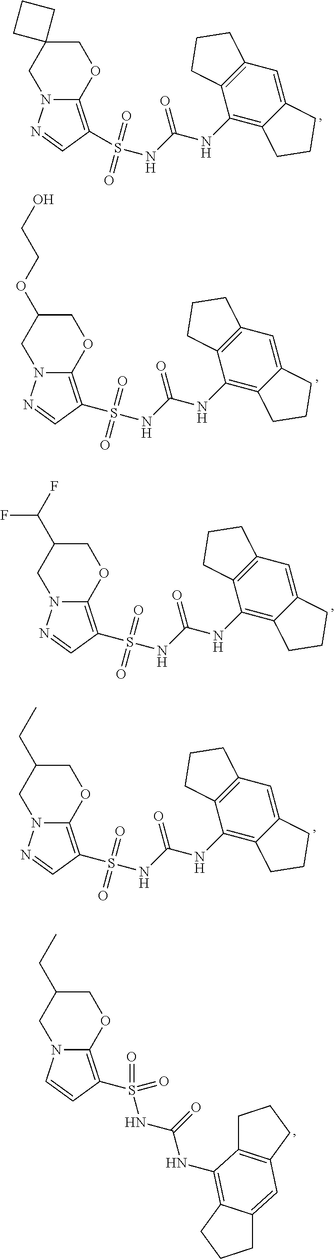 [SMILES: O=C(Nc1c2c(cc3c1CCC3)CCC2)NS(=O)(=O)c1cnn2c1OCC1(CCC1)C2 ; O=C(Nc1c2c(cc3c1CCC3)CCC2)NS(=O)(=O)c1cnn2c1OCC(OCCO)C2 ; O=C(Nc1c2c(cc3c1CCC3)CCC2)NS(=O)(=O)c1cnn2c1OCC(C(F)F)C2 ; CCC1COc2c(S(=O)(=O)NC(=O)Nc3c4c(cc5c3CCC5)CCC4)cnn2C1 ; CCC1COc2c(S(=O)(=O)NC(=O)Nc3c4c(cc5c3CCC5)CCC4)ccn2C1]

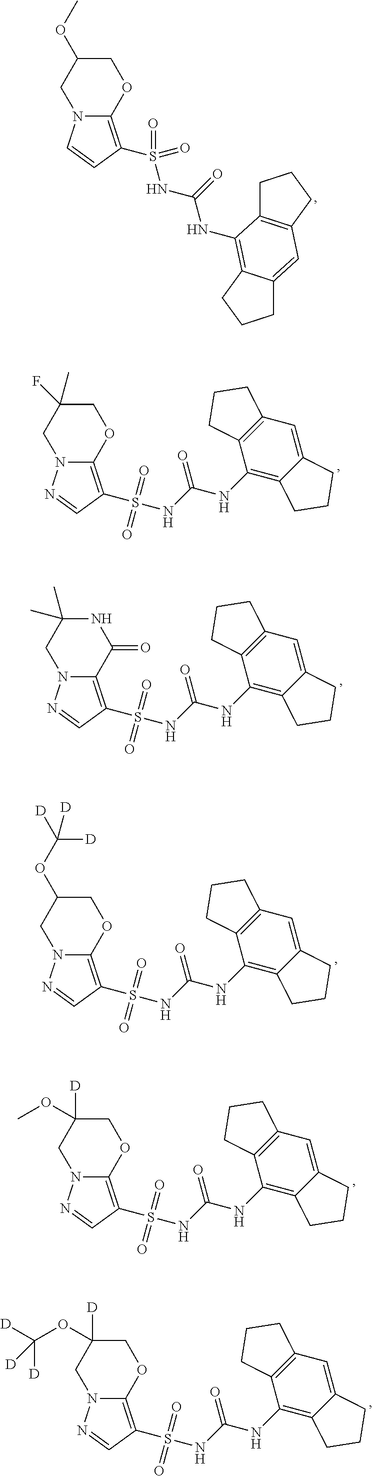 [SMILES: COC1COc2c(S(=O)(=O)NC(=O)Nc3c4c(cc5c3CCC5)CCC4)ccn2C1 ; CC1(F)COc2c(S(=O)(=O)NC(=O)Nc3c4c(cc5c3CCC5)CCC4)cnn2C1 ; CC1(C)Cn2ncc(S(=O)(=O)NC(=O)Nc3c4c(cc5c3CCC5)CCC4)c2C(=O)N1 ; [2H]C([2H])([2H])OC1COc2c(S(=O)(=O)NC(=O)Nc3c4c(cc5c3CCC5)CCC4)cnn2C1 ; [2H]C1(OC)COc2c(S(=O)(=O)NC(=O)Nc3c4c(cc5c3CCC5)CCC4)cnn2C1 ; [2H]C([2H])([2H])OC1([2H])COc2c(S(=O)(=O)NC(=O)Nc3c4c(cc5c3CCC5)CCC4)cnn2C1]

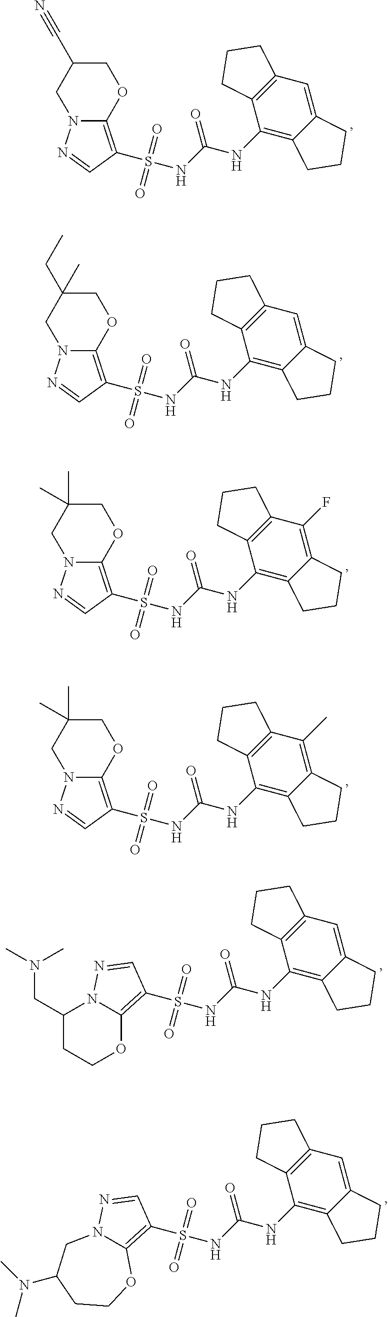 [SMILES: N#CC1COc2c(S(=O)(=O)NC(=O)Nc3c4c(cc5c3CCC5)CCC4)cnn2C1 ; CCC1(C)COc2c(S(=O)(=O)NC(=O)Nc3c4c(cc5c3CCC5)CCC4)cnn2C1 ; CC1(C)COc2c(S(=O)(=O)NC(=O)Nc3c4c(c(F)c5c3CCC5)CCC4)cnn2C1 ; Cc1c2c(c(NC(=O)NS(=O)(=O)c3cnn4c3OCC(C)(C)C4)c3c1CCC3)CCC2 ; CN(C)CC1CCOc2c(S(=O)(=O)NC(=O)Nc3c4c(cc5c3CCC5)CCC4)cnn21 ; CN(C)C1CCOc2c(S(=O)(=O)NC(=O)Nc3c4c(cc5c3CCC5)CCC4)cnn2C1]

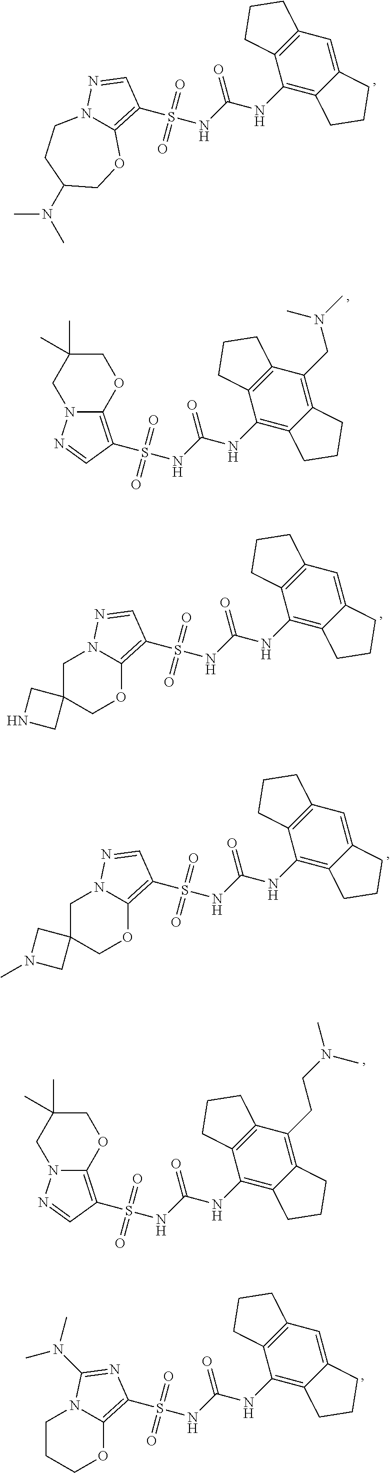

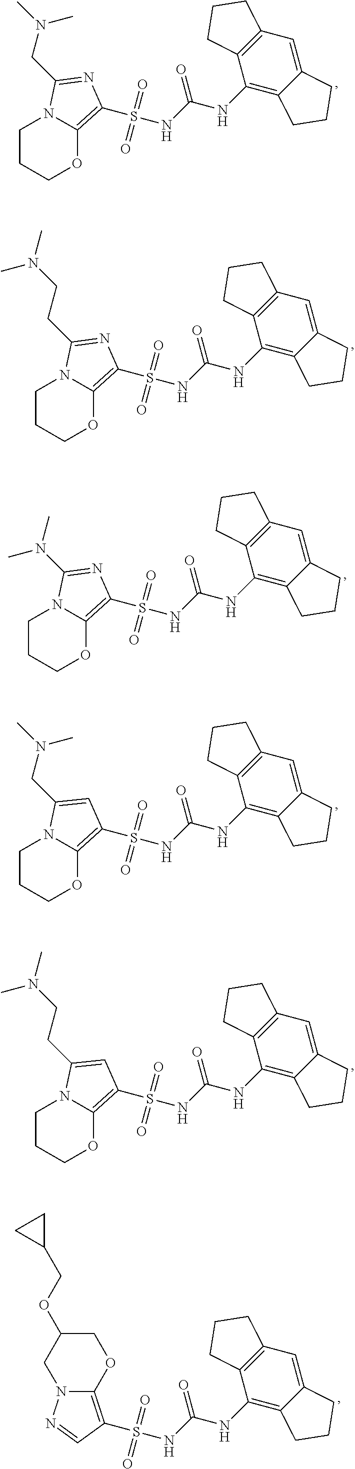 [SMILES: CN(C)Cc1nc(S(=O)(=O)NC(=O)Nc2c3c(cc4c2CCC4)CCC3)c2n1CCCO2 ; CN(C)CCc1nc(S(=O)(=O)NC(=O)Nc2c3c(cc4c2CCC4)CCC3)c2n1CCCO2 ; CN(C)c1nc(S(=O)(=O)NC(=O)Nc2c3c(cc4c2CCC4)CCC3)c2n1CCCO2 ; CN(C)Cc1cc(S(=O)(=O)NC(=O)Nc2c3c(cc4c2CCC4)CCC3)c2n1CCCO2 ; CN(C)CCc1cc(S(=O)(=O)NC(=O)Nc2c3c(cc4c2CCC4)CCC3)c2n1CCCO2 ; O=C(Nc1c2c(cc3c1CCC3)CCC2)NS(=O)(=O)c1cnn2c1OCC(OCC1CC1)C2]

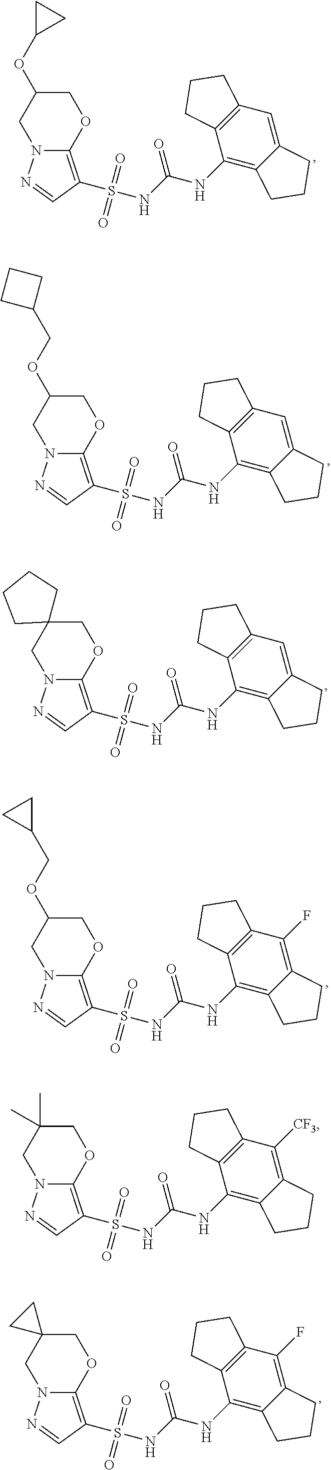 [SMILES: O=C(Nc1c2c(cc3c1CCC3)CCC2)NS(=O)(=O)c1cnn2c1OCC(OC1CC1)C2 ; O=C(Nc1c2c(cc3c1CCC3)CCC2)NS(=O)(=O)c1cnn2c1OCC(OCC1CCC1)C2 ; O=C(Nc1c2c(cc3c1CCC3)CCC2)NS(=O)(=O)c1cnn2c1OCC1(CCCC1)C2 ; O=C(Nc1c2c(c(F)c3c1CCC3)CCC2)NS(=O)(=O)c1cnn2c1OCC(OCC1CC1)C2 ; CC1(C)COc2c(S(=O)(=O)NC(=O)Nc3c4c(c(C(F)(F)F)c5c3CCC5)CCC4)cnn2C1 ; O=C(Nc1c2c(c(F)c3c1CCC3)CCC2)NS(=O)(=O)c1cnn2c1OCC1(CC1)C2]

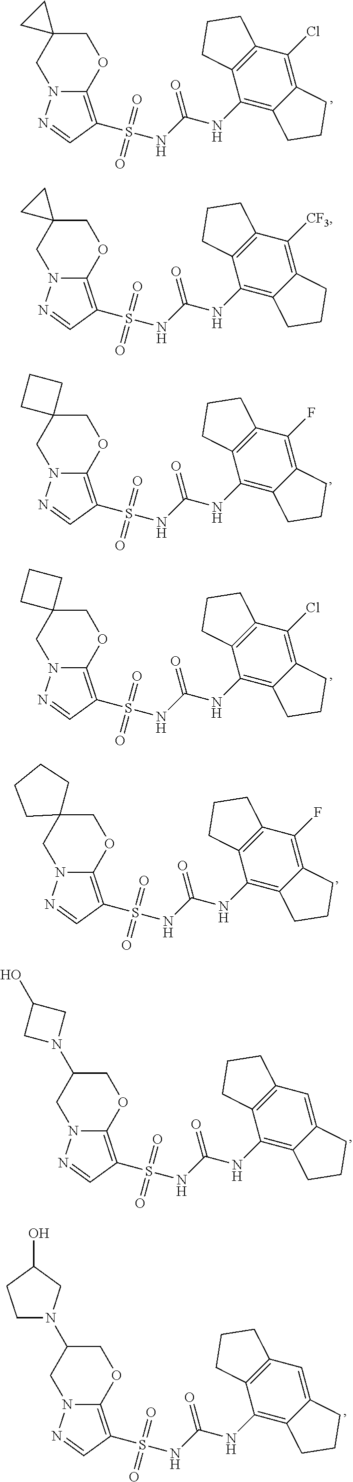

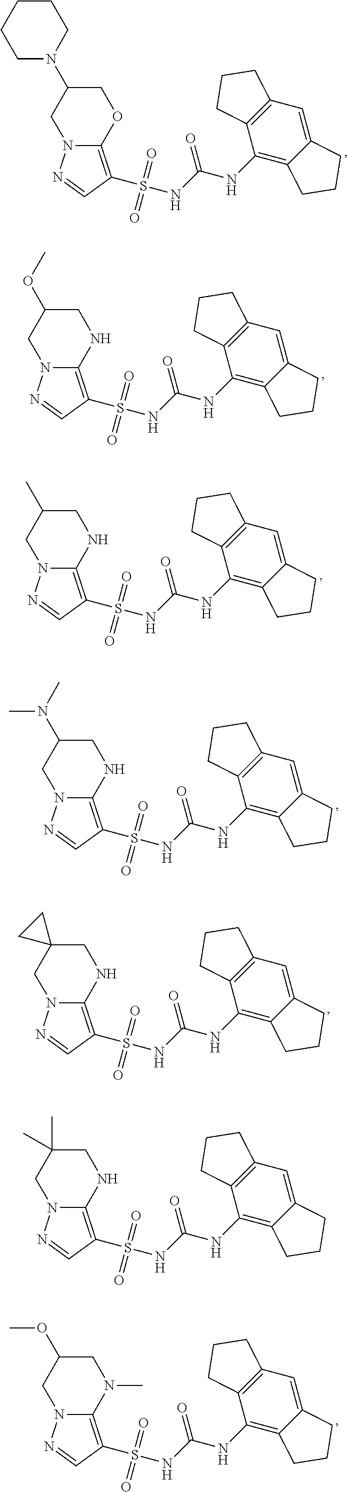

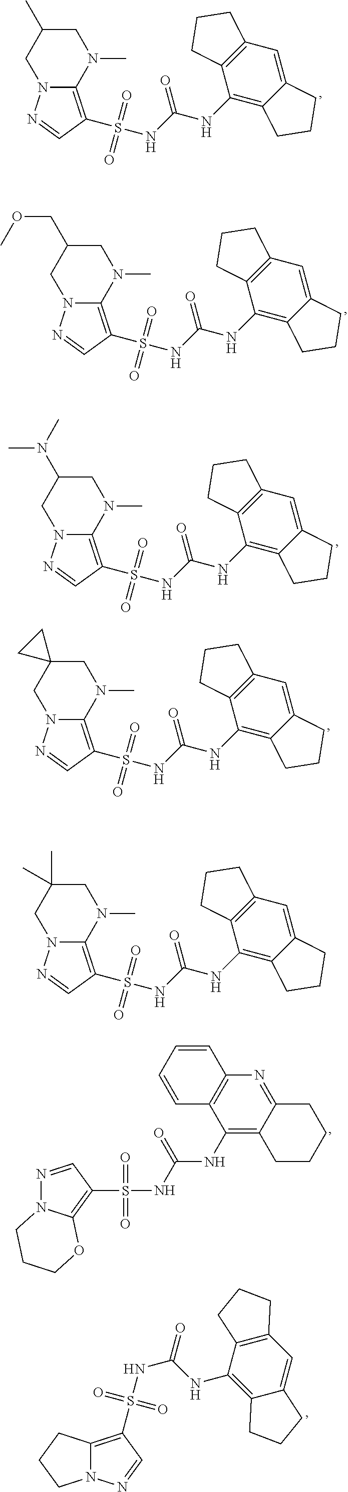

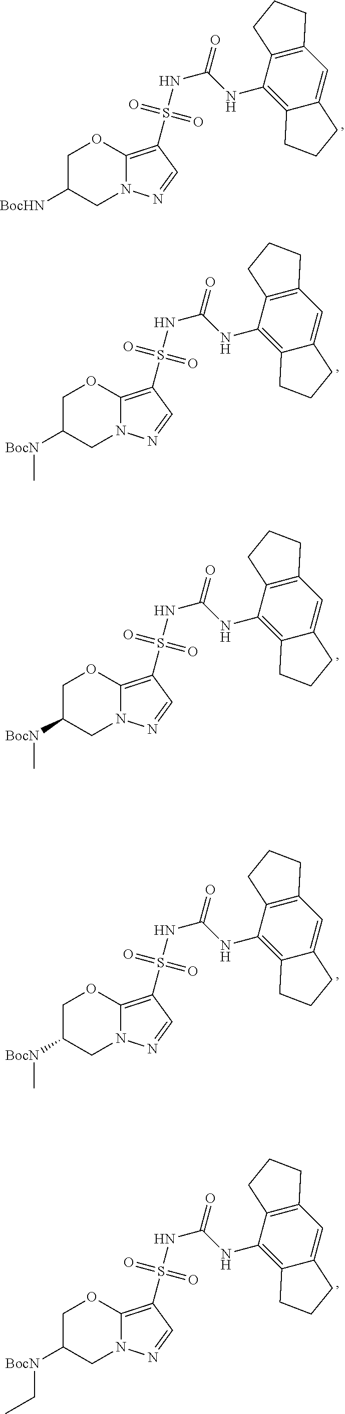 [SMILES: CC(C)(C)OC(=O)NC1COc2c(S(=O)(=O)NC(=O)Nc3c4c(cc5c3CCC5)CCC4)cnn2C1 ; CN(C(=O)OC(C)(C)C)C1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(cc5c3CCC5)CCC4)cnn2C1 ; CN(C(=O)OC(C)(C)C)[C@@H]1COc2c(S(=O)(=O)NC(=O)Nc3c4c(cc5c3CCC5)CCC4)cnn2C1 ; CCN(C(=O)OC(C)(C)C)C1COc2c(S(=O)(=O)NC(=O)Nc3c4c(cc5c3CCC5)CCC4)cnn2C1]

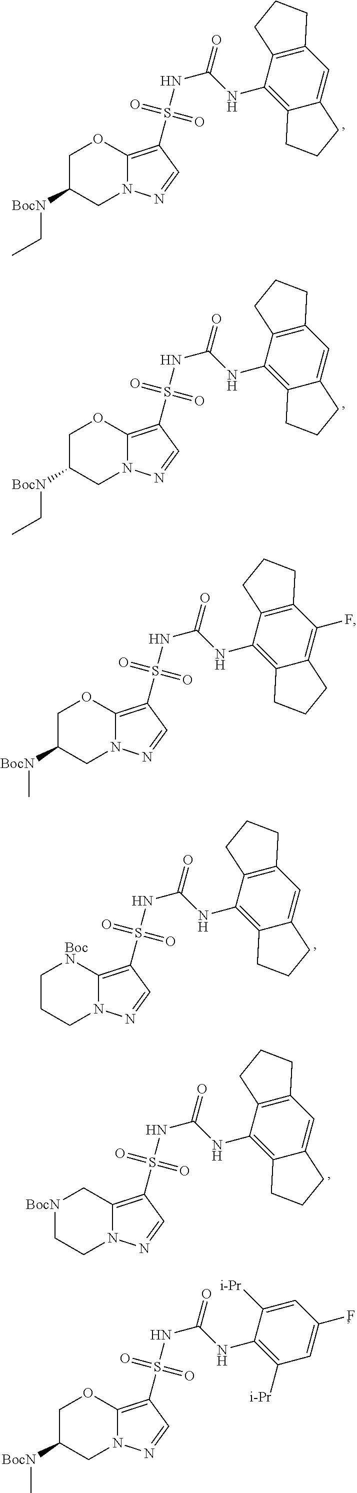 [SMILES: CCN(C(=O)OC(C)(C)C)[C@H]1COc2c(S(=O)(=O)NC(=O)Nc3c4c(cc5c3CCC5)CCC4)cnn2C1 ; CCN(C(=O)OC(C)(C)C)[C@@H]1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(c(F)c5c3CCC5)CCC4)cnn2C1 ; CC(C)(C)OC(=O)N1CCCn2ncc(S(=O)(=O)NC(=O)Nc3c4c(cc5c3CCC5)CCC4)c21 ; CC(C)(C)OC(=O)N1CCn2ncc(S(=O)(=O)NC(=O)Nc3c4c(cc5c3CCC5)CCC4)c2C1 ; CC(C)c1cc(F)cc(C(C)C)c1NC(=O)NS(=O)(=O)c1cnn2c1OC[C@H](N(C)C(=O)OC(C)(C)C)C2]

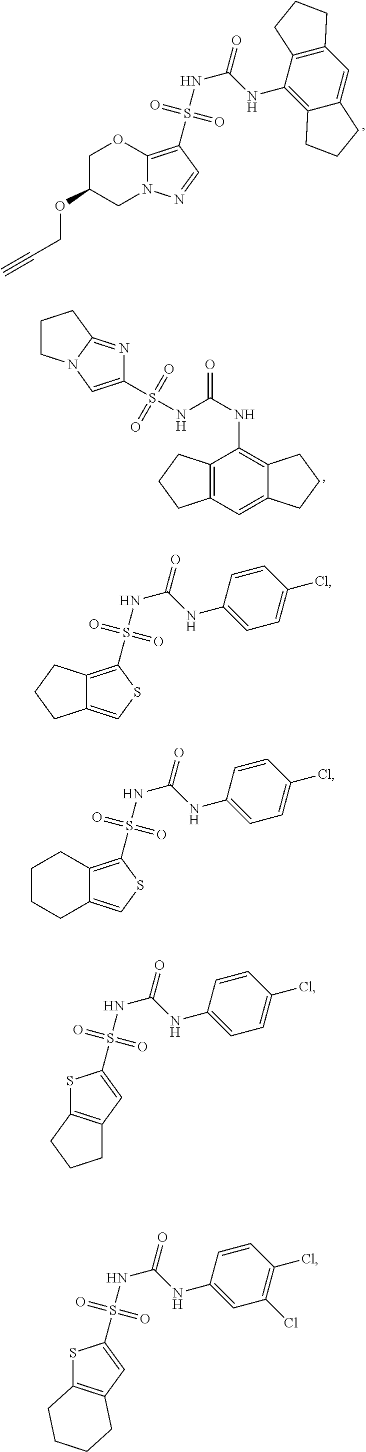 [SMILES: C#CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cn2c(n1)CCC2 ; O=C(Nc1ccc(Cl)cc1)NS(=O)(=O)c1scc2c1CCC2 ; O=C(Nc1ccc(Cl)cc1)NS(=O)(=O)c1scc2c1CCCC2 ; O=C(Nc1ccc(Cl)cc1)NS(=O)(=O)c1cc2c(s1)CCC2 ; O=C(Nc1ccc(Cl)c(Cl)c1)NS(=O)(=O)c1cc2c(s1)CCCC2]

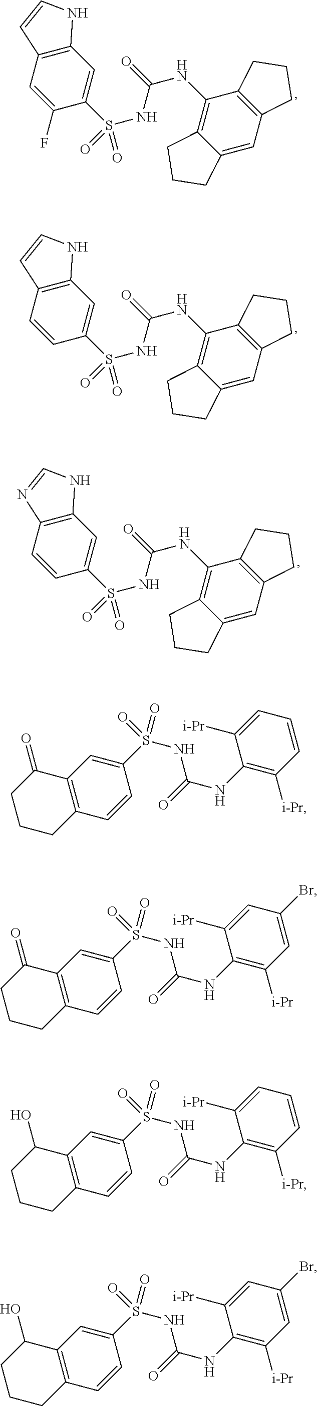

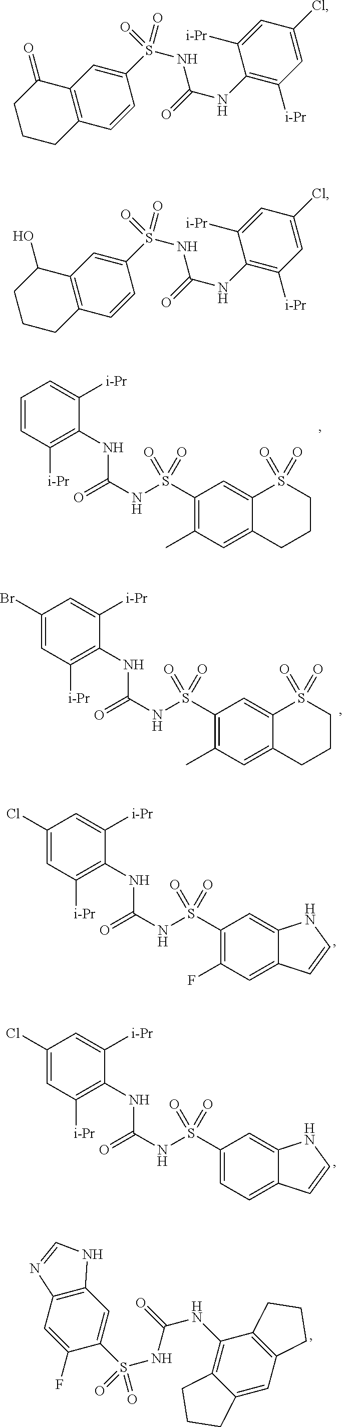

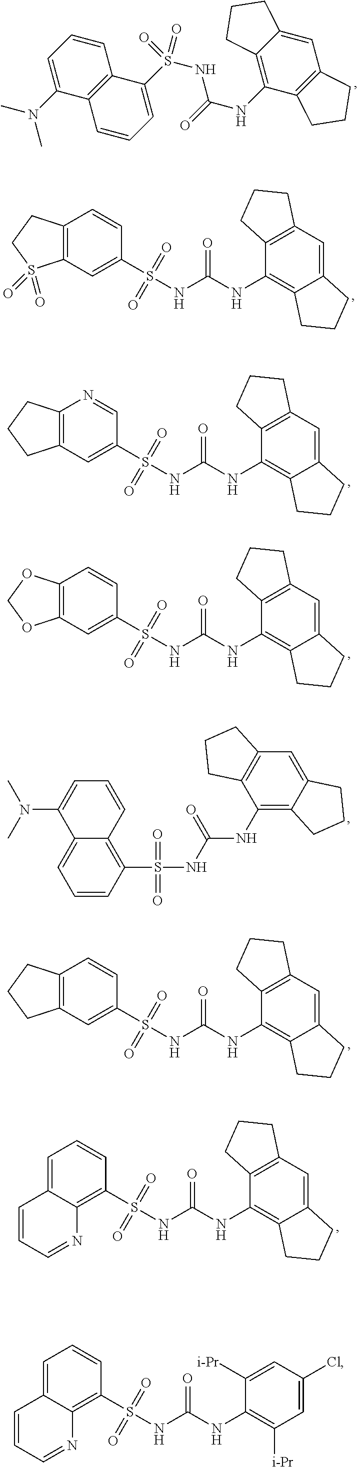

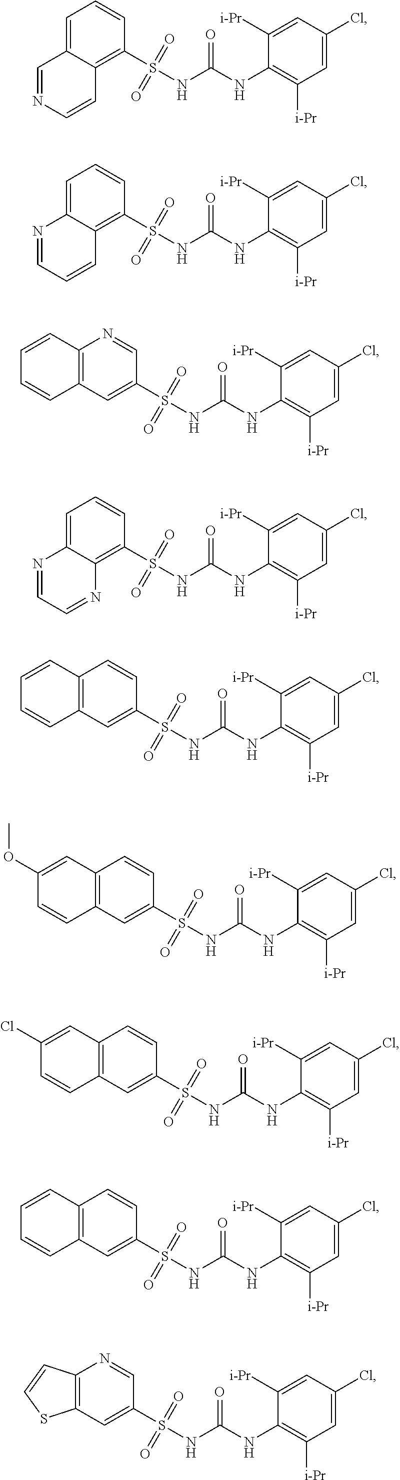

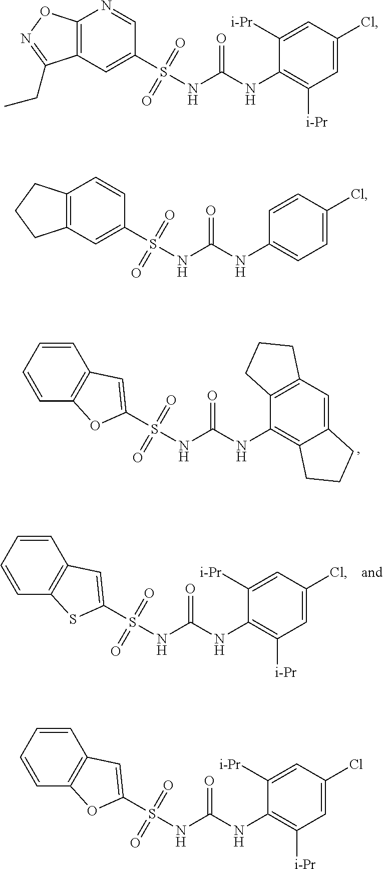 [SMILES: CCc1noc2ncc(S(=O)(=O)NC(=O)Nc3c(C(C)C)cc(Cl)cc3C(C)C)cc12 ; O=C(Nc1ccc(Cl)cc1)NS(=O)(=O)c1ccc2c(c1)CCC2 ; O=C(Nc1c2c(cc3c1CCC3)CCC2)NS(=O)(=O)c1cc2ccccc2o1 ; CC(C)c1cc(Cl)cc(C(C)C)c1NC(=O)NS(=O)(=O)c1cc2ccccc2s1 ; CC(C)c1cc(Cl)cc(C(C)C)c1NC(=O)NS(=O)(=O)c1cc2ccccc2o1]

or a pharmaceutically acceptable salt thereof.

In another aspect, provided herein is a compound of Formula AA

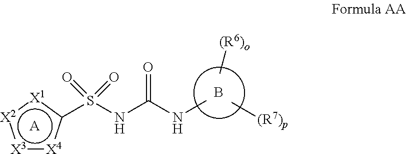

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
when X⁴ is —X⁵—X⁶, then: X¹ is N or CR¹, X² is N or CR², and X³ is N or CR³;
when X⁴ is other than —X⁵—X⁶—, then

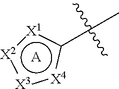

comprises at least one of CR¹, CR², CR³, and CR⁴;
when X⁴ is —X⁵—X⁶—, then

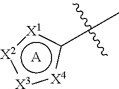

comprises at least two of CR¹, CR², CR³, CR⁵, and CR⁶;
from two to four of R¹, R², R³, and R⁴ are present or from two to five of R¹, R², R³, R⁵, and R⁶ are present; and
wherein at least two of the two to four R¹, R², R³, and R⁴ or at least of two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, NH, NR¹³, and S, wherein the heteroatom or heteroatomic group is cumulative with the values selected for X¹, X², X³, and X⁴, and wherein the heteroaryl ring is optionally substituted with one or more R²⁰;

R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹;
or at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;

or at least one pair of R²⁰ on the adjacent atoms, taken together with the atoms connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;

R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
wherein any of R¹, R², R³, R⁴, R⁵, and R⁶ that are not taken together with the atoms connecting them to form a ring, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;
R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;
R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
B is a 5-10-membered heteroaryl or C₆-C₁₀ aryl ring;
o=1 or 2;
p=0, 1, 2, or 3;
R⁶ and R⁷ are each independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵,
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
provided that when B is 5-10-membered heteroaryl with from 2-3 ring nitrogen atoms, at least one R⁶ is attached to B at a position ortho to the —HNC(═O)NHS(O)₂— moiety of Formula AA;
when B is 2-pyridyl, pyrimidin-6-yl, or pyrimidin-4-yl, B is not substituted with a cyano group at a position ortho to the —HNC(═O)NHS(O)₂— moiety of Formula AA; and
with the proviso that

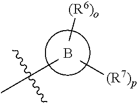

is not selected from the group consisting of:

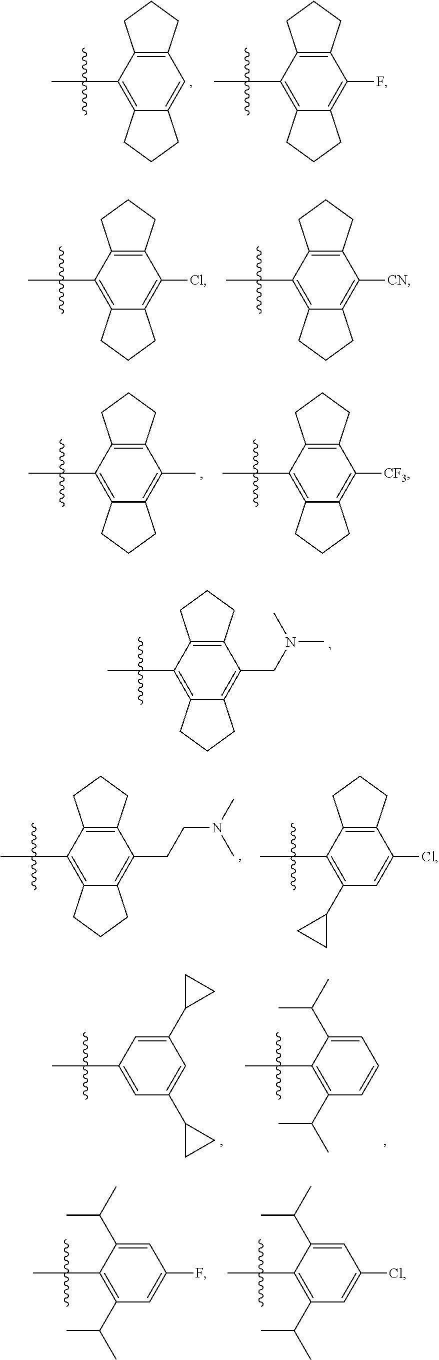

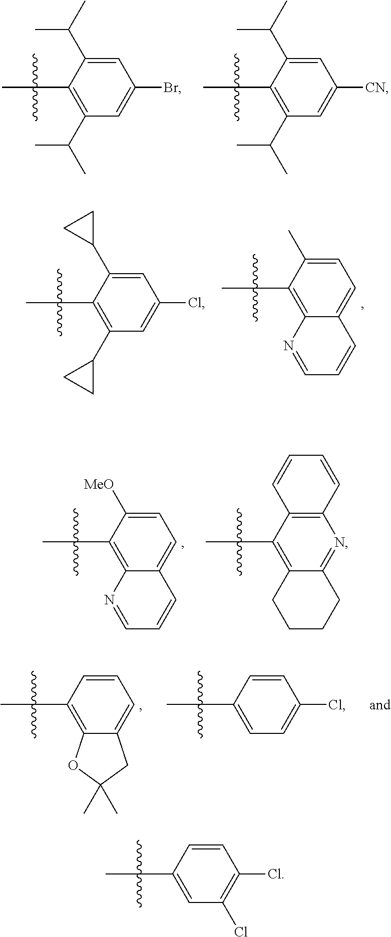

or a pharmaceutically acceptable salt thereof.

In another aspect, provided herein is a compound of Formula AA

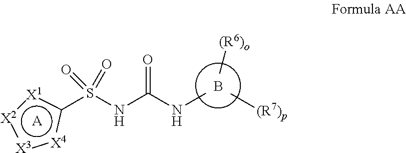

wherein the compound of Formula AA is selected from

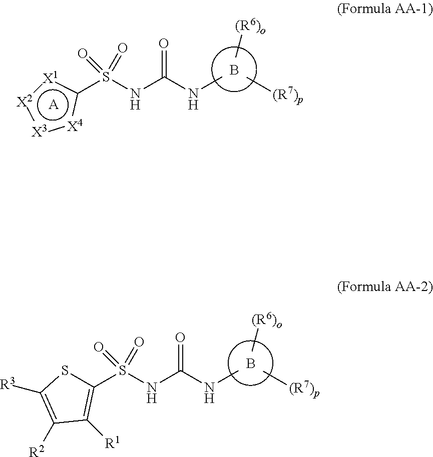

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
when X⁴ is —X⁵—X⁶, then: X¹ is N or CR¹, X² is N or CR², and X³ is N or CR³;
when X⁴ is other than —X⁵—X⁶—, then

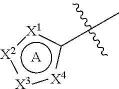

comprises at least one of CR¹, CR², CR³, and CR⁴;
when X⁴ is —X⁵—X⁶—, then

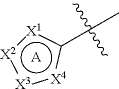

comprises at least two of CR¹, CR², CR³, CR⁵, and CR⁶;
wherein when X¹ is S, X⁴ is other than CR⁴;
wherein when X⁴ is S, X¹ is other than CR¹;
wherein when the compound of Formula AA is a compound of Formula AA-1, from two to four of R¹, R², R³, and R⁴ are present or from two to five of R¹, R², R³, R⁵, and R⁶ are present; and
wherein at least two of the two to four R¹, R², R³, and R⁴ or at least of two to five R¹, R², R³, R⁵, and R⁶ are on adjacent atoms;
wherein when the compound of Formula AA is a compound of Formula AA-1, any two adjacent R¹, R², R³, and R⁴ or any two adjacent R¹, R², R³, R⁵, and R⁶ are taken together with the atoms connecting them to form a ring; and wherein when the compound of Formula AA is a compound of Formula AA-2, any two adjacent R¹, R², and R³ are taken together with the atoms connecting them to form a ring; wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heteroaryl ring is optionally substituted with one or more R²⁰;

R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹; or at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, —OC(O)NHC₆₋₁₀ aryl optionally substituted with one or more R²¹, and CONR⁸R⁹;

or at least one pair of R²⁰ on the adjacent atoms, taken together with the atoms connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;

R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
or at least one pair of R²¹ on adjacent atoms, taken together with the atoms connecting them, independently forms a C₄-C₁₂ cycloalkyl ring or a 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;
wherein the remaining R¹, R², R³, and R⁴, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;

R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;

R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
wherein when the compound is a compound of Formula AA-1,

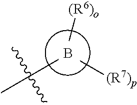

is selected from the group consisting of:

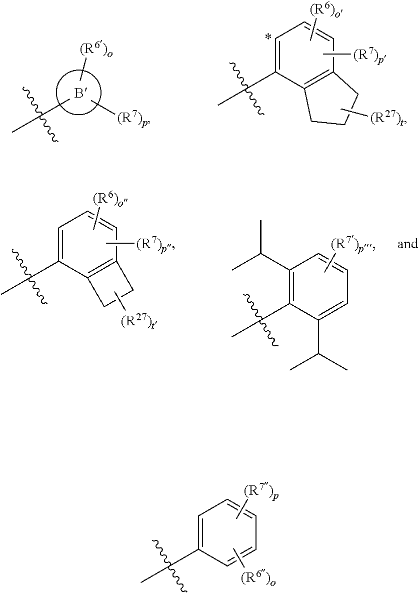

wherein * denotes that the ring position the * is closest to is unsubstituted;
B is a 5-10-membered heteroaryl or C₆-C₁₀ aryl ring;
B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members; 4-pyrimidinyl; 5-pyrimidinyl; 6-pyrimidinyl; pyridazinyl; pyrazinyl; 1,2,3-triazinyl; 1,2,4-triazinyl; tetrazinyl; imidazolyl; pyrazolyl; 1,2,3-triazolyl; tetrazolyl; or C₇-C₁₀ aryl;
o=1 or 2;
p=0, 1, 2, or 3;
o′=0 or 1;
p′=0 or 1;
o″=0 or 1;
p″=0, 1, or 2;
p′″=1, 2, or 3;
t is 0, 1, 2, 3, 4, 5, or 6;
t′ is 0, 1, 2, 3, or 4;
R⁶ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{6′} at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R⁷ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{7′}, at each occurrence, is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, I, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵,
each occurrence of R^{6″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
each occurrence of R^{7″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷;
or at least one pair of R^{6′} and R⁷ on adjacent atoms, taken together with the atoms connecting them, independently form at least one C₄-C₈ (e.g, C₄-C₅ or C₇-C₈) cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷;
or at least one pair of R^{6″} and R^{7″} on adjacent atoms, taken together with the atoms connecting them, independently form at least one C₄ or C₆-C₈ carbocyclic ring, wherein the carbocyclic ring is optionally independently substituted with one or more R²⁷;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
R²⁷, at each occurrence, is independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
or a pharmaceutically acceptable salt thereof.

In another aspect, provided herein is a compound of Formula AA

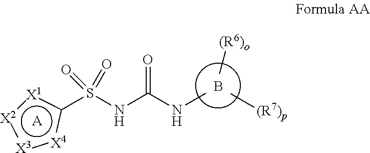

wherein the compound of Formula AA is selected from

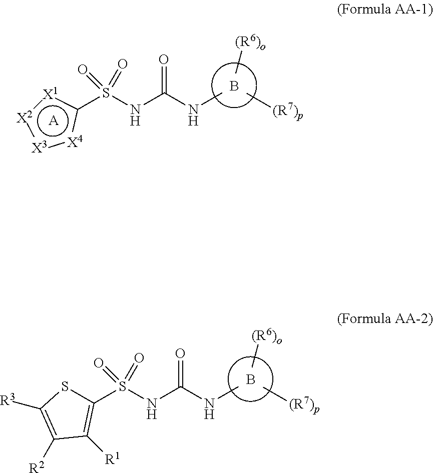

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
when X⁴ is —X⁵—X⁶, then: X¹ is N or CR¹, X² is N or CR², and X³ is N or CR³;
when X⁴ is other than —X⁵—X⁶—, then

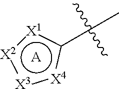

comprises at least one of CR¹, CR², CR³, and CR⁴;
when X⁴ is —X⁵—X⁶—, then

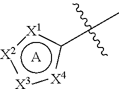

comprises at least two of CR¹, CR², CR³, CR⁵, and CR⁶;
wherein when X¹ is S, X⁴ is other than CR⁴;
wherein when X⁴ is S, X¹ is other than CR¹;
wherein when the compound of Formula AA is a compound of Formula AA-1, from two to four of R¹, R², R³, and R⁴ are present or from two to five of R¹, R², R³, R⁵, and R⁶ are present; and
wherein at least two of the two to four R¹, R², R³, and R⁴ or at least of two to five R¹, R², R³, R⁵, and R⁶ are on adjacent atoms;
wherein when the compound of Formula AA is a compound of Formula AA-1, any two adjacent R¹, R², R³, and R⁴ or any two adjacent R¹, R², R³, R⁵, and R⁶ are taken together with the atoms connecting them to form a ring; and wherein when the compound of Formula AA is a compound of Formula AA-2, any two adjacent R¹, R², and R³ are taken together with the atoms connecting them to form a ring; wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heteroaryl ring is optionally substituted with one or more R²⁰;

R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹;

or at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;

or at least one pair of R²⁰ on the adjacent atoms, taken together with the atoms connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;

R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
wherein the remaining R¹, R², R³, and R⁴, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;

R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;

R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
wherein when the compound is a compound of Formula AA-1,

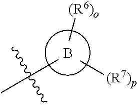

is selected from the group consisting of:

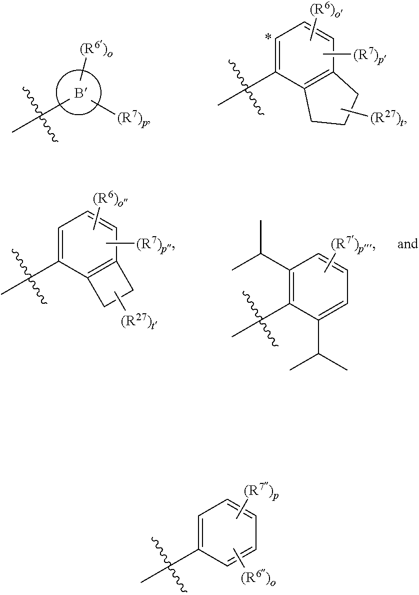

wherein * denotes that the ring position the * is closest to is unsubstituted;
B is a 5-10-membered heteroaryl or C₆-C₁₀ aryl ring;
B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members; 4-pyrimidinyl; 5-pyrimidinyl; 6-pyrimidinyl; pyridazinyl; pyrazinyl; 1,2,3-triazinyl; 1,2,4-triazinyl; tetrazinyl; imidazolyl; pyrazolyl; 1,2,3-triazolyl; tetrazolyl; or C₇-C₁₀ aryl;
o=1 or 2;
p=0, 1, 2, or 3;
o′=0 or 1;
p′=0 or 1;
o″=0 or 1;
p″=0, 1, or 2;
p′″=1, 2, or 3;
t is 0, 1, 2, 3, 4, 5, or 6;
t′ is 0, 1, 2, 3, or 4;
R⁶ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{6′} at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R⁷ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{7′}, at each occurrence, is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, I, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵, each occurrence of R^{6″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵; each occurrence of R^{7″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷;
or at least one pair of R^{6′} and R⁷ on adjacent atoms, taken together with the atoms connecting them, independently form at least one C₄-C₈ (e.g, C₄-C₅ or C₇-C₈) cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷;
or at least one pair of R^{6″} and R^{7″} on adjacent atoms, taken together with the atoms connecting them, independently form at least one C₄ or C₆-C₈ carbocyclic ring, wherein the carbocyclic ring is optionally independently substituted with one or more R²⁷;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
R²⁷, at each occurrence, is independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
or a pharmaceutically acceptable salt thereof.

In another aspect, provided herein is a compound of Formula AA

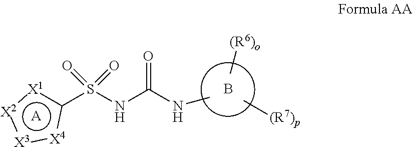

wherein the compound of Formula AA is selected from

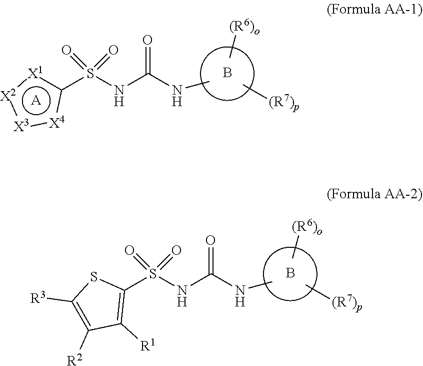

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
wherein when X⁴ is —X⁵—X⁶, then:
X¹ is N or CR¹;
X² is N or CR²;
X³ is N or CR³;
when X⁴ is other than —X⁵—X⁶—, then

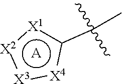

comprises at least one of CR¹, CR², CR³, and CR⁴;
when X⁴ is —X⁵—X⁶—, then

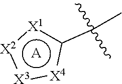

comprises at least two of CR¹, CR², CR³, CR⁵, and CR⁶; wherein when X¹ is S, X⁴ is other than CR⁴;
wherein when X⁴ is S, X¹ is other than CR¹;
wherein when the compound of Formula AA is a compound of Formula AA-1, from two to four of R¹, R², R³, and R⁴ are present or from two to five of R¹, R², R³, R⁵, and R⁶ are present; and
wherein at least two of the two to four R¹, R², R³, and R⁴ or at least of two to five R¹, R², R³, R⁵, and R⁶ are on adjacent atoms;
wherein when the compound of Formula AA is a compound of Formula AA-1, any two adjacent R¹, R², R³, and R⁴ or any two adjacent R¹, R², R³, R⁵, and R⁶ are taken together with the atoms connecting them to form a ring; and wherein when the compound of Formula AA is a compound of Formula AA-2, any two adjacent R¹, R², and R³ are taken together with the atoms connecting them to form a ring; wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heteroaryl ring is optionally substituted with one or more R²⁰;

R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹;

or at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;

R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
wherein the remaining R¹, R², R³, and R⁴, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;
R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;
R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
wherein when the compound is a compound of Formula AA-1,

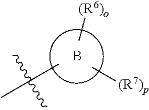

is selected from the group consisting of:

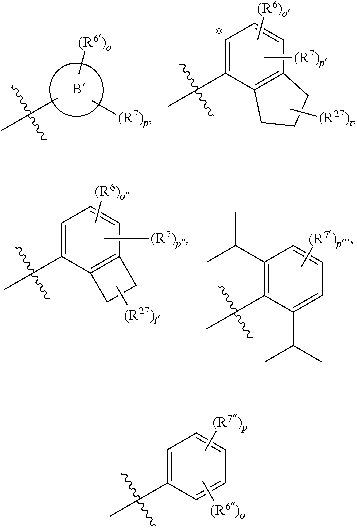

wherein * denotes that the ring position the * is closest to is unsubstituted;
B is a 5-10-membered heteroaryl or C₆-C₁₀ aryl;
B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members; 5-pyrimidinyl; 6-pyrimidinyl; pyridazinyl; pyrazinyl; 1,2,3-triazinyl; 1,2,4-triazinyl; tetrazinyl; imidazolyl; pyrazolyl; 1,2,3-triazolyl; tetrazolyl; or C₇-C₁₀ aryl;
o=1 or 2;
p=0, 1, 2, or 3;
o′=0 or 1;
p′=0 or 1;
o″=0 or 1;
p″=0, 1, or 2;
p′″=1, 2, or 3;
t is 0, 1, 2, 3, 4, 5, or 6;
t′ is 0, 1, 2, 3, or 4;
R⁶ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{6′} at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R⁷ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{7′}, at each occurrence, is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, I, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵,
each occurrence of R^{6″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
wherein when

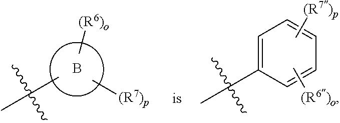

at least one R is ortho to the bond connecting the B ring to the NH(CO) group of Formula AA;
each occurrence of R^{7″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷;
or at least one pair of R^{6′} and R⁷ on adjacent atoms, taken together with the atoms connecting them, independently form at least one C₄-C₈ cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷;
or at least one pair of R^{6″} and R^{7″} on adjacent atoms, taken together with the atoms connecting them, independently form at least one C₄ or C₆-C₈ carbocyclic ring, wherein the carbocyclic ring is optionally independently substituted with one or more R²⁷;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
R²⁷, at each occurrence, is independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
or a pharmaceutically acceptable salt thereof.

### The Formula AA

In some embodiments, the compound of Formula AA is a compound of Formula AA-1:

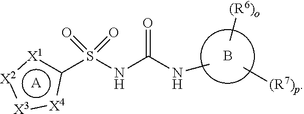

In some embodiments, the compound of Formula AA is a compound of Formula AA-2:

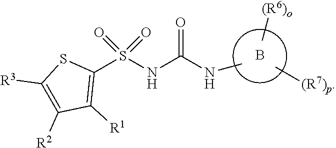

In some embodiments the variables shown in the formulae herein are as follows:

### The Moiety

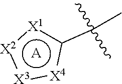

In some embodiments,

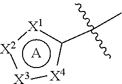

is selected from the group consisting of:

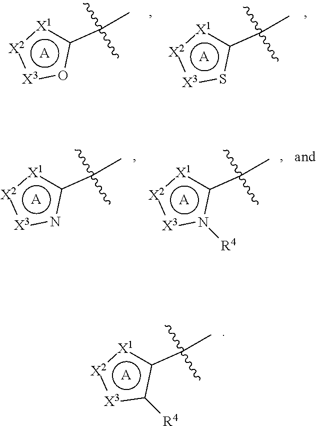

In some embodiments,

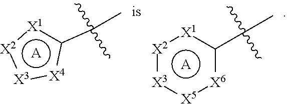

In some embodiments,

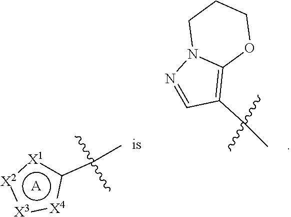

In some embodiments,

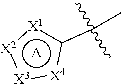

is selected from the group consisting of:

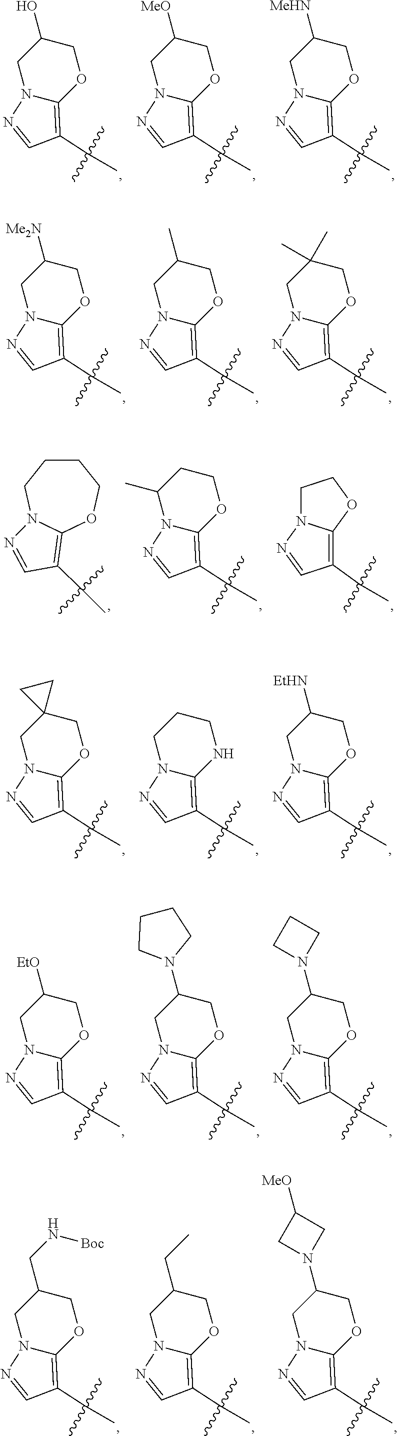

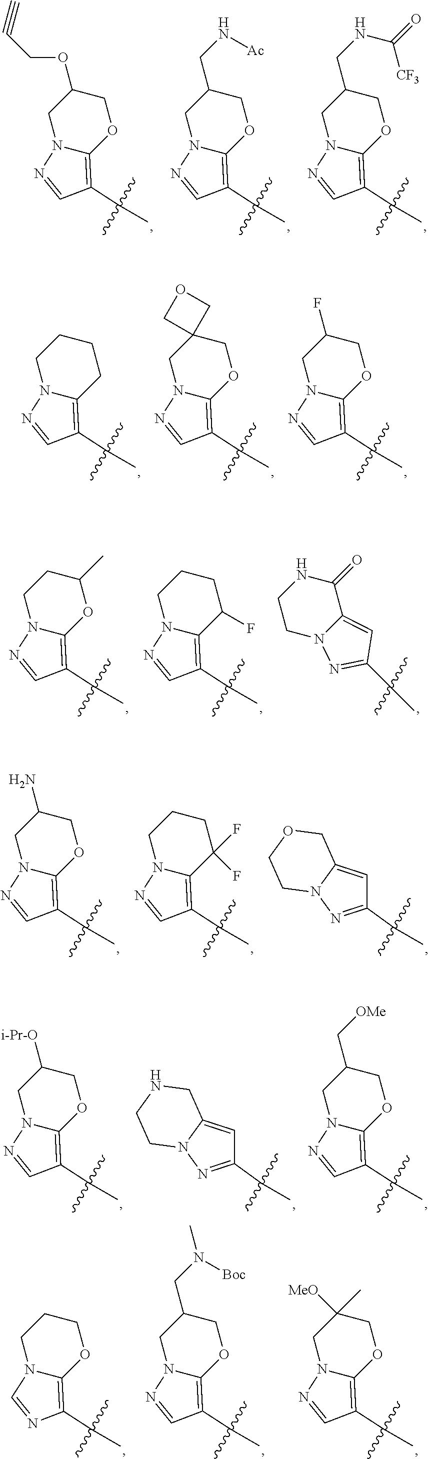

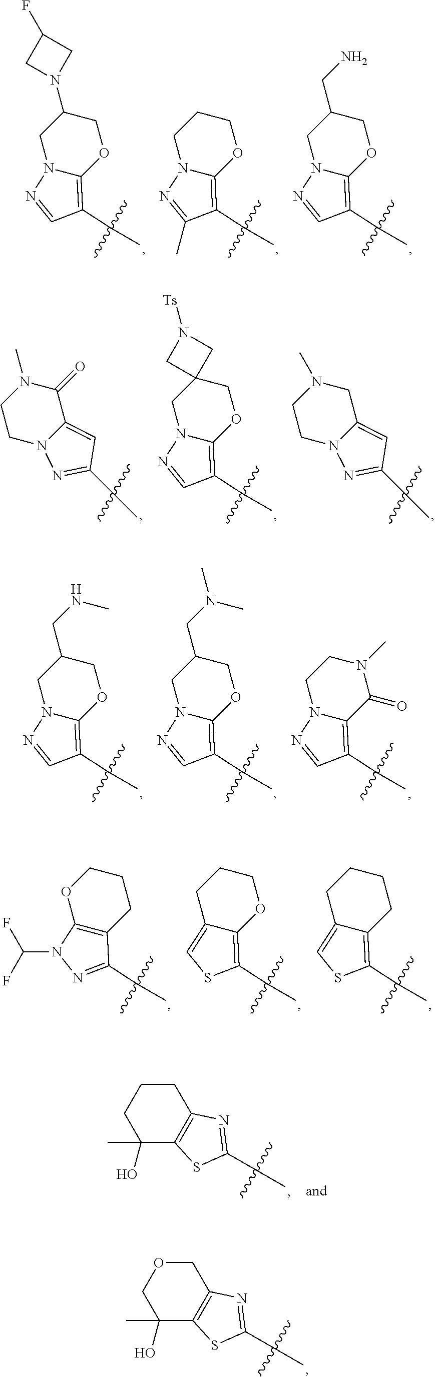

In some embodiments,

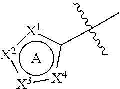

selected from the group consisting of:

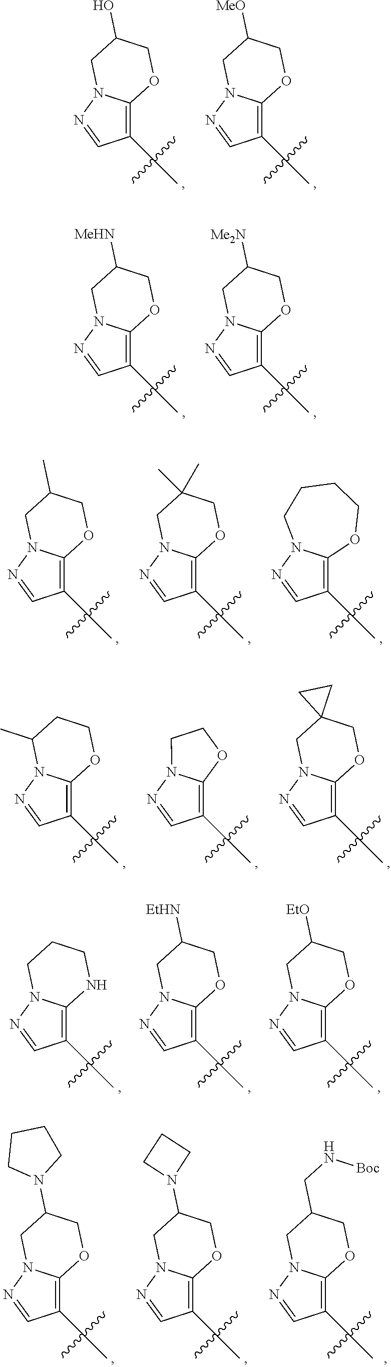

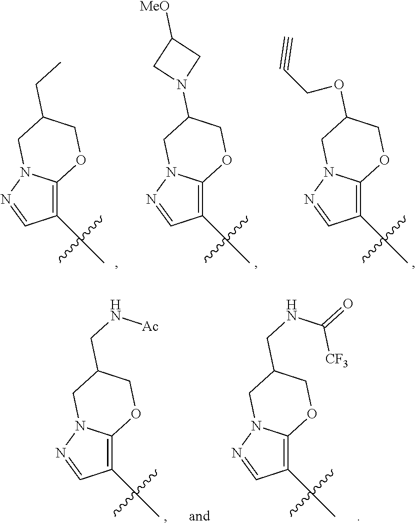 [SMILES: *c1cnn2c1OCC(CC)C2 ; *c1cnn2c1OCC(N1CC(OC)C1)C2 ; *c1cnn2c1OCC(OCC#C)C2 ; *c1cnn2c1OCC(CNC(C)=O)C2 ; *c1cnn2c1OCC(CNC(=O)C(F)(F)F)C2]

In some embodiments,

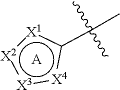

is selected from the group consisting of:

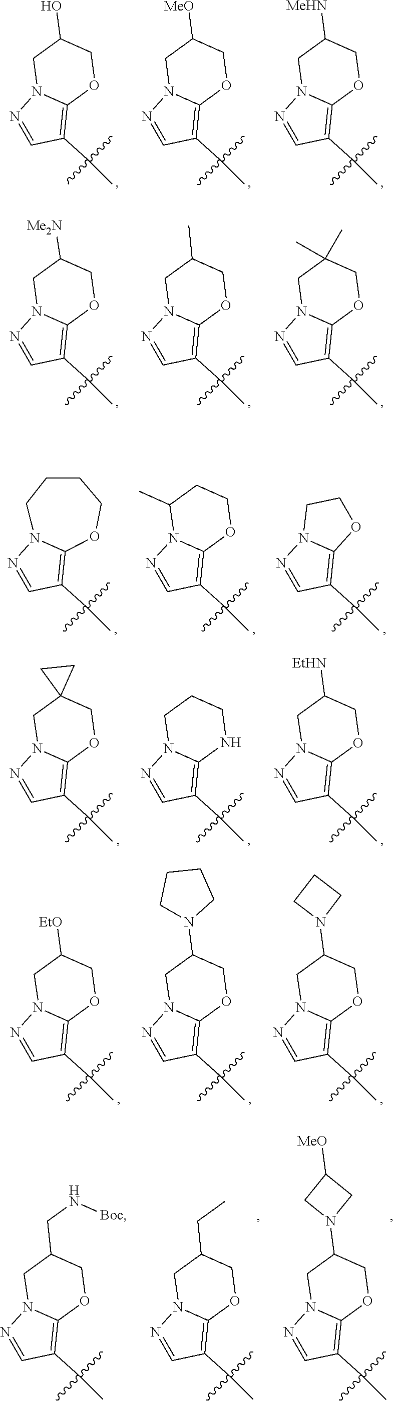

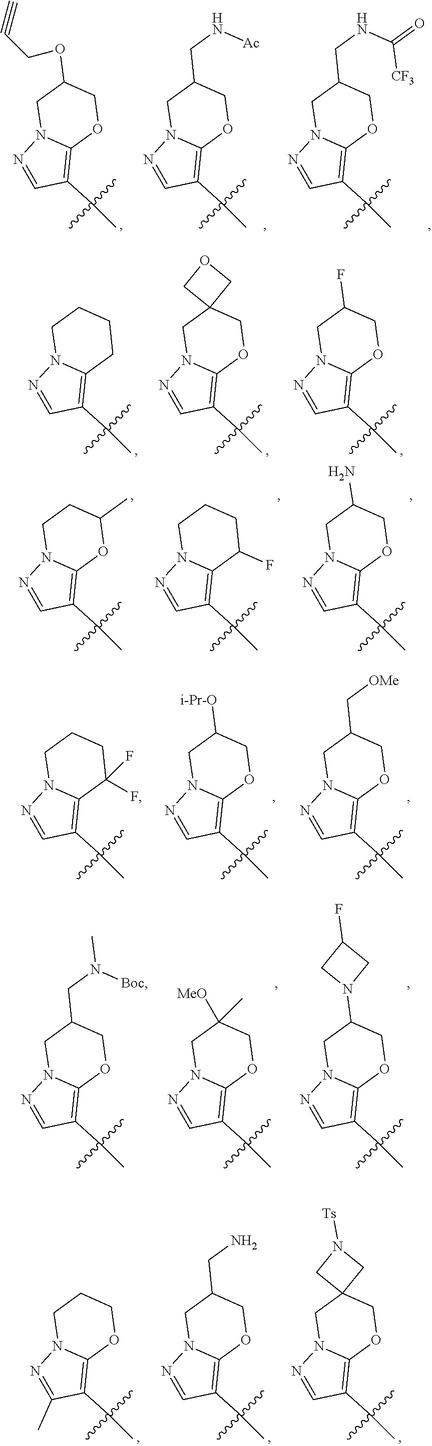

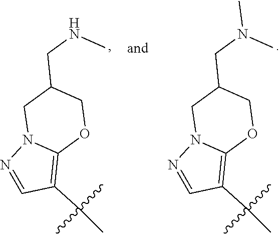 [SMILES: *c1cnn2c1OCC(CNC)C2 ; *c1cnn2c1OCC(CN(C)C)C2]

In some embodiments,

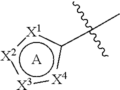

is selected from the group consisting of:

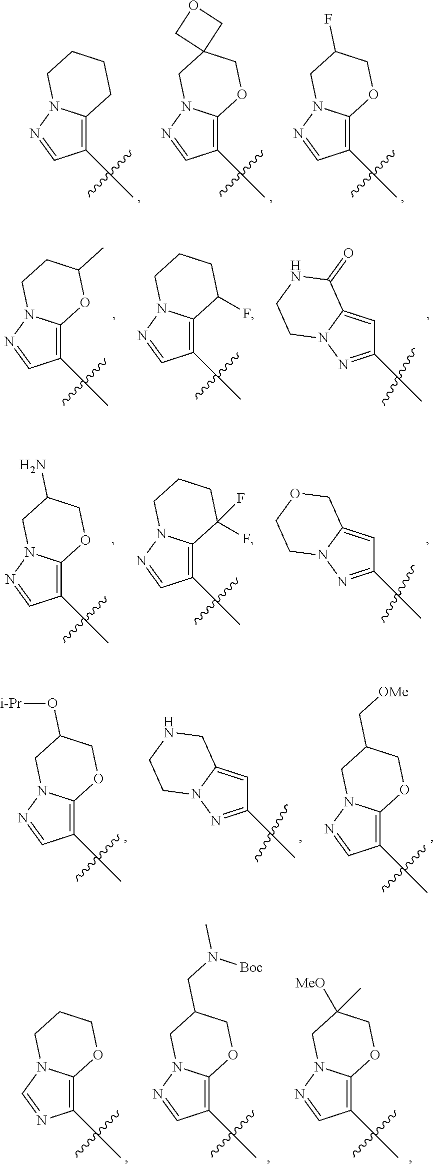

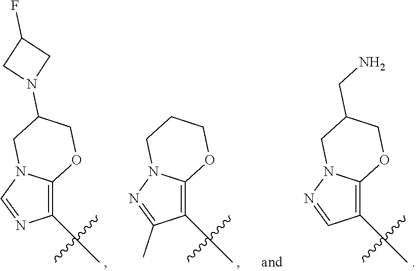 [SMILES: *c1ncn2c1OCC(N1CC(F)C1)C2 ; *c1c(C)nn2c1OCCC2 ; *c1cnn2c1OCC(CN)C2]

In some embodiments,

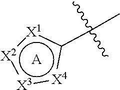

is selected from the group consisting of:

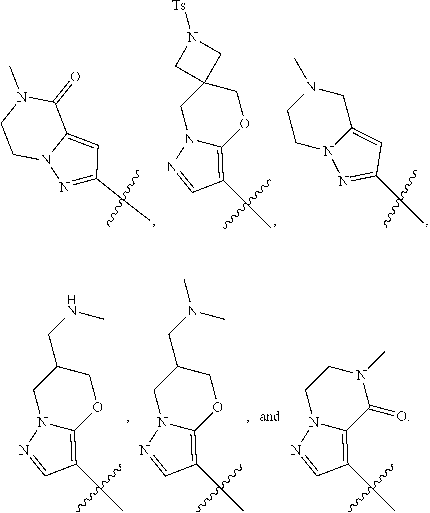

In some embodiments,

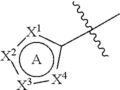

is selected from the group consisting of:

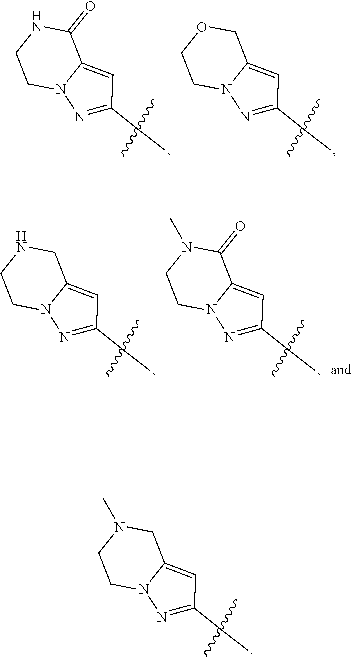 [SMILES: *c1cc2n(n1)CCNC2=O ; *c1cc2n(n1)CCOC2 ; *c1cc2n(n1)CCNC2 ; *c1cc2n(n1)CCN(C)C2=O ; *c1cc2n(n1)CCN(C)C2]

In some embodiments,

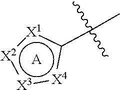

is selected from the group consisting of:

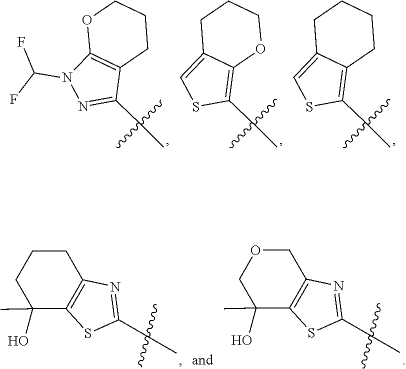

In some embodiments,

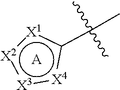

is selected from the group consisting of:

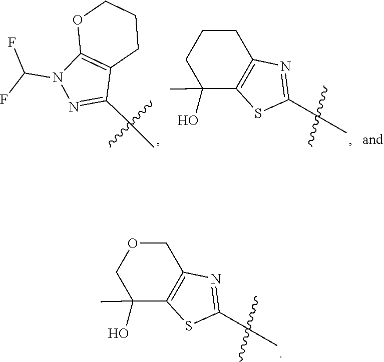 [SMILES: *c1nn(C(F)F)c2c1CCCO2 ; *c1nc2c(s1)C(C)(O)CCC2 ; *c1nc2c(s1)C(C)(O)COC2]

In some embodiments,

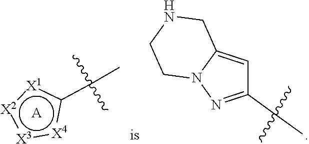

In some embodiments,

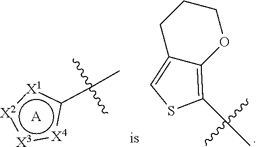

In some embodiments,

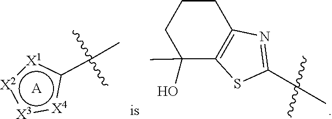

In some embodiments,

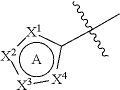

is selected from the group consisting of:

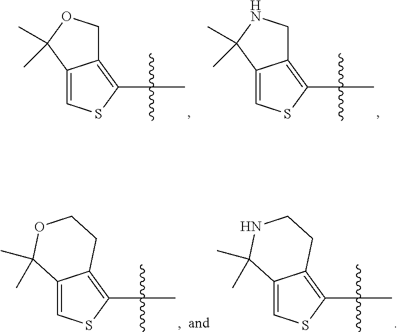 [SMILES: *c1scc2c1COC2(C)C ; *c1scc2c1CNC2(C)C ; *c1scc2c1CCOC2(C)C ; *c1scc2c1CCNC2(C)C]

In some embodiments,

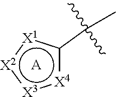

is other than

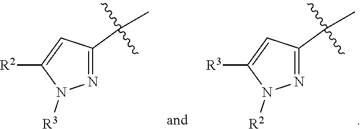

For example

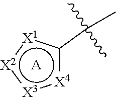

is other than

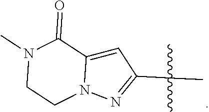 [SMILES: *c1cc2n(n1)CCN(C)C2=O]

### The Moiety

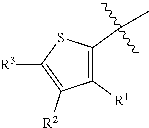

### R²

In some embodiments,

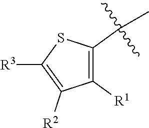

is selected from the group consisting of:

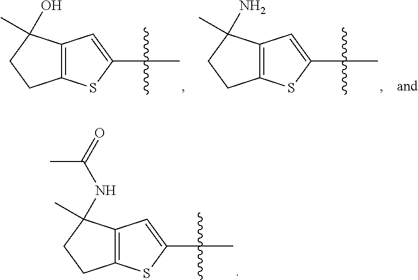

In some embodiments,

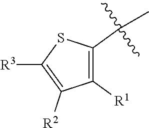

is selected from the group consisting of:

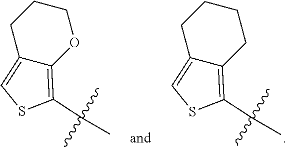 [SMILES: *c1scc2c1OCCC2 ; *c1scc2c1CCCC2]

In some embodiments,

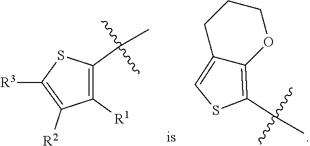

The Variables X X², X³, X⁴, X⁵, and X⁶
In some embodiments, when X⁴ is other than —X⁵—X⁶—, each of X¹, X², X³, X⁴, X⁵, and X⁶ is other than N.
In some embodiments, when (i) X⁴ is other than —X⁵—X⁶—, and (ii) each of X¹, X², X³, X⁴, X⁵, and X⁶ is other than N, then at least two of the two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰.

In some embodiments, when X⁴ is other than —X⁵—X⁶—, then

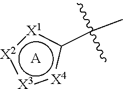

comprises at least one of CR¹, CR², CR³, and CR⁴.
In some embodiments, when X⁴ is —X⁵—X⁶—, then

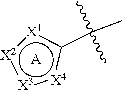

comprises at least two of CR¹, CR², CR³, CR⁵, and CR⁶.
In some embodiments, X¹ and X⁴ are each other than O.
In some embodiments, X¹ and X⁴ are each other than N.
In some embodiments, when one of X¹ and X⁴ is N; the other of X¹ and X⁴ is other than O.
In some embodiments, when one of X¹ and X⁴ is O; the other of X¹ and X⁴ is other than N.
In certain embodiments, when: X⁴ is other than —X⁵—X⁶—; one of X¹ and X⁴ is selected from the group consisting of O, S, and NH; the other of X¹ and X⁴ is N, CR¹, or CR⁴; X² is CR²; X³ is CR³; and R² and R³ are taken together with the atoms connecting them to form a ring; then B is other than phenyl or 4-pyridyl.
In some embodiments, X⁴ is —X⁵—X⁶—.
In some embodiments, X⁵ is N.
In some embodiments, X⁵ is CR⁵.
In some embodiments, X⁶ is NR⁵.
In some embodiments, X⁶ is CR⁵.
In some embodiments, X¹ is O.
In some embodiments, X¹ is S.
In some embodiments, X¹ is N.
In some embodiments, X¹ is NR¹.
In some embodiments, X¹ is CR¹.
In some embodiments, X² is O.
In some embodiments, X² is S.
In some embodiments, X² is N.
In some embodiments, X² is NR².
In some embodiments, X² is CR².
In some embodiments, X³ is O.
In some embodiments, X³ is S.
In some embodiments, X³ is N.
In some embodiments, X³ is NR³.
In some embodiments, X³ is CR³.
In some embodiments, X⁴ is O.
In some embodiments, X⁴ is S.
In some embodiments, X⁴ is N.
In some embodiments, X⁴ is NR⁴.
In some embodiments, X⁴ is CR⁴.
In certain embodiments, X¹ is CR¹; and X² is NR².
In some embodiments, X³ is N; and X⁴ is CR⁴.
In certain embodiments, X¹ is CR¹; X² is NR²; X³ is N; and X⁴ is CR⁴ (i.e., the

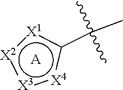

moiety is

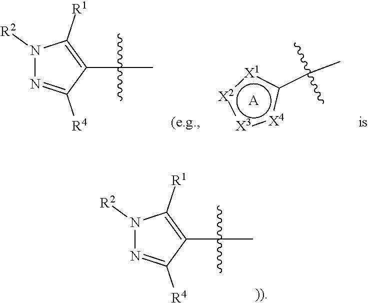

In some embodiments, X¹ is N.
In certain embodiments, X² is NR²; and X³ is CR³.
In certain embodiments, X³ is CR³; and X⁴ is CR⁴.
In certain embodiments, X¹ is N; X² is NR²; X³ is CR³; and X⁴ is CR⁴ (i.e., the

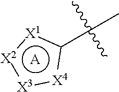

moiety is

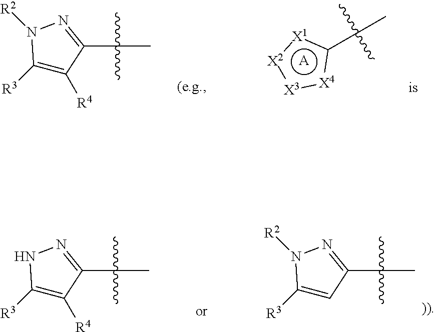

In certain embodiments, X³ is NR³; and X⁴ is CR⁴.
In certain embodiments, X¹ is N; X² is CR²; X³ is NR³; and X⁴ is CR⁴ (i.e., the

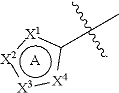

moiety is

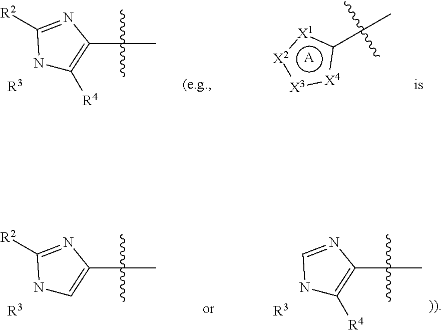

In certain embodiments, X¹ is N; X² is CR²; X³ is CR³; and X⁴ is NR⁴ (i.e., the

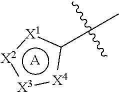

moiety is

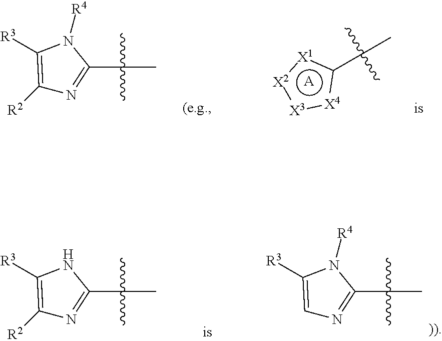

In some embodiments, X¹ is S; and X² is CR².
In certain embodiments, X² is CR²; and X³ is CR³.
In certain embodiments, X³ is CR³; and X⁴ is CR⁴.
In certain embodiments, X¹ is S; X² is CR²; X³ is CR³; and X⁴ is CR⁴ (i.e., the

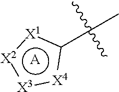

moiety is

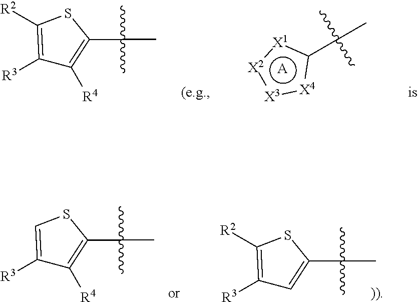

In some embodiments, X¹ is CR¹; and X² is S.
In some embodiments X³ is CR³; and X⁴ is CR⁴.
In certain embodiments, X¹ is CR¹; X² is S; X³ is CR³; and X⁴ is CR⁴ (i.e., the

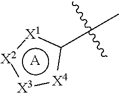

moiety is

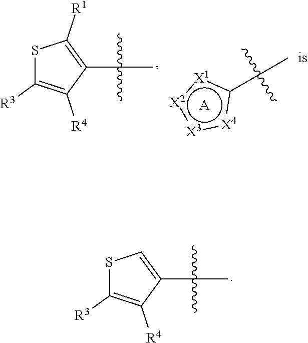

In certain embodiments, X² is CR²; and X³ is CR³.
In certain embodiments, X¹ is S; X² is CR²; X³ is CR³; and X⁴ is N (i.e., the

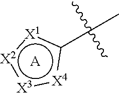

moiety is

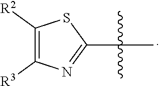

In certain embodiments, X² is CR²; and X³ is CR³.
In certain embodiments, X¹ is O; X² is CR²; X³ is CR³; and X⁴ is N (i.e., the

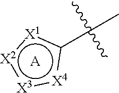

moiety is

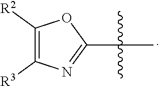

In certain embodiments, X² is CR²; and X³ is CR³.
In certain embodiments, X³ is CR³; and X⁴ is CR⁴.
In certain embodiments, X¹ is O; X² is CR²; X³ is CR³; and X⁴ is CR⁴ (i.e., the

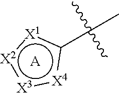

moiety is

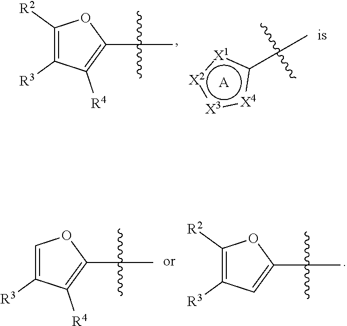

In some embodiments, X¹ is CR¹; and X² is O.
In some embodiments X³ is CR³; and X⁴ is CR⁴.
In certain embodiments, X¹ is CR¹; X² is O; X³ is CR³; and X⁴ is CR⁴ (i.e., the

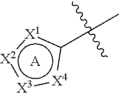

moiety is

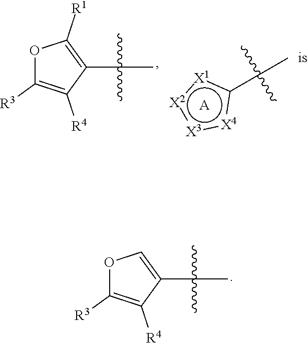

In certain embodiments, X² is CR²; and X³ is CR³.
In certain embodiments, X³ is CR³; and X⁴ is CR⁴.
In certain embodiments, X¹ is NR¹; X² is CR²; X³ is CR³; and X⁴ is CR⁴ (i.e., the

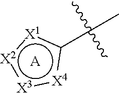

moiety is

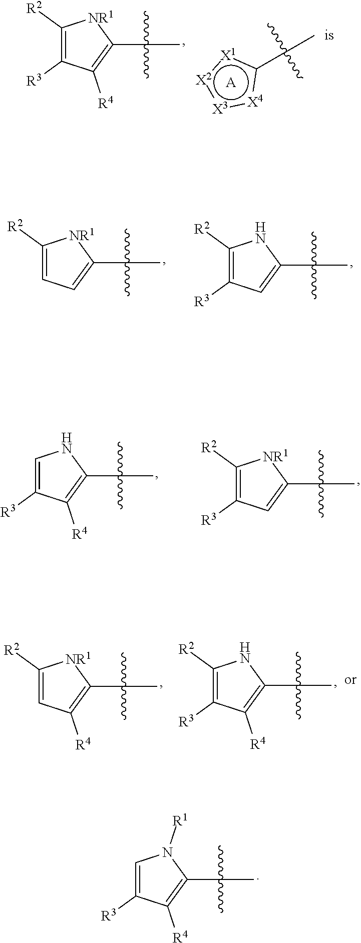

In some embodiments, X¹ is CR¹; and X² is NR².
In some embodiments X is CR³; and X⁴ is CR⁴.
In certain embodiments, X¹ is CR¹; X² is NR²; X³ is CR³; and X⁴ is CR⁴ (i.e., the

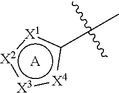

moiety is

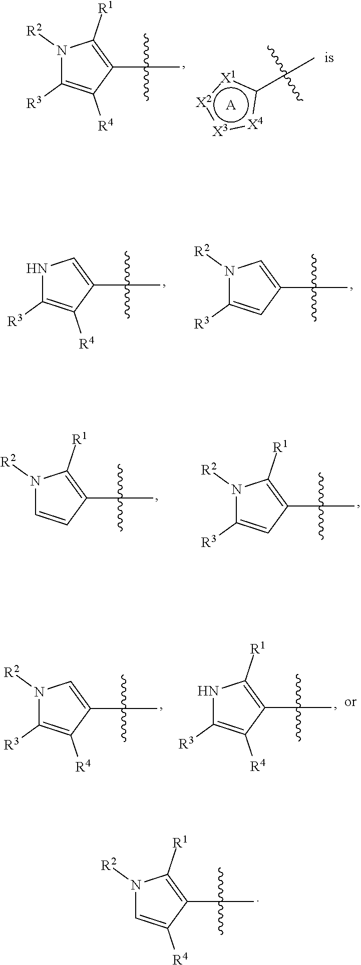

In some embodiments, X¹ is N; and X² is CR².
In some embodiments X³ is NR³; and X⁴ is N.
In certain embodiments, X¹ is N; X² is CR²; X³ is NR³; and X⁴ is N (i.e., the

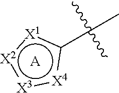

moiety is

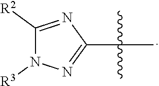

In some embodiments, X¹ is NR¹; and X² is CR².
In some embodiments X³ is N; and X⁴ is N.
In certain embodiments, X¹ is NR¹; X² is CR²; X³ is N; and X⁴ is N (i.e., the

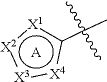

moiety is

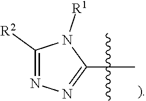

In some embodiments, X¹ is N; and X² is N.
In some embodiments X³ is NR³; and X⁴ is CR⁴.
In certain embodiments, X¹ is N; X² is N; X³ is NR³; and X⁴ is CR⁴ (i.e., the

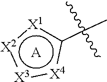

moiety is

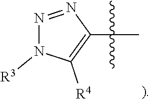

In certain embodiments, X¹ is N; X² is S; X³ is CR³; and X⁴ is CR⁴ (i.e., the

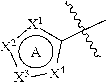

moiety is

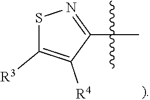

In certain embodiments, X¹ is S; X² is N; X³ is CR³; and X⁴ is CR⁴ (i.e., the

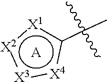

moiety is

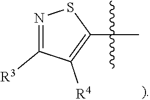

In certain embodiments, X¹ is NH; X² is N; X³ is CR³; and X⁴ is CR⁴ (i.e., the

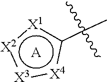

moiety is

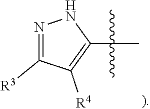

In certain embodiments, X¹ is N; X² is O; X³ is CR³; and X⁴ is CR⁴ (i.e., the

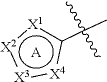

moiety is

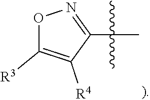

In certain embodiments. X¹ is O; X² is N; X³ is CR³; and X⁴ is CR⁴ (i.e., the

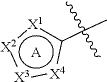

moiety is

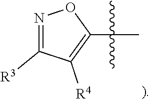

In certain embodiments, X¹ is CR¹; X² is CR²; X³ is CR³; and X⁴ is —X⁵—X⁶—; X⁵ is CR⁵; and X⁶ is CR⁶ (i.e., the

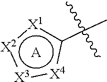

moiety is

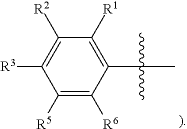

In certain embodiments, X¹ is CR¹; X² is CR²; X³ is CR³; X⁴ is —X⁵—X⁶—; X⁵ is CR⁵; and X⁶ is N (i.e., the

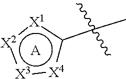

moiety is

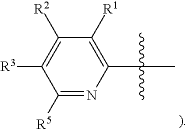

In certain embodiments, X¹ is N; X² is CR²; X³ is CR³; X⁴ is —X⁵—X⁶—; X⁵ is CR⁵; and X⁶ is CR⁶ (i.e., the

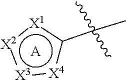

moiety is

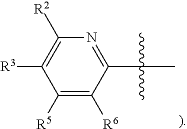

In certain embodiments, X¹ is CR¹; X² is N; X³ is CR³; X⁴ is —X⁵—X⁶—; X⁵ is CR⁵; and X⁶ is CR⁶
(i.e., the

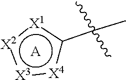

moiety is

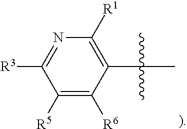

In certain embodiments, X¹ is N; X² is CR²; X³ is CR³; X⁴ is —X⁵—X⁶—; X⁵ is CR⁵; and X⁶ is N
(i.e., the

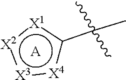

moiety is

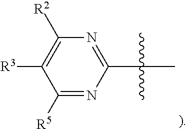

In certain embodiments, X¹ is N; X² is N; X³ is CR³; X⁴ is —X⁵—X⁶—; X⁵ is CR⁵; and X⁶ is CR⁶
(i.e., the

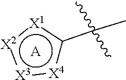

moiety is

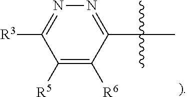

In certain embodiments, X¹ is CR¹; X² is N; X³ is CR³; X⁴ is —X⁵—X⁶—; X⁵ is CR⁵; and X⁶ is N (i.e., the

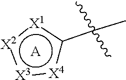

moiety is

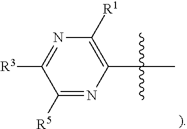

In certain embodiments, X¹ is N; X² is N; X³ is CR³; X⁴ is —X⁵—X⁶—; X⁵ is CR⁵; and X⁶ is N (i.e., the

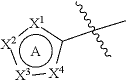

moiety is

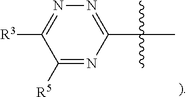

In certain embodiments, X¹ is CR¹; X² is CR²; X³ is N; X⁴ is —X⁵—X⁶—; X⁵ is CR⁵; and X⁶ is CR⁶ (i.e., the

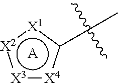

moiety is

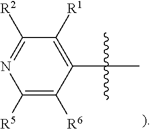

In certain embodiments, X¹ is CR¹; X² is N; X³ is N; X⁴ is —X⁵—X⁶—; X⁵ is CR⁵; and X⁶ is CR⁶ (i.e., the

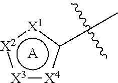

moiety is

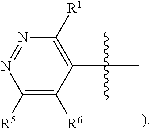

In certain embodiments, X¹ is N; X² is CR²; X³ is N; X⁴ is —X⁵—X⁶—; X⁵ is CR⁵; and X⁶ is CR⁶ (i.e., the

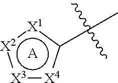

moiety is

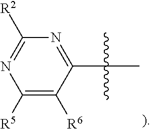

In certain embodiments, X¹ is CH; X² is CH; X³ is CR³; and X⁴ is —X⁵—X⁶—; X⁵ is CR⁵; and X⁶ is CH (i.e., the

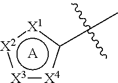

moiety is

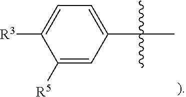

In certain embodiments, X¹ is CH; X² is CH; X³ is CR³; X⁴ is —X⁵—X⁶—; X⁵ is CR⁵; and X⁶ is N (i.e., the

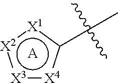

moiety is

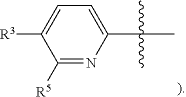

In certain embodiments, X¹ is N; X² is CH; X³ is CR³; X⁴ is —X⁵—X⁶—; X⁵ is CR⁵; and X⁶ is CH (i.e., the

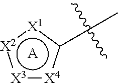

moiety is

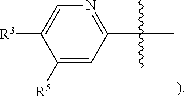

In certain embodiments, X¹ is CH; X² is N; X³ is CR³; X⁴ is —X⁵—X⁶—; X⁵ is CR⁵; and X⁶ is CH (i.e., the

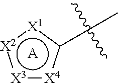

moiety is

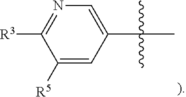

In certain embodiments, X¹ is N; X² is CH; X³ is CR³; X⁴ is —X⁵—X⁶—; X⁵ is CR⁵; and X⁶ is N (i.e., the

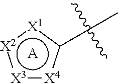

moiety is

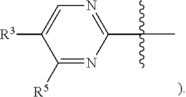

In certain embodiments, X¹ is N; X² is N; X³ is CR³; X⁴ is —X⁵—X⁶—; X⁵ is CR⁵; and X⁶ is CH (i.e., the

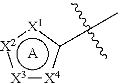

moiety is

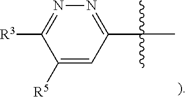

In certain embodiments, X¹ is CH; X² is N; X³ is CR³; X⁴ is —X⁵—X⁶—; X⁵ is CR⁵; and X⁶ is N (i.e., the

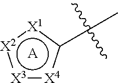

moiety is

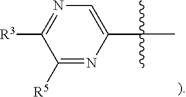

In certain embodiments, X¹ is N; X² is N; X³ is CR³; X⁴ is —X⁵—X⁶—; X⁵ is CR⁵; and X⁶ is N (i.e., the

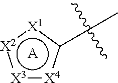

moiety is

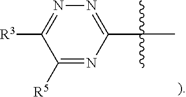

In certain embodiments, X¹ is CH; X² is CH; X³ is CH; X⁴ is —X⁵—X⁶—; X⁵ is CR⁵; and X⁶ is CR⁶ (i.e., the

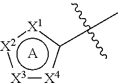

moiety is

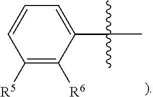

In certain embodiments, X¹ is N; X² is CH; X³ is CH; X⁴ is —X⁵—X⁶—; X⁵ is CR⁵; and X⁶ is CR⁶ (i.e., the

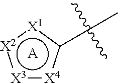

moiety is

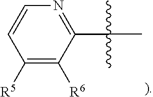

In certain embodiments, X¹ is CH; X² is N; X³ is CH; X⁴ is —X⁵—X⁶—; X⁵ is CR⁵; and X⁶ is CR⁶ (i.e., the

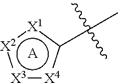

moiety is

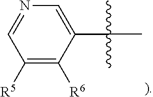

In certain embodiments, X¹ is CH; X² is CH; X³ is N; X⁴ is —X⁵—X⁶—; X⁵ is CR⁵; and X⁶ is CR⁶ (i.e., the

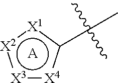

moiety is

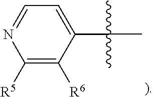

In certain embodiments, X¹ is N; X² is N; X³ is CH; X⁴ is —X⁵—X⁶—; X⁵ is CR⁵; and X⁶ is CR⁶ (i.e., the

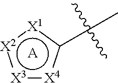

moiety is

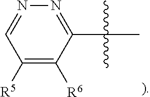

In certain embodiments, X¹ is CH; X² is N; X³ is N; X⁴ is —X⁵—X⁶—; X⁵ is CR⁵; and X⁶ is CR⁶ (i.e., the

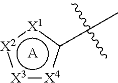

moiety is

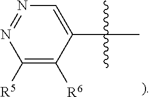

In certain embodiments, X¹ is N; X² is CH; X³ is N; X⁴ is —X⁵—X⁶—; X⁵ is CR⁵; and X⁶ is CR⁶ (i.e., the

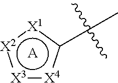

moiety is

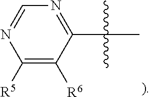

The Variables X¹, X², X³, and X⁴ when Formula AA is Formula AA-1
In some embodiments, when X⁴ is other than —X⁵—X⁶—, each of X¹, X², X³, X⁴, X⁵, and X⁶ is other than N.
In some embodiments, when (i) X⁴ is other than —X⁵—X⁶—, and (ii) each of X¹, X², X³, X⁴, X⁵, and X⁶ is other than N, then at least two of the two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰.

In some embodiments, X¹ and X⁴ are each other than O.
In some embodiments, X¹ and X⁴ are each other than N.
In some embodiments, when one of X¹ and X⁴ is N; the other of X¹ and X⁴ is other than O.
In some embodiments, when one of X¹ and X⁴ is O; the other of X¹ and X⁴ is other than N.
In some embodiments, X⁴ is other than —X⁵—X⁶—.
In some embodiments, X¹ is O.
In some embodiments, X¹ is S.
In some embodiments, X¹ is N.
In some embodiments, X¹ is NR¹.
In some embodiments, X¹ is CR¹.
In some embodiments, X² is O.
In some embodiments, X² is S.
In some embodiments, X² is N.
In some embodiments, X² is NR².
In some embodiments, X² is CR².
In some embodiments, X³ is O.
In some embodiments, X³ is S.
In some embodiments, X³ is N.
In some embodiments, X³ is NR³.
In some embodiments, X³ is CR³.
In some embodiments, X⁴ is O.
In some embodiments, X⁴ is S.
In some embodiments, X⁴ is N.
In some embodiments, X⁴ is NR⁴.
In some embodiments, X⁴ is CR⁴.
In certain embodiments, X¹ is CR¹; and X² is NR².
In certain embodiments, X¹ is CR¹; X² is NR²; X³ is N; and X⁴ is CR⁴ (i.e., the

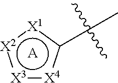

moiety is

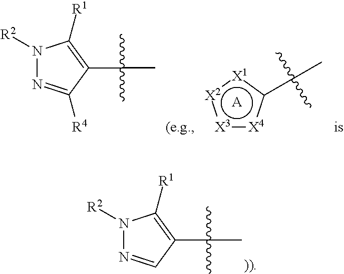

In certain embodiments, X³ is CR³; and X⁴ is CR⁴.
In certain embodiments, X¹ is N; X² is NR²; X³ is CR³; and X⁴ is CR⁴.
In certain embodiments, X¹ is N; X² is NR²; X³ is CR³; and X⁴ is CR⁴ (i.e., the

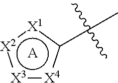

moiety is

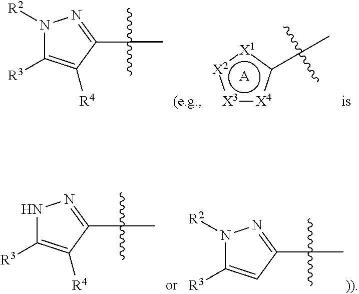

In certain embodiments, X³ is NR³; and X⁴ is CR⁴.
In certain embodiments, X¹ is N; X² is CR²; X³ is NR³; and X⁴ is CR⁴ (i.e., the

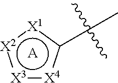

moiety is

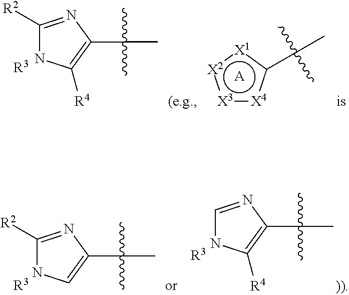

In certain embodiments, X¹ is N; X² is CR²; X³ is CR³; and X⁴ is NR⁴ (i.e., the

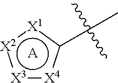

moiety is

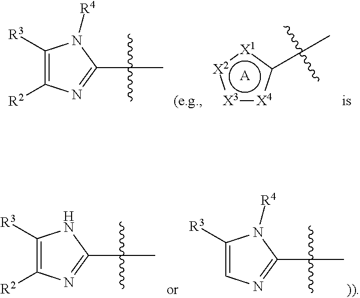

In certain embodiments, X² is CR²; and X³ is CR³.
In certain embodiments, X¹ is S; X² is CR²; X³ is CR³; and X⁴ is N (i.e., the

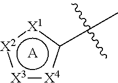

moiety is

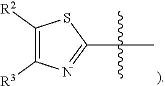

In some embodiments, X¹ is CR¹; and X² is S.
In some embodiments X³ is CR³; and X⁴ is CR⁴.
In certain embodiments, X¹ is CR¹; X² is S; X³ is CR³; and X⁴ is CR⁴.
In certain embodiments, X¹ is CR¹; X² is S; X³ is CR³; and X⁴ is CR⁴ (i.e., the

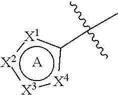

moiety is

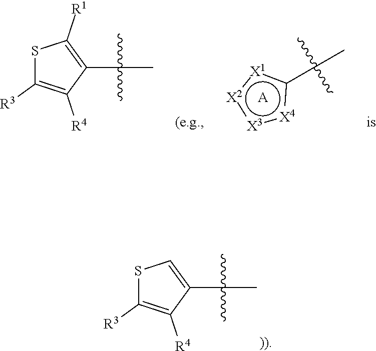

In certain embodiments, X² is CR²; and X³ is CR³.
In certain embodiments, X¹ is O; X² is CR²; X³ is CR³; and X⁴ is N (i.e., the

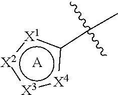

moiety is

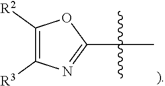

In certain embodiments, X² is CR²; and X³ is CR³.
In certain embodiments, X³ is CR³; and X⁴ is CR⁴.
In certain embodiments, X¹ is O; X² is CR²; X³ is CR³; and X⁴ is CR⁴ (i.e., the

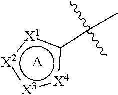

moiety is

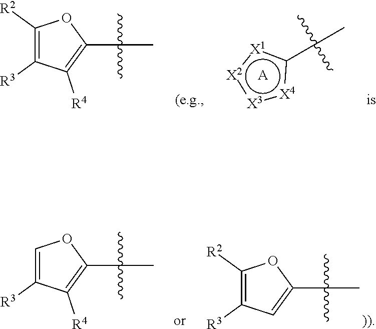

In some embodiments, X¹ is CR¹; and X² is O.
In some embodiments X³ is CR³; and X⁴ is CR⁴.
In certain embodiments, X¹ is CR¹; X² is O; X³ is CR³; and X⁴ is CR⁴ (i.e., the

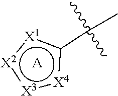

moiety is

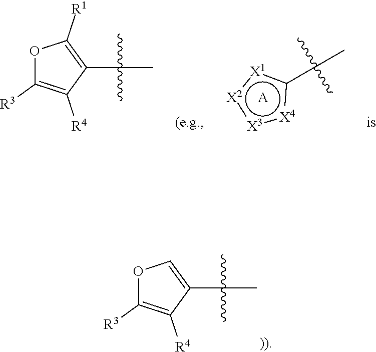

In certain embodiments, X² is CR²; and X³ is CR³.
In certain embodiments, X³ is CR³; and X⁴ is CR⁴.
In certain embodiments, X¹ is NR¹; X² is CR²; X³ is CR³; and X⁴ is CR⁴ (i.e., the

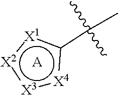

moiety is

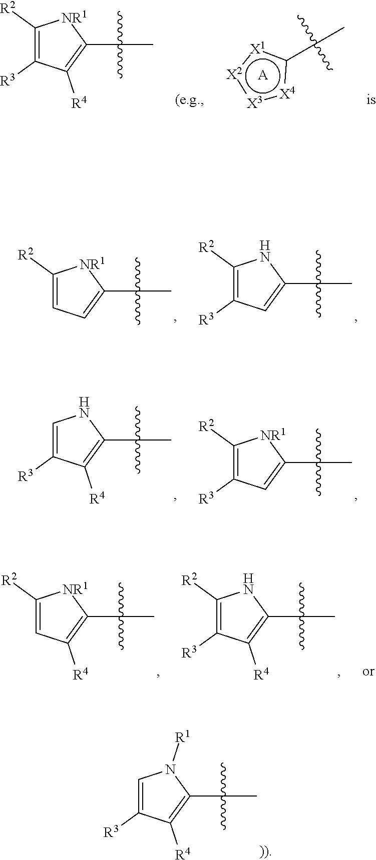

In some embodiments, X¹ is CR¹; and X² is NR².
In some embodiments X³ is CR³; and X⁴ is CR⁴.
In certain embodiments, X¹ is CR¹; X² is NR²; X³ is CR³; and X⁴ is CR⁴ (i.e., the

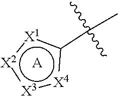

moiety is

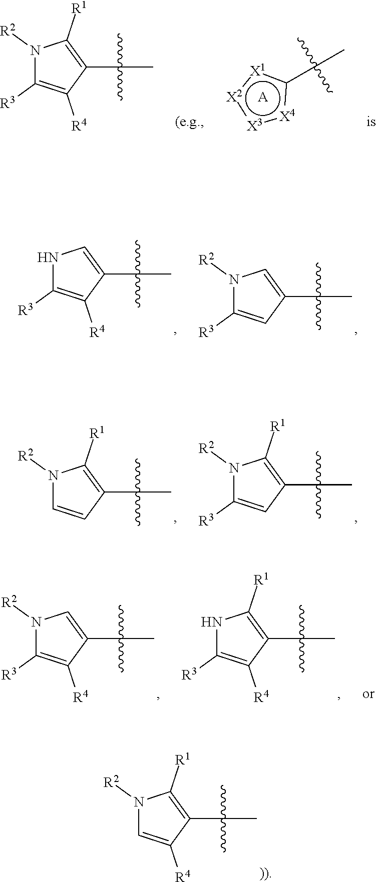

In some embodiments, X¹ is N; and X² is CR².
In some embodiments X³ is NR³; and X⁴ is N.
In certain embodiments, X¹ is N; X² is CR²; X³ is NR³; and X⁴ is N (i.e., the

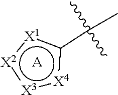

moiety is

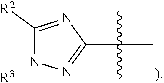

In some embodiments, X¹ is NR¹; and X² is CR².
In some embodiments X³ is N; and X⁴ is N.
In certain embodiments, X¹ is NR¹; X² is CR²; X³ is N; and X⁴ is N (i.e., the

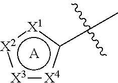

moiety is

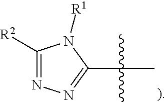

In some embodiments, X¹ is N; and X² is N.
In some embodiments X³ is NR³; and X⁴ is CR⁴.
In certain embodiments, X¹ is N; X² is N; X³ is NR³; and X⁴ is CR⁴ (i.e., the

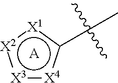

moiety is

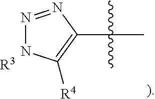

In certain embodiments, X¹ is N; X² is S; X³ is CR³; and X⁴ is CR⁴ (i.e., the

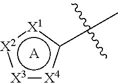

moiety is

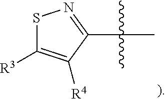

In certain embodiments, X¹ is S; X² is N; X³ is CR³; and X⁴ is CR⁴ (i.e., the

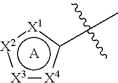

moiety is

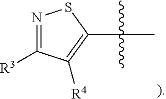

In certain embodiments, X¹ is NH; X² is N; X³ is CR³; and X⁴ is CR⁴ (i.e., the

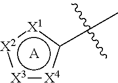

moiety is

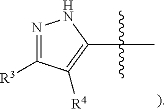

In certain embodiments, X¹ is N; X² is O; X³ is CR³; and X⁴ is CR⁴ (i.e., the

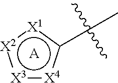

moiety is

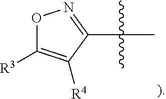

In certain embodiments, X¹ is O; X² is N; X³ is CR³; and X⁴ is CR⁴ (i.e., the

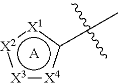

moiety is

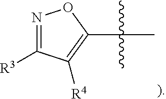

In certain embodiments, X¹ is CR¹; X² is CR²; X³ is CR³; and X⁴ is —X⁵—X⁶—; X⁵ is CR⁵; and X⁶ is CR⁶ (i.e., the

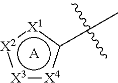

moiety is

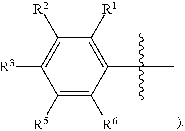

In certain embodiments, X¹ is CR¹; X² is CR²; X³ is CR³; X⁴ is —X⁵—X⁶—; X⁵ is CR⁵; and X⁶ is N (i.e., the

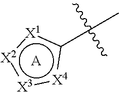

moiety is

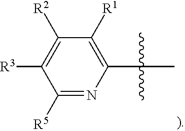

In certain embodiments, X¹ is N; X² is CR²; X³ is CR³; X⁴ is —X⁵—X⁶—; X⁵ is CR⁵; and X⁶ is CR⁶ (i.e., the

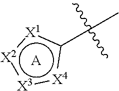

moiety is

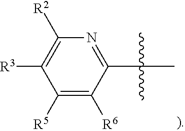

In certain embodiments, X¹ is CR¹; X² is N; X³ is CR³; X⁴ is —X⁵—X⁶—; X⁵ is CR⁵; and X⁶ is CR⁶ (i.e., the

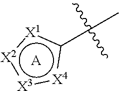

moiety is

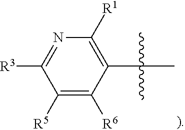

In certain embodiments, X¹ is N; X² is CR²; X³ is CR³; X⁴ is —X⁵—X⁶—; X⁵ is CR⁵; and X⁶ is N (i.e., the

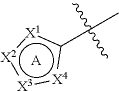

moiety is

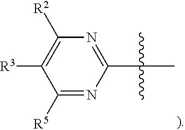

In certain embodiments, X¹ is N; X² is N; X³ is CR³; X⁴ is —X⁵—X⁶—; X⁵ is CR⁵; and X⁶ is CR^{6 (i.e., the}

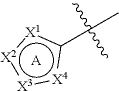

moiety is

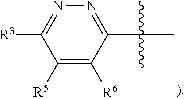

In certain embodiments, X¹ is CR¹; X² is N; X³ is CR³; X⁴ is —X⁵—X⁶—; X⁵ is CR⁵; and X⁶ is N (i.e., the

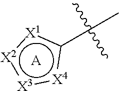

moiety is

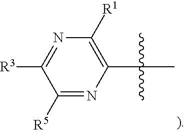

In certain embodiments, X¹ is N; X² is N; X³ is CR³; X⁴ is —X⁵—X⁶—; X⁵ is CR⁵; and X⁶ is N (i.e., the

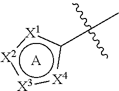

moiety is

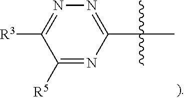

In certain embodiments, X¹ is CR¹; X² is CR²; X³ is N; X⁴ is —X⁵—X⁶—; X⁵ is CR⁵; and X⁶ is CR⁶ (i.e., the

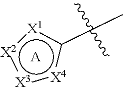

moiety is

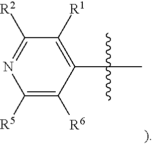

In certain embodiments, X¹ is CR¹; X² is N; X³ is N; X⁴ is —X⁵—X⁶—; X⁵ is CR⁵; and X⁶ is CR⁶ (i.e., the

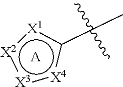

moiety is

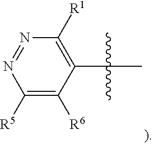

In certain embodiments, X¹ is N; X² is CR²; X³ is N; X⁴ is —X⁵—X⁶—; X⁵ is CR⁵; and X⁶ is CR⁶ (i.e., the

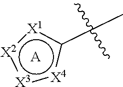

moiety is

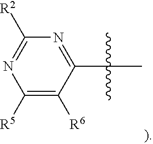

In certain embodiments. X¹ is CH; X² is CH; X³ is CR³; X⁴ is —X⁵—X⁶—; X⁵ is CR⁵; and X⁶ is CH (i.e., the

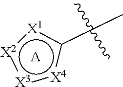

moiety is

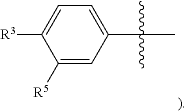

In certain embodiments, X¹ is CH; X² is CH; X³ is CR³; X⁴ is —X⁵—X⁶—; X⁵ is CR⁵; and X⁶ is N (i.e., the

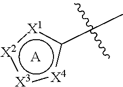

moiety is

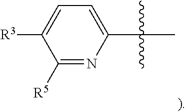

In certain embodiments, X¹ is N; X² is CH; X³ is CR³; X⁴ is —X⁵—X⁶—; X⁵ is CR⁵; and X⁶ is CH (i.e., the

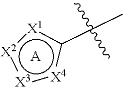

moiety is

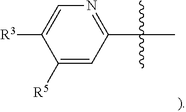

In certain embodiments, X¹ is CH; X² is N; X³ is CR³; X⁴ is —X⁵—X⁶—; X⁵ is CR⁵; and X⁶ is CH (i.e., the

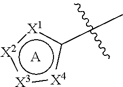

moiety is

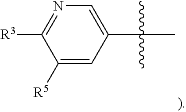

In certain embodiments, X¹ is N; X² is CH; X³ is CR³; X⁴ is —X⁵—X⁶—; X⁵ is CR⁵; and X⁶ is N (i.e., the

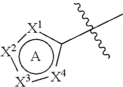

moiety is

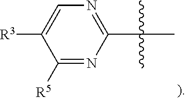

In certain embodiments, X¹ is N; X² is N; X³ is CR³; X⁴ is —X⁵—X⁶—; X⁵ is CR⁵; and X⁶ is CH (i.e., the

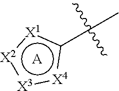

moiety is

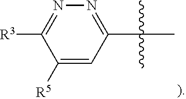

In certain embodiments, X¹ is CH; X² is N; X³ is CR³; X⁴ is —X⁵—X⁶—; X⁵ is CR⁵; and X⁶ is N (i.e., the

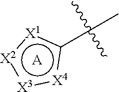

moiety is

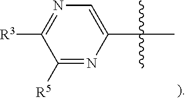

In certain embodiments, X¹ is N; X² is N; X³ is CR³; X⁴ is —X⁵—X⁶—; X⁵ is CR⁵; and X⁶ is N (i.e., the

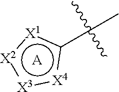

moiety is

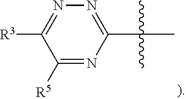

In certain embodiments, X¹ is CH; X² is CH; X³ is CH; X⁴ is —X⁵—X⁶—; X⁵ is CR⁵; and X⁶ is CR⁶ (i.e., the

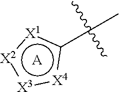

moiety is

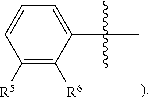

In certain embodiments, X¹ is N; X² is CH; X³ is CH; X⁴ is —X⁵—X⁶—; X⁵ is CR⁵; and X⁶ is CR⁶ (i.e., the

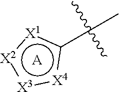

moiety is

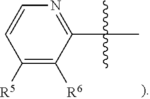

In certain embodiments, X¹ is CH; X² is N; X³ is CH; X⁴ is —X⁵—X⁶—; X⁵ is CR⁵; and X⁶ is CR⁶ (i.e., the

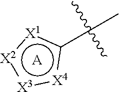

moiety is

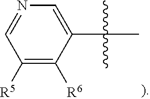

In certain embodiments. X¹ is CH; X² is CH; X³ is N; X⁴ is —X⁵—X⁶—; X⁵ is CR⁵; and X⁶ is CR⁶ (i.e., the

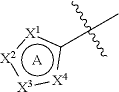

moiety is

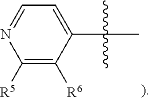

In certain embodiments, X¹ is N; X² is N; X³ is CH; X⁴ is —X⁵—X⁶—; X⁵ is CR⁵; and X⁶ is CR⁶ (i.e., the

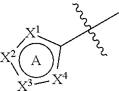

moiety is

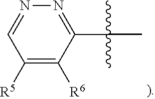

In certain embodiments, X¹ is CH; X² is N; X³ is N; X⁴ is —X⁵—X⁶—; X⁵ is CR⁵; and X⁶ is CR⁶ (i.e., the

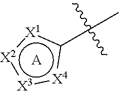

moiety is

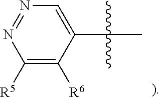

In certain embodiments, X¹ is N; X² is CH; X³ is N; X⁴ is —X⁵—X⁶—; X⁵ is CR⁵; and X⁶ is CR⁶ (i.e., the

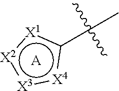

moiety is

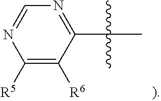

In some embodiments,

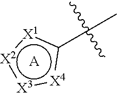

is selected from

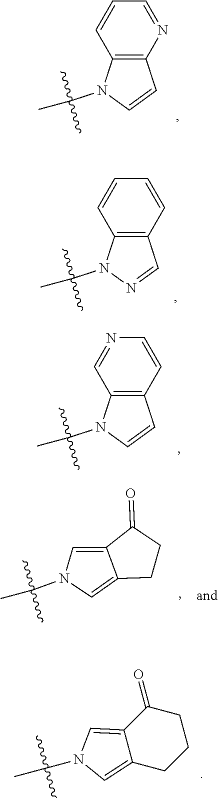 [SMILES: *n1ccc2ncccc21 ; *n1ncc2ccccc21 ; *n1ccc2ccncc21 ; *n1cc2c(c1)C(=O)CC2 ; *n1cc2c(c1)C(=O)CCC2]

The Groups R¹, R², R³, R⁴, R⁵, and R⁶

For purposes of clarification, when a monocyclic or bicyclic 5- to-12-membered heterocycloalkyl ring or 5-10 membered heteroaryl ring contains 1-3 heteroatoms and/or heteroatomic groups independently selected from O, N, NH, NR¹³, and S, wherein the heteroatom or heteroatomic group is cumulative with the values selected for X¹, X², X³, and X⁴, the 1-3 cumulative heteroatom or heteroatomic group are a set that does not include the values selected

for X¹, X², X³, and X⁴. For example, in

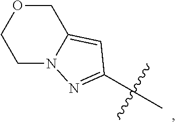 [SMILES: *c1cc2n(n1)CCOC2]

there is 1 heteroatom (i.e., oxygen) cumulative with the values selected for X¹, X², X³, and X⁴.

For purposes of clarification, definitions along the lines of “wherein at least two of the two to four R¹, R², R³, and R⁴ or at least two of the two to five R¹, R², R³, R⁵, and R⁶ are on adjacent atoms, and taken together with the atoms connecting them, independently form a monocyclic or bicyclic C₄-C₁₂ cycloalkyl ring optionally substituted with one or more R²⁰ are intended to encompass structures, in which one of the adjacent ring atoms is N (i.e., a bridgehead N) e.g.,

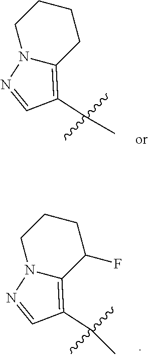 [SMILES: *c1cnn2c1CCCC2 ; *c1cnn2c1C(F)CCC2]

For purposes of clarification, definitions along the lines of “wherein at least two of the two to four R¹, R², R³, and R⁴ or at least two of the two to five R¹, R², R³, R⁵, and R⁶ are on adjacent atoms, and taken together with the atoms connecting them, independently form a monocyclic or bicyclic 5- to-12-membered heterocycloalkyl ring containing 1-3 heteroatoms and/or heteroatomic groups independently selected from O, N, NH, NR¹³, and S, are intended to encompass structures, in which i) one or neither of the adjacent ring atoms is N; and ii) at least one other ring atom in addition to said adjacent ring atoms is a heteroatom, e.g.,

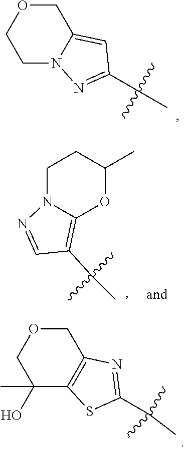

It is understood that in embodiments of any applicable formulae herein that when any two adjacent substituents selected from R¹, R², R³, R⁴, R⁵, and R⁶ are taken together with the atoms connecting them to form a cycloalkyl ring, the atoms connecting the two substituents that are taken together are selected from C and N.

For example, when R¹ and R² on adjacent atoms are taken together with the atoms connecting them to form a monocyclic or bicyclic C₄-C₁₂ cycloalkyl ring optionally substituted with one or more R²⁰, then X¹ is selected from CR¹ and XR¹, and X² is selected from CR² and NR².

For example, when R² and R³ on adjacent atoms are taken together with the atoms connecting them to form a monocyclic or bicyclic C₄-C₁₂ cycloalkyl ring optionally substituted with one or more R²⁰, then X² is selected from CR² and XR², and X³ is selected from CR³ and NR³.

For example, when R³ and R⁴ on adjacent atoms are taken together with the atoms connecting them to form a monocyclic or bicyclic C₄-C₁₂ cycloalkyl ring optionally substituted with one or more R²⁰, then X³ is selected from CR³ and XR³, and X⁴ is selected from CR⁴ and NR⁴.

For example, when R⁴ and R⁵ on adjacent atoms are taken together with the atoms connecting them to form a monocyclic or bicyclic C₄-C₁₂ cycloalkyl ring optionally substituted with one or more R²⁰, then X⁴ is selected from CR⁴ and XR⁴, and X⁵ is selected from CR⁵ and NR⁵.

For example, when R⁵ and R⁶ on adjacent atoms are taken together with the atoms connecting them to form a monocyclic or bicyclic C₄-C₁₂ cycloalkyl ring optionally substituted with one or more R²⁰, then X⁵ is selected from CR⁵ and XR⁵, and X⁶ is selected from CR⁶ and NR⁶.

In some embodiments of Formula AA and Formula AA-1 herein, X⁴ is —X⁵—X⁶—; from two to five of R¹, R², R³, R⁵, and R⁶ are present; and wherein at least of two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments of Formula AA and Formula AA-1 herein, X⁴ is —X⁵—X⁶—; from two to five of R¹, R², R³, R⁵, and R⁶ are present; and wherein at least two of the two to five R¹, R², R³, R⁵, and R⁶ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰, and

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰. In some embodiments of Formula AA, Formula AA-1, and Formula AA-2 herein, X⁴ is other than —X⁵—X⁶—; from two to four of R¹, R², R³, and R⁴ are present; and wherein at least two of the two to four R¹, R², R³, and R⁴ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, wherein the heteroaryl ring is optionally substituted with one or more R²⁰;

In some embodiments of Formula AA, Formula AA-1, and Formula AA-2 herein, X⁴ is other than —X⁵—X⁶—; from two to four of R¹, R², R³, and R⁴ are present; and wherein at least two of the two to four R¹, R², R³, and R⁴ are on adjacent atoms, and taken together with the atoms connecting them, independently form a ring selected from the group consisting of:

(i) a monocyclic or bicyclic C₄-C₁₂ cycloalkyl ring optionally substituted with one or more R²⁰, and

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰.

In some embodiments, R¹ and R² are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹ and X², and wherein the heteroaryl ring is optionally substituted with one or more R²⁰.

In some embodiments, R¹ and R² are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰.

In some embodiments, R¹ and R² are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S (e.g., O and NH), wherein the heteroatom or heteroatomic group is cumulative with the values selected for X¹ and X², and wherein the heterocycloalkyl ring is optionally substituted with one or more R²⁰.

In some embodiments, R¹ and R², taken together with the atoms connecting them, form a monocyclic or bicyclic C₄-C₁₂ cycloalkyl ring optionally substituted with one or more R²⁰.

In some embodiments, R¹ and R², taken together with the atoms connecting them, form a monocyclic C₅-C₆ cycloalkyl ring optionally substituted with one or more R²⁰.

In some embodiments, R¹ and R², taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰.

In some embodiments, R¹ and R², taken together with the atoms connecting them, form a monocyclic 5- to-6-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰.

In some embodiments, when one of X¹ and X² is NR¹ or NR², R¹ and R², taken together with the atoms connecting them, form a monocyclic or bicyclic 5- to-12-membered heterocycloalkyl ring containing no heteroatoms and/or heteroatomic groups cumulative with the N of X¹ or X², wherein the heterocycloalkyl ring is optionally substituted with one or more R²⁰.

In some embodiments, when one of X¹ and X² is NR¹ or NR², R¹ and R², taken together with the atoms connecting them, form a monocyclic 5- to-6-membered heterocycloalkyl ring containing no heteroatoms and/or heteroatomic groups cumulative with the N of X¹ or X², wherein the heterocycloalkyl ring is optionally substituted with one or more R²⁰.

In some embodiments, R¹ and R², taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X¹ and X², and wherein the heterocycloalkyl ring is optionally substituted with one or more R²⁰.

In certain embodiments of the foregoing, the monocyclic 5- to-6-membered heterocycloalkyl ring contains 1 O atom.

In certain embodiments when R¹ and R² are taken together to form a ring (e.g., monocyclic ring or bicyclic ring), R⁴ is H or CH₃.

In some embodiments, R² and R³ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X² and X³, and wherein the heteroaryl ring is optionally substituted with one or more R²⁰.

In some embodiments, R² and R³ are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

In some embodiments, R² and R³ are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S (e.g., O and NH), wherein the heteroatom or heteroatomic group is cumulative with the values selected for X² and X³, and wherein the heterocycloalkyl ring is optionally substituted with one or more R²⁰.

In some embodiments, R² and R³, taken together with the atoms connecting them, form a monocyclic or bicyclic C₄-C₁₂ cycloalkyl ring optionally substituted with one or more R²⁰.

In some embodiments, R² and R³, taken together with the atoms connecting them, form a monocyclic C₅-C₆ cycloalkyl ring optionally substituted with one or more R²⁰.

In some embodiments, R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

In some embodiments, R² and R³, taken together with the atoms connecting them, form a monocyclic or bicyclic 5- to-6-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

In some embodiments, when one of X² and X³ is NR² or NR³, R² and R³, taken together with the atoms connecting them, form a monocyclic or bicyclic 5- to-12-membered heterocycloalkyl ring containing no heteroatoms and/or heteroatomic groups cumulative with the N of X² or X³, wherein the heterocycloalkyl ring is optionally substituted with one or more R²⁰.

In some embodiments, when one of X² and X³ is NR² or NR³, R² and R³, taken together with the atoms connecting them, form a monocyclic 5- to-6-membered heterocycloalkyl ring containing no heteroatoms and/or heteroatomic groups cumulative with the N of X² or X³, wherein the heterocycloalkyl ring is optionally substituted with one or more R²⁰.

In some embodiments, R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X² and X³ wherein the heterocycloalkyl ring is optionally substituted with one or more R²⁰.

In certain embodiments of the foregoing, the monocyclic 5- to-6-membered heterocycloalkyl ring contains 1 O atom.

In certain embodiments when R² and R³ are taken together to form a ring (e.g., monocyclic ring or bicyclic ring), R⁴ is H or CH₃.

In some embodiments, R³ and R⁴ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heteroaryl ring is optionally substituted with one or more R²⁰.

In some embodiments, R³ and R⁴ are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

In some embodiments, R³ and R⁴ are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S (e.g., O and NH), wherein the heteroatom or heteroatomic group is cumulative with the values selected for X³ and X⁴, and wherein the heterocycloalkyl ring is optionally substituted with one or more R²⁰.

In some embodiments, R³ and R⁴, taken together with the atoms connecting them, form a monocyclic or bicyclic C₄-C₁₂ cycloalkyl ring optionally substituted with one or more R²⁰.

In some embodiments, R³ and R⁴, taken together with the atoms connecting them, form a monocyclic C₅-C₆ cycloalkyl ring optionally substituted with one or more R²⁰.

In some embodiments, R³ and R⁴, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

In some embodiments, R³ and R⁴, taken together with the atoms connecting them, form a monocyclic or bicyclic 5- to-6 membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

In some embodiments, when one of X³ and X⁴ is NR³ or NR⁴, R³ and R⁴, taken together with the atoms connecting them, form a monocyclic or bicyclic 5- to-12-membered heterocycloalkyl ring containing no heteroatoms and/or heteroatomic groups cumulative with the N of X³ or X⁴, wherein the heterocycloalkyl ring is optionally substituted with one or more R²⁰.

In some embodiments, when one of X³ and X⁴ is NR³ or NR⁴, R³ and R⁴, R³ and R⁴, taken together with the atoms connecting them, form a monocyclic 5- to-6-membered heterocycloalkyl ring containing no heteroatoms and/or heteroatomic groups cumulative with the N of X³ or X⁴, wherein the heterocycloalkyl ring is optionally substituted with one or more R²⁰.

In some embodiments, R³ and R⁴, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X³ and X⁴, and wherein the heterocycloalkyl ring is optionally substituted with one or more R²⁰.

In certain embodiments, monocyclic 5- to-6-membered heterocycloalkyl ring contains 1 O atom.

In certain embodiments, when R³ and R⁴ are taken together to form a ring, R² is H or CH₃ (e.g., H).

In some embodiments, R³ and R⁵ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In some embodiments, R³ and R⁵ are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R³ and R⁵ are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S (e.g., O and NH), wherein the heterocycloalkyl ring is optionally substituted with one or more R²⁰.

In some embodiments, R³ and R⁵, taken together with the atoms connecting them, form a monocyclic or bicyclic C₄-C₁₂ cycloalkyl ring optionally substituted with one or more R²⁰.

In some embodiments, R³ and R⁵, taken together with the atoms connecting them, form a monocyclic C₅-C₆ cycloalkyl ring optionally substituted with one or more R²⁰.

In some embodiments, R³ and R⁵, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R³ and R⁵, taken together with the atoms connecting them, form a monocyclic or bicyclic 5- to-6 membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R³ and R⁵, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heterocycloalkyl ring is optionally substituted with one or more R²⁰.

In certain embodiments, monocyclic 5- to-6-membered heterocycloalkyl ring contains 1 O atom.

In certain embodiments, when R³ and R⁵ are taken together to form a ring, each of R¹, R², and R⁶ is independently H, C₁-C₆ alkyl, C₁-C₆ alkoxy, or halo.

In some embodiments, R⁵ and R⁶ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In some embodiments, R⁵ and R⁶ are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R⁵ and R⁶ are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S (e.g., O and NH), wherein the heterocycloalkyl ring is optionally substituted with one or more R²⁰.

In some embodiments, R⁵ and R⁶, taken together with the atoms connecting them, form a monocyclic or bicyclic C₄-C₁₂ cycloalkyl ring optionally substituted with one or more R²⁰.

In some embodiments, R⁵ and R⁶, taken together with the atoms connecting them, form a monocyclic C₅-C₆ cycloalkyl ring optionally substituted with one or more R²⁰.

In some embodiments, R⁵ and R⁶, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R⁵ and R⁶, taken together with the atoms connecting them, form a monocyclic or bicyclic 5- to-6 membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R⁵ and R⁶, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heterocycloalkyl ring is optionally substituted with one or more R²⁰.

In certain embodiments, monocyclic 5- to-6-membered heterocycloalkyl ring contains 1 O atom.

In certain embodiments, when R⁵ and R⁶ are taken together to form a ring, each of R¹, R², and R³ is independently H, C₁-C₆ alkyl, C₁-C₆ alkoxy, or halo.

The Groups R¹, R², R³, R⁴, R⁵, and R⁶ when Formula AA is Formula AA-1

In some embodiments, R¹ and R², taken together with the atoms connecting them, form a monocyclic or bicyclic C₄-C₁₂ cycloalkyl ring optionally substituted with one or more R²⁰.

In some embodiments, R¹ and R², taken together with the atoms connecting them, form a monocyclic C₅-C₆ cycloalkyl ring optionally substituted with one or more R²⁰.

In some embodiments, R¹ and R², taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰.

In some embodiments, R¹ and R², taken together with the atoms connecting them, form a monocyclic or bicyclic 5- to-6-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰.

In some embodiments, R¹ and R², taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X¹ and X², and wherein the heterocycloalkyl ring is optionally substituted with one or more R²⁰.

In certain embodiments of the foregoing, the monocyclic 5- to-6-membered heterocycloalkyl ring contains 1 O atom.

In certain embodiments when R¹ and R² are taken together to form a ring (e.g., monocyclic ring or bicyclic ring), R⁴ is H or CH₃.

In some embodiments, R² and R³, taken together with the atoms connecting them, form a monocyclic or bicyclic C₄-C₁₂ cycloalkyl ring optionally substituted with one or more R²⁰.

In some embodiments, R² and R³, taken together with the atoms connecting them, form a monocyclic C₅-C₆ cycloalkyl ring optionally substituted with one or more R²⁰.

In some embodiments, R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

In certain embodiments, R² and R³, taken together with the atoms connecting them, form a monocyclic or bicyclic 5- to-12-membered heterocycloalkyl ring containing 1-3 heteroatoms and/or heteroatomic groups independently selected from O, N, NH, and NR¹³ and S, wherein the heteroatom or heteroatomic group is cumulative with the values selected for X² and X³, and wherein the heterocycloalkyl ring is optionally substituted with from 1-2 R²⁰.

In some embodiments, R² and R³, taken together with the atoms connecting them, form a monocyclic or bicyclic 5- to-6-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

In some embodiments, R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X² and X³, and wherein the heterocycloalkyl ring is optionally substituted with one or more R²⁰.

In certain embodiments of foregoing, the monocyclic 5- to-6-membered heterocycloalkyl ring contains 1 heteroatom or heteroatomic group selected from O and NH, wherein the heteroatom or heteroatomic group is cumulative with the values selected for X² and X³.

In certain embodiments of the foregoing, the monocyclic 5- to-6-membered heterocycloalkyl ring contains 1 O atom.

In certain embodiments when R² and R³ are taken together to form a ring (e.g., monocyclic ring or bicyclic ring), R⁴ is H or CH₃.

In some embodiments, R³ and R⁴, taken together with the atoms connecting them, form a monocyclic or bicyclic C₄-C₁₂ cycloalkyl ring optionally substituted with one or more R²⁰.

In some embodiments, R³ and R⁴, taken together with the atoms connecting them, form a monocyclic C₅-C₆ cycloalkyl ring optionally substituted with one or more R²⁰.

In some embodiments, R³ and R⁴, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

In some embodiments, R³ and R⁴, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X³ and X⁴, and wherein the heterocycloalkyl ring is optionally substituted with one or more R²⁰.

In certain embodiments of the foregoing, the monocyclic 5- to-6-membered heterocycloalkyl ring contains 1 O atom.

In certain embodiments when R³ and R⁴ are taken together to form a ring (e.g., monocyclic ring or bicyclic ring), R⁴ is H or CH₃.

In some embodiments, R³ and R⁵ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In some embodiments, R³ and R⁵ are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R³ and R⁵ are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S (e.g., O and NH), wherein the heterocycloalkyl ring is optionally substituted with one or more R²⁰.

In some embodiments, R³ and R⁵, taken together with the atoms connecting them, form a monocyclic or bicyclic C₄-C₁₂ cycloalkyl ring optionally substituted with one or more R²⁰.

In some embodiments, R³ and R⁵, taken together with the atoms connecting them, form a monocyclic C₅-C₆ cycloalkyl ring optionally substituted with one or more R²⁰.

In some embodiments, R³ and R⁵, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R³ and R⁵, taken together with the atoms connecting them, form a monocyclic or bicyclic 5- to-6 membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R³ and R⁵, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heterocycloalkyl ring is optionally substituted with one or more R²⁰.

In certain embodiments, monocyclic 5- to-6-membered heterocycloalkyl ring contains 1 O atom.

In certain embodiments, when R³ and R⁵ are taken together to form a ring, each of R¹, R², and R⁶ is independently H, C₁-C₆ alkyl, C₁-C₆ alkoxy, or halo.

In some embodiments, R⁵ and R⁶ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In some embodiments, R⁵ and R⁶ are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R⁵ and R⁶ are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S (e.g., O and NH), wherein the heterocycloalkyl ring is optionally substituted with one or more R²⁰.

In some embodiments, R⁵ and R⁶, taken together with the atoms connecting them, form a monocyclic or bicyclic C₄-C₁₂ cycloalkyl ring optionally substituted with one or more R²⁰.

In some embodiments, R⁵ and R⁶, taken together with the atoms connecting them, form a monocyclic C₅-C₆ cycloalkyl ring optionally substituted with one or more R²⁰.

In some embodiments, R⁵ and R⁶, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R⁵ and R⁶, taken together with the atoms connecting them, form a monocyclic or bicyclic 5- to-6 membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, R⁵ and R⁶, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heterocycloalkyl ring is optionally substituted with one or more R²⁰.

In certain embodiments, monocyclic 5- to-6-membered heterocycloalkyl ring contains 1 O atom.

In certain embodiments, when R⁵ and R⁶ are taken together to form a ring, each of R¹, R², and R³ is independently H, C₁-C₆ alkyl, C₁-C₆ alkoxy, or halo.

The Groups R¹, R², R³, and R⁴ when Formula AA is Formula AA-2

In some embodiments, R¹ and R² are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In certain embodiments, the ring is a monocyclic 5-6 membered cycloalkyl ring optionally substituted with one or more R²⁰, or a monocyclic 5-6-membered heterocycloalkyl ring containing 1-3 heteroatoms and/or heteroatomic groups independently selected from O, or NH, wherein the heterocycloalkyl ring is optionally substituted with one or more R²⁰.

In certain embodiments of the foregoing, the ring is a monocyclic 5-6-membered heterocycloalkyl ring containing one heteroatom or heteroatomic group independently selected from O, or NH, wherein the heterocycloalkyl ring is optionally substituted with one or more (e.g., two) R²⁰.

In some embodiments, R² and R³ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In certain embodiments, the ring is a monocyclic 5-6 membered cycloalkyl ring optionally substituted with one or more R²⁰, or a monocyclic 5-6-membered heterocycloalkyl ring containing 1-3 heteroatoms and/or heteroatomic groups independently selected from O, or NH, wherein the heterocycloalkyl ring is optionally substituted with one or more R²⁰.

In certain embodiments, the ring is a monocyclic 5-6-membered heterocycloalkyl ring containing one heteroatom or heteroatomic group independently selected from O or NH, wherein the heterocycloalkyl ring is optionally substituted with one or more (e.g., two) R²⁰.

Non-Limiting Combinations of X¹-X⁴ and R¹-R⁴
[1]

In some embodiments, X¹ is CR¹; X² is NR²; X³ is N; and/or X⁴ is CR⁴ (e.g., ring A is

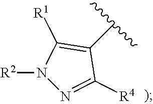

and

R¹ and R², taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the value selected for X², and wherein the heterocycloalkyl ring is optionally substituted with one or more R²⁰.

In certain embodiments of the foregoing, R¹ and R², taken together with the atoms connecting them, form a monocyclic 5- to-6-membered heterocycloalkyl ring containing 1 heteroatom or heteroatomic group selected from O, NH, and NR¹³ (e.g., O, NH, or NCH₃ (e.g., O)), wherein the heteroatom or heteroatomic group is cumulative with the value selected for X², and wherein the heterocycloalkyl ring is optionally substituted with one or more R²⁰. As a non-limiting example of the foregoing, the monocyclic 5- to-6-membered heterocycloalkyl ring contains 1 O atom (e.g., ring A is

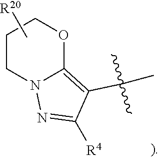

In certain other embodiments, R¹ and R², taken together with the atoms connecting them, form a monocyclic 5- to-6-membered heterocycloalkyl ring containing no heteroatoms or heteroatomic groups cumulative with the value selected for X², wherein the heterocycloalkyl ring is optionally substituted with one or more R²⁰. As a non-limiting example of the foregoing, the monocyclic 5- to-6-membered heterocycloalkyl ring contains 1 O atom (e.g., ring A is

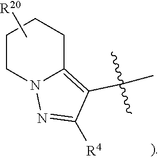

In certain embodiments of [1], R⁴ is H or CH₃.

In certain embodiments, A is

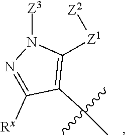

wherein R^{x} is selected from the group consisting of H and C₁-C₆ alkyl (e.g., methyl); Z¹ is selected from the group consisting of O, NH, and —CH₂-optionally substituted with 1-2 R²⁰; Z² is selected from the group consisting of NH and —CH₂-optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂-optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).

In certain embodiments of the foregoing, A is

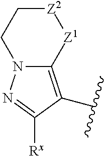

(e.g., Z¹ is O; and Z² is CH₂ optionally substituted with 1 R²⁰).
[2]

In some embodiments, X¹ is N; X² is NR²; X³ is CR³; and/or X⁴ is CR⁴ (e.g., ring A

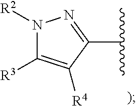

and

R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the value selected for X², and wherein the heterocycloalkyl ring is optionally substituted with one or more R²⁰.

In certain embodiments, R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the value selected for X², and wherein the heterocycloalkyl ring is optionally substituted with one or more R²⁰. As a non-limiting example of the foregoing, the monocyclic 5- to-6-membered heterocycloalkyl ring contains 1 O atom (e.g., ring A is

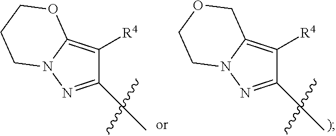

or the monocyclic 5- to-6-membered heterocycloalkyl ring contains 1 NH or NMe (e.g., the ring including X¹—X⁴ is

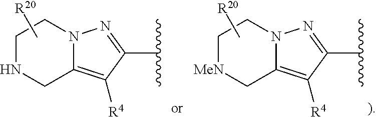

In certain embodiments of [2], R⁴ is H or CH₃.

In some embodiments, A is

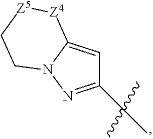

wherein Z⁴ is selected from the group consisting of —CH₂—, —C(O)—, and NH; Z⁵ is selected from the group consisting of O, NH, N—CH₃, and —CH₂-.

In some embodiments, A is selected from the group consisting of:

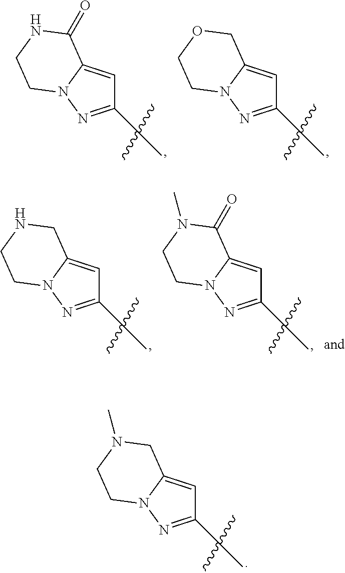 [SMILES: *c1cc2n(n1)CCNC2=O ; *c1cc2n(n1)CCOC2 ; *c1cc2n(n1)CCNC2 ; *c1cc2n(n1)CCN(C)C2=O ; *c1cc2n(n1)CCN(C)C2]

[3]

In some embodiments, X¹ is N; X² is NR²; X³ is CR³; and/or X⁴ is CR⁴ (e.g., ring A is

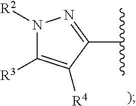

and

R³ and R⁴, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In certain embodiments, R³ and R⁴, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heterocycloalkyl ring is optionally substituted with one or more R²⁰. As a non-limiting example of the foregoing, the monocyclic 5- to-6-membered heterocycloalkyl ring contains 1 O atom (e.g., ring A is

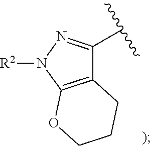

or the monocyclic 5- to-6-membered heterocycloalkyl ring contains 1 NH or NMe.

In some embodiments, X¹ is N; X² is NR²; X³ is CR³; and/or X⁴ is CR⁴ (e.g., ring A is

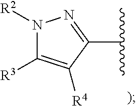

and

R³ and R⁴, taken together with the atoms connecting them, form a monocyclic or bicyclic C₄-C₁₂ cycloalkyl ring optionally substituted with one or more R²⁰.

In certain embodiments of [3], R² is H, C₁-C₆ alkyl, or C₁-C₆ haloalkyl (e.g., R² can be CF₂H).

[4]

In some embodiments, X¹ is S; X² is CR²; X³ is CR³; and/or X⁴ is CR⁴ (e.g., ring A is

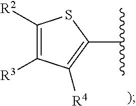

and

R³ and R⁴ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In certain embodiments of foregoing, R³ and R⁴ are taken together with the atoms connecting them to form a ring, wherein the ring is selected from the group consisting of:

(i) a monocyclic or bicyclic C₄-C₁₂ cycloalkyl ring optionally substituted with one or more R²⁰ and

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In certain embodiments, R³ and R⁴ are taken together with the atoms connecting them to form a ring, wherein the ring is selected from the group consisting of:

(i) a monocyclic C₅-C₆ cycloalkyl ring optionally substituted with one or more R²⁰ and

(ii) a monocyclic 5- to 6-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In some embodiments, X¹ is S; X² is CR²; X³ is CR³; and/or X⁴ is CR⁴ (e.g., ring A is

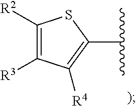

and

R³ and R⁴, taken together with the atoms connecting them, form a monocyclic or bicyclic C₄-C₁₂ cycloalkyl ring optionally substituted with one or more R²⁰.

In certain embodiments of the foregoing, R³ and R⁴, taken together with the atoms connecting them, form a monocyclic C₅-C₆ cycloalkyl ring optionally substituted with one or more R²⁰.

In some embodiments, X¹ is S; X² is CR²; X³ is CR³; and/or X⁴ is CR⁴ (e.g., ring A is

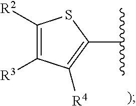

and

R³ and R⁴, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In certain embodiments of the foregoing, R³ and R⁴, taken together with the atoms connecting them, form a monocyclic 5- to-6-membered heterocycloalkyl ring containing 1 heteroatom or heteroatomic group selected from O, N, NH, and NR¹³ (e.g., O, N, NH, or NCH₃), wherein the heterocycloalkyl ring is optionally substituted with one or more R²⁰. As a non-limiting example of the foregoing, the monocyclic 5- to-6-membered heterocycloalkyl ring contains 1 O atom.

In certain embodiments of [4], R² is H or CH₃.

In certain embodiments of [4], R²⁰ is C₁-C₆ alkyl (e.g., methyl).

[5]

In some embodiments, X¹ is S; X² is CR²; X³ is CR³; and/or X⁴ is CR⁴ (e.g., ring A is

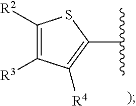

and

R² and R³ are taken together with the atoms connecting them to form a ring, wherein the ring is selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

In certain embodiments of the foregoing, the ring is selected from:

(i) a monocyclic C₅-C₆ cycloalkyl ring optionally substituted with one or more R²⁰ and

(ii) a monocyclic 5- to-6-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In certain embodiments, the ring is a monocyclic 5-6 membered cycloalkyl ring optionally substituted with one or more R²⁰.

In certain embodiments of [5], each R²⁰ is independently selected from hydroxy, C₁-C₆ alkyl (e.g., methyl), and NR⁸R⁹.

[6]

In some embodiments, X¹ is S; X² is CR²; X³ is CR³; and/or X⁴ is N (e.g., ring A is

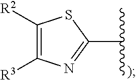

and

R² and R³, taken together with the atoms connecting them, form a monocyclic or bicyclic C₄-C₁₂ cycloalkyl ring optionally substituted with one or more R²⁰.

In certain embodiments of the foregoing, R² and R³, taken together with the atoms connecting them, form a monocyclic or bicyclic C₅-C₆ cycloalkyl ring optionally substituted with one or more R²⁰.

In some embodiments, X¹ is S; X² is CR²; X³ is CR³; and/or X⁴ is N (e.g., ring A is

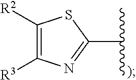

and

R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In certain embodiments of the foregoing, R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heterocycloalkyl ring is optionally substituted with one or more R²⁰. As a non-limiting example of the foregoing, the monocyclic 5- to-6-membered heterocycloalkyl ring can contain 1 heteroatom or heteroatomic group selected from O and NH.

[7]

In some embodiments, X¹ is N; X² is CR²; X³ is NR³; and/or X⁴ is CR⁴ (e.g., ring A is

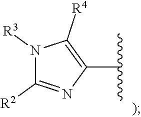

and

R³ and R⁴, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰.

In certain embodiments of the foregoing, R³ and R⁴, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heterocycloalkyl ring is optionally substituted with one or more R²⁰. As a non-limiting example of the foregoing, the monocyclic 5- to-6-membered heterocycloalkyl ring contains 1 O atom (e.g., ring A is

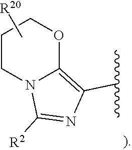

In certain embodiments of [1], R² is H or CH₃.

### The Variable R²⁰

In some embodiments, R²⁰ is selected from the group consisting of: hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom, wherein the ring is optionally substituted with OS(O)₂Ph.

In some embodiments, R²⁰ is hydroxy,

In some embodiments, R²⁰ is halo (e.g., fluoro).

In some embodiments, R²⁰ is oxo,

In some embodiments, R²⁰ is C₁-C₆ alkyl (e.g., methyl or ethyl) optionally substituted with one R²¹.

In some embodiments, R²⁰ is C₁-C₆ alkoxy (e.g., methoxy, ethoxy, or isopropoxy) optionally substituted with one R²¹.

In some embodiments, R²⁰ is NR⁸R⁹ (e.g., NHMe, NHEt, NH₂, NHBoc, NMeBoc).

In some embodiments, R²⁰ is 3- to 10-membered heterocycloalkyl (e.g., azetidinyl or pyrrolidinyl) optionally substituted with one R²¹.

In some embodiments, R²⁰ is or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom, wherein the ring is optionally substituted with OS(O)₂Ph (e.g., the ring can be

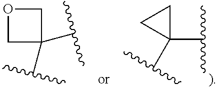 [SMILES: *C1(*)COC1 ; *C1(*)CC1]

The Variable R²⁰ when Formula AA is Formula AA-1

In some embodiments, R²⁰ is selected from the group consisting of: hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom optionally substituted with OS(O)₂Ph.

In some embodiments, R²⁰ is hydroxy,

In some embodiments, R²⁰ is halo (e.g., fluoro).

In some embodiments, R²⁰ is oxo,

In some embodiments, R²⁰ is C₁-C₆ alkyl (e.g., methyl or ethyl) optionally substituted with one R²¹.

In some embodiments, R²⁰ is C₁-C₆ alkoxy (e.g., methoxy, ethoxy, or isopropoxy) optionally substituted with one R²¹.

In some embodiments, R²⁰ is NR⁸R⁹ (e.g., NHMe, NHEt, NH₂, NHBoc, NMeBoc).

In some embodiments, R²⁰ is 3- to 10-membered heterocycloalkyl (e.g., azetidinyl or pyrrolidinyl) optionally substituted with one R²¹.

In some embodiments, R²⁰ is or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom, wherein the ring is optionally substituted with OS(O)₂Ph (e.g., the ring can be or

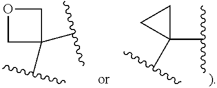 [SMILES: *C1(*)COC1 ; *C1(*)CC1]

The Variable R²⁰ when Formula AA is Formula AA-2

In some embodiments (e.g., when R¹ and R² are taken together to form a ring), R²⁰ is C₁-C₆ alkyl (e.g., methyl).

In some embodiments (e.g., when R² and R³ are taken together to form a ring), R²⁰ is selected from hydroxy, C₁-C₆ alkyl (e.g., methyl), and NR⁸R⁹.

In some embodiments, R²⁰ is hydroxyl.

In some embodiments, R²⁰ is C₁-C₆ alkyl (e.g., methyl).

In some embodiments, R²⁰ is NR⁸R⁹.

In some embodiments, R²⁰ is halo.

The variable R²¹

In some embodiments of Formula AA, AA-1 or AA-2, R²¹ is selected from the group consisting of halo (e.g., fluoro), NR⁸R⁹, C₂-C₆ alkynyl (e.g., ethynyl), and C₁-C₆ alkoxy (e.g., methoxy).

In some embodiments of Formula AA, AA-1 or AA-2, R²¹ is halo (e.g., fluoro).

In some embodiments of Formula AA, AA-1 or AA-2, R²¹ is NR⁸R⁹. In certain embodiments of the foregoing, each of R⁸ and R⁹ is independently hydrogen or C₁-C₆ alkyl (e.g., R²¹ is NHMe, NH₂, or NMe₂). In certain embodiments, one of R⁸ and R⁹ is selected from COR¹³ and CO₂R¹³ (e.g, R²¹ is NHBoc; or R²¹ is NHAc or NHC(═O)CF₃).

In some embodiments of Formula AA, AA-1 or AA-2, R²¹ is C₂-C₆ alkynyl (e.g., ethynyl).

In some embodiments of Formula AA, AA-1 or AA-2, R²¹ is C₁-C₆ alkoxy (e.g., methoxy).

In certain embodiments, R²¹ is selected from the group consisting of F, NH₂, NHMe, NMe₂, NHBoc, NMeBoc, NHAc, ethynyl, and OMe.

### The Variable R²²

In some embodiments, R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³.

In some embodiments, R²² at each occurrence is independently selected from the group consisting of: hydroxy, halo, CN, oxo, C₁-C₆ alkyl optionally substituted with one R²³, C₁-C₆ alkoxy optionally substituted with one R²³, NR⁸R⁹, COOC₁-C₆ alkyl optionally substituted with one R²³, CONR⁸R⁹, 3- to 7-membered heterocycloalkyl optionally substituted with one R²³, C₆-C₁₀ aryl optionally substituted with one R²⁴, 5- to 10-membered heteroaryl optionally substituted with one R²⁴, NHCOC₁-C₆ alkyl optionally substituted with one R²³, NHCOC₆-C₁₀ aryl optionally substituted with R²⁴, NHCO(5- to 10-membered heteroaryl) optionally substituted with one R²⁴, NHCO(3- to 7-membered heterocycloalkyl) optionally substituted with one R²³, and NHCOC₂-C₆ alkynyl optionally substituted with one or more R²³.

In some embodiments, R²² at each occurrence is independently selected from the group consisting of: hydroxy, halo, CN, oxo, C₁-C₆ alkyl, C₁-C₆ alkoxy, NR⁸R⁹, COOC₁-C₆ alkyl, CONR⁸R⁹, 3- to 7-membered heterocycloalkyl, C₆-C₁₀ aryl, 5- to 10-membered heteroaryl, NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl.

In some embodiments, R²² at each occurrence is independently selected from the group consisting of: hydroxy, halo, CN, oxo, C₁-C₆ alkyl, C₁-C₆ alkoxy, NR⁸R⁹, COOC₁-C₆ alkyl, CONR⁸R⁹, 3- to 7-membered heterocycloalkyl, C₆-C₁₀ aryl, 5- to 10-membered heteroaryl, and NHCOC₁-C₆ alkyl.

In some embodiments, R²² at each occurrence is selected from the group consisting of hydroxyl, halo (e.g., fluoro), CN, NR⁸R⁹, C₂-C₆ alkynyl (e.g., ethynyl), and C₁-C₆ alkoxy (e.g., methoxy).

### The Variable R²³

In some embodiments, R²³ at each occurrence is independently selected from the group consisting of: hydroxy, halo, NR⁸R⁹, C₁-C₄ alkyl, OC₁-C₄ alkyl, and oxo.

In some embodiments, R²³ at each occurrence is independently selected from the group consisting of: hydroxy, halo, NR⁸R⁹, C₁-C₄ alkyl, and OC₁-C₄ alkyl.

### The Variable R²⁴

In some embodiments, R²⁴ at each occurrence is independently selected from the group consisting of: hydroxy, halo, NR⁸R⁹, C₁-C₄ alkyl, OC₁-C₄ alkyl, and oxo.

In some embodiments, R²⁴ at each occurrence is independently selected from the group consisting of: hydroxy, halo, NR⁸R⁹ (e.g., NHC(O)R¹³, NH₂, NH(C₁-C₄ alkyl), N(C₁-C₄ alkyl)₂, NHC(O)OR¹³), C₁-C₄ alkyl, and OC₁-C₄ alkyl.

### The Variable R²⁵

In some embodiments, R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶.

In some embodiments, R²⁵ at each occurrence is independently selected from the group consisting of: hydroxy, halo, CN, oxo, C₁-C₆ alkyl optionally substituted with one or more R²⁶, C₁-C₆ alkoxy, NR⁸R⁹, COOC₁-C₆ alkyl, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), C₆-C₁₀ aryloxy, and S(O₂)C₁-C₆ alkyl.

In some embodiments, R²⁵ at each occurrence is independently selected from the group consisting of: hydroxy, halo, CN, oxo, C₁-C₆ alkyl, C₁-C₆ alkoxy, NR⁸R⁹ (e.g., NHC(O)R¹³, NH₂, NH(C₁-C₄ alkyl), N(C₁-C₄ alkyl)₂, NHC(O)OR¹³), COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), C₆-C₁₀ aryloxy, and S(O₂)C₁-C₆ alkyl.

### The Variable R²⁶

In some embodiments, R²⁶ at each occurrence is independently selected from the group consisting of: hydroxy, halo, C₆-C₁₀ aryl, NR⁸R⁹, C₁-C₆ alkyl, and OC₁-C₆ alkyl.

In some embodiments, R²⁶ at each occurrence is independently selected from the group consisting of: hydroxy, halo, phenyl, NR⁸R⁹, C₁-C₄ alkyl, and OC₁-C₄ alkyl.

In some embodiments, R²⁶ at each occurrence is independently selected from the group consisting of: hydroxy, halo, NR⁸R⁹, C₁-C₄ alkyl, and OC₁-C₄ alkyl.

In some embodiments, R²⁶ at each occurrence is independently selected from the group consisting of: hydroxy, halo, NHC(O)R¹³, NH₂, NH(C₁-C₄ alkyl), N(C₁-C₄ alkyl)₂, NHC(O)OR¹³, C₁-C₄ alkyl, and OC₁-C₄ alkyl.

### The Variable R²⁷

In some embodiments, R²⁷, at each occurrence, is independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In certain embodiments, R²⁷, at each occurrence, is independently hydroxyl, oxo, halo (e.g., F), or C₁-C₆ alkyl. As a non-limiting example of the foregoing, R²⁷ at each occurrence can be independently C₁-C₆ alkyl.

The Variables o, o′, o″, p, p′, p″, and p′″
In some embodiments, o=1 or 2.
In some embodiments, o=1.
In some embodiments, o=2.
In some embodiments, p=0, 1, 2, or 3.
In some embodiments, p=0.
In some embodiments, p=1.
In some embodiments, p=2.
In some embodiments, o=1 and p=0.
In some embodiments, o=2 and p=0.
In some embodiments, o=1 and p=1.
In some embodiments, o=1 and p=2.
In some embodiments, o=2 and p=1.
In some embodiments, o=2 and p=2.
In some embodiments, o=2 and p=3.
In some embodiments, o′=0 or 1.
In some embodiments, o′=0.
In some embodiments, o′=1.
In some embodiments, p′=0 or 1.
In some embodiments, p′=0.
In some embodiments, p′=1.
In some embodiments, o′=1 and p′=0.
In some embodiments, o′=1 and p′=1.
In some embodiments, o′=0 and p′=1.
In some embodiments, o′=0 and p′=0.
In some embodiments, o″=0 or 1.
In some embodiments, o″=0.
In some embodiments, o″=1.
In some embodiments, p″=0, 1, or 2.
In some embodiments, p″=0.
In some embodiments, p″=1.
In some embodiments, o″=1 and p″=0.
In some embodiments, o″=1 and p″=1.
In some embodiments, o″=1 and p″=2.
In some embodiments, o″=0 and p″=1.
In some embodiments, o″=0 and p″=0.
In some embodiments, o″=0 and p″=2.
In some embodiments p′″=1, 2, or 3.
In some embodiments p′″=1.
In some embodiments p′″=2.
In some embodiments p′″=3.
In some embodiments, p′″=1 or 2.
The Variables t and t′
In some embodiments, t is 0, 1, 2, 3, 4, 5, or 6.
In some embodiments, t is 0, 1, 2, or 3.
In some embodiments, t is 0, 1, or 2.
In some embodiments, t is 0.
In some embodiments, t is 1.
In some embodiments, t is 2.
In some embodiments, t′ is 0, 1, 2, 3, or 4.
In some embodiments, t′ is 0, 1, 2, or 3.
In some embodiments, t′ is 0, 1, or 2.
In some embodiments, t′ is 0.
In some embodiments, t′ is 1.
In some embodiments, t′ is 2.

### The Ring B and Substitutions on the Ring B

In some embodiments, B is one of the rings disclosed hereinbelow, substituted as disclosed hereinbelow, wherein in each case the bond that is shown as being broken by the wavy line [symbol] connects B to the NHC(O) group of Formula AA.
In some embodiments, the optionally substituted ring B is

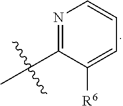

In some embodiments, the optionally substituted ring B is

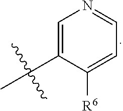

In some embodiments, the optionally substituted ring B is

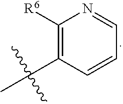

In some embodiments, the optionally substituted ring B is

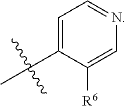

In some embodiments, the optionally substituted ring B is

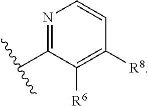

In some embodiments, the optionally substituted ring B is

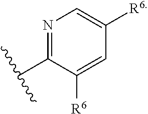

In some embodiments, the optionally substituted ring B is

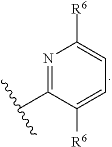

In some embodiments, the optionally substituted ring B is

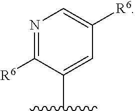

In some embodiments, the optionally substituted ring B is

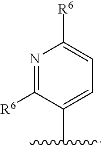

In some embodiments, the optionally substituted ring B is

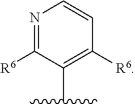

In some embodiments, the optionally substituted ring B is

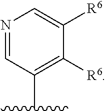

In some embodiments, the optionally substituted ring B is

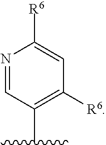

In some embodiments, the optionally substituted ring B is

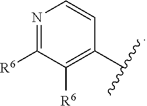

In some embodiments, the optionally substituted ring B is

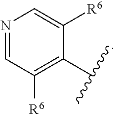

In some embodiments, the optionally substituted ring B is

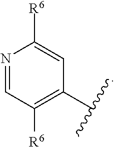

In some embodiments, the optionally substituted ring B is

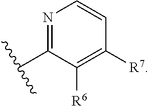

In some embodiments, the optionally substituted ring B is

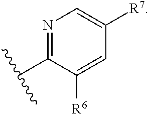

In some embodiments, the optionally substituted ring B is

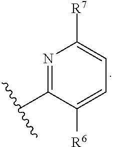

In some embodiments, the optionally substituted ring B is

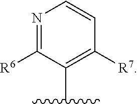

In some embodiments, the optionally substituted ring B is

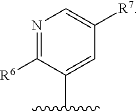

In some embodiments, the optionally substituted ring B is

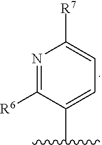

In some embodiments, the optionally substituted ring B is

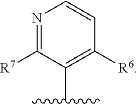

In some embodiments, the optionally substituted ring B is

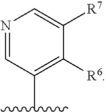

In some embodiments, the optionally substituted ring B is

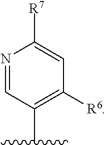

In some embodiments, the optionally substituted ring B is

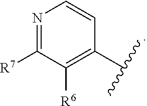

In some embodiments, the optionally substituted ring B is

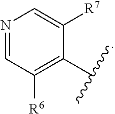

In some embodiments, the optionally substituted ring B is

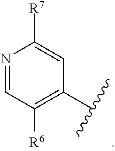

In some embodiments, the optionally substituted ring B is

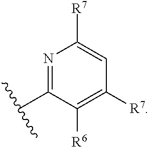

In some embodiments, the optionally substituted ring B is

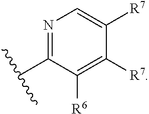

In some embodiments, the optionally substituted ring B is

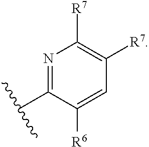

In some embodiments, the optionally substituted ring B is

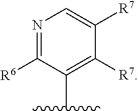

In some embodiments, the optionally substituted ring B is

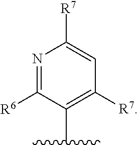

In some embodiments, the optionally substituted ring B is

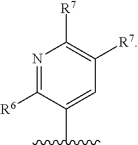

In some embodiments, the optionally substituted ring B is

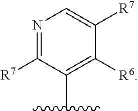

In some embodiments, the optionally substituted ring B is

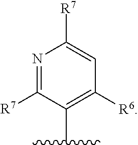

In some embodiments, the optionally substituted ring B is

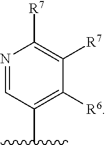

In some embodiments, the optionally substituted ring B is

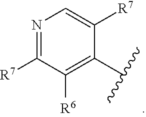

In some embodiments, the optionally substituted ring B is

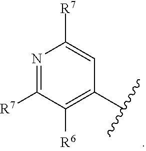

In some embodiments, the optionally substituted ring B is

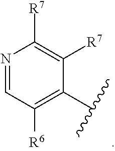

In some embodiments, the optionally substituted ring B is

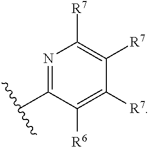

In some embodiments, the optionally substituted ring B is

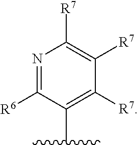

In some embodiments, the optionally substituted ring B is

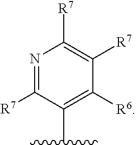

In some embodiments, the optionally substituted ring B is

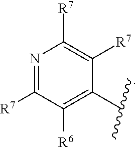

In some embodiments, the optionally substituted ring B is

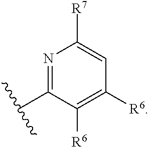

In some embodiments, the optionally substituted ring B is

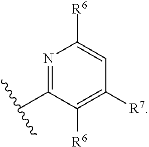

In some embodiments, the optionally substituted ring B is

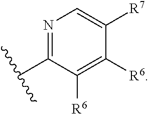

In some embodiments, the optionally substituted ring B is

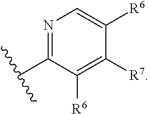

In some embodiments, the optionally substituted ring B is

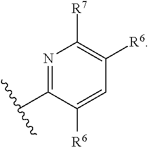

In some embodiments, the optionally substituted ring B is

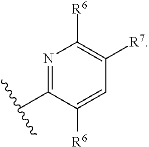

In some embodiments, the optionally substituted ring B is

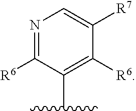

In some embodiments, the optionally substituted ring B is

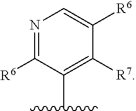

In some embodiments, the optionally substituted ring B is

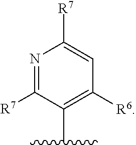

In some embodiments, the optionally substituted ring B is

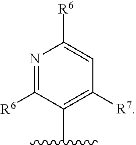

In some embodiments, the optionally substituted ring B is

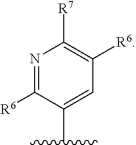

In some embodiments, the optionally substituted ring B is

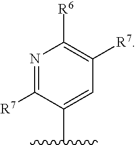

In some embodiments, the optionally substituted ring B is

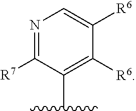

In some embodiments, the optionally substituted ring B is

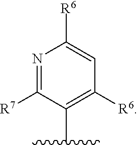

In some embodiments, the optionally substituted ring B is

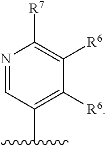

In some embodiments, the optionally substituted ring B is

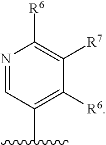

In some embodiments, the optionally substituted ring B is

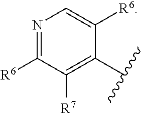

In some embodiments, the optionally substituted ring B is

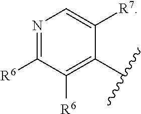

In some embodiments, the optionally substituted ring B is

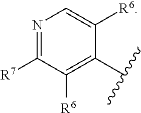

In some embodiments, the optionally substituted ring B is

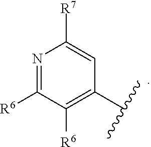

In some embodiments, the optionally substituted ring B is

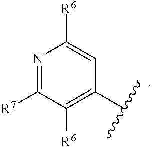

In some embodiments, the optionally substituted ring B is

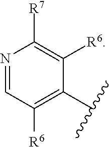

In some embodiments, the optionally substituted ring B is

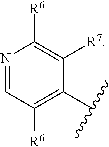

In some embodiments, the optionally substituted ring B is

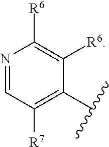

In some embodiments, the optionally substituted ring B is

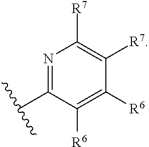

In some embodiments, the optionally substituted ring B is

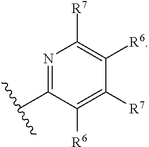

In some embodiments, the optionally substituted ring B is

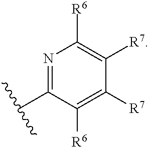

In some embodiments, the optionally substituted ring B is

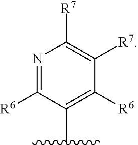

In some embodiments, the optionally substituted ring B is

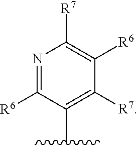

In some embodiments, the optionally substituted ring B is

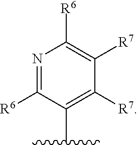

In some embodiments, the optionally substituted ring B is

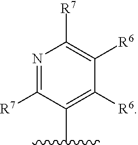

In some embodiments, the optionally substituted ring B is

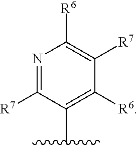

In some embodiments, the optionally substituted ring B is

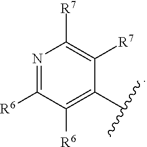

In some embodiments, the optionally substituted ring B is

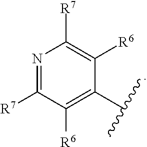

In some embodiments, the optionally substituted ring B is

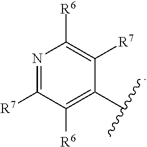

In some embodiments, the optionally substituted ring B is

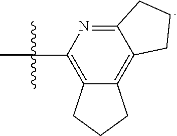 [SMILES: *c1nc2c(c3c1CCC3)CCC2]

In some embodiments, the optionally substituted ring B is selected from the group consisting of:

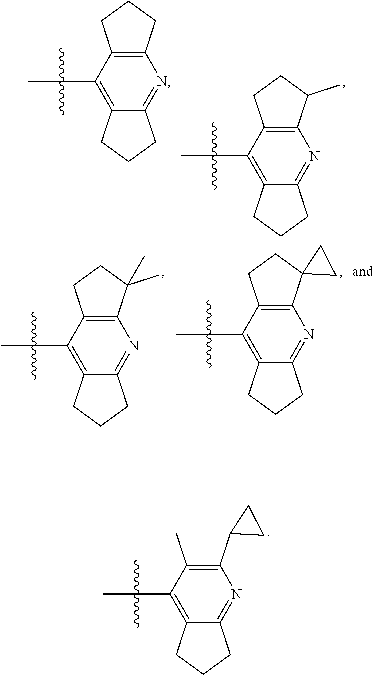

In some embodiments, the optionally substituted ring B is

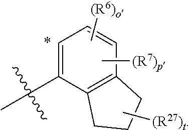

In certain embodiments of the foregoing, the optionally substituted ring B is

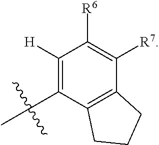

As a non-limiting example of the foregoing,

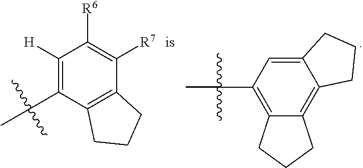

In some embodiments, the optionally substituted ring B is

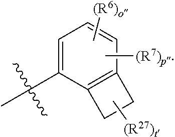

In certain embodiments of the foregoing, the optionally substituted ring B is

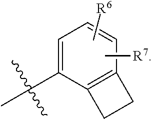

In certain embodiments of the foregoing,

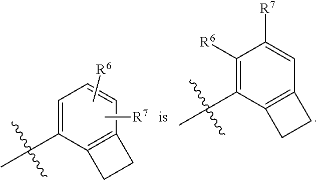

As non-limiting examples of the foregoing,

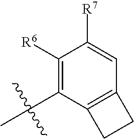

is selected from

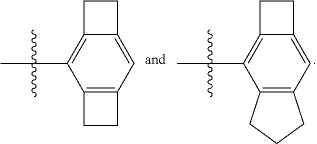 [SMILES: *c1c2c(cc3c1CC3)CC2 ; *c1c2c(cc3c1CC3)CCC2]

In some embodiments, the optionally substituted ring B is

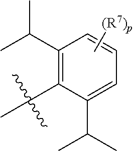

(e.g., the optionally substituted ring B can be

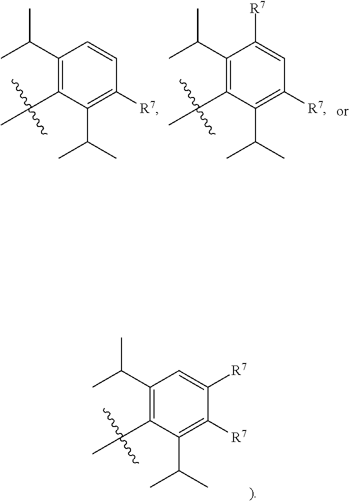

In some embodiments (when X¹ is S), the optionally substituted ring B is

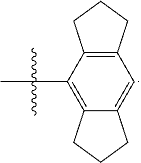 [SMILES: *c1c2c(cc3c1CCC3)CCC2]

In some embodiments (when X¹ is S), the optionally substituted ring B is

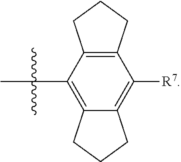

In some embodiments, the substituted ring B is

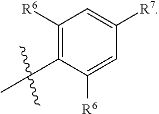

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl, C₆-C₁₀ aryl, and 5- to 10-membered heteroaryl, and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy.

In certain of the foregoing embodiments, one R⁶ is C₁-C₆ alkyl (e.g., isopropyl).

In certain of these embodiments, the other R⁶ is C₁-C₆ alkyl. For example, each R⁶ is isopropyl (i.e., the substituted ring B is

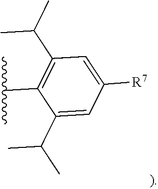

In certain other embodiments, one R⁶ is C₁-C₆ alkyl; and the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₁-C₆ alkyl; and the other R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 (e.g., 6) membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

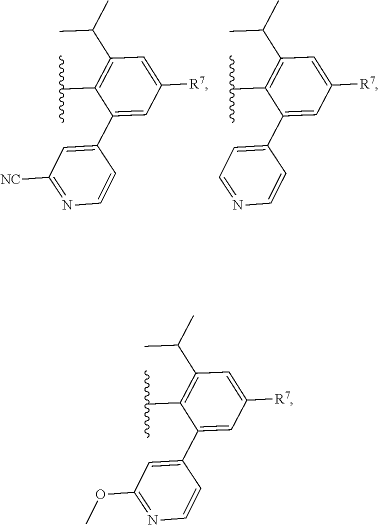

(e.g., R⁷ is halo (e.g., fluoro)).

In some embodiments, B is

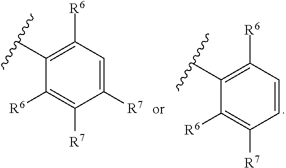

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl, C₆-C₁₀ aryl, and 5- to 10-membered heteroaryl, and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy.

In certain of these embodiments, one R⁶ is C₁-C₆ alkyl; and the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₁-C₆ alkyl; and the other R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 (e.g., 6) membered heteroaryl (e.g., pyridinyl (e.g., pyridin-4-yl), pyrimidinyl, pyridazinyl, oxazolyl, or thiazolyl) optionally substituted with a substituent selected from hydroxyl, halo, CN, C₁-C₆ alkyl, and C₁-C₆ alkoxy.

In certain of the foregoing embodiments, each R⁷ is independently C₁-C₆ alkyl, C₁-C₆ haloalkyl, halo, or cyano,

As a non-limiting example of the foregoing embodiments, substituted ring B is:

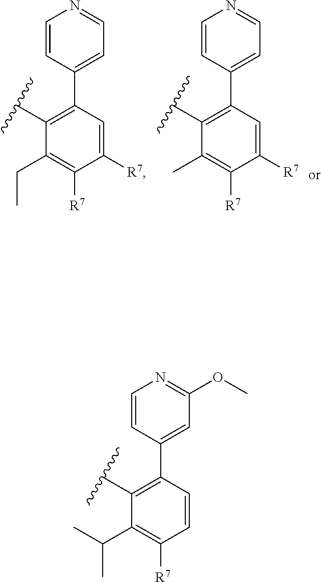

(e.g., each R⁷ is independently C₁-C₆ alkyl, C₁-C₆ haloalkyl, halo, or cyano).
The Optionally Substituted Ring B when Formula AA is Formula AA-1
In some embodiments,

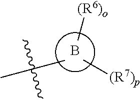

is selected from the group consisting of:

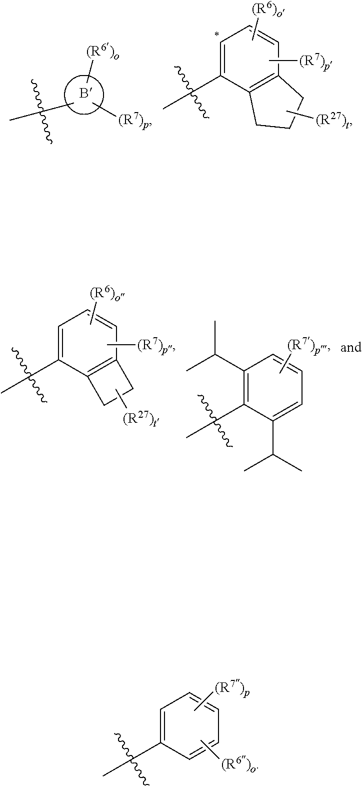

In some embodiments,

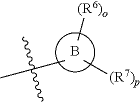

is selected from the group consisting of:

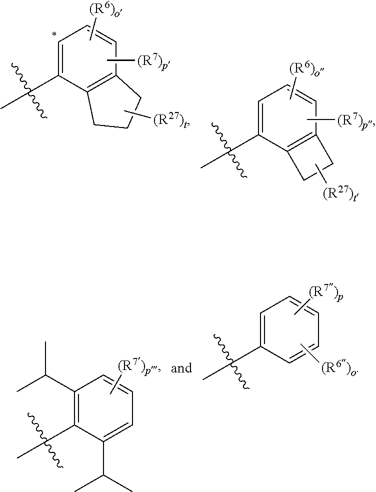

In some embodiments,

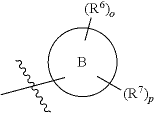

is selected from the group consisting of:

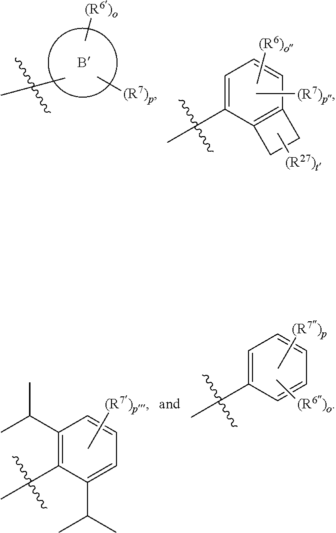

In some embodiments,

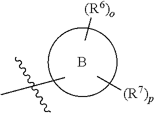

is selected from the group consisting of:

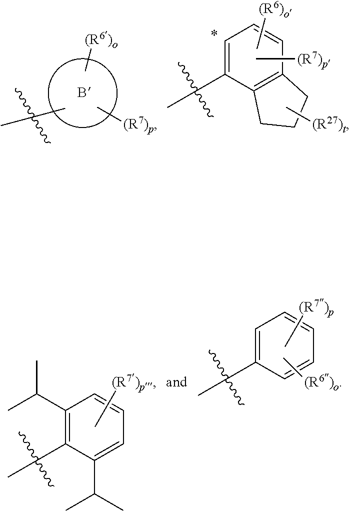

In some embodiments,

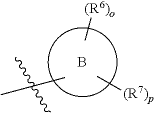

is selected from the group consisting of:

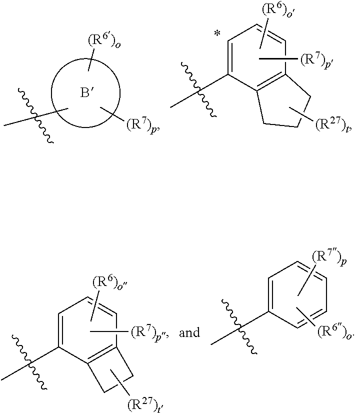

In some embodiments,

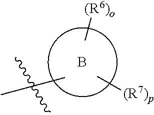

is selected from the group consisting of:

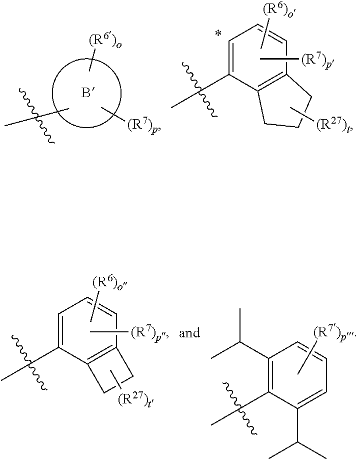

In some embodiments,

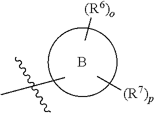

is selected from the group consisting of:

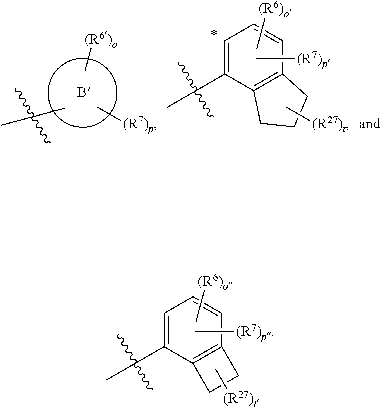

In some embodiments,

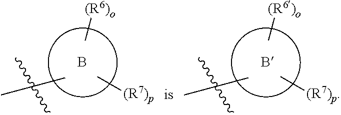

In some embodiments, B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members; 4-pyrimidinyl; 5-pyrimidinyl; 6-pyrimidinyl; pyridazinyl; pyrazinyl; 1,2,3-triazinyl; 1,2,4-triazinyl; tetrazinyl; imidazolyl; pyrazolyl; 1,2,3-triazolyl; tetrazolyl; or C₇-C₁₀ aryl In some embodiments, B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members; 5-pyrimidinyl; 6-pyrimidinyl; pyridazinyl; pyrazinyl; 1,2,3-triazinyl; 1,2,4-triazinyl; tetrazinyl; imidazolyl; pyrazolyl; 1,2,3-triazolyl; tetrazolyl; or C₇-C₁₀ aryl.
In some embodiments, B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members; 4-pyrimidinyl; 5-pyrimidinyl; 6-pyrimidinyl; pyridazinyl; pyrazinyl; 1,2,3-triazinyl; 1,2,4-triazinyl; tetrazinyl; imidazolyl; pyrazolyl; 1,2,3-triazolyl; tetrazolyl; or C₇-C₁₀ aryl; and wherein at least one R⁶ is ortho to the bond connecting the B′ ring to the NH(CO) group of Formula AA.
In some embodiments, B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members.
In certain embodiments of foregoing, B′ is pyridyl (e.g., 2-pyridyl or 4-pyridyl).
In some embodiments, B′ is 2-pyridyl.
In certain embodiments, the optionally substituted ring B′ is

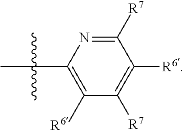

As a non-limiting example of the foregoing, the optionally substituted ring B′ is

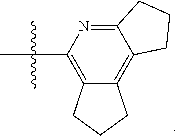 [SMILES: *c1nc2c(c3c1CCC3)CCC2]

In some embodiments, B′ is 4-pyridyl.

In certain embodiments, the optionally substituted ring B′ is

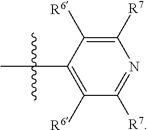

In certain embodiments,

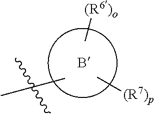

is selected from the group consisting of:

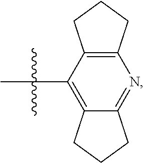 [SMILES: *c1c2c(nc3c1CCC3)CCC2]

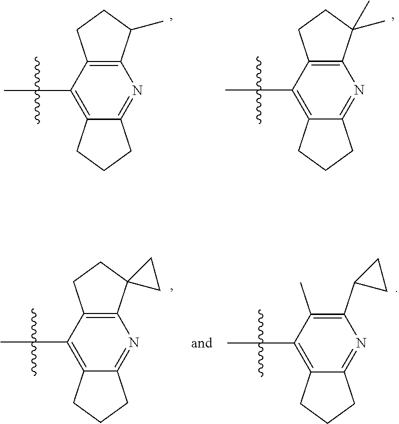 [SMILES: *c1c2c(nc3c1CCC3C)CCC2 ; *c1c2c(nc3c1CCC3(C)C)CCC2 ; *c1c2c(nc3c1CCC31CC1)CCC2 ; *c1c(C)c(C2CC2)nc2c1CCC2]

In some embodiment,

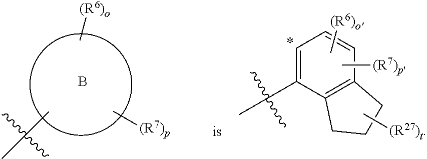

In certain embodiments,

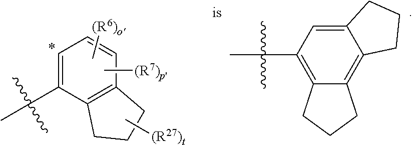

In some embodiments,

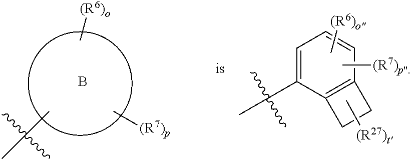

In certain embodiments,

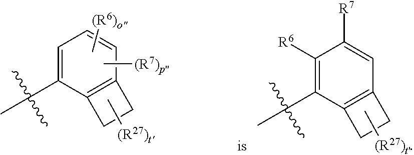

As non-limiting examples of the foregoing,

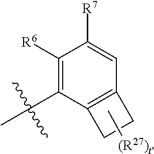

is selected from

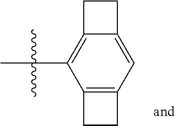 [SMILES: *c1c2c(cc3c1CC3)CC2]

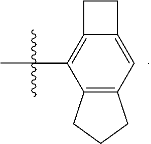 [SMILES: *c1c2c(cc3c1CC3)CCC2]

In some embodiments,

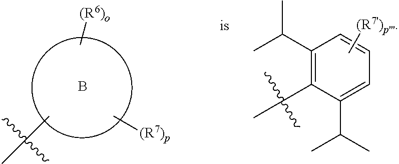

In some embodiments,

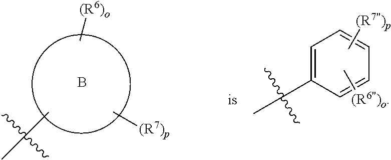

The Optionally Substituted Ring B when Formula AA is Formula AA-2
In some embodiments, the optionally substituted ring B is

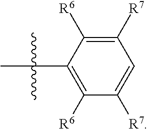

In some embodiments, the optionally substituted ring B is

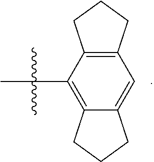 [SMILES: *c1c2c(cc3c1CCC3)CCC2]

In some embodiments, the optionally substituted ring B is other than

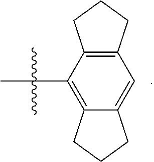 [SMILES: *c1c2c(cc3c1CCC3)CCC2]

In some embodiments, the optionally substituted ring B is

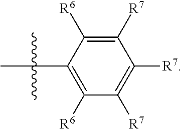

In some embodiments, the optionally substituted ring B is

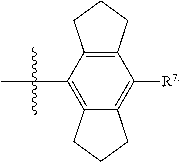

The Groups R⁶, R^{6′}, R⁷, and R^{7′}
The Groups R⁶ and R⁷

In some embodiments, at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

In some embodiments, R⁶ and R⁷, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

In some embodiments, R⁶ and R⁷, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

In some embodiments, two pairs of R⁶ and R⁷ on adjacent atoms, taken together with the atoms connecting them, independently form two C₄-C₈ carbocyclic rings or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

In some embodiments, two pairs of R⁶ and R⁷ on adjacent atoms, taken together with the atoms connecting them, independently form two C₅ carbocyclic rings optionally independently substituted with one or more R²⁷.

In some embodiments, two pairs of R⁶ and R⁷ on adjacent atoms, taken together with the atoms connecting them, independently form two C₄-C₈ carbocyclic ring or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

In some embodiments, two pairs of R⁶ and R⁷ on adjacent atoms, taken together with the atoms connecting them, independently form two C₅ carbocyclic rings optionally independently substituted with one or more R²⁷.

The Groups R⁶ and R⁷ when Formula AA is Formula AA-1 or Formula AA-2

In some embodiments, R⁶ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵.

In some embodiments, R⁷ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵.

In some embodiments, at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

In some embodiments, R⁶ and R⁷, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

In some embodiments, R⁶ and R⁷, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

In some embodiments, two pairs of R⁶ and R⁷ on adjacent atoms, taken together with the atoms connecting them, independently form two C₄-C₈ carbocyclic rings or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

In some embodiments, two pairs of R⁶ and R⁷ on adjacent atoms, taken together with the atoms connecting them, independently form two C₅ carbocyclic rings optionally independently substituted with one or more R²⁷.

In some embodiments, two pairs of R⁶ and R⁷ on adjacent atoms, taken together with the atoms connecting them, independently form two C₄-C₈ carbocyclic ring or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

In some embodiments, two pairs of R⁶ and R⁷ on adjacent atoms, taken together with the atoms connecting them, independently form two C₅ carbocyclic rings optionally independently substituted with one or more R²⁷.

The Groups R and R when Formula AA is Formula AA-1 and

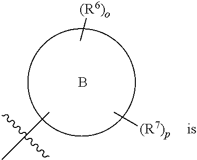

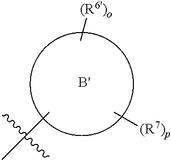

In some embodiments, at least one pair of R^{6′} and R⁷ on adjacent atoms, taken together with the atoms connecting them, independently form at least one C₄-C₈ (e.g, C₄-C₅ or C₇-C₈) cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷.

In some embodiments, two pairs of R^{6′} and R⁷ on adjacent atoms, taken together with the atoms connecting them, independently form a C₄-C₈ cycloalkyl ring or a 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷.

In some embodiments, at least one R⁶ is ortho to the bond connecting the B′ ring to the NH(CO) group of Formula AA.

The Groups R⁶ and R⁷ when Formula AA is Formula AA-1 and

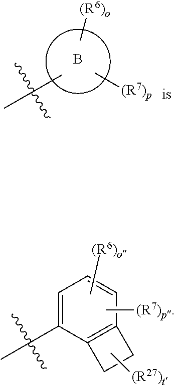

In some embodiments, R⁶ and R⁷, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

The Groups R⁶ and R⁷ when Formula AA is Formula AA-2

In some embodiments, two pairs of R⁶ and R⁷ on adjacent atoms, taken together with the atoms connecting them, independently form two C₄-C₈ carbocyclic rings or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

In some embodiments, two pairs of R⁶ and R⁷ on adjacent atoms, taken together with the atoms connecting them, independently form two C₅ carbocyclic rings optionally independently substituted with one or more R²⁷.

In some embodiments, two pairs of R⁶ and R⁷ on adjacent atoms, taken together with the atoms connecting them, independently form two C₄-C₈ carbocyclic ring or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

In some embodiments, two pairs of R⁶ and R⁷ on adjacent atoms, taken together with the atoms connecting them, independently form two C₅ carbocyclic rings optionally independently substituted with one or more R²⁷.

The Group R⁷ when Formula AA is Formula AA-1 and

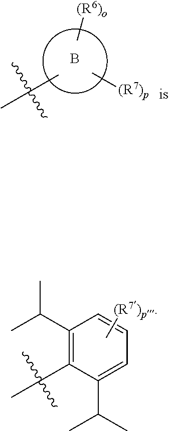

In some embodiments,

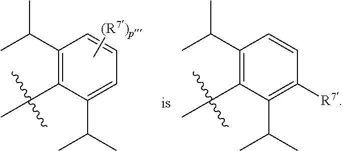

In some embodiments,

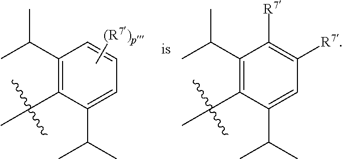

In some embodiments,

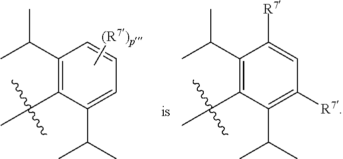

In some embodiments,

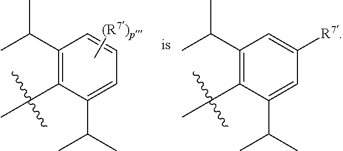

In certain embodiments of the foregoing, R^{7′}, at each occurrence, is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, I, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵.

In certain embodiments of the foregoing, R^{7′}, at each occurrence, is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, I, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, C₃-C₁₀ cycloalkyl optionally substituted with one or more R²⁵, and 3- to 10-membered heterocycloalkyl optionally substituted with one or more R²⁵.

In certain embodiments of the foregoing, R^{7′}, at each occurrence, is independently selected from C₁-C₆ alkyl, C₁-C₆ haloalkyl, C₁-C₆ alkoxy, I, C₁-C₆ haloalkoxy, COC₁-C₆ alkyl, CO₂C₁-C₆ alkyl, CO₂C₃-C₈ cycloalkyl, C₃-C₁₀ cycloalkyl, and 3- to 10-membered heterocycloalkyl.

The Groups R and R when Formula AA is Formula AA-1 and

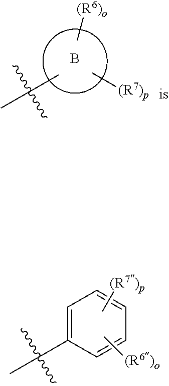

In some embodiments, each occurrence of R^{6″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SC₁-C₆ alkyl optionally substituted with one or more R²⁵, S(O₂)C₂-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵.

In some embodiments, each occurrence of R^{7″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵.

In some embodiments, each occurrence of R^{6″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, and 3- to 10-membered heterocycloalkyl optionally substituted with one or more R²⁵.

In some embodiments, each occurrence of R^{7″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, and 3- to 10-membered heterocycloalkyl optionally substituted with one or more R²⁵.

In some embodiments, each occurrence of R^{6″} is independently selected from C₂ alkyl, C₄-C₆ alkyl optionally substituted with one or more R²⁵, C₁-C₆ haloalkyl optionally substituted with one or more R²⁵, C₂-C₆ alkoxy optionally substituted with one or more R²⁵, C₁-C₆ haloalkoxy optionally substituted with one or more R²⁵, F, Br, I, CN, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, and 3- to 10-membered heterocycloalkyl optionally substituted with one or more R²⁵.

In some embodiments, each occurrence of R^{7″} is independently selected from C₂ alkyl, C₄-C₆ alkyl optionally substituted with one or more R²⁵, C₁-C₆ haloalkyl optionally substituted with one or more R²⁵, C₂-C₆ alkoxy optionally substituted with one or more R²⁵, C₁-C₆ haloalkoxy optionally substituted with one or more R²⁵, halo, CN, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, and 3- to 10-membered heterocycloalkyl optionally substituted with one or more R²⁵.

In some embodiments, at least one R^{6″} is ortho to the bond connecting the B ring to the NH(CO) group of Formula AA.

### The Group R¹⁰

In some embodiments, R¹⁰ is C₁-C₆ alkyl.
In some embodiments, R¹⁰ is methyl.
In some embodiments, R¹⁰ is ethyl.
The groups R⁸ and R⁹
In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to.
In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to.
In some embodiments, each of R⁸ and R⁹ at each occurrence is hydrogen,
In some embodiments, each R⁸ at each occurrence is hydrogen and each R⁹ at each occurrence is C₁-C₆ alkyl.
In some embodiments, each R⁸ at each occurrence is hydrogen and each R⁹ at each occurrence is methyl.
In some embodiments, each R⁸ at each occurrence is hydrogen and each R⁹ at each occurrence is ethyl.
In some embodiments, each of R⁸ and R⁹ at each occurrence is methyl.
In some embodiments, each of R⁸ and R⁹ at each occurrence is ethyl.
In some embodiments, R⁸ and R⁹ taken together with the nitrogen they are attached to form a 3-membered ring.
In some embodiments, R⁸ and R⁹ taken together with the nitrogen they are attached to form a 4-membered ring.
In some embodiments, R⁸ and R⁹ taken together with the nitrogen they are attached to form a 5-membered ring.
In some embodiments, R⁸ and R⁹ taken together with the nitrogen they are attached to form a 6-membered ring optionally containing one or more oxygen atoms in addition to the nitrogen they are attached to.
In some embodiments, R⁸ and R⁹ taken together with the nitrogen they are attached to form a 6-membered ring optionally containing one or more nitrogen atoms in addition to the nitrogen they are attached to.
In some embodiments, R⁸ and R⁹ taken together with the nitrogen they are attached to form a 7-membered ring.
In some embodiments, one of R⁸ and R⁹ is C(O)R¹³.
In some embodiments, one of R⁸ and R⁹ is C(O)₂R¹³.
In certain embodiments of foregoing (when one of R⁸ and R⁹ is C(O)R¹³ or C(0)₂R¹³), the other one of R⁸ and R⁹ is selected from H and C₁₋₆ alkyl.

### The Group R¹³

In some embodiments, R¹³ is C₁-C₆ alkyl.
In some embodiments, R¹³ is methyl.
In some embodiments, R¹³ is ethyl.
In some embodiments, R¹³ is C₆-C₁₀ aryl.
In some embodiments, R¹³ is phenyl.
In some embodiments, R¹³ is 5- to 10-membered heteroaryl.
The groups R¹¹ and R¹²
In some embodiments, each of R¹¹ and R¹² at each occurrence is independently selected from hydrogen and C₁-C₆ alkyl.
In some embodiments, each of R¹¹ and R¹² at each occurrence is hydrogen,
In some embodiments, each R¹¹ at each occurrence is hydrogen; and each R¹² at each occurrence is C₁-C₆ alkyl.
In some embodiments, each R¹¹ at each occurrence is hydrogen; and each R¹² at each occurrence is methyl.
In some embodiments, each R¹¹ at each occurrence is hydrogen; and each R¹² at each occurrence is ethyl.
In some embodiments, each of R¹¹ and R¹² at each occurrence is methyl.
In some embodiments, each of R¹¹ and R¹² at each occurrence is ethyl.

### Non-Limiting Combinations

In some embodiments,

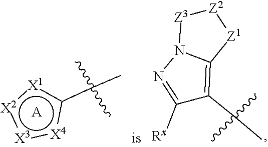

wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).

In some embodiments, ring A is

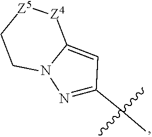

wherein Z⁴ is selected from the group consisting of —CH₂-, —C(O)—, and NH; Z⁵ is selected from the group consisting of O, NH, N—CH₃, and —CH₂-.

In some embodiments, the optionally substituted ring A is selected from the group consisting of:

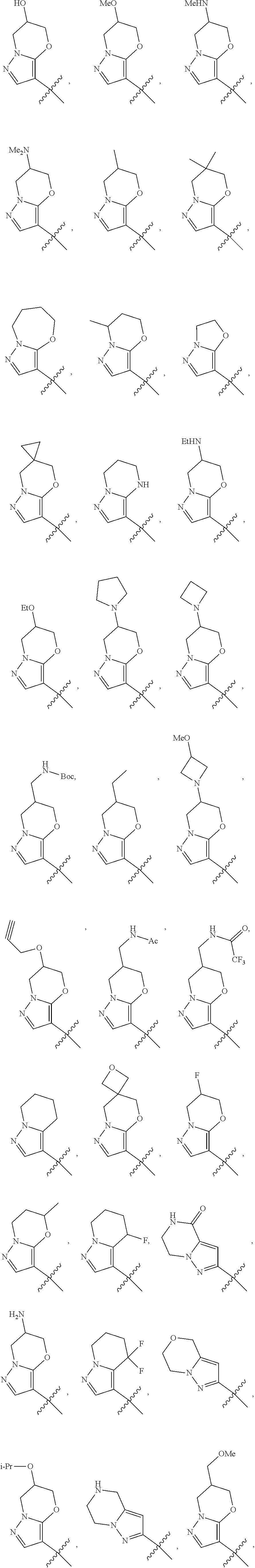

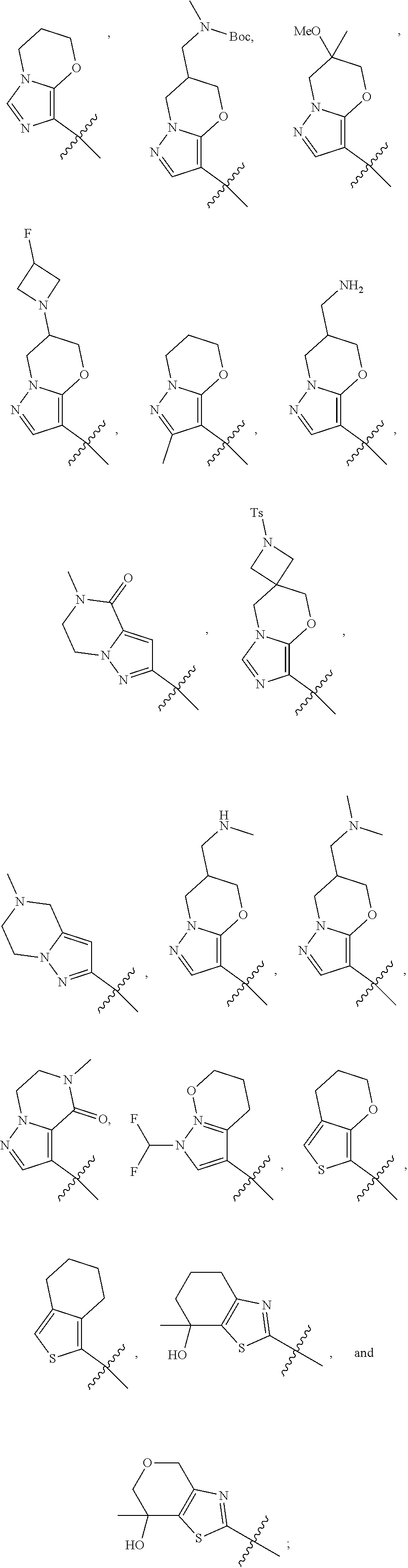

the optionally substituted ring B is selected from the group consisting of:

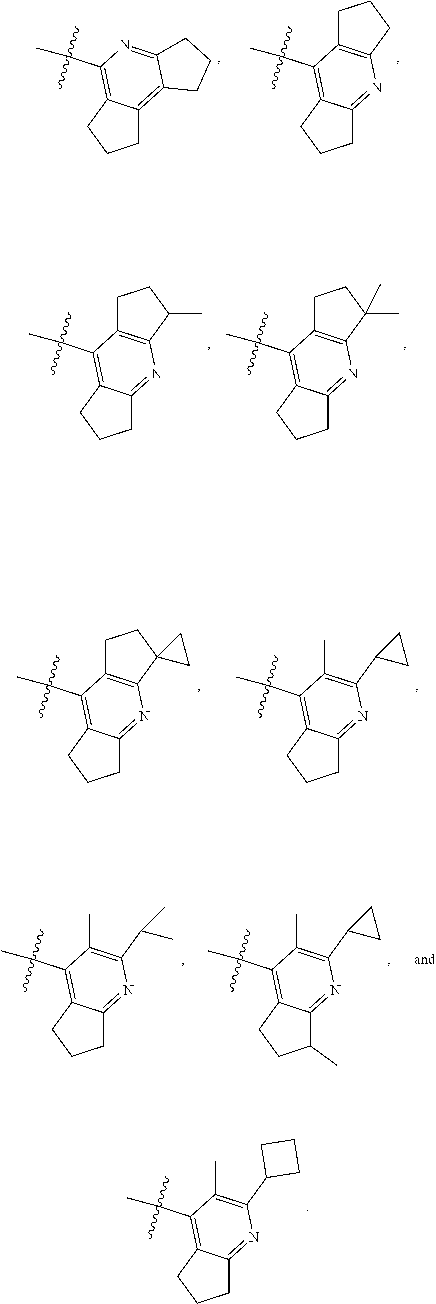

Non-Limiting Combinations when Formula AA is Formula AA-1

In some embodiments, A is

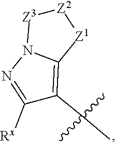

wherein R^{x} is selected from the group consisting of H and C₁-C₆ alkyl (e.g., methyl); Z¹ is selected from the group consisting of O, NH, and —CH₂-optionally substituted with 1-2 R²⁰; Z² is selected from the group consisting of NH and —CH₂-optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂-optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).

In some embodiments, A is

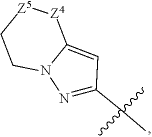

wherein Z⁴ is selected from the group consisting of —CH₂—, —C(O)—, and NH; Z⁵ is selected from the group consisting of O, NH, N—CH₃, and —CH₂-.

In some embodiments the

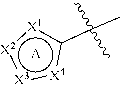

moiety is

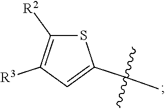

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

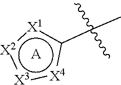

moiety is

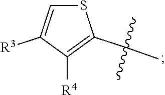

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

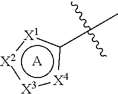

moiety is

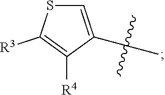

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

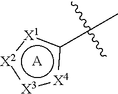

moiety is

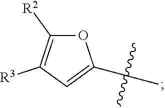

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

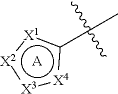

moiety is

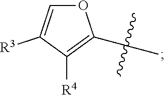

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

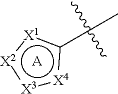

moiety is

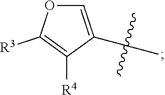

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

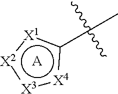

moiety is

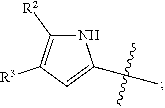

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

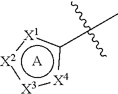

moiety is

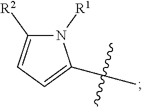

and R¹ and R² taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

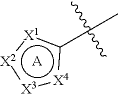

moiety is

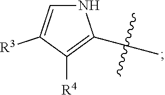

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

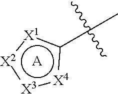

moiety is

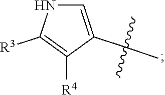

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

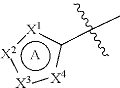

moiety is

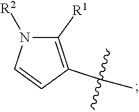

and R¹ and R² taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

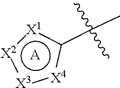

moiety is

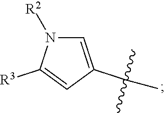

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

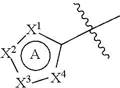

moiety is

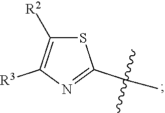

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

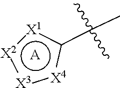

moiety is

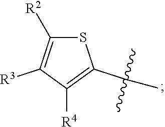

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

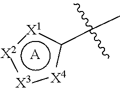

moiety is

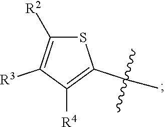

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

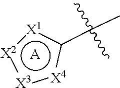

moiety is

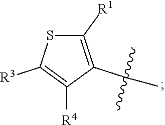

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

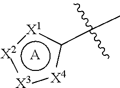

moiety is

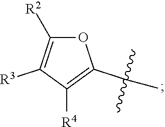

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

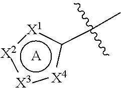

moiety is

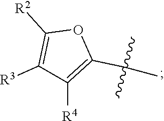

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

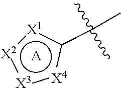

moiety is

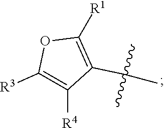

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

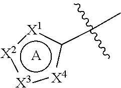

moiety is

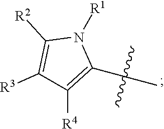

and R¹ and R² taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

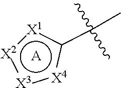

moiety is

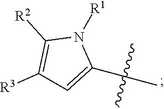

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

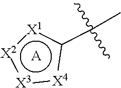

moiety is

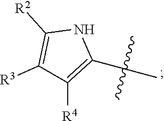

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

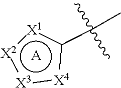

moiety is

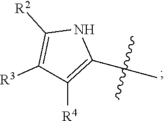

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

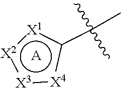

moiety is

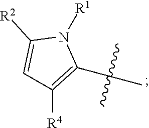

and R¹ and R² taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

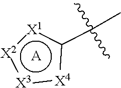

moiety is

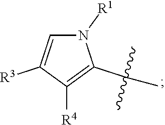

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

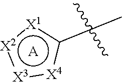

moiety is

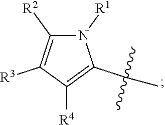

and R¹ and R² taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

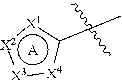

moiety is

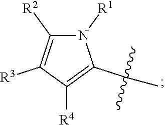

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

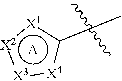

moiety is

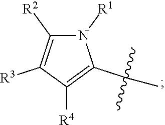

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

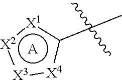

moiety is

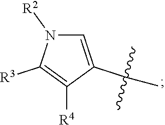

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

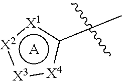

moiety is

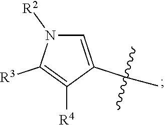

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

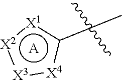

moiety is

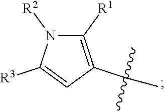

and R¹ and R² taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

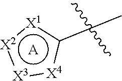

moiety is

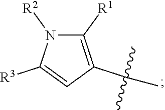

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

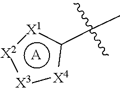

moiety is

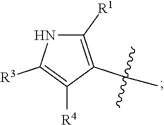

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

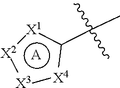

moiety is

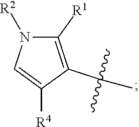

and R¹ and R² taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

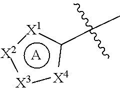

moiety is

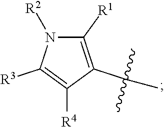

and R¹ and R² and taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

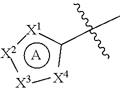

moiety is

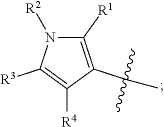

and R² and R³ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments the

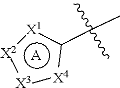

moiety is

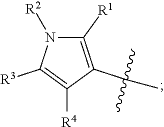

and R³ and R⁴ taken together with the atoms connecting them independently form a ring selected from the group consisting of:

(i) a monocyclic or bicyclic C₄-C₁₂ cycloalkyl ring optionally substituted with one or more R²⁰,

(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,

(iii) a monocyclic or bicyclic C₆-C₁₀ aryl ring optionally substituted with one or more R²⁰, and

(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰.

In some embodiments of the compound of Formula AB,

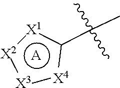

is selected from

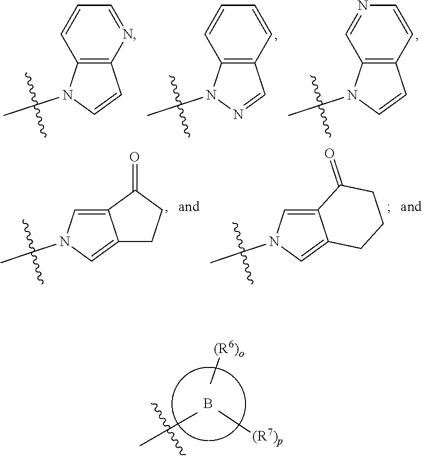

is selected from

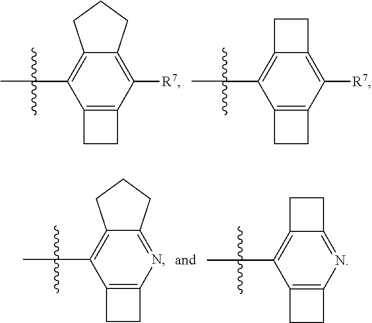

### Additional Features of the Embodiments Herein

In some embodiments, the compound of Formula AA is not

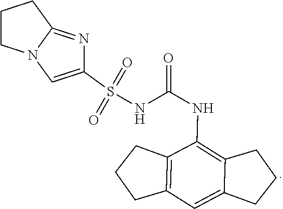 [SMILES: O=C(Nc1c2c(cc3c1CCC3)CCC2)NS(=O)(=O)c1cn2c(n1)CCC2]

In some embodiments, the compound of Formula AA is not

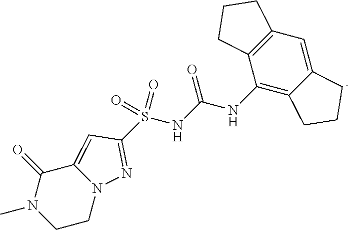 [SMILES: CN1CCn2nc(S(=O)(=O)NC(=O)Nc3c4c(cc5c3CCC5)CCC4)cc2C1=O]

In some embodiments, the optionally substituted ring B in the compound of Formula AA is not selected from the group consisting of:

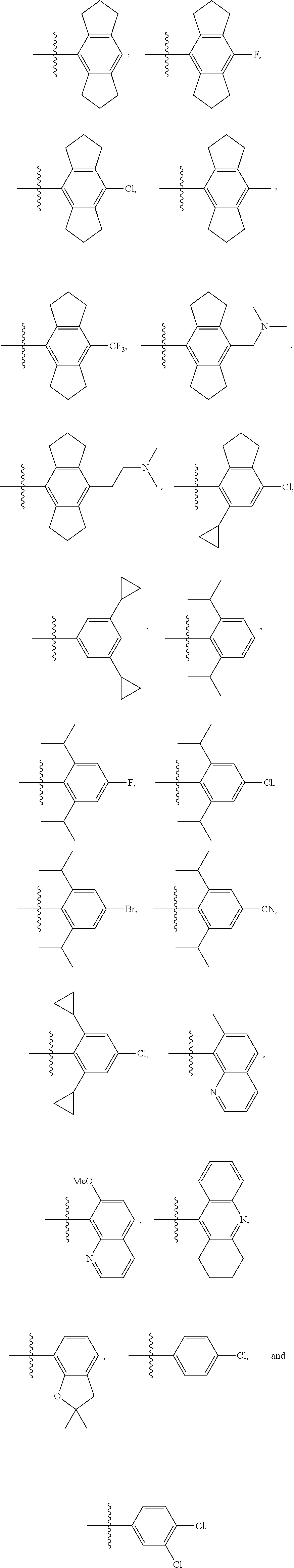

In some embodiments, the compound of Formula AA is not a compound selected from the group consisting of:

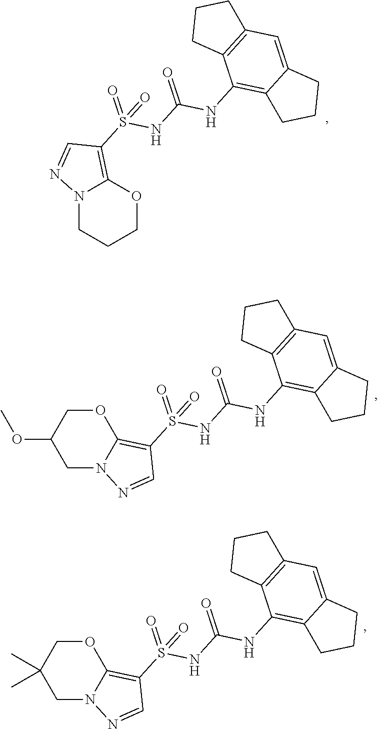 [SMILES: O=C(Nc1c2c(cc3c1CCC3)CCC2)NS(=O)(=O)c1cnn2c1OCCC2 ; "," ; COC1COc2c(S(=O)(=O)NC(=O)Nc3c4c(cc5c3CCC5)CCC4)cnn2C1 ; "," ; CC1(C)COc2c(S(=O)(=O)NC(=O)Nc3c4c(cc5c3CCC5)CCC4)cnn2C1 ; ","]

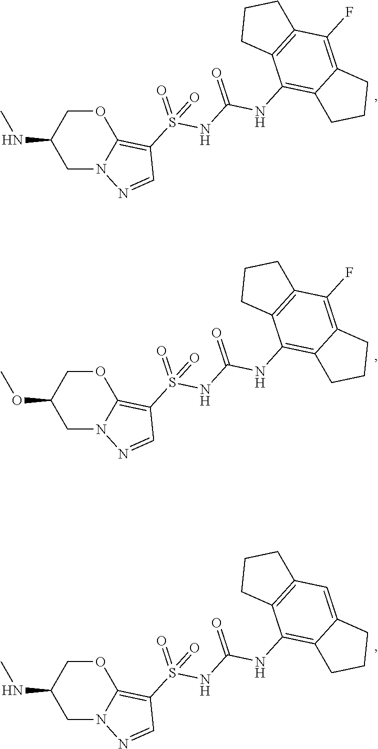 [SMILES: CN[C@H]1COc2c(S(=O)(=O)NC(=O)Nc3c4c(c(F)c5c3CCC5)CCC4)cnn2C1 ; "," ; CO[C@H]1COc2c(S(=O)(=O)NC(=O)Nc3c4c(c(F)c5c3CCC5)CCC4)cnn2C1 ; "," ; CN[C@H]1COc2c(S(=O)(=O)NC(=O)Nc3c4c(cc5c3CCC5)CCC4)cnn2C1 ; ","]

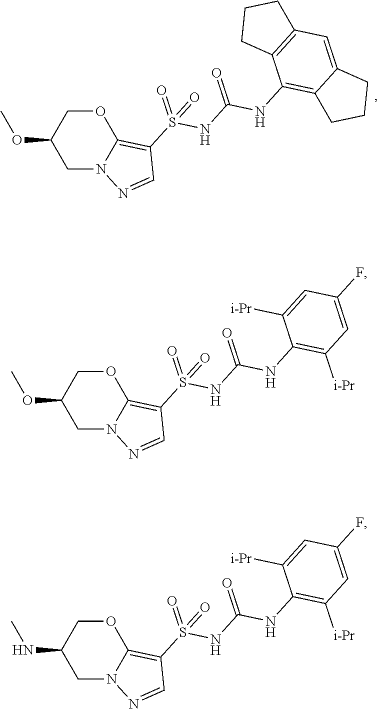 [SMILES: CO[C@H]1COc2c(S(=O)(=O)NC(=O)Nc3c4c(cc5c3CCC5)CCC4)cnn2C1 ; "," ; CO[C@H]1COc2c(S(=O)(=O)NC(=O)Nc3c(C(C)C)cc(F)cc3C(C)C)cnn2C1 ; CN[C@H]1COc2c(S(=O)(=O)NC(=O)Nc3c(C(C)C)cc(F)cc3C(C)C)cnn2C1]

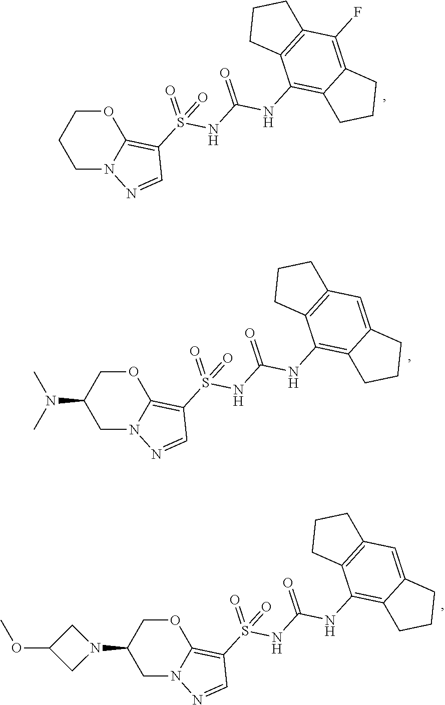 [SMILES: O=C(Nc1c2c(c(F)c3c1CCC3)CCC2)NS(=O)(=O)c1cnn2c1OCCC2 ; "," ; CN(C)[C@H]1COc2c(S(=O)(=O)NC(=O)Nc3c4c(cc5c3CCC5)CCC4)cnn2C1 ; "," ; COC1CN([C@H]2COc3c(S(=O)(=O)NC(=O)Nc4c5c(cc6c4CCC6)CCC5)cnn3C2)C1 ; ","]

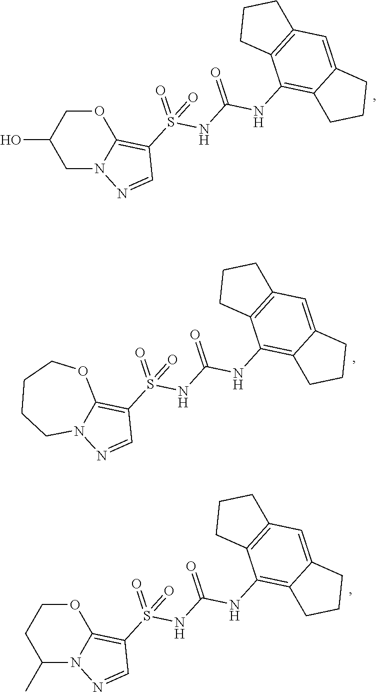 [SMILES: O=C(Nc1c2c(cc3c1CCC3)CCC2)NS(=O)(=O)c1cnn2c1OCC(O)C2 ; "," ; O=C(Nc1c2c(cc3c1CCC3)CCC2)NS(=O)(=O)c1cnn2c1OCCCC2 ; "," ; CC1CCOc2c(S(=O)(=O)NC(=O)Nc3c4c(cc5c3CCC5)CCC4)cnn21 ; ","]

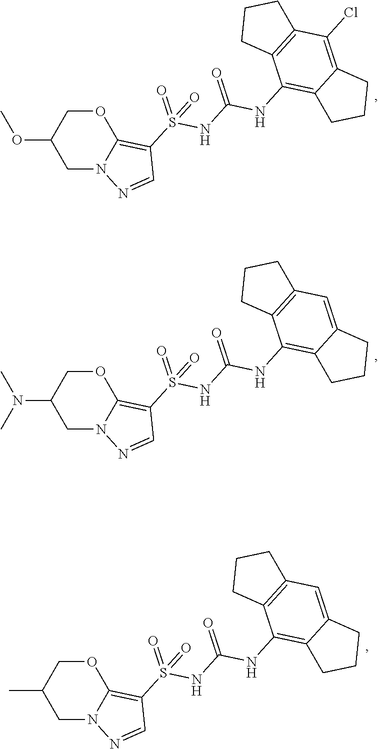 [SMILES: COC1COc2c(S(=O)(=O)NC(=O)Nc3c4c(c(Cl)c5c3CCC5)CCC4)cnn2C1 ; "," ; CN(C)C1COc2c(S(=O)(=O)NC(=O)Nc3c4c(cc5c3CCC5)CCC4)cnn2C1 ; "," ; CC1COc2c(S(=O)(=O)NC(=O)Nc3c4c(cc5c3CCC5)CCC4)cnn2C1 ; ","]

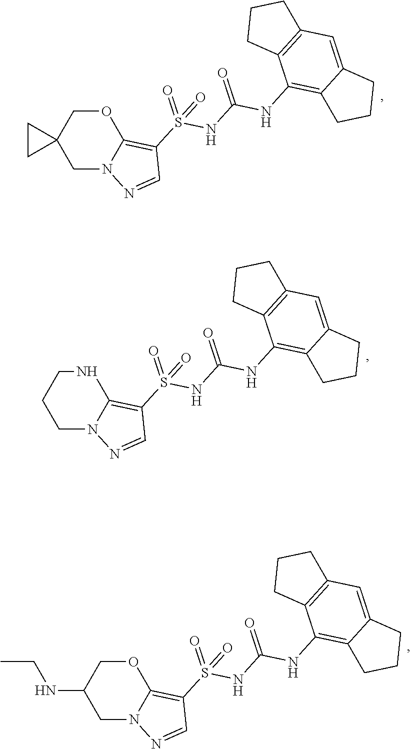 [SMILES: O=C(Nc1c2c(cc3c1CCC3)CCC2)NS(=O)(=O)c1cnn2c1OCC1(CC1)C2 ; "," ; O=C(Nc1c2c(cc3c1CCC3)CCC2)NS(=O)(=O)c1cnn2c1NCCC2 ; "," ; CCNC1COc2c(S(=O)(=O)NC(=O)Nc3c4c(cc5c3CCC5)CCC4)cnn2C1 ; ","]

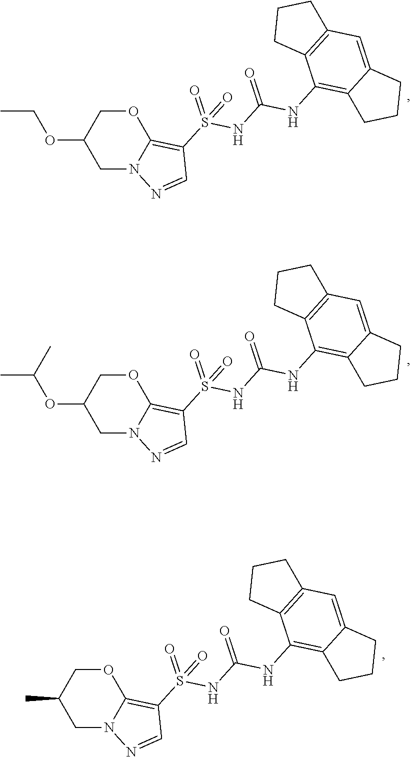 [SMILES: CCOC1COc2c(S(=O)(=O)NC(=O)Nc3c4c(cc5c3CCC5)CCC4)cnn2C1 ; "," ; CC(C)OC1COc2c(S(=O)(=O)NC(=O)Nc3c4c(cc5c3CCC5)CCC4)cnn2C1 ; "," ; C[C@H]1COc2c(S(=O)(=O)NC(=O)Nc3c4c(cc5c3CCC5)CCC4)cnn2C1 ; ","]

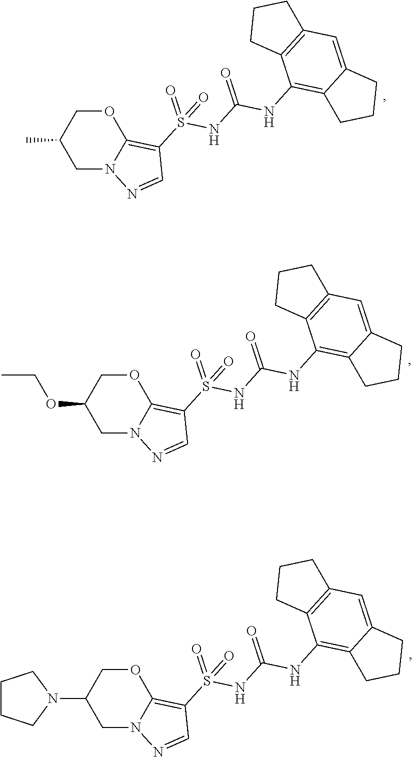 [SMILES: C[C@@H]1COc2c(S(=O)(=O)NC(=O)Nc3c4c(cc5c3CCC5)CCC4)cnn2C1 ; "," ; CCO[C@H]1COc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cnn2c1OCC(N1CCCC1)C2 ; ","]

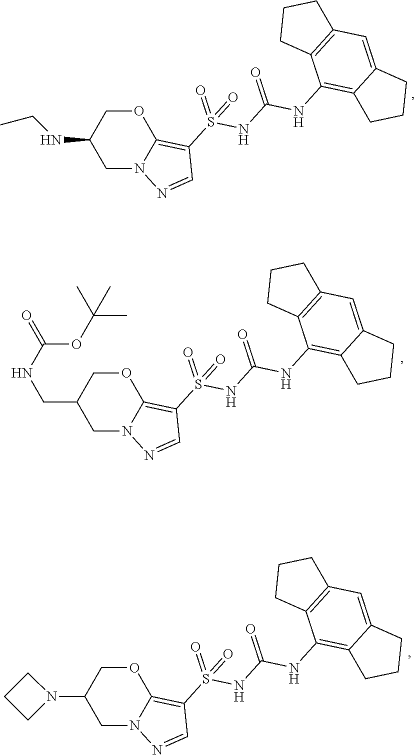 [SMILES: CCN[C@H]1COc2c(S(=O)(=O)NC(=O)Nc3c4c(cc5c3CCC5)CCC4)cnn2C1 ; "," ; CC(C)(C)OC(=O)NCC1COc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cnn2c1OCC(N1CCC1)C2 ; ","]

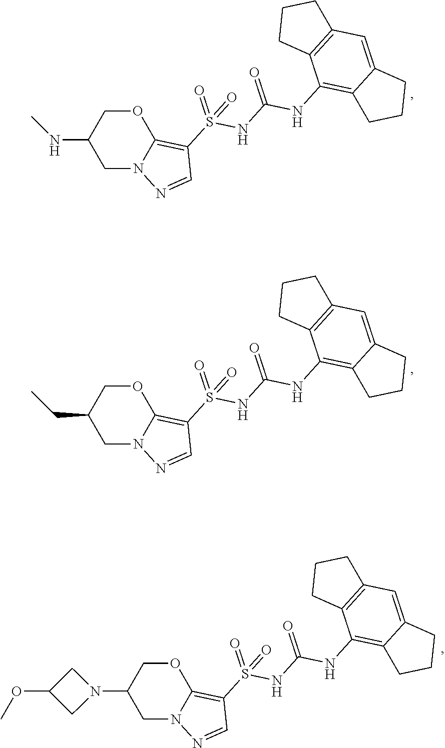 [SMILES: CNC1COc2c(S(=O)(=O)NC(=O)Nc3c4c(cc5c3CCC5)CCC4)cnn2C1 ; "," ; CC[C@H]1COc2c(S(=O)(=O)NC(=O)Nc3c4c(cc5c3CCC5)CCC4)cnn2C1 ; "," ; COC1CN(C2COc3c(S(=O)(=O)NC(=O)Nc4c5c(cc6c4CCC6)CCC5)cnn3C2)C1 ; ","]

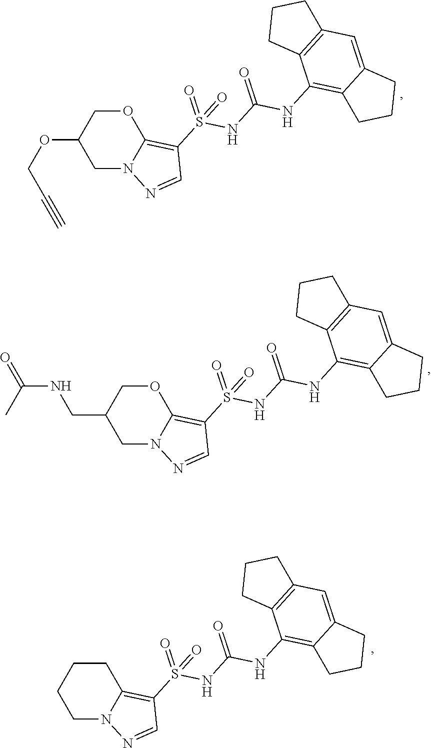 [SMILES: C#CCOC1COc2c(S(=O)(=O)NC(=O)Nc3c4c(cc5c3CCC5)CCC4)cnn2C1 ; "," ; CC(=O)NCC1COc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cnn2c1CCCC2 ; ","]

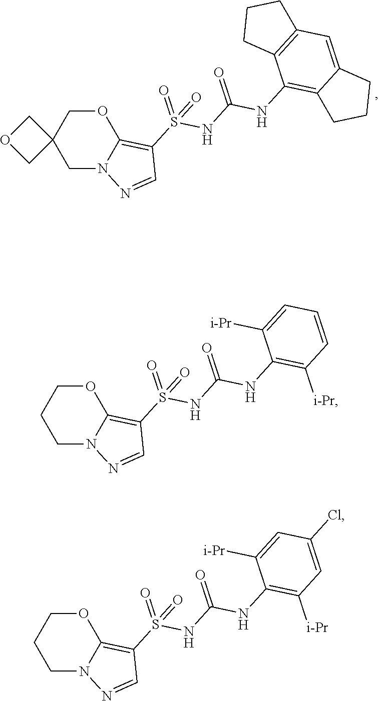 [SMILES: O=C(Nc1c2c(cc3c1CCC3)CCC2)NS(=O)(=O)c1cnn2c1OCC1(COC1)C2 ; "," ; CC(C)c1cccc(C(C)C)c1NC(=O)NS(=O)(=O)c1cnn2c1OCCC2 ; CC(C)c1cc(Cl)cc(C(C)C)c1NC(=O)NS(=O)(=O)c1cnn2c1OCCC2]

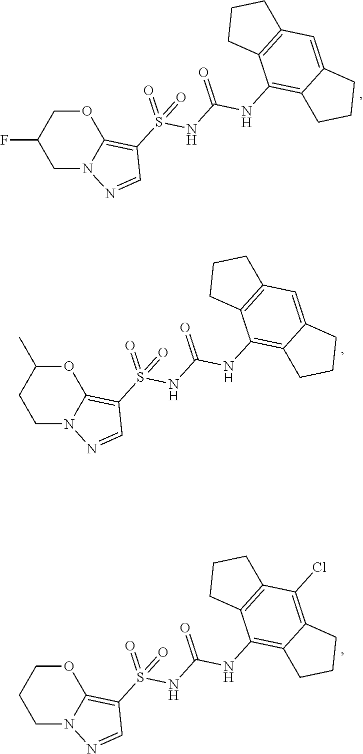 [SMILES: O=C(Nc1c2c(cc3c1CCC3)CCC2)NS(=O)(=O)c1cnn2c1OCC(F)C2 ; "," ; CC1CCn2ncc(S(=O)(=O)NC(=O)Nc3c4c(cc5c3CCC5)CCC4)c2O1 ; "," ; O=C(Nc1c2c(c(Cl)c3c1CCC3)CCC2)NS(=O)(=O)c1cnn2c1OCCC2 ; ","]

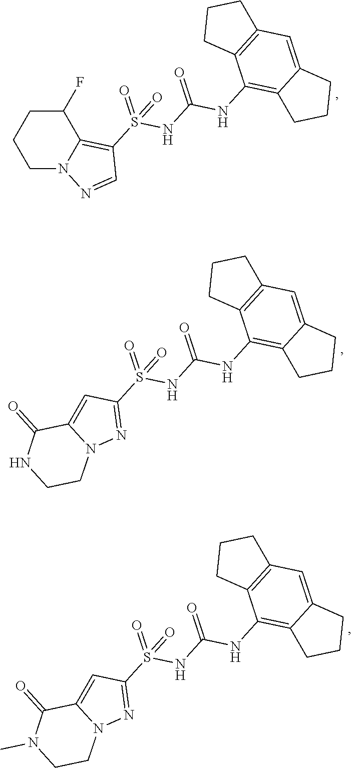 [SMILES: O=C(Nc1c2c(cc3c1CCC3)CCC2)NS(=O)(=O)c1cnn2c1C(F)CCC2 ; "," ; O=C(Nc1c2c(cc3c1CCC3)CCC2)NS(=O)(=O)c1cc2n(n1)CCNC2=O ; "," ; CN1CCn2nc(S(=O)(=O)NC(=O)Nc3c4c(cc5c3CCC5)CCC4)cc2C1=O ; ","]

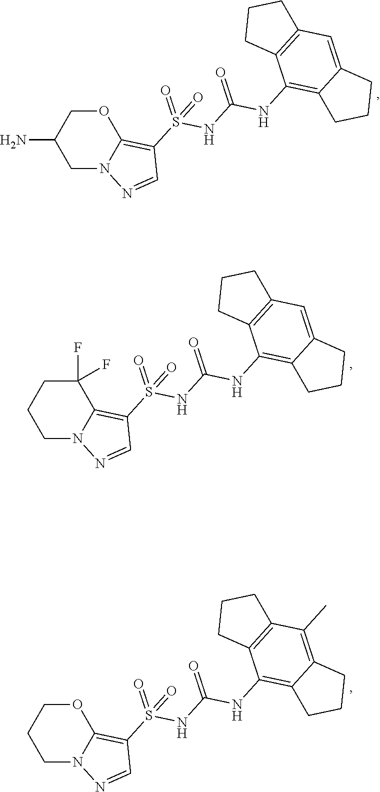 [SMILES: NC1COc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cnn2c1C(F)(F)CCC2 ; "," ; Cc1c2c(c(NC(=O)NS(=O)(=O)c3cnn4c3OCCC4)c3c1CCC3)CCC2 ; ","]

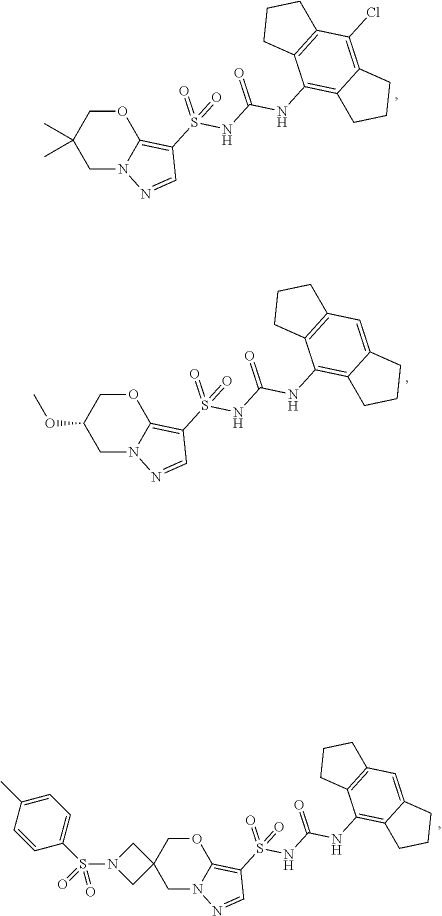 [SMILES: CC1(C)COc2c(S(=O)(=O)NC(=O)Nc3c4c(c(Cl)c5c3CCC5)CCC4)cnn2C1 ; "," ; CO[C@@H]1COc2c(S(=O)(=O)NC(=O)Nc3c4c(cc5c3CCC5)CCC4)cnn2C1 ; "," ; Cc1ccc(S(=O)(=O)N2CC3(COc4c(S(=O)(=O)NC(=O)Nc5c6c(cc7c5CCC7)CCC6)cnn4C3)C2)cc1 ; ","]

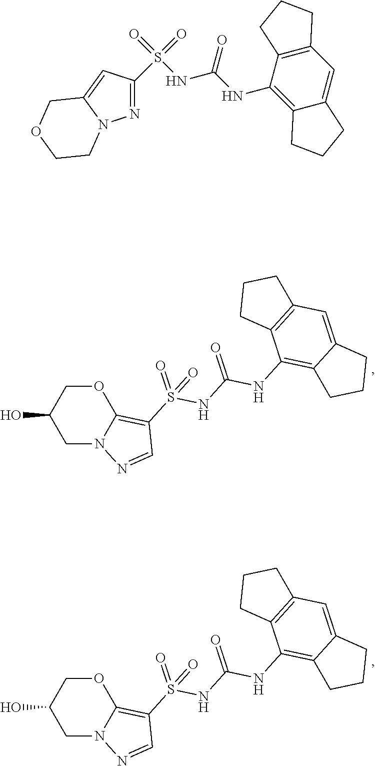 [SMILES: O=C(Nc1c2c(cc3c1CCC3)CCC2)NS(=O)(=O)c1cc2n(n1)CCOC2 ; O=C(Nc1c2c(cc3c1CCC3)CCC2)NS(=O)(=O)c1cnn2c1OC[C@H](O)C2 ; "," ; O=C(Nc1c2c(cc3c1CCC3)CCC2)NS(=O)(=O)c1cnn2c1OC[C@@H](O)C2 ; ","]

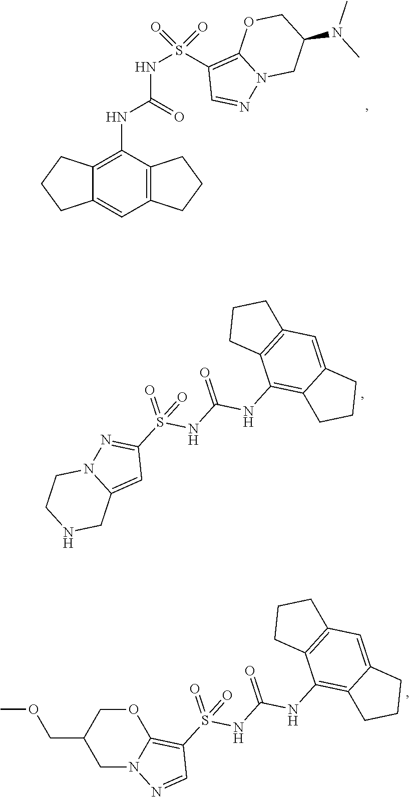

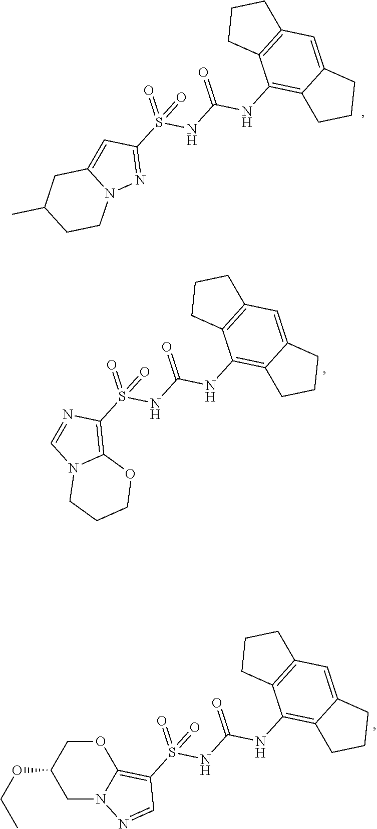 [SMILES: CC1CCn2nc(S(=O)(=O)NC(=O)Nc3c4c(cc5c3CCC5)CCC4)cc2C1 ; "," ; O=C(Nc1c2c(cc3c1CCC3)CCC2)NS(=O)(=O)c1ncn2c1OCCC2 ; "," ; CCO[C@@H]1COc2c(S(=O)(=O)NC(=O)Nc3c4c(cc5c3CCC5)CCC4)cnn2C1 ; ","]

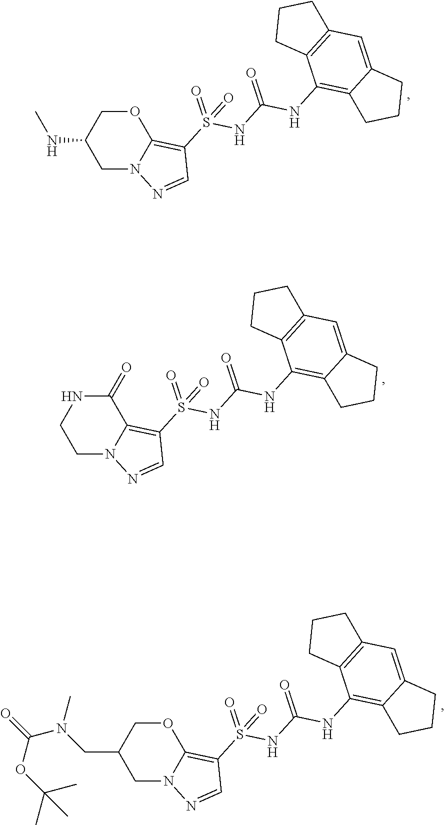 [SMILES: CN[C@@H]1COc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cnn2c1C(=O)NCC2 ; "," ; CN(CC1COc2c(S(=O)(=O)NC(=O)Nc3c4c(cc5c3CCC5)CCC4)cnn2C1)C(=O)OC(C)(C)C ; ","]

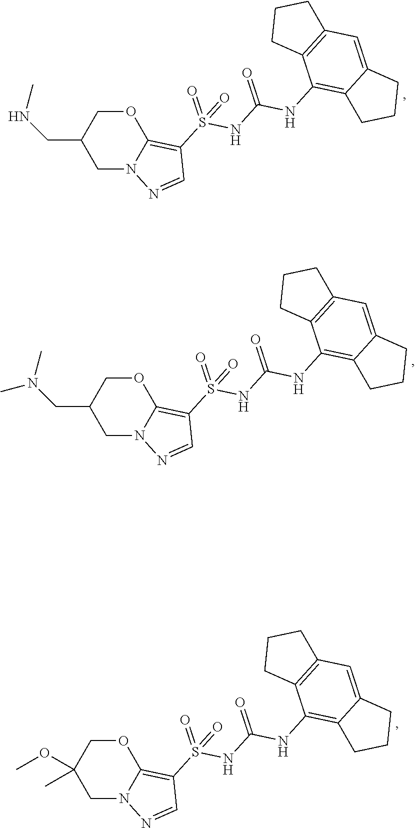 [SMILES: CNCC1COc2c(S(=O)(=O)NC(=O)Nc3c4c(cc5c3CCC5)CCC4)cnn2C1 ; "," ; CN(C)CC1COc2c(S(=O)(=O)NC(=O)Nc3c4c(cc5c3CCC5)CCC4)cnn2C1 ; "," ; COC1(C)COc2c(S(=O)(=O)NC(=O)Nc3c4c(cc5c3CCC5)CCC4)cnn2C1 ; ","]

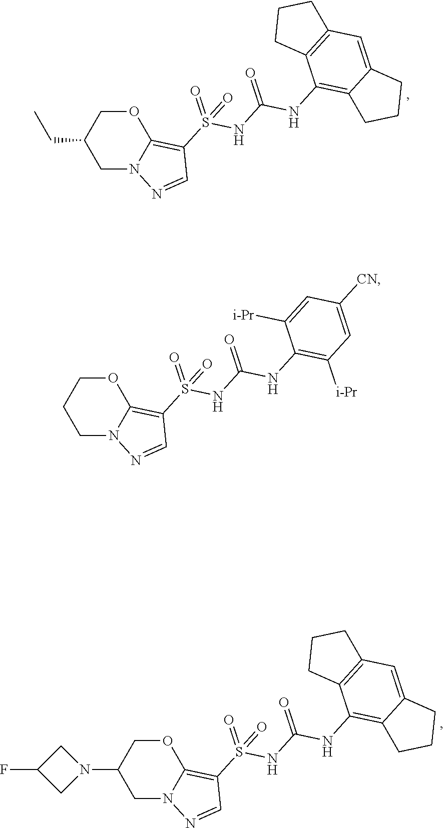 [SMILES: CC[C@@H]1COc2c(S(=O)(=O)NC(=O)Nc3c4c(cc5c3CCC5)CCC4)cnn2C1 ; "," ; CC(C)c1cc(C#N)cc(C(C)C)c1NC(=O)NS(=O)(=O)c1cnn2c1OCCC2 ; O=C(Nc1c2c(cc3c1CCC3)CCC2)NS(=O)(=O)c1cnn2c1OCC(N1CC(F)C1)C2 ; ","]

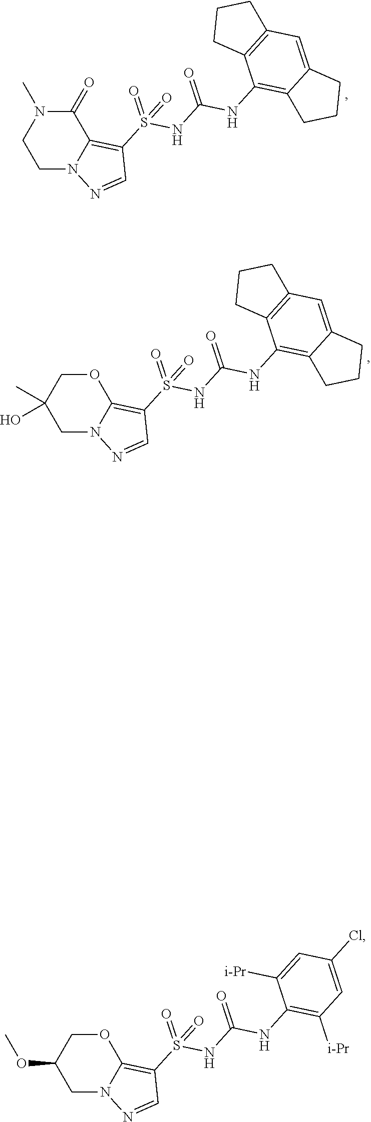 [SMILES: CN1CCn2ncc(S(=O)(=O)NC(=O)Nc3c4c(cc5c3CCC5)CCC4)c2C1=O ; "," ; CC1(O)COc2c(S(=O)(=O)NC(=O)Nc3c4c(cc5c3CCC5)CCC4)cnn2C1 ; "," ; CO[C@H]1COc2c(S(=O)(=O)NC(=O)Nc3c(C(C)C)cc(Cl)cc3C(C)C)cnn2C1]

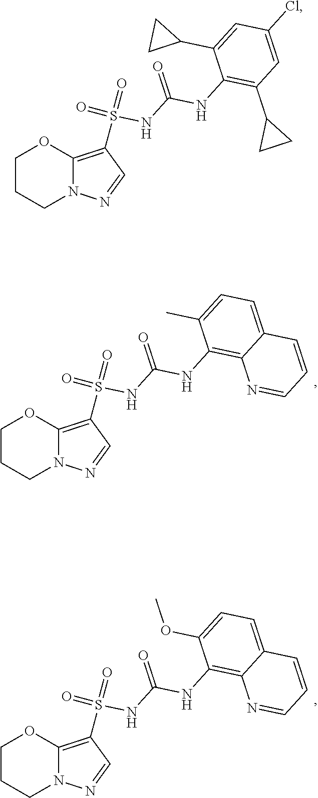 [SMILES: O=C(Nc1c(C2CC2)cc(Cl)cc1C1CC1)NS(=O)(=O)c1cnn2c1OCCC2 ; Cc1ccc2cccnc2c1NC(=O)NS(=O)(=O)c1cnn2c1OCCC2 ; "," ; COc1ccc2cccnc2c1NC(=O)NS(=O)(=O)c1cnn2c1OCCC2 ; ","]

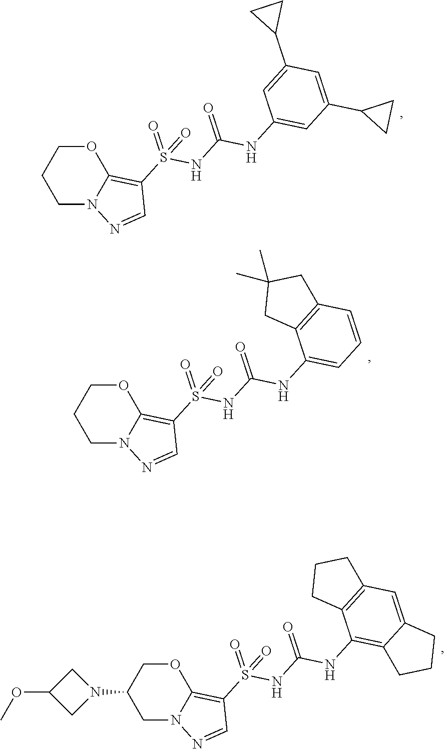 [SMILES: O=C(Nc1cc(C2CC2)cc(C2CC2)c1)NS(=O)(=O)c1cnn2c1OCCC2 ; "," ; CC1(C)Cc2cccc(NC(=O)NS(=O)(=O)c3cnn4c3OCCC4)c2C1 ; "," ; COC1CN([C@@H]2COc3c(S(=O)(=O)NC(=O)Nc4c5c(cc6c4CCC6)CCC5)cnn3C2)C1 ; ","]

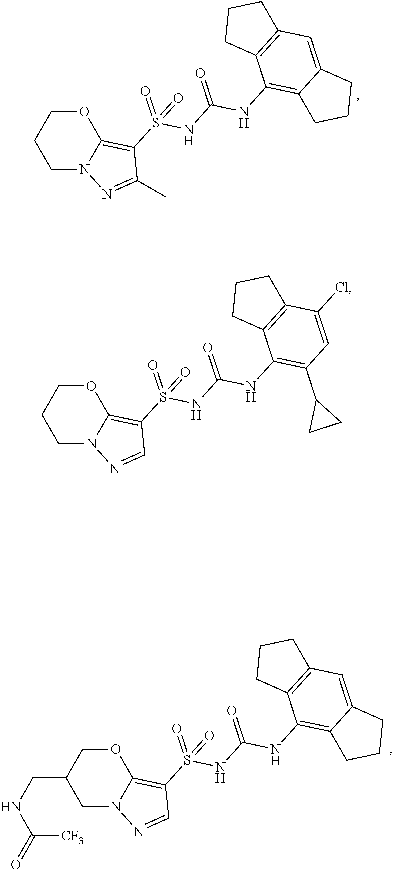 [SMILES: Cc1nn2c(c1S(=O)(=O)NC(=O)Nc1c3c(cc4c1CCC4)CCC3)OCCC2 ; "," ; O=C(Nc1c(C2CC2)cc(Cl)c2c1CCC2)NS(=O)(=O)c1cnn2c1OCCC2 ; O=C(Nc1c2c(cc3c1CCC3)CCC2)NS(=O)(=O)c1cnn2c1OCC(CNC(=O)C(F)(F)F)C2 ; ","]

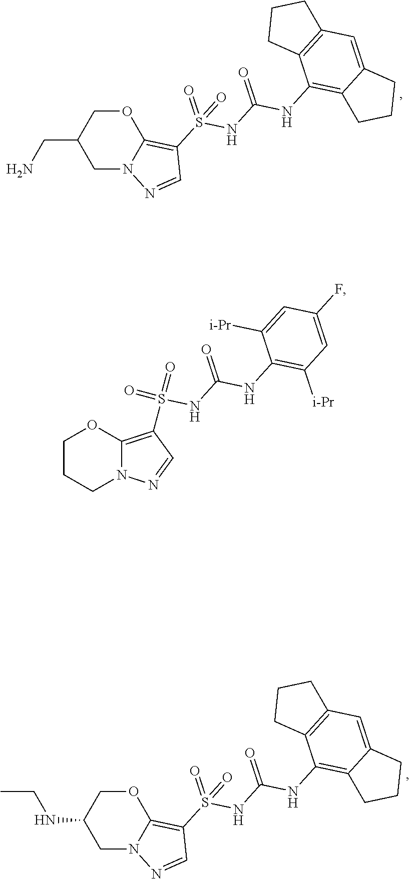 [SMILES: NCC1COc2c(S(=O)(=O)NC(=O)Nc3c4c(cc5c3CCC5)CCC4)cnn2C1 ; "," ; CC(C)c1cc(F)cc(C(C)C)c1NC(=O)NS(=O)(=O)c1cnn2c1OCCC2 ; CCN[C@@H]1COc2c(S(=O)(=O)NC(=O)Nc3c4c(cc5c3CCC5)CCC4)cnn2C1 ; ","]

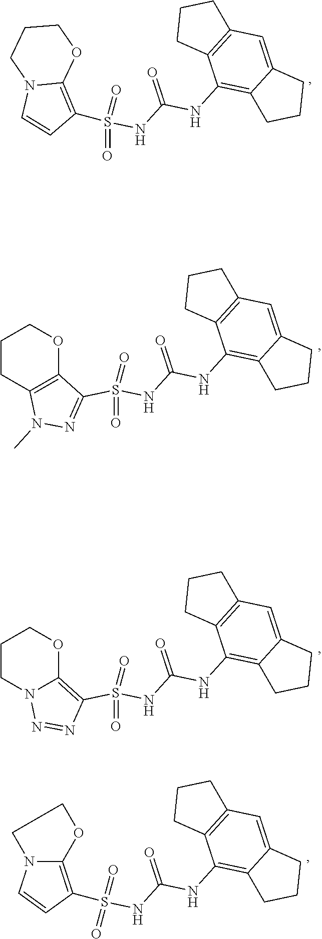

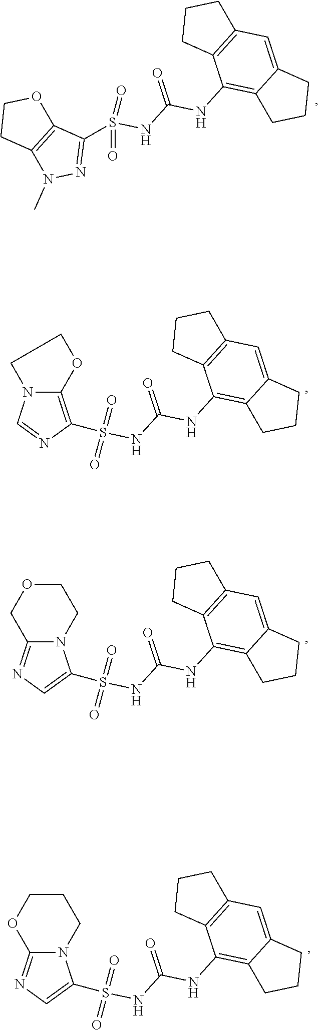

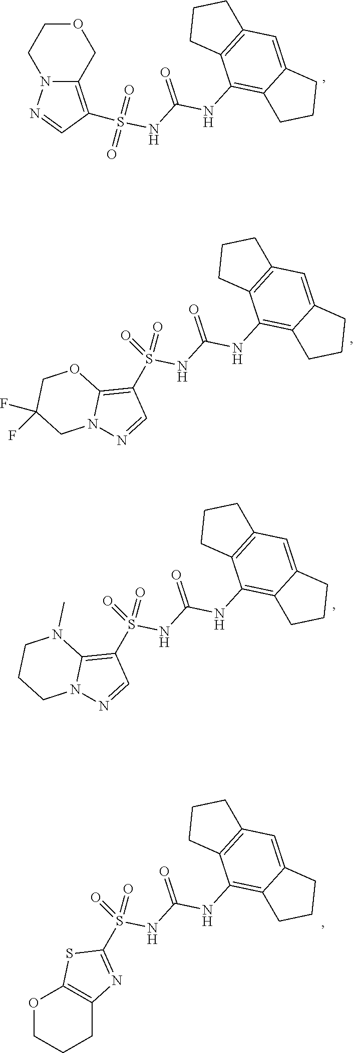

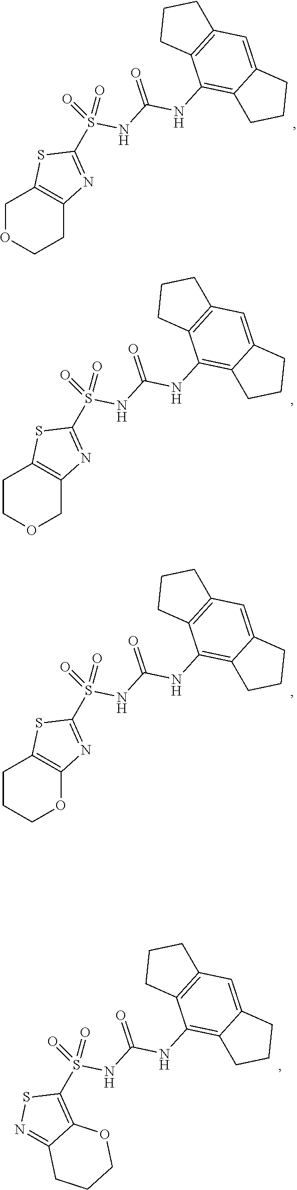

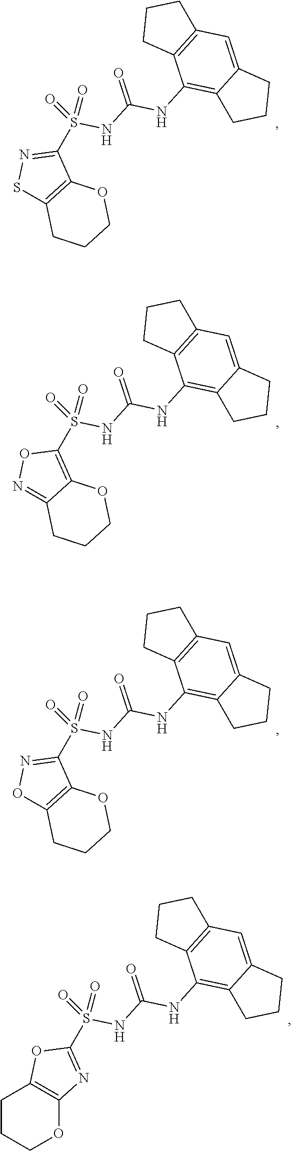

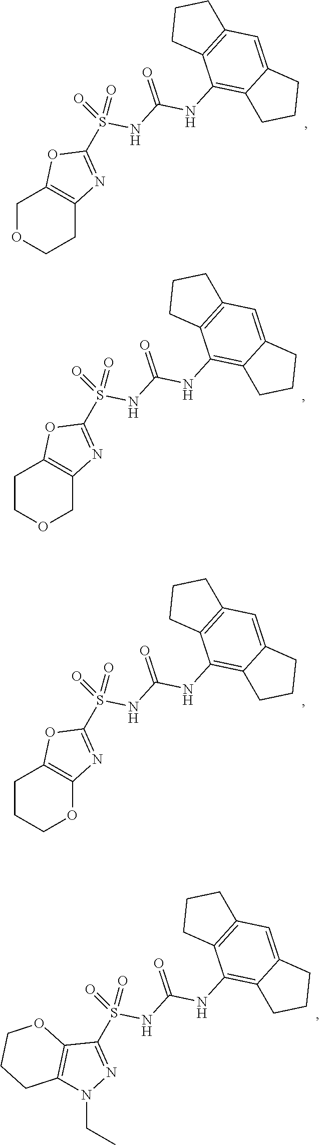

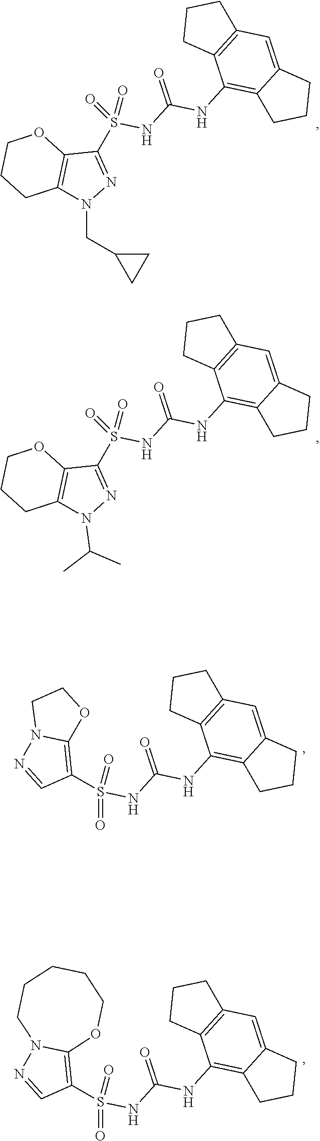

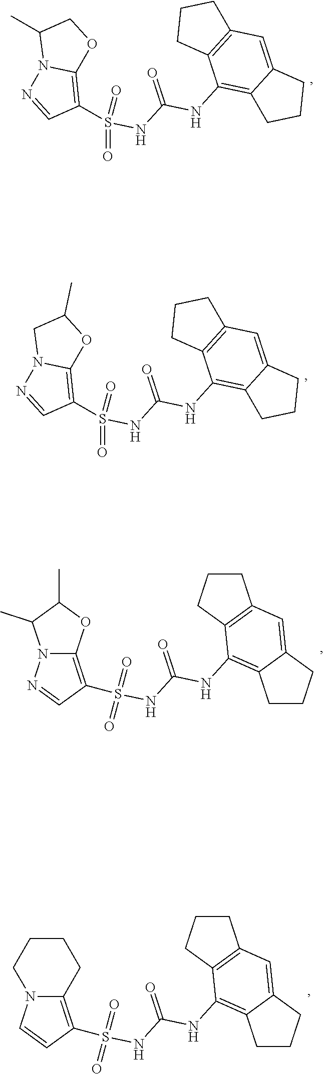

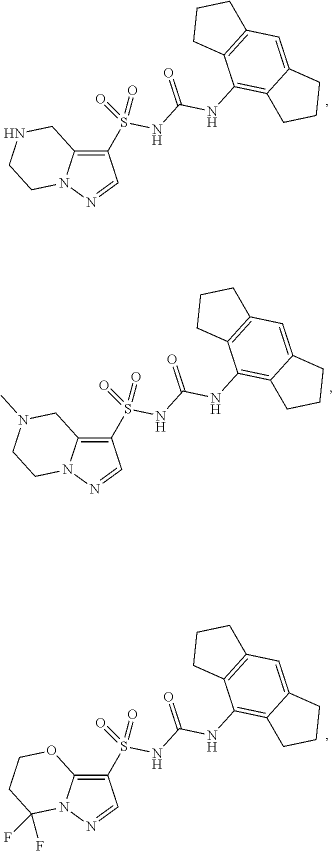 [SMILES: O=C(Nc1c2c(cc3c1CCC3)CCC2)NS(=O)(=O)c1cnn2c1CNCC2 ; "," ; CN1CCn2ncc(S(=O)(=O)NC(=O)Nc3c4c(cc5c3CCC5)CCC4)c2C1 ; "," ; O=C(Nc1c2c(cc3c1CCC3)CCC2)NS(=O)(=O)c1cnn2c1OCCC2(F)F ; ","]

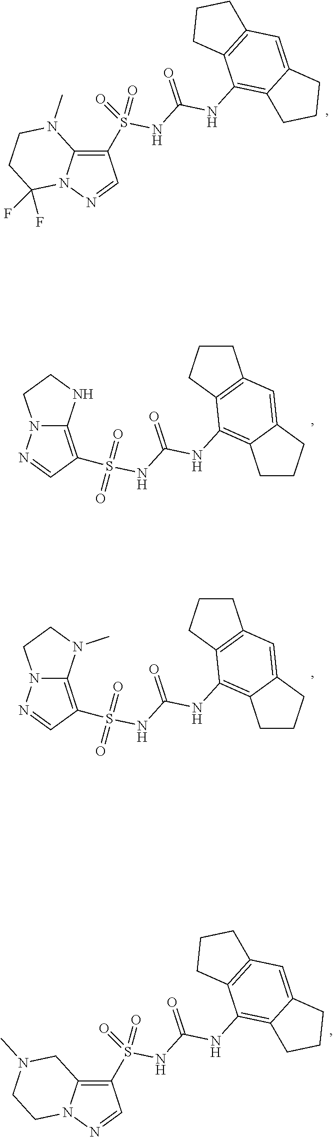

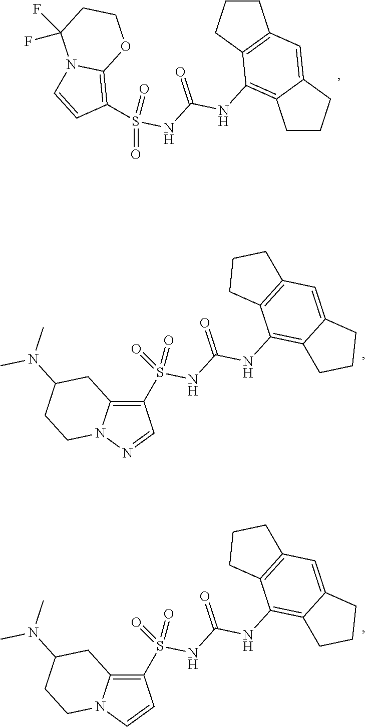 [SMILES: O=C(Nc1c2c(cc3c1CCC3)CCC2)NS(=O)(=O)c1ccn2c1OCCC2(F)F ; "," ; CN(C)C1CCn2ncc(S(=O)(=O)NC(=O)Nc3c4c(cc5c3CCC5)CCC4)c2C1 ; "," ; CN(C)C1CCn2ccc(S(=O)(=O)NC(=O)Nc3c4c(cc5c3CCC5)CCC4)c2C1 ; ","]

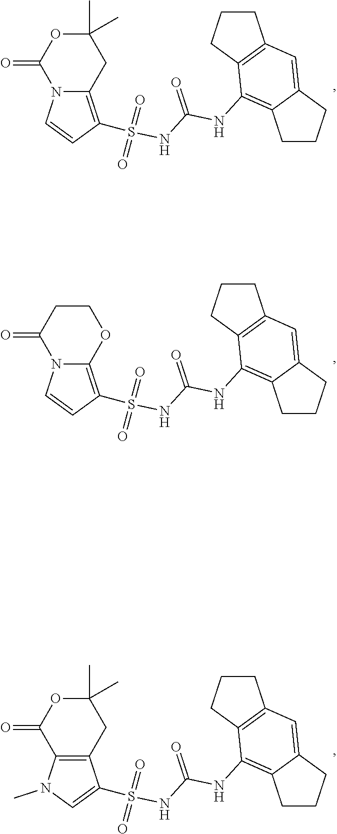 [SMILES: CC1(C)Cc2c(S(=O)(=O)NC(=O)Nc3c4c(cc5c3CCC5)CCC4)ccn2C(=O)O1 ; "," ; O=C(Nc1c2c(cc3c1CCC3)CCC2)NS(=O)(=O)c1ccn2c1OCCC2=O ; "," ; Cn1cc(S(=O)(=O)NC(=O)Nc2c3c(cc4c2CCC4)CCC3)c2c1C(=O)OC(C)(C)C2 ; ","]

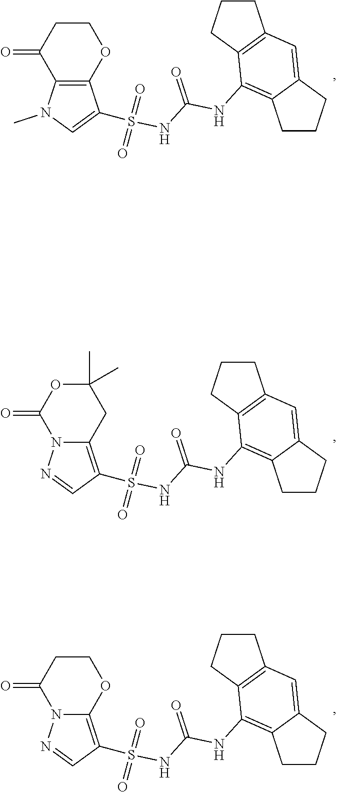 [SMILES: Cn1cc(S(=O)(=O)NC(=O)Nc2c3c(cc4c2CCC4)CCC3)c2c1C(=O)CCO2 ; "," ; CC1(C)Cc2c(S(=O)(=O)NC(=O)Nc3c4c(cc5c3CCC5)CCC4)cnn2C(=O)O1 ; "," ; O=C(Nc1c2c(cc3c1CCC3)CCC2)NS(=O)(=O)c1cnn2c1OCCC2=O ; ","]

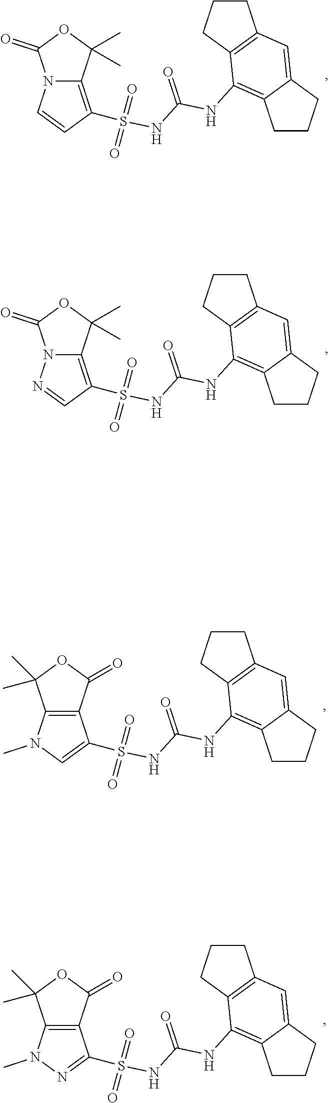

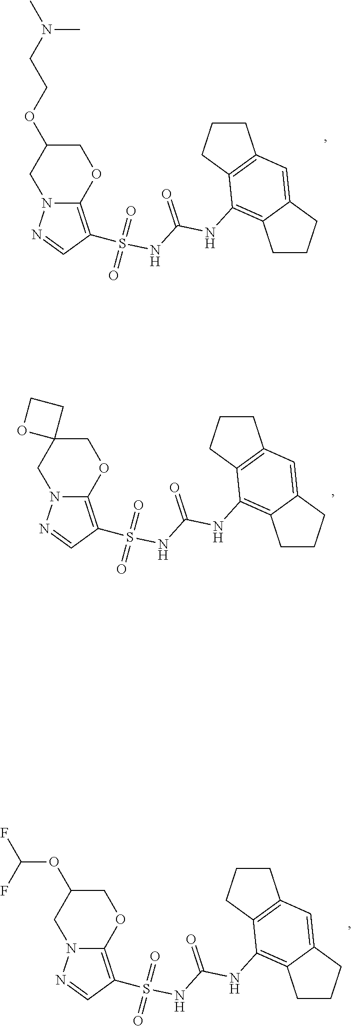 [SMILES: CN(C)CCOC1COc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cnn2c1OCC1(CCO1)C2 ; "," ; O=C(Nc1c2c(cc3c1CCC3)CCC2)NS(=O)(=O)c1cnn2c1OCC(OC(F)F)C2 ; ","]

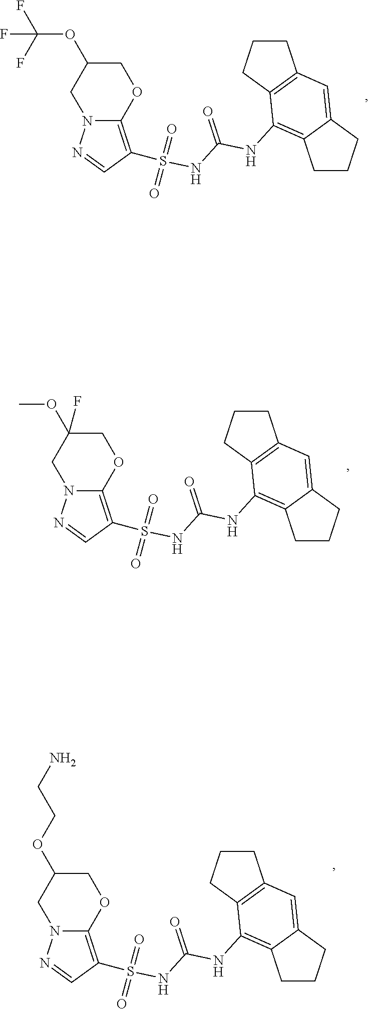 [SMILES: O=C(Nc1c2c(cc3c1CCC3)CCC2)NS(=O)(=O)c1cnn2c1OCC(OC(F)(F)F)C2 ; "," ; COC1(F)COc2c(S(=O)(=O)NC(=O)Nc3c4c(cc5c3CCC5)CCC4)cnn2C1 ; "," ; NCCOC1COc2c(S(=O)(=O)NC(=O)Nc3c4c(cc5c3CCC5)CCC4)cnn2C1 ; ","]

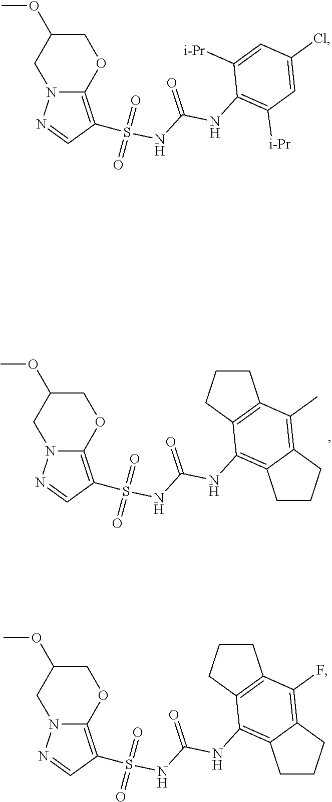 [SMILES: COC1COc2c(S(=O)(=O)NC(=O)Nc3c(C(C)C)cc(Cl)cc3C(C)C)cnn2C1 ; COC1COc2c(S(=O)(=O)NC(=O)Nc3c4c(c(C)c5c3CCC5)CCC4)cnn2C1 ; "," ; COC1COc2c(S(=O)(=O)NC(=O)Nc3c4c(c(F)c5c3CCC5)CCC4)cnn2C1]

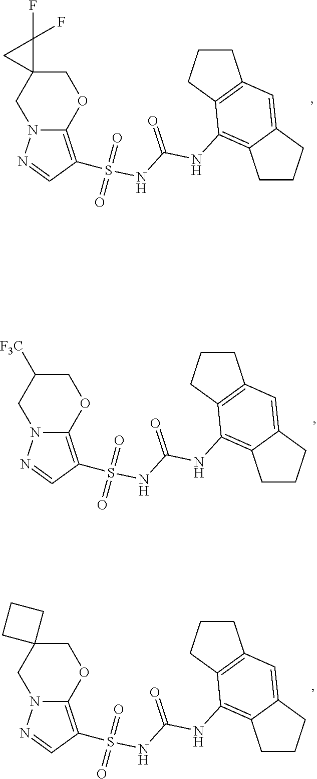 [SMILES: O=C(Nc1c2c(cc3c1CCC3)CCC2)NS(=O)(=O)c1cnn2c1OCC1(C2)CC1(F)F ; "," ; O=C(Nc1c2c(cc3c1CCC3)CCC2)NS(=O)(=O)c1cnn2c1OCC(C(F)(F)F)C2 ; "," ; O=C(Nc1c2c(cc3c1CCC3)CCC2)NS(=O)(=O)c1cnn2c1OCC1(CCC1)C2 ; ","]

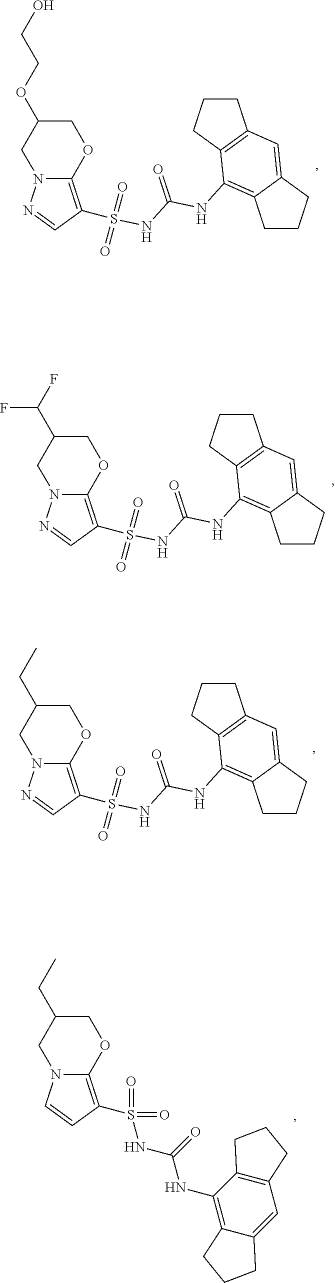

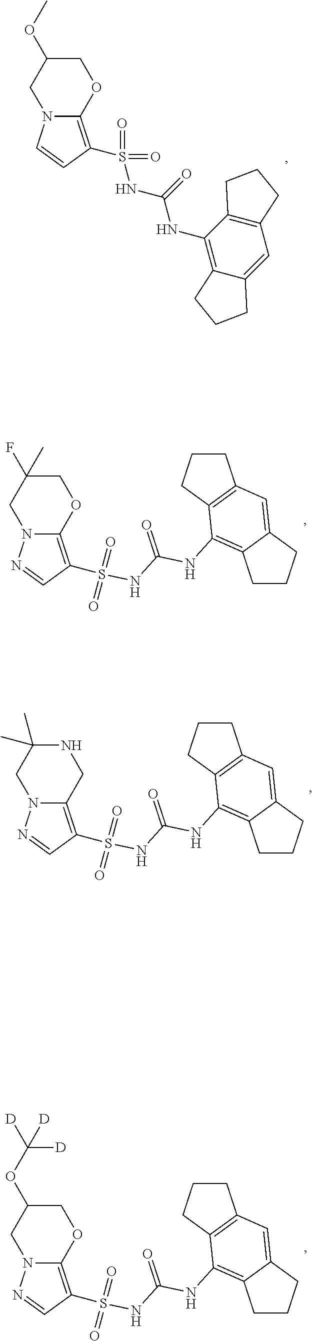

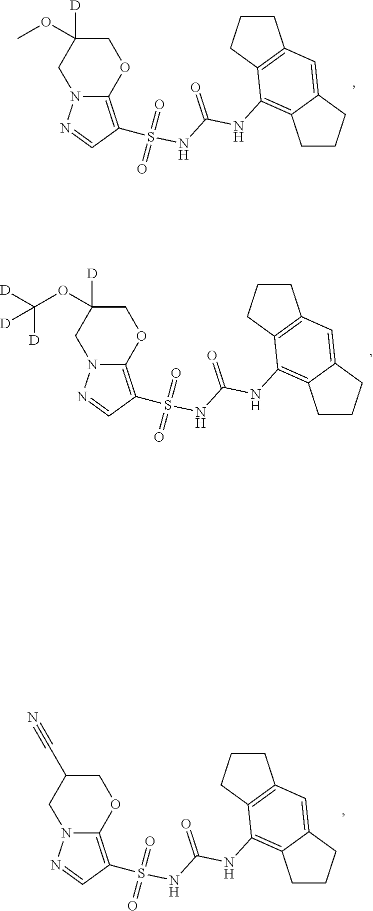 [SMILES: [2H]C1(OC)COc2c(S(=O)(=O)NC(=O)Nc3c4c(cc5c3CCC5)CCC4)cnn2C1 ; "," ; [2H]C([2H])([2H])OC1([2H])COc2c(S(=O)(=O)NC(=O)Nc3c4c(cc5c3CCC5)CCC4)cnn2C1 ; "," ; N#CC1COc2c(S(=O)(=O)NC(=O)Nc3c4c(cc5c3CCC5)CCC4)cnn2C1 ; ","]

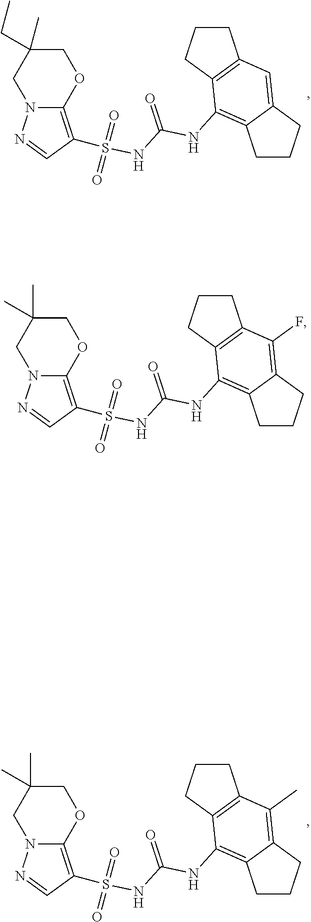 [SMILES: CCC1(C)COc2c(S(=O)(=O)NC(=O)Nc3c4c(cc5c3CCC5)CCC4)cnn2C1 ; "," ; CC1(C)COc2c(S(=O)(=O)NC(=O)Nc3c4c(c(F)c5c3CCC5)CCC4)cnn2C1 ; Cc1c2c(c(NC(=O)NS(=O)(=O)c3cnn4c3OCC(C)(C)C4)c3c1CCC3)CCC2 ; ","]

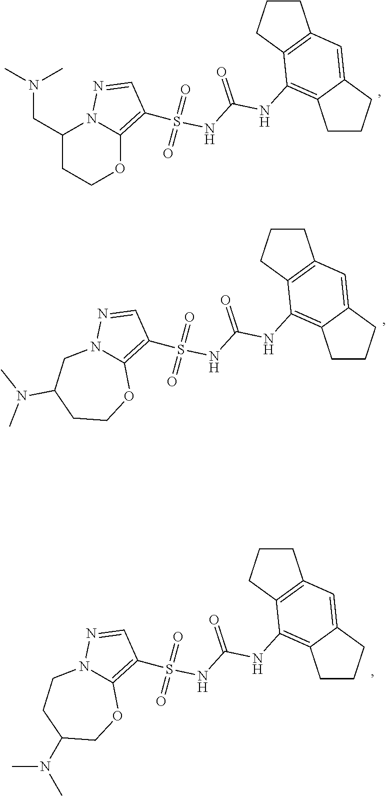 [SMILES: CN(C)CC1CCOc2c(S(=O)(=O)NC(=O)Nc3c4c(cc5c3CCC5)CCC4)cnn21 ; "," ; CN(C)C1CCOc2c(S(=O)(=O)NC(=O)Nc3c4c(cc5c3CCC5)CCC4)cnn2C1 ; "," ; CN(C)C1CCn2ncc(S(=O)(=O)NC(=O)Nc3c4c(cc5c3CCC5)CCC4)c2OC1 ; ","]

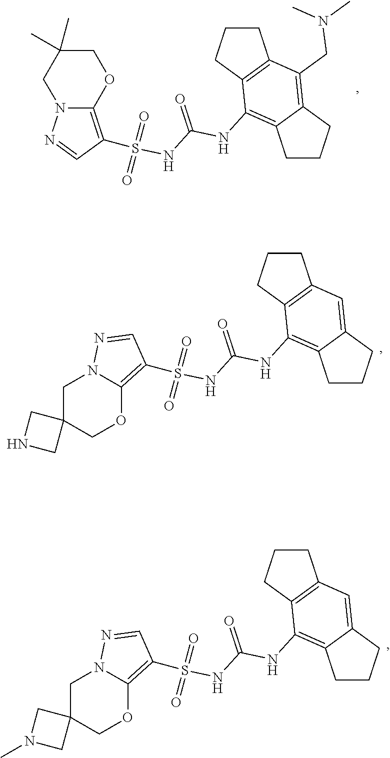

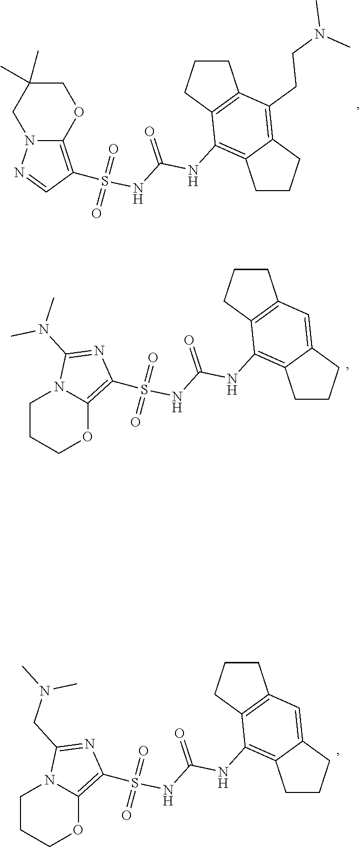

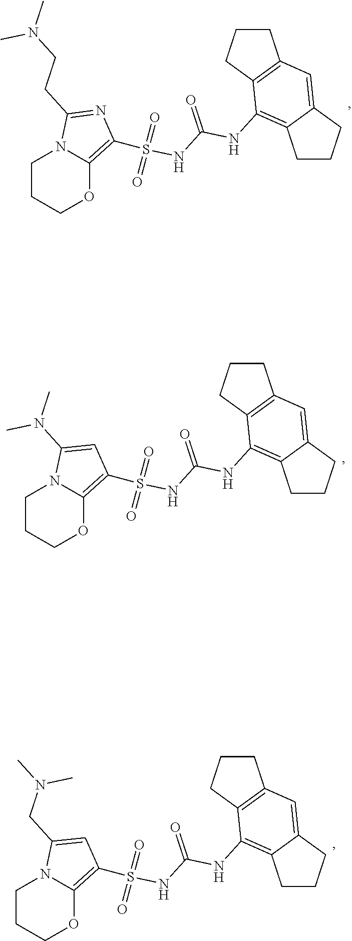 [SMILES: CN(C)CCc1nc(S(=O)(=O)NC(=O)Nc2c3c(cc4c2CCC4)CCC3)c2n1CCCO2 ; "," ; CN(C)c1cc(S(=O)(=O)NC(=O)Nc2c3c(cc4c2CCC4)CCC3)c2n1CCCO2 ; "," ; CN(C)Cc1cc(S(=O)(=O)NC(=O)Nc2c3c(cc4c2CCC4)CCC3)c2n1CCCO2 ; ","]

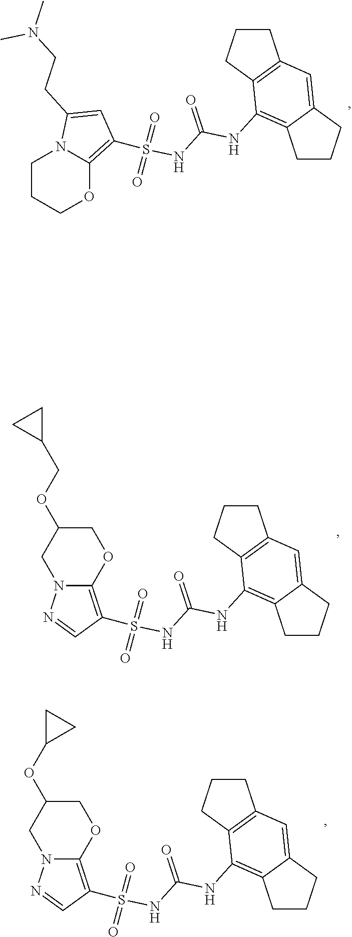 [SMILES: CN(C)CCc1cc(S(=O)(=O)NC(=O)Nc2c3c(cc4c2CCC4)CCC3)c2n1CCCO2 ; "," ; O=C(Nc1c2c(cc3c1CCC3)CCC2)NS(=O)(=O)c1cnn2c1OCC(OCC1CC1)C2 ; "," ; O=C(Nc1c2c(cc3c1CCC3)CCC2)NS(=O)(=O)c1cnn2c1OCC(OC1CC1)C2 ; ","]

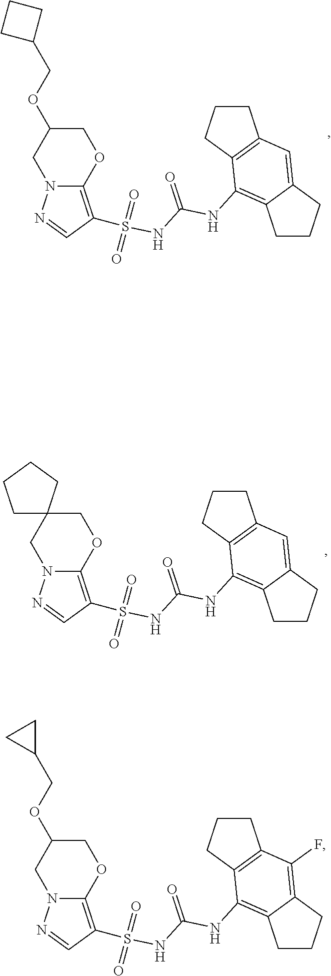 [SMILES: O=C(Nc1c2c(cc3c1CCC3)CCC2)NS(=O)(=O)c1cnn2c1OCC(OCC1CCC1)C2 ; "," ; O=C(Nc1c2c(cc3c1CCC3)CCC2)NS(=O)(=O)c1cnn2c1OCC1(CCCC1)C2 ; "," ; O=C(Nc1c2c(c(F)c3c1CCC3)CCC2)NS(=O)(=O)c1cnn2c1OCC(OCC1CC1)C2]

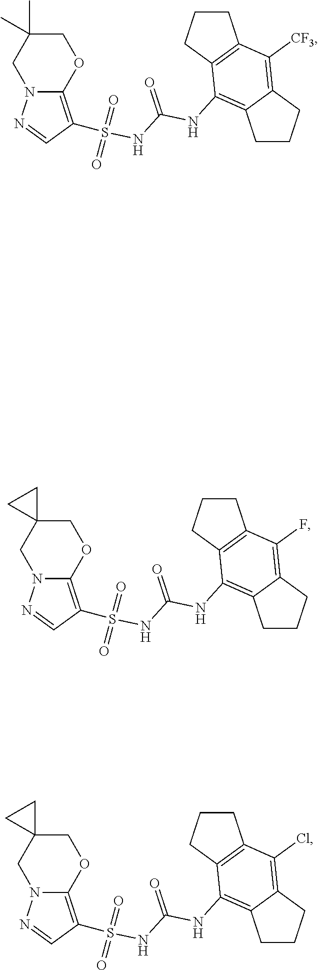 [SMILES: CC1(C)COc2c(S(=O)(=O)NC(=O)Nc3c4c(c(C(F)(F)F)c5c3CCC5)CCC4)cnn2C1 ; O=C(Nc1c2c(c(F)c3c1CCC3)CCC2)NS(=O)(=O)c1cnn2c1OCC1(CC1)C2 ; O=C(Nc1c2c(c(Cl)c3c1CCC3)CCC2)NS(=O)(=O)c1cnn2c1OCC1(CC1)C2]

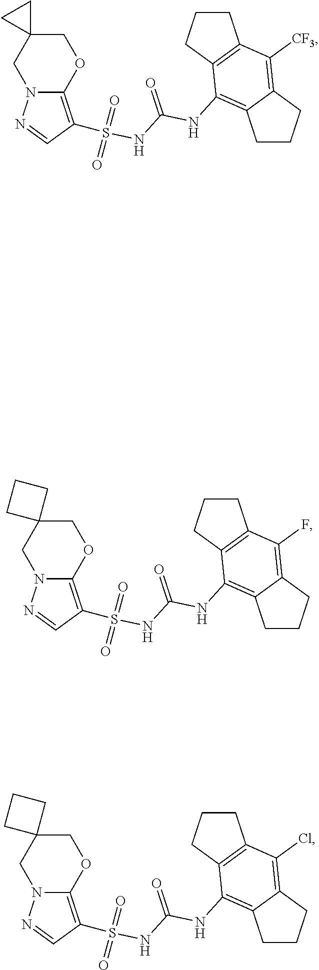 [SMILES: O=C(Nc1c2c(c(C(F)(F)F)c3c1CCC3)CCC2)NS(=O)(=O)c1cnn2c1OCC1(CC1)C2 ; O=C(Nc1c2c(c(F)c3c1CCC3)CCC2)NS(=O)(=O)c1cnn2c1OCC1(CCC1)C2 ; O=C(Nc1c2c(c(Cl)c3c1CCC3)CCC2)NS(=O)(=O)c1cnn2c1OCC1(CCC1)C2]

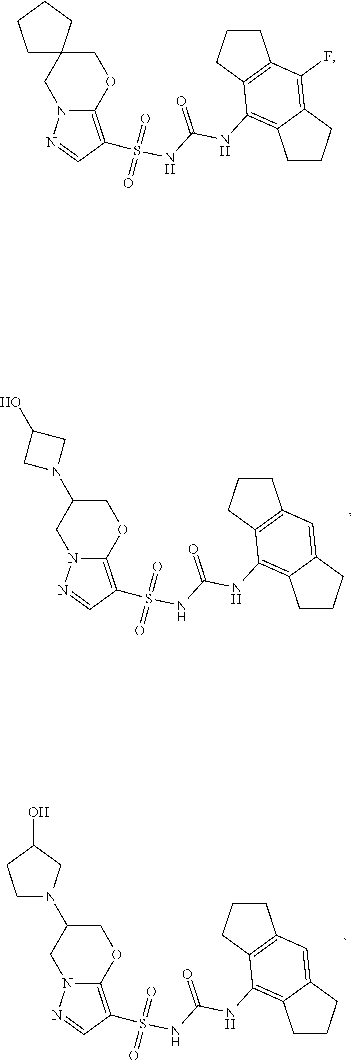 [SMILES: O=C(Nc1c2c(c(F)c3c1CCC3)CCC2)NS(=O)(=O)c1cnn2c1OCC1(CCCC1)C2 ; O=C(Nc1c2c(cc3c1CCC3)CCC2)NS(=O)(=O)c1cnn2c1OCC(N1CC(O)C1)C2 ; "," ; O=C(Nc1c2c(cc3c1CCC3)CCC2)NS(=O)(=O)c1cnn2c1OCC(N1CCC(O)C1)C2 ; ","]

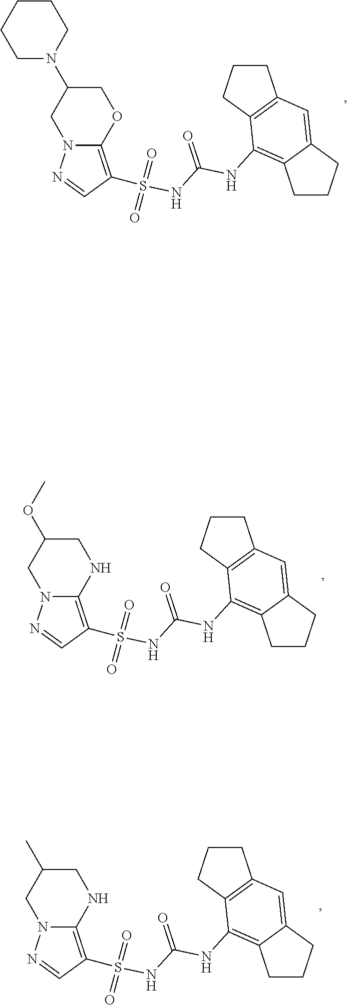 [SMILES: O=C(Nc1c2c(cc3c1CCC3)CCC2)NS(=O)(=O)c1cnn2c1OCC(N1CCCCC1)C2 ; "," ; COC1CNc2c(S(=O)(=O)NC(=O)Nc3c4c(cc5c3CCC5)CCC4)cnn2C1 ; "," ; CC1CNc2c(S(=O)(=O)NC(=O)Nc3c4c(cc5c3CCC5)CCC4)cnn2C1 ; ","]

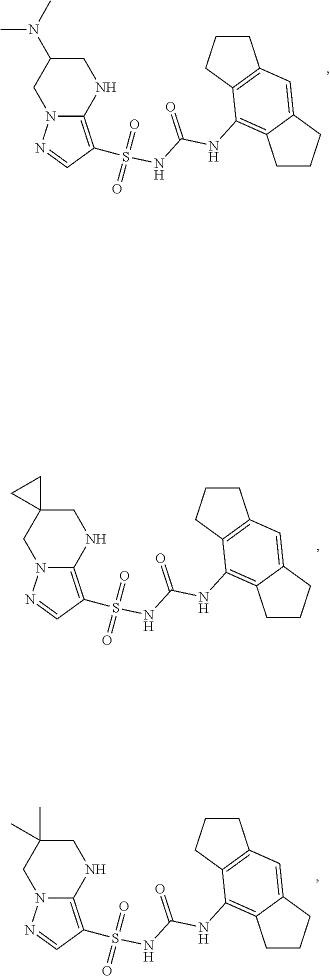 [SMILES: CN(C)C1CNc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cnn2c1NCC1(CC1)C2 ; "," ; CC1(C)CNc2c(S(=O)(=O)NC(=O)Nc3c4c(cc5c3CCC5)CCC4)cnn2C1 ; ","]

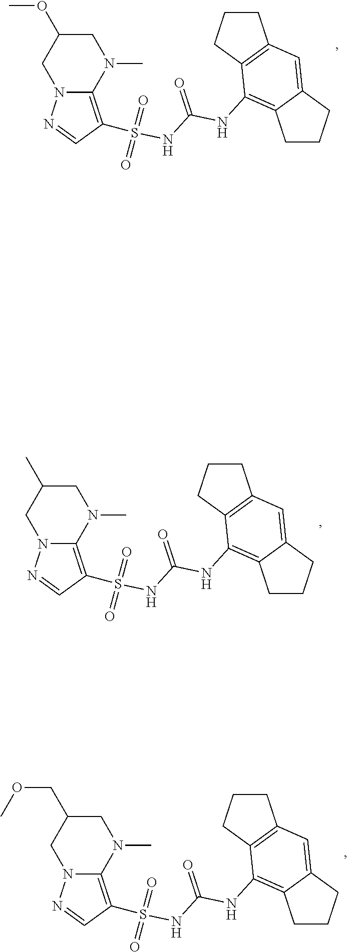 [SMILES: COC1CN(C)c2c(S(=O)(=O)NC(=O)Nc3c4c(cc5c3CCC5)CCC4)cnn2C1 ; "," ; CC1CN(C)c2c(S(=O)(=O)NC(=O)Nc3c4c(cc5c3CCC5)CCC4)cnn2C1 ; "," ; COCC1CN(C)c2c(S(=O)(=O)NC(=O)Nc3c4c(cc5c3CCC5)CCC4)cnn2C1 ; ","]

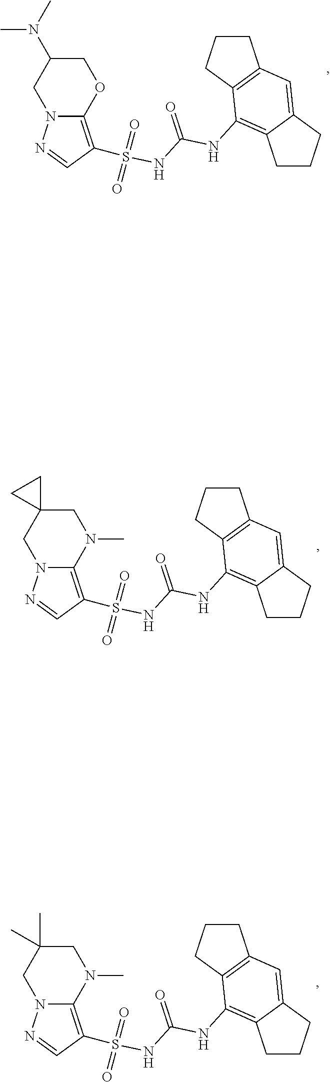 [SMILES: CN(C)C1COc2c(S(=O)(=O)NC(=O)Nc3c4c(cc5c3CCC5)CCC4)cnn2C1 ; "," ; CN1CC2(CC2)Cn2ncc(S(=O)(=O)NC(=O)Nc3c4c(cc5c3CCC5)CCC4)c21 ; "," ; CN1CC(C)(C)Cn2ncc(S(=O)(=O)NC(=O)Nc3c4c(cc5c3CCC5)CCC4)c21 ; ","]

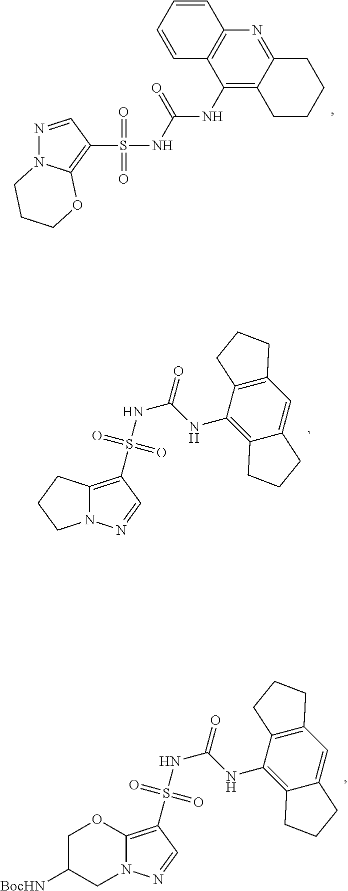 [SMILES: O=C(Nc1c2c(nc3ccccc13)CCCC2)NS(=O)(=O)c1cnn2c1OCCC2 ; "," ; O=C(Nc1c2c(cc3c1CCC3)CCC2)NS(=O)(=O)c1cnn2c1CCC2 ; "," ; CC(C)(C)OC(=O)NC1COc2c(S(=O)(=O)NC(=O)Nc3c4c(cc5c3CCC5)CCC4)cnn2C1 ; ","]

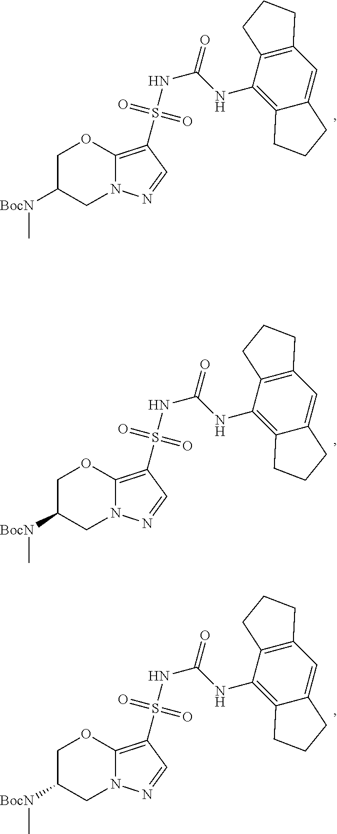 [SMILES: CN(C(=O)OC(C)(C)C)C1COc2c(S(=O)(=O)NC(=O)Nc3c4c(cc5c3CCC5)CCC4)cnn2C1 ; "," ; CN(C(=O)OC(C)(C)C)[C@H]1COc2c(S(=O)(=O)NC(=O)Nc3c4c(cc5c3CCC5)CCC4)cnn2C1 ; "," ; CN(C(=O)OC(C)(C)C)[C@@H]1COc2c(S(=O)(=O)NC(=O)Nc3c4c(cc5c3CCC5)CCC4)cnn2C1 ; ","]

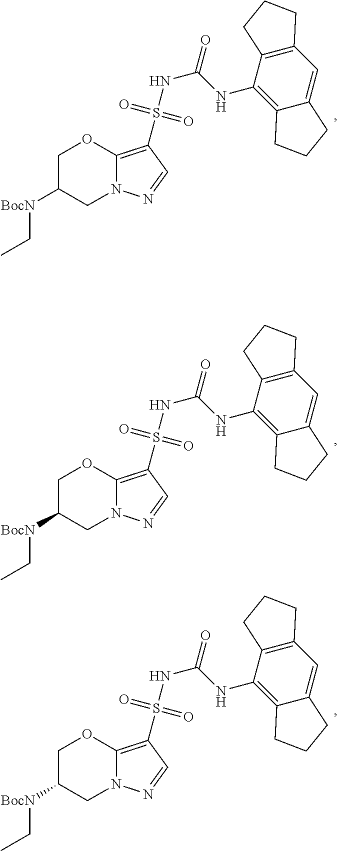 [SMILES: CCN(C(=O)OC(C)(C)C)C1COc2c(S(=O)(=O)NC(=O)Nc3c4c(cc5c3CCC5)CCC4)cnn2C1 ; "," ; CCN(C(=O)OC(C)(C)C)[C@H]1COc2c(S(=O)(=O)NC(=O)Nc3c4c(cc5c3CCC5)CCC4)cnn2C1 ; "," ; CCN(C(=O)OC(C)(C)C)[C@@H]1COc2c(S(=O)(=O)NC(=O)Nc3c4c(cc5c3CCC5)CCC4)cnn2C1 ; ","]

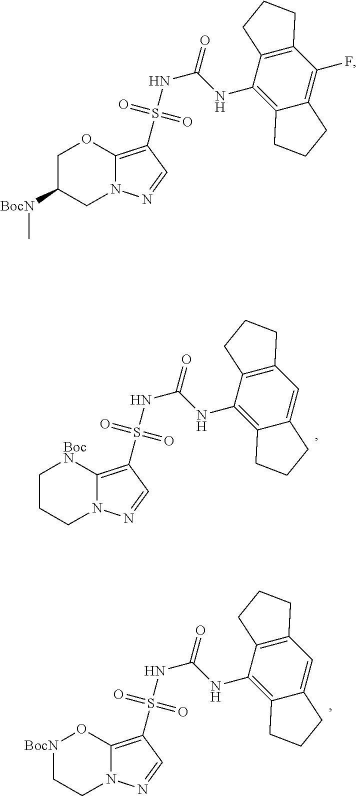 [SMILES: CN(C(=O)OC(C)(C)C)[C@H]1COc2c(S(=O)(=O)NC(=O)Nc3c4c(c(F)c5c3CCC5)CCC4)cnn2C1 ; CC(C)(C)OC(=O)N1CCCn2ncc(S(=O)(=O)NC(=O)Nc3c4c(cc5c3CCC5)CCC4)c21 ; "," ; CC(C)(C)OC(=O)N1CCn2ncc(S(=O)(=O)NC(=O)Nc3c4c(cc5c3CCC5)CCC4)c2O1 ; ","]

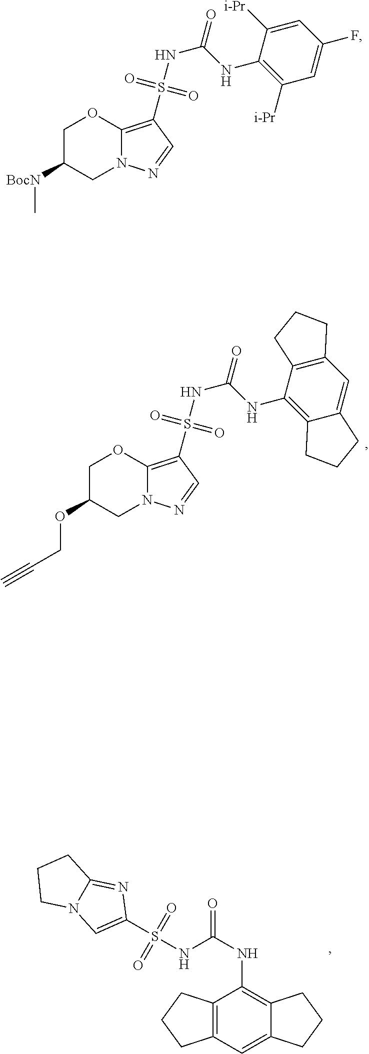 [SMILES: CC(C)c1cc(F)cc(C(C)C)c1NC(=O)NS(=O)(=O)c1cnn2c1OC[C@H](N(C)C(=O)OC(C)(C)C)C2 ; C#CCO[C@H]1COc2c(S(=O)(=O)NC(=O)Nc3c4c(cc5c3CCC5)CCC4)cnn2C1 ; "," ; O=C(Nc1c2c(cc3c1CCC3)CCC2)NS(=O)(=O)c1cn2c(n1)CCC2 ; ","]

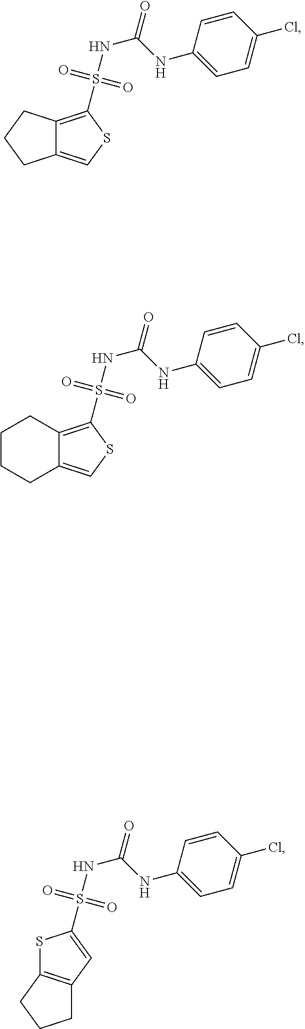 [SMILES: O=C(Nc1ccc(Cl)cc1)NS(=O)(=O)c1scc2c1CCC2 ; O=C(Nc1ccc(Cl)cc1)NS(=O)(=O)c1scc2c1CCCC2 ; O=C(Nc1ccc(Cl)cc1)NS(=O)(=O)c1cc2c(s1)CCC2]

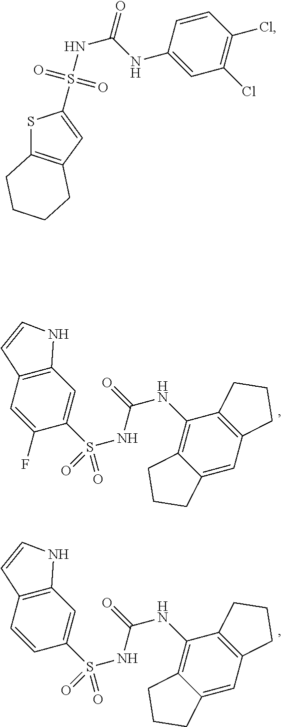 [SMILES: O=C(Nc1ccc(Cl)c(Cl)c1)NS(=O)(=O)c1cc2c(s1)CCCC2 ; O=C(Nc1c2c(cc3c1CCC3)CCC2)NS(=O)(=O)c1cc2[nH]ccc2cc1F ; "," ; O=C(Nc1c2c(cc3c1CCC3)CCC2)NS(=O)(=O)c1ccc2cc[nH]c2c1 ; ","]

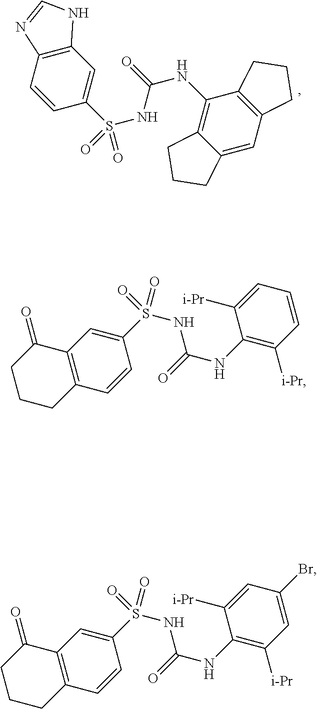 [SMILES: O=C(Nc1c2c(cc3c1CCC3)CCC2)NS(=O)(=O)c1ccc2nc[nH]c2c1 ; "," ; CC(C)c1cccc(C(C)C)c1NC(=O)NS(=O)(=O)c1ccc2c(c1)C(=O)CCC2 ; CC(C)c1cc(Br)cc(C(C)C)c1NC(=O)NS(=O)(=O)c1ccc2c(c1)C(=O)CCC2]

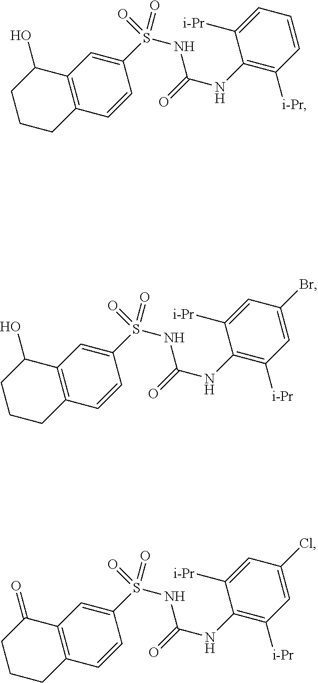 [SMILES: CC(C)c1cccc(C(C)C)c1NC(=O)NS(=O)(=O)c1ccc2c(c1)C(O)CCC2 ; CC(C)c1cc(Br)cc(C(C)C)c1NC(=O)NS(=O)(=O)c1ccc2c(c1)C(O)CCC2 ; CC(C)c1cc(Cl)cc(C(C)C)c1NC(=O)NS(=O)(=O)c1ccc2c(c1)C(=O)CCC2]

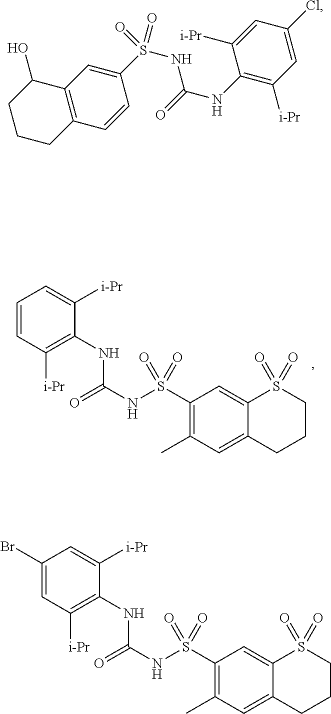 [SMILES: CC(C)c1cc(Cl)cc(C(C)C)c1NC(=O)NS(=O)(=O)c1ccc2c(c1)C(O)CCC2 ; Cc1cc2c(cc1S(=O)(=O)NC(=O)Nc1c(C(C)C)cccc1C(C)C)S(=O)(=O)CCC2 ; "," ; Cc1cc2c(cc1S(=O)(=O)NC(=O)Nc1c(C(C)C)cc(Br)cc1C(C)C)S(=O)(=O)CCC2]

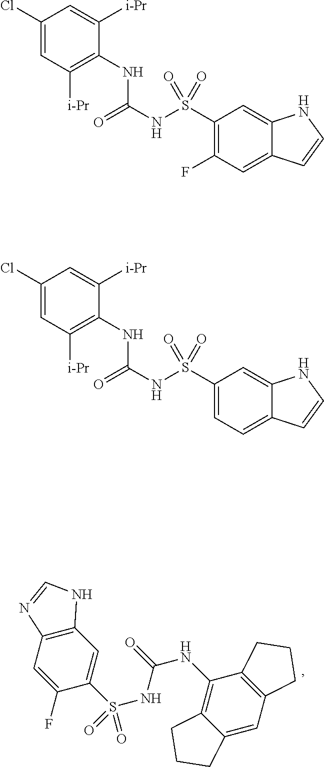 [SMILES: CC(C)c1cc(Cl)cc(C(C)C)c1NC(=O)NS(=O)(=O)c1cc2[nH]ccc2cc1F ; CC(C)c1cc(Cl)cc(C(C)C)c1NC(=O)NS(=O)(=O)c1ccc2cc[nH]c2c1 ; O=C(Nc1c2c(cc3c1CCC3)CCC2)NS(=O)(=O)c1cc2[nH]cnc2cc1F ; ","]

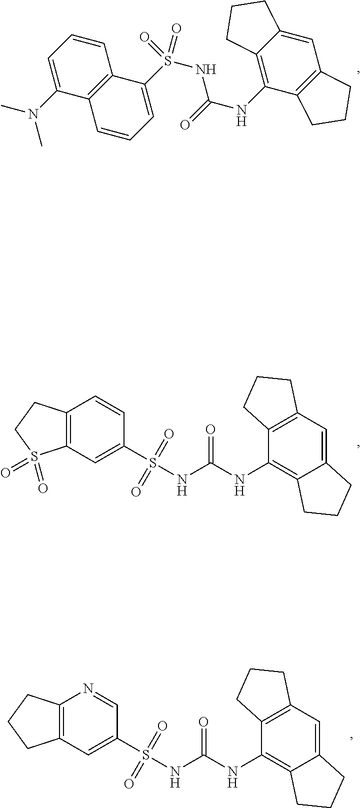 [SMILES: CN(C)c1cccc2c(S(=O)(=O)NC(=O)Nc3c4c(cc5c3CCC5)CCC4)cccc12 ; "," ; O=C(Nc1c2c(cc3c1CCC3)CCC2)NS(=O)(=O)c1ccc2c(c1)S(=O)(=O)CC2 ; "," ; O=C(Nc1c2c(cc3c1CCC3)CCC2)NS(=O)(=O)c1cnc2c(c1)CCC2 ; ","]

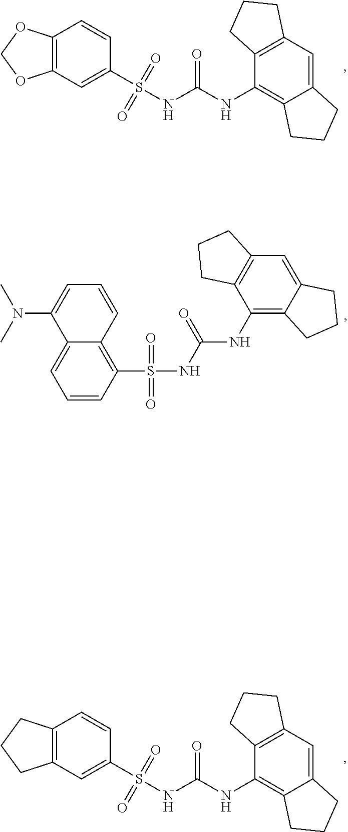 [SMILES: O=C(Nc1c2c(cc3c1CCC3)CCC2)NS(=O)(=O)c1ccc2c(c1)OCO2 ; "," ; CN(C)c1cccc2c(S(=O)(=O)NC(=O)Nc3c4c(cc5c3CCC5)CCC4)cccc12 ; "," ; O=C(Nc1c2c(cc3c1CCC3)CCC2)NS(=O)(=O)c1ccc2c(c1)CCC2 ; ","]

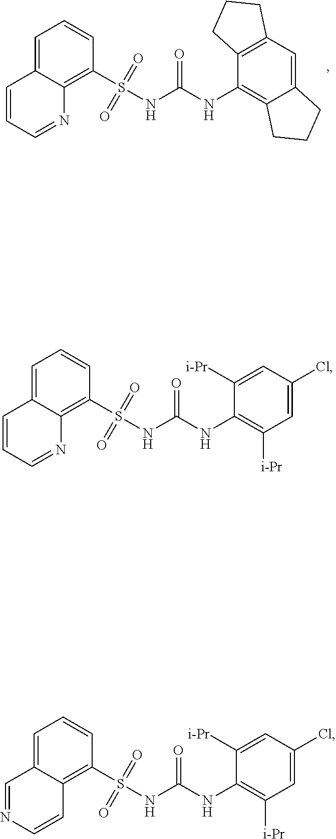 [SMILES: O=C(Nc1c2c(cc3c1CCC3)CCC2)NS(=O)(=O)c1cccc2cccnc12 ; "," ; CC(C)c1cc(Cl)cc(C(C)C)c1NC(=O)NS(=O)(=O)c1cccc2cccnc12 ; CC(C)c1cc(Cl)cc(C(C)C)c1NC(=O)NS(=O)(=O)c1cccc2cnccc12]

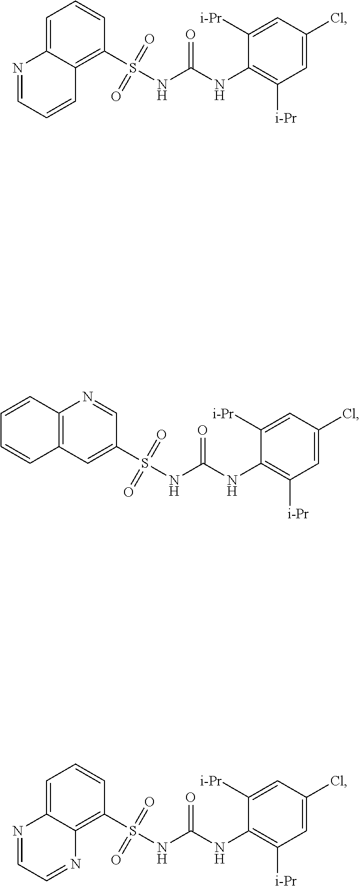 [SMILES: CC(C)c1cc(Cl)cc(C(C)C)c1NC(=O)NS(=O)(=O)c1cccc2ncccc12 ; CC(C)c1cc(Cl)cc(C(C)C)c1NC(=O)NS(=O)(=O)c1cnc2ccccc2c1 ; CC(C)c1cc(Cl)cc(C(C)C)c1NC(=O)NS(=O)(=O)c1cccc2nccnc12]

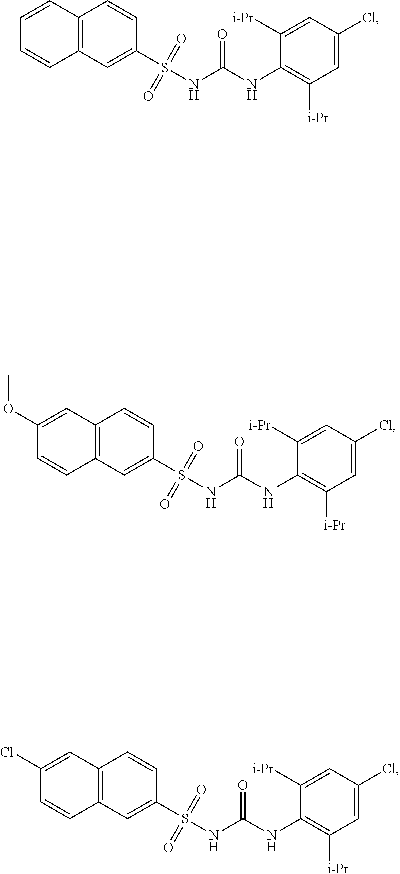 [SMILES: CC(C)c1cc(Cl)cc(C(C)C)c1NC(=O)NS(=O)(=O)c1ccc2ccccc2c1 ; COc1ccc2cc(S(=O)(=O)NC(=O)Nc3c(C(C)C)cc(Cl)cc3C(C)C)ccc2c1 ; CC(C)c1cc(Cl)cc(C(C)C)c1NC(=O)NS(=O)(=O)c1ccc2cc(Cl)ccc2c1]

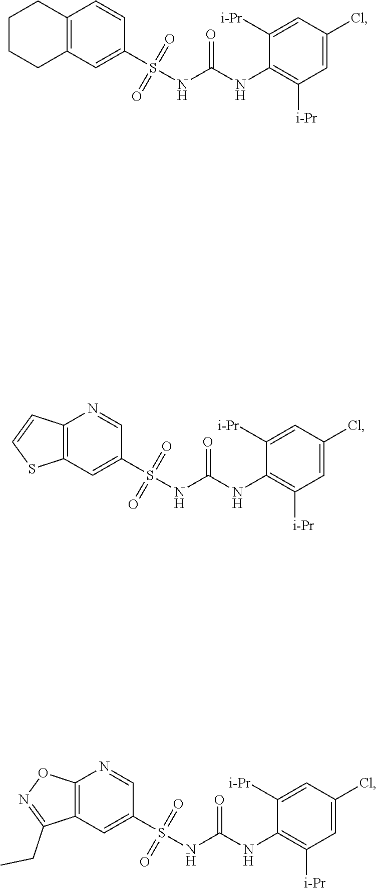 [SMILES: CC(C)c1cc(Cl)cc(C(C)C)c1NC(=O)NS(=O)(=O)c1ccc2c(c1)CCCC2 ; CC(C)c1cc(Cl)cc(C(C)C)c1NC(=O)NS(=O)(=O)c1cnc2ccsc2c1 ; CCc1noc2ncc(S(=O)(=O)NC(=O)Nc3c(C(C)C)cc(Cl)cc3C(C)C)cc12]

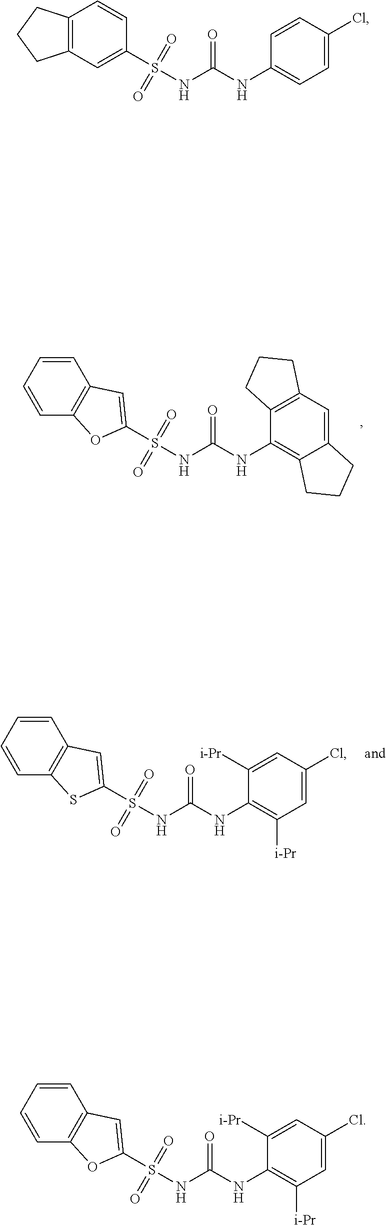 [SMILES: O=C(Nc1ccc(Cl)cc1)NS(=O)(=O)c1ccc2c(c1)CCC2 ; O=C(Nc1c2c(cc3c1CCC3)CCC2)NS(=O)(=O)c1cc2ccccc2o1 ; "," ; CC(C)c1cc(Cl)cc(C(C)C)c1NC(=O)NS(=O)(=O)c1cc2ccccc2s1 ; "and" ; CC(C)c1cc(Cl)cc(C(C)C)c1NC(=O)NS(=O)(=O)c1cc2ccccc2o1]

In some embodiments, the compound of Formula AA is not a compound selected from the group consisting of:

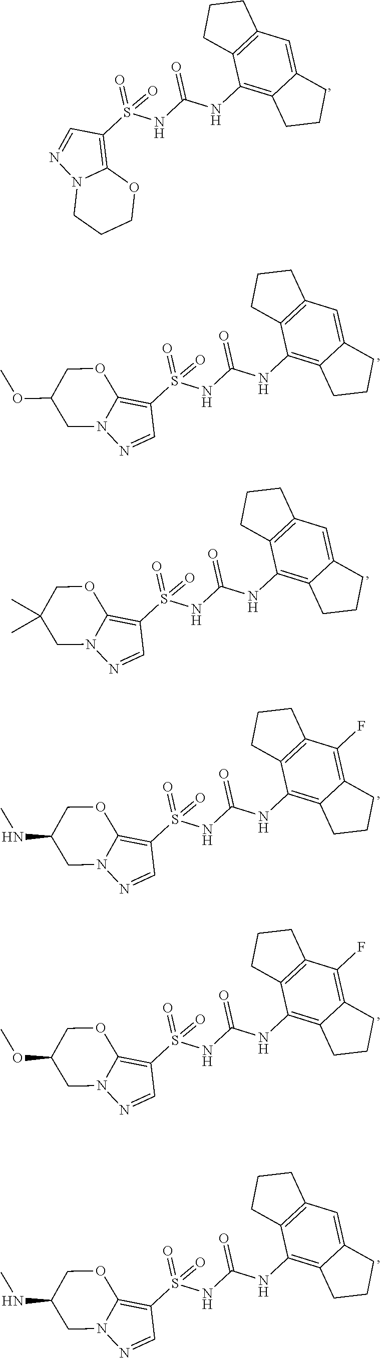 [SMILES: O=C(Nc1c2c(cc3c1CCC3)CCC2)NS(=O)(=O)c1cnn2c1OCCC2 ; COC1COc2c(S(=O)(=O)NC(=O)Nc3c4c(cc5c3CCC5)CCC4)cnn2C1 ; CC1(C)COc2c(S(=O)(=O)NC(=O)Nc3c4c(cc5c3CCC5)CCC4)cnn2C1 ; CN[C@H]1COc2c(S(=O)(=O)NC(=O)Nc3c4c(c(F)c5c3CCC5)CCC4)cnn2C1 ; CO[C@H]1COc2c(S(=O)(=O)NC(=O)Nc3c4c(c(F)c5c3CCC5)CCC4)cnn2C1 ; CN[C@H]1COc2c(S(=O)(=O)NC(=O)Nc3c4c(cc5c3CCC5)CCC4)cnn2C1]

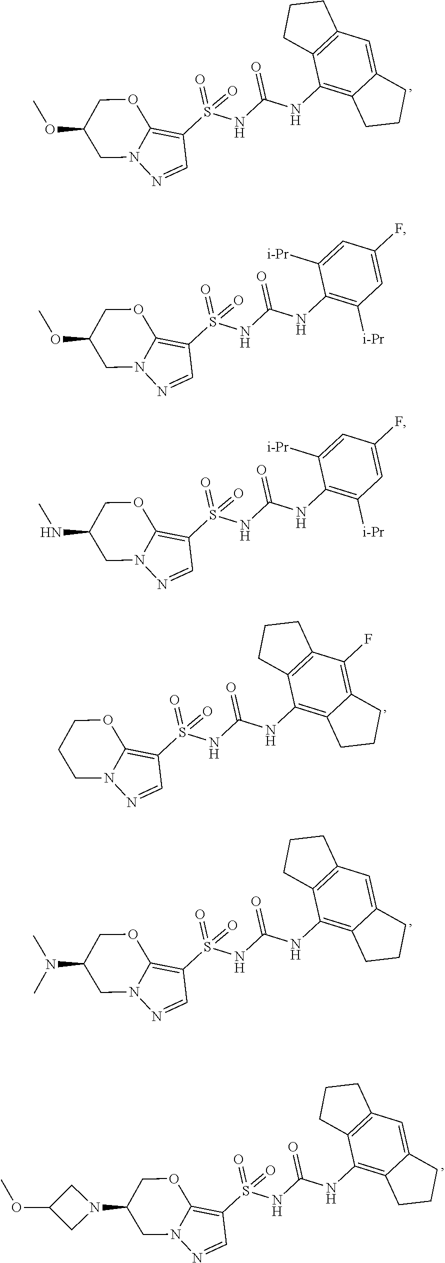 [SMILES: CO[C@H]1COc2c(S(=O)(=O)NC(=O)Nc3c4c(cc5c3CCC5)CCC4)cnn2C1 ; CO[C@H]1COc2c(S(=O)(=O)NC(=O)Nc3c(C(C)C)cc(F)cc3C(C)C)cnn2C1 ; CN[C@H]1COc2c(S(=O)(=O)NC(=O)Nc3c(C(C)C)cc(F)cc3C(C)C)cnn2C1 ; O=C(Nc1c2c(c(F)c3c1CCC3)CCC2)NS(=O)(=O)c1cnn2c1OCCC2 ; CN(C)[C@H]1COc2c(S(=O)(=O)NC(=O)Nc3c4c(cc5c3CCC5)CCC4)cnn2C1 ; COC1CN([C@H]2COc3c(S(=O)(=O)NC(=O)Nc4c5c(cc6c4CCC6)CCC5)cnn3C2)C1]

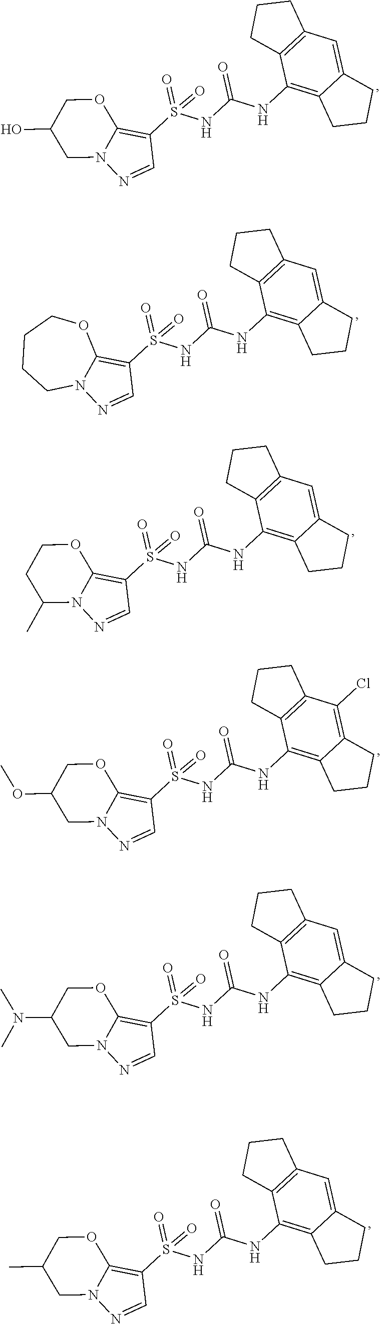 [SMILES: O=C(Nc1c2c(cc3c1CCC3)CCC2)NS(=O)(=O)c1cnn2c1OCC(O)C2 ; O=C(Nc1c2c(cc3c1CCC3)CCC2)NS(=O)(=O)c1cnn2c1OCCCC2 ; CC1CCOc2c(S(=O)(=O)NC(=O)Nc3c4c(cc5c3CCC5)CCC4)cnn21 ; COC1COc2c(S(=O)(=O)NC(=O)Nc3c4c(c(Cl)c5c3CCC5)CCC4)cnn2C1 ; CN(C)C1COc2c(S(=O)(=O)NC(=O)Nc3c4c(cc5c3CCC5)CCC4)cnn2C1 ; CC1COc2c(S(=O)(=O)NC(=O)Nc3c4c(cc5c3CCC5)CCC4)cnn2C1]

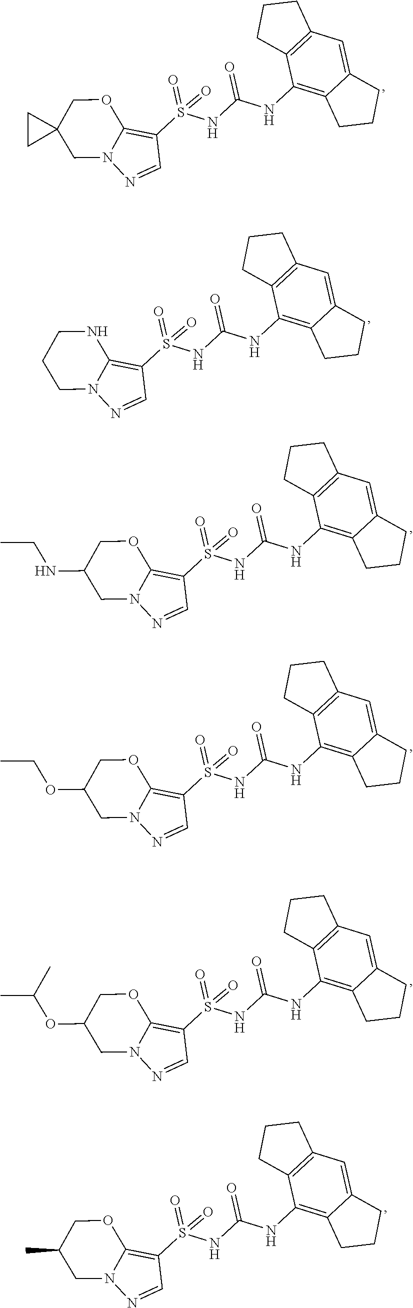 [SMILES: O=C(Nc1c2c(cc3c1CCC3)CCC2)NS(=O)(=O)c1cnn2c1OCC1(CC1)C2 ; O=C(Nc1c2c(cc3c1CCC3)CCC2)NS(=O)(=O)c1cnn2c1NCCC2 ; CCNC1COc2c(S(=O)(=O)NC(=O)Nc3c4c(cc5c3CCC5)CCC4)cnn2C1 ; CCOC1COc2c(S(=O)(=O)NC(=O)Nc3c4c(cc5c3CCC5)CCC4)cnn2C1 ; CC(C)OC1COc2c(S(=O)(=O)NC(=O)Nc3c4c(cc5c3CCC5)CCC4)cnn2C1 ; C[C@H]1COc2c(S(=O)(=O)NC(=O)Nc3c4c(cc5c3CCC5)CCC4)cnn2C1]

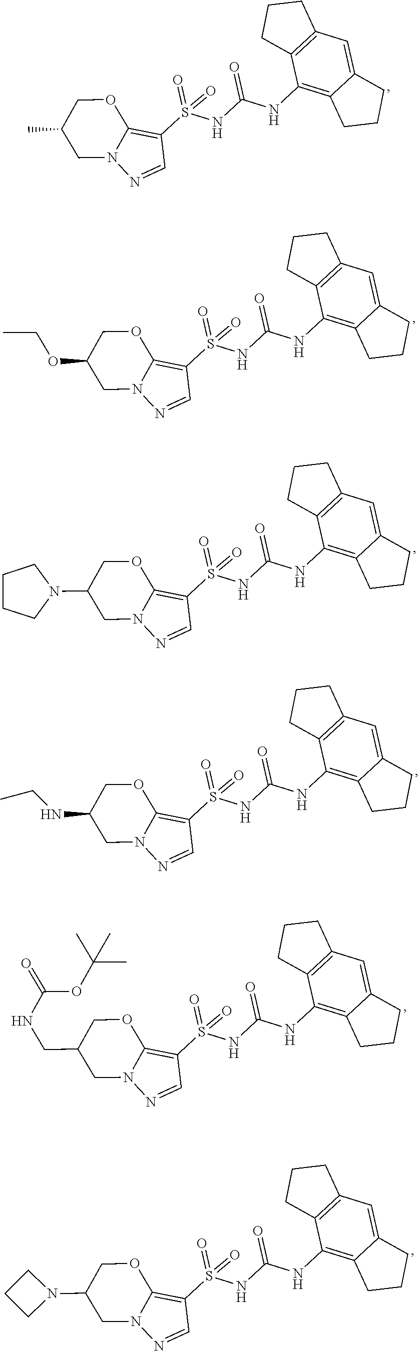 [SMILES: C[C@@H]1COc2c(S(=O)(=O)NC(=O)Nc3c4c(cc5c3CCC5)CCC4)cnn2C1 ; CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCCC1)C2 ; CCN[C@H]1COc2c(S(=O)(=O)NC(=O)Nc3c4c(cc5c3CCC5)CCC4)cnn2C1 ; CC(C)(C)OC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCC1)C2]

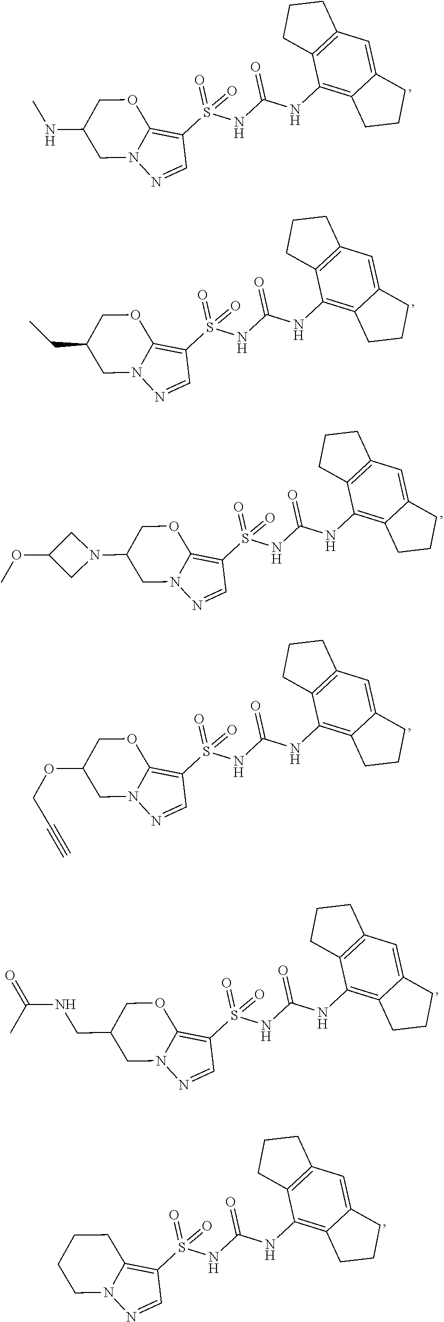 [SMILES: CNC1COc2c(S(=O)(=O)NC(=O)Nc3c4c(cc5c3CCC5)CCC4)cnn2C1 ; CC[C@H]1COc2c(S(=O)(=O)NC(=O)Nc3c4c(cc5c3CCC5)CCC4)cnn2C1 ; COC1CN(C2COc3c(S(=O)(=O)NC(=O)Nc4c5c(cc6c4CCC6)CCC5)cnn3C2)C1 ; C#CCOC1COc2c(S(=O)(=O)NC(=O)Nc3c4c(cc5c3CCC5)CCC4)cnn2C1 ; CC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1CCCC2]

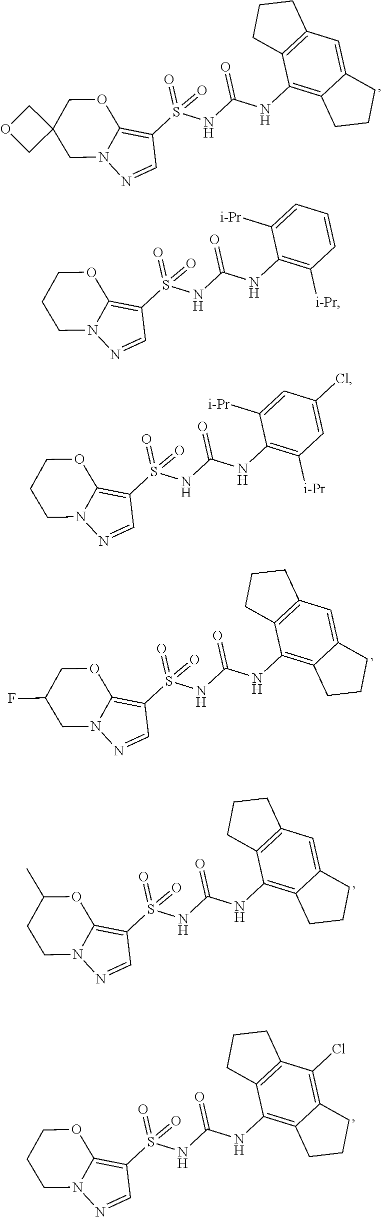 [SMILES: O=C(Nc1c2c(cc3c1CCC3)CCC2)NS(=O)(=O)c1cnn2c1OCC1(COC1)C2 ; CC(C)c1cccc(C(C)C)c1NC(=O)NS(=O)(=O)c1cnn2c1OCCC2 ; CC(C)c1cc(Cl)cc(C(C)C)c1NC(=O)NS(=O)(=O)c1cnn2c1OCCC2 ; O=C(Nc1c2c(cc3c1CCC3)CCC2)NS(=O)(=O)c1cnn2c1OCC(F)C2 ; CC1CCn2ncc(S(=O)(=O)NC(=O)Nc3c4c(cc5c3CCC5)CCC4)c2O1 ; O=C(Nc1c2c(c(Cl)c3c1CCC3)CCC2)NS(=O)(=O)c1cnn2c1OCCC2]

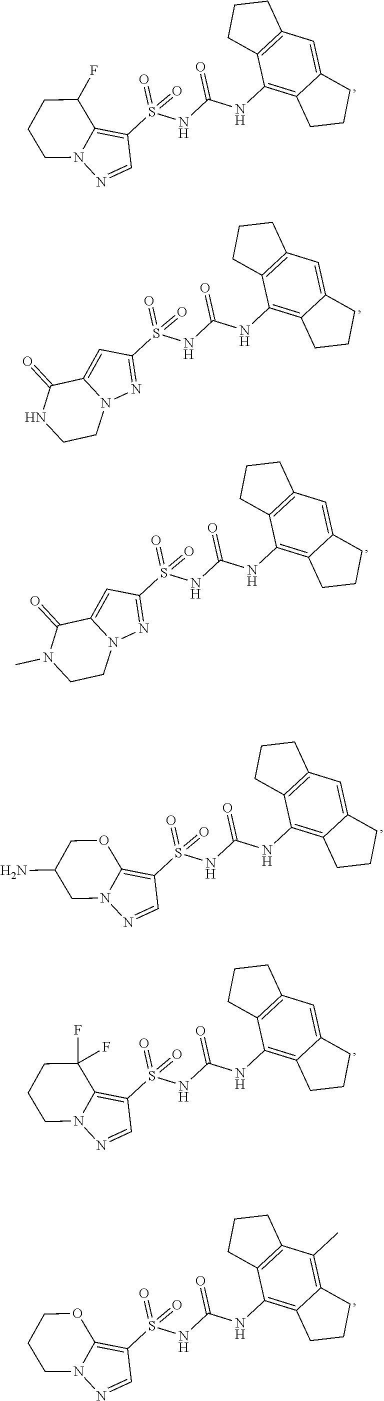 [SMILES: O=C(Nc1c2c(cc3c1CCC3)CCC2)NS(=O)(=O)c1cnn2c1C(F)CCC2 ; O=C(Nc1c2c(cc3c1CCC3)CCC2)NS(=O)(=O)c1cc2n(n1)CCNC2=O ; CN1CCn2nc(S(=O)(=O)NC(=O)Nc3c4c(cc5c3CCC5)CCC4)cc2C1=O ; NC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1C(F)(F)CCC2 ; Cc1c2c(c(NC(=O)NS(=O)(=O)c3cnn4c3OCCC4)c3c1CCC3)CCC2]

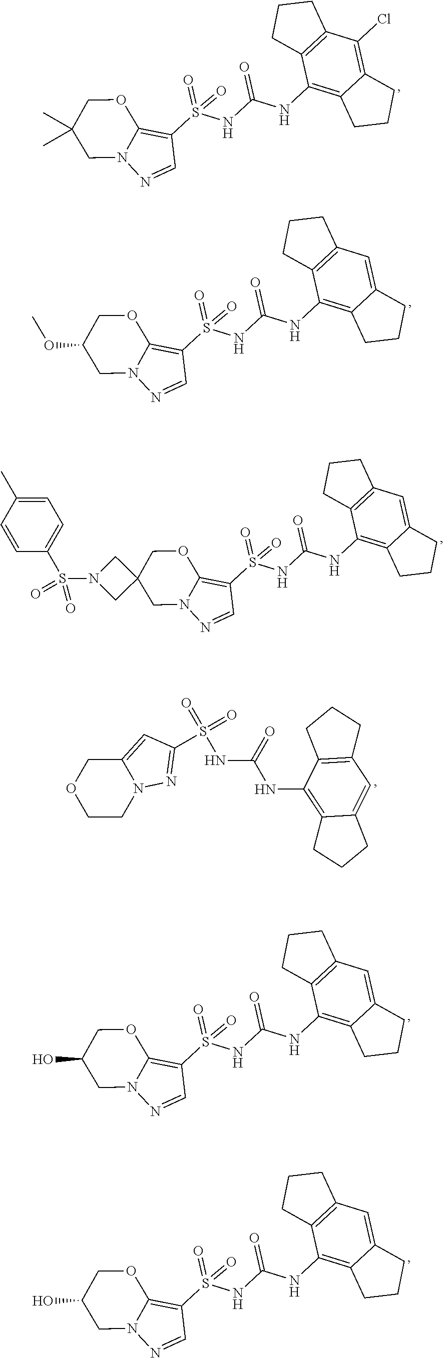 [SMILES: CC1(C)COc2c(S(=O)(=O)NC(=O)Nc3c4c(c(Cl)c5c3CCC5)CCC4)cnn2C1 ; CO[C@@H]1COc2c(S(=O)(=O)NC(=O)Nc3c4c(cc5c3CCC5)CCC4)cnn2C1 ; Cc1ccc(S(=O)(=O)N2CC3(COc4c(S(=O)(=O)NC(=O)Nc5c6c(cc7c5CCC7)CCC6)cnn4C3)C2)cc1 ; O=C(Nc1c2c(cc3c1CCC3)CCC2)NS(=O)(=O)c1cc2n(n1)CCOC2 ; O=C(Nc1c2c(cc3c1CCC3)CCC2)NS(=O)(=O)c1cnn2c1OC[C@H](O)C2 ; O=C(Nc1c2c(cc3c1CCC3)CCC2)NS(=O)(=O)c1cnn2c1OC[C@@H](O)C2]

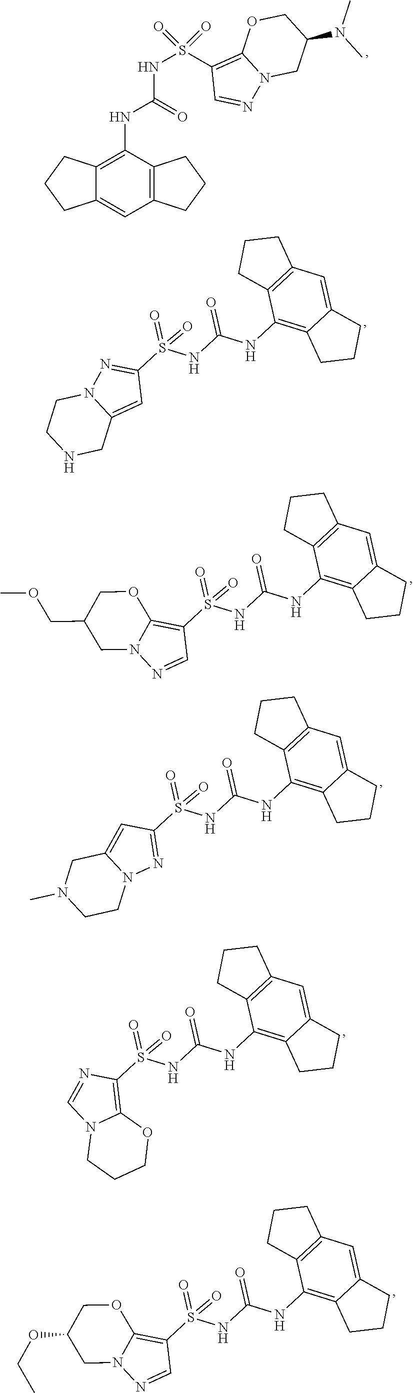

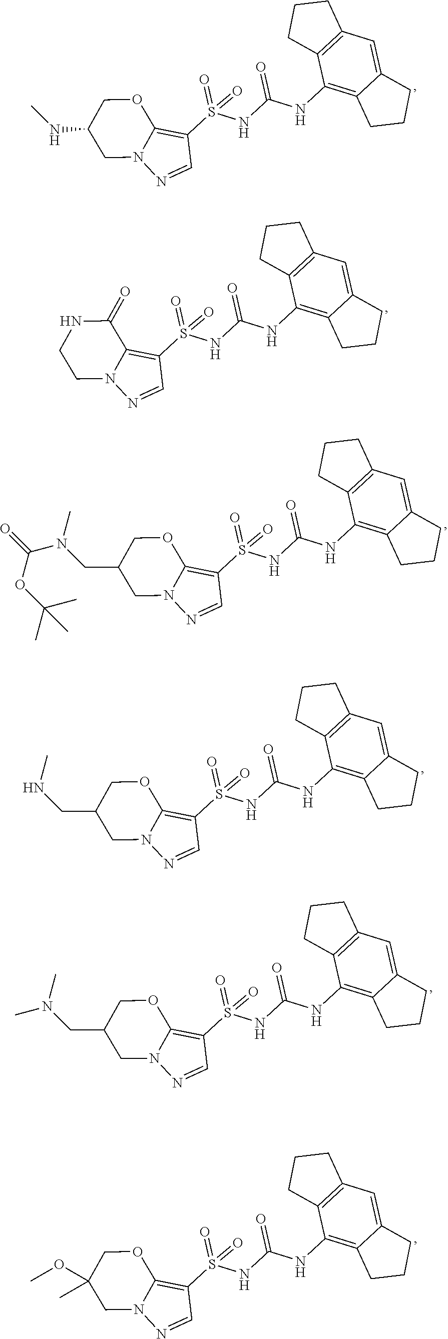 [SMILES: CN[C@@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1C(=O)NCC2 ; CN(CC1COc2c(S(=O)(=O)NC(=O)Nc3c4c(cc5c3CCC5)CCC4)cnn2C1)C(=O)OC(C)(C)C ; CNCC1COc2c(S(=O)(=O)NC(=O)Nc3c4c(cc5c3CCC5)CCC4)cnn2C1 ; CN(C)CC1COc2c(S(=O)(=O)NC(=O)Nc3c4c(cc5c3CCC5)CCC4)cnn2C1 ; COC1(C)COc2c(S(=O)(=O)NC(=O)Nc3c4c(cc5c3CCC5)CCC4)cnn2C1]

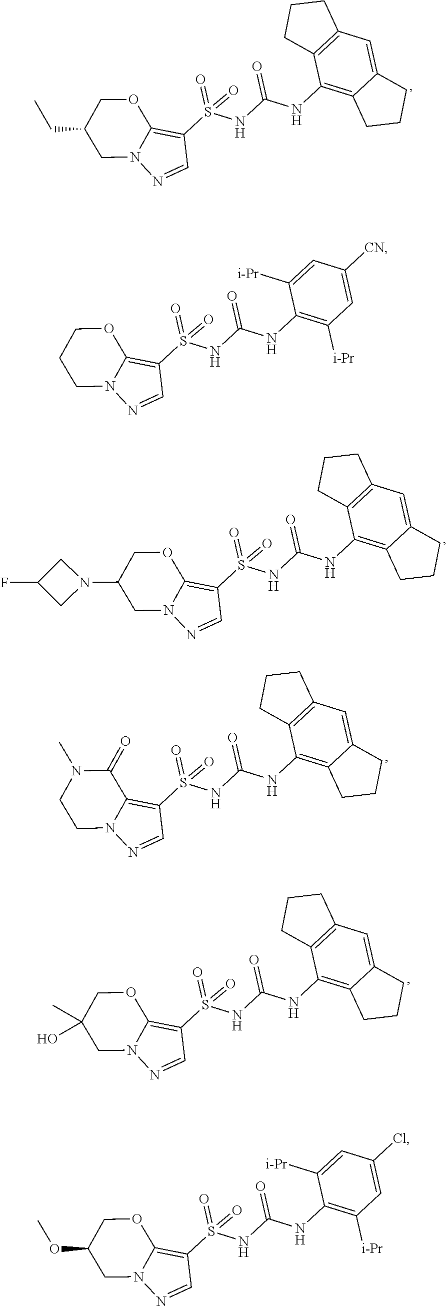 [SMILES: CC[C@@H]1COc2c(S(=O)(=O)NC(=O)Nc3c4c(cc5c3CCC5)CCC4)cnn2C1 ; CC(C)c1cc(C#N)cc(C(C)C)c1NC(=O)NS(=O)(=O)c1cnn2c1OCCC2 ; O=C(Nc1c2c(cc3c1CCC3)CCC2)NS(=O)(=O)c1cnn2c1OCC(N1CC(F)C1)C2 ; CN1CCn2ncc(S(=O)(=O)NC(=O)Nc3c4c(cc5c3CCC5)CCC4)c2C1=O ; CC1(O)COc2c(S(=O)(=O)NC(=O)Nc3c4c(cc5c3CCC5)CCC4)cnn2C1 ; CO[C@H]1COc2c(S(=O)(=O)NC(=O)Nc3c(C(C)C)cc(Cl)cc3C(C)C)cnn2C1]

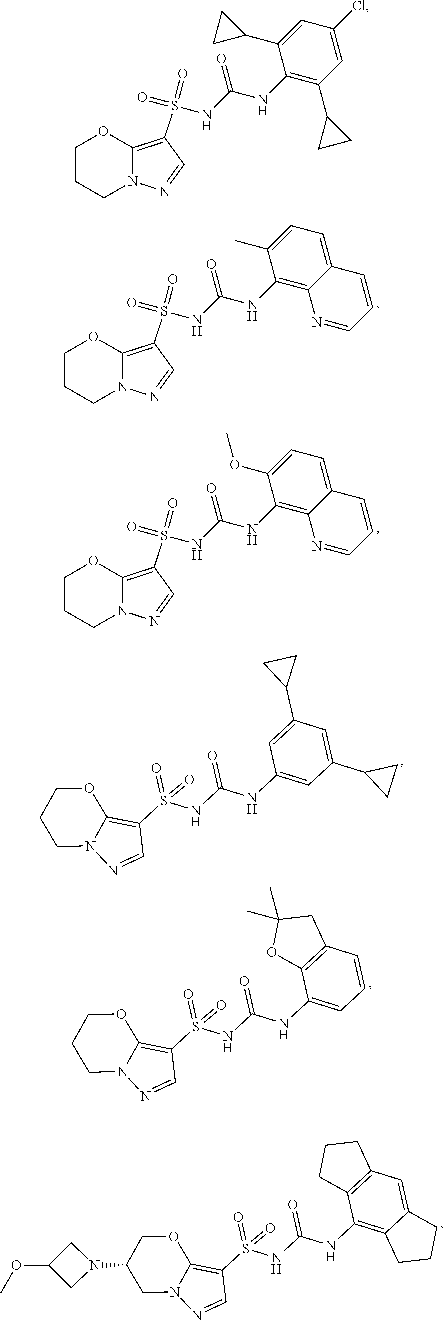 [SMILES: O=C(Nc1c(C2CC2)cc(Cl)cc1C1CC1)NS(=O)(=O)c1cnn2c1OCCC2 ; Cc1ccc2cccnc2c1NC(=O)NS(=O)(=O)c1cnn2c1OCCC2 ; COc1ccc2cccnc2c1NC(=O)NS(=O)(=O)c1cnn2c1OCCC2 ; O=C(Nc1cc(C2CC2)cc(C2CC2)c1)NS(=O)(=O)c1cnn2c1OCCC2 ; CC1(C)Cc2cccc(NC(=O)NS(=O)(=O)c3cnn4c3OCCC4)c2O1 ; COC1CN([C@@H]2COc3c(S(=O)(=O)NC(=O)Nc4c5c(cc6c4CCC6)CCC5)cnn3C2)C1]

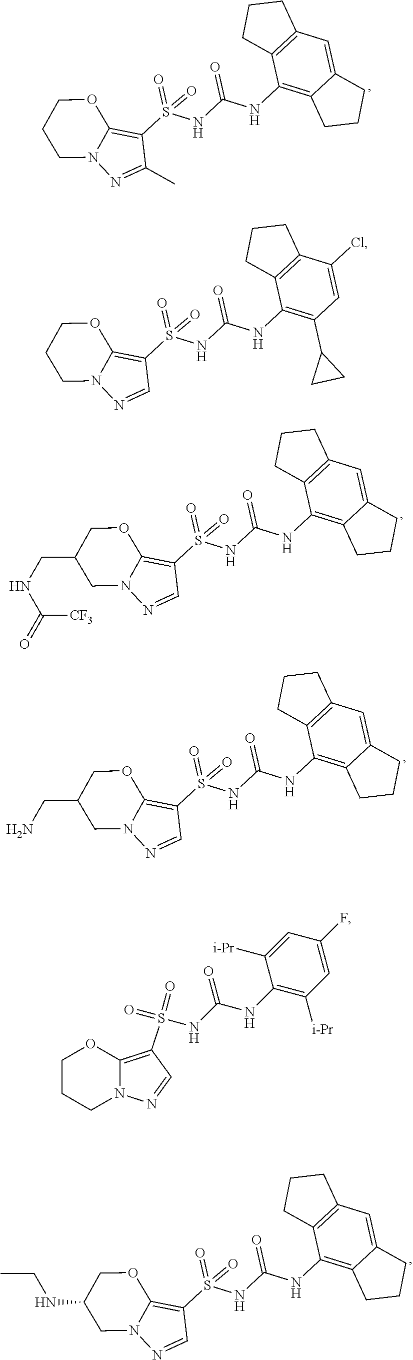

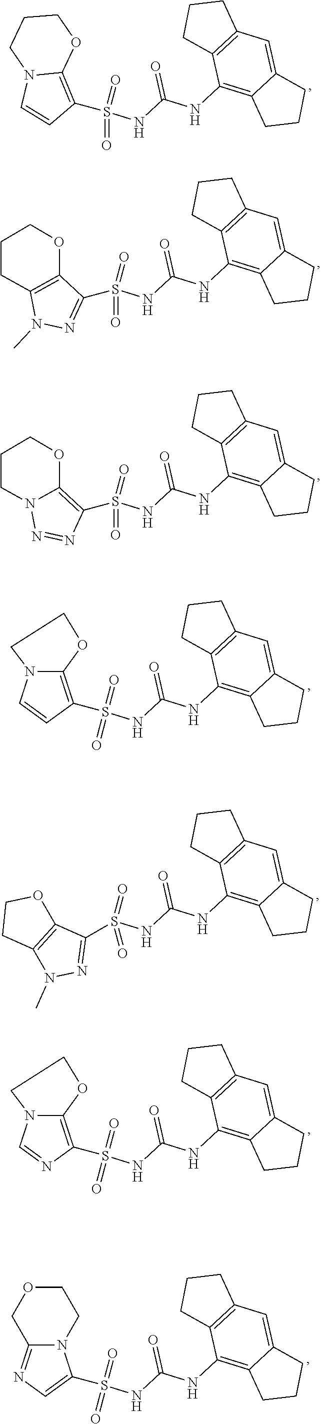

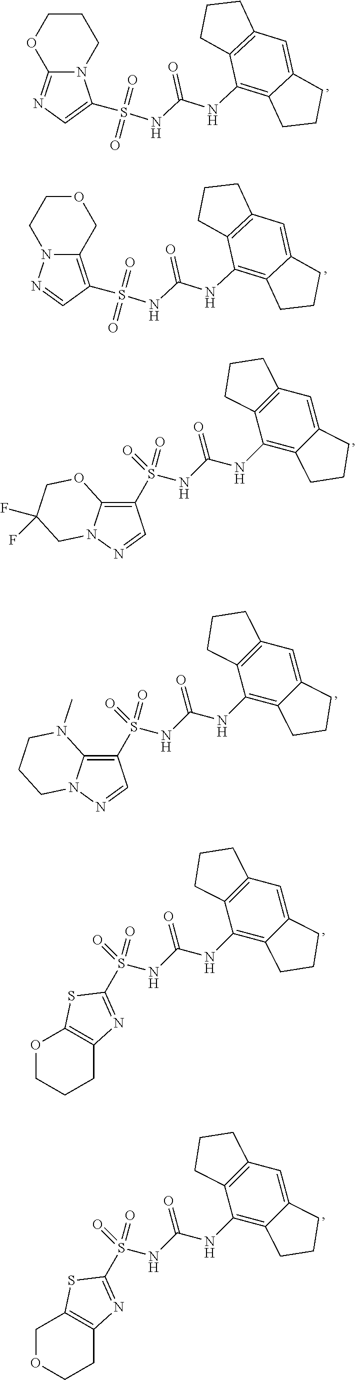 [SMILES: O=C(Nc1c2c(cc3c1CCC3)CCC2)NS(=O)(=O)c1cnc2n1CCCO2 ; O=C(Nc1c2c(cc3c1CCC3)CCC2)NS(=O)(=O)c1cnn2c1COCC2 ; O=C(Nc1c2c(cc3c1CCC3)CCC2)NS(=O)(=O)c1cnn2c1OCC(F)(F)C2 ; CN1CCCn2ncc(S(=O)(=O)NC(=O)Nc3c4c(cc5c3CCC5)CCC4)c21 ; O=C(Nc1c2c(cc3c1CCC3)CCC2)NS(=O)(=O)c1nc2c(s1)OCCC2 ; O=C(Nc1c2c(cc3c1CCC3)CCC2)NS(=O)(=O)c1nc2c(s1)COCC2]

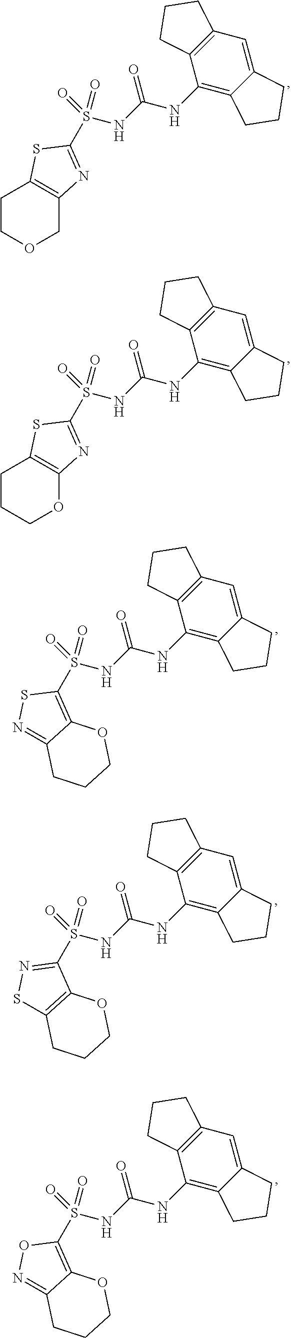 [SMILES: O=C(Nc1c2c(cc3c1CCC3)CCC2)NS(=O)(=O)c1nc2c(s1)CCOC2 ; O=C(Nc1c2c(cc3c1CCC3)CCC2)NS(=O)(=O)c1nc2c(s1)CCCO2 ; O=C(Nc1c2c(cc3c1CCC3)CCC2)NS(=O)(=O)c1snc2c1OCCC2 ; O=C(Nc1c2c(cc3c1CCC3)CCC2)NS(=O)(=O)c1nsc2c1OCCC2 ; O=C(Nc1c2c(cc3c1CCC3)CCC2)NS(=O)(=O)c1onc2c1OCCC2]

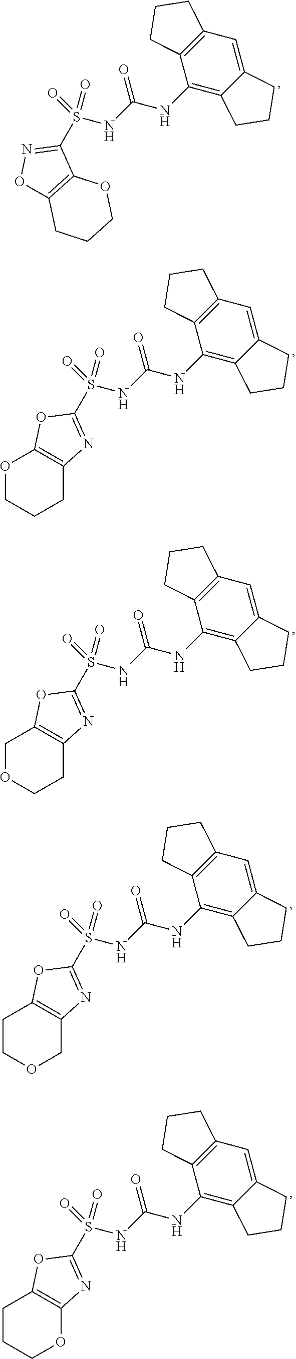 [SMILES: O=C(Nc1c2c(cc3c1CCC3)CCC2)NS(=O)(=O)c1noc2c1OCCC2 ; O=C(Nc1c2c(cc3c1CCC3)CCC2)NS(=O)(=O)c1nc2c(o1)OCCC2 ; O=C(Nc1c2c(cc3c1CCC3)CCC2)NS(=O)(=O)c1nc2c(o1)COCC2 ; O=C(Nc1c2c(cc3c1CCC3)CCC2)NS(=O)(=O)c1nc2c(o1)CCOC2 ; O=C(Nc1c2c(cc3c1CCC3)CCC2)NS(=O)(=O)c1nc2c(o1)CCCO2]

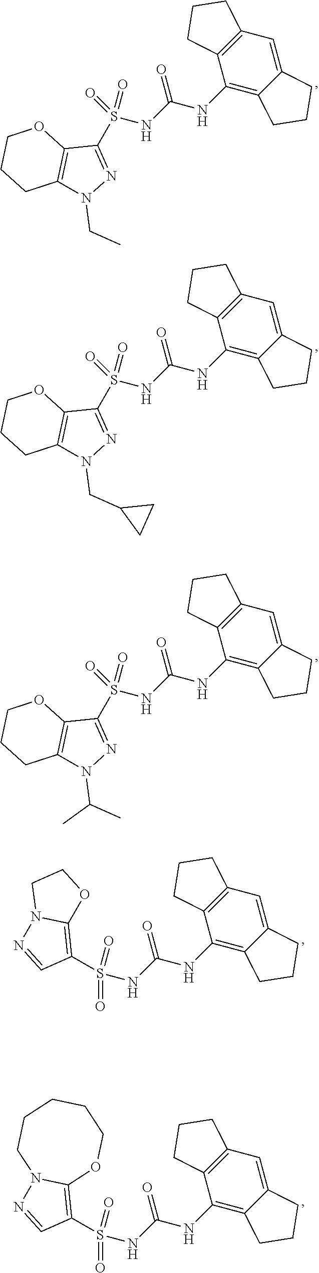

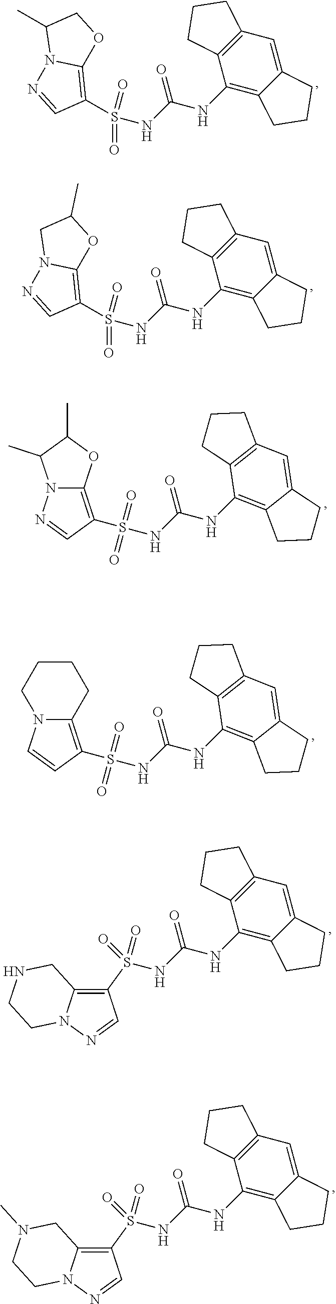 [SMILES: CC1COc2c(S(=O)(=O)NC(=O)Nc3c4c(cc5c3CCC5)CCC4)cnn21 ; CC1Cn2ncc(S(=O)(=O)NC(=O)Nc3c4c(cc5c3CCC5)CCC4)c2O1 ; CC1Oc2c(S(=O)(=O)NC(=O)Nc3c4c(cc5c3CCC5)CCC4)cnn2C1C ; O=C(Nc1c2c(cc3c1CCC3)CCC2)NS(=O)(=O)c1ccn2c1CCCC2 ; O=C(Nc1c2c(cc3c1CCC3)CCC2)NS(=O)(=O)c1cnn2c1CNCC2 ; CN1CCn2ncc(S(=O)(=O)NC(=O)Nc3c4c(cc5c3CCC5)CCC4)c2C1]

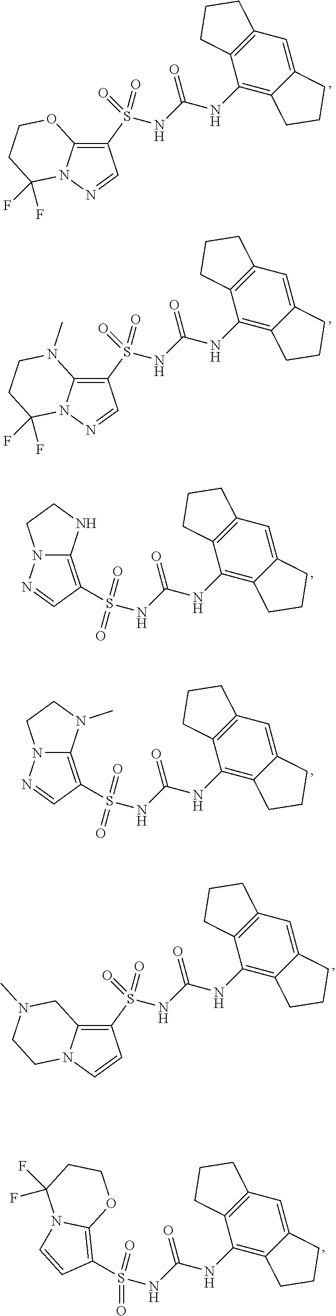 [SMILES: O=C(Nc1c2c(cc3c1CCC3)CCC2)NS(=O)(=O)c1cnn2c1OCCC2(F)F ; CN1CCC(F)(F)n2ncc(S(=O)(=O)NC(=O)Nc3c4c(cc5c3CCC5)CCC4)c21 ; O=C(Nc1c2c(cc3c1CCC3)CCC2)NS(=O)(=O)c1cnn2c1NCC2 ; CN1CCn2ncc(S(=O)(=O)NC(=O)Nc3c4c(cc5c3CCC5)CCC4)c21 ; CN1CCn2ccc(S(=O)(=O)NC(=O)Nc3c4c(cc5c3CCC5)CCC4)c2C1 ; O=C(Nc1c2c(cc3c1CCC3)CCC2)NS(=O)(=O)c1ccn2c1OCCC2(F)F]

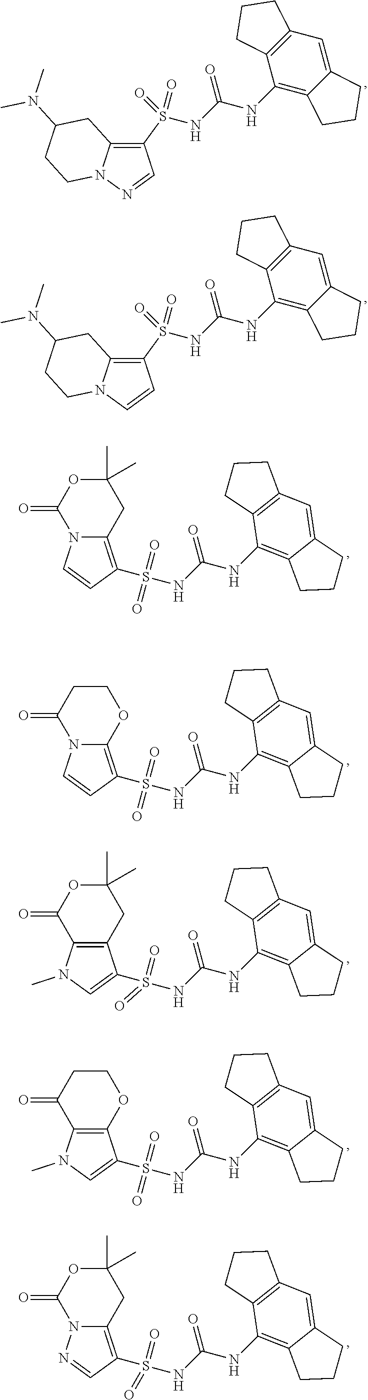

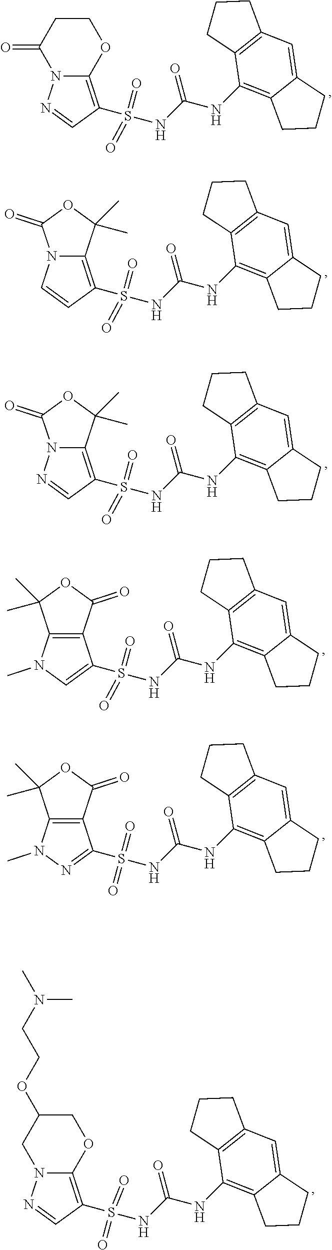 [SMILES: O=C(Nc1c2c(cc3c1CCC3)CCC2)NS(=O)(=O)c1cnn2c1OCCC2=O ; CC1(C)OC(=O)n2ccc(S(=O)(=O)NC(=O)Nc3c4c(cc5c3CCC5)CCC4)c21 ; CC1(C)OC(=O)n2ncc(S(=O)(=O)NC(=O)Nc3c4c(cc5c3CCC5)CCC4)c21 ; Cn1cc(S(=O)(=O)NC(=O)Nc2c3c(cc4c2CCC4)CCC3)c2c1C(C)(C)OC2=O ; Cn1nc(S(=O)(=O)NC(=O)Nc2c3c(cc4c2CCC4)CCC3)c2c1C(C)(C)OC2=O ; CN(C)CCOC1COc2c(S(=O)(=O)NC(=O)Nc3c4c(cc5c3CCC5)CCC4)cnn2C1]

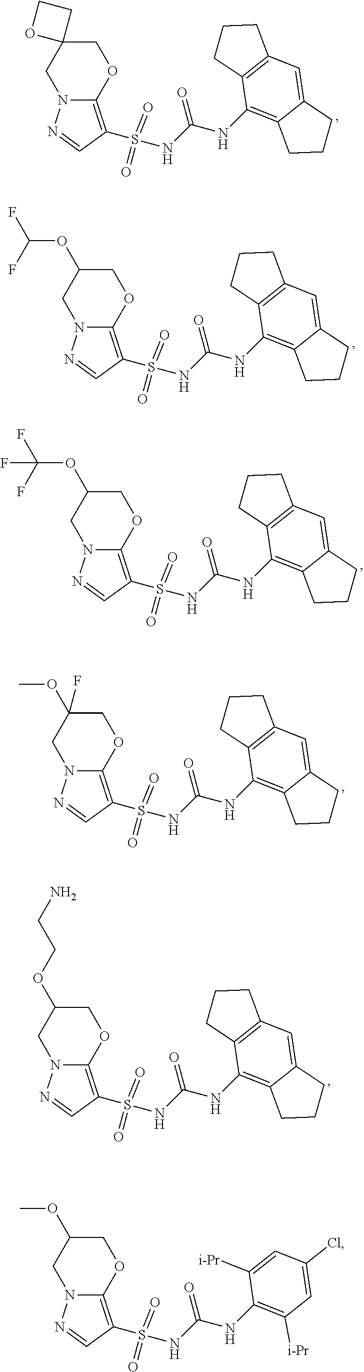 [SMILES: O=C(Nc1c2c(cc3c1CCC3)CCC2)NS(=O)(=O)c1cnn2c1OCC1(CCO1)C2 ; O=C(Nc1c2c(cc3c1CCC3)CCC2)NS(=O)(=O)c1cnn2c1OCC(OC(F)F)C2 ; O=C(Nc1c2c(cc3c1CCC3)CCC2)NS(=O)(=O)c1cnn2c1OCC(OC(F)(F)F)C2 ; COC1(F)COc2c(S(=O)(=O)NC(=O)Nc3c4c(cc5c3CCC5)CCC4)cnn2C1 ; NCCOC1COc2c(S(=O)(=O)NC(=O)Nc3c4c(cc5c3CCC5)CCC4)cnn2C1 ; COC1COc2c(S(=O)(=O)NC(=O)Nc3c(C(C)C)cc(Cl)cc3C(C)C)cnn2C1]

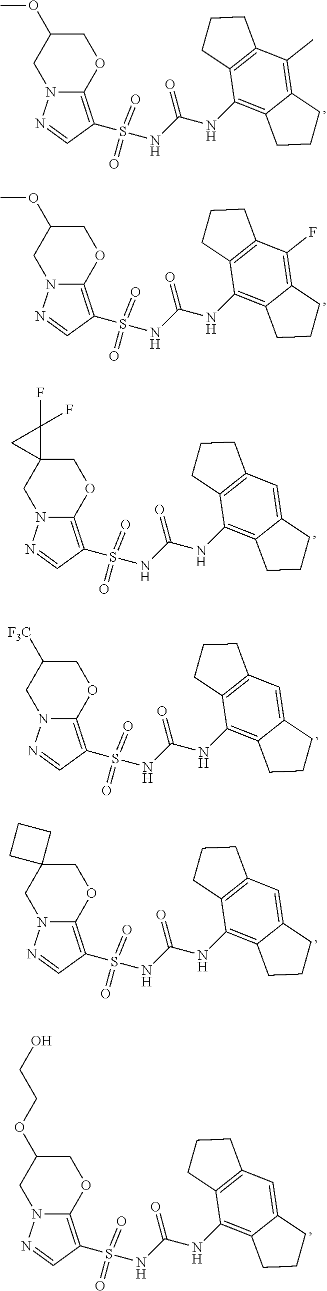 [SMILES: COC1COc2c(S(=O)(=O)NC(=O)Nc3c4c(c(C)c5c3CCC5)CCC4)cnn2C1 ; COC1COc2c(S(=O)(=O)NC(=O)Nc3c4c(c(F)c5c3CCC5)CCC4)cnn2C1 ; O=C(Nc1c2c(cc3c1CCC3)CCC2)NS(=O)(=O)c1cnn2c1OCC1(C2)CC1(F)F ; O=C(Nc1c2c(cc3c1CCC3)CCC2)NS(=O)(=O)c1cnn2c1OCC(C(F)(F)F)C2 ; O=C(Nc1c2c(cc3c1CCC3)CCC2)NS(=O)(=O)c1cnn2c1OCC1(CCC1)C2 ; O=C(Nc1c2c(cc3c1CCC3)CCC2)NS(=O)(=O)c1cnn2c1OCC(OCCO)C2]

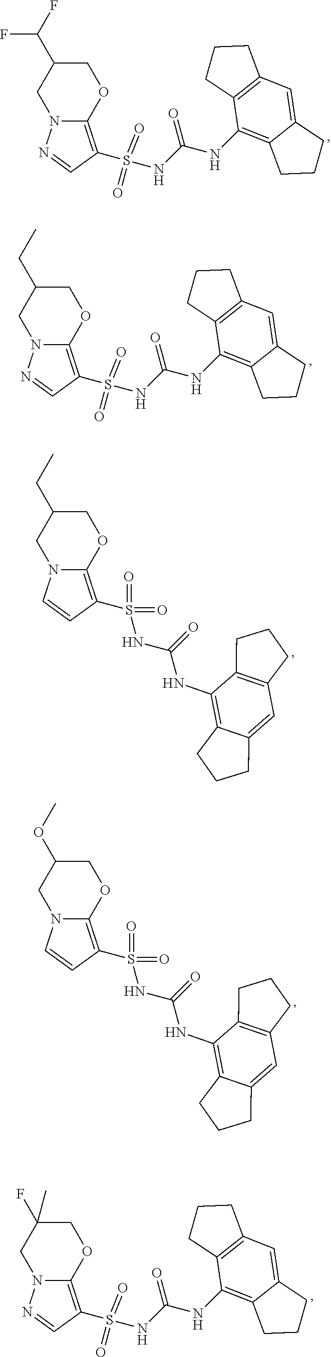 [SMILES: O=C(Nc1c2c(cc3c1CCC3)CCC2)NS(=O)(=O)c1cnn2c1OCC(C(F)F)C2 ; CCC1COc2c(S(=O)(=O)NC(=O)Nc3c4c(cc5c3CCC5)CCC4)cnn2C1 ; CCC1COc2c(S(=O)(=O)NC(=O)Nc3c4c(cc5c3CCC5)CCC4)ccn2C1 ; COC1COc2c(S(=O)(=O)NC(=O)Nc3c4c(cc5c3CCC5)CCC4)ccn2C1 ; CC1(F)COc2c(S(=O)(=O)NC(=O)Nc3c4c(cc5c3CCC5)CCC4)cnn2C1]

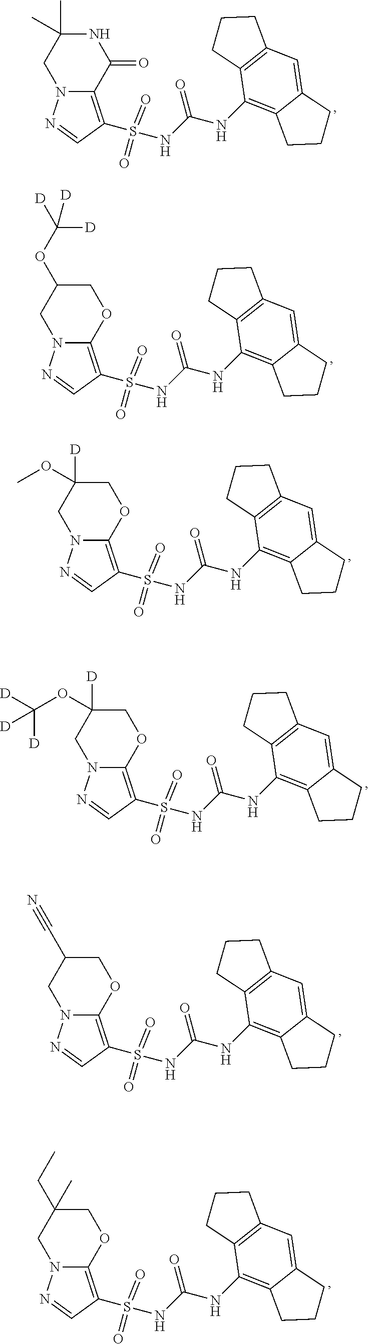 [SMILES: CC1(C)Cn2ncc(S(=O)(=O)NC(=O)Nc3c4c(cc5c3CCC5)CCC4)c2C(=O)N1 ; [2H]C([2H])([2H])OC1COc2c(S(=O)(=O)NC(=O)Nc3c4c(cc5c3CCC5)CCC4)cnn2C1 ; [2H]C1(OC)COc2c(S(=O)(=O)NC(=O)Nc3c4c(cc5c3CCC5)CCC4)cnn2C1 ; [2H]C([2H])([2H])OC1([2H])COc2c(S(=O)(=O)NC(=O)Nc3c4c(cc5c3CCC5)CCC4)cnn2C1 ; N#CC1COc2c(S(=O)(=O)NC(=O)Nc3c4c(cc5c3CCC5)CCC4)cnn2C1 ; CCC1(C)COc2c(S(=O)(=O)NC(=O)Nc3c4c(cc5c3CCC5)CCC4)cnn2C1]

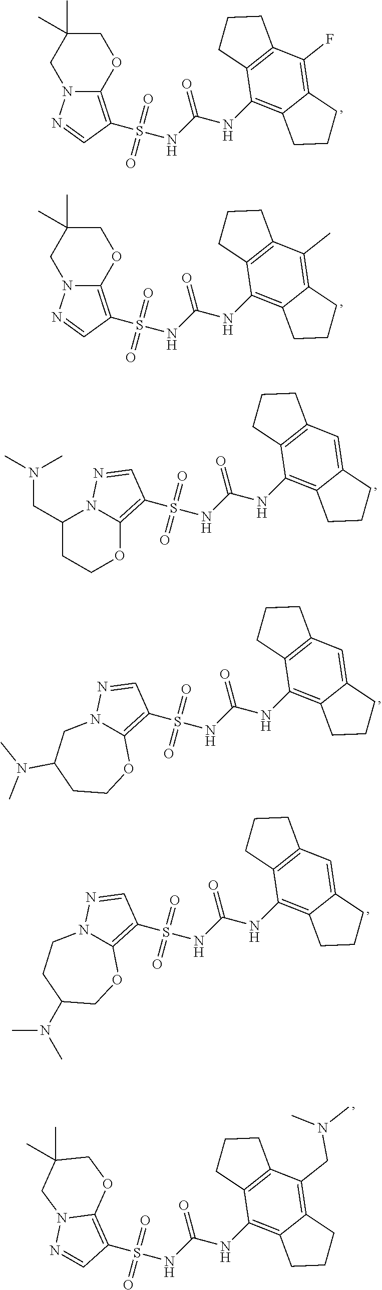

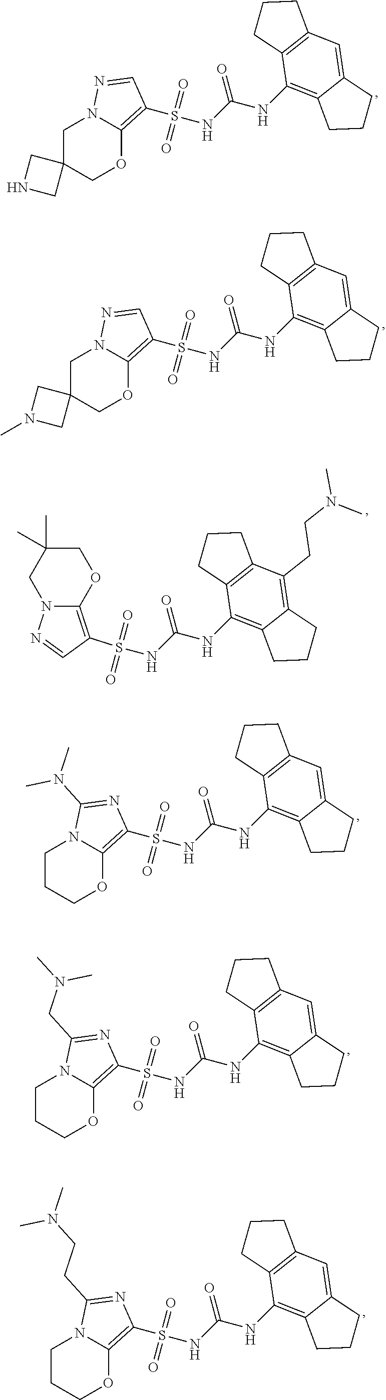

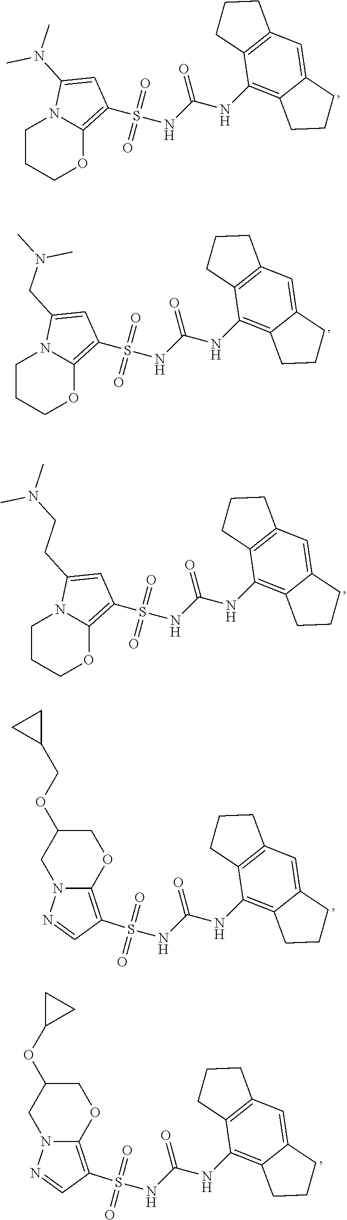

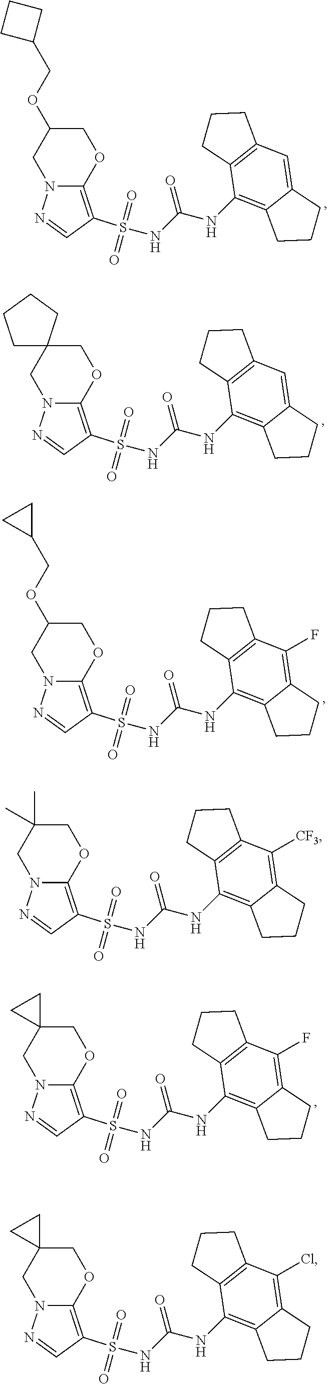 [SMILES: O=C(Nc1c2c(cc3c1CCC3)CCC2)NS(=O)(=O)c1cnn2c1OCC(OCC1CCC1)C2 ; O=C(Nc1c2c(cc3c1CCC3)CCC2)NS(=O)(=O)c1cnn2c1OCC1(CCCC1)C2 ; O=C(Nc1c2c(c(F)c3c1CCC3)CCC2)NS(=O)(=O)c1cnn2c1OCC(OCC1CC1)C2 ; CC1(C)COc2c(S(=O)(=O)NC(=O)Nc3c4c(c(C(F)(F)F)c5c3CCC5)CCC4)cnn2C1 ; O=C(Nc1c2c(c(F)c3c1CCC3)CCC2)NS(=O)(=O)c1cnn2c1OCC1(CC1)C2 ; O=C(Nc1c2c(c(Cl)c3c1CCC3)CCC2)NS(=O)(=O)c1cnn2c1OCC1(CC1)C2]

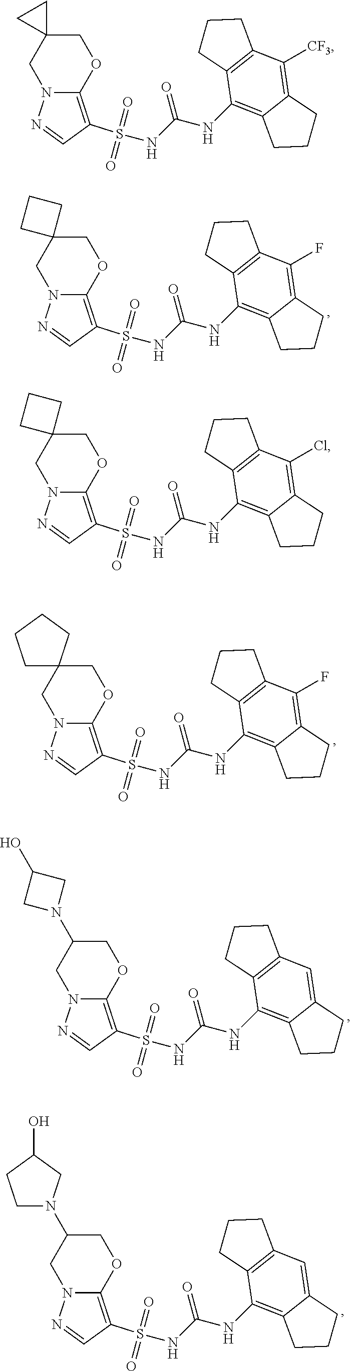 [SMILES: O=C(Nc1c2c(c(C(F)(F)F)c3c1CCC3)CCC2)NS(=O)(=O)c1cnn2c1OCC1(CC1)C2 ; O=C(Nc1c2c(c(F)c3c1CCC3)CCC2)NS(=O)(=O)c1cnn2c1OCC1(CCC1)C2 ; O=C(Nc1c2c(c(Cl)c3c1CCC3)CCC2)NS(=O)(=O)c1cnn2c1OCC1(CCC1)C2 ; O=C(Nc1c2c(c(F)c3c1CCC3)CCC2)NS(=O)(=O)c1cnn2c1OCC1(CCCC1)C2 ; O=C(Nc1c2c(cc3c1CCC3)CCC2)NS(=O)(=O)c1cnn2c1OCC(N1CC(O)C1)C2 ; O=C(Nc1c2c(cc3c1CCC3)CCC2)NS(=O)(=O)c1cnn2c1OCC(N1CCC(O)C1)C2]

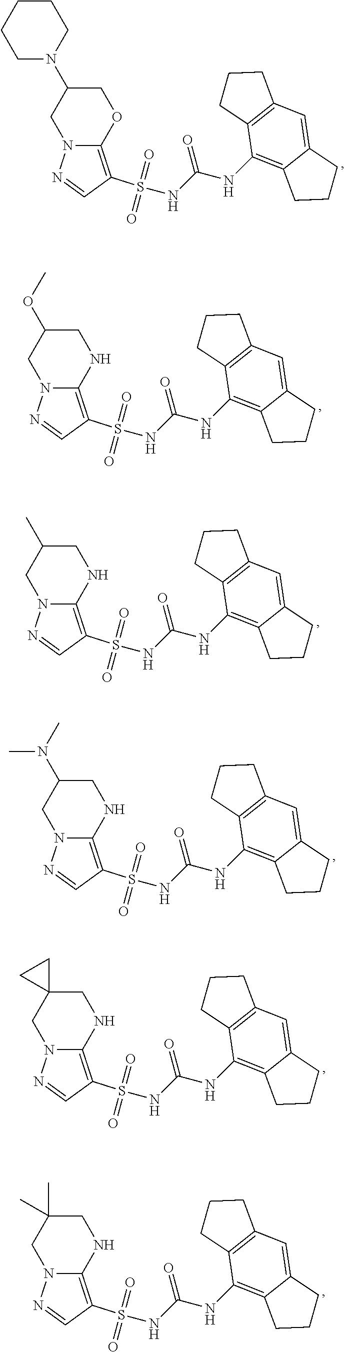 [SMILES: O=C(Nc1c2c(cc3c1CCC3)CCC2)NS(=O)(=O)c1cnn2c1OCC(N1CCCCC1)C2 ; COC1CNc2c(S(=O)(=O)NC(=O)Nc3c4c(cc5c3CCC5)CCC4)cnn2C1 ; CC1CNc2c(S(=O)(=O)NC(=O)Nc3c4c(cc5c3CCC5)CCC4)cnn2C1 ; CN(C)C1CNc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1NCC1(CC1)C2 ; CC1(C)CNc2c(S(=O)(=O)NC(=O)Nc3c4c(cc5c3CCC5)CCC4)cnn2C1]

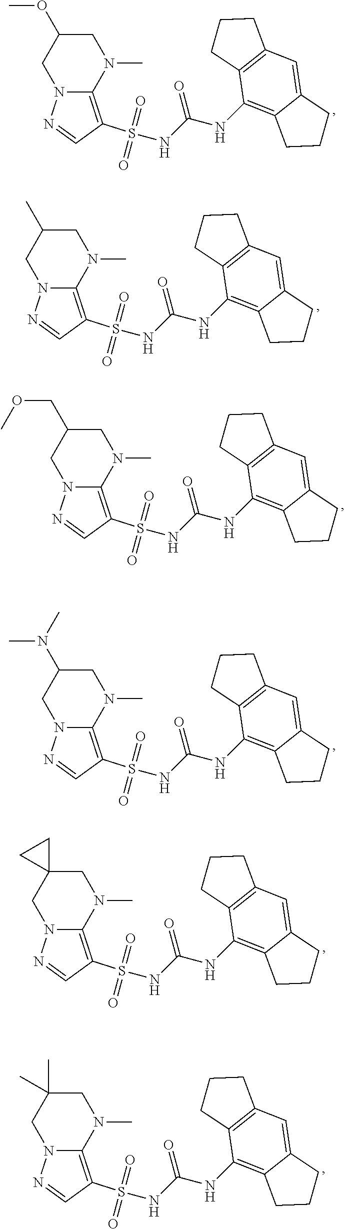 [SMILES: COC1CN(C)c2c(S(=O)(=O)NC(=O)Nc3c4c(cc5c3CCC5)CCC4)cnn2C1 ; CC1CN(C)c2c(S(=O)(=O)NC(=O)Nc3c4c(cc5c3CCC5)CCC4)cnn2C1 ; COCC1CN(C)c2c(S(=O)(=O)NC(=O)Nc3c4c(cc5c3CCC5)CCC4)cnn2C1 ; CN1CC(N(C)C)Cn2ncc(S(=O)(=O)NC(=O)Nc3c4c(cc5c3CCC5)CCC4)c21 ; CN1CC2(CC2)Cn2ncc(S(=O)(=O)NC(=O)Nc3c4c(cc5c3CCC5)CCC4)c21 ; CN1CC(C)(C)Cn2ncc(S(=O)(=O)NC(=O)Nc3c4c(cc5c3CCC5)CCC4)c21]

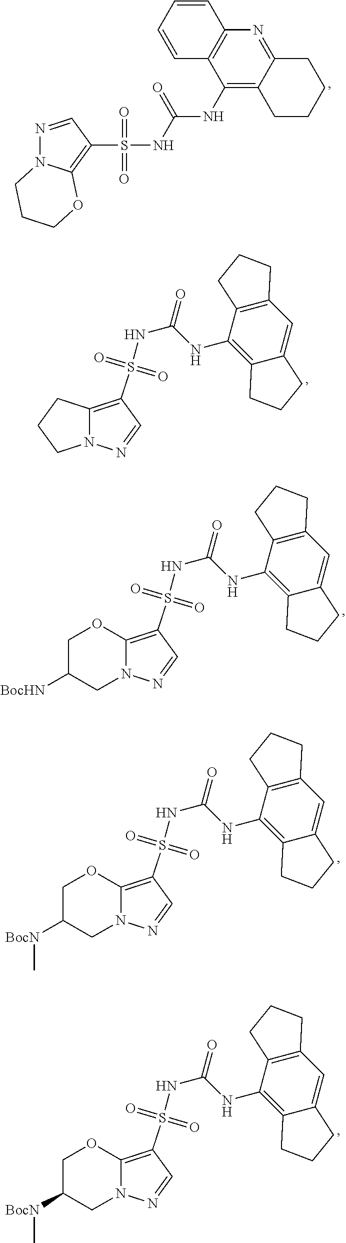 [SMILES: O=C(Nc1c2c(nc3ccccc13)CCCC2)NS(=O)(=O)c1cnn2c1OCCC2 ; O=C(Nc1c2c(cc3c1CCC3)CCC2)NS(=O)(=O)c1cnn2c1CCC2 ; CC(C)(C)OC(=O)NC1COc2c(S(=O)(=O)NC(=O)Nc3c4c(cc5c3CCC5)CCC4)cnn2C1 ; CN(C(=O)OC(C)(C)C)C1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(cc5c3CCC5)CCC4)cnn2C1]

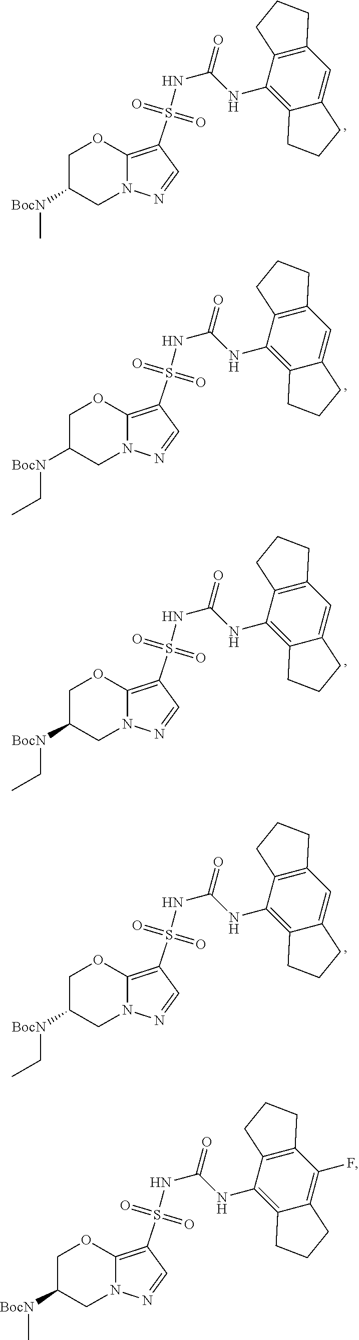 [SMILES: CN(C(=O)OC(C)(C)C)[C@@H]1COc2c(S(=O)(=O)NC(=O)Nc3c4c(cc5c3CCC5)CCC4)cnn2C1 ; CCN(C(=O)OC(C)(C)C)C1COc2c(S(=O)(=O)NC(=O)Nc3c4c(cc5c3CCC5)CCC4)cnn2C1 ; CCN(C(=O)OC(C)(C)C)[C@H]1COc2c(S(=O)(=O)NC(=O)Nc3c4c(cc5c3CCC5)CCC4)cnn2C1 ; CCN(C(=O)OC(C)(C)C)[C@@H]1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(c(F)c5c3CCC5)CCC4)cnn2C1]

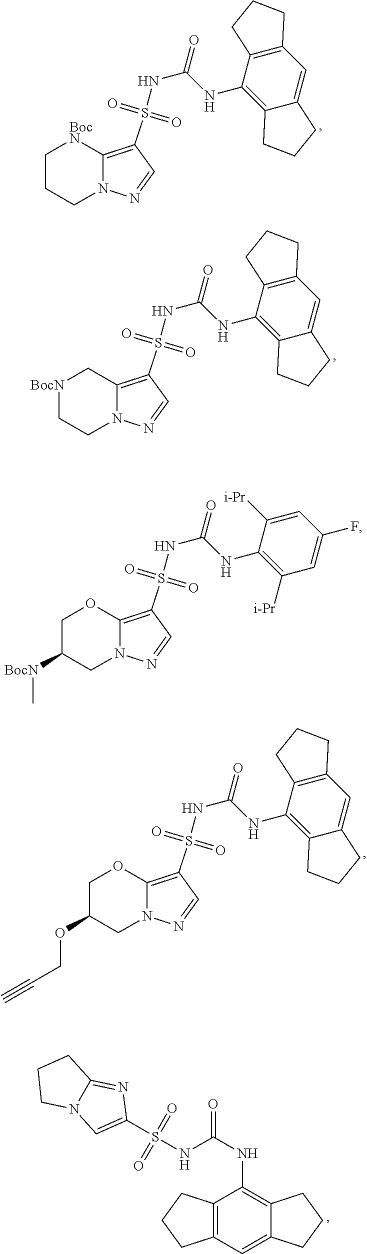 [SMILES: CC(C)(C)OC(=O)N1CCCn2ncc(S(=O)(=O)NC(=O)Nc3c4c(cc5c3CCC5)CCC4)c21 ; CC(C)(C)OC(=O)N1CCn2ncc(S(=O)(=O)NC(=O)Nc3c4c(cc5c3CCC5)CCC4)c2C1 ; CC(C)c1cc(F)cc(C(C)C)c1NC(=O)NS(=O)(=O)c1cnn2c1OC[C@H](N(C)C(=O)OC(C)(C)C)C2 ; C#CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cn2c(n1)CCC2]

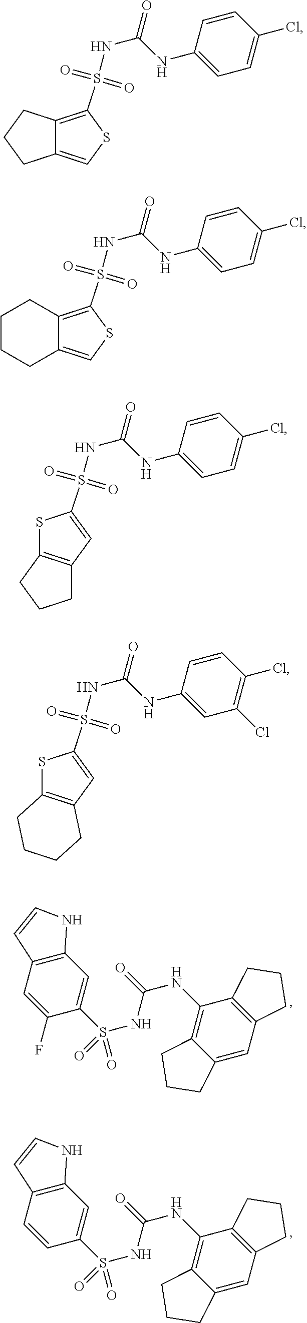 [SMILES: O=C(Nc1ccc(Cl)cc1)NS(=O)(=O)c1scc2c1CCC2 ; O=C(Nc1ccc(Cl)cc1)NS(=O)(=O)c1scc2c1CCCC2 ; O=C(Nc1ccc(Cl)cc1)NS(=O)(=O)c1cc2c(s1)CCC2 ; O=C(Nc1ccc(Cl)c(Cl)c1)NS(=O)(=O)c1cc2c(s1)CCCC2 ; O=C(Nc1c2c(cc3c1CCC3)CCC2)NS(=O)(=O)c1cc2[nH]ccc2cc1F ; O=C(Nc1c2c(cc3c1CCC3)CCC2)NS(=O)(=O)c1ccc2cc[nH]c2c1]

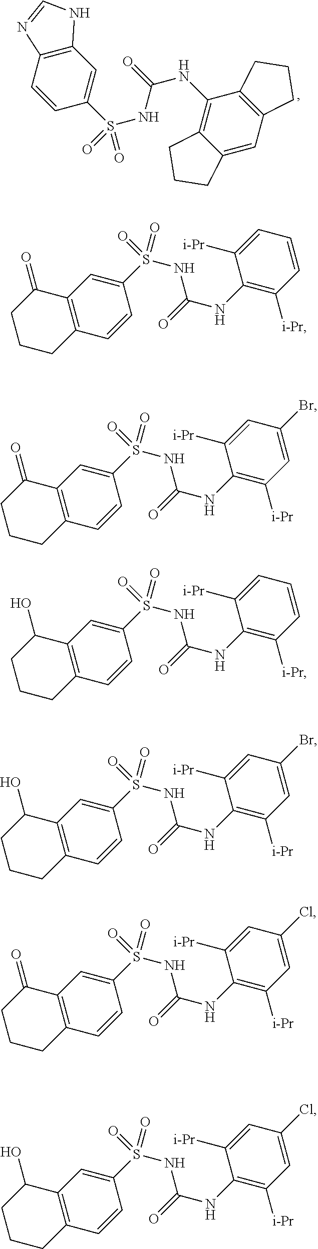

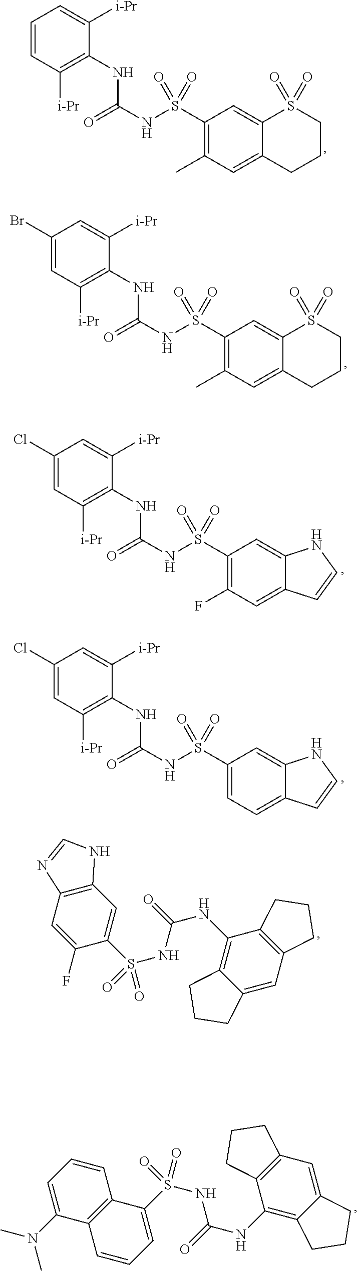 [SMILES: Cc1cc2c(cc1S(=O)(=O)NC(=O)Nc1c(C(C)C)cccc1C(C)C)S(=O)(=O)CCC2 ; Cc1cc2c(cc1S(=O)(=O)NC(=O)Nc1c(C(C)C)cc(Br)cc1C(C)C)S(=O)(=O)CCC2 ; CC(C)c1cc(Cl)cc(C(C)C)c1NC(=O)NS(=O)(=O)c1cc2[nH]ccc2cc1F ; CC(C)c1cc(Cl)cc(C(C)C)c1NC(=O)NS(=O)(=O)c1ccc2cc[nH]c2c1 ; O=C(Nc1c2c(cc3c1CCC3)CCC2)NS(=O)(=O)c1cc2[nH]cnc2cc1F ; CN(C)c1cccc2c(S(=O)(=O)NC(=O)Nc3c4c(cc5c3CCC5)CCC4)cccc12]

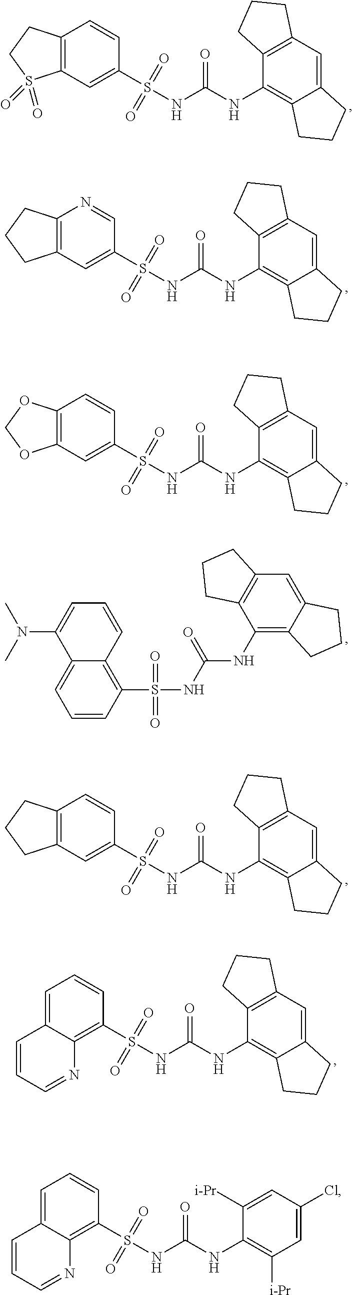

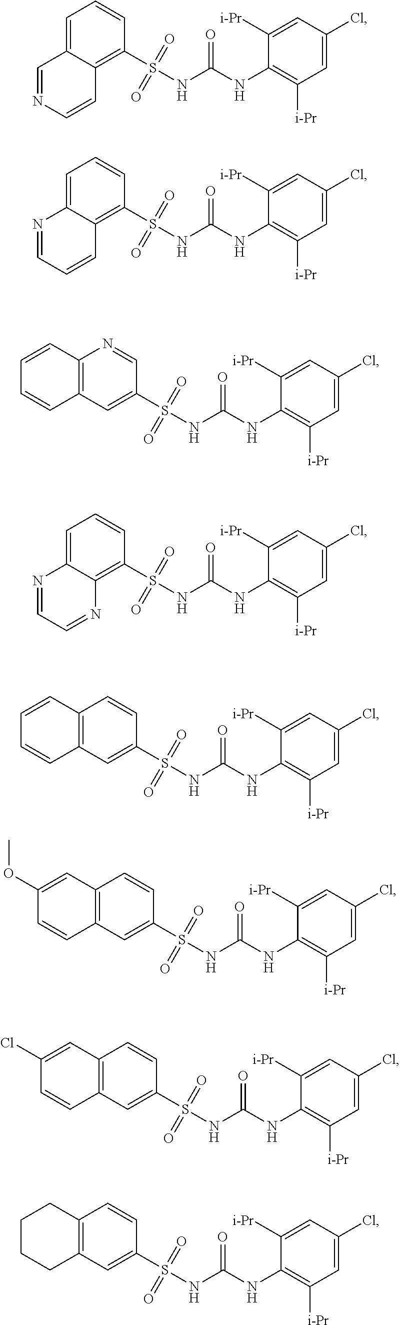

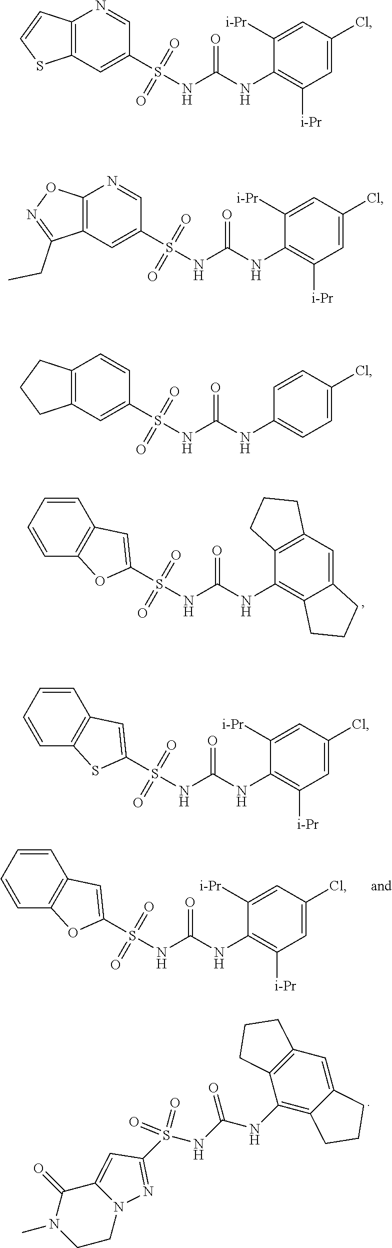

In some embodiments, when B is 5-10-membered heteroaryl including from 2-3 ring nitrogen atoms, at least one R⁶ is attached to B at a position ortho to the —HNC(═O)NHS(O)₂-moiety of Formula AA.

In some embodiments, when B is 2-pyridyl, pyrimidin-6-yl, or pyrimidin-4-yl, B is not substituted with a cyano group at a position ortho to the —HNC(═O)NHS(O)₂— moiety of Formula AA.

In some embodiments, the compound of Formula AA is not selected from the compounds disclosed in WO 2018/136890.

In some embodiments the compound of any of the formulae herein is not a compound disclosed in any of Examples 1-150 of patent publication WO2001/019390, which are incorporated by reference herein.

In some embodiments the compound of any of the formulae herein is not a compound disclosed in patent publication WO2001/019390, which is incorporated by reference herein.

In some embodiments the compound of any of the formulae herein is not a compound disclosed in any of Examples 1-130 of patent publication WO 98/32733, which are incorporated by reference herein.

In some embodiments the compound of any of the formulae herein is not a compound disclosed in patent publication WO 98/32733, which is incorporated by reference herein.

In some embodiments the compound of any of the formulae herein is not a compound disclosed in any of the Examples at [00123] of patent publication WO2016/131098, which are incorporated by reference herein.

In some embodiments the compound of any of the formulae herein is not a compound disclosed in patent publication WO2016/131098, which is incorporated by reference herein.

In some embodiments the compound of any of the formulae herein is not a compound disclosed in DK 2006/00313, which is incorporated by reference herein.

In some embodiments the compound of any of the formulae herein is not a compound disclosed in U.S. Pat. No. 4,927,453, which is incorporated by reference herein.

In some embodiments the compound of any of the formulae herein is not a compound disclosed in EP 03/18620, which is incorporated by reference herein.

In some embodiments the compound of any of the formulae herein is not a compound disclosed in EP 02/05348, which is incorporated by reference herein.

In some embodiments, the compound of any of the formulae herein is not a compound disclosed in J. Med. Chem. 1992, 35, 3012-3016, which is incorporated by reference herein. In some embodiments, the compound of any of the formulae herein is not a compound disclosed in WO 2018/015445 and EP 32/72739, which is incorporated by reference herein.

In some embodiments, the compound of any of the formulae herein is not a compound disclosed in U.S. Pat. No. 5,356,862, which is incorporated by reference herein.

In some embodiments, the compound of any of the formulae herein is not a compound disclosed in WO 91/110668, which is incorporated by reference herein.

In some embodiments, the compound of any of the formulae herein is not a compound disclosed in U.S. Pat. No. 4,671,817, which is incorporated by reference herein.

In some embodiments, the compound of any of the formulae herein is not a compound disclosed in EP 02/38070, which is incorporated by reference herein.

In some embodiments, the compound of any of the formulae herein is not a compound disclosed in EP 15/2286, which is incorporated herein by reference.

In some embodiments the compound of any of the formulae herein is not a compound disclosed in WO2019034686, WO2019034688, WO2019034690, WO2019034692, WO2019034693, WO2019034696, and/or WO2019034697.

In some embodiments, the compound of any of the formulae herein is not a compound disclosed in WO 2018/136890, WO2001/019390, WO 98/32733, WO2016/131098, DK 2006/00313, U.S. Pat. No. 4,927,453, EP 03/18620, EP 02/05348, WO 2018/015445, EP 32/72739, U.S. Pat. No. 5,356,862, WO 91/110668, U.S. Pat. No. 4,671,817, EP 02/38070, EP 15/2286, J Med. Chem. 1992, 35, 3012-3016, WO2019034686, WO2019034688, WO2019034690, WO2019034692, WO2019034693, WO2019034696, and/or WO2019034697.

In some embodiments, the compound is not a compound disclosed in WO 2019/008029, which is incorporated by reference in its entirety.

Unless otherwise indicated, when a disclosed compound is named or depicted by a structure without specifying the stereochemistry and has one or more chiral centers, it is understood to represent all possible stereoisomers of the compound.

In one embodiment, provided herein is a combination of a compound of any preceding embodiment, for use in the treatment or the prevention of a condition mediated by TNF-α, in a patient in need thereof, wherein the compound is administered to said patient at a therapeutically effective amount. Preferably, the subject is resistant to treatment with an anti-TNFα agent. Preferably, the condition is a gut disease or disorder.

In one embodiment, provided herein is a pharmaceutical composition of comprising a compound of any preceding embodiment, and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any preceding embodiment, and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

In one embodiment, the present invention relates to an NLRP3 antagonist for use in the treatment or the prevention of a condition mediated by TNF-α, in particular a gut disease or disorder, in a patient in need thereof, wherein the NLRP3 antagonist is administered to said patient at a therapeutically effective amount.

In one embodiment, the present invention relates to an NLRP3 antagonist for use in the treatment or the prevention of a condition, in particular a gut disease or disorder, in a patient in need thereof wherein the NLRP3 antagonist is administered to said patient at a therapeutically effective amount.

In one embodiment, the present invention relates to an NLRP3 antagonist for use in the treatment, stabilization or lessening the severity or progression of gut disease or disorder, in a patient in need thereof wherein the NLRP3 antagonist is administered to said patient at a therapeutically effective amount.

In one embodiment, the present invention relates to an NLRP3 antagonist for use in the slowing, arresting, or reducing the development of a gut disease or disorder, in a patient in need thereof wherein the NLRP3 antagonist is administered to said patient at a therapeutically effective amount.

In one embodiment, the present invention relates to an NLRP3 antagonist for use according to above listed embodiments wherein the NLRP3 antagonist is a gut-targeted NLRP3 antagonist.

In one embodiment, the present invention relates ton NLRP3 antagonist for use according to any of the above embodiments, wherein the gut disease is IBD.

In one embodiment, the present invention relates to an NLRP3 antagonist for use according to any of the above embodiments, wherein the gut disease is US or CD.

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

In one embodiment, the present invention relates to a method for the treatment or the prevention of a condition, in particular a gut disease or disorder, in a patient in need thereof, comprising administering to said patient a therapeutically effective amount of a gut-targeted NLRP3 antagonist.

In one embodiment, the present invention relates to a method for the treatment, stabilization or lessening the severity or progression of gut disease or disorder, in a patient in need thereof comprising administering to said patient a therapeutically effective amount of a gut-targeted NLRP3 antagonist.

In one embodiment, the present invention relates to a method for slowing, arresting, or reducing the development of a gut disease or disorder, in a patient in need thereof comprising administering to said patient a therapeutically effective amount of a gut-targeted NLRP3 antagonist.

In one embodiment, the present invention relates to a method according to any of the above embodiments, wherein the gut disease is IBD.

In one embodiment, the present invention relates to a method according to any of the above embodiments x to xx, wherein the gut disease is UC or CD.

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

It is understood that the combination of variables in the formulae herein is such that the compounds are stable.

In some embodiments, provided herein is a compound that is selected from the group consisting of the compounds in Table 1A:

TABLE 1A

| Comp. | |
|---|---|
| No. | Structure |
| 1 | 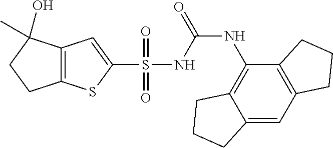 [SMILES: CC1(O)CCc2sc(S(=O)(=O)NC(=O)Nc3c4c(cc5c3CCC5)CCC4)cc21] |
| 2 | 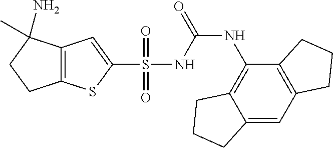 [SMILES: CC1(N)CCc2sc(S(=O)(=O)NC(=O)Nc3c4c(cc5c3CCC5)CCC4)cc21] |
| 3 | 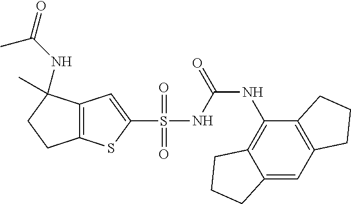 [SMILES: CC(=O)NC1(C)CCc2sc(S(=O)(=O)NC(=O)Nc3c4c(cc5c3CCC5)CCC4)cc21] |
| 4 | 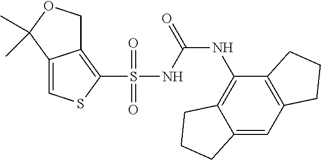 [SMILES: CC1(C)OCc2c1csc2S(=O)(=O)NC(=O)Nc1c2c(cc3c1CCC3)CCC2] |
| 5 | 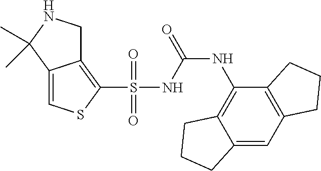 [SMILES: CC1(C)NCc2c1csc2S(=O)(=O)NC(=O)Nc1c2c(cc3c1CCC3)CCC2] |
| 6 | 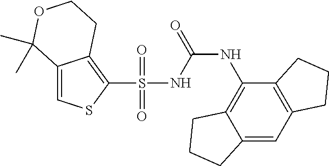 [SMILES: CC1(C)OCCc2c1csc2S(=O)(=O)NC(=O)Nc1c2c(cc3c1CCC3)CCC2] |
| 7 | 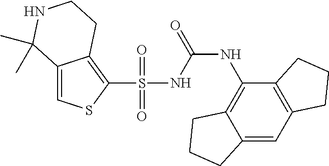 [SMILES: CC1(C)NCCc2c1csc2S(=O)(=O)NC(=O)Nc1c2c(cc3c1CCC3)CCC2] |
| 8 | 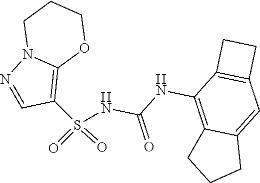 [SMILES: O=C(Nc1c2c(cc3c1CC3)CCC2)NS(=O)(=O)c1cnn2c1OCCC2] |
| 9 | 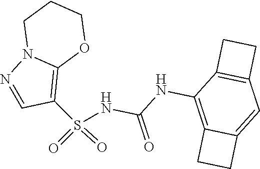 [SMILES: O=C(Nc1c2c(cc3c1CC3)CC2)NS(=O)(=O)c1cnn2c1OCCC2] |
| 10 | 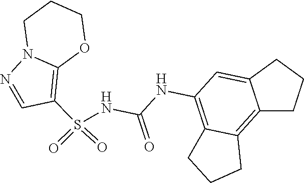 [SMILES: O=C(Nc1cc2c(c3c1CCC3)CCC2)NS(=O)(=O)c1cnn2c1OCCC2] |
| 11 | 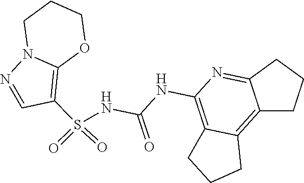 [SMILES: O=C(Nc1nc2c(c3c1CCC3)CCC2)NS(=O)(=O)c1cnn2c1OCCC2] |
| 12 | 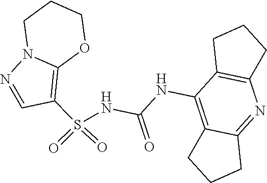 [SMILES: O=C(Nc1c2c(nc3c1CCC3)CCC2)NS(=O)(=O)c1cnn2c1OCCC2] |
| 13 | 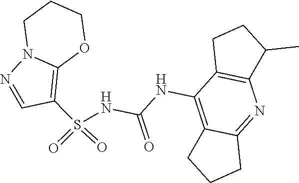 [SMILES: CC1CCc2c1nc1c(c2NC(=O)NS(=O)(=O)c2cnn3c2OCCC3)CCC1] |
| 14 | 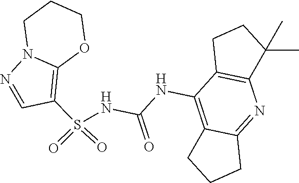 [SMILES: CC1(C)CCc2c1nc1c(c2NC(=O)NS(=O)(=O)c2cnn3c2OCCC3)CCC1] |
| 15 | 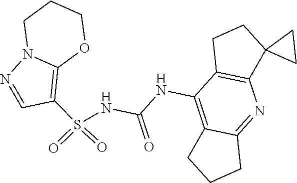 [SMILES: O=C(Nc1c2c(nc3c1CCC31CC1)CCC2)NS(=O)(=O)c1cnn2c1OCCC2] |
| 16 | 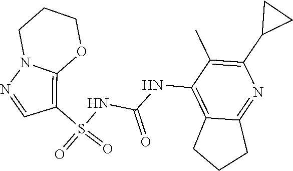 [SMILES: Cc1c(C2CC2)nc2c(c1NC(=O)NS(=O)(=O)c1cnn3c1OCCC3)CCC2] |
| 17 | 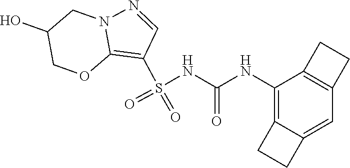 [SMILES: O=C(Nc1c2c(cc3c1CC3)CC2)NS(=O)(=O)c1cnn2c1OCC(O)C2] |
| 18 | 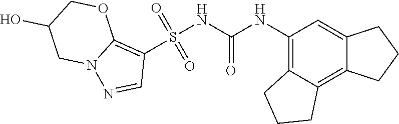 [SMILES: O=C(Nc1cc2c(c3c1CCC3)CCC2)NS(=O)(=O)c1cnn2c1OCC(O)C2] |
| 19 | 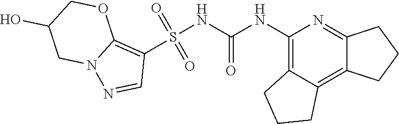 [SMILES: O=C(Nc1nc2c(c3c1CCC3)CCC2)NS(=O)(=O)c1cnn2c1OCC(O)C2] |
| 20 | 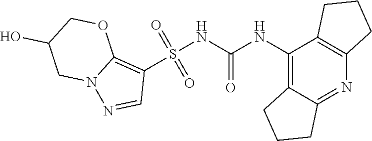 [SMILES: O=C(Nc1c2c(nc3c1CCC3)CCC2)NS(=O)(=O)c1cnn2c1OCC(O)C2] |
| 21 | 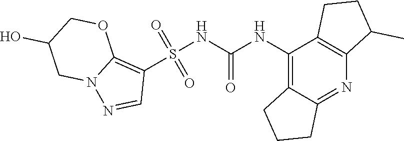 [SMILES: CC1CCc2c1nc1c(c2NC(=O)NS(=O)(=O)c2cnn3c2OCC(O)C3)CCC1] |
| 22 | 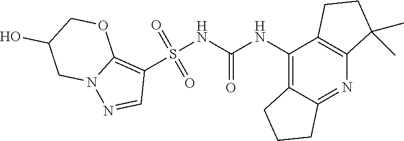 [SMILES: CC1(C)CCc2c1nc1c(c2NC(=O)NS(=O)(=O)c2cnn3c2OCC(O)C3)CCC1] |
| 23 | 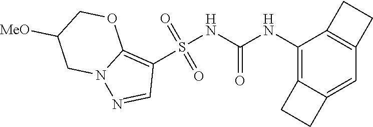 [SMILES: COC1COc2c(S(=O)(=O)NC(=O)Nc3c4c(cc5c3CC5)CC4)cnn2C1] |
| 24 | 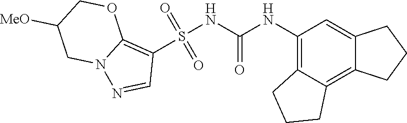 [SMILES: COC1COc2c(S(=O)(=O)NC(=O)Nc3cc4c(c5c3CCC5)CCC4)cnn2C1] |
| 25 | 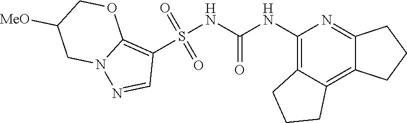 [SMILES: COC1COc2c(S(=O)(=O)NC(=O)Nc3nc4c(c5c3CCC5)CCC4)cnn2C1] |
| 26 | 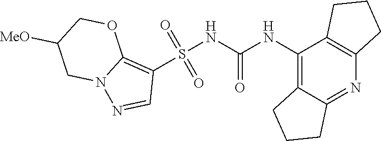 [SMILES: COC1COc2c(S(=O)(=O)NC(=O)Nc3c4c(nc5c3CCC5)CCC4)cnn2C1] |
| 27 | 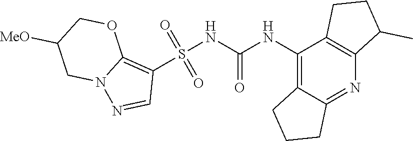 [SMILES: COC1COc2c(S(=O)(=O)NC(=O)Nc3c4c(nc5c3CCC5C)CCC4)cnn2C1] |
| 28 | 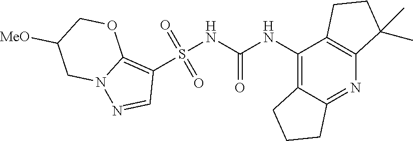 [SMILES: COC1COc2c(S(=O)(=O)NC(=O)Nc3c4c(nc5c3CCC5(C)C)CCC4)cnn2C1] |
| 29 | 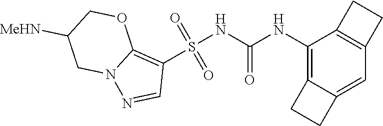 [SMILES: CNC1COc2c(S(=O)(=O)NC(=O)Nc3c4c(cc5c3CC5)CC4)cnn2C1] |
| 30 | 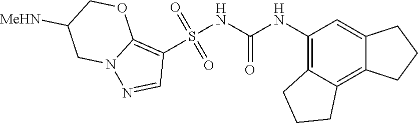 [SMILES: CNC1COc2c(S(=O)(=O)NC(=O)Nc3cc4c(c5c3CCC5)CCC4)cnn2C1] |
| 31 | 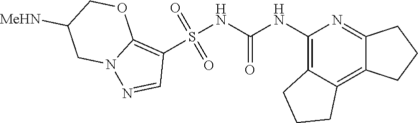 [SMILES: CNC1COc2c(S(=O)(=O)NC(=O)Nc3nc4c(c5c3CCC5)CCC4)cnn2C1] |
| 32 | 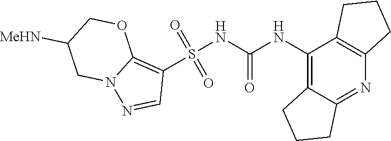 [SMILES: CNC1COc2c(S(=O)(=O)NC(=O)Nc3c4c(nc5c3CCC5)CCC4)cnn2C1] |
| 33 | 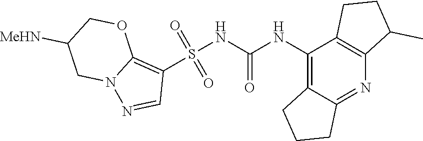 [SMILES: CNC1COc2c(S(=O)(=O)NC(=O)Nc3c4c(nc5c3CCC5C)CCC4)cnn2C1] |
| 34 | 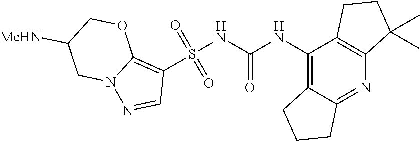 [SMILES: CNC1COc2c(S(=O)(=O)NC(=O)Nc3c4c(nc5c3CCC5(C)C)CCC4)cnn2C1] |
| 35 | 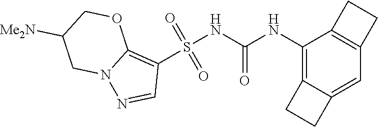 [SMILES: CN(C)C1COc2c(S(=O)(=O)NC(=O)Nc3c4c(cc5c3CC5)CC4)cnn2C1] |
| 36 | 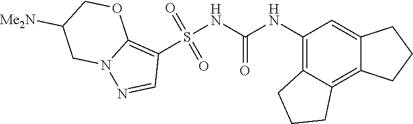 [SMILES: CN(C)C1COc2c(S(=O)(=O)NC(=O)Nc3cc4c(c5c3CCC5)CCC4)cnn2C1] |
| 37 | 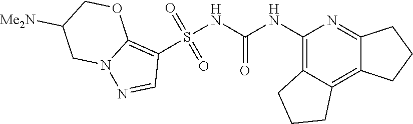 [SMILES: CN(C)C1COc2c(S(=O)(=O)NC(=O)Nc3nc4c(c5c3CCC5)CCC4)cnn2C1] |
| 38 | 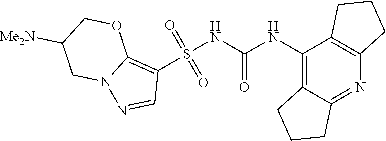 [SMILES: CN(C)C1COc2c(S(=O)(=O)NC(=O)Nc3c4c(nc5c3CCC5)CCC4)cnn2C1] |
| 39 | 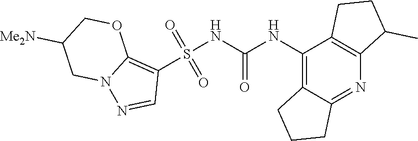 [SMILES: CC1CCc2c1nc1c(c2NC(=O)NS(=O)(=O)c2cnn3c2OCC(N(C)C)C3)CCC1] |
| 40 | 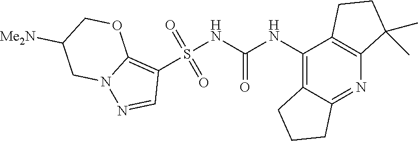 [SMILES: CN(C)C1COc2c(S(=O)(=O)NC(=O)Nc3c4c(nc5c3CCC5(C)C)CCC4)cnn2C1] |
| 41 | 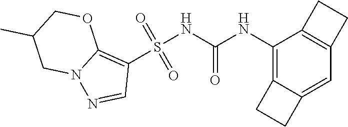 [SMILES: CC1COc2c(S(=O)(=O)NC(=O)Nc3c4c(cc5c3CC5)CC4)cnn2C1] |
| 42 | 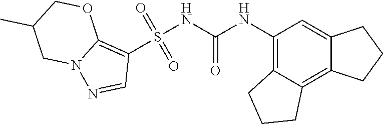 [SMILES: CC1COc2c(S(=O)(=O)NC(=O)Nc3cc4c(c5c3CCC5)CCC4)cnn2C1] |
| 43 | 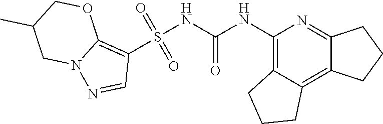 [SMILES: CC1COc2c(S(=O)(=O)NC(=O)Nc3nc4c(c5c3CCC5)CCC4)cnn2C1] |
| 44 | 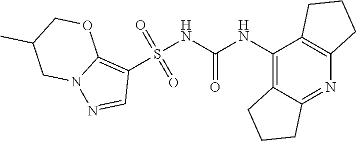 [SMILES: CC1COc2c(S(=O)(=O)NC(=O)Nc3c4c(nc5c3CCC5)CCC4)cnn2C1] |
| 45 | 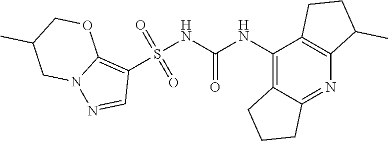 |
| 46 | 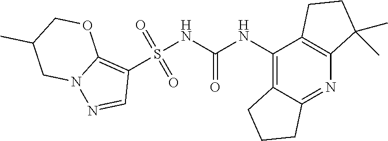 |
| 47 | 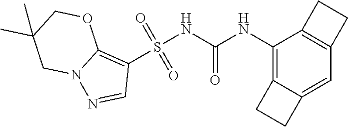 [SMILES: CC1(C)COc2c(S(=O)(=O)NC(=O)Nc3c4c(cc5c3CC5)CC4)cnn2C1] |
| 48 | 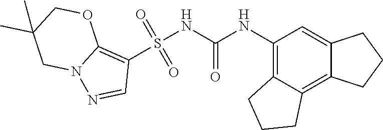 [SMILES: CC1(C)COc2c(S(=O)(=O)NC(=O)Nc3cc4c(c5c3CCC5)CCC4)cnn2C1] |
| 49 | 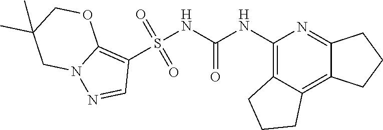 [SMILES: CC1(C)COc2c(S(=O)(=O)NC(=O)Nc3nc4c(c5c3CCC5)CCC4)cnn2C1] |
| 50 | 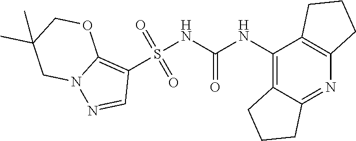 [SMILES: CC1(C)COc2c(S(=O)(=O)NC(=O)Nc3c4c(nc5c3CCC5)CCC4)cnn2C1] |
| 51 | 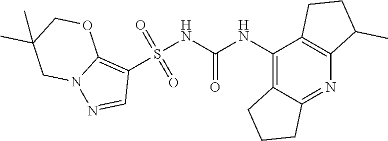 |
| 52 | 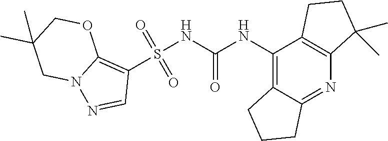 |
| 53 | 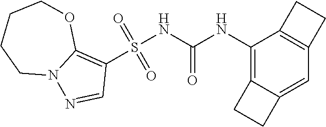 [SMILES: O=C(Nc1c2c(cc3c1CC3)CC2)NS(=O)(=O)c1cnn2c1OCCCC2] |
| 54 | 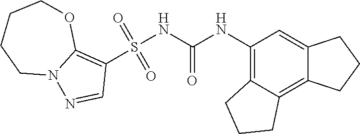 [SMILES: O=C(Nc1cc2c(c3c1CCC3)CCC2)NS(=O)(=O)c1cnn2c1OCCCC2] |
| 55 | 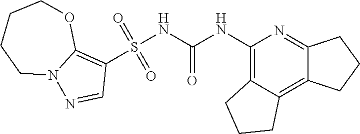 [SMILES: O=C(Nc1nc2c(c3c1CCC3)CCC2)NS(=O)(=O)c1cnn2c1OCCCC2] |
| 56 | 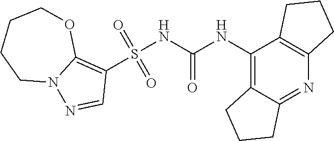 [SMILES: O=C(Nc1c2c(nc3c1CCC3)CCC2)NS(=O)(=O)c1cnn2c1OCCCC2] |
| 57 | 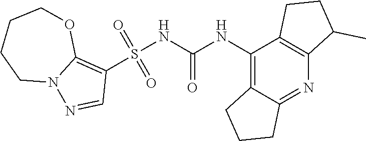 [SMILES: CC1CCc2c1nc1c(c2NC(=O)NS(=O)(=O)c2cnn3c2OCCCC3)CCC1] |
| 58 | 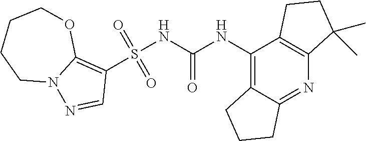 [SMILES: CC1(C)CCc2c1nc1c(c2NC(=O)NS(=O)(=O)c2cnn3c2OCCCC3)CCC1] |
| 59 | 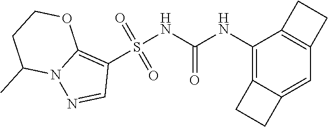 [SMILES: CC1CCOc2c(S(=O)(=O)NC(=O)Nc3c4c(cc5c3CC5)CC4)cnn21] |
| 60 | 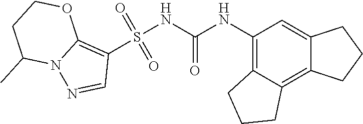 [SMILES: CC1CCOc2c(S(=O)(=O)NC(=O)Nc3cc4c(c5c3CCC5)CCC4)cnn21] |
| 61 | 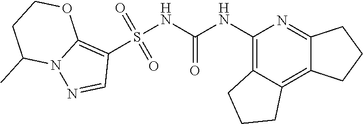 [SMILES: CC1CCOc2c(S(=O)(=O)NC(=O)Nc3nc4c(c5c3CCC5)CCC4)cnn21] |
| 62 | 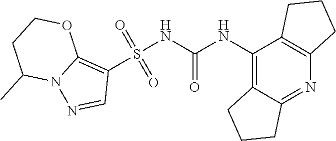 [SMILES: CC1CCOc2c(S(=O)(=O)NC(=O)Nc3c4c(nc5c3CCC5)CCC4)cnn21] |
| 63 | 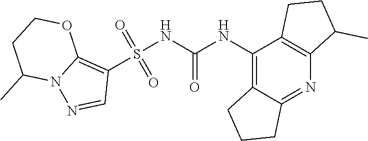 |
| 64 | 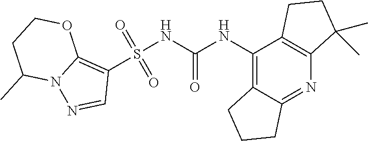 |
| 65 | 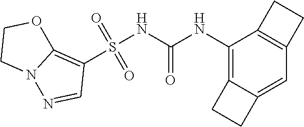 [SMILES: O=C(Nc1c2c(cc3c1CC3)CC2)NS(=O)(=O)c1cnn2c1OCC2] |
| 66 | 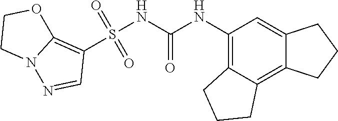 [SMILES: O=C(Nc1cc2c(c3c1CCC3)CCC2)NS(=O)(=O)c1cnn2c1OCC2] |
| 67 | 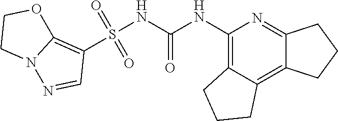 [SMILES: O=C(Nc1nc2c(c3c1CCC3)CCC2)NS(=O)(=O)c1cnn2c1OCC2] |
| 68 | 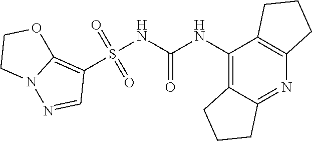 [SMILES: O=C(Nc1c2c(nc3c1CCC3)CCC2)NS(=O)(=O)c1cnn2c1OCC2] |
| 69 | 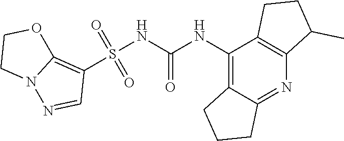 [SMILES: CC1CCc2c1nc1c(c2NC(=O)NS(=O)(=O)c2cnn3c2OCC3)CCC1] |
| 70 | 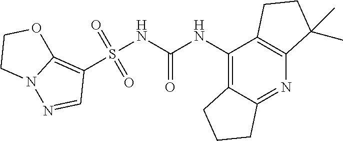 [SMILES: CC1(C)CCc2c1nc1c(c2NC(=O)NS(=O)(=O)c2cnn3c2OCC3)CCC1] |
| 71 | 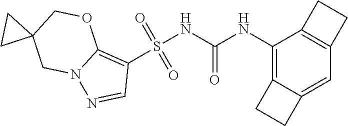 [SMILES: O=C(Nc1c2c(cc3c1CC3)CC2)NS(=O)(=O)c1cnn2c1OCC1(CC1)C2] |
| 72 | 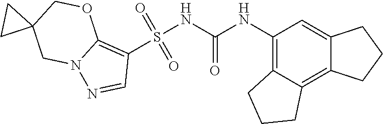 [SMILES: O=C(Nc1cc2c(c3c1CCC3)CCC2)NS(=O)(=O)c1cnn2c1OCC1(CC1)C2] |
| 73 | 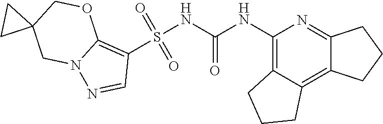 [SMILES: O=C(Nc1nc2c(c3c1CCC3)CCC2)NS(=O)(=O)c1cnn2c1OCC1(CC1)C2] |
| 74 | 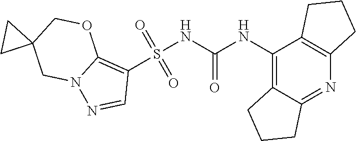 [SMILES: O=C(Nc1c2c(nc3c1CCC3)CCC2)NS(=O)(=O)c1cnn2c1OCC1(CC1)C2] |
| 75 | 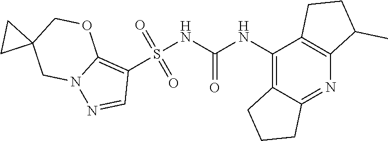 [SMILES: CC1CCc2c1nc1c(c2NC(=O)NS(=O)(=O)c2cnn3c2OCC2(CC2)C3)CCC1] |
| 76 | 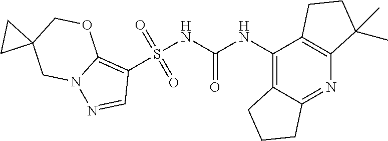 [SMILES: CC1(C)CCc2c1nc1c(c2NC(=O)NS(=O)(=O)c2cnn3c2OCC2(CC2)C3)CCC1] |
| 77 | 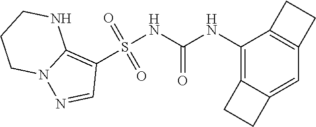 [SMILES: O=C(Nc1c2c(cc3c1CC3)CC2)NS(=O)(=O)c1cnn2c1NCCC2] |
| 78 | 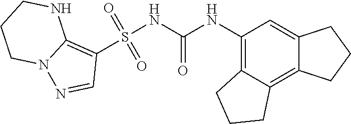 [SMILES: O=C(Nc1cc2c(c3c1CCC3)CCC2)NS(=O)(=O)c1cnn2c1NCCC2] |
| 79 | 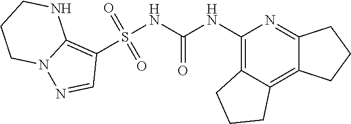 [SMILES: O=C(Nc1nc2c(c3c1CCC3)CCC2)NS(=O)(=O)c1cnn2c1NCCC2] |
| 80 | 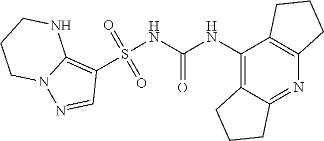 [SMILES: O=C(Nc1c2c(nc3c1CCC3)CCC2)NS(=O)(=O)c1cnn2c1NCCC2] |
| 81 | 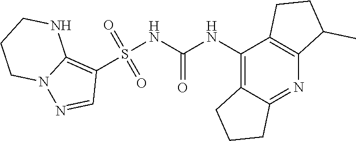 [SMILES: CC1CCc2c1nc1c(c2NC(=O)NS(=O)(=O)c2cnn3c2NCCC3)CCC1] |
| 82 | 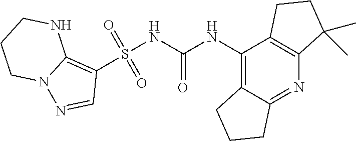 [SMILES: CC1(C)CCc2c1nc1c(c2NC(=O)NS(=O)(=O)c2cnn3c2NCCC3)CCC1] |
| 83 | 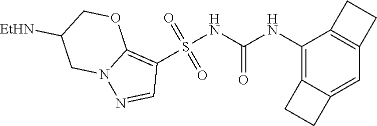 |
| 84 | 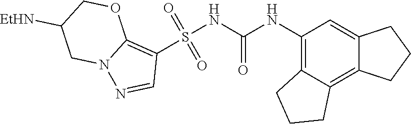 |
| 85 | 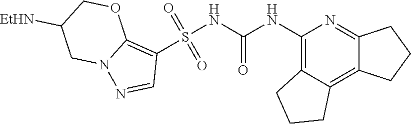 |
| 86 | 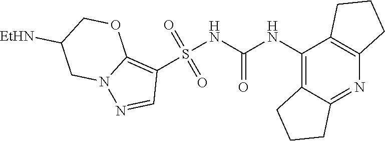 |
| 87 | 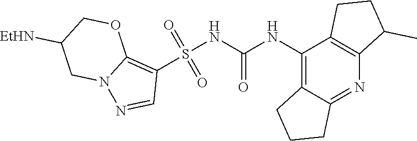 |
| 88 | 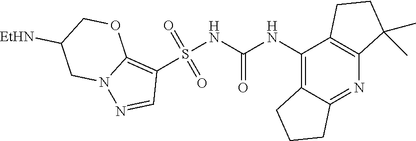 |
| 89 | 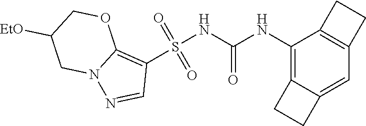 [SMILES: CCOC1COc2c(S(=O)(=O)NC(=O)Nc3c4c(cc5c3CC5)CC4)cnn2C1] |
| 90 | 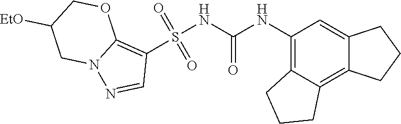 [SMILES: CCOC1COc2c(S(=O)(=O)NC(=O)Nc3cc4c(c5c3CCC5)CCC4)cnn2C1] |
| 91 | 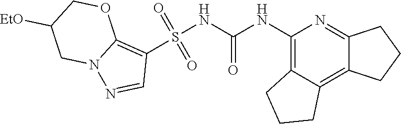 [SMILES: CCOC1COc2c(S(=O)(=O)NC(=O)Nc3nc4c(c5c3CCC5)CCC4)cnn2C1] |
| 92 | 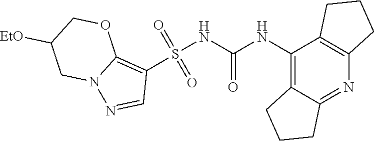 [SMILES: CCOC1COc2c(S(=O)(=O)NC(=O)Nc3c4c(nc5c3CCC5)CCC4)cnn2C1] |
| 93 | 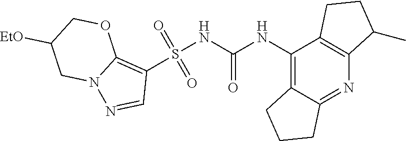 [SMILES: CCOC1COc2c(S(=O)(=O)NC(=O)Nc3c4c(nc5c3CCC5C)CCC4)cnn2C1] |
| 94 | 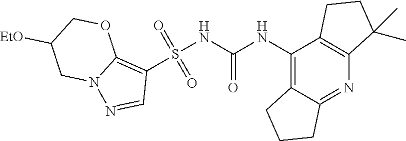 [SMILES: CCOC1COc2c(S(=O)(=O)NC(=O)Nc3c4c(nc5c3CCC5(C)C)CCC4)cnn2C1] |
| 95 | 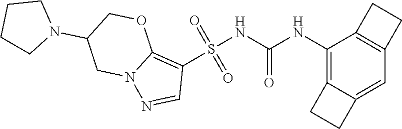 [SMILES: O=C(Nc1c2c(cc3c1CC3)CC2)NS(=O)(=O)c1cnn2c1OCC(N1CCCC1)C2] |
| 96 | 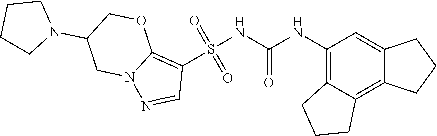 [SMILES: O=C(Nc1cc2c(c3c1CCC3)CCC2)NS(=O)(=O)c1cnn2c1OCC(N1CCCC1)C2] |
| 97 | 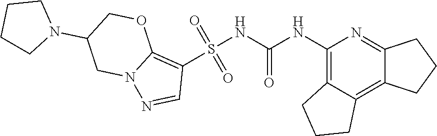 [SMILES: O=C(Nc1nc2c(c3c1CCC3)CCC2)NS(=O)(=O)c1cnn2c1OCC(N1CCCC1)C2] |
| 98 | 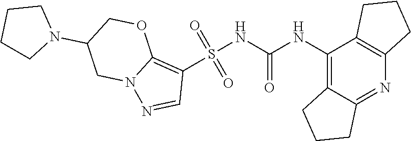 [SMILES: O=C(Nc1c2c(nc3c1CCC3)CCC2)NS(=O)(=O)c1cnn2c1OCC(N1CCCC1)C2] |
| 99 | 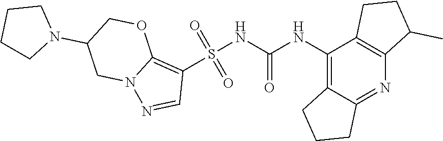 [SMILES: CC1CCc2c1nc1c(c2NC(=O)NS(=O)(=O)c2cnn3c2OCC(N2CCCC2)C3)CCC1] |
| 100 | 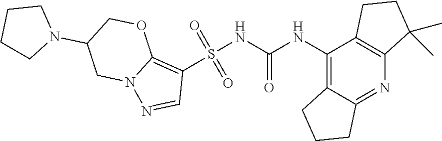 [SMILES: CC1(C)CCc2c1nc1c(c2NC(=O)NS(=O)(=O)c2cnn3c2OCC(N2CCCC2)C3)CCC1] |
| 101 | 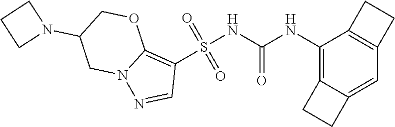 [SMILES: O=C(Nc1c2c(cc3c1CC3)CC2)NS(=O)(=O)c1cnn2c1OCC(N1CCC1)C2] |
| 102 | 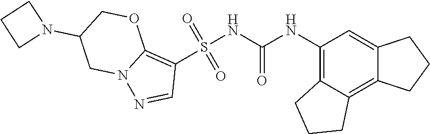 [SMILES: O=C(Nc1cc2c(c3c1CCC3)CCC2)NS(=O)(=O)c1cnn2c1OCC(N1CCC1)C2] |
| 103 | 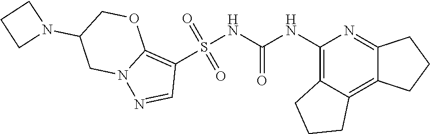 [SMILES: O=C(Nc1nc2c(c3c1CCC3)CCC2)NS(=O)(=O)c1cnn2c1OCC(N1CCC1)C2] |
| 104 | 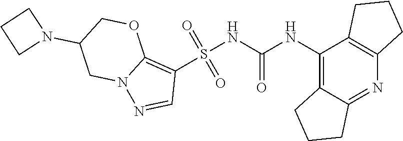 [SMILES: O=C(Nc1c2c(nc3c1CCC3)CCC2)NS(=O)(=O)c1cnn2c1OCC(N1CCC1)C2] |
| 105 | 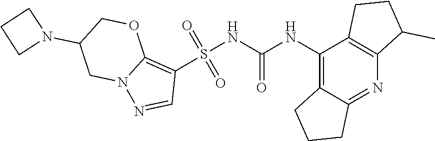 [SMILES: CC1CCc2c1nc1c(c2NC(=O)NS(=O)(=O)c2cnn3c2OCC(N2CCC2)C3)CCC1] |
| 106 | 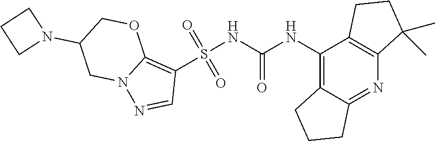 [SMILES: CC1(C)CCc2c1nc1c(c2NC(=O)NS(=O)(=O)c2cnn3c2OCC(N2CCC2)C3)CCC1] |
| 107 | 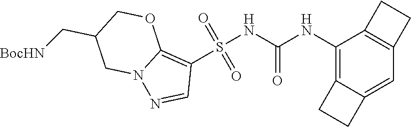 [SMILES: CC(C)(C)OC(=O)NCC1COc2c(S(=O)(=O)NC(=O)Nc3c4c(cc5c3CC5)CC4)cnn2C1] |
| 108 | 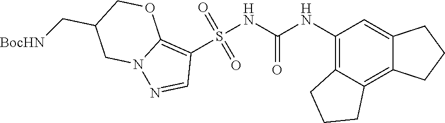 [SMILES: CC(C)(C)OC(=O)NCC1COc2c(S(=O)(=O)NC(=O)Nc3cc4c(c5c3CCC5)CCC4)cnn2C1] |
| 109 | 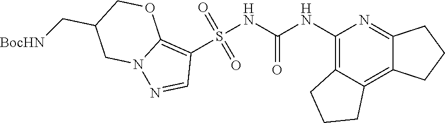 [SMILES: CC(C)(C)OC(=O)NCC1COc2c(S(=O)(=O)NC(=O)Nc3nc4c(c5c3CCC5)CCC4)cnn2C1] |
| 110 | 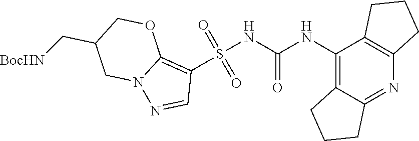 [SMILES: CC(C)(C)OC(=O)NCC1COc2c(S(=O)(=O)NC(=O)Nc3c4c(nc5c3CCC5)CCC4)cnn2C1] |
| 111 | 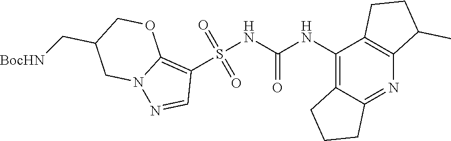 [SMILES: CC1CCc2c1nc1c(c2NC(=O)NS(=O)(=O)c2cnn3c2OCC(CNC(=O)OC(C)(C)C)C3)CCC1] |
| 112 | 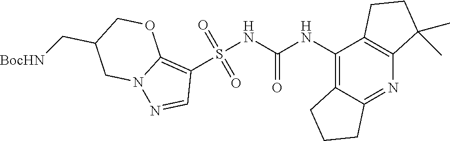 [SMILES: CC(C)(C)OC(=O)NCC1COc2c(S(=O)(=O)NC(=O)Nc3c4c(nc5c3CCC5(C)C)CCC4)cnn2C1] |
| 113 | 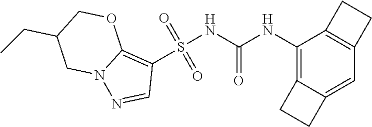 [SMILES: CCC1COc2c(S(=O)(=O)NC(=O)Nc3c4c(cc5c3CC5)CC4)cnn2C1] |
| 114 | 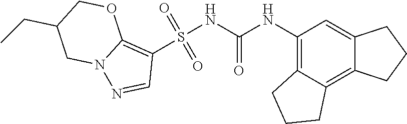 [SMILES: CCC1COc2c(S(=O)(=O)NC(=O)Nc3cc4c(c5c3CCC5)CCC4)cnn2C1] |
| 115 | 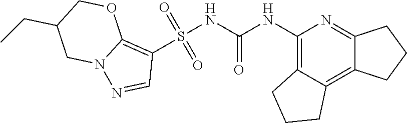 [SMILES: CCC1COc2c(S(=O)(=O)NC(=O)Nc3nc4c(c5c3CCC5)CCC4)cnn2C1] |
| 116 | 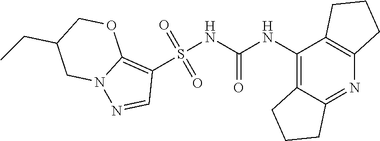 [SMILES: CCC1COc2c(S(=O)(=O)NC(=O)Nc3c4c(nc5c3CCC5)CCC4)cnn2C1] |
| 117 | 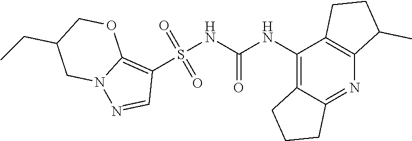 [SMILES: CCC1COc2c(S(=O)(=O)NC(=O)Nc3c4c(nc5c3CCC5C)CCC4)cnn2C1] |
| 118 | 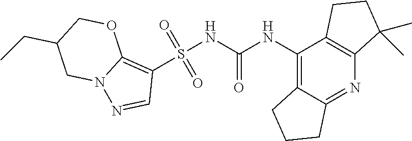 [SMILES: CCC1COc2c(S(=O)(=O)NC(=O)Nc3c4c(nc5c3CCC5(C)C)CCC4)cnn2C1] |
| 119 | 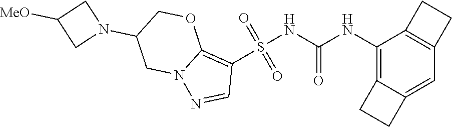 [SMILES: COC1CN(C2COc3c(S(=O)(=O)NC(=O)Nc4c5c(cc6c4CC6)CC5)cnn3C2)C1] |
| 120 | 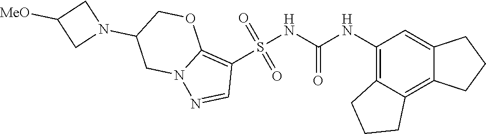 [SMILES: COC1CN(C2COc3c(S(=O)(=O)NC(=O)Nc4cc5c(c6c4CCC6)CCC5)cnn3C2)C1] |
| 121 | 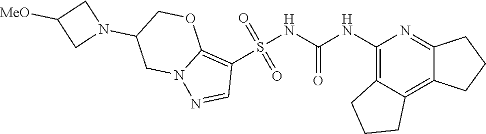 [SMILES: COC1CN(C2COc3c(S(=O)(=O)NC(=O)Nc4nc5c(c6c4CCC6)CCC5)cnn3C2)C1] |
| 122 | 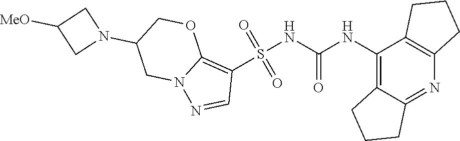 [SMILES: COC1CN(C2COc3c(S(=O)(=O)NC(=O)Nc4c5c(nc6c4CCC6)CCC5)cnn3C2)C1] |
| 123 | 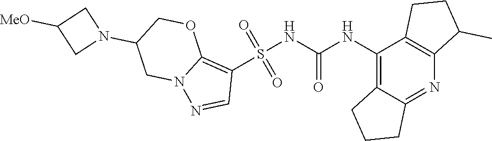 [SMILES: COC1CN(C2COc3c(S(=O)(=O)NC(=O)Nc4c5c(nc6c4CCC6C)CCC5)cnn3C2)C1] |
| 124 | 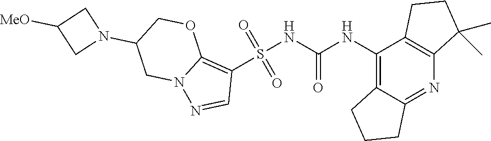 [SMILES: COC1CN(C2COc3c(S(=O)(=O)NC(=O)Nc4c5c(nc6c4CCC6(C)C)CCC5)cnn3C2)C1] |
| 125 | 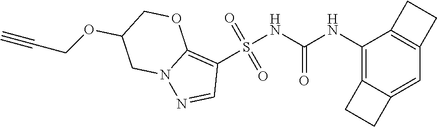 [SMILES: C#CCOC1COc2c(S(=O)(=O)NC(=O)Nc3c4c(cc5c3CC5)CC4)cnn2C1] |
| 126 | 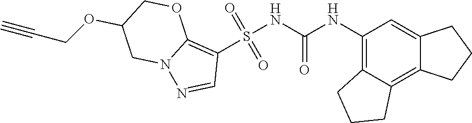 [SMILES: C#CCOC1COc2c(S(=O)(=O)NC(=O)Nc3cc4c(c5c3CCC5)CCC4)cnn2C1] |
| 127 | 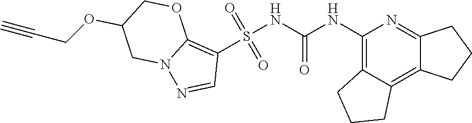 [SMILES: C#CCOC1COc2c(S(=O)(=O)NC(=O)Nc3nc4c(c5c3CCC5)CCC4)cnn2C1] |
| 128 | 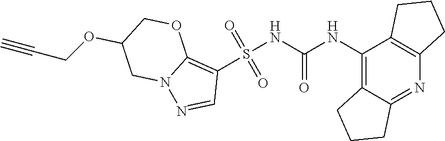 [SMILES: C#CCOC1COc2c(S(=O)(=O)NC(=O)Nc3c4c(nc5c3CCC5)CCC4)cnn2C1] |
| 129 | 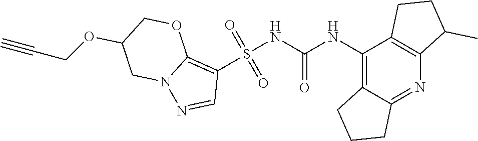 [SMILES: C#CCOC1COc2c(S(=O)(=O)NC(=O)Nc3c4c(nc5c3CCC5C)CCC4)cnn2C1] |
| 130 | 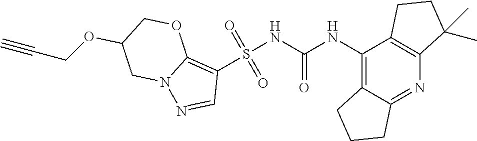 [SMILES: C#CCOC1COc2c(S(=O)(=O)NC(=O)Nc3c4c(nc5c3CCC5(C)C)CCC4)cnn2C1] |
| 131 | 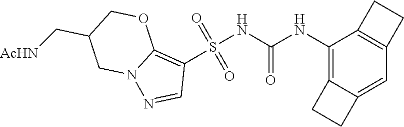 [SMILES: CC(=O)NCC1COc2c(S(=O)(=O)NC(=O)Nc3c4c(cc5c3CC5)CC4)cnn2C1] |
| 132 | 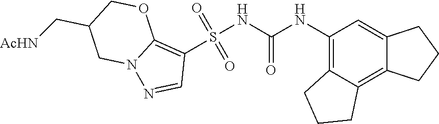 [SMILES: CC(=O)NCC1COc2c(S(=O)(=O)NC(=O)Nc3cc4c(c5c3CCC5)CCC4)cnn2C1] |
| 133 | 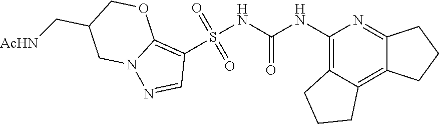 [SMILES: CC(=O)NCC1COc2c(S(=O)(=O)NC(=O)Nc3nc4c(c5c3CCC5)CCC4)cnn2C1] |
| 134 | 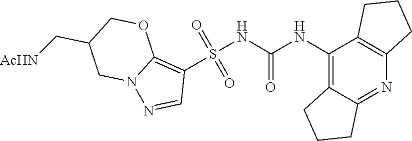 [SMILES: CC(=O)NCC1COc2c(S(=O)(=O)NC(=O)Nc3c4c(nc5c3CCC5)CCC4)cnn2C1] |
| 135 | 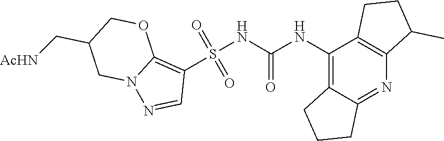 [SMILES: CC(=O)NCC1COc2c(S(=O)(=O)NC(=O)Nc3c4c(nc5c3CCC5C)CCC4)cnn2C1] |
| 136 | 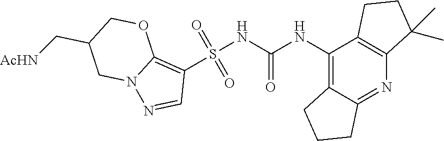 [SMILES: CC(=O)NCC1COc2c(S(=O)(=O)NC(=O)Nc3c4c(nc5c3CCC5(C)C)CCC4)cnn2C1] |
| 137 | 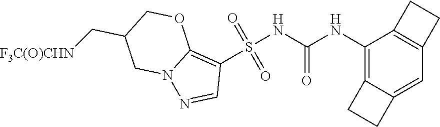 |
| 138 | 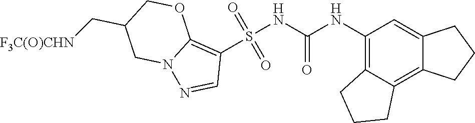 |
| 139 | 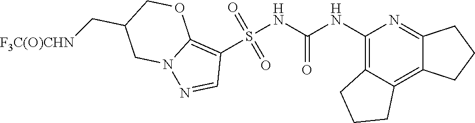 |
| 140 | 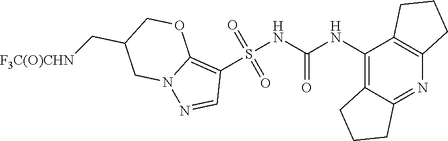 |
| 141 | 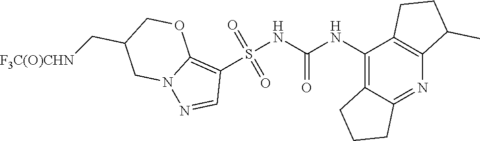 |
| 142 | 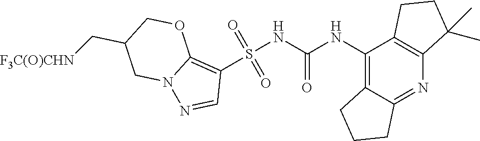 |
| 143 | 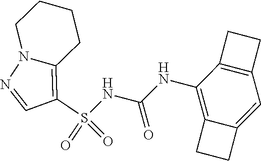 [SMILES: O=C(Nc1c2c(cc3c1CC3)CC2)NS(=O)(=O)c1cnn2c1CCCC2] |
| 144 | 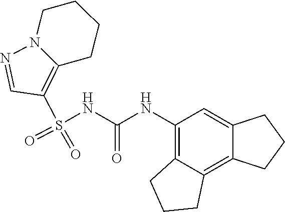 [SMILES: O=C(Nc1cc2c(c3c1CCC3)CCC2)NS(=O)(=O)c1cnn2c1CCCC2] |
| 145 | 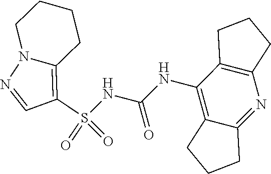 [SMILES: O=C(Nc1c2c(nc3c1CCC3)CCC2)NS(=O)(=O)c1cnn2c1CCCC2] |
| 146 | 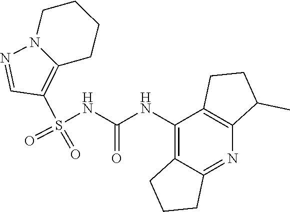 [SMILES: CC1CCc2c1nc1c(c2NC(=O)NS(=O)(=O)c2cnn3c2CCCC3)CCC1] |
| 147 | 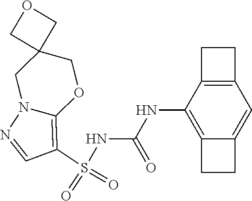 [SMILES: O=C(Nc1c2c(cc3c1CC3)CC2)NS(=O)(=O)c1cnn2c1OCC1(COC1)C2] |
| 148 | 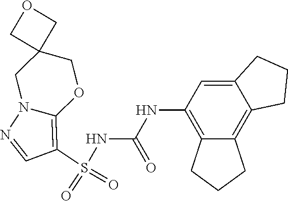 [SMILES: O=C(Nc1cc2c(c3c1CCC3)CCC2)NS(=O)(=O)c1cnn2c1OCC1(COC1)C2] |
| 149 | 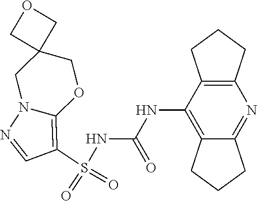 [SMILES: O=C(Nc1c2c(nc3c1CCC3)CCC2)NS(=O)(=O)c1cnn2c1OCC1(COC1)C2] |
| 150 | 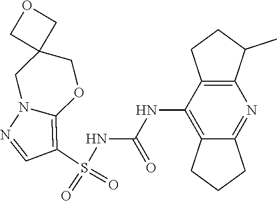 [SMILES: CC1CCc2c1nc1c(c2NC(=O)NS(=O)(=O)c2cnn3c2OCC2(COC2)C3)CCC1] |
| 151 | 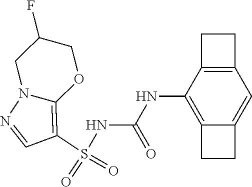 [SMILES: O=C(Nc1c2c(cc3c1CC3)CC2)NS(=O)(=O)c1cnn2c1OCC(F)C2] |
| 152 | 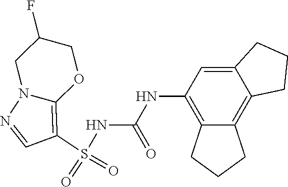 [SMILES: O=C(Nc1cc2c(c3c1CCC3)CCC2)NS(=O)(=O)c1cnn2c1OCC(F)C2] |
| 153 | 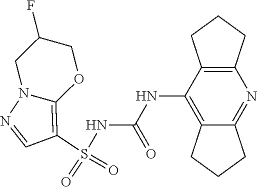 [SMILES: O=C(Nc1c2c(nc3c1CCC3)CCC2)NS(=O)(=O)c1cnn2c1OCC(F)C2] |
| 154 | 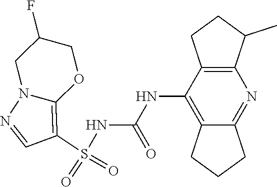 [SMILES: CC1CCc2c1nc1c(c2NC(=O)NS(=O)(=O)c2cnn3c2OCC(F)C3)CCC1] |
| 155 | 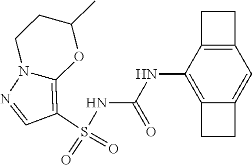 [SMILES: CC1CCn2ncc(S(=O)(=O)NC(=O)Nc3c4c(cc5c3CC5)CC4)c2O1] |
| 156 | 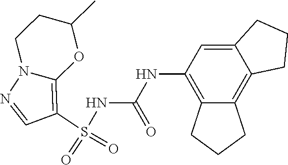 [SMILES: CC1CCn2ncc(S(=O)(=O)NC(=O)Nc3cc4c(c5c3CCC5)CCC4)c2O1] |
| 157 | 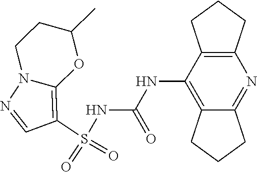 [SMILES: CC1CCn2ncc(S(=O)(=O)NC(=O)Nc3c4c(nc5c3CCC5)CCC4)c2O1] |
| 158 | 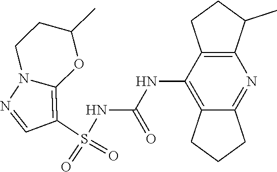 [SMILES: CC1CCn2ncc(S(=O)(=O)NC(=O)Nc3c4c(nc5c3CCC5C)CCC4)c2O1] |
| 159 | 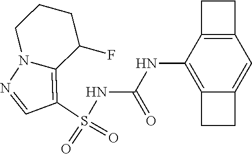 [SMILES: O=C(Nc1c2c(cc3c1CC3)CC2)NS(=O)(=O)c1cnn2c1C(F)CCC2] |
| 160 | 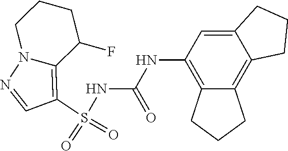 [SMILES: O=C(Nc1cc2c(c3c1CCC3)CCC2)NS(=O)(=O)c1cnn2c1C(F)CCC2] |
| 161 | 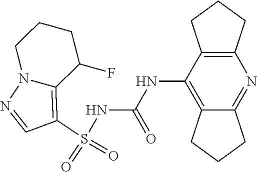 [SMILES: O=C(Nc1c2c(nc3c1CCC3)CCC2)NS(=O)(=O)c1cnn2c1C(F)CCC2] |
| 162 | 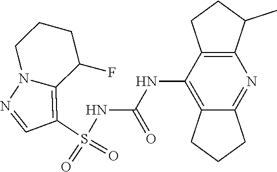 [SMILES: CC1CCc2c1nc1c(c2NC(=O)NS(=O)(=O)c2cnn3c2C(F)CCC3)CCC1] |
| 163 | 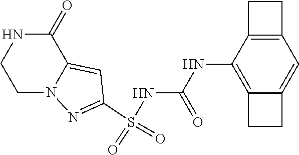 [SMILES: O=C(Nc1c2c(cc3c1CC3)CC2)NS(=O)(=O)c1cc2n(n1)CCNC2=O] |
| 164 | 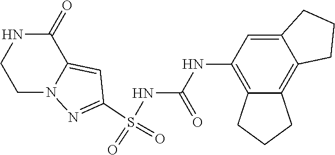 [SMILES: O=C(Nc1cc2c(c3c1CCC3)CCC2)NS(=O)(=O)c1cc2n(n1)CCNC2=O] |
| 165 | 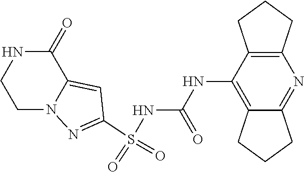 [SMILES: O=C(Nc1c2c(nc3c1CCC3)CCC2)NS(=O)(=O)c1cc2n(n1)CCNC2=O] |
| 166 | 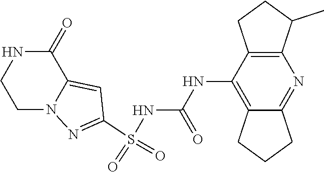 [SMILES: CC1CCc2c1nc1c(c2NC(=O)NS(=O)(=O)c2cc3n(n2)CCNC3=O)CCC1] |
| 167 | 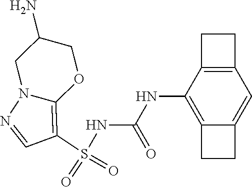 [SMILES: NC1COc2c(S(=O)(=O)NC(=O)Nc3c4c(cc5c3CC5)CC4)cnn2C1] |
| 168 | 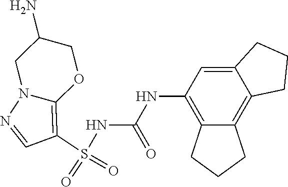 [SMILES: NC1COc2c(S(=O)(=O)NC(=O)Nc3cc4c(c5c3CCC5)CCC4)cnn2C1] |
| 169 | 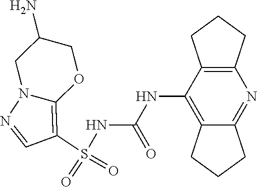 [SMILES: NC1COc2c(S(=O)(=O)NC(=O)Nc3c4c(nc5c3CCC5)CCC4)cnn2C1] |
| 170 | 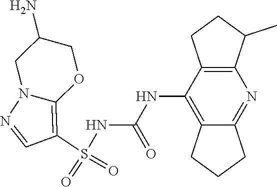 [SMILES: CC1CCc2c1nc1c(c2NC(=O)NS(=O)(=O)c2cnn3c2OCC(N)C3)CCC1] |
| 171 | 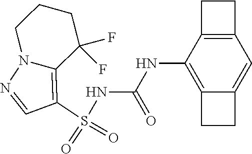 [SMILES: O=C(Nc1c2c(cc3c1CC3)CC2)NS(=O)(=O)c1cnn2c1C(F)(F)CCC2] |
| 172 | 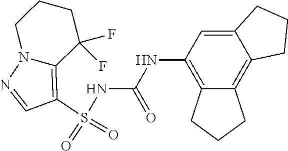 [SMILES: O=C(Nc1cc2c(c3c1CCC3)CCC2)NS(=O)(=O)c1cnn2c1C(F)(F)CCC2] |
| 173 | 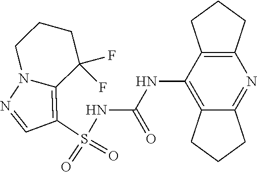 [SMILES: O=C(Nc1c2c(nc3c1CCC3)CCC2)NS(=O)(=O)c1cnn2c1C(F)(F)CCC2] |
| 174 | 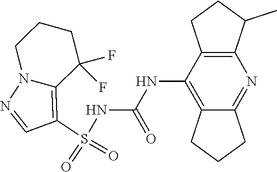 [SMILES: CC1CCc2c1nc1c(c2NC(=O)NS(=O)(=O)c2cnn3c2C(F)(F)CCC3)CCC1] |
| 175 | 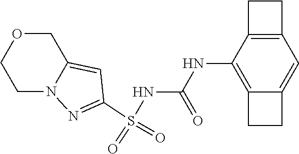 [SMILES: O=C(Nc1c2c(cc3c1CC3)CC2)NS(=O)(=O)c1cc2n(n1)CCOC2] |
| 176 | 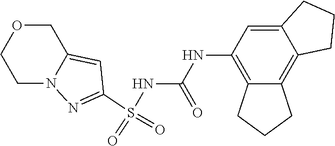 [SMILES: O=C(Nc1cc2c(c3c1CCC3)CCC2)NS(=O)(=O)c1cc2n(n1)CCOC2] |
| 177 | 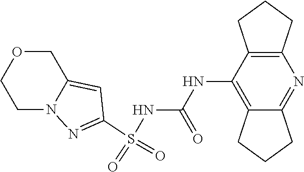 [SMILES: O=C(Nc1c2c(nc3c1CCC3)CCC2)NS(=O)(=O)c1cc2n(n1)CCOC2] |
| 178 | 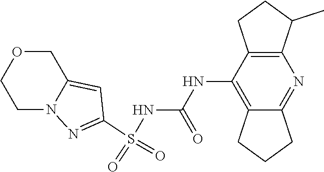 [SMILES: CC1CCc2c1nc1c(c2NC(=O)NS(=O)(=O)c2cc3n(n2)CCOC3)CCC1] |
| 179 | 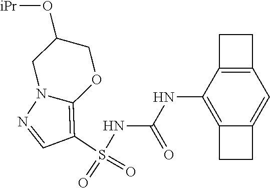 [SMILES: CC(C)OC1COc2c(S(=O)(=O)NC(=O)Nc3c4c(cc5c3CC5)CC4)cnn2C1] |
| 180 | 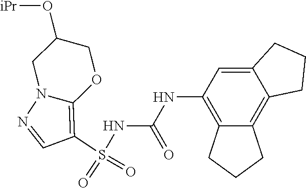 [SMILES: CC(C)OC1COc2c(S(=O)(=O)NC(=O)Nc3cc4c(c5c3CCC5)CCC4)cnn2C1] |
| 181 | 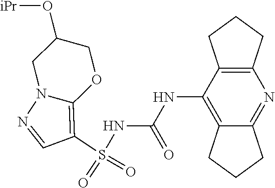 [SMILES: CC(C)OC1COc2c(S(=O)(=O)NC(=O)Nc3c4c(nc5c3CCC5)CCC4)cnn2C1] |
| 182 | 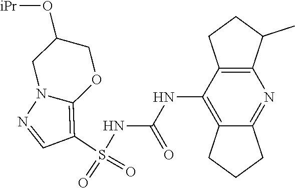 [SMILES: CC(C)OC1COc2c(S(=O)(=O)NC(=O)Nc3c4c(nc5c3CCC5C)CCC4)cnn2C1] |
| 183 | 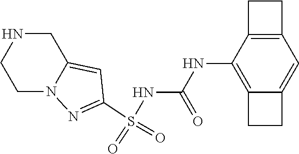 [SMILES: O=C(Nc1c2c(cc3c1CC3)CC2)NS(=O)(=O)c1cc2n(n1)CCNC2] |
| 184 | 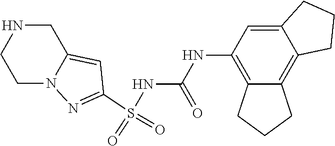 [SMILES: O=C(Nc1cc2c(c3c1CCC3)CCC2)NS(=O)(=O)c1cc2n(n1)CCNC2] |
| 185 | 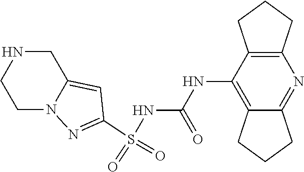 [SMILES: O=C(Nc1c2c(nc3c1CCC3)CCC2)NS(=O)(=O)c1cc2n(n1)CCNC2] |
| 186 | 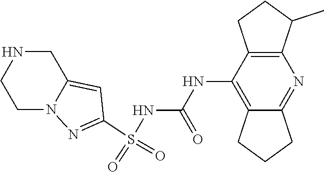 [SMILES: CC1CCc2c1nc1c(c2NC(=O)NS(=O)(=O)c2cc3n(n2)CCNC3)CCC1] |
| 187 | 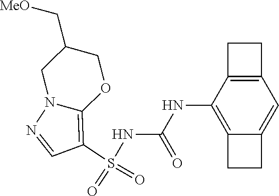 [SMILES: COCC1COc2c(S(=O)(=O)NC(=O)Nc3c4c(cc5c3CC5)CC4)cnn2C1] |
| 188 | 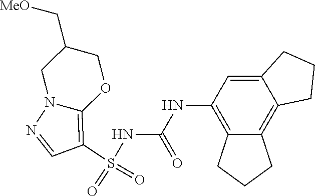 [SMILES: COCC1COc2c(S(=O)(=O)NC(=O)Nc3cc4c(c5c3CCC5)CCC4)cnn2C1] |
| 189 | 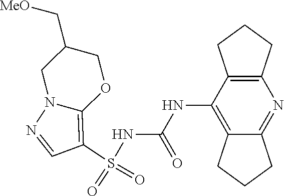 [SMILES: COCC1COc2c(S(=O)(=O)NC(=O)Nc3c4c(nc5c3CCC5)CCC4)cnn2C1] |
| 190 | 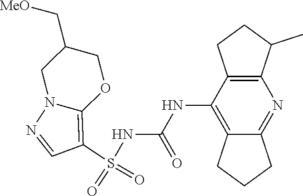 [SMILES: COCC1COc2c(S(=O)(=O)NC(=O)Nc3c4c(nc5c3CCC5C)CCC4)cnn2C1] |
| 191 | 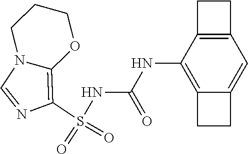 [SMILES: O=C(Nc1c2c(cc3c1CC3)CC2)NS(=O)(=O)c1ncn2c1OCCC2] |
| 192 | 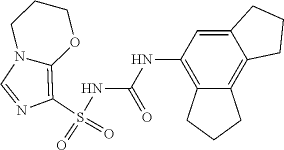 [SMILES: O=C(Nc1cc2c(c3c1CCC3)CCC2)NS(=O)(=O)c1ncn2c1OCCC2] |
| 193 | 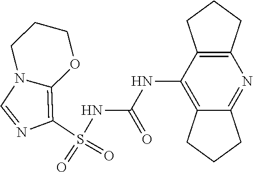 [SMILES: O=C(Nc1c2c(nc3c1CCC3)CCC2)NS(=O)(=O)c1ncn2c1OCCC2] |
| 194 | 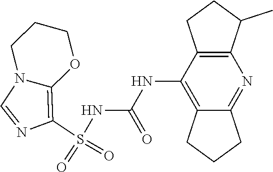 [SMILES: CC1CCc2c1nc1c(c2NC(=O)NS(=O)(=O)c2ncn3c2OCCC3)CCC1] |
| 195 | 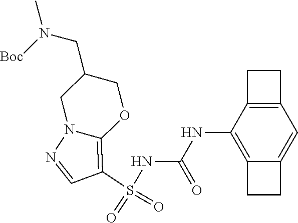 [SMILES: CN(CC1COc2c(S(=O)(=O)NC(=O)Nc3c4c(cc5c3CC5)CC4)cnn2C1)C(=O)OC(C)(C)C] |
| 196 | 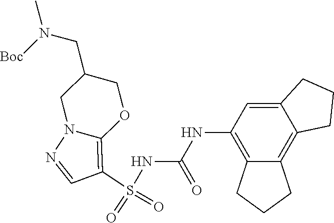 [SMILES: CN(CC1COc2c(S(=O)(=O)NC(=O)Nc3cc4c(c5c3CCC5)CCC4)cnn2C1)C(=O)OC(C)(C)C] |
| 197 | 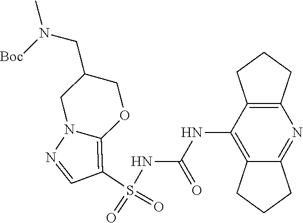 [SMILES: CN(CC1COc2c(S(=O)(=O)NC(=O)Nc3c4c(nc5c3CCC5)CCC4)cnn2C1)C(=O)OC(C)(C)C] |
| 198 | 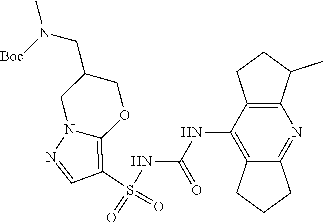 [SMILES: CC1CCc2c1nc1c(c2NC(=O)NS(=O)(=O)c2cnn3c2OCC(CN(C)C(=O)OC(C)(C)C)C3)CCC1] |
| 199 | 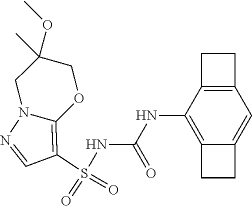 [SMILES: COC1(C)COc2c(S(=O)(=O)NC(=O)Nc3c4c(cc5c3CC5)CC4)cnn2C1] |
| 200 | 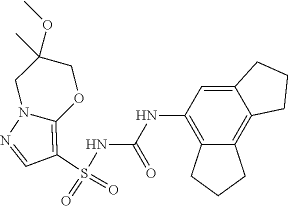 [SMILES: COC1(C)COc2c(S(=O)(=O)NC(=O)Nc3cc4c(c5c3CCC5)CCC4)cnn2C1] |
| 201 | 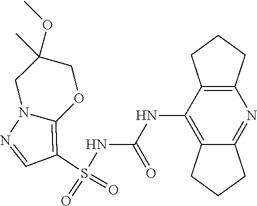 [SMILES: COC1(C)COc2c(S(=O)(=O)NC(=O)Nc3c4c(nc5c3CCC5)CCC4)cnn2C1] |
| 202 | 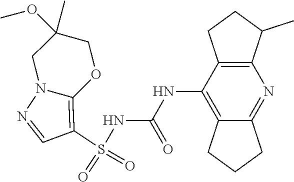 |
| 203 | 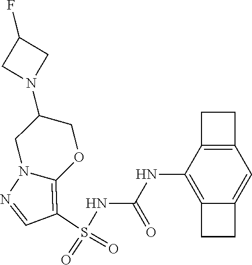 [SMILES: O=C(Nc1c2c(cc3c1CC3)CC2)NS(=O)(=O)c1cnn2c1OCC(N1CC(F)C1)C2] |
| 204 | 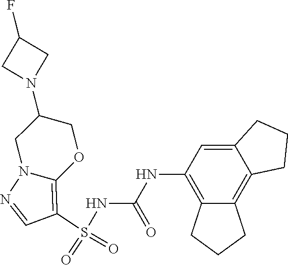 [SMILES: O=C(Nc1cc2c(c3c1CCC3)CCC2)NS(=O)(=O)c1cnn2c1OCC(N1CC(F)C1)C2] |
| 205 | 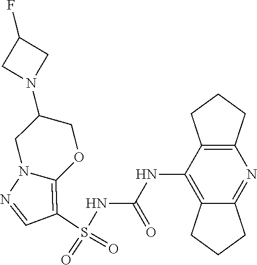 [SMILES: O=C(Nc1c2c(nc3c1CCC3)CCC2)NS(=O)(=O)c1cnn2c1OCC(N1CC(F)C1)C2] |
| 206 | 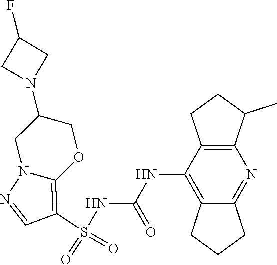 [SMILES: CC1CCc2c1nc1c(c2NC(=O)NS(=O)(=O)c2cnn3c2OCC(N2CC(F)C2)C3)CCC1] |
| 207 | 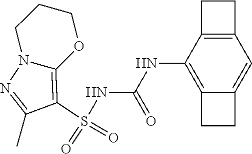 [SMILES: Cc1nn2c(c1S(=O)(=O)NC(=O)Nc1c3c(cc4c1CC4)CC3)OCCC2] |
| 208 | 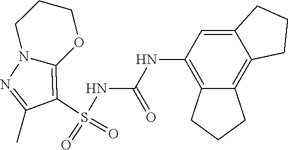 [SMILES: Cc1nn2c(c1S(=O)(=O)NC(=O)Nc1cc3c(c4c1CCC4)CCC3)OCCC2] |
| 209 | 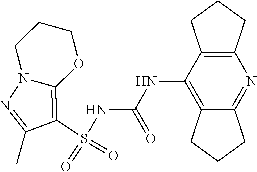 [SMILES: Cc1nn2c(c1S(=O)(=O)NC(=O)Nc1c3c(nc4c1CCC4)CCC3)OCCC2] |
| 210 | 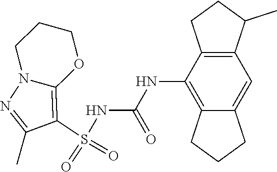 [SMILES: Cc1nn2c(c1S(=O)(=O)NC(=O)Nc1c3c(cc4c1CCC4C)CCC3)OCCC2] |
| 211 | 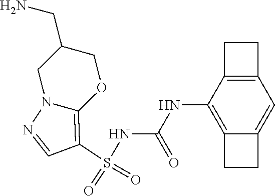 [SMILES: NCC1COc2c(S(=O)(=O)NC(=O)Nc3c4c(cc5c3CC5)CC4)cnn2C1] |
| 212 | 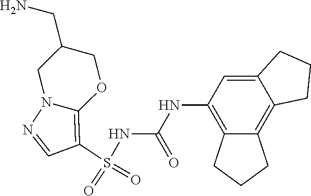 [SMILES: NCC1COc2c(S(=O)(=O)NC(=O)Nc3cc4c(c5c3CCC5)CCC4)cnn2C1] |
| 213 | 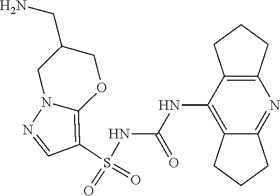 [SMILES: NCC1COc2c(S(=O)(=O)NC(=O)Nc3c4c(nc5c3CCC5)CCC4)cnn2C1] |
| 214 | 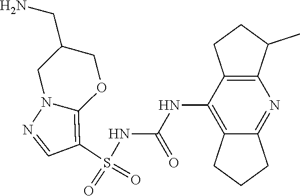 [SMILES: CC1CCc2c1nc1c(c2NC(=O)NS(=O)(=O)c2cnn3c2OCC(CN)C3)CCC1] |
| 215 | 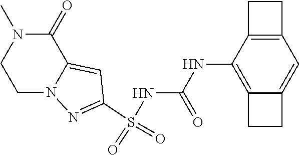 [SMILES: CN1CCn2nc(S(=O)(=O)NC(=O)Nc3c4c(cc5c3CC5)CC4)cc2C1=O] |
| 216 | 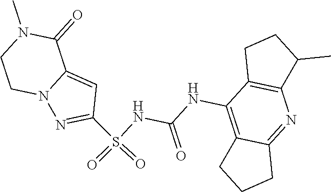 [SMILES: CC1CCc2c1nc1c(c2NC(=O)NS(=O)(=O)c2cc3n(n2)CCN(C)C3=O)CCC1] |
| 217 | 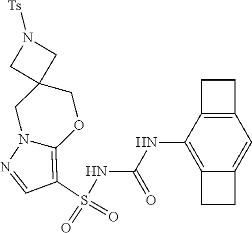 [SMILES: Cc1ccc(S(=O)(=O)N2CC3(COc4c(S(=O)(=O)NC(=O)Nc5c6c(cc7c5CC7)CC6)cnn4C3)C2)cc1] |
| 218 | 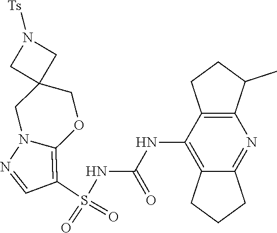 [SMILES: Cc1ccc(S(=O)(=O)N2CC3(COc4c(S(=O)(=O)NC(=O)Nc5c6c(nc7c5CCC7C)CCC6)cnn4C3)C2)cc1] |
| 219 | 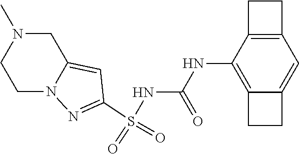 [SMILES: CN1CCn2nc(S(=O)(=O)NC(=O)Nc3c4c(cc5c3CC5)CC4)cc2C1] |
| 220 | 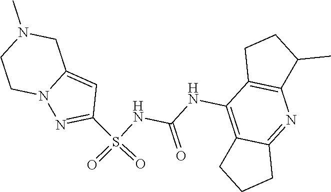 [SMILES: CC1CCc2c1nc1c(c2NC(=O)NS(=O)(=O)c2cc3n(n2)CCN(C)C3)CCC1] |
| 221 | 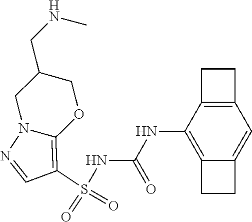 [SMILES: CNCC1COc2c(S(=O)(=O)NC(=O)Nc3c4c(cc5c3CC5)CC4)cnn2C1] |
| 222 | 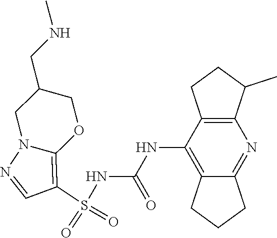 [SMILES: CNCC1COc2c(S(=O)(=O)NC(=O)Nc3c4c(nc5c3CCC5C)CCC4)cnn2C1] |
| 223 | 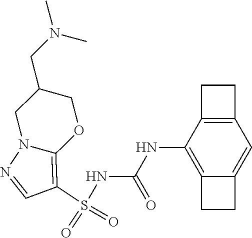 [SMILES: CN(C)CC1COc2c(S(=O)(=O)NC(=O)Nc3c4c(cc5c3CC5)CC4)cnn2C1] |
| 224 | 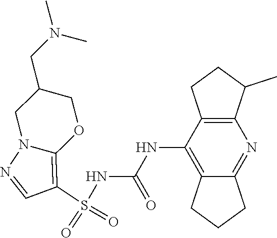 [SMILES: CC1CCc2c1nc1c(c2NC(=O)NS(=O)(=O)c2cnn3c2OCC(CN(C)C)C3)CCC1] |
| 225 | 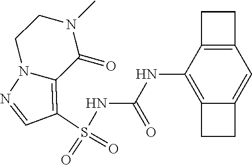 [SMILES: CN1CCn2ncc(S(=O)(=O)NC(=O)Nc3c4c(cc5c3CC5)CC4)c2C1=O] |
| 226 | 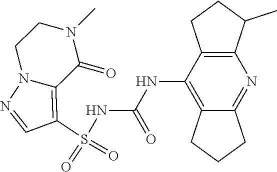 [SMILES: CC1CCc2c1nc1c(c2NC(=O)NS(=O)(=O)c2cnn3c2C(=O)N(C)CC3)CCC1] |
| 227 | 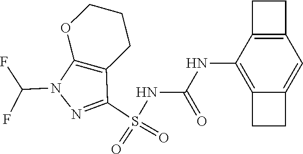 [SMILES: O=C(Nc1c2c(cc3c1CC3)CC2)NS(=O)(=O)c1nn(C(F)F)c2c1CCCO2] |
| 228 | 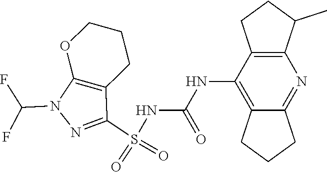 [SMILES: CC1CCc2c1nc1c(c2NC(=O)NS(=O)(=O)c2nn(C(F)F)c3c2CCCO3)CCC1] |
| 229 | 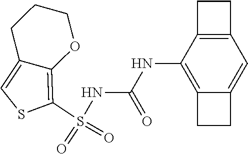 [SMILES: O=C(Nc1c2c(cc3c1CC3)CC2)NS(=O)(=O)c1scc2c1OCCC2] |
| 230 | 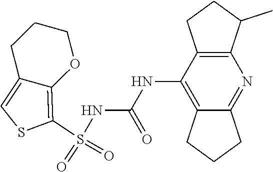 [SMILES: CC1CCc2c1nc1c(c2NC(=O)NS(=O)(=O)c2scc3c2OCCC3)CCC1] |
| 231 | 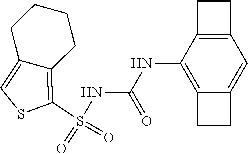 [SMILES: O=C(Nc1c2c(cc3c1CC3)CC2)NS(=O)(=O)c1scc2c1CCCC2] |
| 232 | 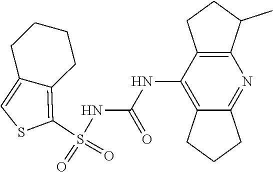 [SMILES: CC1CCc2c1nc1c(c2NC(=O)NS(=O)(=O)c2scc3c2CCCC3)CCC1] |
| 233 | 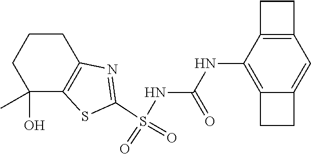 [SMILES: CC1(O)CCCc2nc(S(=O)(=O)NC(=O)Nc3c4c(cc5c3CC5)CC4)sc21] |
| 234 | 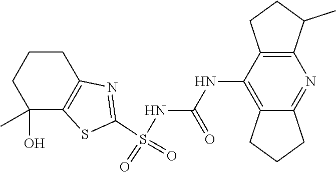 |
| 235 | 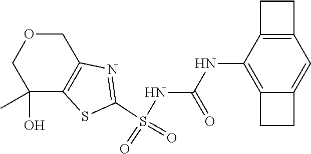 [SMILES: CC1(O)COCc2nc(S(=O)(=O)NC(=O)Nc3c4c(cc5c3CC5)CC4)sc21] |
| 236 | 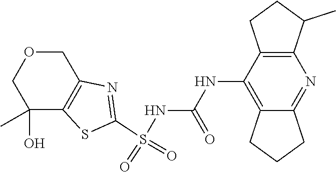 |

and pharmaceutically acceptable salts thereof.

In some embodiments, provided herein is a compound that is selected from the group consisting of the compounds in Table 1B:

TABLE 1B

| Comp. | |
|---|---|
| No. | Structure |
| 8 | 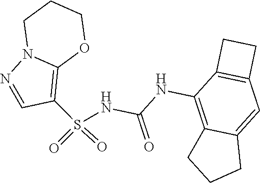 [SMILES: O=C(Nc1c2c(cc3c1CC3)CCC2)NS(=O)(=O)c1cnn2c1OCCC2] |
| 9 | 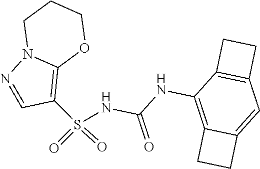 [SMILES: O=C(Nc1c2c(cc3c1CC3)CC2)NS(=O)(=O)c1cnn2c1OCCC2] |
| 10 | 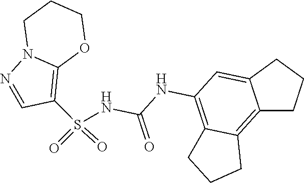 [SMILES: O=C(Nc1cc2c(c3c1CCC3)CCC2)NS(=O)(=O)c1cnn2c1OCCC2] |
| 12 | 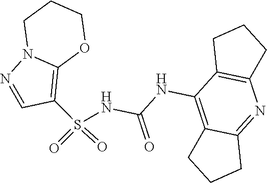 [SMILES: O=C(Nc1c2c(nc3c1CCC3)CCC2)NS(=O)(=O)c1cnn2c1OCCC2] |
| 13 | 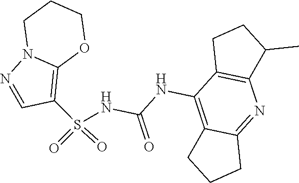 [SMILES: CC1CCc2c1nc1c(c2NC(=O)NS(=O)(=O)c2cnn3c2OCCC3)CCC1] |
| 14 | 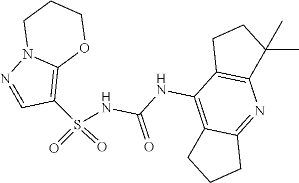 [SMILES: CC1(C)CCc2c1nc1c(c2NC(=O)NS(=O)(=O)c2cnn3c2OCCC3)CCC1] |
| 15 | 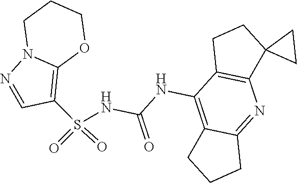 [SMILES: O=C(Nc1c2c(nc3c1CCC31CC1)CCC2)NS(=O)(=O)c1cnn2c1OCCC2] |
| 16 | 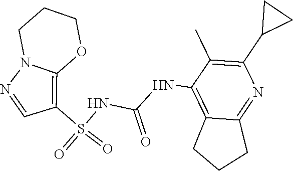 [SMILES: Cc1c(C2CC2)nc2c(c1NC(=O)NS(=O)(=O)c1cnn3c1OCCC3)CCC2] |

and pharmaceutically acceptable salts thereof.

In another aspect, provided herein is a compound having the structure:

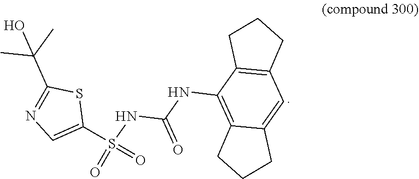 [SMILES: CC(C)(O)c1ncc(S(=O)(=O)NC(=O)Nc2c3c(cc4c2CCC4)CCC3)s1]

In some embodiments, provided herein is a compound that is selected from the group consisting of the compounds in Table 1C:

| Comp. | |
|---|---|
| No. | Structure |
| 401 | 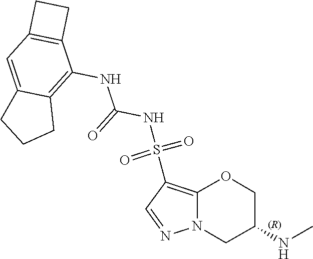 |
| 402 | 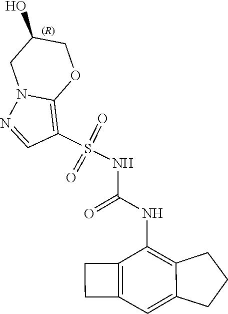 |
| 403 | 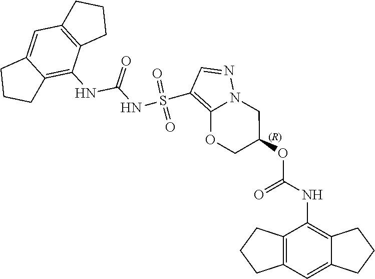 |
| 404 | 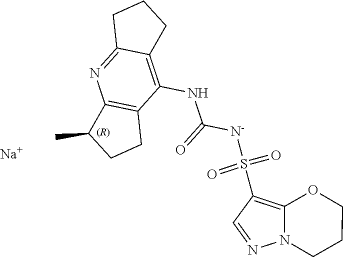 |

In another aspect, provided herein is a compound of Formula AB,

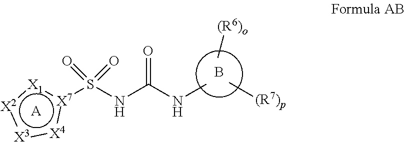

wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, or NR⁴;

### X⁷ is N or C;

each of R¹, R², R³, and R⁴, when present, is each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;

R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;

R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;

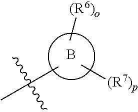

is selected from the group consisting of:

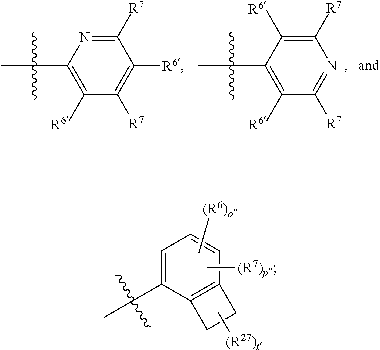

o″=0 or 1;
p″=0, 1, or 2;
t′ is 0, 1, 2, 3, or 4;
R⁶ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R⁷ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷;
R^{6′} at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
or at least one pair of R^{6′} and R⁷ on adjacent atoms, taken together with the atoms connecting them, independently form at least one C₄-C₈ cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
R²⁷, at each occurrence, is independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl; or a pharmaceutically acceptable salt thereof.

In another aspect, provided herein is a compound of Formula AB,

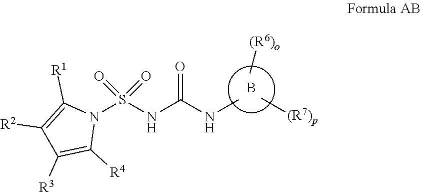

wherein
each of R¹, R², R³, and R⁴, when present, is each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;

R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;

R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;

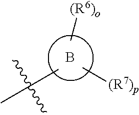

is selected from the group consisting of:

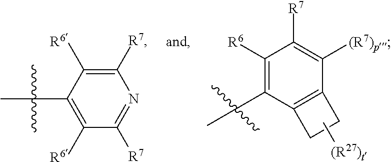

p′″=0 or 1;
t′ is 0, 1, 2, 3, or 4;
R⁶ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R⁷ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷;
both pairs of R^{6′} and R⁷ on adjacent atoms, taken together with the atoms connecting them, independently form at least one C₄-C₈ cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
R²⁷, at each occurrence, is independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
or a pharmaceutically acceptable salt thereof.

In some embodiments of the compound of Formula AB,

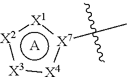

is selected from

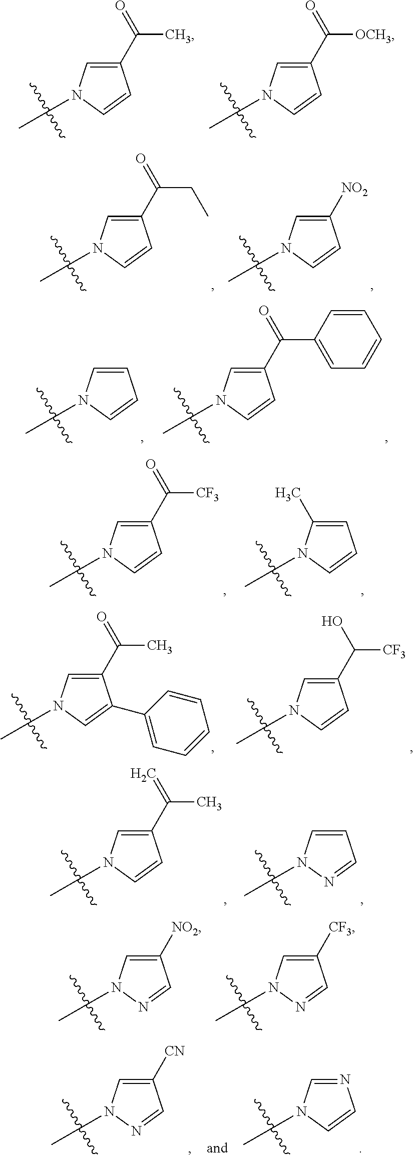

In some embodiments of the compound of Formula AB,

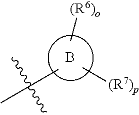

is selected from the group consisting of:

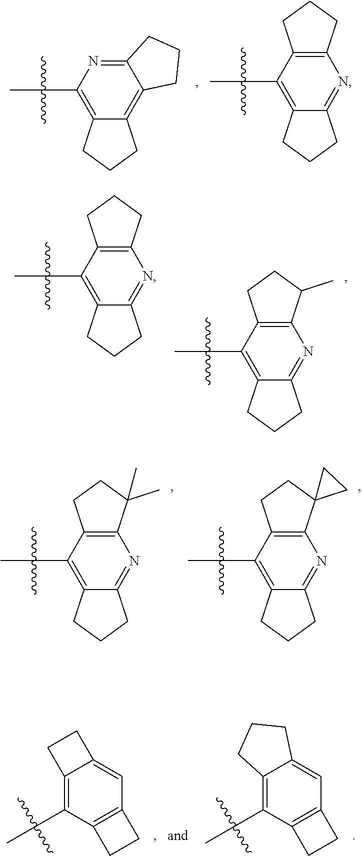

In some embodiments of the compound of Formula AB,

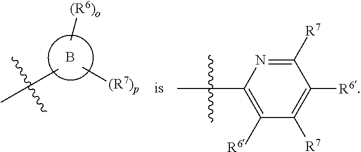

In certain embodiments,

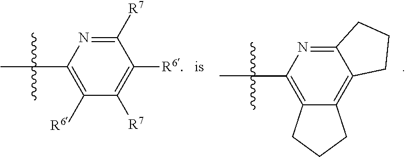

In some embodiments of the compound of Formula AB,

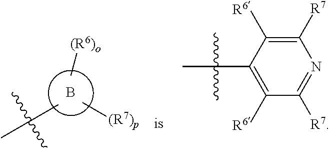

In certain embodiments,

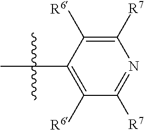

is selected from

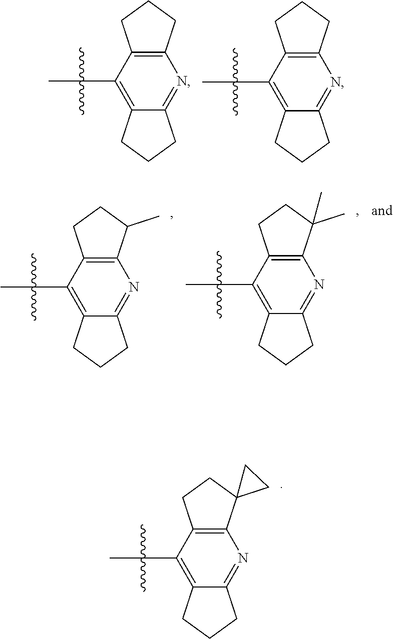

In some embodiments of the compound of Formula AB,

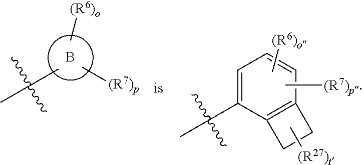

In certain embodiments,

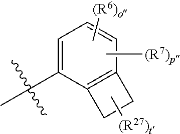

is selected from

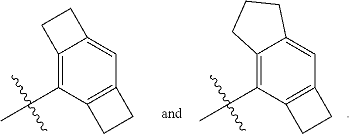 [SMILES: *c1c2c(cc3c1CC3)CC2 ; *c1c2c(cc3c1CC3)CCC2]

In some embodiments of the compound of Formula AB,

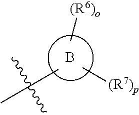

is selected

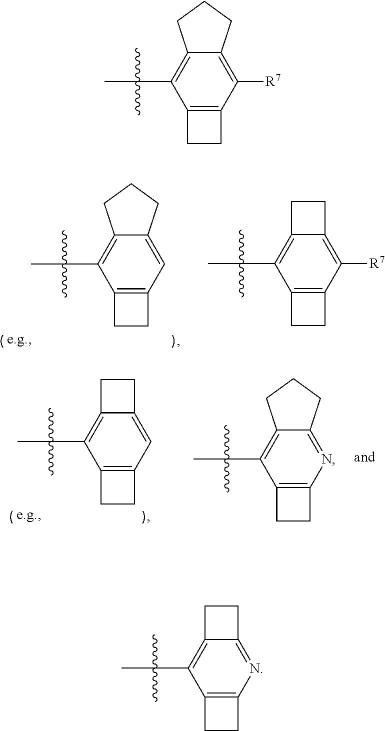

from

In some embodiments of the compound of Formula AB,

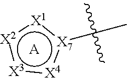

is selected from

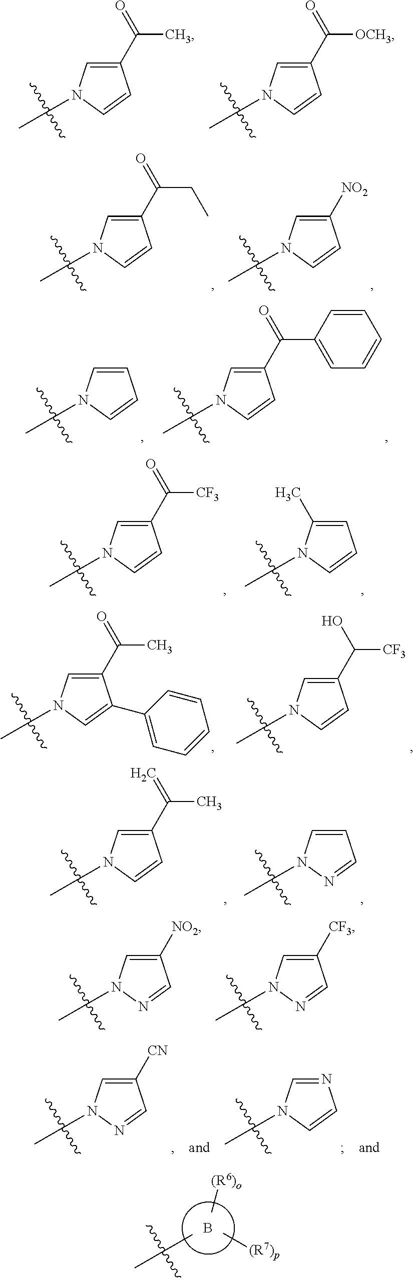

is selected from

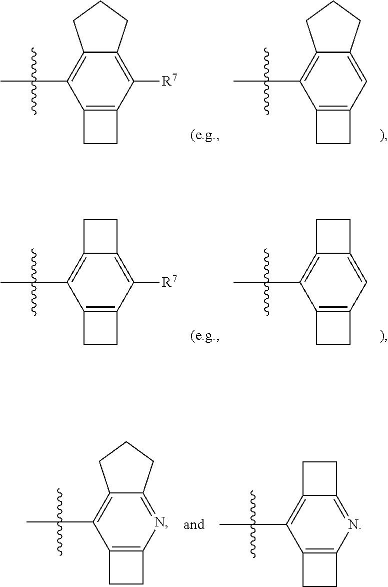

In some embodiments of the compound of Formula AB, the compound is selected from the group consisting of:

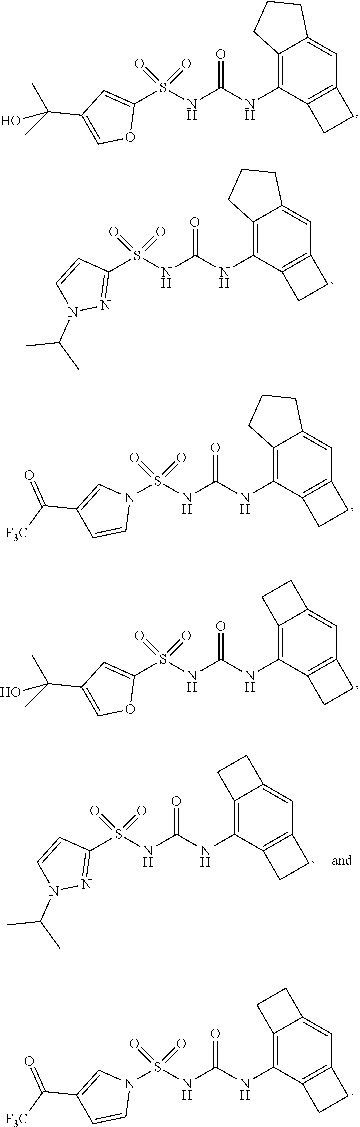 [SMILES: CC(C)(O)c1coc(S(=O)(=O)NC(=O)Nc2c3c(cc4c2CC4)CCC3)c1 ; CC(C)n1ccc(S(=O)(=O)NC(=O)Nc2c3c(cc4c2CC4)CCC3)n1 ; O=C(Nc1c2c(cc3c1CC3)CCC2)NS(=O)(=O)n1ccc(C(=O)C(F)(F)F)c1 ; CC(C)(O)c1coc(S(=O)(=O)NC(=O)Nc2c3c(cc4c2CC4)CC3)c1 ; CC(C)n1ccc(S(=O)(=O)NC(=O)Nc2c3c(cc4c2CC4)CC3)n1 ; O=C(Nc1c2c(cc3c1CC3)CC2)NS(=O)(=O)n1ccc(C(=O)C(F)(F)F)c1]

In some embodiments of the compound of Formula AB, the compound is selected from the group consisting of:

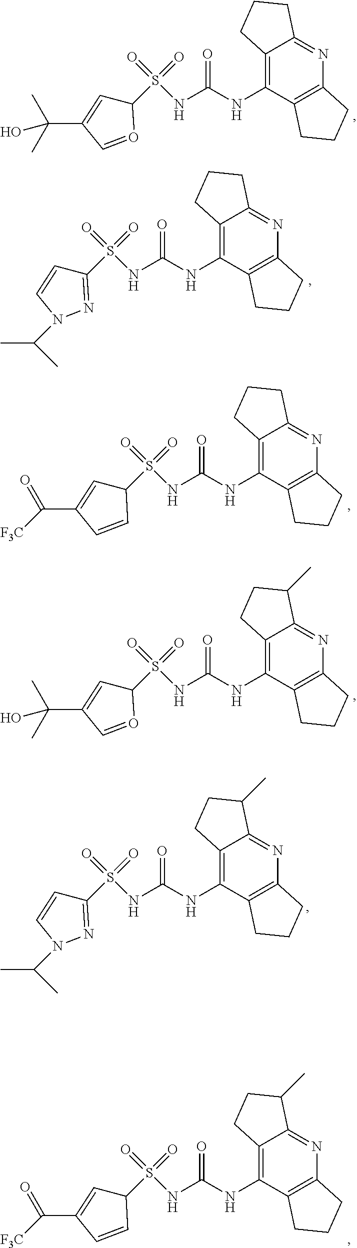

In some embodiments of the compound of Formula AB, the compound is selected from the group consisting of:

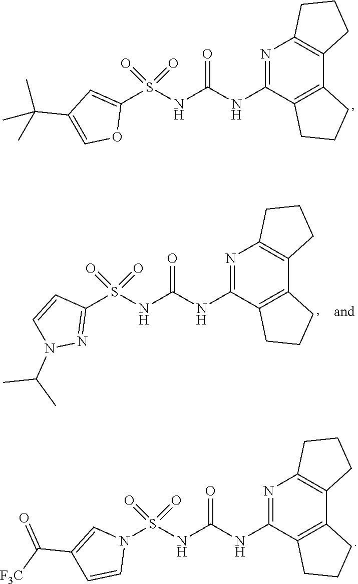 [SMILES: CC(C)(C)c1coc(S(=O)(=O)NC(=O)Nc2nc3c(c4c2CCC4)CCC3)c1 ; CC(C)n1ccc(S(=O)(=O)NC(=O)Nc2nc3c(c4c2CCC4)CCC3)n1 ; O=C(Nc1nc2c(c3c1CCC3)CCC2)NS(=O)(=O)n1ccc(C(=O)C(F)(F)F)c1]

In some embodiments of the compound of Formula AB, the compound is selected from the group consisting of:

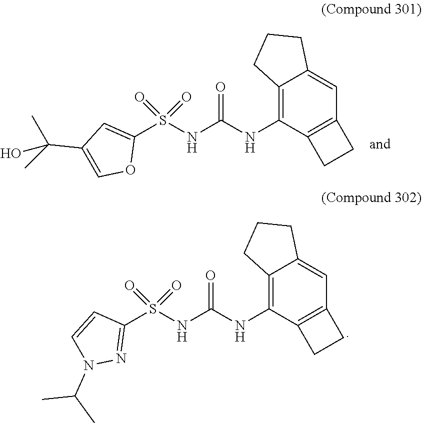 [SMILES: CC(C)(O)c1coc(S(=O)(=O)NC(=O)Nc2c3c(cc4c2CC4)CCC3)c1 ; CC(C)n1ccc(S(=O)(=O)NC(=O)Nc2c3c(cc4c2CC4)CCC3)n1]

In some embodiments, a compound provided herein is

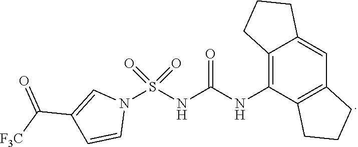 [SMILES: O=C(Nc1c2c(cc3c1CCC3)CCC2)NS(=O)(=O)n1ccc(C(=O)C(F)(F)F)c1]

Pharmaceutical Compositions and Administration

General

In some embodiments, a chemical entity (e.g., a compound that modulates (e.g., antagonizes) NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, an NLRP3 antagonist and/or an anti-TNFα agent disclosed herein is administered as a pharmaceutical composition that includes the NLRP3 antagonist and/or anti-TNFα agent and one or more pharmaceutically acceptable excipients, and optionally one or more additional therapeutic agents as described herein. Preferably the pharmaceutical composition that includes an NLRP3 antagonist and an anti-TNFα agent.

Preferably the above pharmaceutical composition embodiments comprise an NLRP3 antagonist disclosed herein. More preferably the above pharmaceutical composition embodiments comprise an NLRP3 antagonist and an anti-TNFα agent disclosed herein.

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C₁-C₄ alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dxtrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phospahate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula AA, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the chemical entity is a compound of Formula AA, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., a compound of Formula AA, or a pharmaceutically acceptable salt and/or hydrate and/or cocrystal thereof; e.g., a compound of Formula AA) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula AA, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound of | 40 weight percent to about 80 weight |
| Formula AA | percent (e.g., from about 50 weight |
| | percent to about 70 weight percent, |
| | from about 55 weight percent to about |
| | 70 weight percent; from about 60 |
| | weight percent to about 65 weight |
| | percent; e.g., about 62.1 weight percent) |
| Crospovidone | 0.5 weight percent to about 5 weight |
| (Kollidon CL) | percent (e.g., from about 0.5 weight |
| | percent to about 3 weight percent, from |
| | about 1 weight percent to about 3 weight |
| | percent; about 1.93 weight percent |
| lactose | about 10 weight percent to about 50 |
| monohydrate | weight percent (e.g., from about 20 weight |
| (Pharmatose | percent to about 40 weight percent, from |
| 200M) | about 25 weight percent to about 35 weight |
| | percent; e.g., about 31.03 weight percent |
| Povidone | about 0.5 weight percent to about 5 weight |
| (Kollidon | percent (e.g., from about 1.5 weight percent |
| K30) | to about 4.5 weight percent, from about 2 |
| | weight percent to about 3.5 weight percent; |
| | e.g., about 2.76 weight percent |
| talc | 0.5 weight percent to about 5 weight percent |
| | (e.g., from about 0.5 weight percent to about 3 |
| | weight percent, from about 1 weight percent to |
| | about 3 weight percent; from about 1.5 weight |
| | percent to about 2.5 weight percent; from about |
| | 1.8 weight percent to about 2.2 weight percent; |
| | e.g., about 1.93 weight percent |
| Magnesium | about 0.05 weight percent to about 1 weight |
| stearate | percent (e.g., from about 0.05 weight percent to |
| | about 1 weight percent; from about 0.1 weight |
| | percent to about 1 weight percent; from about |
| | 0.1 weight percent to about 0.5 weight percent; |
| | e.g., about 0.27 weight percent |

In certain embodiments, component (i includes the ingredients and amounts as shown in Table B.

TABLE B

| Ingredient | Weight Percent |
|---|---|
| A compound of Formula AA | About 62.1 weight percent) |
| Crospovidone (Kollidon CL) | About 1.93 weight percent |
| lactose monohydrate | About 31.03 weight percent |
| (Pharmatose 200M) | |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phospahate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a″′) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c″′) a second buffer, such as phosphate buffer system (e.g., sodium dihydrogen phospahate dehydrate), In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a″′).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b″′).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c″′).

In certain of these embodiments, each of (a″)-(c″′) is present.

In certain embodiments, component (ii) includes water (up to 100%) and the ingredients and amounts as shown in Table C.

TABLE C

| Ingredient | Weight Percent |
|---|---|
| methyl cellulose | 0.05 weight percent to about 5 weight |
| (Methocel A15C | percent (e.g., from about 0.05 weight |
| premium) | percent to about 3 weight percent, from |
| | about 0.1 weight percent to about 3 |
| | weight percent; e.g., about 1.4 weight |
| | percent |
| Povidone | 0.05 weight percent to about 5 weight |
| (Kollidon K30) | percent (e.g., from about 0.05 weight |
| | percent to about 3 weight percent, from |
| | about 0.1 weight percent to about 2 |
| | weight percent; e.g., about 1.0 weight |
| | percent |
| propyl 4- | about 0.005 weight percent to about |
| hydroxybenzoate | 0.1 weight percent (e.g., from about 0.005 |
| | weight percent to about 0.05 weight |
| | percent; e.g., about 0.02 weight percent) |
| methyl 4- | about 0.05 weight percent to about 1 |
| hydroxybenzoate | weight percent (e.g., from about 0.05 |
| | weight percent to about 0.5 weight |
| | percent; e.g., about 0.20 weight percent) |
| disodium | about 0.05 weight percent to about 1 |
| phosphate | weight percent (e.g., from about 0.05 |
| dodecahydrate | weight percent to about 0.5 weight |
| | percent; e.g., about 0.15 weight percent) |
| sodium dihydrogen | about 0.005 weight percent to about 0.5 |
| phospahate | weight percent (e.g., from about 0.005 |
| dihydrate | weight percent to about 0.3 weight |
| | percent; e.g., about 0.15 weight percent) |

In certain embodiments, component (ii) includes water (up to 1000%) and the ingredients and amounts as shown in Table D.

TABLE D

| Ingredient | Weight Percent |
|---|---|
| methyl cellulose (Methocel | about 1.4 weight percent |
| A15C premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate | about 0.15 weight percent |
| dodecahydrate | |
| sodium dihydrogen phospahate | about 0.15 weight percent |
| dihydrate | |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

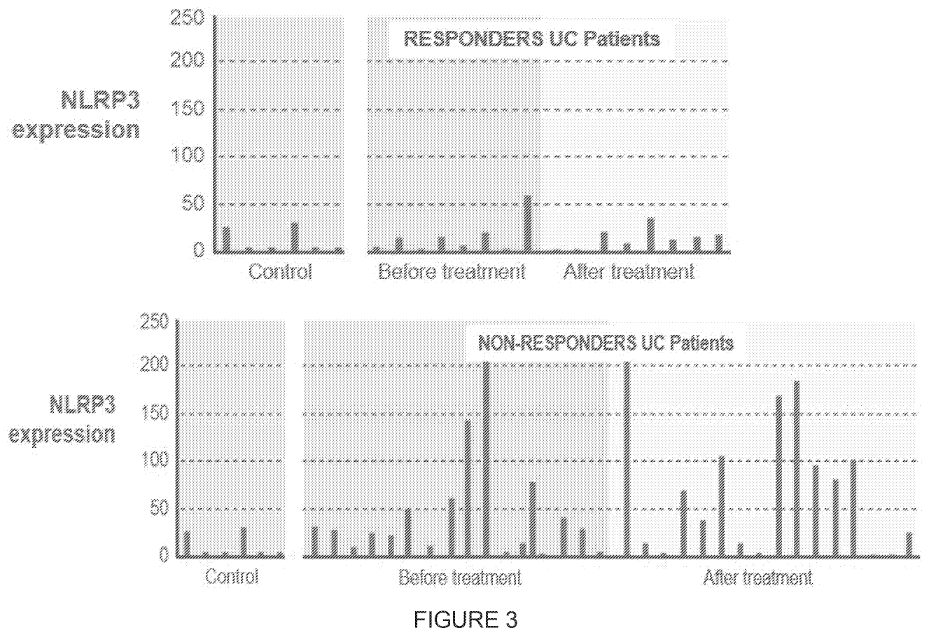

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device shown in FIGS. 3A-3C, which includes a plastic bottle, a breakable capsule, and a rectal cannula and single flow pack.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 150 mg of a compound of Formula AA in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 450 mg of a compound of Formula AA in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is a compound of Formula AA, or a pharmaceutically acceptable salt and/or hydrate and/or cocrystal thereof. For example, enema formulations can include about 2.5 mg/mL or about 7.5 mg/mL of a compound of Formula AA in liquid carrier.

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from major adverse cardiovascular events such as carbiovascular death, non-fatal myocardial infarction and non-fatal stroke in patients with a prior hear attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity.

In some embodiments, the condition, disease or disorder is Type 2 Diabetes.

In some embodiments, the condition, disease or disorder is NASH.

In some embodiments, the condition, disease or disorder is Alzheimer's disease.

In some embodiments, the condition, disease or disorder is gout.

In some embodiments, the condition, disease or disorder is SLE.

In some embodiments, the condition, disease or disorder is rheumatoid arthritis.

In some embodiments, the condition, disease or disorder is IBD.

In some embodiments, the condition, disease or disorder is multiple sclerosis.

In some embodiments, the condition, disease or disorder is COPD.

In some embodiments, the condition, disease or disorder is asthma.

In some embodiments, the condition, disease or disorder is scleroderma.

In some embodiments, the condition, disease or disorder is pulmonary fibrosis.

In some embodiments, the condition, disease or disorder is age related macular degeneration (AMD).

In some embodiments, the condition, disease or disorder is cystic fibrosis.

In some embodiments, the condition, disease or disorder is Muckle Wells syndrome.

In some embodiments, the condition, disease or disorder is familial cold autoinflammatory syndrome (FCAS).

In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis.

In some embodiments, the indication is MDS.

In some embodiments, the indication is non-small lung cancer in patients carrying mutation or overexpression of NLRP3.

In some embodiments, the indication is ALL in patients resistant to glucocorticoids treatment.

In some embodiments, the indication is LCH.

In some embodiments, the indication is multiple myeloma.

In some embodiments, the indication is promyelocytic leukemia.

In some embodiments, the indication is gastric cancer.

In some embodiments, the indication is lung cancer metastasis.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20.

In some embodiments, the methods described herein further include the step of identifying a subject (e.g., a patient) in need of treatment for an indication related to NLRP3 activity, such as an indication related to point mutation of NLRP3 signaling.

Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibitreduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (INK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., acell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), INK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP).

For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, INK, LBP, MEK1/2, MEK3/6, MEK4/7, MK2, MyD88, NF-xB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, INK, lipopolysaccharide binding protein (LBP), MEK1/2, MEK3/6, MEK4/7, MK2, MyD88, NF-κB, NIK, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH₃, a Diabody-CH₃, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kλ-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)-IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian J. Gastroenterol. Hepatol. 15(6):376-384, 2001; Elliott et al., Lancet 1994; 344: 1125-1127, 1994; Feldmann et al., Ann. Rev. Immunol. 19(1):163-196, 2001; Rankin et al., Br. J. Rheumatol. 2:334-342, 1995; Knight et al., Molecular Immunol. 30(16):1443-1453, 1993; Lorenz et al., J. Immunol. 156(4):1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12):1947-1955, 2013; Palladino et al., Nature Rev. Drug Discovery 2(9):736-746, 2003; Sandborn et al., Inflammatory Bowel Diseases 5(2):119-133, 1999; Atzeni et al., Autoimmunity Reviews 12(7):703-708, 2013; Maini et al., Immunol. Rev. 144(1):195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (Golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GS071 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or IYL-1401-A from Biocon/Mylan, CHS-1420 from Coherus, FKB327 from Kyowa Kirin, and BI 695501 from Boehringer Ingelheim; Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵ M (e.g., less than 0.5×10⁻⁵ M, less than 1×10⁻⁶ M, less than 0.5×10⁻⁶ M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻⁹ M, less than 1×10⁻¹⁰ M, less than 0.5×10⁻¹⁰ M, less than 1×10⁻¹ M, less than 0.5×10⁻¹ M, or less than 1×10⁻¹² M), e.g., as measured in phosphate buffered saline using surface plasmon resonance (SPR).

In some embodiments, any of the antibodies or antigen-binding fragments described herein has a K_{D} of about 1×10⁻¹² M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹ M, or about 0.5×10⁻¹ M (inclusive); about 0.5×10⁻¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, or about 1×10⁻¹¹ M (inclusive); about 1×10⁻⁴ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, or about 0.5×10⁻¹⁰ M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, or about 1×10⁻¹⁰ M (inclusive); about 1×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, or about 1×10⁻⁹ M (inclusive); about 1×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, or about 0.5×10⁻⁸M (inclusive); about 0.5×10⁻⁸ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, or about 1×10⁻⁸ M (inclusive); about 1×10⁻⁸ M to about 1×10⁻⁵ M, about 0.5×10⁻⁵ M, about 1×10⁻⁶ M, about 0.5×10⁻⁶ M, about 1×10⁻⁷ M, or about 0.5×10⁻⁷ M (inclusive); about 0.5×10⁻⁷ M to about 1×10⁻⁵ M, about 0.5×10⁻⁵ M, about 1×10⁻⁶ M, about 0.5×10⁻⁶ M, or about 1×10⁻⁷ M (inclusive); about 1×10⁻⁷ M to about 1×10⁻⁵ M, about 0.5×10⁻⁵ M, about 1×10⁻⁶ M, or about 0.5×10⁻⁶ M (inclusive); about 0.5×10⁻⁶ M to about 1×10⁻⁵ M, about 0.5×10⁻⁵ M, or about 1×10⁻⁶ M (inclusive); about 1×10⁻⁶ M to about 1×10⁻⁵ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR).

In some embodiments, any of the antibodies or antigen-binding fragments described herein has a Koff of about 1×10⁻⁶ s⁻¹ to about 1×10⁻³ s⁻¹, about 0.5×10⁻³ s⁻¹, about 1×10⁻⁴ s⁻¹, about 0.5×10⁻⁴ s⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹, about 1×10⁻⁴ s⁻¹, about 0.5×10⁻⁴ s⁻¹, or about 1×10⁻⁵ s⁻¹ (inclusive); about 1×10⁻⁵ s⁻¹ to about 1×10⁻³ s⁻¹, about 0.5×10⁻³ s⁻¹, about 1×10⁻⁴ s⁻¹, or about 0.5×10⁻⁴ s⁻¹ (inclusive); about 0.5×10⁻⁴ s⁻¹ to about 1×10⁻³ s⁻¹, about 0.5×10⁻³ s⁻¹, or about 1×10⁻⁴ s⁻¹ (inclusive); about 1×10⁻⁴ s⁻¹ to about 1×10⁻³ s⁻¹, or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10² M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, about 0.5×10⁴ M⁻¹s⁻¹, about 1×10³ M⁻¹s⁻¹, or about 0.5×10³ M⁻¹s⁻¹ (inclusive); about 0.5×10³ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, about 0.5×10⁴ M⁻¹s⁻¹, or about 1×10³ M⁻¹s⁻¹ (inclusive); about 1×10³ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, or about 0.5×10⁴ M⁻¹s⁻¹ (inclusive); about 0.5×10⁴ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, or about 1×10⁴ M⁻¹s⁻¹ (inclusive); about 1×10⁴ M⁻¹s⁻¹ to about 1×10⁶ M⁻¹s⁻¹, about 0.5×10⁶ M⁻¹s⁻¹, about 1×10⁵ M⁻¹s⁻¹, or about 0.5×10⁵ M⁻¹s⁻¹ (inclusive); about 0.5×10⁵ M⁻¹s⁻¹ to about 1×10⁶ M⁻¹s⁻¹, about 0.5×10⁶ M⁻¹s⁻¹, or about 1×10⁵ M⁻¹s⁻¹ (inclusive); about 1×10⁵ M⁻¹s⁻¹ to about 1×10⁶ M⁻¹s⁻¹, or about 0.5×10⁶ M⁻¹s⁻¹ (inclusive); or about 0.5×10⁶ M⁻¹s⁻¹ to about 1×10⁶M⁻¹s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. J. Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013, 1999; Mohler et al., J. Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J. Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is fully or partially complementary to all or part of a AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., fully or partially complementary to all or a part of any one of the sequences presented in Table E).

TABLE E

| | mRNA GenBank |
|---|---|
| Human gene | accession number(s) |
| Tumor necrosis factor (TNF, a.k.a. TNF- | NM_000594 |
| alpha) | |
| TNF receptor superfamily member 1A | NM_001065 |
| (TNFRSF1A) (a.k.a. TNFR1) | NM_001346091 |
| | NM_001346092 |
| TNF receptor superfamily member 1B | NM_001066 |
| (TNFRSF1B) (a.k.a. TNFR2) | XM_011542060 |
| | XM_011542063 |
| | XM_017002214 |
| | XM_017002215 |
| | XM_017002211 |
| TNFRSF1A associated via death domain | NM_003789 |
| (TRADD) | NM_001323552 |
| | XM_005256213 |
| | XM_017023815 |
| TNF receptor associated factor 2 (TRAF2) | NM_021138 |
| | XM_011518976 |
| | XM_011518977 |
| | XM_011518974 |
| JunD proto-oncogene, AP-1 transcription | NM_001286968 |
| factor subunit (JUND) | NM_005354 |
| Mitogen-activated protein kinase kinase | NM_005923 |
| kinase 5 (MAP3K5) (a.k.a. ASK1) | XM_017010875 |
| | XM_017010872 |
| | XM_017010873 |
| | XM_017010877 |
| | XM_017010874 |
| | XM_017010871 |
| | XM_017010870 |
| | XM_017010876 |
| | XM_011535839 |
| CD14 | NM_000591 |
| | NM_001040021 |
| | NM_001174104 |
| | NM_001174105 |
| Mitogen-activated protein kinase 3 | NM_001040056 |
| (MAPK3) (a.k.a. ERK1) | NM_001109891 |
| | NM_002746 |
| Mitogen-activated protein kinase 1 | NM_002745 |
| (MAPK1) (a.k.a. ERK2) | NM_138957 |
| Inhibitor of nuclear factor kappa B kinase | NM_001190720 |
| subunit beta (IKBKB) | NM_001242778 |
| | NM_001556 |
| | XM_005273491 |
| | XM_005273496 |
| | XM_005273493 |
| | XM_005273498 |
| | XM_011544518 |
| | XM_005273492 |
| | XM_005273490 |
| | XM_005273494 |
| | 12XM_017013396 |
| | XM_011544521 |
| | XM_011544522 |
| | XM_005273495 |
| | XM_011544517 |
| | XM_011544520 |
| | XM_011544519 |
| NFKB inhibitor alpha (NFKBIA) | NM_020529 |
| Interleukin 1 receptor associated kinase 1 | NM_001025242 |
| (IRAK1) | NM_001025243 |
| | NM_001569 |
| | XM_005274668 |
| Mitogen-activated protein kinase 8 | NM_001278547 |
| (MAPK8) (a.k.a. JNK) | NM_001278548 |
| | NM_001323302 |
| | NM_001323320 |
| | NM_001323321 |
| | NM_001323322 |
| | NM_001323323 |
| | NM_001323324 |
| | NM_001323325 |
| | NM_001323326 |
| | NM_001323327 |
| | NM_001323328 |
| | NM_001323329 |
| | NM_001323330 |
| | NM_001323331 |
| | NM_139046 |
| | NM_139049 |
| | XM_024448079 |
| | XM_024448080 |
| Lipopolysaccharide binding protein (LBP) | NM_004139 |
| Mitogen-activated protein kinase kinase 1 | NM_002755 |
| (MAP2K1) (a.k.a. MEK1) | XM_017022411 |
| | XM_011521783 |
| | XM_017022412 |
| | XM_017022413 |
| Mitogen-activated protein kinase kinase 2 | NM_030662 |
| (MAP2K2) (a.k.a. MEK2) | XM_006722799 |
| | XM_017026990 |
| | XM_017026989 |
| | XM_017026991 |
| Mitogen-activated protein kinase kinase 3 | NM_001316332 |
| (MAP2K3) (a.k.a. MEK3) | NM_002756 |
| | NM_145109 |
| | XM_017024859 |
| | XM_005256723 |
| | XM_017024857 |
| | XM_011523959 |
| | XM_017024858 |
| | XM_011523958 |
| Mitogen-activated protein kinase kinase 6 | NM_001330450 |
| (MAP2K6) (a.k.a. MEK6) | NM_002758 |
| | XM_005257516 |
| | XM_011525027 |
| | XM_011525026 |
| | XM_006721975 |
| Mitogen-activated protein kinase kinase | NM_005921 |
| kinase 1 (MAP3K1) (a.k.a. MEKK1) | XM_017009485 |
| | XM_017009484 |
| Mitogen-activated protein kinase kinase | NM_001330431 |
| kinase 3 (MAP3K3) (a.k.a. MEKK3) | NM_001363768 |
| | NM_002401 |
| | NM_203351 |
| | XM_005257378 |
| Mitogen-activated protein kinase kinase | NM_001291958 |
| kinase 4 (MAP3K4) (a.k.a. MEKK4) | NM_001301072 |
| | NM_001363582 |
| | NM_005922 |
| | NM_006724 |
| | XM_017010869 |
| Mitogen-activated protein kinase kinase | NM_001297609 |
| kinase 6 (MAP3K6) (a.k.a. MEKK6) | NM_004672 |
| | XM_017002771 |
| | XM_017002772 |
| Mitogen-activated protein kinase kinase | NM_003188 |
| kinase 7 (MAP3K7) (a.k.a. MEKK7) | NM_145331 |
| | NM_145332 |
| | NM_145333 |
| | XM_006715553 |
| | XM_017011226 |
| MAPK activated protein kinase 2 | NM_004759 |
| (MAPKAPK2) (a.k.a. MK2) | NM_032960 |
| | XM_005273353 |
| | XM_017002810 |
| MYD88, innate immune signal transduction | NM_001172566 |
| adaptor (MYD88) | NM_001172567 |
| | NM_001172568 |
| | NM_001172569 |
| | NM_001365876 |
| | NM_001365877 |
| | NM_002468 |
| Nuclear factor kappa B subunit 1 (NFKB1) | NM_001165412 |
| | NM_001319226 |
| | NM_003998 |
| | XM_024454069 |
| | XM_024454067 |
| | XM_011532006 |
| | XM_024454068 |
| Mitogen-activated protein kinase kinase | NM_003954 |
| kinase 14 (MAP3K14) (a.k.a. NIK) | XM_011525441 |
| Mitogen-activated protein kinase 14 | NM_001315 |
| (MAPK14) (a.k.a. p38) | NM_139012 |
| | NM_139013 |
| | NM_139014 |
| | XM_011514310 |
| | XM_017010300 |
| | XM_017010299 |
| | XM_017010301 |
| | XM_017010304 |
| | XM_017010303 |
| | XM_017010302 |
| | XM_006714998 |
| Eukaryotic translation initiation factor 2 | NM_001135651 |
| alpha kinase 2 (EIF2AK2) (a.k.a. PKR) | NM_001135652 |
| | NM_002759 |
| | XM_011532987 |
| | XM_017004503 |
| AKT serine/threonine kinase 1 (AKT1) | NM_001014431 |
| (a.k.a. RAC) | NM_001014432 |
| | NM_005163 |
| Zinc fingers and homeoboxes 2 (ZHX2) | NM_001362797 |
| (a.k.a. RAF) | NM_014943 |
| | XM_011516932 |
| | XM_005250836 |
| KRAS proto-oncogene, GTPase (KRAS) | NM_001369786 |
| | NM_001369787 |
| | NM_004985 |
| | NM_033360 |
| NRAS proto-oncogene, GTPase (NRAS) | NM_002524 |
| Receptor interacting serine/threonine kinase | NM_001317061 |
| 1 (RIPK1) (a.k.a. RIP) | NM_001354930 |
| | NM_001354931 |
| | NM_001354932 |
| | NM_001354933 |
| | NM_001354934 |
| | NM_003804 |
| | XM_017011405 |
| | XM_006715237 |
| | XM_017011403 |
| | XM_017011404 |
| TNF receptor associated factor 6 (TRAF6) | NM_004620 |
| | NM_145803 |
| | XM_017018220 |
| ZFP36 ring finger protein (ZFP36) (a.k.a. | NM_003407 |
| TTP) | |

An antisense nucleic acid molecule can be fully or partially complementary to all or part of a non-coding region of the coding strand of a nucleotide sequence encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein. Non-coding regions (5′ and 3′ untranslated regions) are the 5′ and 3′ sequences that flank the coding region in a gene and are not translated into amino acids.

Based upon the sequences disclosed herein (e.g., in Table E), one of skill in the art can easily choose and synthesize any of a number of appropriate antisense nucleic acids to target a nucleic acid encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., specificity for any one of the sequences presented in Table E). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein. For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18):12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, INK, MAPK, MEKK 1/4, MEKK4/7, MEKK 3/6, NIK, TRADD, RIP, NF-xB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, R05126766 (CH5126766), PLX7904, and MLN2480).

In some examples, the anti-TNFα agent TNFα inhibitor is a small molecule that inhibits the activity of one of MK2 (PF 3644022 and PHA 767491), INK (e.g., AEG 3482, BI 78D3, CEP 1347, c-JUN peptide, IQ 1S, JIP-1 (153-163), SP600125, SU 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p³⁸ (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), IκB (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 28 nucleotides, about 15 nucleotides to about 26 nucleotides, about 15 nucleotides to about 25 nucleotides, about 15 nucleotides to about 24 nucleotides, about 15 nucleotides to about 22 nucleotides, about 15 nucleotides to about 20 nucleotides, about 15 nucleotides to about 18 nucleotides, about 15 nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and R G M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, a compound of Formulae AA, AA-1, or AA-2 is prepared as shown in Scheme A-1 through the coupling between sulfonamide (A-1) and isocyanate (A-2).

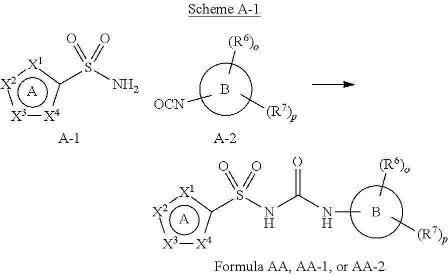

For example, the coupling between A-1 and A-2 can be conducted in the presence of sodium hydride (NaH) (e.g., in THF).

Alternatively, A-1 can be coupled with A-4 (e.g., in the presence of NaH and THF) as shown in Scheme A-2 below to provide a compound of Formulae AA, AA-1, or AA-2 which may be converted into a salt (e.g., a sodium salt such as A-5) upon treatment with base (e.g., NaOH).

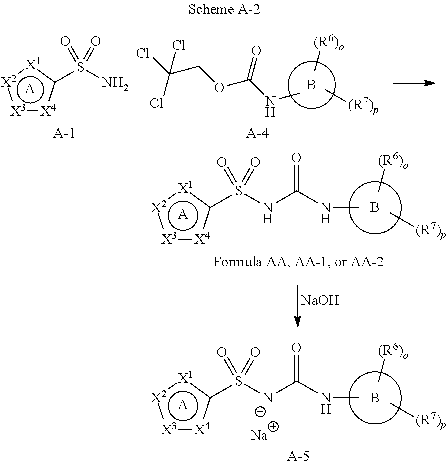

Scheme B (below) depicts one example for the synthesis of a compound of Formula AA or AA-2. Commercially available compound B-1 can be subjected to annulation conditions (e.g., those described in Heterocycles 1987, 26, 2657) to afford compound B-2. Compound B-2 can be sulfonated, whereupon treatment of the intermediate with a chlorination reagent can lead to compound B-3. Compound B-3 can be converted into its corresponding sulfonamide B-4 on treatment with ammonia. Compound B-4 can be coupled with 4-isocyanato-1,2,3,5,6,7-hexahydro-s-indacene to afford compound B-5 which is a compound of Formula AA or AA-2.

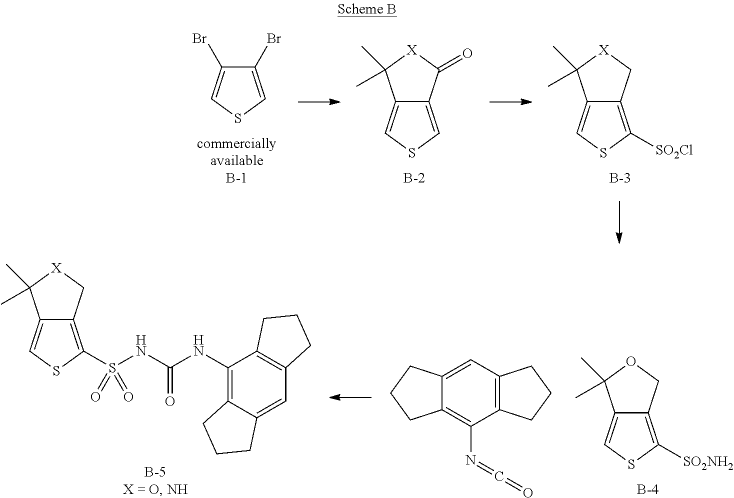

Scheme C (below) depicts another example for the synthesis of a compound of Formula AA or AA-2. Commercially available compound C-1 can be derivatized at the carbonyl group to form compound C-2 (e.g., by means of Grignard addition, or imine formation followed by Grignard addition). Compound C-2 can be sulfonated, whereupon treatment of the intermediate with a chlorination reagent can lead to compound C-3. The hydroxyl group in compound C-2 is optionally protected with a hydroxyl protecting group prior to conversion to C-3. Compound C-3 can be converted into its corresponding sulfonamide C-4 on treatment with ammonia. Compound C-4 can be coupled with 4-isocyanato-1,2,3,5,6,7-hexahydro-s-indacene to afford compound C-5 which is a compound of Formulae AA or AA-2, following the removal of protecting group if present. Compound C-5 is optionally subjected to chiral resolution to yield its two enantiomers which are each a compound of Formulae AA or AA-2.

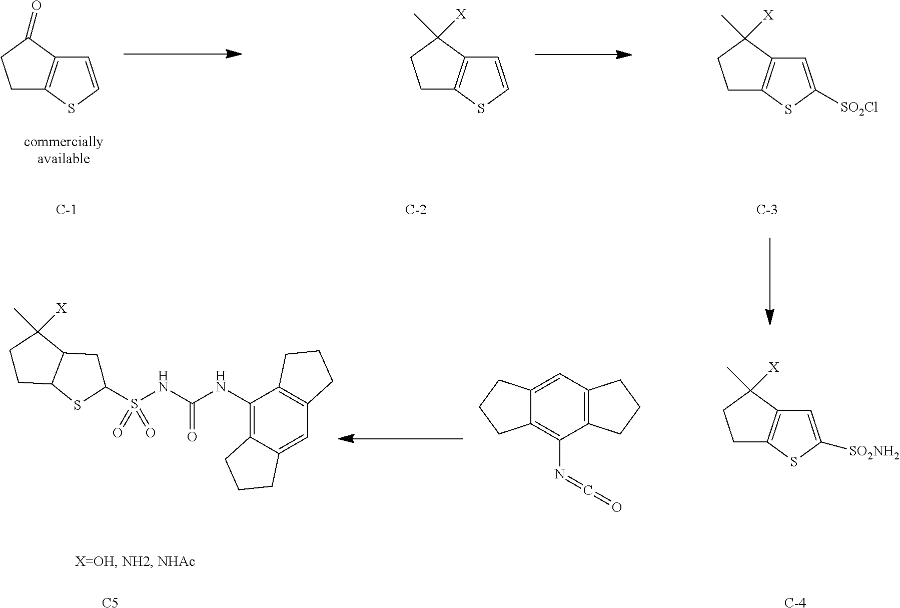

Scheme D depicts yet another example for the synthesis of a compound of Formula AA or AA-2. Commercially available compound D-1 can undergo ring expansion (e.g., via oxidative cleavage of the corresponding enol acetate) to provide a compound of Formula D-2. Sulfonation of D-2, followed by the treatment of the resulting intermediate with a chlorinating agent can provide compound D-3. Treatment of D-3 with ammonia can yield sulfonamide D-4 which can then be coupled with 4-isocyanato-1,2,3,5,6,7-hexahydro-s-indacene to afford compound D-5 which is a compound of Formulae AA or AA-2.

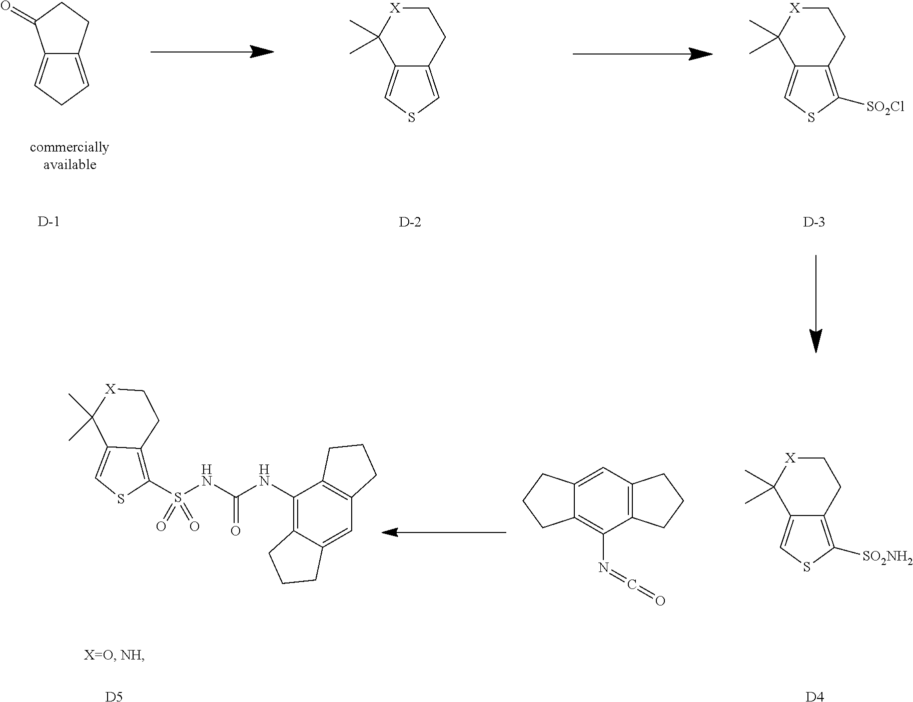

### Preparation of Exemplary Compounds

The following abbreviations have the indicated meanings:

ACN==acetonitrile

BTC═trichloromethyl chloroformate

Boc=t-butyloxy carbonyl

DCM=dichloromethane

DMF=N,N-dimethylformamide

DMSO=dimethyl sulfoxide

DIEA=N,N-diisopropylethylamine

HPLC═high performance liquid chromatography

LC-MS=liquid chromatography-mass spectrometry

Me=methyl

MeOH=methanol

MSA=methanesulfonic acid

NIS=N-iodosuccinimide

NMR=nuclear magnetic resonance

Py=pyridinePd₂(dba)₃=tris(dibenzylideneacetone)dipalladium(O)

PE=petroleum ether
RT=room temperature
Sat.=saturated
TEA=triethylamine
TFA=trifluoroacetic acid
THF=tetrahydrofuran
TLC═thin layer chromatography
TsOH=4-methylbenzenesulfonic acid
UV=ultraviolet
Xphos=2-dicyclohexylphosphino-2′,4′,6′-triisopropylbiphenyl

General

The progress of reactions was often monitored by TLC or LC-MS. The identity of the products was often confirmed by LC-MS. The LC-MS was recorded using one of the following methods.

Method A: Shim-pack XR-ODS, C18, 3×50 mm, 2.5 urn column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), 2 minute total run time.

Method B: Kinetex EVO, C18, 3×50 mm, 2.2 um column, 1.0 uL injection, 1.5 mL/min flow rate, 90-900 amu scan range, 190-400 nm UV range, 10-95% (1.1 min), 95% (0.6 min) gradient with ACN and water (0.5% NH₄HCO₃), 2 minute total run time.

Method C: Shim-pack XR-ODS, C18, 3×50; mm, 2.5 um column, 1.0 uL injection, 1.5 mL/min flow rate, 90-900 amu scan range, 190-400 nm UV range, 5-100% (2.1 min), 100% (0.6 min) gradient with ACN (0.05% TFA) and water (0.05% TFA), 3 minute total run time,

Method D: Kinetex EVO, C18, 3×50 mm, 2.2 um column, 10 uL injection, 1.5 mL/min flow rate, 90-900 amu scan range, 190-400 nm UV % range, 10-95% (21 min), 95% (0.6 min) gradient with ACN and water (0.5% NH₄HCO₃), 3 minute total run time.

Method F: Phenomenex, CHO-7644, Onyx Monolithic C18, 50×4.6 mm, 10.0 uL injection, 1.5 mL/min flow rate, 100-1500 amu scan range, 220 and 254 nm UV detection, 5% with ACN (0.1% TFA) to 100% water (0.1% TFA) over 9.5 min, with a stay at 100% (ACN, 0.10% TFA) for 1 min, then equilibration to 5% (ACN, 0.1% TFA) over 1.5 min.

The final targets were purified by Prep-HPLC. The Prep-HPLC was carried out using the following method.

Method F: Prep-HPLC: Column, XBridge Shield RP18 OBD (19×250 mm, 10 um); mobile phase, Water (10 mmol/L NH₄HCO₃) and ACN, UV detection 254/210 nm.

Method G: Prep-HPLC: Higgins Analytical Proto 200, C18 Column, 250×20 mm, 10 um; mobile phase, Water (0.1% TFA) and ACN (0.1% TFA), UV detection 254/210 nm.

NMR was recorded on BRUKER NMR 300.03 MHz, DUL-C-H, ULTRASHIELD™300, AVANCE II 300 B-ACS™120 or BRUKER NMR 400.13 MHz, BBFO, ULTRASHIELD™400, AVANCE III 400, B-ACS™120.

Scheme of final targets: Schemes below illustrate several conditions used for coupling of sulfonamide and isocyanate to afford amninocarbonyl sulfonamide.

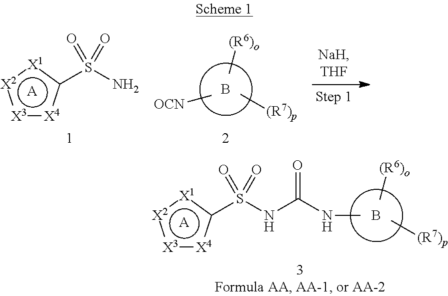

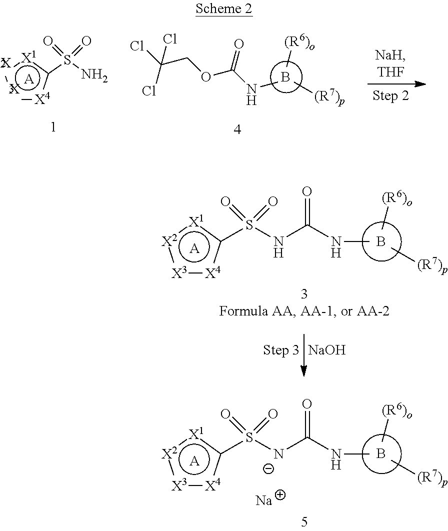

Schemes for the Preparation of Intermediates:

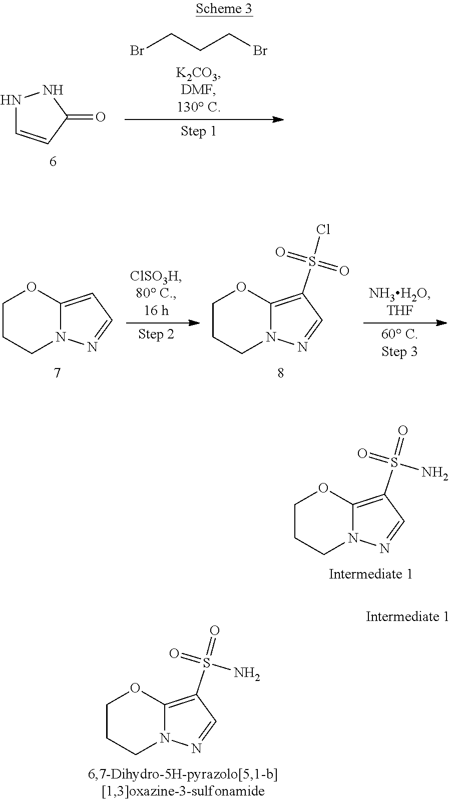 [SMILES: BrCCCBr ; O=c1cc[nH][nH]1 ; c1cc2n(n1)CCCO2 ; O=S(=O)(Cl)c1cnn2c1OCCC2 ; NS(=O)(=O)c1cnn2c1OCCC2 ; NS(=O)(=O)c1cnn2c1OCCC2]

### Step 1: 6,7-Dihydro-5H-pyrazolo[5,1-b][1,3]oxazine

1,2-Dihydro-pyrazol-3-one (5.3 g, 63.1 mmol) and K₂CO₃ (30.5 g, 221 mmol) were heated to 130° C. in DMF (100 mL). 1,3-Dibromopropane (14 g, 69.3 mmol) was added and the mixture was heated for 8 h and then concentrated. The resulting solution was diluted with 50 mL of water and extracted with EtOAc (20 mL×8). The combined organic layer was washed with brine (30 mL), dried over Na₂SO₄, and concentrated in vacuo. The residue was purified by silica gel column (PE/EA=3/1) to give the title compound (3.0 g, 40% yield) as a yellow oil. ¹H NMR (400 MHz, CDCl₃): δ 7.31 (d, J=2.0 Hz, 1H), 5.48 (d, J=2.0 Hz, 1H), 4.28 (t, J=5.2 Hz, 2H), 4.18 (t, J=6.2 Hz, 2H), 2.30-2.23 (m, 2H)

### Step 2: 6,7-Dihydro-5H-pyrazolo[5,1-b][1,3]oxazine-3-sulfonyl chloride

6,7-Dihydro-5H-pyrazolo[5,1-b][1,3]oxazine (3.0 g, 24.2 mmol) was added dropwise to ClSO₃H (200 mL) at 0° C. After being stirred at 80° C. for 16 h, the reaction mixture was added dropwise to a mixture of ice-water/EtOAc (1.5 L/500 mL). The organic layer was separated and the aqueous layer was extracted with EtOAc (300 mL×2). The combined organic layers were washed with brine (300 mL), dried over Na₂SO₄ and concentrated. The residue was washed with PE (50 mL) to give the title compound (3.2 g, y=60%) as a yellow solid. MS-ESI: 223/225 (M+1).

### Step 3: 6,7-Dihydro-5H-pyrazolo[5,1-b][1,3]oxazine-3-sulfonamide

To a solution of 6,7-dihydro-5H-pyrazolo[5,1-b][1,3]oxazine-3-sulfonyl chloride (3.2 g, 14.5 mmol) in THF (100 mL) was added NH₃-H₂O (25%-28% wt.) (50 mL). After being stirred at 60° C. for 16 h, the reaction mixture was concentrated to dryness. The residue was washed with aq. HCl (0.2 M, 30 mL), H₂O (40 mL), and dried to give 6,7-dihydro-5H-pyrazolo[5,1-b][1,3]oxazine-3-sulfonamide (2.3 g, y=78%) as a yellow solid. MS-ESI: 204 (M+1). ¹H NMR (300 MHZ, DMSO-d₆): δ 7.47 (s, 1H), 7.08 (s, 2H), 4.40 (t, J=5.1 Hz, 2H), 4.10 (t, J=6.0 Hz, 2H), 2.25-2.15 (m, 2H).

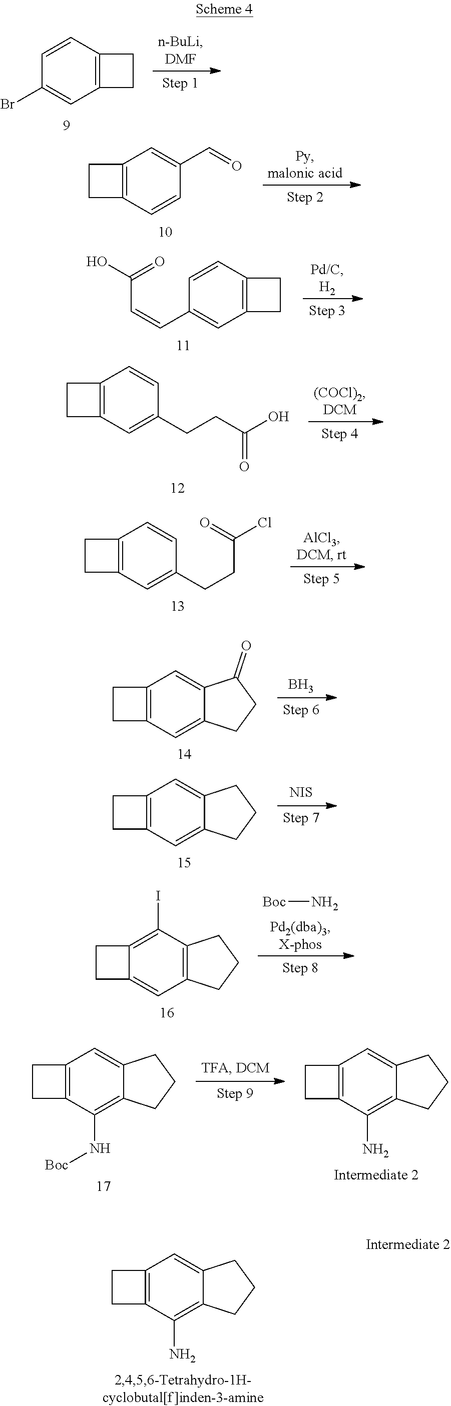

### Step 1: Bicyclo[4.2.0]octa-1(6),2,4-triene-3-carbaldehyde

Into a 500-mL round-bottom flask purged and maintained with an inert atmosphere of nitrogen, was placed 3-bromobicyclo[4.2.0]octa-1(6),2,4-triene (70 g, 382 mmol) in THF (300 mL). This was followed by the addition of n-BuLi (184 mL, 459 mmol) dropwise with stirring at about −70° C. After addition, the reaction mixture was stirred at this temperature for 30 min. To this solution was added DMF (36.3 g, 497 mmol) dropwise with stirring at −70° C. The resulting solution was stirred for 30 min at −70° C. in a liquid nitrogen bath. The reaction was slowly warmed to RT and then quenched by the addition of 100 mL of water. The resulting solution was extracted with 3×200 ml of DCM. The organic layers combined and dried over anhydrous Na₂SO₄, after which the organic layers was concentrated. This resulted in 50 g (98.9%) of the title compound as light yellow oil. MS-ESI. 133 (M+1).

### Step 2: (Z)-3-(bicyclo[4.2.0]octa-1(6),2,4-trien-3-yl)acrylic acid

Into a 250-mL round-bottom flask purged and maintained with an inert atmosphere of nitrogen, was placed bicyclo[4.2.0]octa-1(6),2,4-triene-3-carbaldehyde (17 g, 129 mmol) in pyridine (200 mL), propanedioic acid (19.9 g, 192 mmol) and piperidine (1.1 g, 12.9 mmol). The resulting solution was stirred for overnight at 90° C. in an oil bath. The resulting mixture was concentrated. This resulted in 35 g (crude) of the title compound as a solid. MS-ESI: 173 (M−1).

### Step 3: 3-(Bicyclo[4.2.0]octa-1(6),2,4-trien-3-yl)propanoic acid

Into a 1000-mL round-bottom flask, was placed a solution of 2-(Z or E)-3-[bicyclo[4.2.0]octa-1(6),2,4-trien-3-yl]prop-2-enoic acid (35 g, crude) in MeOH (500 mL). To the solution was added Pd/C (10% wt., 2 g). The flask was evacuated and flushed three times with hydrogen. The resulting solution was stirred for 12 h at RT under an atmosphere of hydrogen. The Pd/C catalysts were filtered out, and the filtrate was concentrated under vacuum. The residue was dissolved in DCM (2000 mL) and then washed with HCl (1 N, 1000 mL), the organic phase was dried over anhydrous Na₂SO₄ then concentrated. This resulted in 21 g (92.6% over 2 steps) of the title compound as a solid. MS-ESI: 175 (M−1).

### Step 4: 3-(Bicyclo[4.2.0]octa-1(6),2,4-trien-3-yl)propanoyl chloride

Into a 250-mL round-bottom flask purged and maintained with an inert atmosphere of nitrogen, was placed 3-[bicyclo[4.2.0]octa-1(6),2,4-trien-3-yl]propanoic acid (10 g, 56.8 mmol) in DCM (100 mL). This was followed by the addition of oxalyl chloride (7.2 g, 56.8 mmol) dropwise with stirring at 0° C. The resulting solution was stirred for 2 h at 0° C. in a water/ice bath. The resulting mixture was concentrated. This resulted in 10 g (crude) of the title compound as light yellow oil which was used in next step without purification.

### Step 5: 1,2,5,6-Tetrahydro-4H-cyclobuta[f]inden-4-one

Into a 100-mL round-bottom flask, was placed 3-[bicyclo[4.2.0]octa-1(6),2,4-trien-3-yl]propanoyl chloride (10 g, crude) in DCM (100 mL). This was followed by the addition of AlCl₃ (6.8 g, 51.4 mmol) in portions at 0° C. over 10 min. The resulting solution was stirred for 1 h at 0° C. in a water/ice bath. The reaction was then quenched by the addition of 200 mL of water. The resulting solution was extracted with 2×100 mL of DCM. The organic layers combined and dried over anhydrous Na₂SO₄, then concentrated. The residue was eluted from a silica gel column with ethyl acetate/petroleum ether (1:20 to 1:15). This resulted in 3.5 g (40.0% over 2 steps) of the title compound as a white solid. ¹H NMR (300 MHz, CDCl₃) δ 7.45 (s, 1H), 7.17 (s, 1H), 3.31-3.22 (m, 4H), 3.18-3.00 (m, 2H), 2.73-2.63 (m, 2H).

### Step 6: 2,4,5,6-Tetrahydro-1H-cyclobuta[f]indene

Into a 500-mL round-bottom flask purged and maintained with an inert atmosphere of nitrogen, was placed 1,2,5,6-tetrahydrocyclobuta[f]inden-4-one (20 g, 126 mmol) in THF (200 mL). This was followed by the addition of BH₃-Me₂S (25.3 mL, 253 mmol, 10 M) dropwise at 0° C. in an ice bath. The resulting solution was stirred for 14 h at 70° C. in an oil bath. The reaction was then quenched by the addition of 20 mL of MeOH. The resulting mixture was concentrated. The residue was eluted from a silica gel column with ethyl acetate/petroleum ether (1:100 to 1:50). This resulted in 15 g (82.3%) of the title compound as colorless oil. ¹H NMR (300 MHz, CDCl₃) δ 6.95 (s, 2H), 3.10 (s, 4H), 2.88 (t, J=7.4 Hz, 4H), 2.04-2.02 (m, 2H).

### Step 7: 3-Iodo-2,4,5,6-tetrahydro-1H-cyclobuta[f]indene

Into a 500-mL round-bottom flask, was placed acetic acid (100 mL), 2,4,5,6-tetrahydro-1H-cyclobuta [f]indene (15 g, 104 mmol) and NIS (35.1 g, 156 mmol). The resulting solution was stirred for 3 h at 50° C. in an oil bath. The resulting solution was diluted with 200 mL of water. The mixture was extracted with 3×100 mL of DCM. The organic layers combined and dried over anhydrous Na₂SO₄, then concentrated. The residue was eluted from a silica gel column with ethyl acetate/petroleum ether (1:100 to 1:80). This resulted in 5.0 g (17.8%) of the title compound as yellow oil.

### Step 8: Tert-butyl (2,4,5,6-tetrahydro-1H-cyclobuta[f]inden-3-yl)carbamate

Into a 250-mL round-bottom flask purged and maintained with an inert atmosphere of nitrogen, was placed 3-iodo-2,4,5,6-tetrahydro-1H-cyclobuta[f]indene (5.0 g, 18.5 mmol) in toluene (100 mL), tert-butyl carbamate (6.5 g, 55.5 mmol), X-phos (900 mg, 1.85 mmol), Pd₂(dba)₃ (800 mg, 0.93 mmol), t-BuOK (6.2 g, 55.5 mmol). The resulting solution was stirred for 14 h at 100° C. in an oil bath. The resulting mixture was concentrated. The residue was eluted from a silica gel column with ethyl acetate/petroleum ether (1:50 to 1:20). This resulted in 3.0 g (83.3%) of the title compound as a white solid. MS-ESI: 260 (M+1). ¹H NMR (300 MHz, CDCl₃) δ 6.72 (s, 1H), 6.13 (br, 1H), 3.26 (d, J=4.5 Hz, 2H), 3.01 (d, J=4.5 Hz, 2H), 2.90 (t, J=7.4 Hz, 2H), 2.75 (t, J=7.4 Hz, 2H), 2.08-2.04 (m, 2H), 1.52 (s, 9H).

### Step 9: 2,4,5,6-Tetrahydro-1H-cyclobuta[f]inden-3-amine

Into a 100-mL round-bottom flask, was placed tert-butyl2,4,5,6-tetrahydro-1H-cyclobuta[f]inden-3-ylcarbamate (3.0 g, 11.6 mmol) in DCM (20 mL). To the solution was added 2,2,2-trifluoroacetic acid (5.0 mL). The resulting solution was stirred for 2 h at RT. The resulting solution was diluted with 50 mL of water. The pH value of the solution was adjusted to 10 with sat. aqueous Na₂CO₃. The resulting solution was extracted with 3×20 mL of DCM. The organic layers were combined, dried over anhydrous Na₂SO₄, and then concentrated. This resulted in 1.5 g (81.4%) of the title compound as a yellow solid. MS-ESI: 160 (M+1).

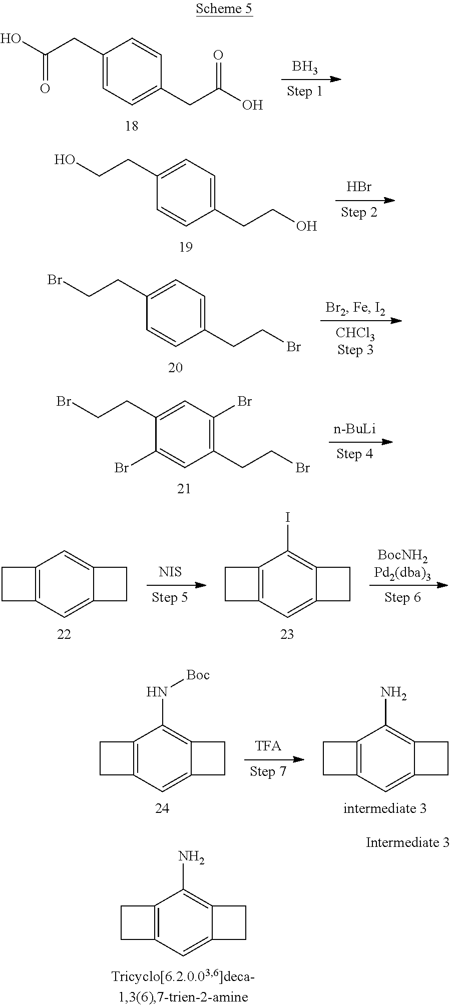

### Step 1: 2,2′-(1,4-Phenylene)bis(ethan-1-ol)

Into a 1.0-L round-bottom flask purged and maintained with an inert atmosphere of nitrogen, was placed 2-[4-(carboxymethyl)phenyl]acetic acid (40 g, 200 mmol) in THF (500 mL). This was followed by the addition of BH₃-Me₂S (60 mL, 600 mmol, 10 M) dropwise with stirring at 0° C. The resulting solution was stirred for 24 h at RT. The reaction was then quenched by the addition of 200 mL of water. The resulting solution was extracted with 2×150 mL of ethyl acetate. The combined organic layers were dried over anhydrous Na₂SO₄ and then concentrated. The resulting residue was eluted from a silica gel column with ethyl acetate/petroleum ether (1:10 to 1:3). This resulted in 28 g (81.8%) of the title compound as brown oil. MS-ESI: 167 (M+1).

### Step 2: 1,4-Bis(2-bromoethyl)benzene

Into a 50-mL round-bottom flask purged and maintained with an inert atmosphere of nitrogen, was placed 2-[4-(2-hydroxyethyl)phenyl]ethan-1-ol (28 g, 168 mmol) in aq. HBr (300 mL, 40% wt.). The resulting solution was stirred for 5 h at 100° C. in an oil bath. The resulting solution was diluted with 500 mL of water and was then extracted with 3×200 mL of DCM. The organic layers were combined and concentrated. This resulted in 40 g (81.4%) of the title compound as a white solid. MS-ESI: 291/293/295 (M+1).

### Step 3: 1,4-Dibromo-2,5-bis(2-bromoethyl)benzene

Into a 500-mL round-bottom flask, was placed 1,4-bis(2-bromoethyl)benzene (30 g, 103 mmol) in trichloromethane (200 mL). To the above solution was added 12 (0.78 g, 3.08 mmol), iron powder (0.75 g, 13.4 mmol), and Br₂ (41 g, 257 mmol). The resulting solution was stirred for 24 h at RT. The reaction was then quenched by the addition of aqueous Na₂SO₃. The resulting solution was extracted with 3×200 mL DCM. The organic layers were combined, dried over anhydrous Na₂SO₄, and then concentrated. This resulted in 40 g (86.6%) of the title compound as a white solid. MS-ESI: 449/451/453 (M+1).

### Step 4: Tricyclo[6.2.0.0^{3,6}]deca-1,3(6),7-triene

Into a 1000-mL round-bottom flask purged and maintained with an inert atmosphere of nitrogen, was placed 1,4-dibromo-2,5-bis(2-bromoethyl)benzene (40 g, 88.9 mmol) in THF (400 mL). This was followed by the addition of n-BuLi (74.7 mL, 187 mmol, 2.5 M) dropwise with stirring at −78° C. in a liquid nitrogen bath. The resulting solution was stirred for 30 min at −78° C. The reaction was then quenched by the addition of aqueous NH₄Cl (300 ml) and extracted with 2×200 mL of DCM and the organic layers was combined and dried over anhydrous Na₂SO₄ then concentrated.

This resulted in 8.0 g (69.1%) of the title compound as a light yellow solid.

### Step 5: 2-Iodotricyclo[6.2.0.0^{3,6}]deca-1,3(6),7-triene

Into a 250-mL round-bottom flask purged and maintained with an inert atmosphere of nitrogen, was placed tricyclo[6.2.0.0^{3,6}]deca-1,3(6),7-triene (8 g, 61.45 mmol) in acetic acid (50 mL) and NIS (20.7 g, 92.2 mmol). The resulting solution was stirred for 3 h at 50° C. in an oil bath. The resulting solution was diluted with 100 mL of water. The reaction was then quenched by the addition of aqueous Na₂SO₃. The resulting solution was extracted with 3×50 mL of DCM. The organic layers were combined, dried over anhydrous Na₂SO₄, and then concentrated. The residue was eluted from a silica gel column with ethyl acetate/petroleum ether (1:100). This resulted in 2.5 g (18.2%) of the title compound as a white solid.

### Step 6: Tert-butyl tricyclo[6.2.0.0^{3,6}]deca-1,3(6),7-trien-2-ylcarbamate

Into a 250-mL round-bottom flask purged and maintained with an inert atmosphere of nitrogen, was placed 2-iodotricyclo[6.2.0.0^{3,6}]deca-1,3(6),7-triene (2.5 g, 9.76 mmol) in toluene (50 mL). To the stirred solution was added tert-butyl carbamate (3.43 g, 29.3 mmol), Pd₂(dba)₃ (447 mg, 0.49 mmol), Xphos (466 mg, 0.98 mmol), and t-BuOK (3.29 g, 29.3 mmol). The resulting solution was stirred for 14 h at 100° C. in an oil bath. The resulting mixture was concentrated. The residue was eluted from a silica gel column with ethyl acetate/petroleum ether (1:50 to 1:30). This resulted in 1.5 g (62.6%) of the title compound as a light yellow solid. MS-ESI: 246 (M+1).

### Step 7: Tricyclo[6.2.0.0^{3,6}]deca-1,3(6),7-trien-2-amine

Into a 50-mL round-bottom flask purged and maintained with an inert atmosphere of nitrogen, was placed tert-butyl N-[tricyclo[6.2.0.0^{3,6}]deca-1,3(6),7-trien-2-yl]carbamate (1.5 g, 6.1 mmol) in DCM (20 mL) and 2,2,2-trifluoroacetic acid (4.0 mL). The resulting solution was stirred for 2 h at RT. The resulting mixture was concentrated. This resulted in 800 mg (90.1%) of the title compound as a brown solid. MS-ESI: 146 (M+1).

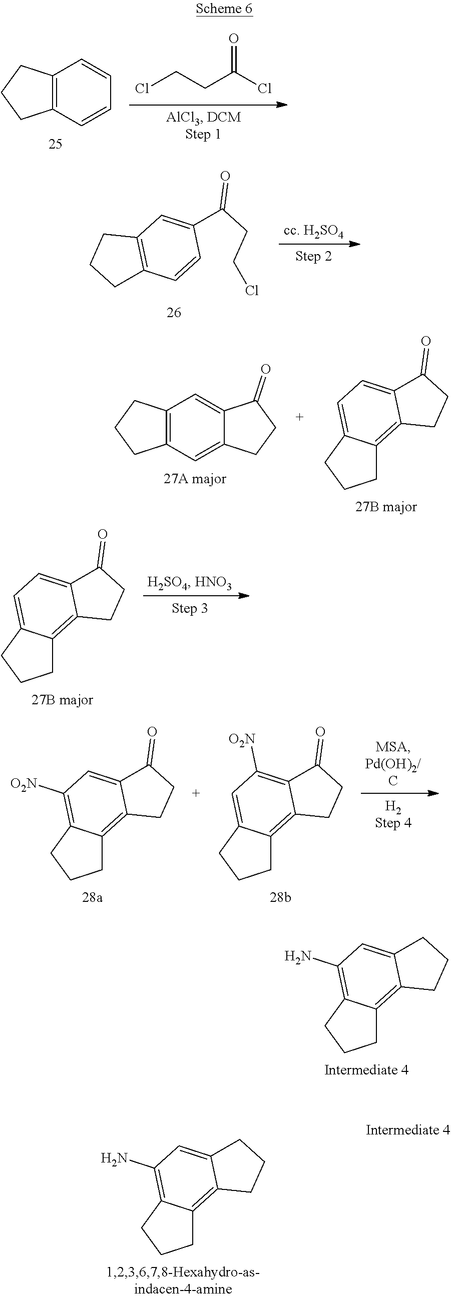

### Step 1: 3-Chloro-1-(2,3-dihydro-1H-inden-5-yl)propan-1-one

Into a 3000-mL round-bottom flask was placed a solution of AlCl₃ (111 g, 834 mmol) in DCM (1200 mL). This was followed by the addition of a solution of 2,3-dihydro-1H-indene (90 g, 762 mmol) and 3-chloropropanoyl chloride (96.3 g, 759 mmol) in DCM (300 mL) dropwise with stirring at −10° C. in 30 min. The resulting solution was stirred for 16 h at RT. Then the reaction mixture was added dropwise to cold HCl aq. (3 N, 1200 mL) over 45 min at −10° C. The resulting solution was extracted with 3×600 mL of DCM and the organic layers were combined, dried over anhydrous Na₂SO₄, and concentrated under vacuum. This resulted in 160.5 g (crude) of the title compound as a yellow solid. The crude product was used in the next step.

### Step 2: 3,5,6,7-tetrahydro-s-indacen-1(2H)-one and 1,6,7,8-tetrahydro-as-indacen-3(2H)-one

Into a 1000-mL round-bottom flask was placed a solution of 3-chloro-1-(2,3-dihydro-1H-inden-5-yl)propan-1-one (160.5 g, 759 mmol) in conc. H₂SO₄ (900 mL). The resulting solution was stirred for 16 h at 55° C., after which the reaction was quenched by adding the reaction mixture carefully to 4500 mL of water/ice. The solids were collected by filtration and dried over infrared lamp for 24 h. The crude mixture was purified by chromatography and eluted with ethyl acetate/petroleum ether (1:100). This resulted in 112.2 g (85%) of 3,5,6,7-tetrahydro-s-indacen-1(2H)-one (27A major) and 10 g (7.6%) of 1,6,7,8-tetrahydro-as-indacen-3(2H)-one (27B minor) as a yellow solid.

27A major: ¹H NMR (400 MHz, DMSO-d₆) δ 7.44 (s, 1H), 7.39 (s, 1H), 3.13-2.79 (m, 6H), 2.70-2.55 (m, 2H), 2.20-1.90 (m, 2H).

27B minor: ¹H NMR (400 MHz, DMSO-d₆) δ 7.49 (d, J=7.7 Hz, 1H), 7.31 (d, J=7.7 Hz, 1H), 3.19-2.98 (m, 4H), 2.93-2.80 (m, 2H), 2.68-2.54 (m, 2H), 2.15-1.95 (m, 2H).

### Step 3: 5-nitro-1,6,7,8-tetrahydro-as-indacen-3(2H)-one and 4-nitro-1,6,7,8-tetrahydro-as-indacen-3(2H)-one

Into a 1000-mL round-bottom flask was placed a solution of 1,6,7,8-tetrahydro-as-indacen-3(2H)-one (9.8 g, 46.5 mmol) in H₂SO₄ (50 mL). Then HNO₃ (5.85 g, 92.9 mmol) was added dropwise over 10 min at 0° C. The resulting solution was stirred for 1 h at 0° C. The reaction mixture was slowly added to a mixture of water/ice (100 mL) and DCM (50 mL) with ice bath cooling. The organic layer was collected, dried over Na₂SO₄ and concentrated under vacuum. This resulted in 11 g (89%) of the title mixture compound as a yellow solid.

### Step 4: 1,2,3,6,7,8-Hexahydro-as-indacen-4-amine

Into a 100-mL round-bottom flask was placed a solution of the mixture of 5-nitro-1,6,7,8-tetrahydro-as-indacen-3(2H)-one and 4-nitro-1,6,7,8-tetrahydro-as-indacen-3(2H)-one (2.17 g, 10 mmol) in MeOH (30 mL). To the solution was added MSA (1.15 g, 12 mmol). Then Pd(OH)₂/C (20% wt., 550 mg) was added. The flask was evacuated and filled three times with hydrogen. The resulting mixture was stirred for 16 h at RT under hydrogen (50 psi). The solids were filtered out and washed with MeOH. The MeOH filtrate was diluted with water (50 mL) and the pH was adjusted to 10.6 with 2 N NaOH. The resulting mixture was filtered and the crude solids were recrystallized from MeOH/water (9:1) with heating. This resulted in 1.38 g (80%) of the title compound as an off-white solid. MS-ESI: 174 (M+1).

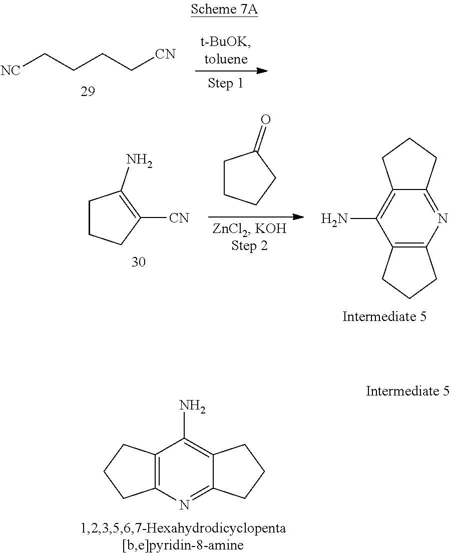 [SMILES: N#CCCCCC#N ; N#CC1=C(N)CCC1 ; O=C1CCCC1 ; Nc1c2c(nc3c1CCC3)CCC2 ; Nc1c2c(nc3c1CCC3)CCC2]

### Step 1: 2-Aminocyclopent-1-ene-1-carbonitrile

Into a 500-mL round-bottom flask purged and maintained with an inert atmosphere of nitrogen, was placed a mixture of adiponitrile (10.8 g, 100 mmol) in toluene (250 mL). The reaction mixture was heated to 65° C., when t-BuOK (112 g, 100 mmol) was added into the solution at 65° C. in portions. The resulting solution was stirring for at 80° C. for 8 h. The reaction was cooled to RT, and then quenched by the addition of 200 mL of water/ice. The solids were collected by filtration. The filter cake was washed with water (100 mL) and hexane (200 mL), then dried under an infra-red lamp. This resulted in 9.18 g (85%) of the title compound as an off-white solid. MS-ESI: 109 (M+1).

### Step 2: 1,2,3,5,6,7-Hexahydrodicyclopenta[b,e]pyridin-8-amine

Into a 250-mL round-bottom flask purged and maintained with an inert atmosphere of nitrogen, was placed 2-aminocyclopent-1-ene-1-carbonitrile (5.0 g, 46.2 mmol) in xylene (125 mL). To the above solution was added cyclopentanone (7.8 g, 93 mmol) and ZnCl₂ (6.9 g, 51 mmol). The resulting solution was stirred for overnight at 140° C. in an oil bath. The resulting solution was diluted with 150 mL of MeOH. Then the solution of KOH (25 mL, 5.0 M) was dropped into the solution. The solids were filtered out. The resulting mixture was concentrated. The residue was dissolved in 250 mL of EtOAc. The solids were collected by filtration. This resulted in 4.2 g (52%) of the title compound as a brown solid. MS-ESI. 175 (M+1).

TABLE S1
The Intermediates in the following Table were prepared using
the similar procedures for converting compound 29 to Intermediate 5
shown in Scheme 7A from appropriated starting materials

| Intermediate | | | Exact Mass |
|---|---|---|---|
| # | Structure | IUPAC Name | [M + H]⁺ |
| Intermediate 6 | 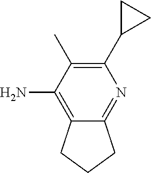 | 2-Cyclopropyl-3- methyl-6,7-dihydro- 5H- cyclopenta[b]pyridin- 4-amine | 189 |

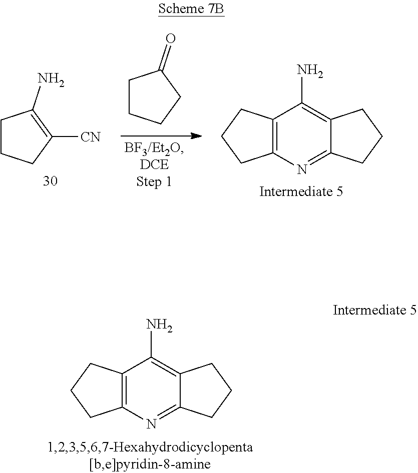 [SMILES: N#CC1=C(N)CCC1 ; O=C1CCCC1 ; Nc1c2c(nc3c1CCC3)CCC2 ; Nc1c2c(nc3c1CCC3)CCC2]

### Step 1: 1,2,3,5,6,7-Hexahydrodicyclopenta[b,e]pyridin-8-amine

Into a 250-mL 3-necked round-bottom flask purged and maintained with an inert atmosphere of nitrogen, was placed a mixture of 2-aminocyclopent-1-enecarbonitrile (5.4 g, 50 mmol) in DCE (125 mL). To this solution was added cyclopentanone (8.4 g, 100 mmol). Then BF₃.Et₂O (46.5% wt., 14.5 g) was added to this solution at 0° C. in an ice bath. The reaction was heat to 75° C. for 6 h, after which the reaction was cooled to RT, quenched by the addition of 100 mL of water/ice, and extracted with DCM (2×50 mL). The aqueous phase was collected, and its pH value was adjusted to 14 with NaOH (6 M) until a solid precipitated. The solids were collected by filtration. The filter cake was washed with water (150 mL) then dried by infra-red drying, this resulted of the title compound (7.0 g, yield 80%, light yellow solid). MS-ESI. 175 (M+1).

TABLE S2
The Intermediates in the following Table were prepared using the similar
procedures for converting compound 30 to Intermediate 5 shown
in Scheme 7B from appropriated starting materials.

| Inter- | | | Exact |
|---|---|---|---|
| mediate | | | Mass |
| # | Structure | IUPAC Name | [M + H]⁺ |
| Inter- mediate 7 | 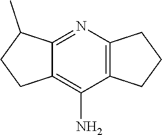 | 3-Methyl-1,2,3,5,6,7- hexahydrodicyclopenta [b,e]pyridin-8-amine | 189 |
| Inter- mediate 8 | 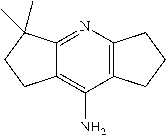 | 3,3-Dimethyl- 1,2,3,5,6,7- hexahydrodicyclopenta [b,e]pyridin-8-amine | 203 |
| Inter- mediate 9 | 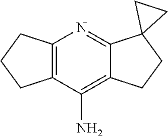 [SMILES: Nc1c2c(nc3c1CCC31CC1)CCC2] | 1′,5′,6′,7′-Tetrahydro- 2′H- spiro[cyclopropane- 1,3′-dicyclopenta[b,e] pyridin]-8′-amine | 201 |

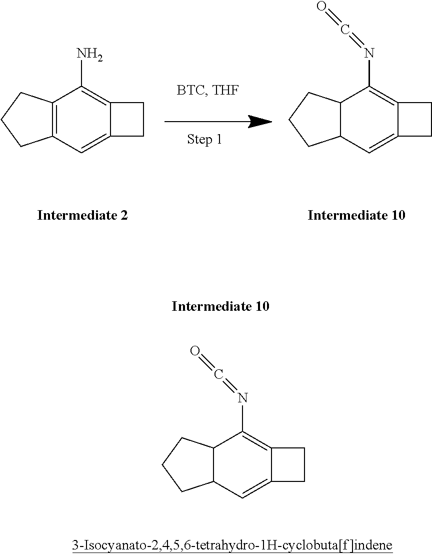 [SMILES: Nc1c2c(cc3c1CC3)CCC2 ; O=C=NC1=C2CCC2=CC2CCCC12 ; O=C=NC1=C2CCC2=CC2CCCC12]

### Step 1: 3-Isocyanato-2,4,5,6-tetrahydro-1H-cyclobuta[f]indene

Into a 50-mL round-bottom flask purged and maintained with an inert atmosphere of nitrogen, was placed 2,4,5,6-tetrahydro-1H-cyclobuta[f]inden-3-amine (30.0 mg, 0.19 mmol) in THE (5.0 mL), TEA, and bistrichloromethyl carbonate (22.4 mg, 0.08 mmol). The resulting solution was stirred for 1 h at 70° C. The resulting mixture was concentrated under vacuum. The crude product was used in the next step directly without further purification.

TABLE S3
The Intermediates in the following Table were prepared
using similar procedure as shown in Scheme 8 above for
converting Intermediate 2 to Intermediate 10.

| Intermediate # | Structure | IUPAC Name |
|---|---|---|
| Intermediate 11 | 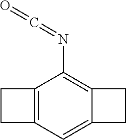 [SMILES: O=C=Nc1c2c(cc3c1CC3)CC2] | 2- Isocyanatotricyclo [6.2.0.03,6]deca- 1,3(6),7-triene |
| Intermediate 12 | 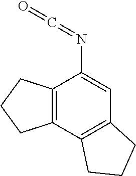 [SMILES: O=C=Nc1cc2c(c3c1CCC3)CCC2] | 4-Isocyanato- 1,2,3,6,7,8- hexahydro-as- indacene |

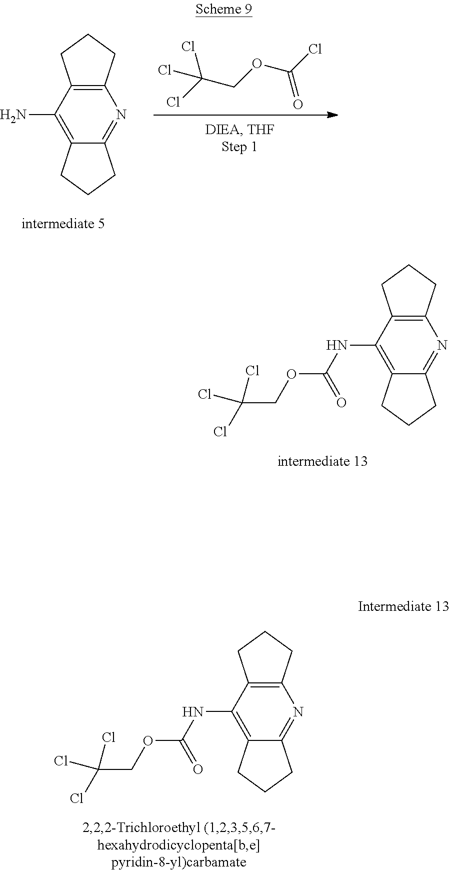 [SMILES: Nc1c2c(nc3c1CCC3)CCC2 ; O=C(Cl)OCC(Cl)(Cl)Cl ; O=C(Nc1c2c(nc3c1CCC3)CCC2)OCC(Cl)(Cl)Cl ; O=C(Nc1c2c(nc3c1CCC3)CCC2)OCC(Cl)(Cl)Cl]

Into a 1-L round-bottom flask purged and maintained with an inert atmosphere of nitrogen, was placed 1,2,3,5,6,7-hexahydrodicyclopenta[b,e]pyridin-8-amine (6.7 g, 38 mmol) in THF (500 mL). To the above solution was added DIEA (9.92 g, 76.9 mmol) dropwise at RT. Then 2,2,2-trichloroethyl chloroformate (16 g, 76.9 mmol) was dropped into the reaction solution at 0° C. The resulting solution was stirred for 16 h at RT. The resulting mixture was concentrated. The residue was eluted from silica gel with EtOAc/hexane (1:4). This resulted in 7.3 g (54.4%) of the title compound as a yellow solid. MS-ESI: 349/351 (M+1).

TABLE S4
The Intermediates in the following Table were prepared using the similar procedures for
converting intermediate 5 to Intermediate 13 shown in Scheme 9 from appropriated starting materials.

| | | | Exact Mass |
|---|---|---|---|
| Intermediate # | Structure | IUPAC Name | [M + H]⁺ |
| Intermediate 14 | 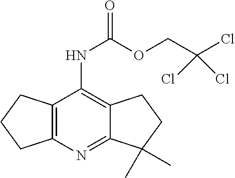 [SMILES: CC1(C)CCc2c1nc1c(c2NC(=O)OCC(Cl)(Cl)Cl)CCC1] | 2,2,2-Trichloroethyl (3,3-dimethyl- 1,2,3,5,6,7- hexahydrodicyclopenta[b,e]pyridin- 8-yl)carbamate | 377/379 |
| Intermediate 15 | 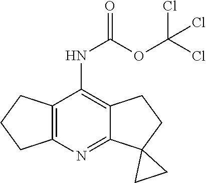 [SMILES: O=C(Nc1c2c(nc3c1CCC31CC1)CCC2)OC(Cl)(Cl)Cl] | Trichloromethyl (1′,5,6,7′- tetrahydro-2′H-spiro[cyclopropane- 1,3′-dicyclopenta[b,e]pyridin]-8′- yl)carbamate | 361/363 |
| Intermediate 16 | 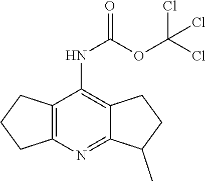 [SMILES: CC1CCc2c1nc1c(c2NC(=O)OC(Cl)(Cl)Cl)CCC1] | Trichloromethyl (3-methyl- 1,2,3,5,6,7- hexahydrodicyclopenta[b,e]pyridin- 8-yl)carbamate | 349/351 |
| Intermediate 17 | 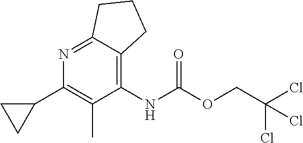 [SMILES: Cc1c(C2CC2)nc2c(c1NC(=O)OCC(Cl)(Cl)Cl)CCC2] | 2,2,2-Trichloroethyl (2- cyclopropyl-3-methyl-6,7-dihydro- 5H-cyclopenta[b]pyridin-4- yl)carbamate | 363/365 |

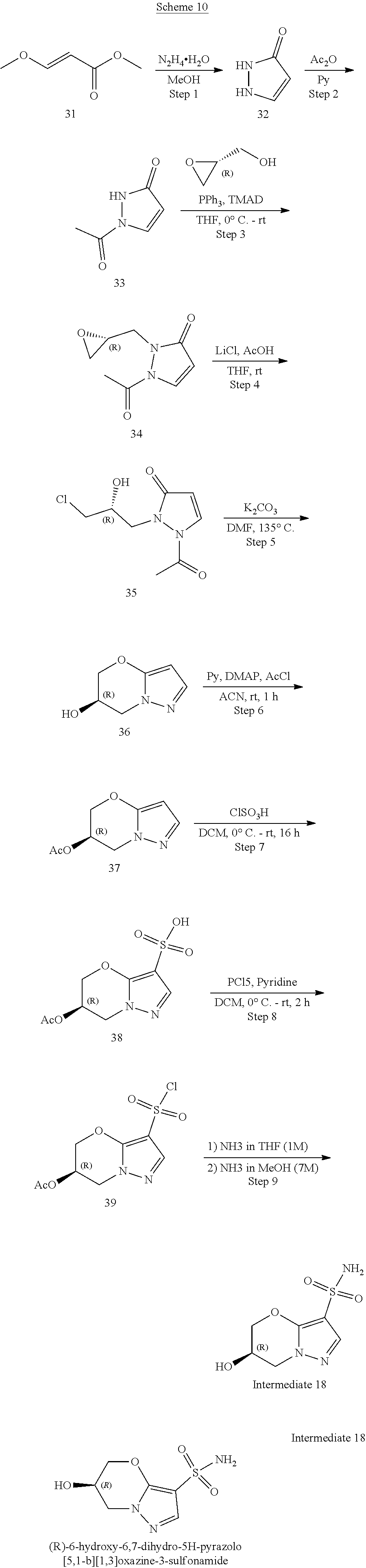

### Step 1: 1,2-Dihydro-3H-pyrazol-3-one

To a 5 L 4-neck flask containing a solution of methyl (E)-3-methoxyacrylate (2000 g, 17.2 mol) in MeOH (2.0 L) was added hydrazine hydrate (921 g, 18.4 mol) dropwise at RT under nitrogen. The resulting mixture was stirred for 90 min at 60° C. under nitrogen. The resulting mixture was concentrated under reduced pressure. This resulted in the title compound (1467 g, 68% wt, yield 69%) as an off-white solid. MS-ESI: 85 (M+1). ¹H NMR (400 MHz, DMSO-d₆) δ 10.36 (br, 2H), 7.18 (q, J=2.5 Hz, 1H), 5.26 (q, J=2.5 Hz, 1H).

### Step 2: 2-Acetyl-1,2-dihydropyrazol-5-one

To a 10 L 4-neck flask containing a solution of 1,2-dihydropyrazol-5-one (1467 g, 68% wt, 11.9 mol) in pyridine (6.0 L) was added Ac₂O (1214 g, 11.9 mol) at RT under nitrogen. The resulting mixture was stirred for 1.5 h at 95° C. under nitrogen. The resulting mixture was concentrated under reduced pressure. The residue was slurry with MeOH (1×3000 mL). The resulting mixture was filtered, the filter cake was washed with MeOH (1×500 mL). The filter cake was dried under reduced pressure. This resulted in the title compound (1630 g, 78% wt, 85%) as an off-white solid. MS-ESI: 127 (M+1). ¹H NMR (400 MHz, DMSO-d₆) δ 8.12 (d, J=3.0 Hz, 1H), 6.00 (d, J=3.0 Hz, 1H), 2.48 (s, 3H), 2.44 (s, 1H).

### Step 3: (R)-2-acetyl-1-(oxiran-2-ylmethyl)-1,2-dihydropyrazol-5-one

To a 10 L 4-neck flask containing a solution of 2-acetyl-1,2-dihydropyrazol-5-one (400 g, 78% wt, 3.17 mol) and R-glycidol (246 g, 3.33 mol) in THF (4.0 L), to the stirred solution was added PPh₃ (915 g, 3.49 mol). To the above mixture was added TMAD (705 g, 3488 mmol) in portions at 0° C. The resulting mixture was stirred for additional 1 h at RT. The resulting mixture was quenched with 1×4.0 L of water. The aqueous layer was extracted with EtOAc (3×1.0 L). The organic layers were combined and washes with brine (1 L), dried over Na₂SO₄ and concentrated under reduced pressure. The crude was slurry in PE/EtOAc (10:1, 16 L) for 4 h. The resulting mixture was filtered. The filter cake was washed with PE/EtOAc (10:1, 1×1000 mL). The filtrate was concentrated under reduced pressure. This resulted in the title compound (470 g, 84% wt, 87%) as an off-white solid. MS-ESI: 183 (M+1). ¹H NMR (400 MHz, DMSO-d₆) δ 8.27 (d, J=3.0 Hz, 1H), 6.27 (d, J=3.0 Hz, 1H), 4.56 (dd, J=11.8, 2.7 Hz, 1H), 4.02 (dd, J=11.8, 6.8 Hz, 1H), 3.40-3.34 (m, 1H), 2.86 (dd, J=5.1, 4.3 Hz, 1H), 2.74 (dd, J=5.1, 2.6 Hz, 1H), 2.54 (s, 3H).

### Step 4: (R)-2-acetyl-1-(3-chloro-2-hydroxypropyl)-1,2-dihydropyrazol-5-one

To a 10 L 3-neck flask was placed a solution of (R)-2-acetyl-1-(oxiran-2-ylmethyl)-1,2-dihydropyrazol-5-one (500 g, 84% wt, 2.74 mol) in THF (2.5 L), to the stirred solution was added AcOH (494 g, 8233 mmol) dropwise and LiCl (186 g, 4391 mmol) in portions at 0° C. under nitrogen. The resulting mixture was stirred for 16 h at RT under nitrogen. The reaction was quenched with water at RT. The aqueous layer was extracted with EtOAc (3×3.0 L). The organic layers were combined and washed with 2×3.0 L of sat. NaHCO₃ and 5.0 L of brine. The organic layer was dried over Na₂SO₄ and concentrated under reduced pressure. This resulted in the title compound (552 g, 82% wt, yield 90%) as an off-white solid. MS-ESI: 219 (M+1). ¹H NMR (400 MHz, CDCl₃) δ 8.10 (d, J=3.0 Hz, 1H), 6.02 (d, J=3.0 Hz, 1H), 4.44 (d, J=5.0 Hz, 2H), 4.29-4.23 (m, 1H), 3.81-3.67 (m, 2H), 2.61 (s, 3H).

### Step 5: (R)-6,7-dihydro-5H-pyrazolo[5,1-b][1,3]oxazin-6-ol

To a 10 L 3-neck flask was placed a solution of (R)-2-acetyl-1-(3-chloro-2-hydroxypropyl)-1,2-dihydropyrazol-5-one (500 g, 82% wt, 2.29 mol) in DMF (5.0 L), to the stirred solution was added K₂CO₃ (948 g, 6.86 mol) under nitrogen. The resulting mixture was stirred for 16 h at 135° C. under nitrogen and concentrated under reduced pressure. The residue was purified by silica gel column chromatography, eluted with DCM/MeOH (20:1) to afford (152 g, yield 58%) of the title compound as an off-white solid. MS-ESI: 141 (M+1). ¹H NMR (400 MHz, MeOH-d₄) δ 7.11 (d, J=2.1 Hz, 1H), 5.31 (d, J=2.1 Hz, 1H), 4.19-4.12 (m, 1H), 4.12-3.98 (m, 3H), 3.90-3.81 (m, 1H).

### Step 6: (R)-6,7-dihydro-5H-pyrazolo[5,1-b][1,3]oxazin-6-yl acetate

To a stirred solution of (R)-6,7-dihydro-5H-pyrazolo[5,1-b][1,3]oxazin-6-ol (55.0 g, 392 mmol) in MeCN (825 mL) in a 2-L 3-necked round-bottom flask under nitrogen was added pyridine (93.1 g, 1.18 mol) and DMAP (4.79 g, 39.2 mmol). This was followed by the addition of acetyl chloride (43.1 g, 549 mmol) dropwise with stirring at 0° C. The resulting solution was stirred for 2 h at RT. LCMS showed reaction was completed. The resulting mixture was concentrated directly. The residue was eluted from silica gel with EtOAc/PE (1:4). This resulted in 58 g (81%) of the title compound as a light yellow solid. MS-ESI: 183 (M+1). ¹H NMR (400 MHz, CDCl₃) δ 7.39 (d, J=2.0 Hz, 1H), 5.57 (d, J=2.0 Hz, 1H), 5.45-5.36 (m, 1H), 4.49-4.41 (m, 1H), 4.40-4.27 (m, 2H), 4.24 (dd, J=12.1, 1.5 Hz, 1H), 2.14 (s, 3H).

### Step 7: (R)-6-acetoxy-6,7-dihydro-5H-pyrazolo[5,1-b][1,3]oxazine-3-sulfonic acid

To a stirred solution of (R)-6,7-dihydro-5H-pyrazolo[5,1-b][1,3]oxazin-6-yl acetate (58.0 g, 318 mmol) in DCM (120 mL) in a 2-L 3-necked round-bottom flask under nitrogen was added chlorosulfonic acid (81.3 g, 700 mmol) dropwise at 0° C. The resulting solution was stirred for 12 h at RT. LCMS showed the conversion was completed. The reaction mixture was used directly in the next step. MS-ESI: 263 (M+1).

### Step 8: (R)-3-(chlorosulfonyl)-6,7-dihydro-5H-pyrazolo[5,1-b][1,3]oxazin-6-yl acetate

To a stirred solution of (R)-6-acetoxy-6,7-dihydro-5H-pyrazolo[5,1-b][1,3]oxazine-3-sulfonic acid in DCM (crude from step 7) in a 2-L 3-necked round-bottom flask under nitrogen was added pyridine (55.1 g, 696 mmol) dropwise at 0° C. To this was added PCl₅ (144 g, 696 mmol) in portions at 0° C. The resulting solution was stirred for 2 h at RT. The reaction was then quenched by the addition of 1 L of water/ice. The resulting solution was extracted with 3×300 mL of EtOAc. The resulting mixture was washed with 2×500 ml of NaHCO₃ and 1×500 mL of H₂O. The resulting mixture was washed with 1×500 mL of NaCl (aq.). The mixture was dried over anhydrous Na₂SO₄ and concentrated under vacuum. This resulted in 76 g (crude) of the title compound as a light yellow solid. MS-ESI: 281/283 (M+1).

### Step 9: (R)-6-hydroxy-6,7-dihydro-5H-pyrazolo[5,1-b][1,3]oxazine-3-sulfonamide

To a stirred solution of NH₃ in THF (1 M, 730 ml) in a 3-L round-bottom flask was added (R)-3-(chlorosulfonyl)-6,7-dihydro-5H-pyrazolo[5,1-b][1,3] oxazin-6-yl acetate (73.0 g) in several batches. The flask was then filled with NH₃ (balloon). The resulting solution was stirred overnight at 40° C. in an oil bath. After reaction completed, the solids were filtered out. The filtrate was concentrated. The residue was diluted with NH₃ (7 M in MeOH, 730 mL). The resulting solution was stirred for 2 h at RT. LC showed the reaction was complete. The MeOH solution was concentrated under reduced pressure to a final volume of about 100 mL. Et₂O (360 ml) was charged to the resulting solution and kept stirring for 30 min. The mixture was filtered, and the cake washed with Et₂O (100 mL). The white solid was dried under vacuum. This resulted in 37 g (53% for 3 steps) of the title compound as a white solid. MS-ESI: 220 (M+1). ¹H NMR (300 MHz, DMSO-d₆) δ 7.48 (s, 1H), 7.06 (s, 2H), 5.64 (d, J=2.3 Hz, 1H), 4.30 (s, 3H), 4.30-4.18 (m, 1H), 4.00-3.90 (m, 1H).

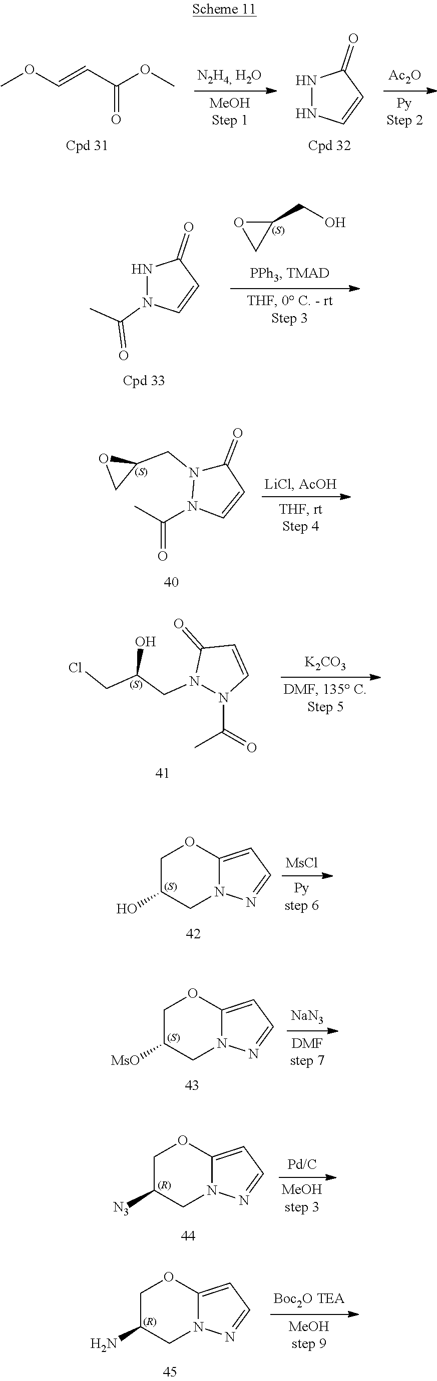

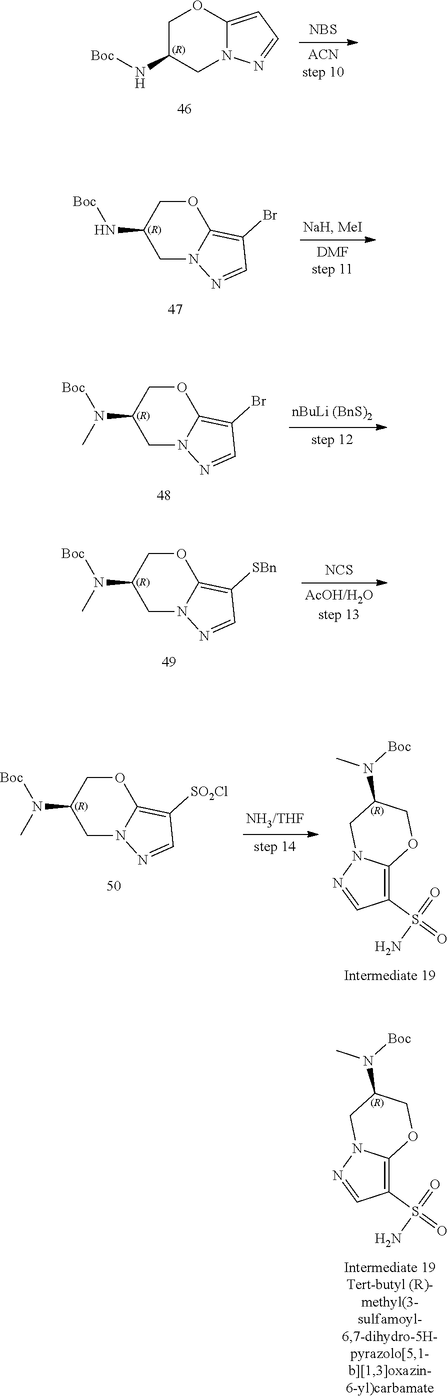

Steps 1-5 used similar procedures for converting compound 31 to compound 36 shown in Scheme 10 to afford compound 42 from compound 31. MS-ESI. 141 (M+1).

### Step 6: (S)-6,7-dihydro-5H-pyrazolo[5,1-b][1,3]oxazin-6-yl methanesulfonate

To a stirred solution of (S)-6,7-dihydro-5H-pyrazolo[5,1-b][1,3]oxazin-6-ol (40 g, 285 mmol) in pyridine (280 mL) in a 500 mL 3-neck flask under nitrogen was added MsCl (39 g, 343 mmol) dropwise at RT. The resulting mixture was stirred for 30 min at RT. The resulting mixture was concentrated under reduced pressure. The crude was diluted with 500 mL of water. The resulting mixture was extracted with EtOAc (3×300 mL). The combined organic layers were washed with water (3×200 mL) and brine 100 mL, dried over anhydrous Na₂SO₄. After filtration, the filtrate was concentrated under reduced pressure. This resulted in the title compound (57 g, 92%) as an off-white solid. MS-ESI: 219 (M+1).

### Step 7: (R)-6-azido-6,7-dihydro-5H-pyrazolo[5,1-b][1,3]oxazine

To a stirred solution of (S)-6,7-dihydro-5H-pyrazolo[5,1-b][1,3]oxazin-6-yl methanesulfonate (46 g, 211 mmol) in DMF (313 mL) in a 1 L 3-neck flask under nitrogen was added NaN₃ (21 g, 316 mmol) at RT. The resulting mixture was stirred for 6 h at 80° C. LCMS showed reaction was complete. This was used directly in the next step. MS-ESI: 166 (M+1).

### Step 8: (R)-6,7-dihydro-5H-pyrazolo[5,1-b][1,3]oxazin-6-amine

To a 1 L 1-neck flask placed solution of (R)-6-azido-6,7-dihydro-5H-pyrazolo[5,1-b][1,3]oxazine (46 g, 279 mmol) in MeOH (313 mL), to the stirred solution was added Pd/C (10% wt., 10 g) under nitrogen in a 1 L round-bottom flask. The mixture was hydrogenated at RT for 5 h under hydrogen atmosphere using a hydrogen balloon. LCMS showed reaction was complete. The mixture was filtered through a pad of Celite and concentrated. This was used directly in the next step. MS-ESI: 140 (M+1).

### Step 9: Tert-butyl (R)-(6,7-dihydro-5H-pyrazolo[5,1-b][1,3]oxazin-6-yl)carbamate

To a 1 L 3-neck flask placed solution of (R)-6,7-dihydro-5H-pyrazolo[5,1-b][1,3]oxazin-6-amine(46 g, 331 mmol) in DMF (313 mL) from last step was added MeOH (313 mL), then to the stirred solution was added TEA (50 g, 496 mmol), followed by the addition of di-tert-butyl dicarbonate (79 g, 364 mmol) at RT under nitrogen. The resulting mixture was stirred for 16 h at RT and then quenched with 500 mL of water. The resulting mixture was extracted with EtOAc (3×300 mL). The combined organic layers were washed with H₂O (2×600 mL) and brine (1×600 mL) and dried over anhydrous Na₂SO₄. After filtration, the filtrate was concentrated under reduced pressure. This resulted in the title compound (45.9 g, 91%, for 3 steps) as an off-white solid. MS-ESI: 240 (M+1).

### Step 10: Tert-butyl (R)-(3-bromo-6,7-dihydro-5H-pyrazolo[5,1-b][1,3]oxazin-6-yl)carbamate

To a stirred solution of tert-butyl (R)-(6,7-dihydro-5H-pyrazolo[5,1-b][1,3] oxazin-6-yl)carbamate (140 g, 585 mmol) in MeCN (2.1 L) in a 3 L 3-neck flask under nitrogen was added NBS (115 g, 644 mmol) at 0° C. The resulting mixture was stirred for 2 h at RT. The resulting mixture was diluted with 2000 mL of water and extracted with EtOAc (3×800 mL). The combined organic layers were washed with brine (1×800 mL) and dried over anhydrous Na₂SO₄. After filtration, the filtrate was concentrated under reduced pressure. This resulted in the title compound (167 g, 90%) as an off-white solid. MS-ESI: 318/320 (M+1).

### Step 11: Tert-butyl (R)-(3-bromo-6,7-dihydro-5H-pyrazolo[5,1-b][1,3]oxazin-6-yl)(methyl)carbamate

To a stirred solution of tert-butyl(R)-(3-bromo-6,7-dihydro-5H-pyrazolo-[5,1-b][1,3]oxazin-6-yl)carbamate (170 g, 534 mmol) in DMF (1.19 L) in a 3 L 3-neck flask under nitrogen was added sodium hydride (60% oil dispersion, 26 g, 650 mmol) in portions at 0° C. The mixture was stirred for 1 h at 0° C. Then Mel (379 g, 2.67 mol) was added and the mixture was allowed to warm to RT and stirred for 2 h. The reaction mixture was quenched by water and extracted with EtOAc (3*800 mL), the organic layers were washed with H₂O (3×500 mL) and brine (1×800 mL), dried over anhydrous Na₂SO₄. After filtration, the filtrate was concentrated under reduced pressure. The residue was purified by silica gel column chromatography, eluted with PE/EtOAc (8:1) to afford the title compound (153 g, 86%) as an off-white solid. MS-ESI: 332/334 (M+1).

### Step 12: Tert-butyl(R)-(3-(benzylthio)-6,7-dihydro-5H-pyrazolo[5,1-b][1,3]oxazin-6-yl)(methyl) carbamate

To a stirred solution of tert-butyl(R)-(3-bromo-6,7-dihydro-5H-pyrazolo-[5,1-b][1,3]oxazin-6-yl)(methyl) carbamate (130 g, 391 mmol) in THF (1.3 L) in a 3 L 3-neck flask under nitrogen was added n-BuLi (188 mL, 470 mmol, 2.5 mol/L) dropwise at −78° C. The resulting mixture was stirred for 1 h at −78° C. To the above mixture was added bis(phenylmethyl) disulfide (145 g, 587 mmol) in THF (300 mL) dropwise at −78° C. The resulting mixture was stirred for additional 2 h at RT. The reaction was quenched by the addition of sat. NH₄Cl (aq.) (500 mL) at RT. The resulting mixture was extracted with EtOAc (3×500 mL). The combined organic layers were washed with brine (1×800 mL), dried over anhydrous Na₂SO₄. After filtration, the filtrate was concentrated under reduced pressure. The residue was purified by silica gel column chromatography, eluted with PE/EtOAc (6:1) to afford the title compound (106 g, 72%) as an off-white solid. MS-ESI: 376 (M+1).

### Step 13: Tert-butyl (R)-(3-(chlorosulfonyl)-6,7-dihydro-5H-pyrazolo[5,1-b][1,3]oxazin-6-yl)(methyl) carbamate

To a stirred solution of tert-butyl (R)-(3-(benzylthio)-6,7-dihydro-5H-pyrazolo[5,1-b][1,3]oxazin-6-yl) (methyl)carbamate (110 g, 293 mmol) in AcOH (3.67 L)/H₂O (1.83 L) in a 10 L 3-neck flask under nitrogen was added NCS (155 g, 1.17 mol) in portions at 0° C. The resulting mixture was stirred for 1 h at RT. The reaction was quenched with water/ice at RT. The resulting mixture was extracted with MTBE (3×1 L). The combined organic layers were washed with water (2×1.0 L), NaHCO₃(L) and brine (1×1 L), dried over anhydrous Na₂SO₄. After filtration, the filtrate was concentrated under reduced pressure. This resulted in the title compound (80 g, crude) as a yellow solid. MS-ESI: 374/376 (M+Na).

### Step 14: Tert-butyl (R)-methyl(3-sulfamoyl-6,7-dihydro-5H-pyrazolo[5,1-b][1,3]oxazin-6-yl)carbamate

To a stirred solution of tert-butyl (R)-(3-(chlorosulfonyl)-6,7-dihydro-5H-pyrazolo [5,1-b][1,3]oxazin-6-yl) (methyl)carbamate (80 g, 227 mmol) in NH₃ in THF (1 M, 800 mL, 800 mmol) in a 2 L 3-neck flask under nitrogen. The flask was then filled with NH₃ (balloon). The resulting mixture was stirred for overnight at 60° C. The reaction mixture was quenched by water and extracted with MTBE (3×800 mL), the organic layers were washed with H₂O (3×500 mL) and brine (1×800 mL), dried over anhydrous Na₂SO₄. After filtration, the filtrate was concentrated under reduced pressure. The precipitated solid was slurry with MTBE (1×100 mL). This resulted in the title compound (36 g, 40.0% for 2 steps) as an off-white solid. MS-ESI: 333 (M+1).

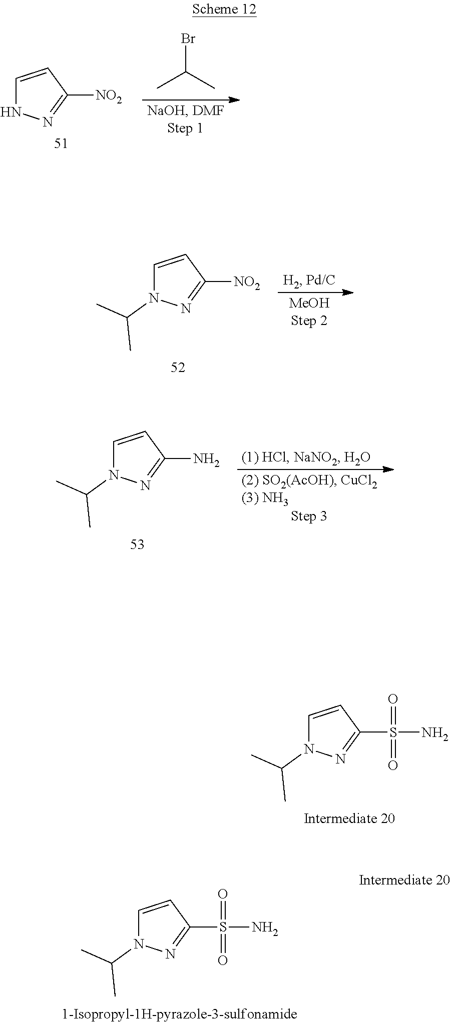

### Step 1: 1-Isopropyl-3-nitro-1H-pyrazole

To a stirred solution of 3-nitro-1H-pyrazole (10 g, 88.4 mmol) in DMF (100 mL) in a 250-mL round-bottom flask under nitrogen was added NaH (60% wt. dispersion in mineral oil, 3.9 g, 97.5 mmol) in portions at 0° C. The resulting solution was stirred for 0.5 h at 0° C. This was followed by the addition of 2-bromopropane (14.1 g, 1.15 mol) dropwise with stirring at 0° C. in 10 min. The resulting solution was stirred for 16 h at RT and then was quenched by the addition of 100 mL of water. The resulting solution was extracted with 3×100 mL of EtOAc. The organic layers were combined and dried over anhydrous Na₂SO₄, then concentrated under vacuum. The residue was eluted from silica gel with a gradient of EtOAc/PE (1:5 to 1:3). This resulted in 11.8 g (86%) of the title compound as yellow oil. MS-ESI: 156 (M+1).

### Step 2: 3-Amino-1-(propan-2-yl)-1H-pyrazole

To a stirred solution of 1-isopropyl-3-nitro-1H-pyrazole (10.8 g, 69.6 mmol) in MeOH (100 mL) in a 250-mL round-bottom flask under nitrogen was added Pd/C (10% wt., 1.5 g) in portions at 0° C. The flask was evacuated and flushed three times with hydrogen. The mixture was stirred for 24 h at RT under an atmosphere of hydrogen with a balloon. The solids were filtered out. The resulting filtrate was concentrated under vacuum. This resulted in 7.27 g (83%) of the title compound as yellow oil. MS-ESI: 126.1 (M+1).

### Step 3: 1-Isopropyl-1H-pyrazole-3-sulfonamide

To a stirred solution of 1-isopropyl-1H-pyrazol-3-amine (2.5 g, 20.0 mmol) in HCl (6 M, 30 mL) in a 250-mL 3-necked round-bottom flask under nitrogen was added a solution of NaNO₂ (1.66 g, 24.0 mmol) in H₂O (5 mL) dropwise with stirring at 0° C., the solution was stirred for 30 min, this solution was assigned as solution A. Then CuCl₂ (1.34 g, 10.0 mmol) was added to a 250-mL single necked round-bottom flask with AcOH (40 mL) as the solvent. Then SO₂ (g) was bubbled to the reaction mixture with stirring at RT for 20 min, this solution was assigned as solution B. To the solution B was added solution A dropwise with stirring at 0° C. The resulting solution was stirred for 2 h at RT. The reaction mixture was diluted with 100 mL of water, extracted with 3×100 mL of DCM and the organic layers were combined and dried over anhydrous Na₂SO₄. To the crude DCM solution (˜300 mL) was bubbled NH₃ (g) with stirring at 0° C. for 10 min. The resulting solution was stirred for 2 h at RT. The solution was concentrated under vacuum. The crude product was eluted from silica gel with EtOAc/PE (1:1). This resulted in 2.61 g (69%) of the title compound as yellow oil. MS-ESI: 188 (M−1).

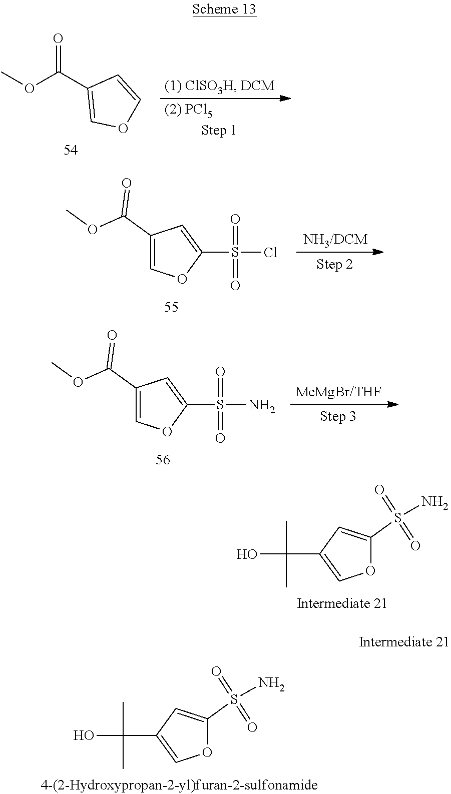

Steps 1-2 used similar procedures for converting compound 7 to Intermediate 1 shown in Scheme 3 to afford compound 54 from compound 56. MS-ESI: 204 (M−1)

### Step 3: 4-(2-Hydroxypropan-2-yl)furan-2-sulfonamide

To a stirred solution of methyl 5-sulfamoylfuran-3-carboxylate (4.55 g, 22.2 mmol) in THF (300 mL) in a 500-mL 3-necked round-bottom flask under nitrogen was added MeMgBr/THF (3 M, 74 mL, 222 mmol) dropwise at 0° C. The resulting solution was stirred for 16 h at RT. The reaction was then quenched with 50 mL of NH₄Cl (aq). The resulting solution was extracted with 3×100 ml of EtOAc and the organic layers were combined and dried over Na₂SO₄ and concentrated under vacuum. The residue was eluted from silica gel with DCM/MeOH (60:1). This resulted in 2.46 g (54.1%) of the title compound as a light yellow solid. MS-ESI: 204 (M−1).

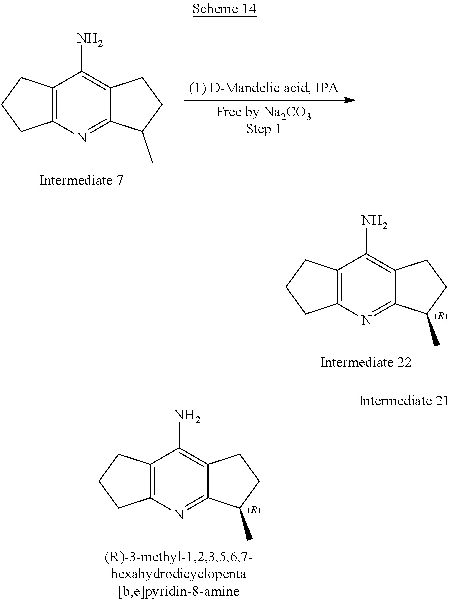

### Step 1: (R)-3-Methyl-1,2,3,5,6,7-hexahydrodicyclopenta[b,e]pyridin-8-amine

A stirred solution of 3-methyl-1,2,3,5,6,7-hexahydrodicyclopenta[b,e]pyridin-8-amine (47 g, 250 mmol) in isopropyl alcohol (423 mL) was heated to 80° C. in a 1 L 3-neck flask with condenser and a stirring bar. The solution was kept stirring for 0.5 h. The solution was cooled to about 50° C. The solution was filtered at that temperature though a Buchner funnel, to remove insoluble impurities. The solid was washed with isopropyl alcohol (3×10 mL). The filtrate was transfer to a round bottle and concentrated to 350 g. The solution was transferred to a 2 L 3-necked round-bottom flask equipped with a mechanical stir and reflux condenser in an oil bath. The round bottle was washed with 27 g isopropyl alcohol to remove all the material to the 2 L 3-necks flask. The resulting solution was heated to 80° C. and a solution of (R)-(−)-Mandelic acid (38 g, 250 mmol) in isopropyl alcohol (188 mL) was added dropwise at that temperature. The resulting mixture was stirred at 80° C. for 5 minute. The system was cooled in 5° C. step and a small amount of the seed crystals of the product was added. If the seed dissolved, repeat the above operation. When the system was cooled to 65° C., the seed was undissolved and crystals started to grow, the system turned to cloudy slowly, the solution was stirred for 30 minute at this temperature. From then on, the solution was stirred and maintained for 30 minute while the temperature was cooled in 5° C. steps until the system temperature was 40° C. Turn off the heating switch of oil bath and the mixture was slowly cooled to 28° C. After 16 h, the ee value of solid was monitored (96% ee). The solid was collected by filtered. The resulting solid was slurry in isopropyl alcohol (180 mL) for 1 h and filtrated again. The filtrate was decanted, and the precipitate was washed with chromatographic isopropyl alcohol (30 mL) to give the title compound as a white solid (35 g, 40.5% yield, 98% ee, which contain 13.8% IPA). The solid was dissolved in water (420 mL) and concentrated to afford 29.2 g white solid (KF=5.1%). The mother liquid was combined and concentrated to afford 56.7 g S-isomer (75% ee, contain 17.8% IPA) as a foam. Then 26.9 g (R) salt was freed by aq. Na₂CO₃ (4 M, 500 mL) and exacted with 3×300 mL EA, then the organic phase was washed with 500 mL of brine and dried over anhydrous Na₂SO₄ and concentrated under vacuum. This resulted in 14.3 g (38% yield, 98% ee, LCAP=96%) of the title compound as a white solid. MS-ESI: 189 (M+1). Chiral analysis method: Column, CHIRALPAK IC 4.6*150 mm, 3 μm. Mobile phase: Hex (0.1% DEA):IPA=85:15. Column Temperature: 30° C. Flow Rate: 1.0 mL/min. Monitor: UV 254 nm. Intermediate 161 is the first peak with a retention time of 5.8 min. The S enantiomer is the second peak with a retention time of 7.0 min.

TABLE 9
The Intermediates in the following table was prepared
using similar procedure as shown in Scheme 6 above for
converting compound 27B to Intermediate 4 starting from 27A.

| | | | Exact |
|---|---|---|---|
| Intermediate | | IUPAC | Mass |
| # | Structure | Name | [M + H]⁺ |
| Intermediate 23 | 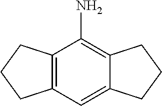 [SMILES: Nc1c2c(cc3c1CCC3)CCC2] | 1,2,3,5,6,7- Hexahydro- s-indacen-4- amine | 174 |

TABLE 10
The Intermediates in the following table were prepared using the
similar procedures for converting Intermediate 2 to Intermediate 10
shown in Scheme 8 from appropriated starting materials.

| Intermediate | | IUPAC |
|---|---|---|
| # | Structure | Name |
| Intermediate 24 | 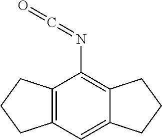 [SMILES: O=C=Nc1c2c(cc3c1CCC3)CCC2] | 4- Isocyanato- 1,2,3,5,6,7- hexahydro- s-indacene |

TABLE 11
The Intermediates in the following Table were
prepared using the similar procedures for converting
Intermediate 5 to Intermediate 13 shown in
Scheme 9 from appropriated starting materials.

| Inter- | | | |
|---|---|---|---|
| mediate | | IUPAC | Exact Mass |
| # | Structure | Name | [M + H]⁺ |
| Inter- mediate 25 | 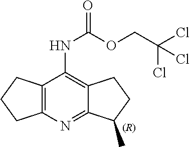 | 2,2,2- trichloroethyl (R)-(3- methyl- 1,2,3,5,6,7- hexahydro- dicyclopenta [b,e]pyridin- 8- yl)carbamate | 363/365/367 |

### Step 1: 1-Ethyl-3-nitro-1-pyrazole

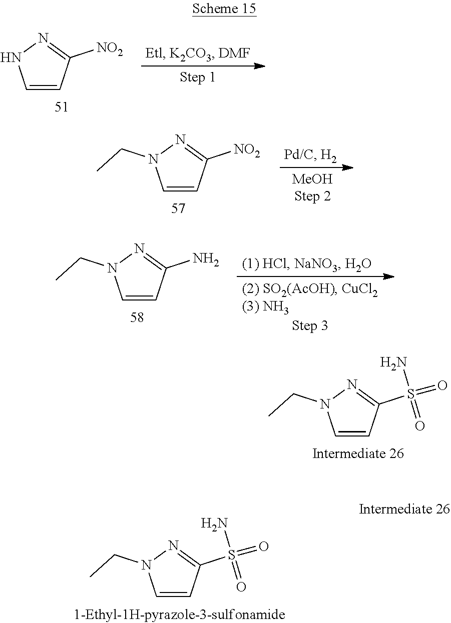

To a stirred solution of 3-nitro-1H-pyrazole (20 g, 177 mmol) in DMF (100 mL) in a 250-mL round-bottom flask were added K₂CO₃ (49 g, 354 mmol) in portions at RT, followed by the addition of iodoethane (55 g, 354 mmol) dropwise at RT. The reaction mixture was stirred for 16 h at 80° C. in an oil bath. The reaction mixture was diluted with 200 mL of H₂O. The mixture was extracted with 3×200 mL of EtOAc and the organic layers were combined and dried over anhydrous Na₂SO₄. The mixture was concentrated under vacuum. The residue was eluted from silica gel with EtOAc/PE (1:4). This resulted in 22.6 g (91%) of the title compound as yellow oil. MS-ESI: 142 (M+1). ¹H NMR (400 MHz, DMSO-d₆) δ 8.06 (d, J=2.5 Hz, 1H), 7.04 (d, J=2.5 Hz, 1H), 4.27 (q, J=7.3 Hz, 2H), 1.42 (t, J=7.3 Hz, 3H).

### Step 2: 1-Ethyl-1H-pyrazol-3-amine

To a stirred solution of 1-ethyl-3-nitro-1H-pyrazole (22 g, 156 mmol) in MeOH (100 mL) in a 250-mL round-bottom flask under nitrogen was added Pd/C (10% wt., 2.2 g) in portions at 0° C. The flask was evacuated and refilled three times with hydrogen. The reaction mixture was stirred overnight at RT under atmosphere of hydrogen with a balloon. The solids were filtered out. The filtrate was concentrated under vacuum. This resulted in 13.8 g (80%) of the title compound as yellow oil. MS-ESI: 112 (M+1).

### Step 3: 1-Ethyl-1H-pyrazole-3-sulfonamide

To a stirred solution of 1-ethyl-1H-pyrazol-3-amine (3.4 g, 31 mmol,) in HCl (6 M, 20 mL) in a 100-mL round-bottom flask was added NaNO₂ (2.53 g, 37 mmol) in H₂O (5.0 mL) dropwise slowly at 0° C. over 25 min. The reaction solution was stirred for 40 min at 0° C., this solution was assigned as solution A. Then CuCl₂ (8.23 g, 61 mmol) was added to a 250-mL single necked round-bottom flask with AcOH (100 mL) as the solvent, SO₂ (g) was bubbled to the mixture with stirring at 0° C. for 20 min, this mixture was assigned as solution B. To the mixture B was added solution A dropwise with stirring at 0° C. The reaction mixture was stirred for additional 2 h at 0° C. The reaction mixture was diluted with 100 mL of H₂O. The mixture was extracted with 3×150 mL of DCM. The organic layers were dried over anhydrous Na₂SO₄ and concentrated under vacuum. The residue was dissolved in 30 mL of DCM and NH₃ (g) was bubbled for 15 min at 0° C. The reaction was stirred for 2 h at RT. The reaction mixture was concentrated under vacuum. The residue was eluted from silica gel with PE/EtOAc (3:1). This resulted in 1.75 g (33%) of the title compound as a yellow solid. MS-ESI: 174 (M−1).

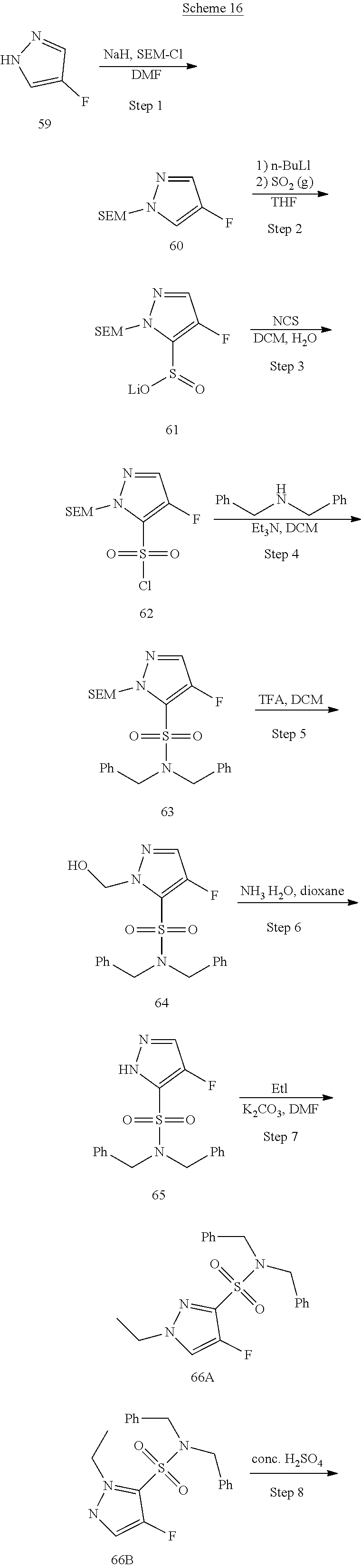

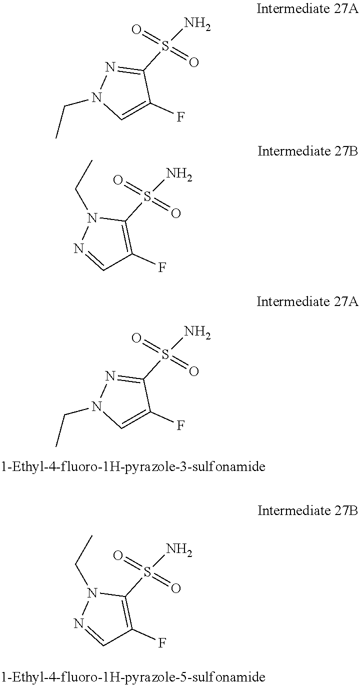

### Step 1: 4-Fluoro-1-((2-(trimethylsilyl)ethoxy)methyl)-1H-pyrazole

To a stirred solution of 4-fluoro-1H-pyrazole (5.0 g, 58 mmol) in DMF (53 mL) in a 250-mL 3-necked round-bottom flask under nitrogen was added NaH (60% wt. dispersion in mineral oil, 5.36 g, 134 mmol) in portions at 0° C. in an ice/water bath over 10 min. The resulting solution was stirred for 30 min at 10° C. To this was added SEM-Cl (22 g, 134 mmol) dropwise with stirring at 0° C. over 10 min. The resulting solution was stirred overnight at RT. The reaction was then quenched with 60 mL of water. The resulting solution was extracted with 60 mL of EtOAc. The organic layer was washed with 5×60 mL of sat. NaCl solution. The resulting filtrate was concentrated. The residue was eluted from silica gel column EtOAc/PE (1:100). This resulted in 13.7 g (crude) of the title compound as a light yellow liquid. MS-ESI: 217 (M+1).

### Step 2: Lithium 4-fluoro-1-((2-(trimethylsilyl)ethoxy)methyl)-1H-pyrazole-5-sulfinate

To a stirred solution of 4-fluoro-1-((2-(trimethylsilyl)ethoxy)methyl)-1H-pyrazole (13.7 g, 63 mmol) in THF (150 mL) in a 500-mL 3-necked round-bottom flask under nitrogen was added n-BuLi in hexane (2.5 M, 28 mL, 70 mmol) dropwise at −78° C. over 15 min. The resulting solution was stirred for 1 h at −78° C. Then to the mixture was introduced SO₂ (g) bubble for 20 min −78° C. The resulting solution was stirred for 1 h at RT. The resulting mixture was concentrated. This resulted in 20.4 g (crude) of the title compound as a white solid. MS-ESI: 279 (M−1).

### Step 3: 4-Fluoro-1-((2-(trimethylsilyl)ethoxy)methyl)-1H-pyrazole-5-sulfonyl chloride

To a stirred solution of lithium 4-fluoro-1-((2-(trimethylsilyl)ethoxy)methyl)-1H-pyrazole-5-sulfinate (20.4 g, crude from last step) in DCM (396 mL) and H₂O (198 mL) was added NCS (10 g, 78 mmol) in portions at 0° C. The resulting solution was stirred for 1 h at 10° C. The crude product was used directly without work-up.

### Step 4: N,N-dibenzyl-4-fluoro-1-((2-(trimethylsilyl)ethoxy)methyl)-1H-pyrazole-5-sulfonamide

To the stirred solution of 4-fluoro-1-((2-(trimethylsilyl)ethoxy)methyl)-1H-pyrazole-5-sulfonyl chloride in DCM (396 mL) and H₂O (198 mL) from last step was added Et₃N (8.85 g, 87 mmol) and dibenzylamine (17 g, 84 mmol) dropwise at 0° C. The resulting solution was stirred for 1 h at 8° C. The reaction was then quenched by the addition of 300 mL of water. The resulting solution was extracted with 3×300 mL of DCM. The organic layers were combined and washed with brine (300 mL) and dried over anhydrous Na₂SO₄ and concentrated under vacuum. The residue was eluted from silica gel with EtOAc/PE (1:19). This resulted in 22.5 g (81% over 4 steps) of the title compound as light yellow oil. MS-ESI: 476 (M+1).

### Step 5: N,N-dibenzyl-4-fluoro-1-(hydroxymethyl)-1H-pyrazole-5-sulfonamide

To a stirred solution of N,N-dibenzyl-4-fluoro-1-((2-(trimethylsilyl)ethoxy)methyl)-1H-pyrazole-5-sulfonamide (22.5 g, 47 mmol) in DCM (25 mL) in a 250-mL round-bottom flask was added TFA (25 mL). The resulting solution was stirred 16 h at RT. The resulting mixture was concentrated under vacuum. The residue was eluted from silica gel with EtOAc/PE (1:4). This resulted in 15 g (85%) of the title compound as yellow oil. MS-ESI: 376 (M+1).

### Step 6: N,N-dibenzyl-4-fluoro-1H-pyrazole-5-sulfonamide

To a stirred solution of N,N-dibenzyl-4-fluoro-1-(hydroxymethyl)-1H-pyrazole-5-sulfonamide (15 g, 40 mmol) in dioxane (50 mL) in a 500-mL round-bottom flask was added NH₃-H₂O (30% wt., 50 mL) dropwise at 0° C. The resulting solution was stirred for 3 h at RT. The resulting mixture was concentrated. The residue was eluted from silica gel with EtOAc/PE (1:1). This resulted in 12 g (87%) of the title compound as a white solid. MS-ESI: 346 (M+1).

### Step 7: N,N-dibenzyl-1-ethyl-4-fluoro-1H-pyrazole-3-sulfonamide and N,N-dibenzyl-1-ethyl-4-fluoro-1H-pyrazole-5-sulfonamide

To a stirred solution of N,N-dibenzyl-4-fluoro-1H-pyrazole-5-sulfonamide (1.1 g, 3.2 mmol) in DMF (20 mL) in a 100-mL 3-necked round-bottom flask under nitrogen was added K₂CO₃ (0.88 g, 6.4 mmol) in portions at RT and ethyl iodide (0.99 g, 6.4 mmol) dropwise at RT. The resulting solution was stirred for 3 h at 110° C. The reaction was then quenched by the addition of 20 mL of water. The resulting solution was extracted with 2×20 mL of EtOAc. The organic layers were dried over anhydrous Na₂SO₄ and concentrated under vacuum. The residue was eluted from silica gel with EtOAc/PE (3:17). This resulted in 218 mg (18%) of 66B followed by 764 mg (64%) of 66A both as a light yellow solid. MS-ESI: 374 (M+1).

### Step 8: 1-Ethyl-4-fluoro-1H-pyrazole-3-sulfonamide and 1-ethyl-4-fluoro-1H-pyrazole-5-sulfonamide

To a stirred solution of N,N-dibenzyl-1-ethyl-4-fluoro-1H-pyrazole-3-sulfonamide (764 mg, 2.0 mmol) in DCM (1.5 mL) in a 25-mL round-bottom flask was added H₂SO₄ (98% wt., 3.00 mL) dropwise at 0° C. The resulting solution was stirred for 1 h at RT. The reaction was then quenched by the addition of 5.0 mL of water/ice. The mixture was extracted with EtOAc (3×50 mL). The organic layer was dried over anhydrous Na₂SO₄ and concentrated under reduced vacuum. The residue was eluted from silica gel with DCM/MeOH (93:7). This resulted in 317 mg (80%) of Intermediate 27A as a white solid. MS-ESI: 194 (M+1). ¹H NMR (300 MHz, DMSO-d₆) δ 8.08 (d, J=4.7 Hz, 1H), 7.77 (s, 2H), 4.14 (q, J=7.3 Hz, 2H), 1.39 (t, J=7.3 Hz, 3H). Ar-H 8.08 (d, J=4.7 Hz, 1H) has correlations with CH₂ from Et at 4.14 (q, J=7.3 Hz, 2H) and CH₃ from Et at 1.39 (t, J=7.3 Hz, 3H) in NOESY.

Similar procedure used for converting compound 66A to Intermediate 27A shown in Scheme 16 was used to afford Intermediate 27B from compound 66B. MS-ESI: 194 (M+1). ¹H NMR (400 MHz, DMSO-d₆) δ 8.20 (s, 2H), 7.67 (d, J=4.5 Hz, 1H), 4.33 (q, J=7.2 Hz, 2H), 1.35 (t, J=7.2 Hz, 3H). NH₂ at 8.20 (s, 2H) has correlations with CH₂ from Et at 4.33 (q, J=7.2 Hz, 2H) and CH₃ from Et at 1.35 (t, J=7.2 Hz, 3H) in NOESY.

### Example 1 (Compound 8)

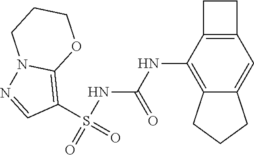 [SMILES: O=C(Nc1c2c(cc3c1CC3)CCC2)NS(=O)(=O)c1cnn2c1OCCC2]

### N-((2,4,5,6-tetrahydro-1H-cyclobuta[f]inden-3-yl)carbamoyl)-6,7-dihydro-5H-pyrazolo[5,1-b][1,3]oxazine-3-sulfonamide (Scheme 1)

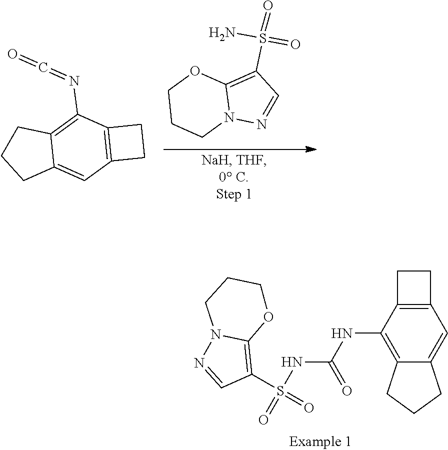

### Step 1: N-((2,4,5,6-tetrahydro-1H-cyclobuta[f]inden-3-yl)carbamoyl)-6,7-dihydro-5H-pyrazolo[5,1-b][1,3]oxazine-3-sulfonamide

Into a 100-mL round-bottom flask purged and maintained with an inert atmosphere of nitrogen, was placed a solution of 6,7-dihydro-5H-pyrazolo[5,1-b][1,3]oxazine-3-sulfonamide (102 mg, 0.5 mmol) in THF (10 mL). Then NaH (60% wt. oil dispersion, 24 mg, 1.0 mmol) was added in portions at 0° C. The resulting mixture was stirred for 5 min at room temperature, after which 3-isocyanato-2,4,5,6-tetrahydro-1H-cyclobuta[f]indene (90 mg, 0.5 mmol) was added. The resulting solution was stirred for 1 h at room temperature. The reaction was then quenched by the addition of 10 mL of water. The resulting solution was extracted with 3×50 mL of ethyl acetate. The combined organic layer was dried over anhydrous Na₂SO₄ and concentrated under vacuum. The residue was eluted from a silica gel column with dichloromethane/methanol (1:20). The product was further purified by Prep-HPLC with the following conditions: Column, XBridge Shield RP18 OBD Column, 19*250 mm, 10 um; mobile phase, Water (10 mM NH₄HCO₃) and ACN (19% Phase B up to 27% in 7 min); Detector, 210 nm UV. This resulted in 73.6 mg of the title compound as a white solid. (ES, m z): [M+1]⁺=389. ¹H-NMR (400 MHz, DMSO-d₆) S: 7.79 (s, 1H), 7.52 (s, 1H), 6.63 (s, 1H), 4.38 (t, J=5.2 Hz, 2H), 4.08 (t, J=6.1 Hz, 2H), 3.06 (t, J=4.4 Hz, 2H), 2.87 (t, J=4.3 Hz, 2H), 2.81 (t, J=7.4 Hz, 2H), 2.68 (t, J=7.3 Hz, 2H), 2.30-2.10 (m, 2H), 2.09-1.85 (m, 2H).

TABLE S5
Examples in the following table were prepared using similar conditions as
described in Example 1 and Scheme 1 from appropriate starting materials.

| | | | | Exact |
|---|---|---|---|---|
| Example | Compound | | | Mass |
| # | # | Structure | IUPAC Name | [M + H]⁺ |
| 2 | 9 | 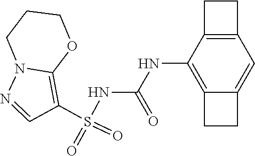 [SMILES: O=C(Nc1c2c(cc3c1CC3)CC2)NS(=O)(=O)c1cnn2c1OCCC2] | N-(tricyclo [6.2.0.03,6]deca- 1,3(6),7-trien-2- ylcarbamoyl)- 6,7-dihydro-5H- pyrazolo[5,1- b][1,3]oxazine-3- sulfonamide | 375 |
| 3 | 10 | 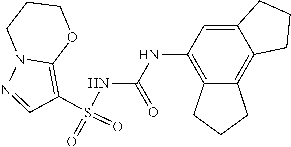 [SMILES: O=C(Nc1cc2c(c3c1CCC3)CCC2)NS(=O)(=O)c1cnn2c1OCCC2] | N-((1,2,3,6,7,8- hexahydro-as- indacen-4- yl)carbamoyl)- 6,7-dihydro-5H- pyrazolo[5,1- b][1,3]oxazine- 3-sulfonamide | 403 |

### Example 4 (Compound 12, Sodium Salt)

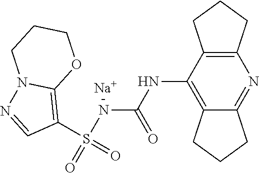 [SMILES: O=C(Nc1c2c(nc3c1CCC3)CCC2)NS(=O)(=O)c1cnn2c1OCCC2 ; [Na+]]

### Sodium ((6,7-dihydro-5H-pyrazolo[5,1-b][1,3]oxazin-3-yl)sulfonyl)((1,2,3,5,6,7-hexahydrodicyclopenta[b,e]pyridin-8-yl)carbamoyl)amide (Scheme 2)

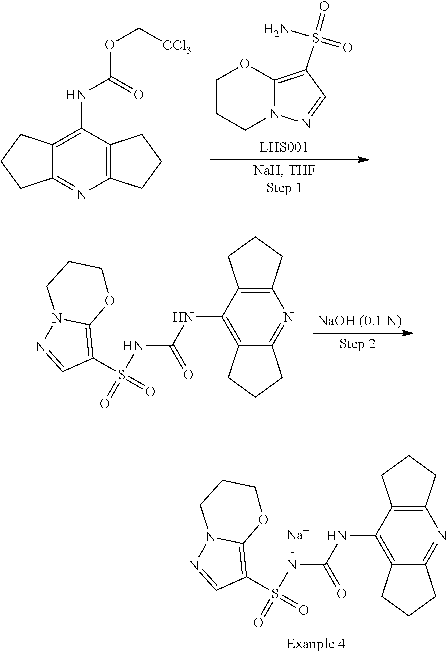

### Step 1: N-((1,2,3,5,6,7-hexahydrodicyclopenta[b,e]pyridin-8-yl)carbamoyl)-6,7-dihydro-5H-pyrazolo[5,1-b][1,3]oxazine-3-sulfonamide

Into a 25-mL round-bottom flask purged and maintained with an inert of atmosphere of nitrogen, was placed 6,7-dihydro-5H-pyrazolo[5,1-b][1,3]oxazine-3-sulfonamide (100 mg, 0.49 mmol) in THF (8.0 mL). NaH (60% wt. oil dispersion, 88.6 mg, 1.48 mmol) was added at 0° C. The solution was stirred for 15 min. Then 2,2,2-trichloroethyl (1,2,3,5,6,7-hexahydrodicyclopenta[b,e]pyridin-8-yl)carbamate (172 mg, 0.49 mmol) was added into the above solution at 0° C. The resulting solution was stirred for 4 h at room temperature. The reaction was then quenched by the addition of 5 mL of water. The resulting mixture was concentrated under vacuum. The residue was applied onto a silica gel column with dichloromethane/methanol (5:1). The crude product (160 mg) was further purified by Prep-HPLC with the following conditions (Column: XBridge Prep C₁₈ OBD Column 19×150 mm 5 um; Mobile Phase A: Water (10 mM NH₄HCO₃+0.1% NH₃.H₂O), Mobile Phase B: ACN; Flow rate: 25 mL/min; Gradient: 5% B to 11% B in 7 min; 254/210 nm; Rt: 5.77 min). This resulted in the title compound (22 mg, 10.5%) as a white solid. MS-ESI: 430 (M+1).

### Step 2: Sodium ((6,7-dihydro-5H-pyrazolo[5,1-b][1,3]oxazin-3-yl)sulfonyl)((1,2,3,5,6,7-hexahydrodicyclopenta[b,e]pyridin-8-yl)carbamoyl)amide

N-((1,2,3,5,6,7-hexahydrodicyclopenta[b,e]pyridin-8-yl)carbamoyl)-6,7-dihydro-5H-pyrazolo[5,1-b][1,3]oxazine-3-sulfonamide (22 mg, 0.06 mmol) obtained in the last step was dissolved in NaOH (0.1N) (0.44 mL, 0.04 mmol), after which the solution was washed with DCM (1.0 mL). The aqueous phase was collected and filtered, then lyophilized to obtain 13.9 mg (59.90%) of title compound as white solid (mono sodium salt). MS-ESL: 418 (M+1), 416 (M−1). ¹H-NMR (400 MHz, D₂O) δ 7.55 (s, 1H), 4.36-4.29 (m, 2H), 4.03 (t, J=6.1 Hz, 2H), 3.00-2.50 (m, 8H), 2.17-2.10 (m, 2H), 2.10-1.90 (m, 4H).

TABLE S6
Examples in the following table were prepared using similar conditions as
described in Example 4 and Scheme 2 from appropriate starting materials.

| | Com- | | | Exact |
|---|---|---|---|---|
| Example | pound | | | Mass |
| # | # | Structure | IUPAC Name | [M + H]⁺ |
| 5 | 13 (sodium salt) | 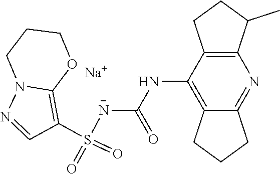 [SMILES: CC1CCc2c1nc1c(c2NC(=O)[N-]S(=O)(=O)c2cnn3c2OCCC3)CCC1 ; [Na+]] | Sodium ((6,7-dihydro-5H- pyrazolo[5,1-b][1,3]oxazin-3- yl)sulfonyl)((3-methyl-1,2,3,5,6,7- hexahydrodicyclopenta[b,e]pyridin- 8-yl)carbamoyl)amide | 418 |
| 6 | 15 (sodium salt) | 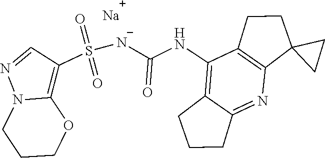 [SMILES: [Na+] ; O=C([N-]S(=O)(=O)c1cnn2c1OCCC2)Nc1c2c(nc3c1CCC31CC1)CCC2] | Sodium ((6,7-dihydro-5H- pyrazolo[5,1-b][1,3]oxazin-3- yl)sulfonyl)((1′,5′,6′,7′-tetrahydro- 2′H-spiro[cyclopropane-1,3′- dicyclopenta[b,e]pyridin]-8′- yl)carbamoyl)amide | 430 |
| 7 | 16 (sodium salt) | 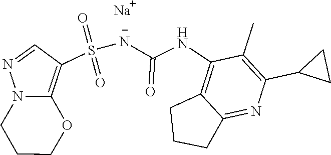 [SMILES: [Na+] ; Cc1c(C2CC2)nc2c(c1NC(=O)[N-]S(=O)(=O)c1cnn3c1OCCC3)CCC2] | Sodium ((2-cyclopropyl-3-methyl- 6,7-dihydro-5H- cyclopenta[b]pyridin-4- yl)carbamoyl)((6,7-dihydro-5H- pyrazolo[5,1-b][1,3]oxazin-3- yl)sulfonyl)amide | 418 |

TABLE S7
Examples in the following table were prepared using similar conditions as
described in Example 4 and Scheme 2 from appropriate starting materials.

| | Final | | | Exact |
|---|---|---|---|---|
| Example | Target | | | Mass |
| # | # | Structure | IUPAC Name | [M + H]⁺ |
| 8 | 14 | 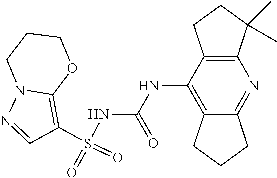 [SMILES: CC1(C)CCc2c1nc1c(c2NC(=O)NS(=O)(=O)c2cnn3c2OCCC3)CCC1] | N-((3,3-dimethyl- 1,2,3,5,6,7- hexahydro- dicyclopenta [b,e]pyridin-8- yl)carbamoyl)-6,7- dihydro-5H- pyrazolo[5,1- b][1,3]oxazine-3- sulfonamide | 432 |

### Example 10 (Compound 401)

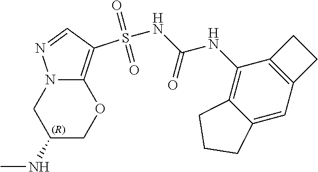

### (R)-6-(methylamino)-N-((2,4,5,6-tetrahydro-1H-cyclobuta[f]inden-3-yl)carbamoyl)-6,7-dihydro-5H-pyrazolo[5,1-b][1,3]oxazine-3-sulfonamide (Scheme I)

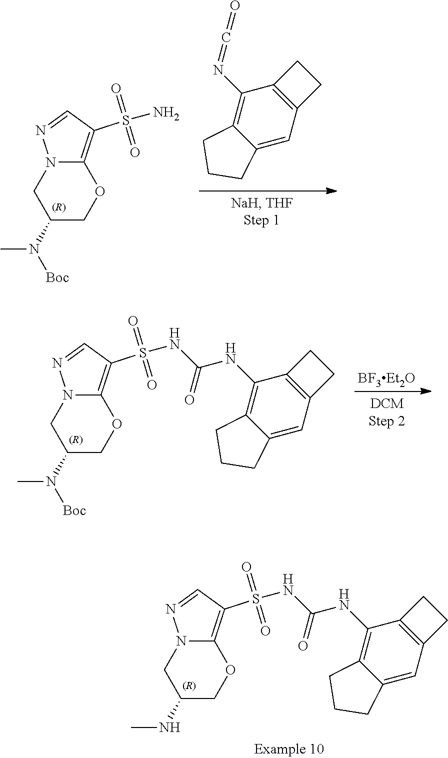

### Step 1: Tert-butyl (R)-methyl(3-(N-((2,4,5,6-tetrahydro-1H-cyclobuta[f]inden-3-yl)carbamoyl) sulfamoyl)-6,7-dihydro-5H-pyrazolo[5,1-b][1,3]oxazin-6-yl)carbamate

To a stirred solution of tert-butyl (R)-methyl(3-sulfamoyl-6,7-dihydro-5H-pyrazolo[5,1-b][1,3]oxazin-6-yl) carbamate (66.4 mg, 0.20 mmol) in THF (10 mL) in a 100-mL round-bottom flask under nitrogen was added NaH (60% wt. dispersion in mineral oil, 24 mg, 0.60 mmol) at 0° C. The resulting solution was stirred for 10 min at RT. To the solution was added 3-isocyanato-2,4,5,6-tetrahydro-1H-cyclobuta[f]indene (37 mg, 0.20 mmol) in THF (1.0 mL) dropwise at RT. The resulting solution was stirred for 1 h at RT. The reaction was then quenched by the addition of 5 mL of MeOH. The resulting mixture was concentrated. The residue was eluted from silica gel with DCM/MeOH (10:1). This resulted in 98 mg (95%) of the title compound as a light brown solid. MS-ESI: 518 (M+1).

### Step 2: (R)-6-(methylamino)-N-((2,4,5,6-tetrahydro-1H-cyclobuta[f]inden-3-yl)carbamoyl)-6,7-dihydro-5H-pyrazolo[5,1-b][1,3]oxazine-3-sulfonamide

TABLE 12
Examples in the following table were prepared using similar conditions as
described in Example 1 and Scheme 1 from appropriate starting materials.

| | | | | Exact |
|---|---|---|---|---|
| Example | Compound | | | Mass |
| # | Number | Structure | IUPAC Name | [M + H]⁺ |
| 11 | 403 | 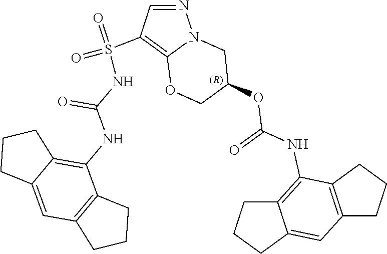 | (R)-3-(N-((1,2,3,5,6,7- hexahydro-s-indacen-4- yl)carbamoyl)sulfamoyl)- 6,7-dihydro-5H- pyrazolo[5,1- b][1,3]oxazin-6-yl (1,2,3,5,6,7-hexahydro-s- indacen-4-yl)carbamate | 618 |
| 12 | 301 | 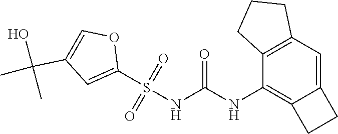 [SMILES: CC(C)(O)c1coc(S(=O)(=O)NC(=O)Nc2c3c(cc4c2CC4)CCC3)c1] | 4-(2-Hydroxypropan-2- yl)-N-((2,4,5,6- tetrahydro-1H- cyclobuta[f]inden-3- yl)carbamoyl)furan-2- sulfonamide | 391 |
| 13 | 402 | 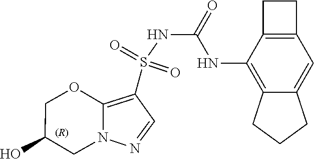 | (R)-6-hydroxy-N- ((2,4,5,6-tetrahydro-1H- cyclobuta[f]inden-3- yl)carbamoyl)-6,7- dihydro-5H- pyrazolo[5,1- b][1,3]oxazine-3- sulfonamide | 405 |
| 14 | 302 | 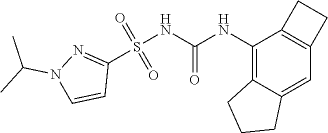 [SMILES: CC(C)n1ccc(S(=O)(=O)NC(=O)Nc2c3c(cc4c2CC4)CCC3)n1] | 1-Isopropyl-N-((2,4,5,6- tetrahydro-1H- cyclobuta[f]inden-3- yl)carbamoyl)-1H- pyrazole-3-sulfonamide | 375 |

To a stirred solution of tert-butyl (R)-methyl(3-(N-((2,4,5,6-tetrahydro-1H-cyclobuta[f]inden-3-yl)-carbamoyl)sulfamoyl)-6,7-dihydro-5H-pyrazolo[5,1-b][1,3]oxazin-6-yl)carbamate (90 mg, 0.17 mmol) in DCM (10 mL) in a 100-mL round-bottom flask was added BF₃.Et₂O (47% wt., 25 mg, 0.17 mmol) at RT. The resulting solution was stirred for 1 h at RT. The resulting mixture was concentrated. The residue was eluted from silica gel with DCM/MeOH (10:1). The crude product was purified by Prep-HPLC with the following conditions: XSelect CSH Prep C18 OBD Column, 5 um, 19*150 mm; Mobile Phase A: water (10 mM NH₄HCO₃+0.1% NH₃.H₂O), Mobile Phase B: ACN; Flow rate: 25 mL/min; Gradient:17% B to 27% B over 7 min; UV 210/254 nm; Rt1: 4.50 min. This resulted in 12.6 mg (17%) of the title compound as an off-white solid. MS-ESI: 418 (M+1). ¹H NMR (400 MHz, DMSO-d₆) δ 7.91 (br, s, 1H), 7.60 (s, 1H), 6.67 (s, 1H), 4.39-4.28 (m, 2H), 4.23-4.19 (m, 1H), 3.96-3.92 (m, 1H), 3.21-3.13 (m, 1H), 3.09-3.01 (m, 2H), 2.91-2.85 (m, 2H), 2.84-2.78 (m, 2H), 2.71-2.62 (m, 2H), 2.31 (s, 3H), 2.03-1.89 (m, 2H).

TABLE 13
Examples in the following table were prepared using similar conditions as
described in Example 4 and Scheme 2 from appropriate starting materials.

| | | | | Exact |
|---|---|---|---|---|
| Example | Compound | | | Mass |
| # | Number | Structure | IUPAC Name | [M + H]⁺ |
| 15 | 404 | 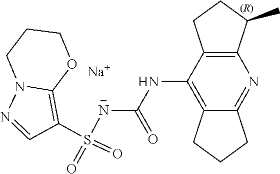 | Sodium (R)-((6,7- dihydro-5H- pyrazolo[5,1- b][1,3]oxazin-3- yl)sulfonyl)((3-methyl- 1,2,3,5,6,7- hexahydrodicyclopenta [b,e]pyridin-8- yl)carbamoyl)amide | 440 |

TABLE 14
Examples in the following table were prepared using similar conditions as
described in Example 1 and Scheme 1 from appropriate starting materials.

| | | | Exact |
|---|---|---|---|
| Example | | | Mass |
| # | Structure | IUPAC Name | [M + H]⁺ |
| 16 | 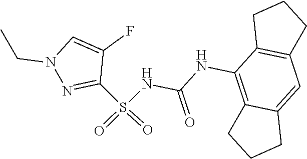 [SMILES: CCn1cc(F)c(S(=O)(=O)NC(=O)Nc2c3c(cc4c2CCC4)CCC3)n1] | 1-Ethyl-4-fluoro- N-((1,2,3,5,6,7- hexahydro-s- indacen-4- yl)carbamoyl)- 1H-pyrazole-3- sulfonamide | 393 |
| 17 | 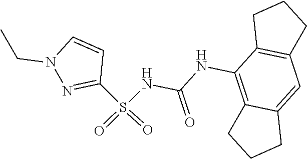 [SMILES: CCn1ccc(S(=O)(=O)NC(=O)Nc2c3c(cc4c2CCC4)CCC3)n1] | 1-Ethyl-N- ((1,2,3,5,6,7- hexahydro-s- indacen-4- yl)carbamoyl)- 1H-pyrazole-3- sulfonamide | 375 |
| 18 | 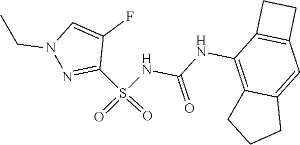 [SMILES: CCn1cc(F)c(S(=O)(=O)NC(=O)Nc2c3c(cc4c2CC4)CCC3)n1] | 1-Ethyl-4-fluoro- N-((2,4,5,6- tetrahydro-1H- cyclobuta[f]inden- 3-yl)carbamoyl)- 1H-pyrazole-3- sulfonamide | 379 |
| 19 | 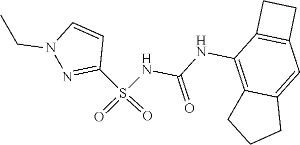 [SMILES: CCn1ccc(S(=O)(=O)NC(=O)Nc2c3c(cc4c2CC4)CCC3)n1] | 1-Ethyl-N- ((2,4,5,6- tetrahydro-1H- cyclobuta[f]inden- 3-yl)carbamoyl)- 1H-pyrazole-3- sulfonamide | 361 |

In one embodiment, provided herein is a pharmaceutical composition comprising a specific compound of the invention defined herein (e.g. any one of examples 1 to 19, or tables 1A to 1C, 2, 3 or 4), and an anti-TNFα agent disclosed herein. Preferably wherein the anti-TNFαagent is Infliximab, Etanercept, Certolizumab pegol, Golimumab or Adalimumab, more preferably wherein the anti-TNFα agent is Adalimumab.

In one embodiment, provided herein is a pharmaceutical combination comprising a specific compound of the invention defined herein (e.g. any one of examples 1 to 19, or tables 1A to 1C, 2, 3 or 4), and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

The following protocol is suitable for testing the activity of the compounds disclosed herein.

Procedure 1: IL-1β production in PMA-differentiated THP-1 cells stimulated with Gramicidin.

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 μg/ml)), and maintained in log phase prior to experimental setup. Prior to the experiment THP-1 were treated with PMA (Phorbol 12-myristate 13-acetate) (20 ng/ml) for 16-18 hours. Compounds were dissolved in dimethyl sulfoxide (DMSO) to generate a 30 mM stock. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 μg/ml). The cells were plated in a 384-well plate at a density of 50,000 cells/well (final assay volume 50 μl). Compounds were first dissolved in assay medium to obtain a 5× top concentration of 500 μM. 10 step dilutions (1:3) were then undertaken in assay medium containing 1.67% DMSO. 5× compound solutions were added to the culture medium to achieve desired final concentration (e.g. 100, 33, 11, 3.7, 1.2, 0.41, 0.14, 0.046, 0.015, 0.0051, 0.0017 μM). Final DMSO concentration was at 0.37%. Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

### Procedure 2

### 1. Experimental Procedure

1.1 Cell Culture
1) Culture THP-1 cells in the complete RPMI-1640 medium with 10% FBS at 37° C., 5% CO₂.
2) Passage the cells every 3 days by inoculating 3×10⁵ cells per ml.

1.2 Compound Preparation
Prepare the 3-fold serial dilution of the compounds with DMSO in a 384-well LDV Microplate using TECAN EVO system to generate the compound source plate with 10 concentrations. Top concentration is 30 mM.

1.3 Cell Preparation
1) Centrifuge THP-1 cells at 350 g for 5 min.
2) Re-suspend cells with complete RMPI-1640 medium, and count cells.
3) Seed cells in T225 flask, about 2.5×10⁷ per flask, treat cells with 20 ng/ml PMA (final DMSO concentration<1%).
4) Incubate overnight.

1.4 THP-1 Stimulation
1) Wash adherent THP-1 cells with PBS, and detach cells with 4 ml trypsin for T225 flask.
2) Centrifuge cells at 350 g for 5 min, re-suspend cells with RPMI-1640 containing 2% FBS and count cells with trypan blue.
3) Transfer 50 nl/well the serial dilution of test compound to 384-well plate by Echo; For the high control and first point of CRID3 (MCC950), transfer 165 nl, then backfill to make the DMSO concentration is consistent in all wells, the plate layout is as below.
4) Seed 50k cells in 40 ul RPMI-1640 with 2% FBS per well in 384-well plate.
5) Incubate for 1 h at 37′C, 5% CO₂.
6) Prepare 5× gramicidin, add 10 μl per well, the final concentration is 5 μM, incubate for 2 hrs at 37° C., 5% CO₂.
7) Centrifuge at 350 g for 1 min.
8) Pipet 16 μl supernatant by apricot, and transfer into white 384 proxiplate. FIG. 3 depicts the layout of the plates: HC: 100 μM CRID3 (MCC950)+5 μM gramicidin LC: 5 μM Gramicidin.

1.5 IL-1β detection
1) Homogenize the 5× diluent #5 with a vortex and add 1 volume of stock solution in 4 volumes of distilled water.
2) Thaw 20× stock solution of anti-IL1β-Cryptate-antibody and anti-IL1β XL-antibody. Dilute these two antibodies to 1× with detection buffer #3.
3) Pre-mix the two ready-to-use antibody solutions just prior to use.
4) Dispense 4 ul of pre-mixed Anti-IL1β antibodies working solution into all wells.
5) Seal the plate and incubate overnight at 4° C.
6) Read the cell plate using EnVison and plot Readout vs. the test compound concentration to calculate the IC₅₀.

### 2. Data Analysis:

1. IC₅₀ of compounds can be calculated using the following formulas

% inhibition=100−100×[HCₐᵥₑ−Readout/(HCₐᵥₑ−LCₐᵥₑ)] Formula for IC₅₀
2. Fit the normalized data in a dose-response manner using XLfit, and calculate the compound concentration.
   Tables 2 and 3 show the biological activity of compounds in hTHP-1 assay containing 2% fetal bovine serum and Table 4 shows the biological activity of compounds in hTHP-1 assay containing 10% fetal bovine serum: <0.008 μM=“++++++”; >0.008 and <0.04 μM=“+++++”; ≥0.04 and <0.2 μM=“++++”; ≥0.2 and <1 μM=“+++”; ≥1 and <5 μM=“++”; >5 and ≤30 μM

TABLE 2
Average IC₅₀ of compounds in hTHP-1 assay

| Compound | hTHP-1 |
|---|---|
| Number | IC₅₀ |
| 8 | +++ |
| 9 | ++++ |
| 10 | ++ |
| 12 | +++ |
| 13 | +++ |
| 14 | ++ |
| 15 | ++ |
| 16 | ++ |
| 401 | ++++++ |
| 402 | ++++ |
| 403 | + |
| 404 | +++ |

TABLE 3
Average IC₅₀ of compounds in hTHP-1 assay

| Compound | hTHP-1 IC₅₀ |
|---|---|
| 301 | ++++ |
| 302 | ++ |
| 303 | ++ |

TABLE 4
Average IC₅₀ of compounds in hTHP-1
assay

| | hTHP-1 IC₅₀ in |
|---|---|
| Example # | 10% FBS |
| 16 | +++ |
| 17 | ++ |
| 18 | +++ |
| 19 | +++ |

### Study Example 1

The CARD8 gene is located within the inflammatory bowel disease (IBD) 6 linkage region on chromosome 19. CARD8 interacts with NLRP3, and Apoptosis-associated Speck-like protein to form a caspase-1 activating complex termed the NLRP3 inflammasome. The NLRP3 inflammasome mediates the production and secretion of interleukin-1β, by processing pro-IL-1β into mature secreted IL-1β. In addition to its role in the inflammasome, CARD8 is also a potent inhibitor of nuclear factor NF-κB. NF-κB activation is essential for the production of pro-IL-1δ. Since over-production of IL-1β and dyregulation of NF-κB are hallmarks of Crohn's disease, CARD8 is herein considered to be a risk gene for inflammatory bowel disease. A significant association of CARD8 with Crohn's disease was detected in two British studies with a risk effect for the minor allele of the non-synonymous single-nucleotide polymorphism (SNP) of a C allele at rs2043211. This SNP introduces a premature stop codon, resulting in the expression of a severely truncated protein. This variant CARD8 protein is unable to suppress NF-κB activity, leading to constitutive production of pro-IL-1β, which is a substrate for the NLRP3 inflammasome. It is believed that a gain-of-function mutation in an NLRP3 gene (e.g., any of the gain-of-function mutations described herein, e.g., any of the gain-of-function mutations in an NLRP3 gene described herein) combined with a loss-of-function mutation in a CARD8 gene (e.g., a C allele at rs2043211) results in the development of diseases related to increased NLRP3 inflammasome expression and/or activity. Patients having, e.g., a gain-of-function mutation in an NLRP3 gene and/or a loss-of-function mutation in a CARD8 gene are predicted to show improved therapeutic response to treatment with an NLRP3 antagonist.

A study is designed to determine: whether NLRP3 antagonists inhibit inflammasome function and inflammatory activity in cells and biopsy specimens from patients with Crohn's disease or ulcerative colitis; and whether the specific genetic variants identify patients with Crohn's disease or ulcerative colitis who are most likely to respond to treatment with an NLRP3 antagonist.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if baseline (no ex vivo treatment) RNA levels of NLRP3, ASC, and IL-1β are greater in biopsy samples from patients with anti-TNFα agent resistance status; and stratify the results according to presence of specific genetic mutations in genes encoding ATG16L1, NLRP3, and CARD8 (e.g., any of the mutations in the ATG16L1 gene, NLRP3 gene, and CARD8 gene described herein).

Methods
Evaluation of baseline expression of NLRP3 RNA and quantify inhibition of inflammasome activity by an NLRP3 antagonist in biopsies of disease tissue from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.
Sequence patient DNA to detect specific genetic mutations in the ATG16L1 gene, NLRP3 gene, and CARD8 gene (e.g., any of the exemplary mutations in these genes described herein) and then stratify the results of functional assays according to the presence of these genetic mutations.

Experimental Design
Human subjects and tissue:
   Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.
Ex vivo Treatment Model:
   Organ or LPMC culture as determined appropriate
Endpoints to be measured:
   Before ex vivo treatment—NLRP3 RNA level
   After ex vivo treatment—inflammasome activity (either processed IL-1β, processed caspase-1, or secreted IL-1β); RNA for inflammatory cytokines (Nanostring); viable T cell number and/or T cell apoptosis.
Data Analysis Plan:
   Determine if NLRP3 antagonist treatment decreases processed IL-1β, processed caspase-1 or secreted IL-1β, and inflammatory cytokine RNA levels.
   Stratify response data according to treatment status at biopsy and the presence of genetic mutations in the NLRP3 gene, CARD8 gene, and ATG16L1 gene (e.g., any of the exemplary genetic mutations of these genes described herein).

### Study Example 2. Treatment of Anti-TNFα Resistant Patients with NLRP3 Antagonists

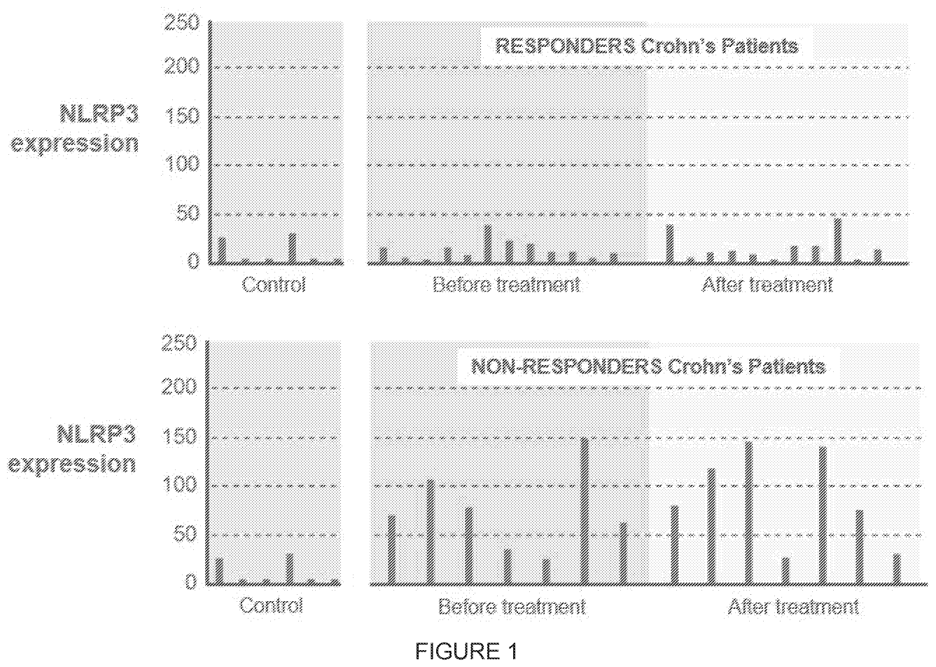

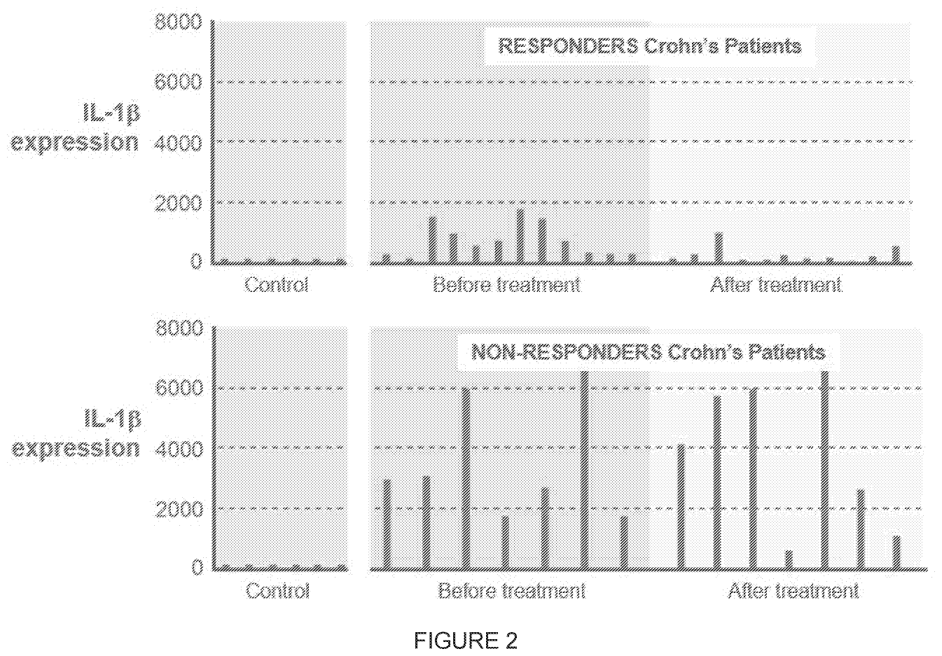

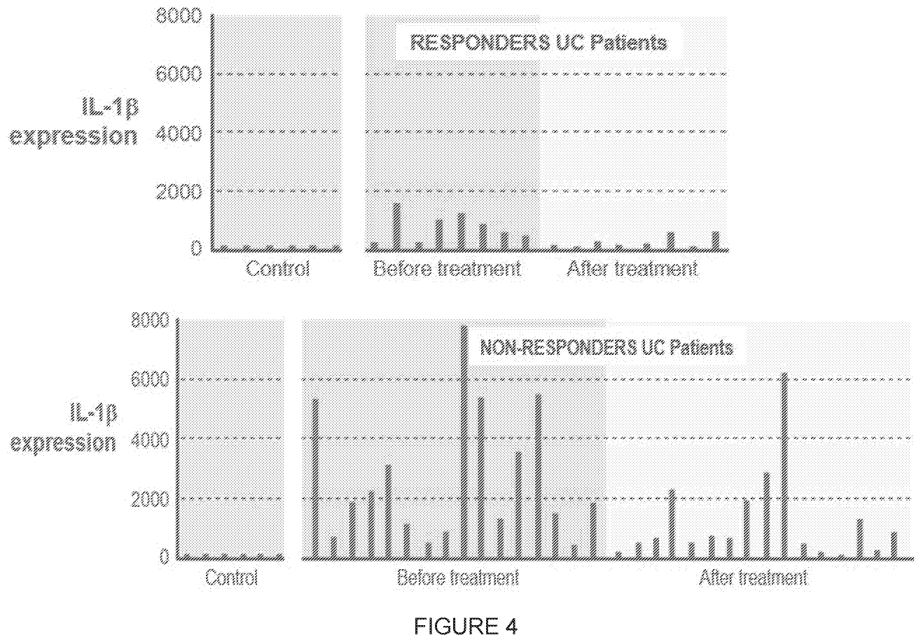

PLoS One 2009 Nov. 24; 4(11):e7984, describes that mucosal biopsies were obtained at endoscopy in actively inflamed mucosa from patients with Ulcerative Colitis, refractory to corticosteroids and/or immunosuppression, before and 4-6 weeks after their first infliximab (an anti-TNFα agent) infusion and in normal mucosa from control patients. The patients in this study were classified for response to infliximab based on endoscopic and histologic findings at 4-6 weeks after first infliximab treatment as responder or non-responder. Transcriptomic RNA expression levels of these biopsies were accessed by the inventors of the invention disclosed herein from GSE 16879, the publically available Gene Expression Omnibus (https://www.nebi.nlm.nih.gov/geo/geo2r/?acc=GSE16879). Expression levels of RNA encoding NLRP3 and IL-1β were determined using GEO2R (a tool available on the same website), based on probe sets 207075_at and 205067_at, respectively. It was surprisingly found that in Crohn's disease patients that are non-responsive to the infliximab (an anti-TNFα agent) have higher expression of NLRP3 and IL-1β RNA than responsive patients (FIGS. 1 and 2). Similar surprising results of higher expression of NLRP3 and IL-1β RNA in UC patients that are non-responsive to infliximab (an anti-TNFα agent) compared to infliximab (an anti-TNFα agent) responsive patients (FIGS. 3 and 4) were found.

Said higher levels of NLRP3 and IL-1β RNA expression levels in anti-TNFα agent non-responders, is hypothesised herein to lead to NLRP3 activation which in turns leads of release of IL-1β that induces IL-23 production, leading to said resistance to anti-TNFα agents. Therefore, treatment of Crohn's and UC anti-TNFα non-responders with an NLRP3 antagonist would prevent this cascade, and thus prevent development of non-responsiveness to anti-TNFα agents. Indeed, resistance to anti-TNFα agents is common in other inflammatory or autoimmune diseases. Therefore, use of an NLRP3 antagonist for the treatment of inflammatory or autoimmune diseases will block the mechanism leading to non-responsiveness to anti-TNFα agents. Consequently, use of NLRP3 antagonists will increase the sensitivity of patients with inflammatory or autoimmune diseases to anti-TNFα agents, resulting in a reduced dose of anti-TNFα agents for the treatment of these diseases. Therefore, a combination of an NLRP3 antagonist and an anti-TNFα agent can be used in the treatment of diseases wherein TNFα is overexpressed, such as inflammatory or autoimmune diseases, to avoid such non-responsive development of patients to anti-TNFα agents. Preferably, this combination treatment can be used in the treatment of IBD, for example Crohn's disease and UC.

Further, use of NLRP3 antagonists offers an alternative to anti-TNFα agents for the treatment of diseases wherein TNFα is overexpressed. Therefore, NLRP3 antagonists offers an alternative to anti-TNFα agents inflammatory or autoimmune diseases, such as IBD (e.g. Crohn's disease and UC).

Systemtic anti-TNFα agents are also known to increase the risk of infection. Gut restricted NLRP3 antagonists, however, offers a gut targeted treatment (i.e. non-systemic treatment), preventing such infections. Therefore, treatment of TNFα gut diseases, such as IBD (i.e. Crohn's disease and UC), with gut restricted NLRP3 antagonists has the additional advantage of reducing the risk of infection compared to anti-TNFα agents.

Proposed Experiment:

Determine the expression of NLRP3 and caspase-1 in LPMCs and epithelial cells in patients with non-active disease, in patients with active disease, in patients with active disease resistant to corticosteroids, patients with active disease resistant to TNF-blocking agents. The expression of NLRP3 and caspase-1 in LPMCs and epithelial cells will be analyzed by RNAScope technology. The expression of active NLRP3 signature genes will be analyzed by Nanostring technology. A pilot analysis to determine feasibility will be performed with 5 samples from control, 5 samples from active CD lesions, and 5 samples from active UC lesions.

### Study Example 3

It is presented that NLRP3 antagonists reverse resistance to anti-TNF induced T cell depletion/apoptosis in biopsy samples from IBD patients whose disease is clinically considered resistant or unresponsive to anti-TNF therapy.

A study is designed to determine: whether NLRP3 antagonists inhibit inflammasome function and inflammatory activity in cells and biopsy specimens from patients with Crohn's disease or ulcerative colitis; and whether an NLRP3 antagonist will synergize with anti-TNFα therapy in patients with Crohn's disease or ulcerative colitis.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if an NLRP3 antagonist in the absence of co-treatment with anti-TNFα antibody induces T cell depletion in Crohn's disease and ulcerative colitis biopsy samples; and determine if baseline (no ex vivo treatment) RNA levels of NLRP3, ASC, and IL-1β are greater in biopsy samples from patients with anti-TNFα agent resistance status.

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if there is synergy between an NLRP3 antagonist and anti-TNF antibody with respect to effects on T cell depletion/apoptosis in biopsies of disease from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.

### Experimental Design

Human subjects and tissue:
   Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.
Ex vivo Treatment Model:
   Organ or LPMC culture as determined appropriate
Ex vivo Treatments:
   NLRP3 antagonist (2 concentrations), negative control (vehicle), positive control (caspase-1 inhibitor) each in the presence or absence of anti-TNF antibody at a concentration appropriate to distinguish differences in the T cell apoptotic between biopsies from anti-TNF resistant and anti-TNF-sensitive Crohn's disease patients. Each treatment condition is evaluated in a minimum in duplicate samples.
Endpoints to be measured:
   Before ex vivo treatment—NLRP3 RNA level
   After ex vivo treatment—inflammasome activity (either processed IL-1β, processed caspase-1, or secreted IL-1β); RNA for inflammatory cytokines (Nanostring); viable T cell number and/or T cell apoptosis.
Data Analysis Plan:
   Determine if NLRP3 antagonist co-treatment increases T cell apoptosis/deletion in response to anti-TNF.
   Determine if the level of NLRP3 RNA expression is greater in TNF-resistant Crohn's disease and ulcerative colitis samples compared to anti-TNF treatment-naïve samples.
Determine if NLRP3 antagonist treatment decreases processed IL-1β, processed caspase-1 or secreted IL-1β, and inflammatory cytokine RNA levels.

### Biological Assay—Nigericin-Stimulated IL-1β Secretion Assay in THP-1 Cells

Monocytic THP-1 cells (ATCC: TIB-202) were maintained according to providers' instructions in RPMI media (RPMI/Hepes+10% fetal bovine serum+Sodium Pyruvate+0.05 mM Beta-mercaptoethanol (1000× stock)+Pen-Strep). Cells were differentiated in bulk with 0.5 μM phorbol 12-myristate 13-acetate (PMA; Sigma #P8139) for 3 hours, media was exchanged, and cells were plated at 50,000 cells per well in a 384-well flat-bottom cell culture plates (Greiner, #781986), and allowed to differentiate overnight. Compound in a 1:3.16 serial dilution series in DMSO was added 1:100 to the cells and incubated for 1 hour. The NLRP3 inflammasome was activated with the addition of 15 μM (final concentration) Nigericin (Enzo Life Sciences, #BML-CA421-0005), and cells were incubated for 3 hours. 10 μL supernatant was removed, and IL-1 (3 levels were monitored using an HTRF assay (CisBio, #62IL1PEC) according to manufacturers' instructions. Viability and pyroptosis was monitored with the addition of PrestoBlue cell viability reagent (Life Technologies, #A13261) directly to the cell culture plate.

A number of embodiments of the invention have been described. Nevertheless, it will be understood that various modifications may be made without departing from the spirit and scope of the invention. Accordingly, other embodiments are within the scope of the following claims.

What is claimed is:

1. A compound of Formula AA 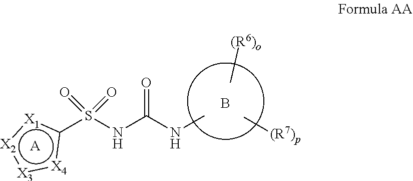
wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
when X⁴ is —X⁵—X⁶, then: X¹ is N or CR¹, X² is N or CR², and X³ is N or CR³;
when X⁴ is other than —X⁵—X⁶—, then 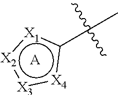
comprises at least one of CR¹, CR², CR³, and CR⁴;
when X⁴ is —X⁵—X⁶—, then 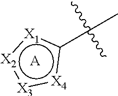
comprises at least two of CR¹, CR², CR³, CR⁵, and CR⁶;
when X⁴ is other than —X⁵—X⁶—, then 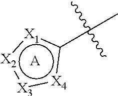
comprises at least one of CR¹, CR², CR³, and CR⁴;
when X⁴ is —X⁵—X⁶—, then 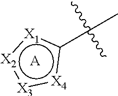
comprises at least two of CR¹, CR², CR³, CR⁵, and CR⁶;
from two to four of R¹, R², R³, and R⁴ are present or from two to five of R¹, R², R³, R⁵, and R⁶ are present; and
wherein at least two of the two to four R¹, R², R³, and R⁴ or at least two of the two to five R¹, R², R³, R⁵, and R⁶ are on adjacent atoms, and taken together with the atoms connecting them, independently form a ring selected from the group consisting of:
(i) a monocyclic or bicyclic C₄-C₁₂ cycloalkyl ring optionally substituted with one or more R²⁰,
(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰,
(iii) a monocyclic or bicyclic C₆-C₁₀ aryl ring optionally substituted with one or more R²⁰, and
(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heteroaryl ring is optionally substituted with one or more R²⁰;
R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹; or
at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;
R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
wherein any of R¹, R², R³, R⁴, R⁵, and R⁶ that are not taken together with the atoms connecting them to form a ring, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;
R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;
R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
B is a 5-10-membered heteroaryl or C₆-C₁₀ aryl ring;
o=1 or 2;
p=0, 1, 2, or 3;
R⁶ and R⁷ are each independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
provided that when B is 5-10-membered heteroaryl including from 2-3 ring nitrogen atoms, at least one R⁶ is attached to B at a position ortho to the —HNC(═O)NHS(O)₂— moiety of Formula AA;
when B is 2-pyridyl, pyrimidin-6-yl, or pyrimidin-4-yl, B is not substituted with a cyano group at a position ortho to the —HNC(═O)NHS(O)₂— moiety of Formula AA;
and
with the proviso that the compound of Formula AA is not a compound selected from the group consisting of: 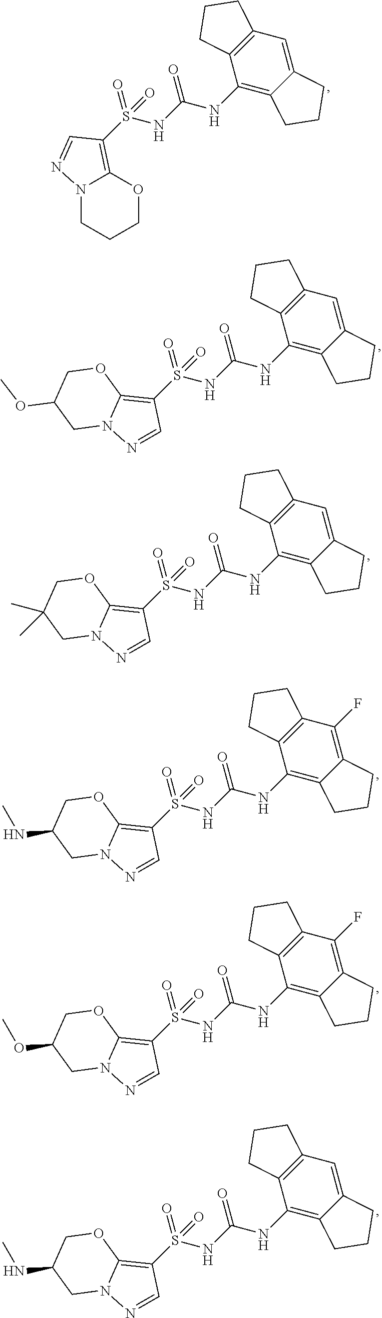 [SMILES: O=C(Nc1c2c(cc3c1CCC3)CCC2)NS(=O)(=O)c1cnn2c1OCCC2 ; COC1COc2c(S(=O)(=O)NC(=O)Nc3c4c(cc5c3CCC5)CCC4)cnn2C1 ; CC1(C)COc2c(S(=O)(=O)NC(=O)Nc3c4c(cc5c3CCC5)CCC4)cnn2C1 ; CN[C@H]1COc2c(S(=O)(=O)NC(=O)Nc3c4c(c(F)c5c3CCC5)CCC4)cnn2C1 ; CO[C@H]1COc2c(S(=O)(=O)NC(=O)Nc3c4c(c(F)c5c3CCC5)CCC4)cnn2C1 ; CN[C@H]1COc2c(S(=O)(=O)NC(=O)Nc3c4c(cc5c3CCC5)CCC4)cnn2C1] 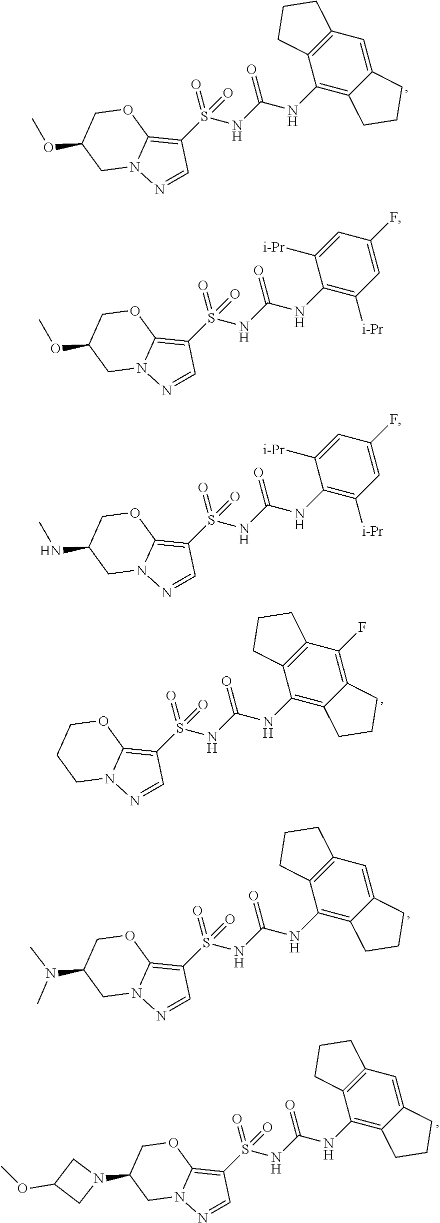 [SMILES: CO[C@H]1COc2c(S(=O)(=O)NC(=O)Nc3c4c(cc5c3CCC5)CCC4)cnn2C1 ; CO[C@H]1COc2c(S(=O)(=O)NC(=O)Nc3c(C(C)C)cc(F)cc3C(C)C)cnn2C1 ; CN[C@H]1COc2c(S(=O)(=O)NC(=O)Nc3c(C(C)C)cc(F)cc3C(C)C)cnn2C1 ; O=C(Nc1c2c(c(F)c3c1CCC3)CCC2)NS(=O)(=O)c1cnn2c1OCCC2 ; CN(C)[C@H]1COc2c(S(=O)(=O)NC(=O)Nc3c4c(cc5c3CCC5)CCC4)cnn2C1 ; COC1CN([C@H]2COc3c(S(=O)(=O)NC(=O)Nc4c5c(cc6c4CCC6)CCC5)cnn3C2)C1] 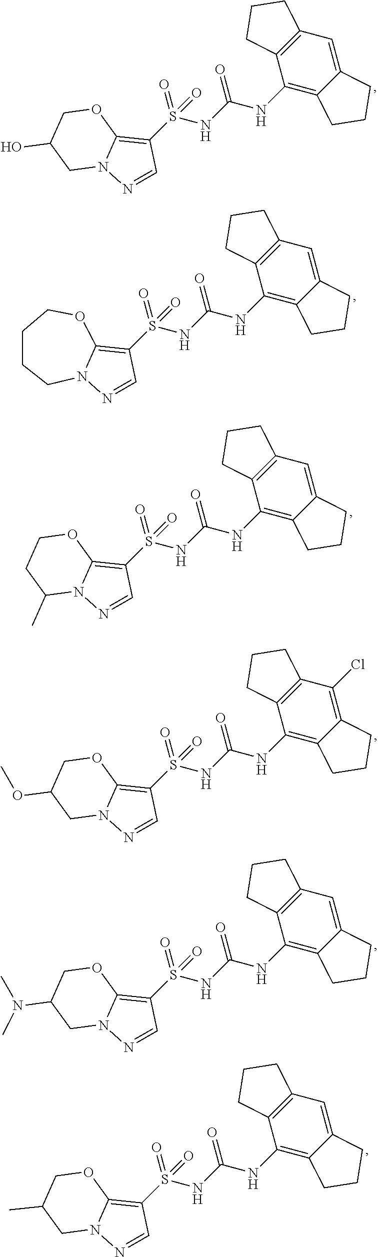 [SMILES: O=C(Nc1c2c(cc3c1CCC3)CCC2)NS(=O)(=O)c1cnn2c1OCC(O)C2 ; O=C(Nc1c2c(cc3c1CCC3)CCC2)NS(=O)(=O)c1cnn2c1OCCCC2 ; CC1CCOc2c(S(=O)(=O)NC(=O)Nc3c4c(cc5c3CCC5)CCC4)cnn21 ; COC1COc2c(S(=O)(=O)NC(=O)Nc3c4c(c(Cl)c5c3CCC5)CCC4)cnn2C1 ; CN(C)C1COc2c(S(=O)(=O)NC(=O)Nc3c4c(cc5c3CCC5)CCC4)cnn2C1 ; CC1COc2c(S(=O)(=O)NC(=O)Nc3c4c(cc5c3CCC5)CCC4)cnn2C1] 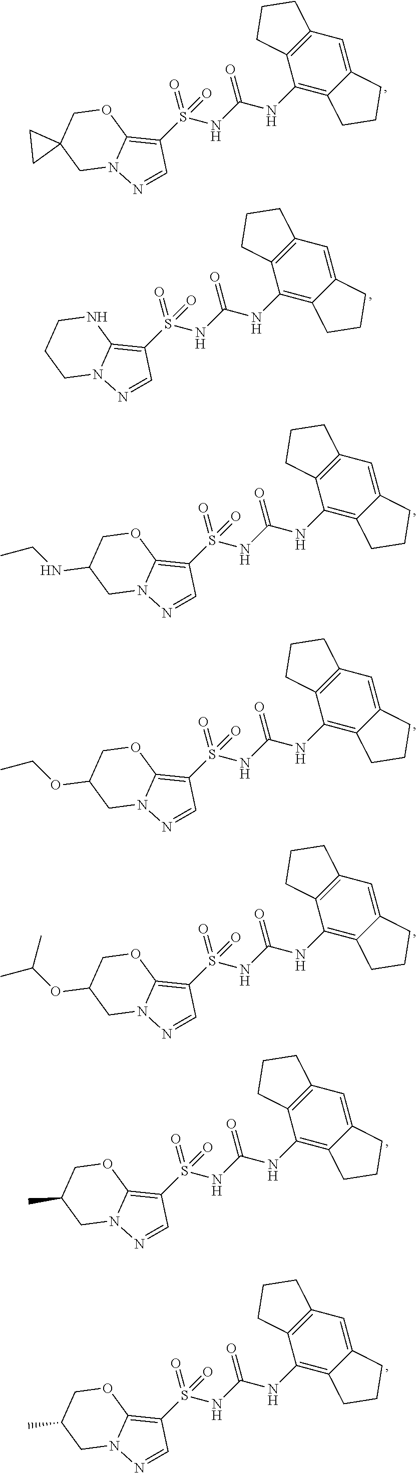 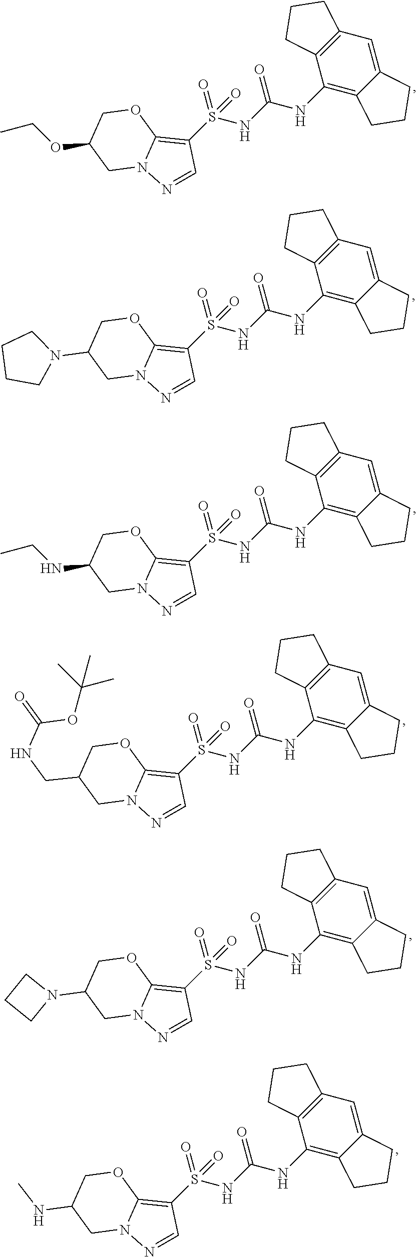 [SMILES: CCO[C@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCCC1)C2 ; CCN[C@H]1COc2c(S(=O)(=O)NC(=O)Nc3c4c(cc5c3CCC5)CCC4)cnn2C1 ; CC(C)(C)OC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1OCC(N1CCC1)C2 ; CNC1COc2c(S(=O)(=O)NC(=O)Nc3c4c(cc5c3CCC5)CCC4)cnn2C1] 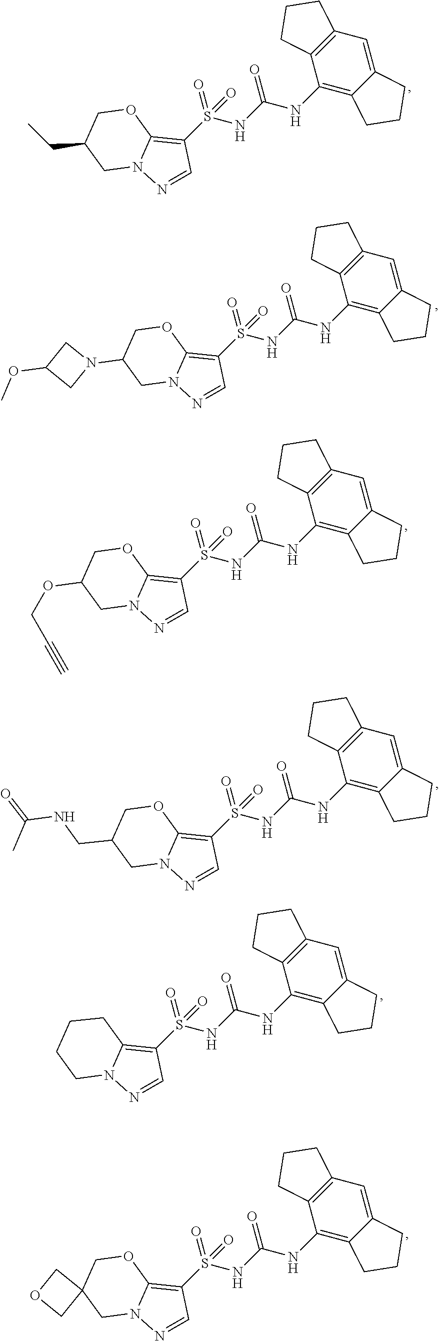 [SMILES: CC[C@H]1COc2c(S(=O)(=O)NC(=O)Nc3c4c(cc5c3CCC5)CCC4)cnn2C1 ; COC1CN(C2COc3c(S(=O)(=O)NC(=O)Nc4c5c(cc6c4CCC6)CCC5)cnn3C2)C1 ; C#CCOC1COc2c(S(=O)(=O)NC(=O)Nc3c4c(cc5c3CCC5)CCC4)cnn2C1 ; CC(=O)NCC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1CCCC2 ; O=C(Nc1c2c(cc3c1CCC3)CCC2)NS(=O)(=O)c1cnn2c1OCC1(COC1)C2] 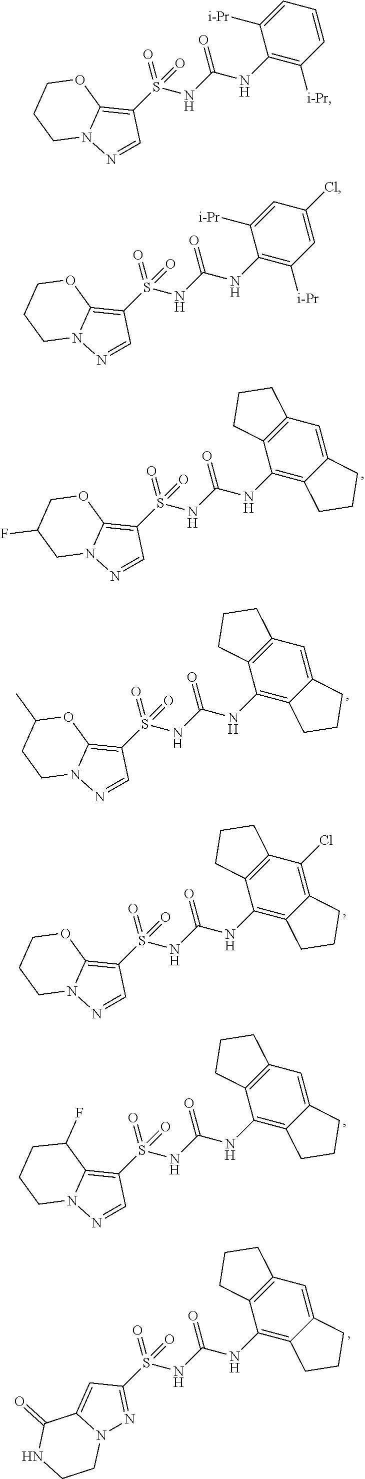 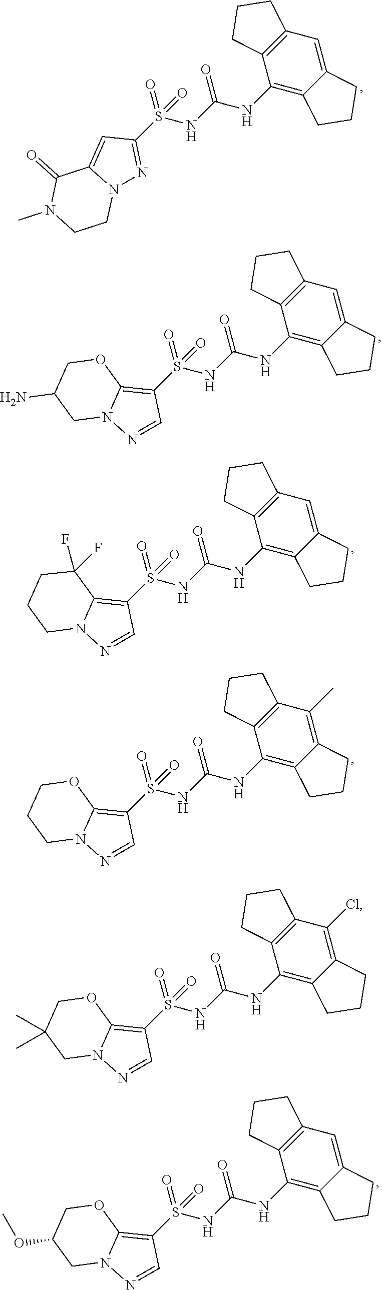 [SMILES: CN1CCn2nc(S(=O)(=O)NC(=O)Nc3c4c(cc5c3CCC5)CCC4)cc2C1=O ; NC1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1C(F)(F)CCC2 ; Cc1c2c(c(NC(=O)NS(=O)(=O)c3cnn4c3OCCC4)c3c1CCC3)CCC2 ; CC1(C)COc2c(S(=O)(=O)NC(=O)Nc3c4c(c(Cl)c5c3CCC5)CCC4)cnn2C1 ; CO[C@@H]1COc2c(S(=O)(=O)NC(=O)Nc3c4c(cc5c3CCC5)CCC4)cnn2C1] 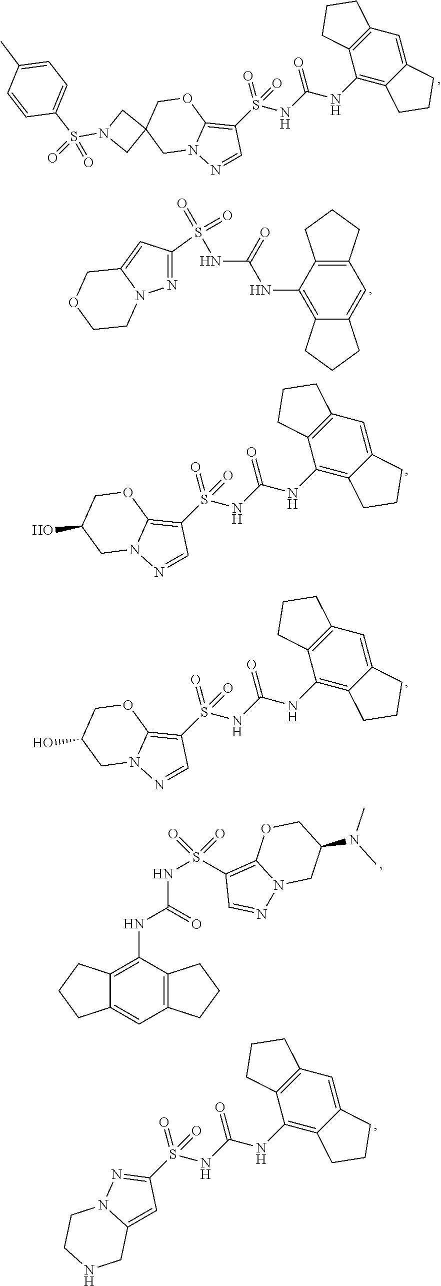 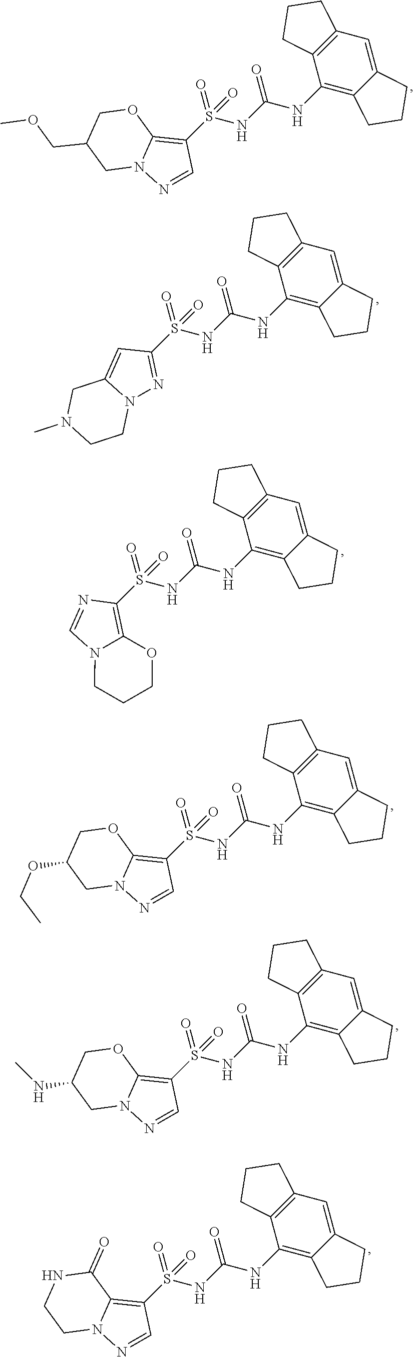 [SMILES: COCC1COc2c(S(=O)(=O)NC(=O)Nc3c4c(cc5c3CCC5)CCC4)cnn2C1 ; CN1CCn2nc(S(=O)(=O)NC(=O)Nc3c4c(cc5c3CCC5)CCC4)cc2C1 ; O=C(Nc1c2c(cc3c1CCC3)CCC2)NS(=O)(=O)c1ncn2c1OCCC2 ; CCO[C@@H]1COc2c(S(=O)(=O)NC(=O)Nc3c4c(cc5c3CCC5)CCC4)cnn2C1 ; CN[C@@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1cnn2c1C(=O)NCC2] 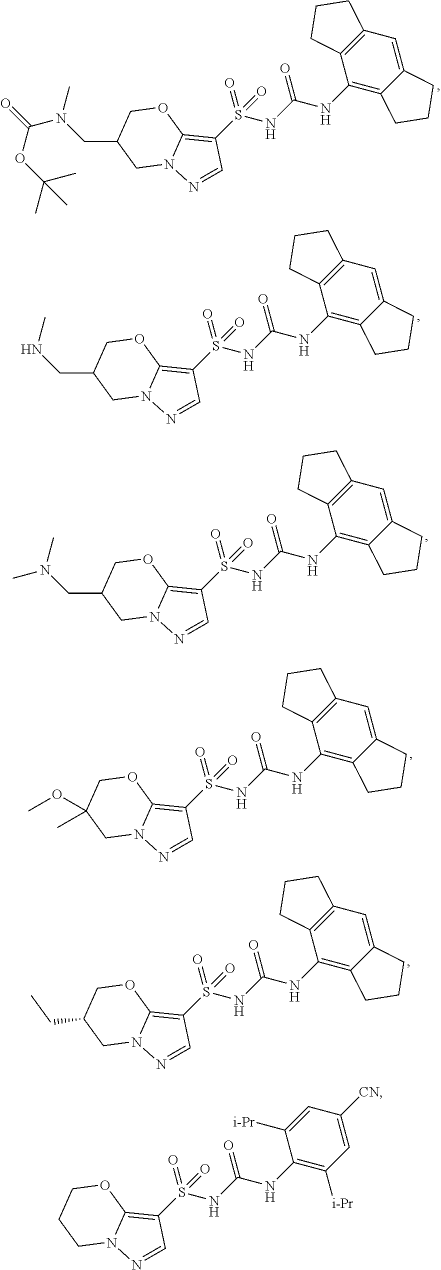 [SMILES: CN(CC1COc2c(S(=O)(=O)NC(=O)Nc3c4c(cc5c3CCC5)CCC4)cnn2C1)C(=O)OC(C)(C)C ; CNCC1COc2c(S(=O)(=O)NC(=O)Nc3c4c(cc5c3CCC5)CCC4)cnn2C1 ; CN(C)CC1COc2c(S(=O)(=O)NC(=O)Nc3c4c(cc5c3CCC5)CCC4)cnn2C1 ; COC1(C)COc2c(S(=O)(=O)NC(=O)Nc3c4c(cc5c3CCC5)CCC4)cnn2C1 ; CC[C@@H]1COc2c(S(=O)(=O)NC(=O)Nc3c4c(cc5c3CCC5)CCC4)cnn2C1 ; CC(C)c1cc(C#N)cc(C(C)C)c1NC(=O)NS(=O)(=O)c1cnn2c1OCCC2] 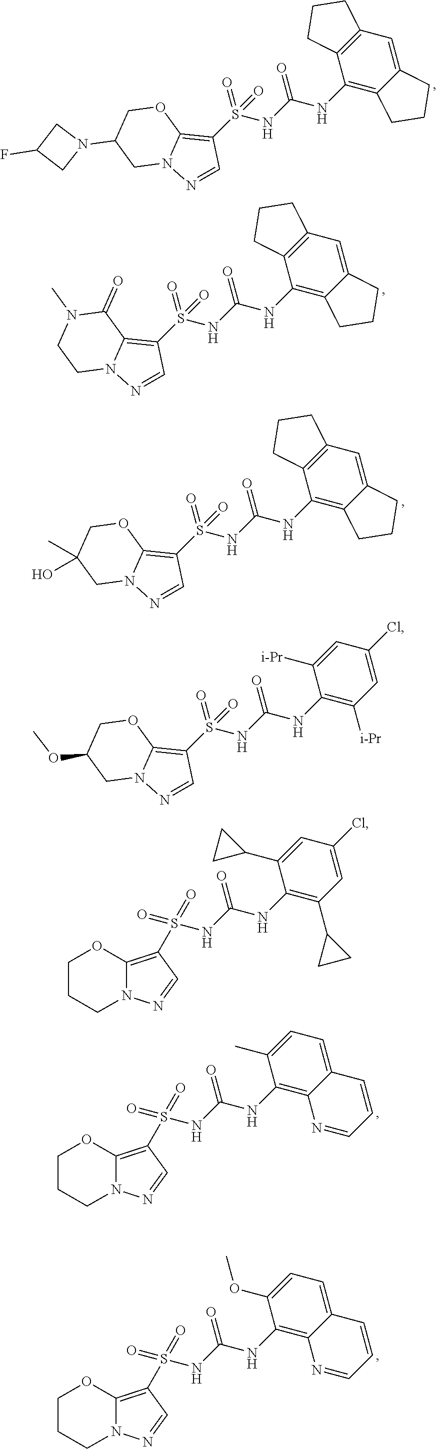 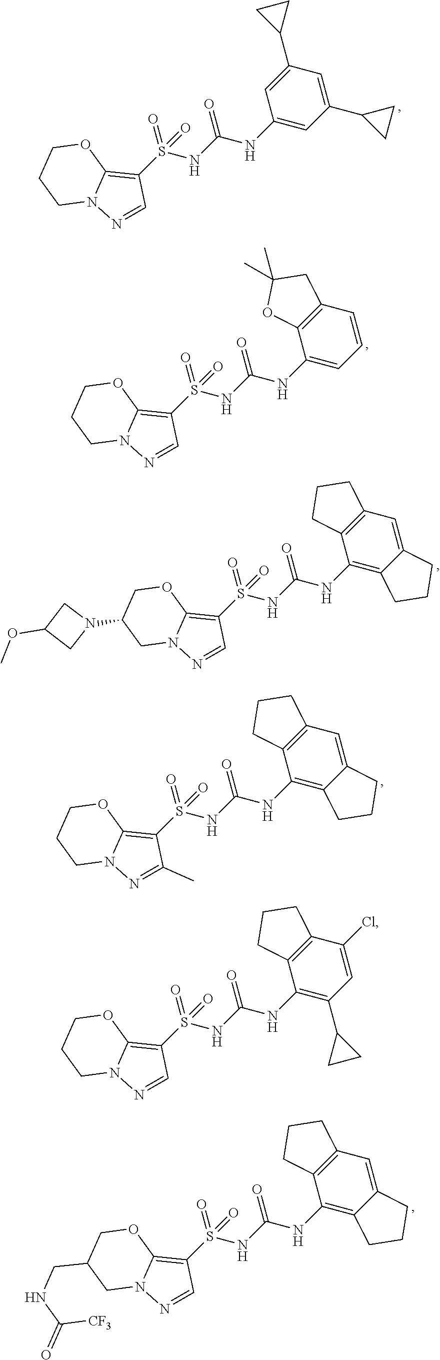 [SMILES: O=C(Nc1cc(C2CC2)cc(C2CC2)c1)NS(=O)(=O)c1cnn2c1OCCC2 ; CC1(C)Cc2cccc(NC(=O)NS(=O)(=O)c3cnn4c3OCCC4)c2O1 ; COC1CN([C@@H]2COc3c(S(=O)(=O)NC(=O)Nc4c5c(cc6c4CCC6)CCC5)cnn3C2)C1 ; Cc1nn2c(c1S(=O)(=O)NC(=O)Nc1c3c(cc4c1CCC4)CCC3)OCCC2 ; O=C(Nc1c(C2CC2)cc(Cl)c2c1CCC2)NS(=O)(=O)c1cnn2c1OCCC2 ; O=C(Nc1c2c(cc3c1CCC3)CCC2)NS(=O)(=O)c1cnn2c1OCC(CNC(=O)C(F)(F)F)C2] 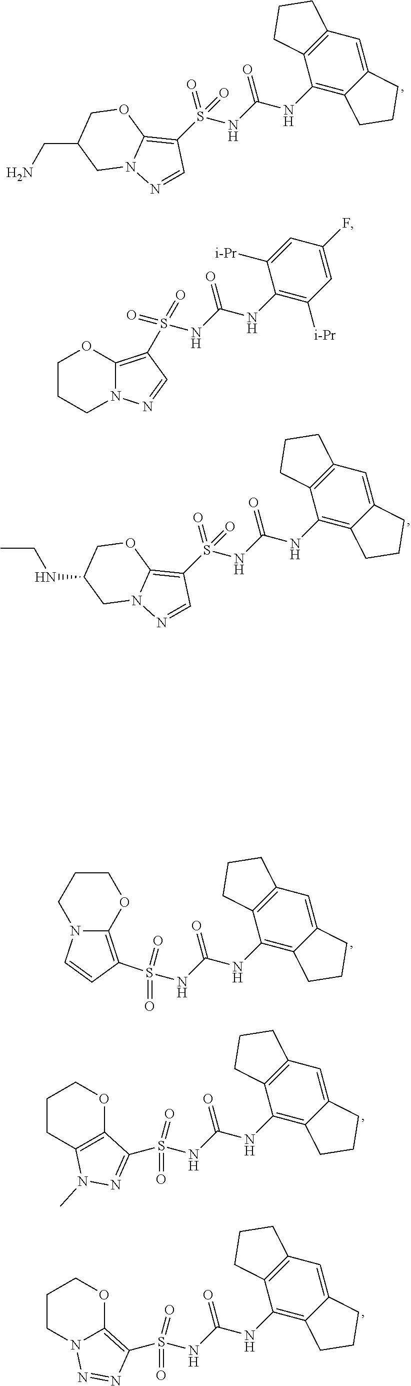 [SMILES: NCC1COc2c(S(=O)(=O)NC(=O)Nc3c4c(cc5c3CCC5)CCC4)cnn2C1 ; CC(C)c1cc(F)cc(C(C)C)c1NC(=O)NS(=O)(=O)c1cnn2c1OCCC2 ; CCN[C@@H]1COc2c(S(=O)(=O)NC(=O)Nc3c4c(cc5c3CCC5)CCC4)cnn2C1 ; O=C(Nc1c2c(cc3c1CCC3)CCC2)NS(=O)(=O)c1ccn2c1OCCC2 ; Cn1nc(S(=O)(=O)NC(=O)Nc2c3c(cc4c2CCC4)CCC3)c2c1CCCO2 ; O=C(Nc1c2c(cc3c1CCC3)CCC2)NS(=O)(=O)c1nnn2c1OCCC2] 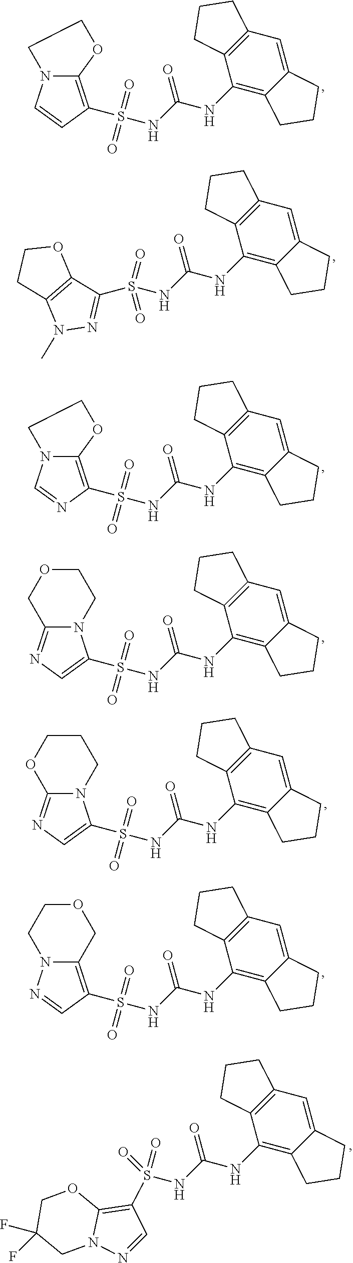 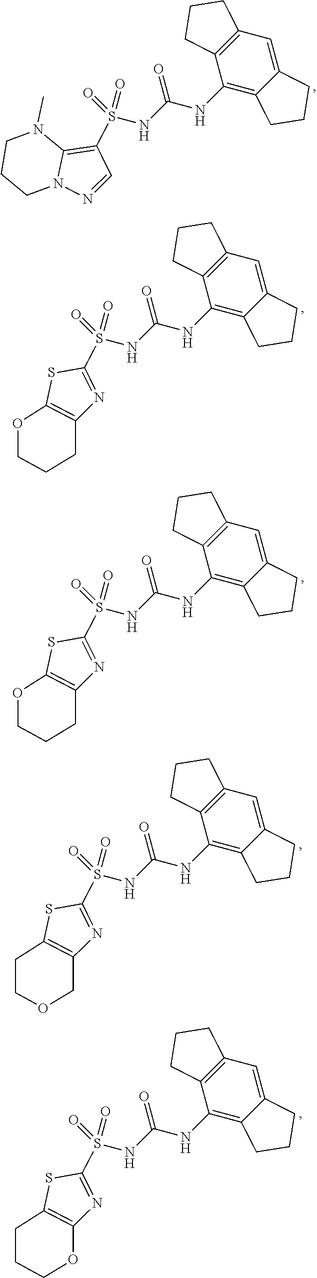 [SMILES: CN1CCCn2ncc(S(=O)(=O)NC(=O)Nc3c4c(cc5c3CCC5)CCC4)c21 ; O=C(Nc1c2c(cc3c1CCC3)CCC2)NS(=O)(=O)c1nc2c(s1)OCCC2 ; O=C(Nc1c2c(cc3c1CCC3)CCC2)NS(=O)(=O)c1nc2c(s1)OCCC2 ; O=C(Nc1c2c(cc3c1CCC3)CCC2)NS(=O)(=O)c1nc2c(s1)CCOC2 ; O=C(Nc1c2c(cc3c1CCC3)CCC2)NS(=O)(=O)c1nc2c(s1)CCCO2] 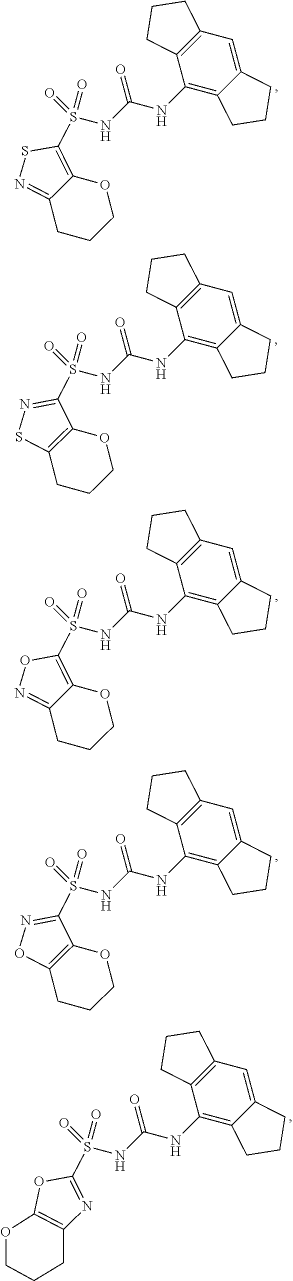 [SMILES: O=C(Nc1c2c(cc3c1CCC3)CCC2)NS(=O)(=O)c1snc2c1OCCC2 ; O=C(Nc1c2c(cc3c1CCC3)CCC2)NS(=O)(=O)c1nsc2c1OCCC2 ; O=C(Nc1c2c(cc3c1CCC3)CCC2)NS(=O)(=O)c1onc2c1OCCC2 ; O=C(Nc1c2c(cc3c1CCC3)CCC2)NS(=O)(=O)c1noc2c1OCCC2 ; O=C(Nc1c2c(cc3c1CCC3)CCC2)NS(=O)(=O)c1nc2c(o1)OCCC2] 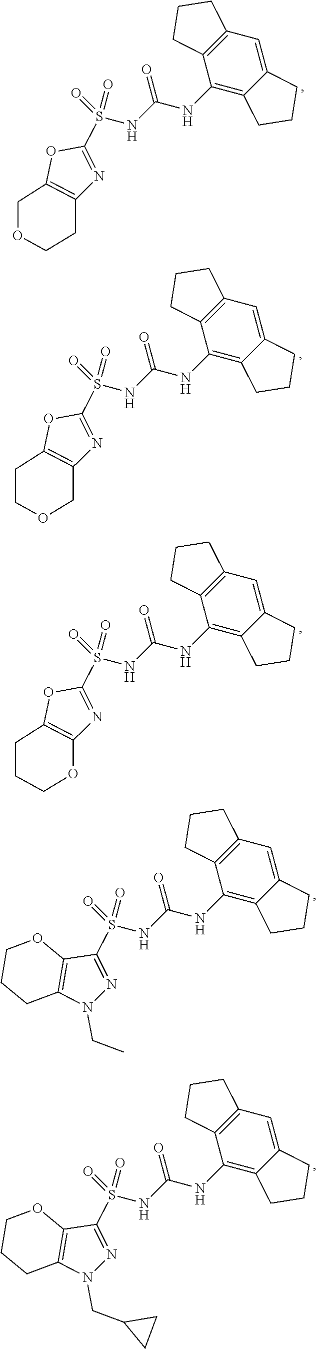 [SMILES: O=C(Nc1c2c(cc3c1CCC3)CCC2)NS(=O)(=O)c1nc2c(o1)COCC2 ; O=C(Nc1c2c(cc3c1CCC3)CCC2)NS(=O)(=O)c1nc2c(o1)CCOC2 ; O=C(Nc1c2c(cc3c1CCC3)CCC2)NS(=O)(=O)c1nc2c(o1)CCCO2 ; CCn1nc(S(=O)(=O)NC(=O)Nc2c3c(cc4c2CCC4)CCC3)c2c1CCCO2 ; O=C(Nc1c2c(cc3c1CCC3)CCC2)NS(=O)(=O)c1nn(CC2CC2)c2c1OCCC2] 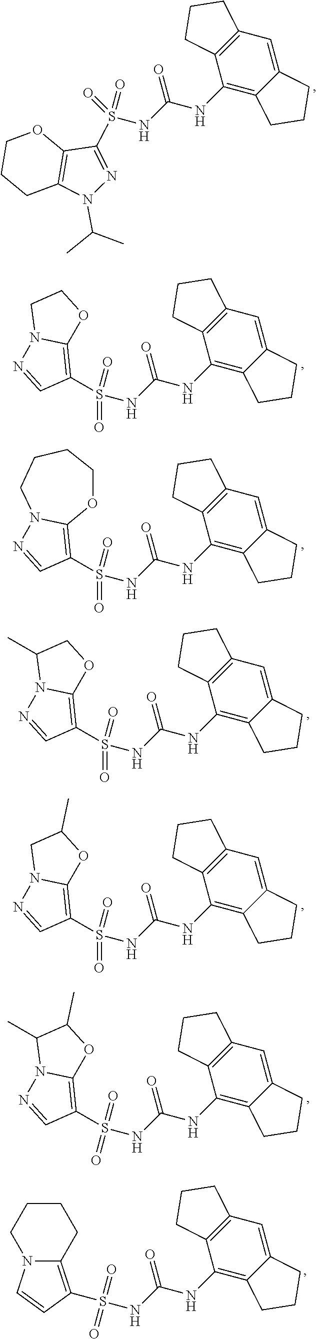 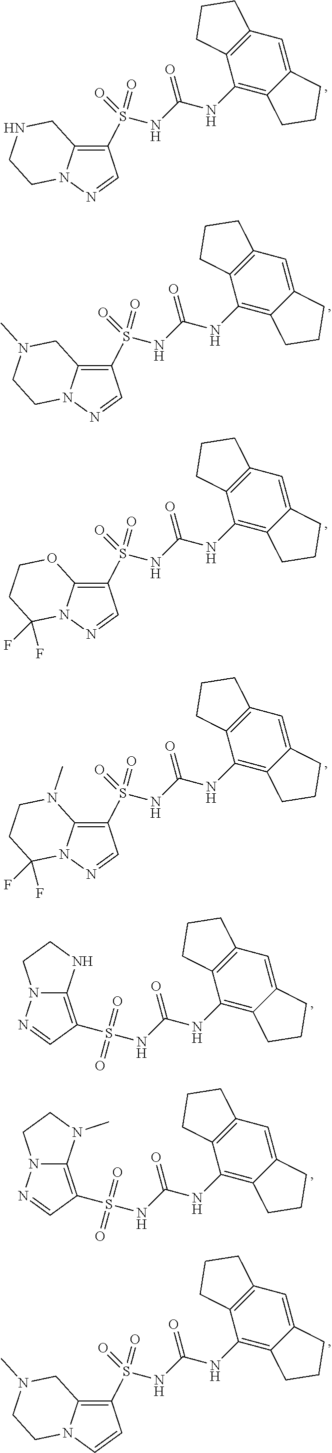 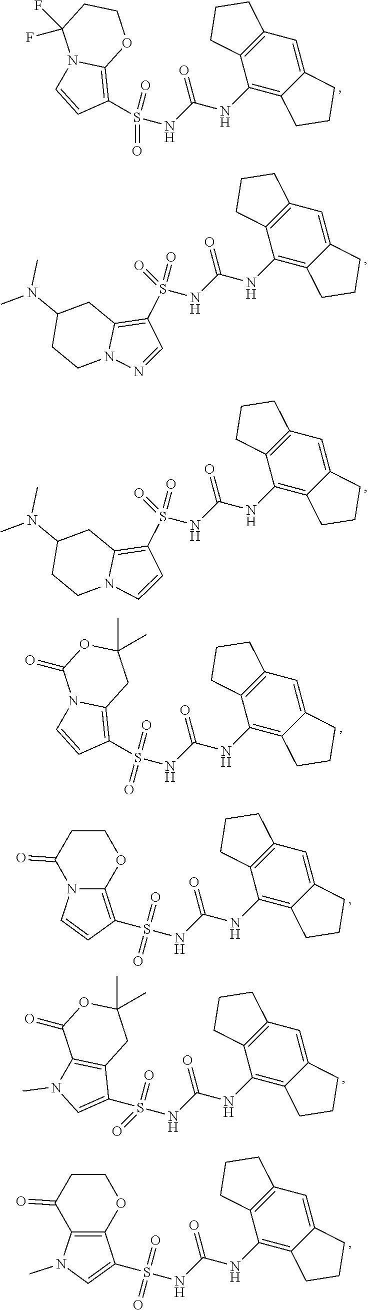 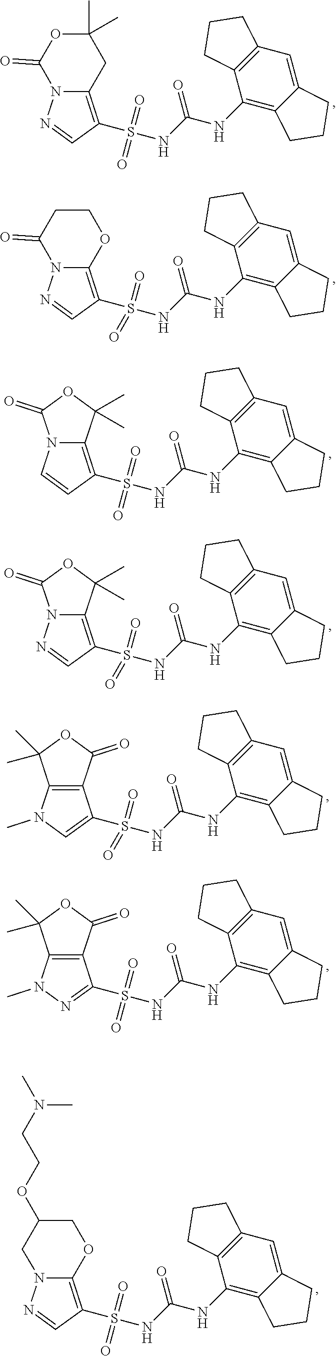 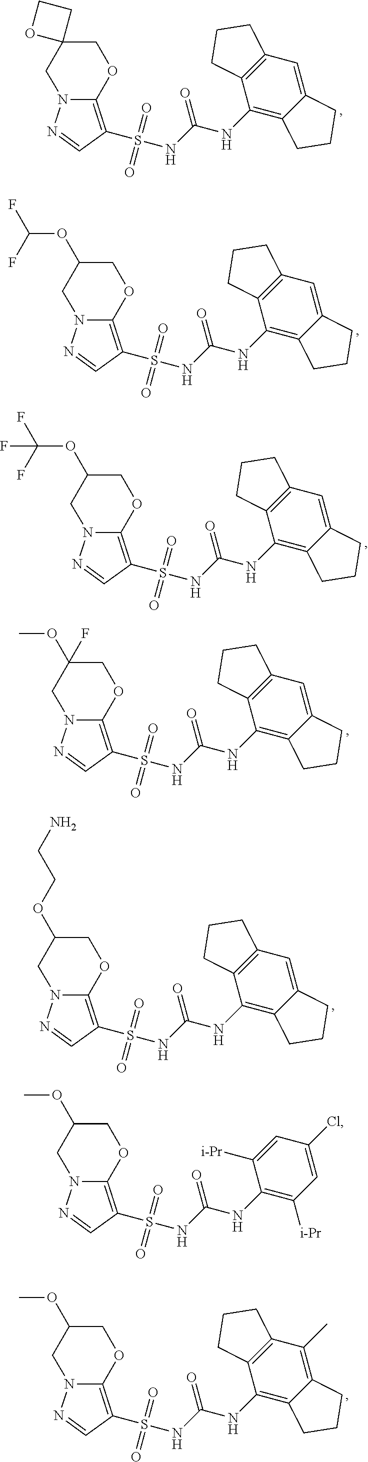 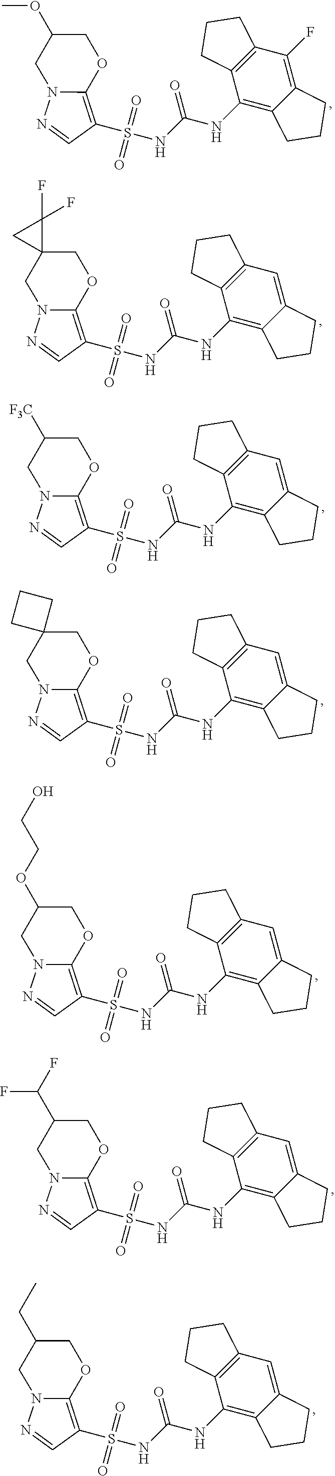 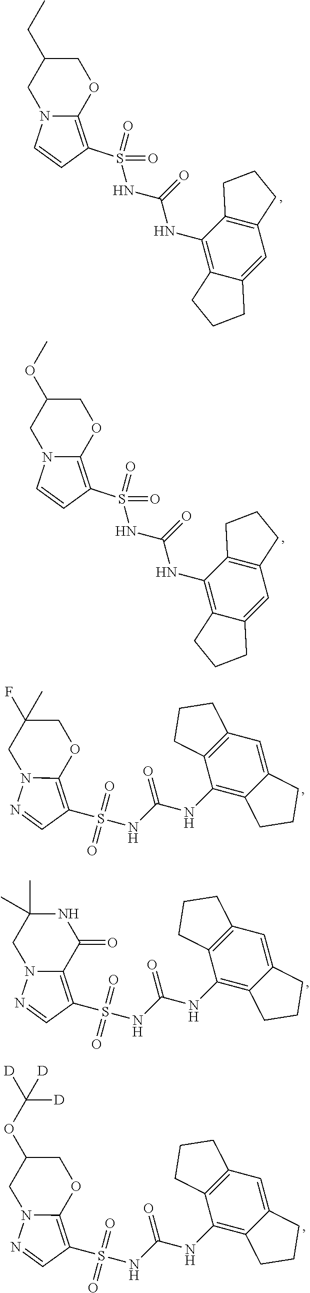 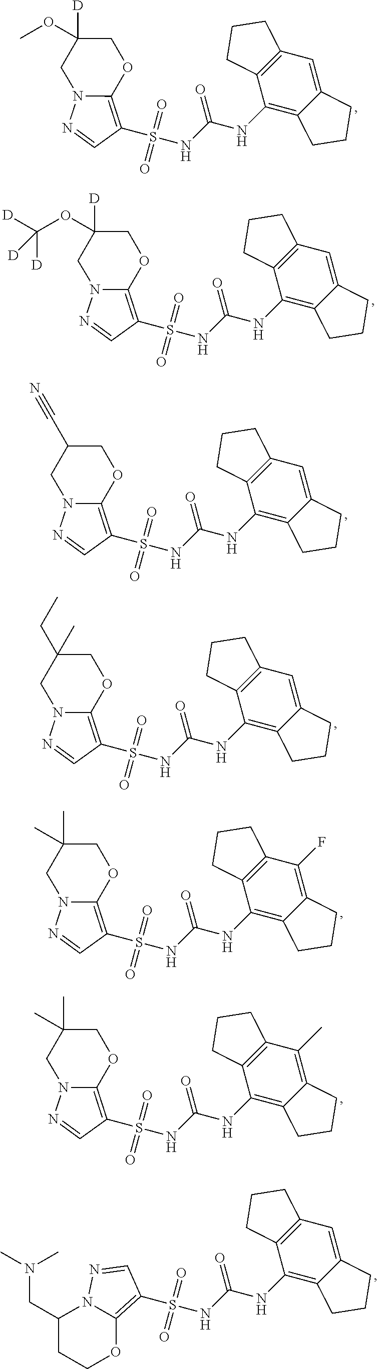 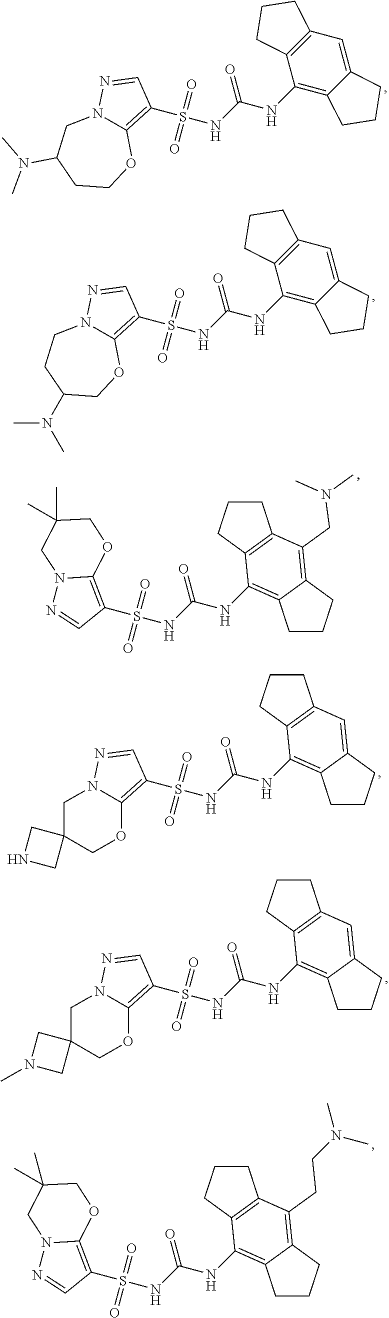 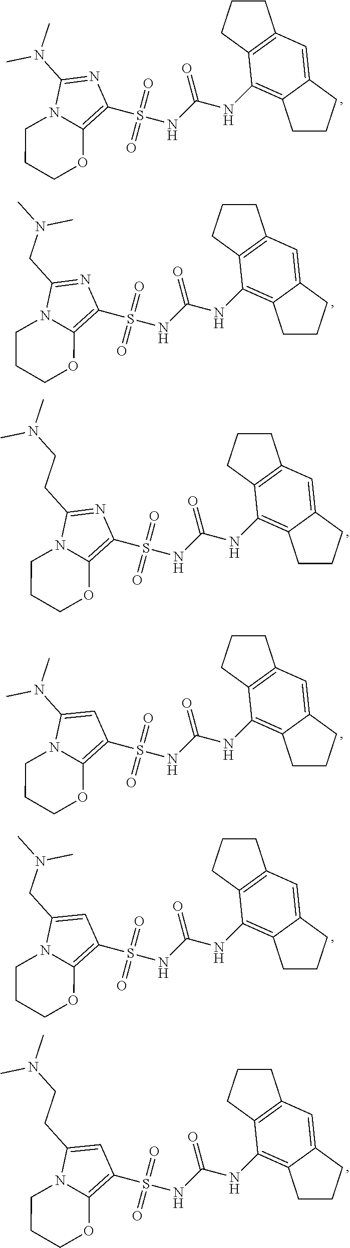 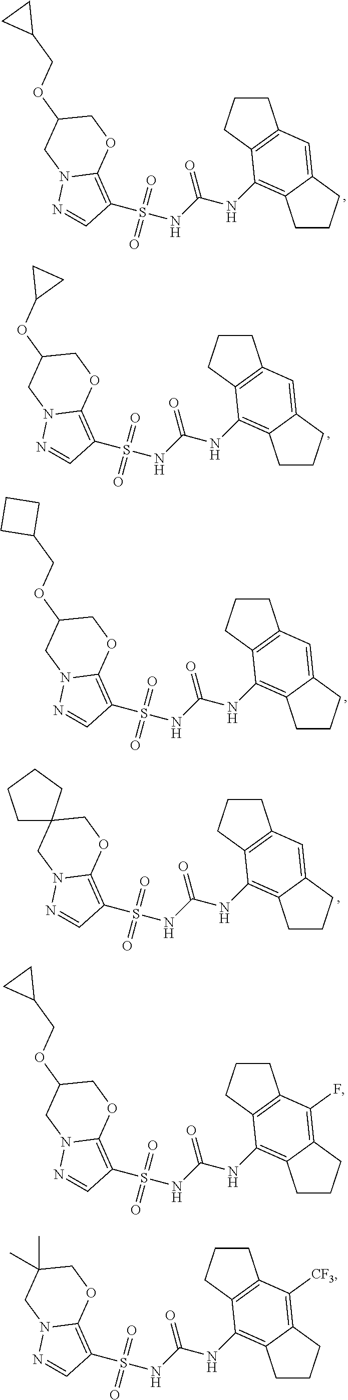 [SMILES: O=C(Nc1c2c(cc3c1CCC3)CCC2)NS(=O)(=O)c1cnn2c1OCC(OCC1CC1)C2 ; O=C(Nc1c2c(cc3c1CCC3)CCC2)NS(=O)(=O)c1cnn2c1OCC(OC1CC1)C2 ; O=C(Nc1c2c(cc3c1CCC3)CCC2)NS(=O)(=O)c1cnn2c1OCC(OCC1CCC1)C2 ; O=C(Nc1c2c(cc3c1CCC3)CCC2)NS(=O)(=O)c1cnn2c1OCC1(CCCC1)C2 ; O=C(Nc1c2c(c(F)c3c1CCC3)CCC2)NS(=O)(=O)c1cnn2c1OCC(OCC1CC1)C2 ; CC1(C)COc2c(S(=O)(=O)NC(=O)Nc3c4c(c(C(F)(F)F)c5c3CCC5)CCC4)cnn2C1] 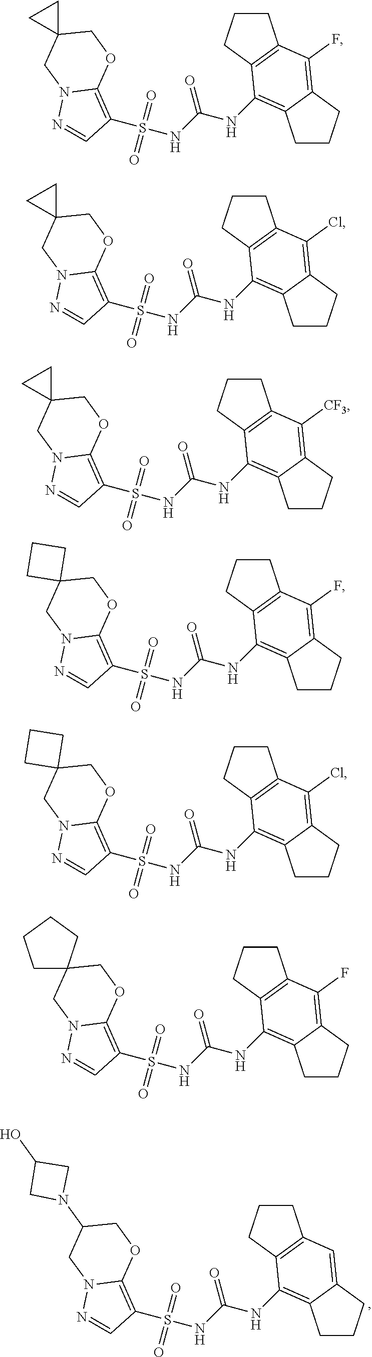 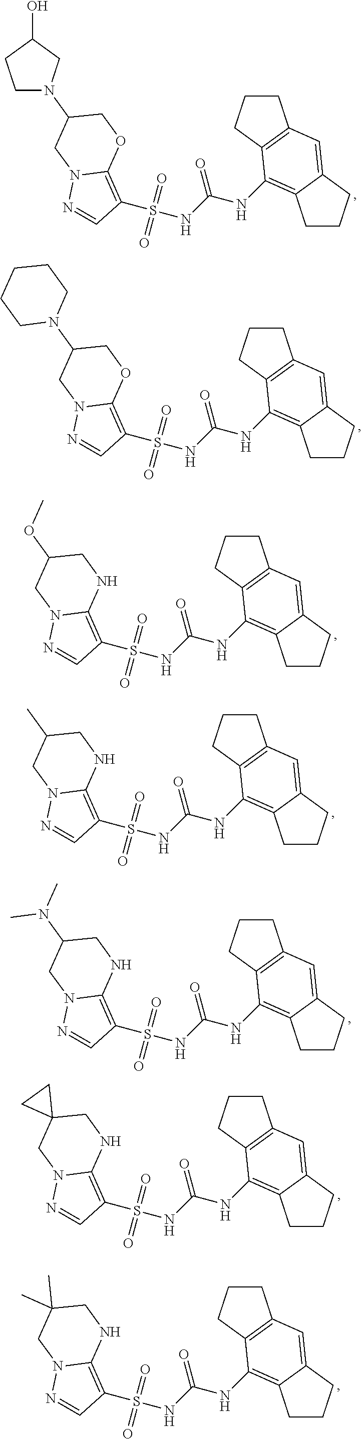 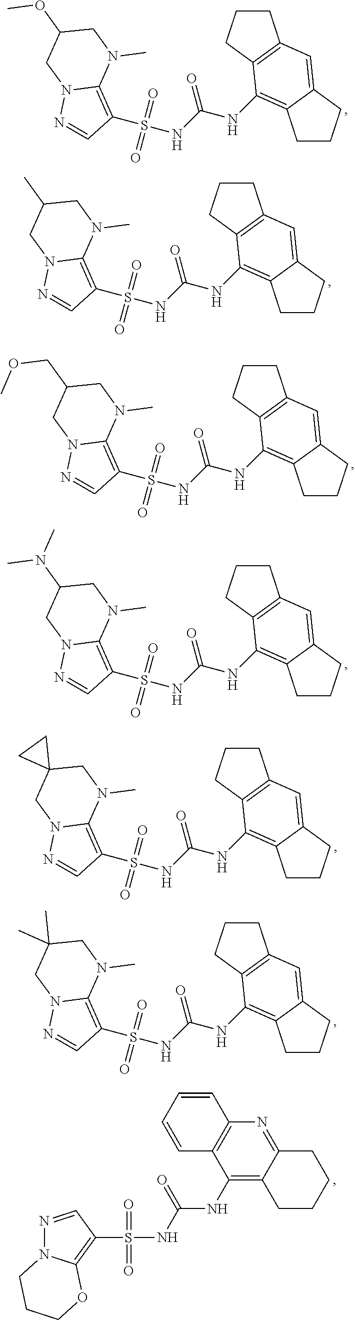 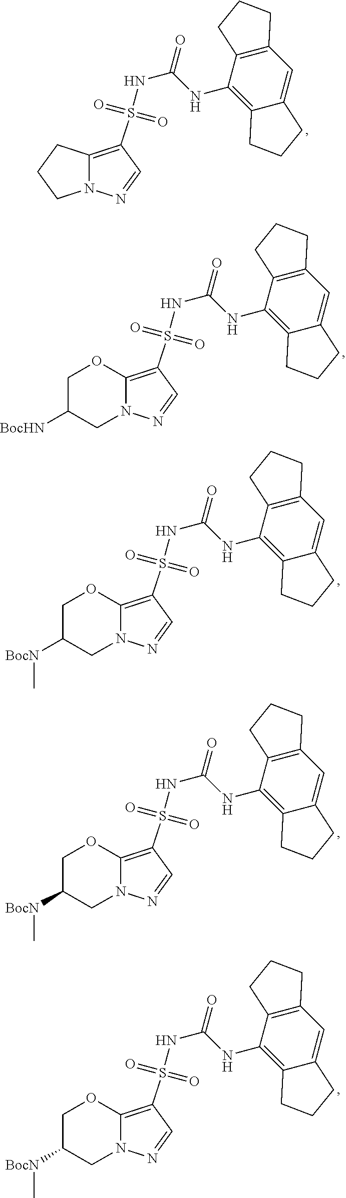 [SMILES: O=C(Nc1c2c(cc3c1CCC3)CCC2)NS(=O)(=O)c1cnn2c1CCC2 ; CC(C)(C)OC(=O)NC1COc2c(S(=O)(=O)NC(=O)Nc3c4c(cc5c3CCC5)CCC4)cnn2C1 ; CN(C(=O)OC(C)(C)C)C1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(cc5c3CCC5)CCC4)cnn2C1 ; CN(C(=O)OC(C)(C)C)[C@@H]1COc2c(S(=O)(=O)NC(=O)Nc3c4c(cc5c3CCC5)CCC4)cnn2C1] 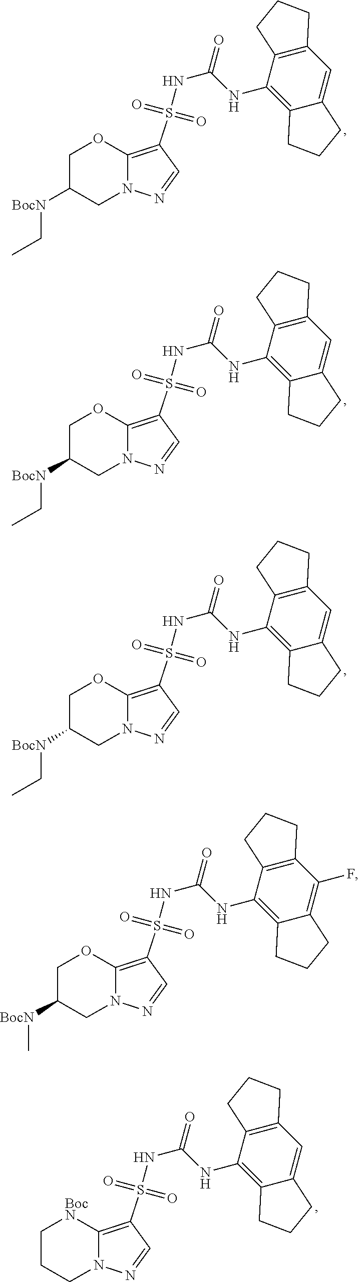 [SMILES: CCN(C(=O)OC(C)(C)C)C1COc2c(S(=O)(=O)NC(=O)Nc3c4c(cc5c3CCC5)CCC4)cnn2C1 ; CCN(C(=O)OC(C)(C)C)[C@H]1COc2c(S(=O)(=O)NC(=O)Nc3c4c(cc5c3CCC5)CCC4)cnn2C1 ; CCN(C(=O)OC(C)(C)C)[C@@H]1COc2c(S(=O)(=O)NC(=O)Nc3c4c(cc5c3CCC5)CCC4)cnn2C1 ; CN(C(=O)OC(C)(C)C)[C@H]1COc2c(S(=O)(=O)NC(=O)Nc3c4c(c(F)c5c3CCC5)CCC4)cnn2C1 ; CC(C)(C)OC(=O)N1CCCn2ncc(S(=O)(=O)NC(=O)Nc3c4c(cc5c3CCC5)CCC4)c21] 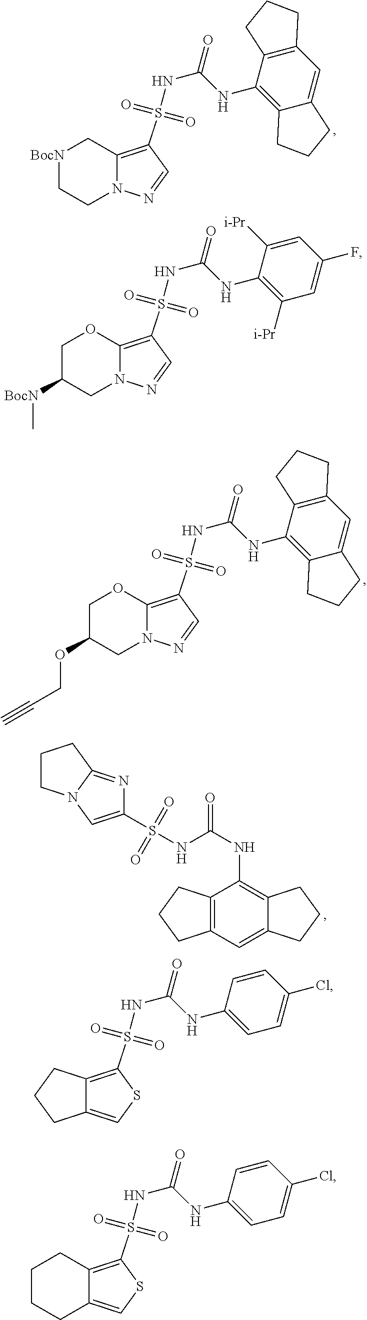 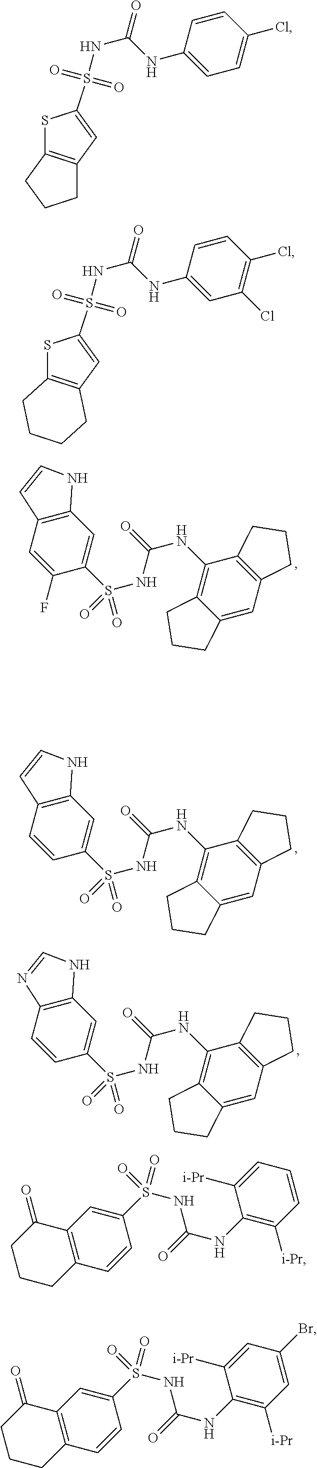 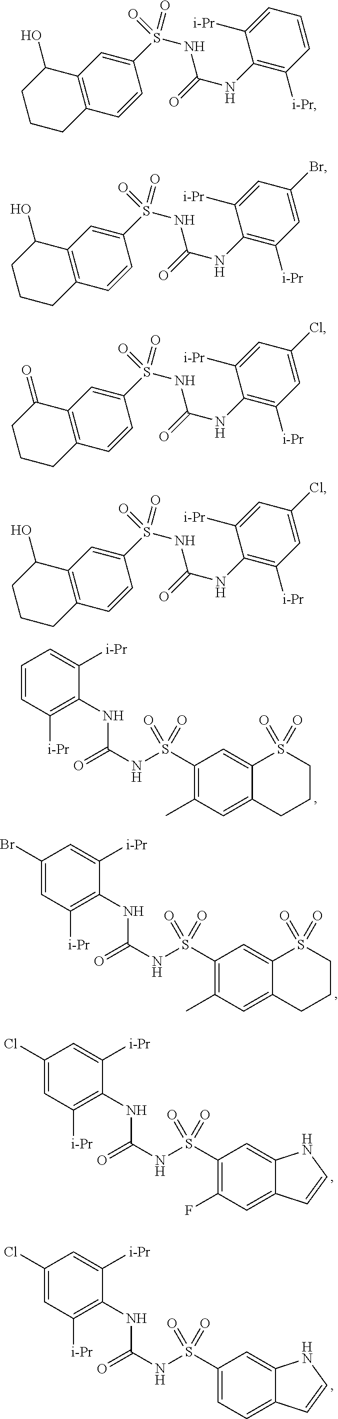 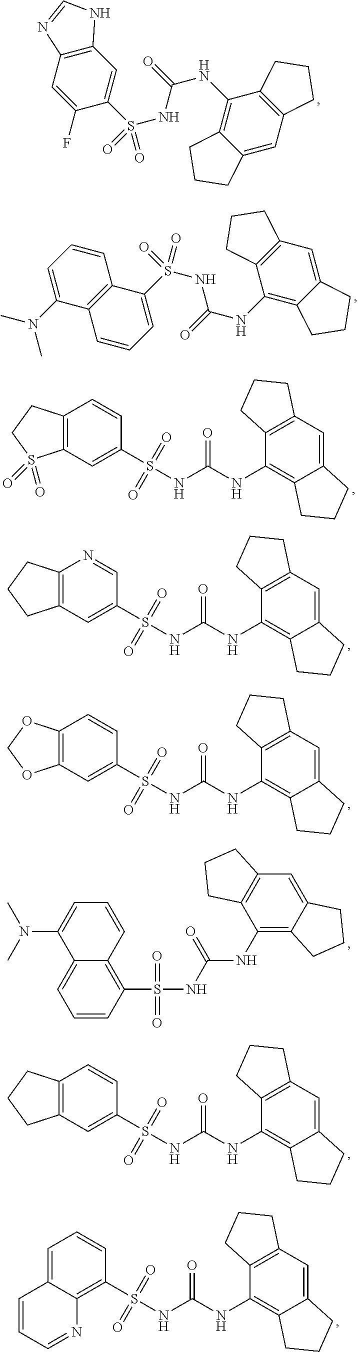 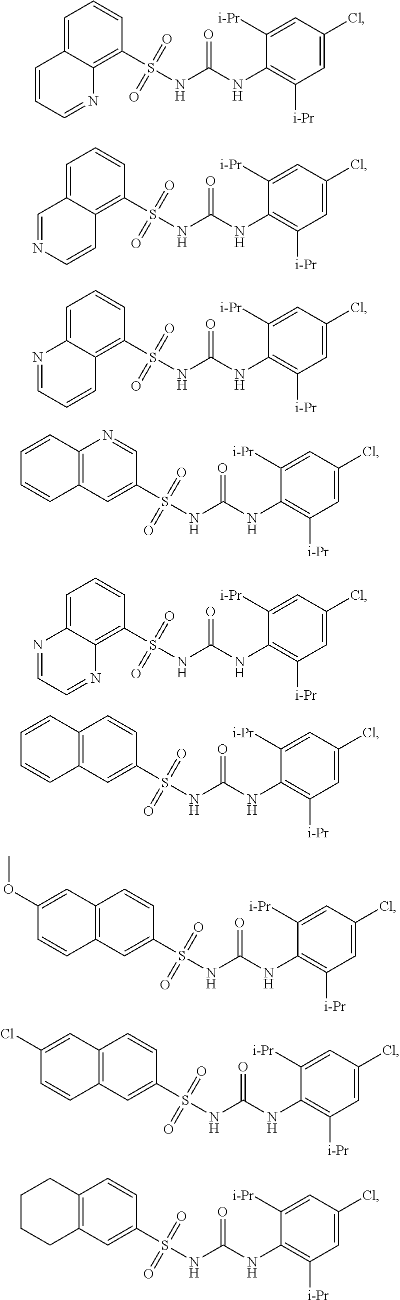 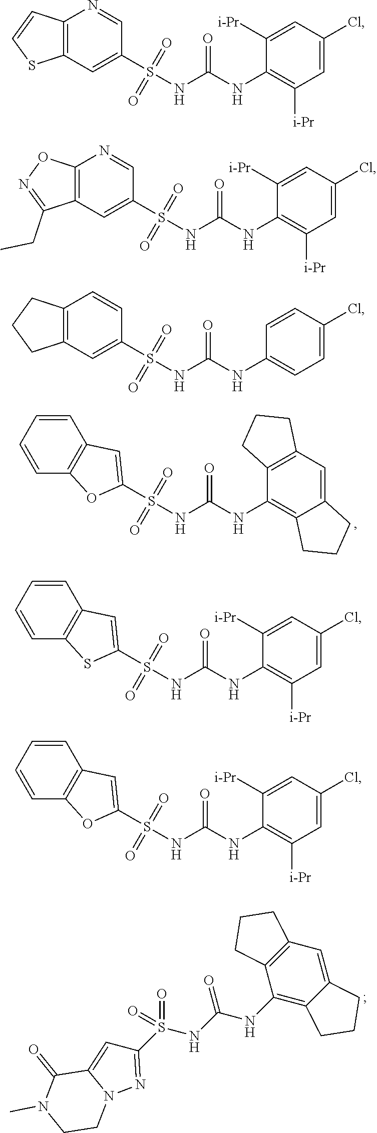
or a pharmaceutically acceptable salt thereof.

2. The compound of claim 1, wherein X⁴ is other than —X⁵—X⁶—; from two to four of R¹, R², R³, and R⁴ are present; and wherein at least two of the two to four R¹, R², R³, and R⁴ are on adjacent atoms, and taken together with the atoms connecting them, independently form a ring selected from the group consisting of:
(i) a monocyclic or bicyclic C₄-C₁₂ cycloalkyl ring optionally substituted with one or more R²⁰, and
(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰.

3. The compound of any one of claims 1-2, wherein when one of X¹ and X⁴ is N; the other of X¹ and X⁴ is other than O.

4. The compound of any one of claims 1-3, wherein X¹ is CR¹.

5. The compound of any one of claims 1-4, wherein X² is NR².

6. The compound of any one of claims 1-5, wherein X³ is N.

7. The compound of any one of claims 1-6, wherein X⁴ is CR⁴.

8. The compound of any one of claims 1-3, wherein X¹ is CR¹; X² is NR²; X³ is N; and X⁴ is CR⁴.

9. The compound of any one of claims 5-8, wherein R¹ and R², taken together with the atoms connecting them, form a monocyclic or bicyclic C₄-C₁₂ cycloalkyl ring optionally substituted with one or more R²⁰.

10. The compound of any one of claims 5-9, wherein R¹ and R², taken together with the atoms connecting them, form a monocyclic C₅-C₆ cycloalkyl ring optionally substituted with one or more R²⁰.

11. The compound of any one of claims 5-8, wherein R¹ and R², taken together with the atoms connecting them, form a monocyclic or bicyclic 5-to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰.

12. The compound of any one of claims 5-8 and 11, wherein R¹ and R², taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X¹ and X², and wherein the heterocycloalkyl ring is optionally substituted with one or more R²⁰.

13. The compound of claim 12, wherein the monocyclic 5- to-6-membered heterocycloalkyl ring contains 1 O atom.

14. The compound of any one of claims 7-13, wherein R⁴ is H or CH₃ (e.g., H).

15. The compound of any one of claims 1-3, wherein X¹ is N.

16. The compound of any one of claims 1-3 and 15, wherein X² is NR².

17. The compound of any one of claims 1-3 and 15-16, wherein X³ is CR³.

18. The compound of any one of claims 1-3 and 15-17, wherein X⁴ is CR⁴.

19. The compound of any one of claims 1-3, wherein X¹ is N; X² is NR²; X³ is CR³; and X⁴ is CR⁴.

20. The compound of any one of claims 17-19, wherein R² and R³, taken together with the atoms connecting them, form a monocyclic or bicyclic C₄-C₁₂ cycloalkyl ring optionally substituted with one or more R²⁰.

21. The compound of any one of claims 17-20, wherein R² and R³, taken together with the atoms connecting them, form a monocyclic C₅-C₆ cycloalkyl ring optionally substituted with one or more R²⁰.

22. The compound of any one of claims 17-19, wherein R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

23. The compound of any one of claims 17-19 and 22, wherein R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X² and X³, and wherein the heterocycloalkyl ring is optionally substituted with one or more R²⁰.

24. The compound of claim 23, wherein the monocyclic 5- to-6-membered heterocycloalkyl ring contains 1 O atom.

25. The compound of any one of claims 18-24, wherein R⁴ is H or CH₃.

26. The compound of any one of claims 1-3, wherein X¹ is S.

27. The compound of any one of claims 1-3 and 26, wherein X² is CR².

28. The compound of any one of claims 1-3 and 26-27, wherein X³ is CR³.

29. The compound of any one of claims 1-3 and 26-28, wherein X⁴ is CR⁴.

30. The compound of claim 29, wherein R³ and R⁴, taken together with the atoms connecting them, form a monocyclic or bicyclic C₄-C₁₂ cycloalkyl ring optionally substituted with one or more R²⁰.

31. The compound of any one of claims 29-30, wherein R³ and R⁴, taken together with the atoms connecting them, form a monocyclic C₅-C₆ cycloalkyl ring optionally substituted with one or more R²⁰.

32. The compound of claim 29, wherein R³ and R⁴, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

33. The compound of any one of claims 29 and 32, wherein R³ and R⁴, taken together with the atoms connecting them, form a monocyclic 5- to-6-membered heterocycloalkyl ring containing 1 heteroatom or heteroatomic group selected from O, N, NH, and NR¹³ (e.g., O, N, NH, or NCH₃), wherein the heteroatom or heteroatomic group is cumulative with the values selected for X³ and X⁴, and wherein the heterocycloalkyl ring is optionally substituted with one or more R²⁰.

34. The compound of claim 33, wherein the monocyclic 5- to-6-membered heterocycloalkyl ring contains 1 O atom.

35. The compound of any one of claims 27-34, wherein R² is H or CH₃.

36. The compound of any one of claims 1-3, wherein X¹ is S; and X⁴ is other than CR⁴.

37. The compound of any one of claims 1-3 and 36, wherein X² is CR².

38. The compound of any one of claims 1-3 and 36-37, wherein X³ is CR³.

39. The compound of any one of claims 1-3 and 36-38, wherein X⁴ is N.

40. The compound of any one of claims 1-3, wherein X¹ is S; X² is CR²; X³ is CR³; and X⁴ is N.

41. The compound of any one of claims 1-3 and 36-40, wherein R² and R³, taken together with the atoms connecting them, form a monocyclic or bicyclic C₄-C₁₂ cycloalkyl ring optionally substituted with one or more R²⁰.

42. The compound of any one of claims 1-3 and 36-41, wherein R² and R³, taken together with the atoms connecting them, form a monocyclic C₅-C₆ cycloalkyl ring optionally substituted with one or more R²⁰.

43. The compound of any one of claims 1-3 and 36-40, wherein R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

44. The compound of any one of claims 1-3, 36-40, and 43, wherein R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X² and X³, and wherein the heterocycloalkyl ring is optionally substituted with one or more R²⁰.

45. The compound of claim 44, wherein the monocyclic 5- to-6-membered heterocycloalkyl ring contains 1 O atom.

46. The compound of any one of claims 1-45, wherein each R²⁰ is independently selected from the group consisting of: hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom, wherein the ring is optionally substituted with OS(O)₂Ph.

47. The compound of any one of claims 1-46, wherein R²¹ is selected from the group consisting of halo (e.g., fluoro), NR⁸R⁹, C₂-C₆ alkynyl (e.g., ethynyl), and C₁-C₆ alkoxy (e.g., methoxy).

48. The compound of any one of claims 1-47, wherein o is 2 and p is 2.

49. The compound of any one of claims 1-48, wherein B is pyridyl.

50. The compound of claim 49, wherein B is 2-pyridyl.

51. The compound of claim 50, wherein the optionally substituted ring B is

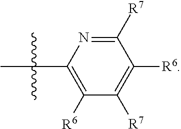

52. The compound of claim 51, wherein two pairs of R⁶ and R⁷ on adjacent atoms, taken together with the atoms connecting them, independently form a C₄-C₈ carbocyclic ring or a 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

53. The compound of claim 52, wherein the optionally substituted ring B is

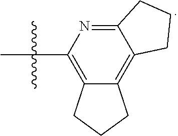 [SMILES: *c1nc2c(c3c1CCC3)CCC2]

54. The compound of claim 49, wherein B is 4-pyridyl.

55. The compound of claim 54, wherein the optionally substituted ring B is

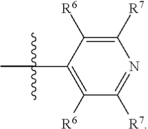

56. The compound of claim 55, wherein at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

57. The compound of claim 56, wherein the optionally substituted ring B is selected from the group consisting of:

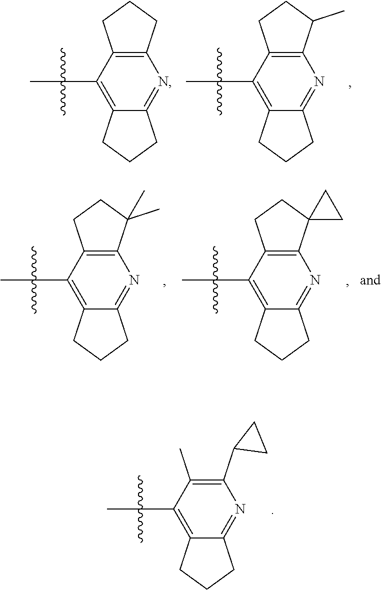

58. The compound of any one of claims 1-47, wherein, the optionally substituted ring B is

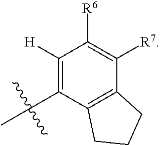

59. The compound of claim 58, wherein R⁶ and R⁷, taken together with the atoms connecting them, independently form one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

60. The compound of any one of claims 58-59, wherein

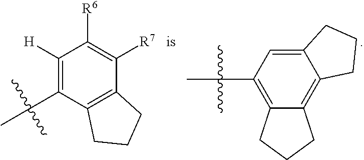

61. The compound of any one of claims 1-47, wherein the optionally substituted ring B is

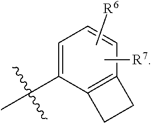

62. The compound of claim 61, wherein

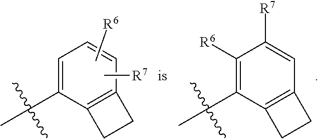

63. The compound of any one of claims 61-62, wherein R⁶ and R⁷, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

64. The compound of any one of claims 62-63, wherein 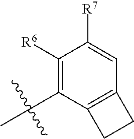
is selected from

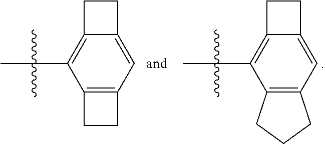 [SMILES: *c1c2c(cc3c1CC3)CC2 ; *c1c2c(cc3c1CC3)CCC2]

65. The compound of any one of claims 1-47, wherein the optionally substituted ring B is

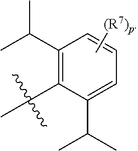

66. The compound of any one of claims 1 and 48-65, wherein 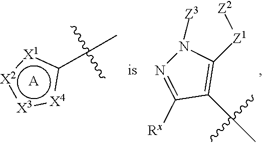
wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom wherein the ring is optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).

67. The compound of any one of claims 48-65, wherein 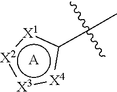
is selected from the group consisting of:

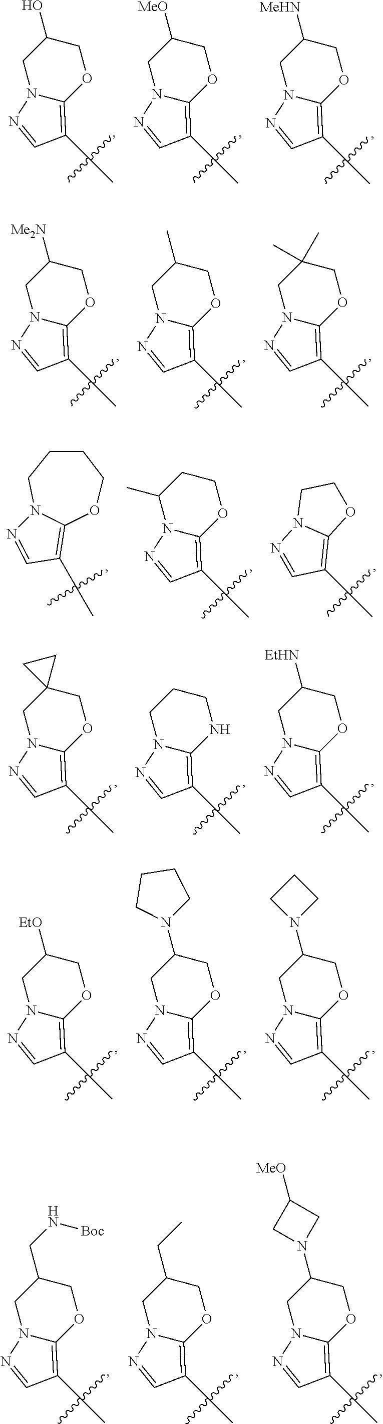

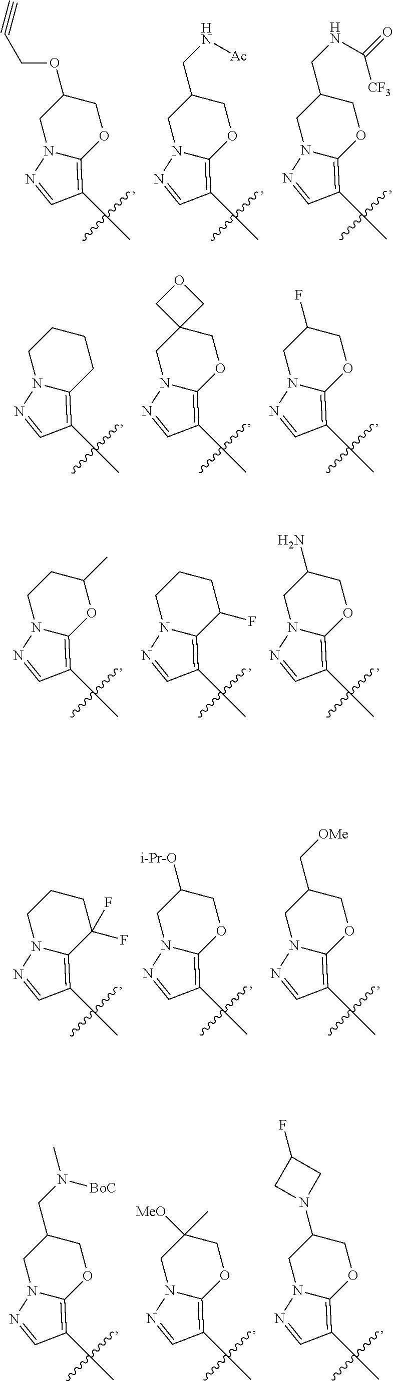

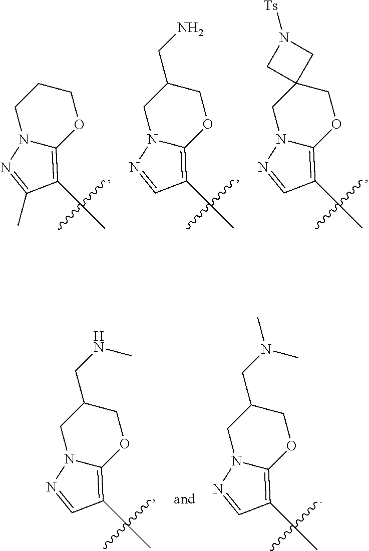 [SMILES: *c1c(C)nn2c1OCCC2 ; *c1cnn2c1OCC(CN)C2 ; *c1cnn2c1OCC1(CN(S(=O)(=O)c3ccc(C)cc3)C1)C2 ; *c1cnn2c1OCC(CNC)C2 ; *c1cnn2c1OCC(CN(C)C)C2]

68. The compound of any one of claims 48-65, wherein 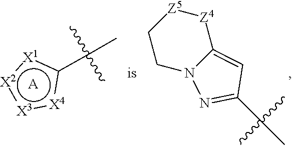
wherein Z⁴ is selected from the group consisting of —CH₂—, —C(O)—, and NH; Z⁵ is selected from the group consisting of O, NH, N—CH₃, and —CH₂—.

69. The compound of any one of claims 48-65, wherein 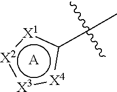
is selected from the group consisting of:

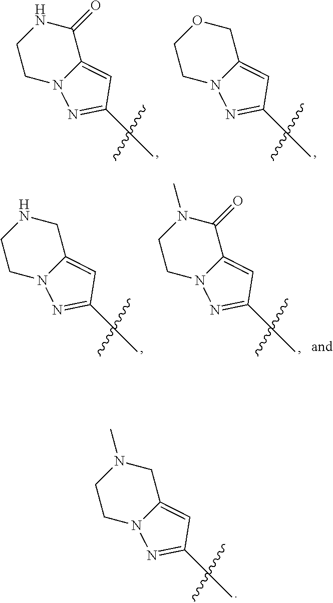 [SMILES: *c1cc2n(n1)CCNC2=O ; *c1cc2n(n1)CCOC2 ; *c1cc2n(n1)CCNC2 ; *c1cc2n(n1)CCN(C)C2=O ; *c1cc2n(n1)CCN(C)C2]

70. The compound of any one of claims 1-3 and 48-65, wherein X¹ is S.

71. The compound of any one of claims 1-3, 48-65, and 70, wherein X² is CR².

72. The compound of any one of claims 1-3, 48-65, and 70-71, wherein X³ is CR³.

73. The compound of any one of claims 1-3, 48-65, and 70-72, wherein X⁴ is CR⁴.

74. The compound of any one of claims 72-73, wherein R³ and R⁴ are taken together with the atoms connecting them to form a ring, wherein the ring is selected from the group consisting of:
(i) a monocyclic or bicyclic C₄-C₁₂ cycloalkyl ring optionally substituted with one or more R²⁰,
(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰,
(iii) a monocyclic or bicyclic C₆-C₁₀ aryl ring optionally substituted with one or more R²⁰, and
(iv) a monocyclic or bicyclic 5-12 membered heteroaryl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heteroaryl ring is optionally substituted with one or more R²⁰.

75. The compound of any one of claims 72-74, wherein R³ and R⁴ are taken together with the atoms connecting them to form a ring, wherein the ring is selected from the group consisting of:
(i) a monocyclic or bicyclic C₄-C₁₂ cycloalkyl ring optionally substituted with one or more R²⁰ and
(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

76. The compound of claim 75, wherein R³ and R⁴ are taken together with the atoms connecting them to form a ring, wherein the ring is selected from the group consisting of:
(i) a monocyclic C₅-C₆ cycloalkyl ring optionally substituted with one or more R²⁰ and
(ii) a monocyclic 5- to 6-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

77. The compound of any one of claims 72-75, wherein R³ and R⁴, taken together with the atoms connecting them, form a monocyclic or bicyclic C₄-C₁₂ cycloalkyl ring optionally substituted with one or more R²⁰.

78. The compound of any one of claims 72-75 and 77, wherein R³ and R⁴, taken together with the atoms connecting them, form a monocyclic C₅-C₆ cycloalkyl ring optionally substituted with one or more R²⁰.

79. The compound of any one of claims 72-75, wherein R³ and R⁴, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X³ and X⁴, and wherein the heterocycloalkyl ring is optionally substituted with one or more R²⁰.

80. The compound of any one of claims 72-75 and 79, wherein R³ and R⁴, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X³ and X⁴, and wherein the heterocycloalkyl ring is optionally substituted with one or more R²⁰.

81. The compound of any one of claims 72-75 and 79-81, wherein the ring is a monocyclic 5-6-membered heterocycloalkyl ring containing one heteroatom or heteroatomic group independently selected from O or NH, wherein the heteroatom or heteroatomic group is cumulative with the values selected for X³ and X⁴.

82. The compound of any one of claims 71-81, wherein R² is H or CH₃ (e.g., H).

83. The compound of any one of claims 80-81, wherein the ring is substituted with two independently selected R²⁰.

84. The compound of any one of claims 80-83, wherein R²⁰ is C₁-C₆ alkyl (e.g., methyl).

85. The compound of any one of claims 79-84, wherein 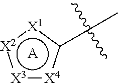
is selected from the group consisting of:

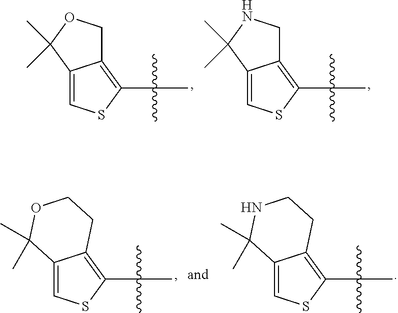 [SMILES: *c1scc2c1COC2(C)C ; *c1scc2c1CNC2(C)C ; *c1scc2c1CCOC2(C)C ; *c1scc2c1CCNC2(C)C]

86. The compound of any one of claims 70-73, wherein R² and R³ are taken together with the atoms connecting them to form a ring, wherein the ring is selected from the group consisting of:
(i) a monocyclic or bicyclic C₄-C₁₂ cycloalkyl ring optionally substituted with one or more R²⁰,
(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰,
(iii) a monocyclic or bicyclic C₆-C₁₀ aryl ring optionally substituted with one or more R²⁰, and
(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X² and X³, and wherein the heteroaryl ring is optionally substituted with one or more R²⁰.

87. The compound of claim 86, wherein the ring is a monocyclic 5-6 membered cycloalkyl ring optionally substituted with one or more R²⁰, or a monocyclic 5-6-membered heterocycloalkyl ring containing 1-3 heteroatoms and/or heteroatomic groups independently selected from O, N, NH, NR¹³, and S (e.g., O and NH), wherein the heteroatom or heteroatomic group is cumulative with the values selected for X² and X³, and wherein the heterocycloalkyl ring is optionally substituted with one or more R²⁰.

88. The compound of claim 86, wherein the ring is a monocyclic 5-6 membered cycloalkyl ring optionally substituted with one or more R²⁰.

89. The compound of any one of claims 86-88, wherein each R²⁰ is independently selected from hydroxy, C₁-C₆ alkyl (e.g., methyl), and NR⁸R⁹.

90. The compound of any one of claims 86-89, wherein 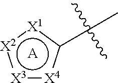
is selected from the group consisting of:

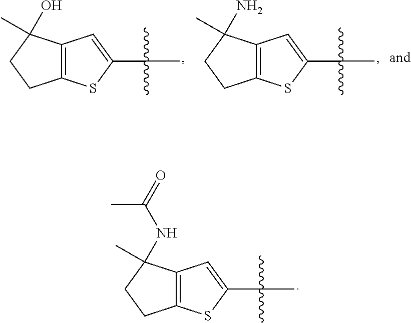 [SMILES: *c1cc2c(s1)CCC2(C)O ; *c1cc2c(s1)CCC2(C)N ; *c1cc2c(s1)CCC2(C)NC(C)=O]

91. The compound of any one of claims 70-90, where o is 2 and p is 2.

92. The compound of any one of claims 70-91, wherein the optionally substituted ring B is

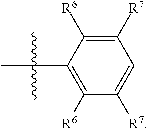

93. The compound of claim 92, wherein two pairs of R⁶ and R⁷ on adjacent atoms, taken together with the atoms connecting them, independently form two C₄-C₈ carbocyclic ring or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

94. The compound of any one of claims 91-93, wherein two pairs of R⁶ and R⁷ on adjacent atoms, taken together with the atoms connecting them, independently form two C₅ carbocyclic rings each optionally independently substituted with one or more R²⁷.

95. The compound of any one of claims 91-94, wherein the optionally substituted ring B is

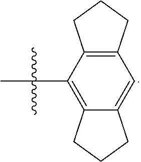 [SMILES: *c1c2c(cc3c1CCC3)CCC2]

96. The compound of any one of claims 70-90, wherein the optionally substituted ring B is

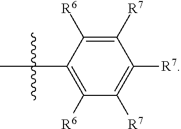

97. The compound of claim 96, wherein two pairs of R⁶ and R⁷ on adjacent atoms, taken together with the atoms connecting them, independently form two C₄-C₈ carbocyclic ring or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

98. The compound of any one of claims 96-97, wherein two pairs of R⁶ and R⁷ on adjacent atoms, taken together with the atoms connecting them, independently form two C₅ carbocyclic rings each optionally independently substituted with one or more R²⁷.

99. The compound of any one of claims 96-98, wherein the optionally substituted ring B is

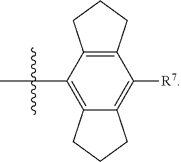

100. A compound of Formula AA 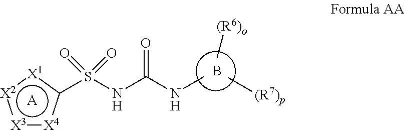
wherein the compound of Formula AA is selected from 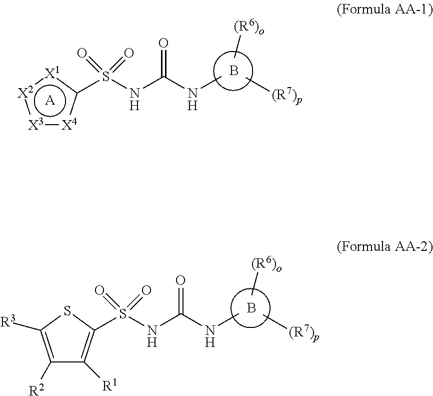
wherein
A is aromatic and charge neutral;
X¹ is O, S, N, CR¹, or NR¹;
X² is O, S, N, CR², or NR²;
X³ is O, S, N, CR³, or NR³;
X⁴ is O, S, N, CR⁴, NR⁴, or —X⁵—X⁶—;
X⁵ is N or CR⁵;
X⁶ is N or CR⁶;
wherein when X⁴ is —X⁵—X⁶, then:
X¹ is N or CR¹;
X² is N or CR²;
X³ is N or CR³;
when X⁴ is other than —X⁵—X⁶—, then 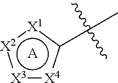
comprises at least one of CR¹, CR², CR³, and CR⁴;
when X⁴ is —X⁵—X⁶—, then 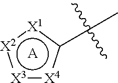
comprises at least two of CR¹, CR², CR³, CR⁵, and CR⁶;
wherein when X¹ is S, X⁴ is other than CR⁴;
wherein when X⁴ is S, X¹ is other than CR¹;
wherein when the compound of Formula AA is a compound of Formula AA-1, from two to four of R¹, R², R³, and R⁴ are present or from two to five of R¹, R², R³, R⁵, and R⁶ are present; and
wherein at least two of the two to four R¹, R², R³, and R⁴ or at least of two to five R¹, R², R³, R⁵, and R⁶ are on adjacent atoms;
wherein when the compound of Formula AA is a compound of Formula AA-1, any two adjacent R¹, R², R³, and R⁴ or any two adjacent R¹, R², R³, R⁵, and R⁶ are taken together with the atoms connecting them to form a ring; and wherein when the compound of Formula AA is a compound of Formula AA-2, any two adjacent R¹, R², and R³ are taken together with the atoms connecting them to form a ring; wherein the ring is selected from the group consisting of:
(i) a monocyclic or bicyclic C₄-C₁₂ cycloalkyl ring optionally substituted with one or more R²⁰,
(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heterocycloalkyl ring is optionally substituted with one or more R²⁰,
(iii) a monocyclic or bicyclic C₆-C₁₀ aryl ring optionally substituted with one or more R²⁰, and
(iv) a monocyclic or bicyclic 5-10 membered heteroaryl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴ when the compound of Formula AA is a compound of Formula AA-1, and wherein the heteroaryl ring is optionally substituted with one or more R²⁰;
R²⁰ is selected from the group consisting of: hydroxy, halo, oxo, C₁-C₆ alkyl optionally substituted with one or more R²¹, C₂-C₆ alkenyl optionally substituted with one or more R²¹, C₂-C₆ alkynyl optionally substituted with one or more R²¹, C₁-C₆ alkoxy optionally substituted with one or more R²¹, OC₃-C₁₀ cycloalkyl optionally substituted with one or more R²¹, NR⁸R⁹, ═NR¹⁰, CN, COOC₁-C₆ alkyl optionally substituted with one or more R²¹, S(O₂)C₆-C₁₀ aryl optionally substituted with one or more R²¹, OS(O₂)C₆-C₁₀ aryl optionally substituted with one or more R²¹, C₆-C₁₀ aryl optionally substituted with one or more R²¹, 5- to 10-membered heteroaryl optionally substituted with one or more R²¹, C₃-C₁₀ cycloalkyl optionally substituted with one or more R²¹, 3- to 10-membered heterocycloalkyl optionally substituted with one or more R²¹, and CONR⁸R⁹;
or at least one pair of R²⁰ on the same atom, taken together with the atom connecting them, independently forms a monocyclic or bicyclic C₄-C₁₂ cycloalkyl ring or at least one monocyclic or bicyclic 5- to-12-membered heterocycloalkyl ring containing 1-3 heteroatoms and/or heteroatomic groups independently selected from O, N, and S, wherein the cycloalkyl ring or heterocycloalkyl ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹;
R²¹ at each occurrence is independently selected from the group consisting of: hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
wherein the remaining R¹, R², R³, and R⁴, when present, are each independently selected from H, C₁-C₆ alkyl optionally substituted with one or more R²², C₁-C₆ haloalkyl optionally substituted with one or more R²², C₁-C₆ alkoxy optionally substituted with one or more R²², C₁-C₆ haloalkoxy optionally substituted with one or more R²², halo, CN, NO₂, COC₁-C₆ alkyl optionally substituted with one or more R²², CO—C₆-C₁₀ aryl optionally substituted with one or more R²², CO(5- to 10-membered heteroaryl) optionally substituted with one or more R²², CO₂C₁-C₆ alkyl optionally substituted with one or more R²², CO₂C₃-C₈ cycloalkyl optionally substituted with one or more R²², OCOC₁-C₆ alkyl optionally substituted with one or more R²², OCOC₆-C₁₀ aryl optionally substituted with one or more R²², OCO(5- to 10-membered heteroaryl) optionally substituted with one or more R²², OCO(3- to 7-membered heterocycloalkyl) optionally substituted with one or more R²², C₆-C₁₀ aryl optionally substituted with one or more R²², 5- to 10-membered heteroaryl optionally substituted with one or more R²², NH₂, NHC₁-C₆ alkyl optionally substituted with one or more R²², N(C₁-C₆ alkyl)₂ optionally substituted with one or more R²², NHCOC₁-C₆ alkyl optionally substituted with one or more R²², NHCOC₆-C₁₀ aryl optionally substituted with one or more R²², NHCO(5- to 10-membered heteroaryl) optionally substituted with one or more R²², NHCO(3- to 7-membered heterocycloalkyl) optionally substituted with one or more R²², NHCOC₂-C₆ alkynyl optionally substituted with one or more R²², NHCOOCC₁-C₆ alkyl optionally substituted with one or more R²², NH—(C═NR¹³)NR¹¹R¹², CONR⁸R⁹, SF₅, SC₁-C₆ alkyl optionally substituted with one or more R²², S(O₂)C₁-C₆ alkyl optionally substituted with one or more R²², S(O₂)NR¹¹R¹², S(O)C₁-C₆ alkyl optionally substituted with one or more R²², C₃-C₇ cycloalkyl optionally substituted with one or more R²², and 3- to 7-membered heterocycloalkyl optionally substituted with one or more R²²;
R²² at each occurrence is independently selected from the group consisting of: hydroxy, halo, CN, oxo, C₁-C₆ alkyl optionally substituted with one or more R²³, C₁-C₆ alkoxy optionally substituted with one or more R²³, NR⁸R⁹, ═NR¹⁰, COOC₁-C₆ alkyl optionally substituted with one or more R²³, CONR⁸R⁹, 3- to 7-membered heterocycloalkyl optionally substituted with one or more R²³, C₆-C₁₀ aryl optionally substituted with one or more R²⁴, 5- to 10-membered heteroaryl optionally substituted with one or more R²⁴, OCOC₁-C₆ alkyl optionally substituted with one or more R²³, OCOC₆-C₁₀ aryl optionally substituted with one or more R²⁴, OCO(5- to 10-membered heteroaryl) optionally substituted with one or more R²⁴, OCO(3- to 7-membered heterocycloalkyl) optionally substituted with one or more R²³, NHCOC₁-C₆ alkyl optionally substituted with one or more R²³, NHCOC₆-C₁₀ aryl optionally substituted with one or more R²⁴, NHCO(5- to 10-membered heteroaryl) optionally substituted with one or more R²⁴, NHCO(3- to 7-membered heterocycloalkyl) optionally substituted with one or more R²³, and NHCOC₂-C₆ alkynyl optionally substituted with one or more R²³;
R²³ at each occurrence is independently selected from the group consisting of: hydroxy, halo, NR⁸R⁹, C₁-C₆ alkyl, OC₁-C₆ alkyl, and oxo;
R²⁴ at each occurrence is independently selected from the group consisting of: hydroxy, halo, NR⁸R⁹, C₁-C₆ alkyl, and OC₁-C₆ alkyl;
wherein when the compound is a compound of Formula AA-1, 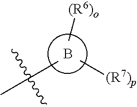
is selected from the group consisting of: 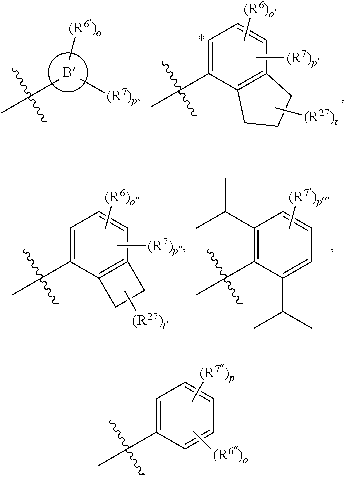
wherein * denotes that the ring position the * is closest to is unsubstituted;
B is a 5-10-membered heteroaryl or C₆-C₁₀ aryl;
B′ is a 5-6-membered heteroaryl, wherein when the 5-6 membered heteroaryl contains two or three nitrogen ring members, the 5-6-membered heteroaryl additionally contains one or more non-nitrogen heteroatom or heteroatomic group ring members; 5-pyrimidinyl; 6-pyrimidinyl;
pyridazinyl; pyrazinyl; 1,2,3-triazinyl; 1,2,4-triazinyl; tetrazinyl; imidazolyl; pyrazolyl; 1,2,3-triazolyl; tetrazolyl; or C₇-C₁₀ aryl;
o=1 or 2;
p=0, 1, 2, or 3;
o′=0 or 1;
p′=0 or 1;
o″=0 or 1;
p″=0, 1, or 2;
p′″=1, 2, or 3;
t is 0, 1, 2, 3, 4, 5, or 6;
t′ is 0, 1, 2, 3, or 4;
R⁶ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{6′} at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R⁷ at each occurrence is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
R^{7′}, at each occurrence, is independently selected from C₁-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, I, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₃-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵,
each occurrence of R^{6″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, F, Br, I, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
wherein when 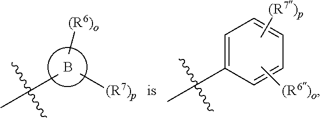
at least one R is ortho to the bond connecting the B ring to the NH(CO) group of Formula AA;
each occurrence of R^{7″} is independently selected from C₁-C₂ alkyl, C₄-C₆ alkyl optionally substituted with one or more R²⁵, C₁-C₆ haloalkyl optionally substituted with one or more R²⁵, C₁-C₆ alkoxy optionally substituted with one or more R²⁵, C₁-C₆ haloalkoxy optionally substituted with one or more R²⁵, halo, CN, NO₂, COC₁-C₆ alkyl optionally substituted with one or more R²⁵, CO₂C₁-C₆ alkyl optionally substituted with one or more R²⁵, CO₂C₃-C₈ cycloalkyl optionally substituted with one or more R²⁵, OCOC₁-C₆ alkyl optionally substituted with one or more R²⁵, OCOC₆-C₁₀ aryl optionally substituted with one or more R²⁵, OCO(5- to 10-membered heteroaryl) optionally substituted with one or more R²⁵, OCO(3- to 7-membered heterocycloalkyl) optionally substituted with one or more R²⁵, C₆-C₁₀ aryl optionally substituted with one or more R²⁵, 5- to 10-membered heteroaryl optionally substituted with one or more R²⁵, NH₂, NHC₁-C₆ alkyl optionally substituted with one or more R²⁵, N(C₁-C₆ alkyl)₂ optionally substituted with one or more R²⁵, CONR⁸R⁹, SF₅, SC₁-C₆ alkyl optionally substituted with one or more R²⁵, S(O₂)C₁-C₆ alkyl optionally substituted with one or more R²⁵, C₄-C₁₀ cycloalkyl optionally substituted with one or more R²⁵, 3- to 10-membered heterocycloalkyl optionally substituted with one or more R²⁵, and C₂-C₆ alkenyl optionally substituted with one or more R²⁵;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷;
or at least one pair of R^{6′} and R⁷ on adjacent atoms, taken together with the atoms connecting them, independently form at least one C₄-C₈ cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷;
or at least one pair of R^{6″} and R^{7″} on adjacent atoms, taken together with the atoms connecting them, independently form at least one C₄ or C₆-C₈ carbocyclic ring, wherein the carbocyclic ring is optionally independently substituted with one or more R²⁷;
R²⁵ at each occurrence is independently selected from the group consisting of: hydroxy, halo, CN, oxo, C₁-C₆ alkyl optionally substituted with one or more R²⁶, C₁-C₆ alkoxy optionally substituted with one or more R²⁶, NR⁸R⁹, ═NR¹⁰, COOC₁-C₆ alkyl optionally substituted with one or more R²⁶, CONR⁸R⁹, 3- to 7-membered heterocycloalkyl optionally substituted with one or more R²⁶, C₆-C₁₀ aryl optionally substituted with one or more R²⁶, 5- to 10-membered heteroaryl optionally substituted with one or more R²⁶, OCOC₁-C₆ alkyl optionally substituted with one or more R²⁶, OCOC₆-C₁₀ aryl optionally substituted with one or more R²⁶, OCO(5- to 10-membered heteroaryl) optionally substituted with one or more R²⁶, OCO(3- to 7-membered heterocycloalkyl) optionally substituted with one or more R²⁶, NHCOC₁-C₆ alkyl optionally substituted with one or more R²⁶, NHCOC₆-C₁₀ aryl optionally substituted with one or more R²⁶, NHCO(5- to 10-membered heteroaryl) optionally substituted with one or more R²⁶, NHCO(3- to 7-membered heterocycloalkyl) optionally substituted with one or more R²⁶, NHCOC₂-C₆ alkynyl optionally substituted with one or more R²⁶, C₆-C₁₀ aryloxy optionally substituted with one or more R²⁶, and S(O₂)C₁-C₆ alkyl optionally substituted with one or more R²⁶;
R²⁶ at each occurrence is independently selected from the group consisting of: hydroxy, halo, C₆-C₁₀ aryl, NR⁸R⁹, C₁-C₆ alkyl, and OC₁-C₆ alkyl;
R²⁷, at each occurrence, is independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₁-C₆ haloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms and/or heteroatomic groups in addition to the nitrogen they are attached to;
R¹³ is C₁-C₆ alkyl, C₁-C₆ haloalkyl, C₆-C₁₀ aryl, or 5- to 10-membered heteroaryl; and
each of R¹¹ and R¹² at each occurrence is independently selected from hydrogen and C₁-C₆ alkyl;
or a pharmaceutically acceptable salt thereof.

101. The compound of claim 100, wherein when the compound of Formula AA is a compound of Formula AA-1, X⁴ is other than —X⁵—X⁶— and any two adjacent R¹, R², R³, and R⁴ are taken together with the atoms connecting them to form a ring; and wherein when the compound of Formula AA is a compound of Formula AA-2, any two adjacent R¹, R², and R³ are taken together with the atoms connecting them to form a ring; wherein the ring is selected from the group consisting of:
(i) a monocyclic or bicyclic C₄-C₁₂ cycloalkyl ring optionally substituted with one or more R²⁰, and
(ii) a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹, X², X³, and X⁴, and wherein the heterocycloalkyl ring is optionally substituted with one or more R²⁰.

102. The compound of any one of claims 100-101, wherein when one of X¹ and X⁴ is N;
the other of X¹ and X⁴ is other than O.

103. The compound of any one of claims 100-102, wherein the compound of Formula AA is a compound of Formula AA-1:

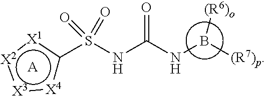

104. The compound of claim any one of claims 100-103, when one of X¹ and X⁴ is N; the other of X¹ and X⁴ is other than O.

105. The compound of any one of claims 100-104, wherein X¹ is CR¹.

106. The compound of any one of claims 100-105, wherein X² is NR².

107. The compound of any one of claims 100-106, wherein X³ is N.

108. The compound of any one of claims 100-107, wherein X⁴ is CR⁴.

109. The compound of any one of claims 100-104, wherein X¹ is CR¹; X² is NR²; X³ is N; and X⁴ is CR⁴.

110. The compound of any one of claims 106-109, wherein R¹ and R², taken together with the atoms connecting them, form a monocyclic or bicyclic C₄-C₁₂ cycloalkyl ring optionally substituted with one or more R²⁰.

111. The compound of any one of claims 106-110, wherein R¹ and R², taken together with the atoms connecting them, form a monocyclic C₅-C₆ cycloalkyl ring optionally substituted with one or more R²⁰.

112. The compound of any one of claims 106-109, wherein R¹ and R², taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X¹ and X², and wherein the heterocycloalkyl ring is optionally substituted with one or more R²⁰.

113. The compound of any one of claims 106-109 and 112, wherein R¹ and R², taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X¹ and X², and wherein the heterocycloalkyl ring is optionally substituted with one or more R²⁰.

114. The compound of claim 113, wherein the monocyclic 5- to-6-membered heterocycloalkyl ring contains 1 O atom.

115. The compound of any one of claims 108-114, wherein R⁴ is H or CH₃.

116. The compound of any one of claims 100-104, wherein X¹ is N.

117. The compound of any one of claims 100-104 and 116, wherein X² is NR².

118. The compound of any one of claims 100-104 and 116-117, wherein X³ is CR³.

119. The compound of any one of claims 100-104 and 116-118, wherein X⁴ is CR⁴.

120. The compound of any one of claims 100-104, wherein X¹ is N; X² is NR²; X³ is CR³; and X⁴ is CR⁴.

121. The compound of any one of claims 118-120, wherein R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

122. The compound of any one of claims 118-121, wherein R² and R³, taken together with the atoms connecting them, form a monocyclic or bicyclic 5- to-12-membered heterocycloalkyl ring containing 1-3 heteroatoms and/or heteroatomic groups independently selected from O, N, NH, and NR¹³ and S, wherein the heteroatom or heteroatomic group is cumulative with the values selected for X² and X³, and wherein the heterocycloalkyl ring is optionally substituted with from 1-2 R²⁰.

123. The compound of any one of claims 118-122, wherein R² and R³, taken together with the atoms connecting them, form a monocyclic or bicyclic 5- to-6-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

124. The compound of any one of claims 118-123, wherein R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X² and X³, and wherein the heterocycloalkyl ring is optionally substituted with one or more R²⁰.

125. The compound of claim 124, wherein the monocyclic 5- to-6-membered heterocycloalkyl ring contains 1 O atom.

126. The compound of any one of claims 119-125, wherein R⁴ is H or CH₃.

127. The compound any one of claims 100-104, wherein X¹ is S.

128. The compound any one of claims 100-104 and 127, wherein X² is CR².

129. The compound any one of claims 100-104 and 127-128, wherein X³ is CR³.

130. The compound any one of claims 100-104 and 127-129, wherein X⁴ is N.

131. The compound any one of claims 100-104, wherein X¹ is S; X² is CR²; X³ is CR³; and X⁴ is N.

132. The compound of any one of claims 128-131, wherein R² and R³, taken together with the atoms connecting them, form a monocyclic or bicyclic C₄-C₁₂ cycloalkyl ring optionally substituted with one or more R²⁰.

133. The compound of any one of claims 128-132, wherein R² and R³, taken together with the atoms connecting them, form a monocyclic C₅-C₆ cycloalkyl ring optionally substituted with one or more R²⁰.

134. The compound of any one of claims 128-131, wherein R² and R³, taken together with the atoms connecting them, form a monocyclic or bicyclic 5- to-12-membered heterocycloalkyl ring containing 1-3 heteroatoms and/or heteroatomic groups independently selected from O, N, NH, NR¹³, and S, wherein the heteroatom or heteroatomic group is cumulative with the values selected for X² and X³, and wherein the heterocycloalkyl ring is optionally substituted with one or more R²⁰.

135. The compound of any one of claims 128-131 and 134, wherein R² and R³, taken together with the atoms connecting them, form a monocyclic 5- to-6-membered heterocycloalkyl ring containing 1 heteroatom or heteroatomic group selected from O, NH, and NR¹³ (e.g., O, NH, or NCH₃), wherein the heteroatom or heteroatomic group is cumulative with the values selected for X² and X³, and wherein the heterocycloalkyl ring is optionally substituted with one or more R²⁰.

136. The compound of claim 135, wherein the monocyclic 5- to-6-membered heterocycloalkyl ring contains 1 heteroatom or heteroatomic group selected from O and NH, wherein the heteroatom or heteroatomic group is cumulative with the values selected for X² and X³.

137. The compound of any one of claims 100-136, wherein R²⁰ is selected from the group consisting of: hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom, wherein the ring is optionally substituted with OS(O)₂Ph.

138. The compound of claim 137, wherein R²¹ is selected from the group consisting of halo (e.g., fluoro), NR⁸R⁹, C₂-C₆ alkynyl (e.g., ethynyl), and C₁-C₆ alkoxy (e.g., methoxy).

139. The compound of any one of claims 100-138, wherein, 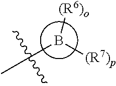
selected from the group consisting of:

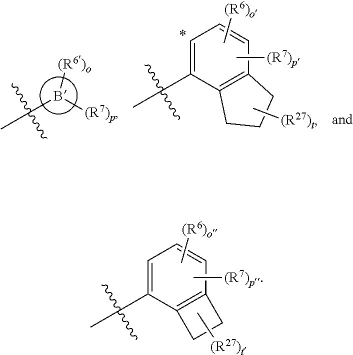

140. The compound of any one of claims 100-139, wherein

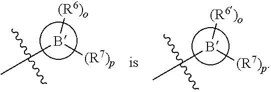

141. The compound of claim 140, wherein o is 2 and p is 2.

142. The compound of any one of claims 140-141, wherein B′ is pyridyl.

143. The compound of any one of claims 140-142, wherein B′ is 2-pyridyl.

144. The compound of any one of claims 140-143, wherein the optionally substituted ring B′ is

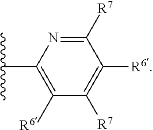

145. The compound of claim 144, wherein two pairs of R^{6′} and R⁷ on adjacent atoms, taken together with the atoms connecting them, independently form a C₄-C₈ cycloalkyl ring or a 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷.

146. The compound of claim 144, wherein the optionally substituted ring B′ is

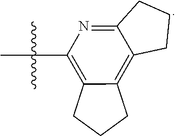 [SMILES: *c1nc2c(c3c1CCC3)CCC2]

147. The compound of any one of claims 140-142, wherein B′ is 4-pyridyl.

148. The compound of any one of claims 140-142 and 147, wherein the optionally substituted ring B′ is

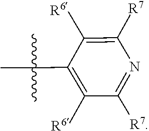

149. The compound of claim 148, wherein at least one pair of R^{6′} and R⁷ on adjacent atoms, taken together with the atoms connecting them, independently form at least one C₄-C₈ cycloalkyl ring or at least one 5- to 8-membered heterocyclic ring containing 1 or 2 heteroatoms and/or heteroatomic groups independently selected from O, N, NH, NR¹³, and S, wherein the cycloalkyl ring or heterocyclic ring is optionally independently substituted with one or more R²⁷.

150. The compound of any one of claims 147-149, wherein, 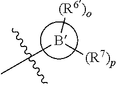
is selected from the group consisting of:

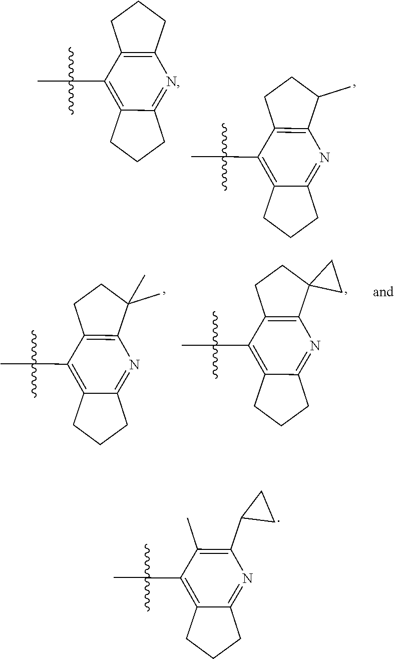

151. The compound of any one of claims 100-139, wherein

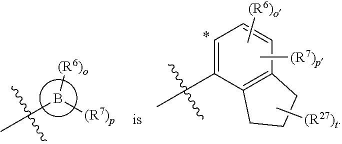

152. The compound of any one of claims 100-139 and 151, wherein

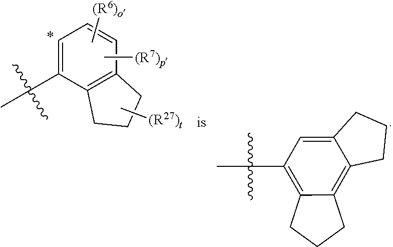

153. The compound of any one of claims 100-139, wherein

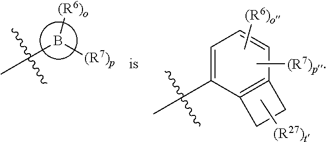

154. The compound of claim 153, wherein

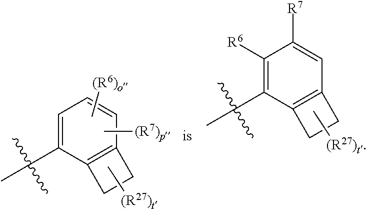

155. The compound of any one of claims 153-154, wherein R⁶ and R⁷, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, N, NH, NR¹³, and S, wherein the carbocyclic ring or heterocyclic ring is optionally independently substituted with one or more R²⁷.

156. The compound of any one of claims 154-155, wherein 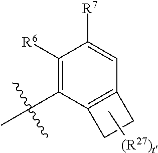
is selected from

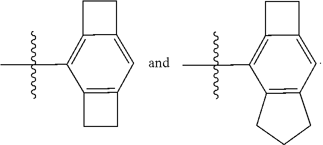 [SMILES: *c1c2c(cc3c1CC3)CC2 ; *c1c2c(cc3c1CC3)CCC2]

157. The compound of any one of claims 100-139, wherein,

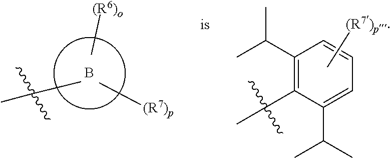

158. The compound of any one of claims 100-139, wherein,

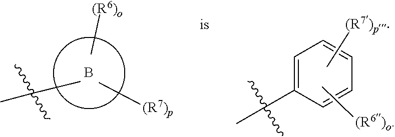

159. The compound of any one of claims 100-104 and 139-158, wherein A is 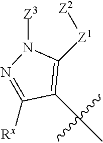
wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or at least one pair of R²⁰ on the same atom, taken together with the atom connecting them, independently forms a monocyclic C₃-C₄ cycloalkyl ring or a monocyclic 3- to 4-membered heterocycloalkyl ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).

160. The compound of any one of claims 100-104 and 139-159, wherein A is selected from the group consisting of:

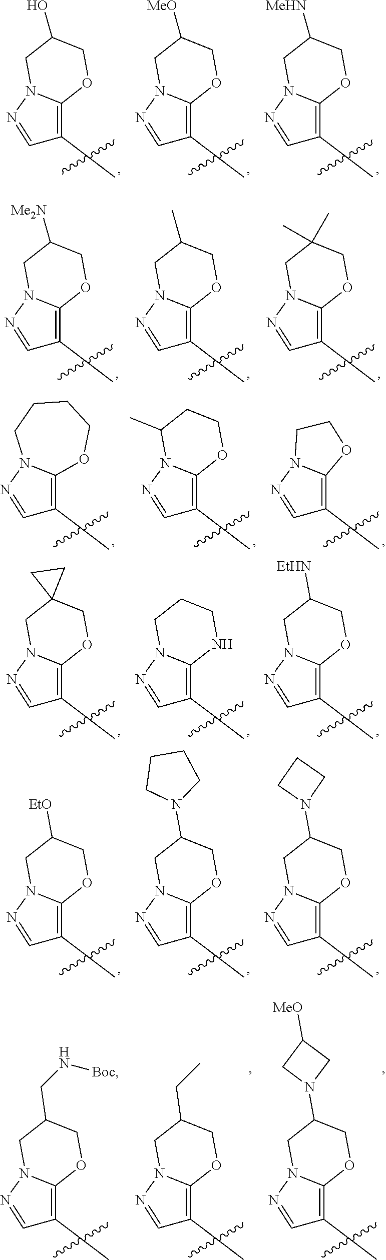

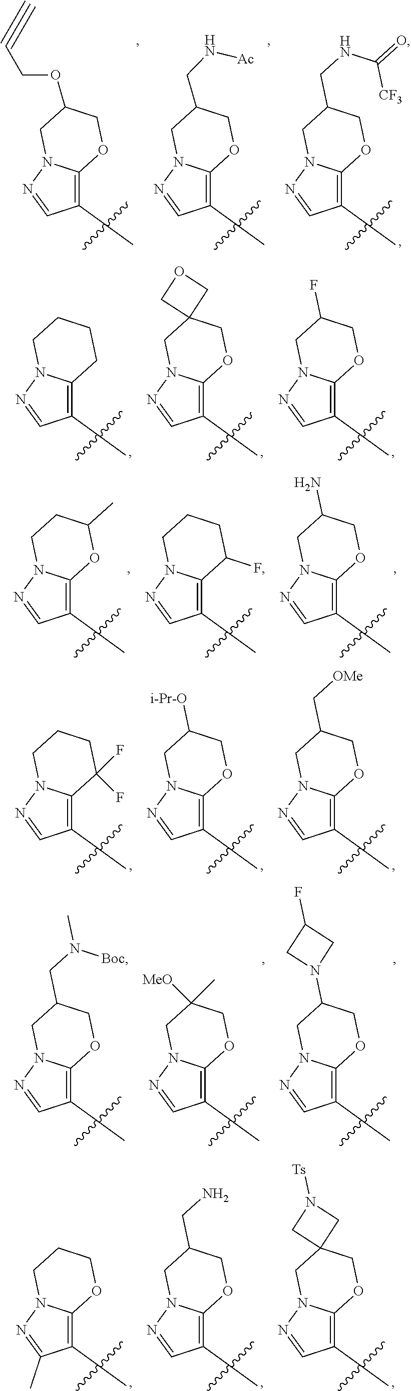

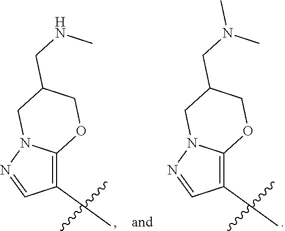 [SMILES: *c1cnn2c1OCC(CNC)C2 ; *c1cnn2c1OCC(CN(C)C)C2]

161. The compound of any one of claims 100-104 and 139-158, wherein A is 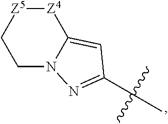
wherein Z⁴ is selected from the group consisting of —CH₂—, —C(O)—, and NH; Z⁵ is selected from the group consisting of O, NH, N—CH₃, and —CH₂—.

162. The compound of any one of claims 100-104, 139-158, and 161, wherein A is selected from the group consisting of:

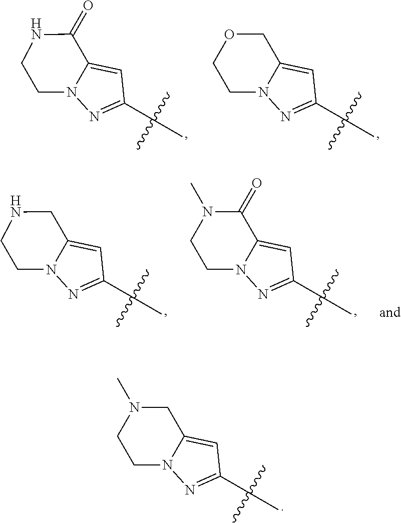 [SMILES: *c1cc2n(n1)CCNC2=O ; *c1cc2n(n1)CCOC2 ; *c1cc2n(n1)CCNC2 ; *c1cc2n(n1)CCN(C)C2=O ; *c1cc2n(n1)CCN(C)C2]

163. The compound of any one of claims 100-102, wherein the compound of Formula AA is a compound of Formula AA-2:

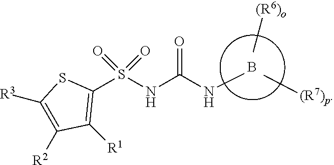

164. The compound of claim 163, wherein R¹ and R² are taken together with the atoms connecting them to form a ring, wherein the ring is selected from the group consisting of:
(i) a monocyclic or bicyclic C₄-C₁₂ cycloalkyl ring optionally substituted with one or more R²⁰,
(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,
(iii) a monocyclic or bicyclic C₆-C₁₀ aryl ring optionally substituted with one or more R²⁰, and
(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

165. The compound of claim 164, wherein the ring is a monocyclic 5-6 membered cycloalkyl ring optionally substituted with one or more R²⁰, or a monocyclic 5-6-membered heterocycloalkyl ring containing 1-3 heteroatoms and/or heteroatomic groups independently selected from O, or NH, wherein the heterocycloalkyl ring is optionally substituted with one or more R²⁰.

166. The compound of any one of claims 164-165, wherein the ring is a monocyclic 5-6-membered heterocycloalkyl ring containing one heteroatom or heteroatomic group independently selected from O, or NH, wherein the heterocycloalkyl ring is optionally substituted with one or more (e.g., two) R²⁰.

167. The compound of any one of claims 164-165, wherein R²⁰ is C₁-C₆ alkyl (e.g., methyl).

168. The compound of any one of claims 164-167, wherein A is selected from the group consisting of:

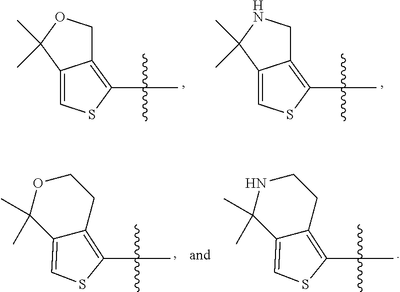 [SMILES: *c1scc2c1COC2(C)C ; *c1scc2c1CNC2(C)C ; *c1scc2c1CCOC2(C)C ; *c1scc2c1CCNC2(C)C]

169. The compound of claim 163, wherein R² and R³ are taken together with the atoms connecting them to form a ring, wherein the ring is selected from the group consisting of:
(i) a monocyclic or bicyclic C₄-C₁₂ cycloalkyl ring optionally substituted with one or more R²⁰,
(ii) a monocyclic or bicyclic 5- to-12-membered heterocycloalkyl ring containing 1-3 heteroatoms and/or heteroatomic groups independently selected from O, N, NH, NR¹³, and S, wherein the heterocycloalkyl ring is optionally substituted with one or more R²⁰,
(iii) a monocyclic or bicyclic C₆-C₁₀ aryl ring optionally substituted with one or more R²⁰, and
(iv) a monocyclic or bicyclic 5-10 membered heteroaryl ring containing 1-3 heteroatoms and/or heteroatomic groups independently selected from O, N, NH, NR¹³, and S, wherein the heteroaryl ring is optionally substituted with one or more R²⁰.

170. The compound of claim 169, wherein the ring is a monocyclic 5-6 membered cycloalkyl ring optionally substituted with one or more R²⁰, or a monocyclic 5-6-membered heterocycloalkyl ring containing 1-3 heteroatoms and/or heteroatomic groups independently selected from O, or NH, wherein the heterocycloalkyl ring is optionally substituted with one or more R²⁰.

171. The compound of any one of claims 169-170, wherein the ring is a monocyclic 5-6-membered heterocycloalkyl ring containing one heteroatom or heteroatomic group independently selected from O or NH, wherein the heterocycloalkyl ring is optionally substituted with one or more (e.g., two) R²⁰.

172. The compound of any one of claims 169-171, wherein R²⁰ is selected from hydroxy, C₁-C₆ alkyl (e.g., methyl), and NR⁸R⁹.

173. The compound of any one of claims 169-172, wherein A is selected from the group consisting of:

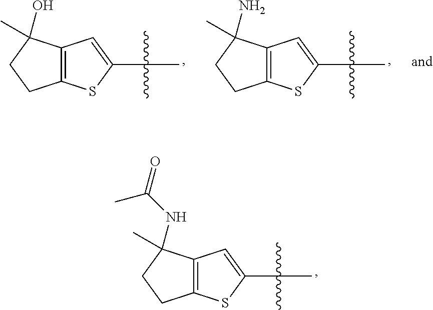 [SMILES: *c1cc2c(s1)CCC2(C)O ; *c1cc2c(s1)CCC2(C)N ; *c1cc2c(s1)CCC2(C)NC(C)=O]

174. The compound of any one of claims 163-173, where o is 2 and p is 2.

175. The compound of any one of claims 163-174, wherein the optionally substituted ring B is

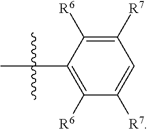

176. The compound of claim 175, wherein two pairs of R⁶ and R⁷ on adjacent atoms, taken together with the atoms connecting them, independently form two C₄-C₈ carbocyclic rings or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

177. The compound of any one of claims 175-176, wherein two pairs of R⁶ and R⁷ on adjacent atoms, taken together with the atoms connecting them, independently form two C₅ carbocyclic rings optionally independently substituted with one or more R²⁷.

178. The compound of any one of claims 174-176, wherein the optionally substituted ring B is

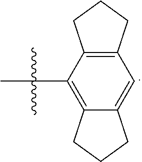 [SMILES: *c1c2c(cc3c1CCC3)CCC2]

179. The compound of any one of claims 163-173, wherein the optionally substituted ring B is

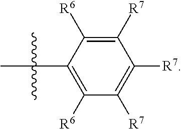

180. The compound of claim 179, wherein two pairs of R⁶ and R⁷ on adjacent atoms, taken together with the atoms connecting them, independently form two C₄-C₈ carbocyclic ring or two 5- to 8-membered heterocyclic ring containing 1 or 2 heteroatoms and/or heteroatomic groups independently selected from O, N, NH, NR¹³, and S, wherein the carbocyclic rings or heterocyclic rings are optionally independently substituted with one or more R²⁷.

181. The compound of any one of claims 179-180, wherein two pairs of R⁶ and R⁷ on adjacent atoms, taken together with the atoms connecting them, independently form two C₅ carbocyclic rings optionally independently substituted with one or more R²⁷.

182. The compound of any one of claims 179-179, wherein the optionally substituted ring B is

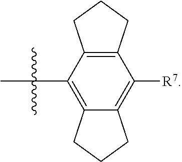

183. A compound selected from the compounds in Table 1A, Table 1B, Table 1C, and pharmaceutically acceptable salts thereof.

184. A pharmaceutical composition comprising a compound or salt as claimed in any one of claims 1-183 and one or more pharmaceutically acceptable excipients.

185. A method for modulating NLRP3 activity, the method comprising contacting NLRP3 with an effective amount of a compound as claimed in any one of claims 1-183 or a pharmaceutical composition as claimed in claim 184.

186. The method of claim 185, wherein the modulating comprises antagonizing NLRP3.

187. The method of any one of claims 185 and 186, which is carried out in vitro.

188. The method of any one of claims 185-187, wherein the method comprises contacting a sample comprising one or more cells comprising NLRP3 with the compound.

189. The method of any one of claims 185-186 and 188, which is carried out in vivo.

190. The method of claim 189, wherein the method comprises administering the compound to a subject having a disease in which NLRP3 signaling contributes to the pathology and/or symptoms and/or progression of the disease.

191. The method of claim 190, wherein the subject is a human.

192. A method of treating a disease, disorder or condition that is a metabolic disorder, comprising administering to a subject in need of such treatment an effective amount of a compound as claimed in any one of claims 1-183 or a pharmaceutical composition as claimed in claim 184.

193. The method of claim 192, wherein the metabolic disorder is Type 2 diabetes, atherosclerosis, obesity or gout.

194. A method of treating a disease, disorder or condition that is a disease of the central nervous system, comprising administering to a subject in need of such treatment an effective amount of a compound as claimed in any one of claims 1-183 or a pharmaceutical composition as claimed in claim 184.

195. The method of claim 194, wherein the disease of the central nervous system is Alzheimer's disease, multiple sclerosis, Amyotrophic Lateral Sclerosis or Parkinson's disease.

196. A method of treating a disease, disorder or condition that is lung disease, comprising administering to a subject in need of such treatment an effective amount of a compound as claimed in any one of claims 1-183 or a pharmaceutical composition as claimed in claim 184.

197. The method of claim 196, wherein the lung disease is asthma, COPD or pulmonary idiopathic fibrosis.

198. A method of treating a disease, disorder or condition that is liver disease, comprising administering to a subject in need of such treatment an effective amount of a compound as claimed in any one of claims 1-183 or a pharmaceutical composition as claimed in claim 184.

199. The method of claim 198, wherein the liver disease is NASH syndrome, viral hepatitis or cirrhosis.

200. A method of treating a disease, disorder or condition that is pancreatic disease, comprising administering to a subject in need of such treatment an effective amount of a compound as claimed in any one of claims 1-183 or a pharmaceutical composition as claimed in claim 184.

201. The method of claim 200, wherein the pancreatic disease is acute pancreatitis or chronic pancreatitis.

202. A method of treating a disease, disorder or condition that is kidney disease, comprising administering to a subject in need of such treatment an effective amount of a compound as claimed in any one of claims 1-183 or a pharmaceutical composition as claimed in claim 184.

203. The method of claim 202, wherein the kidney disease is acute kidney injury or chronic kidney injury.

204. A method of treating a disease, disorder or condition that is intestinal disease, comprising administering to a subject in need of such treatment an effective amount of a compound as claimed in any one of claims 1-183 or a pharmaceutical composition as claimed in claim 184.

205. The method of claim 204, wherein the intestinal disease is Crohn's disease or Ulcerative Colitis.

206. A method of treating a disease, disorder or condition that is skin disease, comprising administering to a subject in need of such treatment an effective amount of a compound as claimed in any one of claims 1-183 or a pharmaceutical composition as claimed in claim 184.

207. The method of claim 206, wherein the skin disease is psoriasis.

208. A method of treating a disease, disorder or condition that is musculoskeletal disease, comprising administering to a subject in need of such treatment an effective amount of a compound as claimed in any one of claims 1-183 or a pharmaceutical composition as claimed in claim 184.

209. The method of claim 208, wherein the musculoskeletal disease is scleroderma.

210. A method of treating a disease, disorder or condition that is a vessel disorder, comprising administering to a subject in need of such treatment an effective amount of a compound as claimed in any one of claims 1-183 or a pharmaceutical composition as claimed in claim 184.

211. The method of claim 210, wherein the vessel disorder is giant cell arteritis.

212. A method of treating a disease, disorder or condition that is a disorder of the bones, comprising administering to a subject in need of such treatment an effective amount of a compound as claimed in any one of claims 1-183 or a pharmaceutical composition as claimed in claim 184.

213. The method of claim 212, wherein the disorder of the bones is osteoarthritis, osteoporosis or osteopetrosis disorders.

214. A method of treating a disease, disorder or condition that is eye disease, comprising administering to a subject in need of such treatment an effective amount of a compound as claimed in any one of claims 1-183 or a pharmaceutical composition as claimed in claim 184.

215. The method of claim 214, wherein the eye disease is glaucoma or macular degeneration.

216. A method of treating a disease, disorder or condition that is a disease caused by viral infection, comprising administering to a subject in need of such treatment an effective amount of a compound as claimed in any one of claims 1-183 or a pharmaceutical composition as claimed in claim 184.

217. The method of claim 216, wherein the diseases caused by viral infection is HIV or AIDS.

218. A method of treating a disease, disorder or condition that is an autoimmune disease, comprising administering to a subject in need of such treatment an effective amount of a compound as claimed in any one of claims 1-183 or a pharmaceutical composition as claimed in claim 184.

219. The method of claim 218, wherein the autoimmune disease is Rheumatoid Arthritis, Systemic Lupus Erythematosus, Autoimmune Thyroiditis, Addison's disease, or pernicious anemia.

220. A method of treating a disease, disorder or condition that is cancer or aging, comprising administering to a subject in need of such treatment an effective amount of a compound as claimed in any one of claims 1-183 or a pharmaceutical composition as claimed in claim 184.

221. A method of treating a disease, disorder or condition that is a cancer selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML); chronic myeloid leukemia (CML); gastric cancer; and lung cancer metastasis, comprising administering to a subject in need of such treatment an effective amount of a compound as claimed in any one of claims 1-183 or a pharmaceutical composition as claimed in claim 184.

222. The method of claim 221, wherein the cancer is MDS.

223. The method of claim 221, wherein the cancer is non-small lung cancer.

224. The method of claim 221, wherein the cancer is acute lymphoblastic leukemia.

225. The method of claim 221, wherein the cancer is LCH.

226. The method of claim 221, wherein the cancer is multiple myeloma.

227. The method of claim 221, wherein the cancer is promyelocytic leukemia.

228. The method of claim 221, wherein the cancer is acute myeloid leukemia (AML).

229. The method of claim 221, wherein the cancer is chronic myeloid leukemia (CML).

230. The method of claim 221, wherein the cancer is gastric cancer.

231. The method of claim 221, wherein the cancer is lung cancer metastasis.

232. The method of any one of claims 190-231, further comprising administering a therapeutically effective amount of an anti-TNFα agent to the subject.

233. The method of claim 232, wherein the NLRP3 antagonist is administered to the subject prior to administration of the anti-TNFα agent to the subject.

234. The method of claim 232, wherein the anti-TNFα agent is administered to the subject prior to the administration of the NLRP3 antagonist to the subject.

235. The method of claim 232, wherein the NLRP3 antagonist and the anti-TNFα agent are administered to the subject at substantially the same time.

236. The method of claim 232, wherein the NLRP3 antagonist and the anti-TNFα agent are formulated together in a single dosage form.

237. The compound of claim 1, wherein
A is 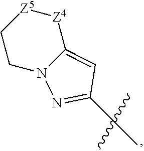
wherein Z⁴ is selected from the group consisting of —CH₂—, —C(O)—, and NH; Z⁵ is selected from the group consisting of O, NH, N—CH₃, and —CH₂—; and
the B is

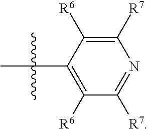

238. The compound of claim 237, wherein the B is selected from the group consisting of: 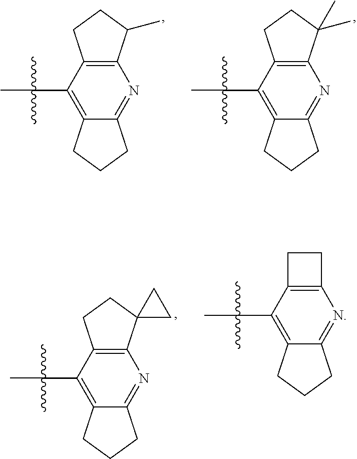 [SMILES: *c1c2c(nc3c1CCC3C)CCC2 ; *c1c2c(nc3c1CCC3(C)C)CCC2 ; *c1c2c(nc3c1CCC31CC1)CCC2 ; *c1c2c(nc3c1CC3)CCC2]
